SEQUENCE LISTING (6 sequences)

SEQ ID NO: 1 - protein, 306 aa, SARS-CoV-2 [sequence omitted]

SEQ ID NO: 2 - protein, 306 aa, SARS-CoV
SGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQ

SEQ ID NO: 3 - protein, 15 aa, synthetic construct
  REGION 1..15: note = Synthetic Construct
KTSAVLQSGFRKMEK

SEQ ID NO: 4 - protein, 306 aa, MERS-CoV
SGLVKMSHPSGDVEACMVQVTCGSMTLNGLWLDNTVWCPRHVMCPADQLSDPNYDALLISMTNHSFSVQKHIGAPANLRVVGHAMQGTLLKLTVDVANPSTPAYTFTTVKPGAAFSVLACYNGRPTGTFTVVMRPNYTIKGSFLCGSCGSVGYTKEGSVINFCYMHQMELANGTHTGSAFDGTMYGAFMDKQVHQVQLTDKYCSVNVVAWLYAAILNGCAWFVKPNRTSVVSFNEWALANQFTEFVGTQSVDMLAVKTGVAIEQLLYAIQQLYTGFQGKQILGSTMLEDEFTPEDVNMQIMGVVMQ

SEQ ID NO: 5 - protein, 302 aa, HCoV-229E
AGLRKMAQPSGFVEKCVVRVCYGNTVLNGLWLGDIVYCPRHVIASNTTSAIDYDHEYSIMRLHNFSIISGTAFLGVVGATMHGVTLKIKVSQTNMHTPRHSFRTLKSGEGFNILACYDGCAQGVFGVNMRTNWTIRGSFINGACGSPGYNLKNGEVEFVYMHQIELGSGSHVGSSFDGVMYGGFEDQPNLQVESANQMLTVNVVAFLYAAILNGCTWWLKGEKLFVEHYNEWAQANGFTAMNGEDAFSILAAKTGVCVERLLHAIQVLNNGFGGKQILGYSSLNDEFSINEVVKQMFGVNLQ

SEQ ID NO: 6 - protein, 302 aa, HCoV-OC43
SGIVKMVNPTSKVEPCVVSVTYHNMTLNGLWLDDKVYCPRHVICSASDMTNPDYTNLLCVTSSDFTVLFDRLSLTVMSYQMRGCMLVLTVTLQNSRTPKYTFGVVKPGETFTVLAAYNGKPQGAFHVTMRSSYTIKGSFLCGSCGSVGYVIMGDCVKFVYMHQLELSTGCHTGTDFNGDFYGPYKDAQVVQLPIQDYIQSVNFLAWLYAAILNNCNWFIQSDKCSVEDFNVMALSNGFSQVKSDLVIDALASMTGVSLETLLAAIKRLKNGFQGRQIMGSCSFEDELTPSDVYQQLAGIKLQ

(19) United States
(12) Patent Application Publication
(10) Pub. No.: US 2025/0059171 A1
(43) Pub. Date: Feb. 20, 2025

(54) ANTIVIRAL COMPOUNDS

(71) Applicant: Hoffmann-La Roche Inc., Little Falls, NJ (US)

(72) Inventors: David EDMONDS, Riehen (CH); Chungen LIANG, Shanghai (CN); Hongying YUN, Shanghai (CN); Bo ZHANG, Shanghai (CN); Xiufang ZHENG, Shanghai (CN)

(73) Assignee: Hoffmann-La Roche Inc., Little Falls, NJ (US)

(21) Appl. No.: 18/736,151

(22) Filed: Jun. 6, 2024

Related U.S. Application Data
(63) Continuation of application No. PCT/EP2022/084782, filed on Dec. 7, 2022.

(30) Foreign Application Priority Data
Dec. 9, 2021 (WO) PCT/CN2021/136794
Mar. 2, 2022 (WO) PCT/CN2022/078808
Nov. 2, 2022 (EP) 22205032.0

(51) Int. Cl. C07D 403/14; A61K 31/4015; A61K 31/403; A61K 31/4155; A61K 31/422; A61K 31/4439; A61K 31/454; A61K 31/506; A61P 31/14; C07D 207/267; C07D 209/52; C07D 401/12; C07D 401/14; C07D 403/12; C07D 413/14

(52) U.S. Cl. CPC C07D 403/14; A61K 31/4015; A61K 31/403; A61K 31/4155; A61K 31/422; A61K 31/4439; A61K 31/454; A61K 31/506; A61P 31/14; C07D 207/267; C07D 209/52; C07D 401/12; C07D 401/14; C07D 403/12; C07D 413/14

(57) ABSTRACT

The invention provides viral protease inhibitors having the general formula (I)

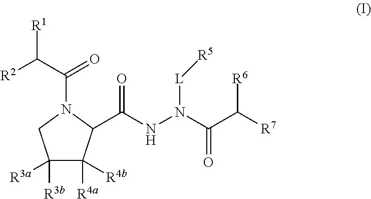

wherein the variables are as described herein, compositions including the compounds, processes of manufacturing the compounds and methods of using the compounds.

### CROSS REFERENCE TO RELATED APPLICATIONS

This application is a continuation of International Application No. PCT/EP2022/084782, filed Dec. 7, 2022, which claims priority to Chinese Patent Application No. PCT/CN2021/136794 filed Dec. 9, 2021 and Chinese Patent Application No. PCT/CN2022/078808 filed Mar. 2, 2022 and European Patent Application No. 22205032.0 filed Nov. 2, 2022, all of which are incorporated herein by reference in their entirety.

### SEQUENCE LISTING

This application contains a Sequence Listing which has been submitted electronically in XML format and is hereby incorporated by reference in its entirety. Said XML copy, created on May 21, 2024, is named P37161-US Sequence Listing.xml and is 7,832 bytes in size.

### FIELD OF THE INVENTION

The present invention relates to peptidomimetic (or peptide-like) compounds, specifically viral protease inhibitors, for the treatment of viral infections, and methods of preparing and using such compounds.

### BACKGROUND OF THE INVENTION

In December 2019, a new coronavirus, named severe acute respiratory syndrome coronavirus 2 (SARS-CoV-2), caused an outbreak of the novel coronavirus disease COVID-19, which has spread to more than 200 countries with over 9 million confirmed cases and over 479,133 confirmed deaths worldwide as of Jun. 26, 2020 (WHO COVID-19 situation report −157). The WHO declared the coronavirus outbreak a public health emergency of international concern. Currently, there are no clinically effective vaccine or specific antiviral drug available for the prevention and treatment of SARS-CoV-2 infections.

Coronaviruses (CoVs) are enveloped, positive-sense, single-stranded RNA viruses. Seven human coronaviruses (HCoVs) have been so far identified, namely HCoV-229E, HCoV—OC43, HCoV-NL63, HCoV—HKU1, SARS coronavirus (SARS-CoV), Middle East respiratory syndrome coronavirus (MERS-CoV) and the novel coronavirus (SARS-CoV-2). While SARS-CoV, MERS-CoV, and SARS-CoV-2 are highly pathogenic, the others generally cause mild to moderate upper-respiratory tract illness and contribute to 15%-30% cases of common colds in human adults.

The RNA genome of SARS-CoV-2 is about 30 kilobases in length shares approximately 80% sequence identity with SARS-CoV (Zhou P. et al. “A pneumonia outbreak associated with a new coronavirus of probable bat origin.” Nature 579(7798): 270-273, 2020). It consists six major open-reading frames (ORFs). ORF 1a/b, which is about two thirds of the whole genome length, directly translates two polyproteins, pp1a and pp1ab, which encodes 16 nonstructural proteins (nsps) to form the replication transcription complex. Nsp3, which encodes papain-like protease (PL^{pro}), and nsp5, which encodes 3-chymotrypsin-like cysteine protease (3CL^{pro}, also known as main protease, M^{pro}), are essential for processing these polyproteins. 3CL^{pro} cleaves the polyprotein at 11 distinct sites to generate various nsps that are important for viral replication. Accordingly, inhibitors that block the cleavage function of 3CL^{pro} could inhibit virus replication. In addition, 3CL^{pro} is highly conserved between SARS-CoV and SARS-CoV-2 (96% sequence identity), as well as the other human coronaviruses. Furthermore, no human proteases with a similar cleavage specificity is known. These desired properties make 3CL^{pro} one of the most attractive targets against coronavirus infections.

### SUMMARY OF THE INVENTION

In a first aspect, the present invention provides compounds of formula (I)

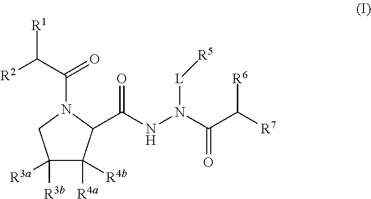

wherein the variables are as defined herein.

In one aspect, the present invention provides a process of manufacturing a compound of formula (I) described herein, or a pharmaceutically acceptable salt thereof, wherein said process is as described in Schemes 1 to 3 below.

In a further aspect, the present invention provides a compound of formula (I) as described herein, or a pharmaceutically acceptable salt thereof, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use in the treatment or prophylaxis of coronavirus infections.

In a further aspect, the present invention provides a compound of formula (I) described herein, or a pharmaceutically acceptable salt thereof, for use in inhibiting the enzymatic activity of 3C-like proteases.

### BRIEF DESCRIPTION OF THE FIGURES

FIG. 1 shows the crystal structure of example 165a as assessed by single crystal X-ray diffraction.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl, tert-butyl, and 2,2-dimethylpropyl.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃₋₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl. A particularly preferred, yet non-limiting example of cycloalkyl is cyclopropyl.

The term “cycloalkylalkyl” refers to a cycloalkyl group that is bound to the parent molecule via an alkylene group. A particularly preferred, yet non-limiting example of cycloalkylalkyl is 1-bicyclo[1.1.1]pentanylmethyl.

The term “alkylcycloalkyl” refers to a cycloalkyl group, wherein at least one of the hydrogen atoms of the cycloalkyl group has been replaced by an alkyl group. Preferably, “alkylcycloalkyl” refers to a cycloalkyl group wherein 1, 2 or 3 hydrogen atoms of the cycloalkyl group have been replaced by an alkyl group. A particularly preferred, yet non-limiting example of alkylcycloalkyl is a cycloalkyl group wherein 1 of the hydrogen atoms of the cycloalkyl group have been replaced by an alkyl group, such as 1-methylcyclopropyl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “arylalkyl” refers to an aryl group that is bound to the parent molecule via an alkylene group. A particularly preferred, yet non-limiting example of arylalkyl is benzyl.

The term “aryloxy” refers to an aryl group that is bound to the parent molecule via an oxygen atom. A non-limiting example of aryloxy is phenoxy.

The term “heteroaryl” refers to a mono- or multivalent, monocyclic, bicyclic or tricyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O, S and N. Some non-limiting examples of heteroaryl include spiro[cyclopropane-1,3′-indoline](e.g., spiro[cyclopropane-1,3′-indoline]-1′-yl), 2-pyridyl, 3-pyridyl, 4-pyridyl, pyrazin-2-yl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, 1,2,4-oxadiazolyl, 1,3,4-oxadiazolyl, thiazol-2-yl, thiazol-4-yl, thiazol-5-yl, pyridazin-3-yl, pyridazin-4-yl, 1,2,4-triazol-4-yl, 1,2,4-triazol-1-yl, 4H-1,2,4-triazol-3-yl, 4,5,6,7-tetrahydroindazol-2-yl, 6,7-dihydro-4H-pyrano[4,3-c]pyrazol-2-yl, thiazolyl, benzofurazan-4-yl, tetrazolyl, isoxazolyl, and morpholinyl. Particularly preferred, yet non-limiting examples of heteroaryl are pyridyl, pyrazinyl, pyrimidinyl, pyrazolyl, imidazolyl, oxazolyl, isoxazolyl, oxadiazolyl and triazolyl.

The term heteroarylalkyl refers to a heteroaryl group that is bound to the parent molecule via an alkylene group. A particularly preferred, yet non-limiting example of heteroarylalkyl is pyridylmethyl.

The term “heterocyclyl” or “heterocycloalkyl” refers to a saturated or partly unsaturated mono- or bicyclic, preferably monocyclic ring system of 3 to 14 ring atoms, preferably 3 to 10 ring atoms, more preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidinyl, pyrrolidinyl, oxetanyl, 5-azaspiro[2.5]octan-5-yl, piperidyl, 3,3a,4,5,6,6a-hexahydro-TH-cyclopenta[c]pyrrol-2-yl, 2-azaspiro[3.3]heptan-2-yl, 2,6-diazaspiro[3.3]heptanyl, 2-azaspiro[3.5]nonan-2-yl, 1,2-dihydropyridiynl, piperidyl, pyrrolidinyl, and thietanyl.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl, difluoromethyl, 1,1-difluoroethyl, 2,2-difluoroethyl, and 2,2,2-trifluoroethyl.

The term “oxo” refers to a double bonded oxygen (═O).

The term “carbamoyl” refers to a group H₂N—C(O)—.

The term “acyl” refers to a group CH₃—C(O)—.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

### Compounds of the Invention

In a first aspect, the present invention provides a compound of Formula (I)

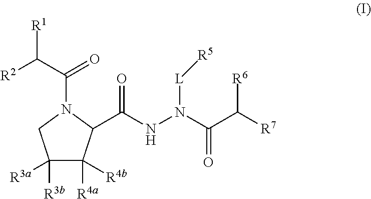

or a pharmaceutically acceptable salt thereof, wherein:
L is C₁-C₆-alkyl;
L¹ is selected from a covalent bond, O, NH, and

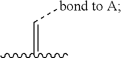

A is selected from C₆-C₁₄-aryl and 3- to 14-membered heteroaryl;
R¹ is selected from a group

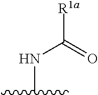

and a group

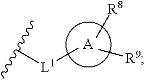

and
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl, wherein said C₃-C₁₀-cycloalkyl is optionally substituted with 1 C₁-C₆-alkyl substituent; or
R¹ and R², taken together with the carbon atom to which they are attached, form a 3- to 14-membered heteroaryl or a C₆-C₁₄-aryl, wherein said 3- to 14-membered heteroaryl or C₆-C₁₄-aryl is optionally substituted with 1 to 2 substituents selected from halogen and halo-C₁-C₆-alkyl;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl and halo-C₁-C₆-alkyl;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C₃-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from 3- to 14-membered heterocycloalkyl, 3- to 14-membered heteroaryl, carbamoyl, and C₁-C₆-alkyl-NH—C(O)—; wherein said 3- to 14-membered heterocycloalkyl is optionally substituted with 1 oxo substituent;
R⁶ is selected from fluoro and chloro;
R⁷ is selected from hydrogen, chloro, and acyl; and
R⁸ and R⁹ are each independently selected from hydrogen, halogen and halo-C₁-C₆-alkyl.

In one embodiment, the present invention provides a compound of formula (I) as described herein, or a pharmaceutically acceptable salt thereof, wherein:
L is C₁-C₆-alkyl;
R¹ is selected from a group

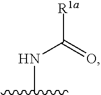

C₆-C₁₄-aryloxy, and a 3- to 14-membered heteroaryl; wherein said C₆-C₁₄-aryloxy and said 3- to 14-membered heteroaryl are optionally substituted with 1 to 2 substituents selected from halogen and halo-C₁-C₆-alkyl; and
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl, wherein said C₃-C₁₀-cycloalkyl is optionally substituted with 1 C₁-C₆-alkyl substituent; or
R¹ and R², taken together with the carbon atom to which they are attached, form a 3- to 14-membered heteroaryl, wherein said 3- to 14-membered heteroaryl is optionally substituted with 1 halo-C₁-C₆-alkyl substituent;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl and halo-C₁-C₆-alkyl;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C₃-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from 3- to 14-membered heterocycloalkyl, 3- to 14-membered heteroaryl, carbamoyl, and C₁-C₆-alkyl-NH—C(O)—; wherein said 3- to 14-membered heterocycloalkyl is optionally substituted with 1 oxo substituent;
R⁶ is selected from fluoro and chloro; and
R⁷ is selected from hydrogen, chloro, and acyl.

In one embodiment, the present invention provides a compound of formula (I) as described herein, or a pharmaceutically acceptable salt thereof, wherein:
L is C₁-C₆-alkyl;
R¹ is selected from a group

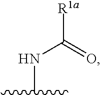

C₆-C₁₄-aryloxy, and a 3- to 14-membered heteroaryl; wherein said C₆-C₁₄-aryloxy and said 3- to 14-membered heteroaryl are optionally substituted with 1 to 2 substituents selected from halogen and halo-C₁-C₆-alkyl; and
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl; or
R¹ and R², taken together with the carbon atom to which they are attached, form a 3- to 14-membered heteroaryl, wherein said 3- to 14-membered heteroaryl is optionally substituted with 1 halo-C₁-C₆-alkyl substituent;
R^{1a} is selected from C₃-C₁₀-cycloalkyl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 halo-C₁-C₆-alkyl substituent;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C₃-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from 3- to 14-membered heterocycloalkyl, 3- to 14-membered heteroaryl, carbamoyl, and C₁-C₆-alkyl-NH—C(O)—; wherein said 3- to 14-membered heterocycloalkyl is optionally substituted with 1 oxo substituent;
R⁶ is selected from fluoro and chloro; and
R⁷ is selected from hydrogen, chloro, and acyl.

In one embodiment, the compound of formula (I) of the present invention is a compound of formula (II)

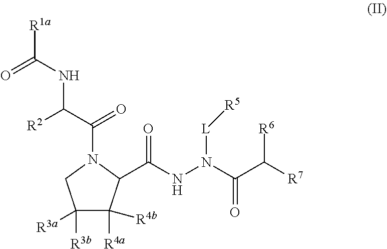

or a pharmaceutically acceptable salt thereof, wherein all variables are as defined herein.

In one embodiment, the compound of formula (I) of the present invention is a compound of formula (III)

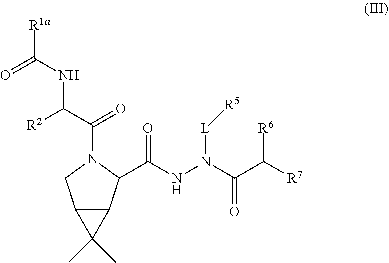

or a pharmaceutically acceptable salt thereof, wherein all variables are as defined herein.

In one embodiment, the compound of formula (I) of the present invention is a compound of formula (IV)

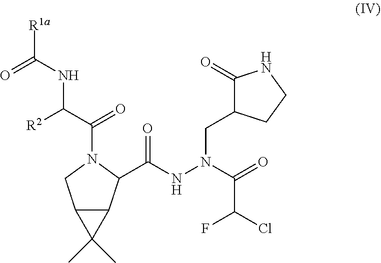

or a pharmaceutically acceptable salt thereof, wherein all variables are as defined herein.

In one embodiment, the compound of formula (I) of the present invention is a compound of formula (V)

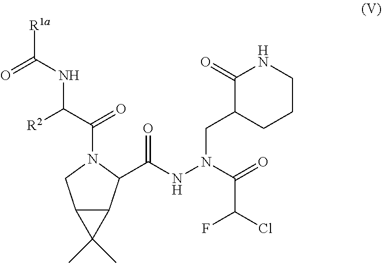

or a pharmaceutically acceptable salt thereof, wherein all variables are as defined herein.

In one embodiment, the compound of formula (I) of the present invention is a compound of formula (VI)

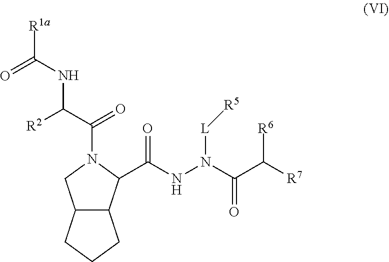

or a pharmaceutically acceptable salt thereof, wherein all variables are as defined herein.

In one embodiment, the compound of formula (I) of the present invention is a compound of formula (VII)

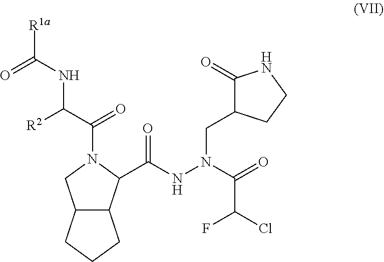

or a pharmaceutically acceptable salt thereof, wherein all variables are as defined herein.

In one embodiment, the compound of formula (I) of the present invention is a compound of formula (VIII)

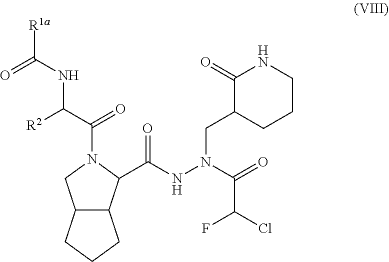

or a pharmaceutically acceptable salt thereof, wherein all variables are as defined herein.

In one embodiment, the present invention provides a compound of formula (I) as described herein, or a pharmaceutically acceptable salt thereof, wherein:
L¹ is selected from a covalent bond, O, NH, and

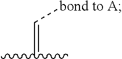

A is selected from C₆-C₁₄-aryl and 3- to 14-membered heteroaryl;
R¹ is selected from a group

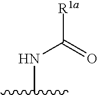

and a group

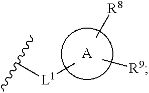

and
R² is selected from hydrogen, C₃-C₁₀-cycloalkyl, C₃-C₁₀-cycloalkyl-C₁-C₆-alkyl, (3- to 14-membered heteroaryl)-C₁-C₆-alkyl, C₁-C₆-alkyl, C₆-C₁₄-aryl, C₆-C₁₄-aryl-C₁-C₆-alkyl, and C₁-C₆-alkyl-S—C₁-C₆-alkyl, wherein said C₃-C₁₀-cycloalkyl is optionally substituted with 1 C₁-C₆-alkyl substituent; or
R¹ and R², taken together with the carbon atom to which they are attached, form a C₆-C₁₄-aryl which is substituted with 2 substituents selected from halogen and halo-C₁-C₆-alkyl;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl and halo-C₁-C₆-alkyl; and
R⁸ and R⁹ are each independently selected from hydrogen, halogen and halo-C₁-C₆-alkyl.

In one embodiment, the present invention provides a compound of formula (I) as described herein, or a pharmaceutically acceptable salt thereof, wherein:
L¹ is selected from a covalent bond, O, NH, and

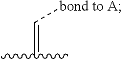

A is selected from phenyl, pyridyl, pyrimidinyl;
R¹ is selected from a group

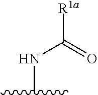

and a group

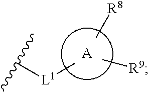

and
R² is selected from hydrogen, cyclopropyl, 1-methylcyclopropyl, cyclobutyl, 1-bicyclo[1.1.1]pentanylmethyl, methyl, ethyl, propyl, sec-butyl, tert-butyl, isobutyl, 1-ethylpropyl, 1,1-dimethylpropyl, 2,2-dimethylpropyl, 2-methylsulfanylethyl, phenyl, benzyl, and pyridylmethyl; or
R¹ and R², taken together with the carbon atom to which they are attached, form a phenyl, which is substituted with 2 substituents selected from fluoro and CF₃;
R^{1a} is selected from bicyclo[1.1.1]pentanyl, cyclopropyl, 1-fluorocyclopropyl, 2,2-difluorocyclopropyl, 1-methylclopropyl, 1-fluorocyclobutyl, 3,3-difluorocyclobutyl, cyclobutyl, phenyl, isoxazolyl, methyl, ethyl, propyl, isopropyl, isobutyl, sec-butyl, tert-butyl, CHFCl, CHCl₂, CHF₂, CF₃, 1-fluoro-1-methyl-ethyl, 2,2-difluoroethyl, 2,2,2-trifluoroethyl, 1-fluoroethyl, and 1,1,2,2,2-pentachloroethyl; and
R⁸ and R⁹ are each independently selected from hydrogen, fluoro and CF₃.

In one embodiment, the present invention provides a compound of formula (I) as described herein, or a pharmaceutically acceptable salt thereof, wherein: bond to A
L¹ is selected from a covalent bond, O, NH, and

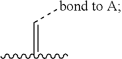

A is selected from phenyl, pyridyl, pyrimidinyl;
R¹ is selected from a group

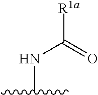

and a group

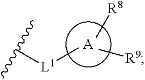

and
R² is selected from hydrogen, cyclopropyl, 1-methylcyclopropyl, cyclobutyl, 1-bicyclo[1.1.1]pentanylmethyl, methyl, ethyl, propyl, sec-butyl, tert-butyl, isobutyl, 1-ethylpropyl, 1,1-dimethylpropyl, 2,2-dimethylpropyl, 2-methylsulfanylethyl, phenyl, benzyl, and pyridylmethyl; or
R¹ and R², taken together with the carbon atom to which they are attached, form a phenyl, which is substituted with 2 substituents selected from fluoro and CF₃;
R^{1a} is selected from bicyclo[1.1.1]pentanyl, cyclopropyl, 1-fluorocyclopropyl, 2,2-difluorocyclopropyl, 1-methylclopropyl, 1-fluorocyclobutyl, 3,3-difluorocyclobutyl, phenyl, isoxazolyl, methyl, ethyl, propyl, isopropyl, tert-butyl, CHFCl, CHCl₂, CHF₂, CF₃, 1-fluoro-1-methyl-ethyl, 2,2-difluoroethyl, 2,2,2-trifluoroethyl, 1-fluoroethyl, and 1,1,2,2,2-pentachloroethyl; and
R⁸ and R⁹ are each independently selected from hydrogen, fluoro and CF₃.

In a preferred embodiment, the present invention provides a compound of formula (I) as described herein, or a pharmaceutically acceptable salt thereof, wherein:
R¹ is a group

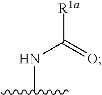

R² is selected from C₃-C₁₀-cycloalkyl and C₁-C₆-alkyl; and
R^{1a} is selected from C₃-C₁₀-cycloalkyl, C₁-C₆-alkyl and halo-C₁-C₆-alkyl, wherein said C₃-C₁₀-cycloalkyl is optionally substituted with 1 to 2 halogen substituents.

In a particularly preferred embodiment, the present invention provides a compound of formula (I) as described herein, or a pharmaceutically acceptable salt thereof, wherein:
R¹ is a group

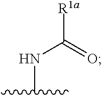

R² is selected from cyclopropyl, 1,1-dimethylpropyl, 1-ethylpropyl, sec-butyl and tert-butyl; and
R^{1a} is selected from cyclopropyl, 1-fluorocyclopropyl, 2,2-difluorocyclopropyl, isopropyl, tert-butyl, 1-fluoroethyl, CHFCl, CF₃, and CHF₂.

In a preferred embodiment, the present invention provides a compound of formula (I) as described herein, or a pharmaceutically acceptable salt thereof, wherein R¹ is a group

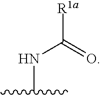

In one embodiment, the present invention provides a compound of formula (I) as described herein, or a pharmaceutically acceptable salt thereof, wherein:
R¹ is selected from a group

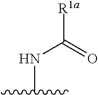

and C₆-C₁₄-aryloxy substituted with 1 to 2 halogen substituents; and
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl and halo-C₁-C₆-alkyl.

In one embodiment, the present invention provides a compound of formula (I) as described herein, or a pharmaceutically acceptable salt thereof, wherein:
R¹ is a group

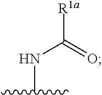

and
R^{1a} is selected from C₃-C₁₀-cycloalkyl, 3- to 14-membered heteroaryl, C₁-C₆-alkyl, and halo-C₁-C₆-alkyl, wherein said 3- to 14-membered heteroaryl is substituted with 1 halo-C₁-C₆-alkyl substituent.

In a preferred embodiment, the present invention provides a compound of formula (I) as described herein, or a pharmaceutically acceptable salt thereof, wherein:
R¹ is a group

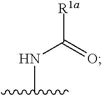

and
R^{1a} is selected from C₃-C₁₀-cycloalkyl, C₁-C₆-alkyl and halo-C₁-C₆-alkyl, wherein said C₃-C₁₀-cycloalkyl is substituted with 1-2 halogen substituents.

In a preferred embodiment, the present invention provides a compound of formula (I) as described herein, or a pharmaceutically acceptable salt thereof, wherein:
R¹ is a group

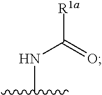

and
R^{1a} is selected from C₃-C₁₀-cycloalkyl, C₁-C₆-alkyl and halo-C₁-C₆-alkyl, wherein said C₃-C₁₀-cycloalkyl is substituted with 1 halogen substituent.

In a preferred embodiment, the present invention provides a compound of formula (I) as described herein, or a pharmaceutically acceptable salt thereof, wherein:
R¹ is a group

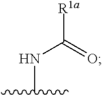

and
R^{1a} is selected from C₁-C₆-alkyl and halo-C₁-C₆-alkyl.

In a particularly preferred embodiment, the present invention provides a compound of formula (I) as described herein, or a pharmaceutically acceptable salt thereof, wherein:
R¹ is a group

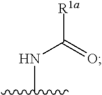

and
R^{1a} is selected from 2,2-difluorocyclopropyl, 1-fluorocyclopropyl, isopropyl, tert-butyl, CF₃, and CHF₂.

In a particularly preferred embodiment, the present invention provides a compound of formula (I) as described herein, or a pharmaceutically acceptable salt thereof, wherein:
R¹ is a group

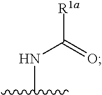

and
R^{1a} is selected from 1-fluorocyclopropyl, isopropyl, tert-butyl, CF₃, and CHF₂.

In a particularly preferred embodiment, the present invention provides a compound of formula (I) as described herein, or a pharmaceutically acceptable salt thereof, wherein:
R¹ is a group

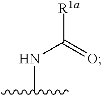

and
R^{1a} is selected from isopropyl, tert-butyl, CF₃, and CHF₂.

In a preferred embodiment, the present invention provides a compound of formula (I) as described herein, or a pharmaceutically acceptable salt thereof, wherein
R¹ is a group

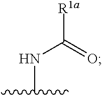

and
R^{1a} is halo-C₁-C₆-alkyl.

In a particularly preferred embodiment, the present invention provides a compound of formula (I) as described herein, or a pharmaceutically acceptable salt thereof, wherein
R¹ is a group

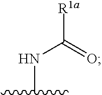

and
R^{1a} is selected from CHF₂ and CF₃.

In a particularly preferred embodiment, the present invention provides a compound of formula (I) as described herein, or a pharmaceutically acceptable salt thereof, wherein
R¹ is a group

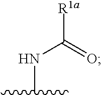

and
R^{1a} is CF₃.

In a particularly preferred embodiment, the present invention provides a compound of formula (I) as described herein, or a pharmaceutically acceptable salt thereof, wherein
R¹ is a group

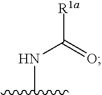

and
R^{1a} is CHF₂.

In a preferred embodiment, the present invention provides a compound of formula (I) as described herein, or a pharmaceutically acceptable salt thereof, wherein:
R¹ is a group

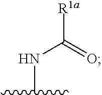

and
R^{1a} is C₃-C₁₀-cycloalkyl, wherein said C₃-C₁₀-cycloalkyl is substituted with 1-2 halogen substituents.

In a particularly preferred embodiment, the present invention provides a compound of formula (I) as described herein, or a pharmaceutically acceptable salt thereof, wherein:
R¹ is a group

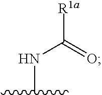

and
R^{1a} is selected from 2,2-difluorocyclopropyl and 1-fluorocyclopropyl.

In a particularly preferred embodiment, the present invention provides a compound of formula (I) as described herein, or a pharmaceutically acceptable salt thereof, wherein:
R¹ is a group

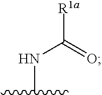

and
R^{1a} is 2,2-difluorocyclopropyl.

In a particularly preferred embodiment, the present invention provides a compound of formula (I) as described herein, or a pharmaceutically acceptable salt thereof, wherein:
R¹ is a group

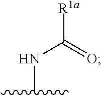

and
R^{1a} is 1-fluorocyclopropyl.

In one embodiment, the present invention provides a compound of formula (I) as described herein, or a pharmaceutically acceptable salt thereof, wherein R² is selected from hydrogen, C₃-C₁₀-cycloalkyl, and C₁-C₆-alkyl.

In one embodiment, the present invention provides a compound of formula (I) as described herein, or a pharmaceutically acceptable salt thereof, wherein R² is selected from hydrogen, C₃-C₁₀-cycloalkyl, C₃-C₁₀-cycloalkyl-C₁-C₆-alkyl, (3- to 14-membered heteroaryl)-C₁-C₆-alkyl and C₁-C₆-alkyl, wherein said C₃-C₁₀-cycloalkyl is optionally substituted with 1 C₁-C₆-alkyl substituent.

In a preferred embodiment, the present invention provides a compound of formula (I) as described herein, or a pharmaceutically acceptable salt thereof, wherein R² is selected from C₃-C₁₀-cycloalkyl and C₁-C₆-alkyl.

In a particularly preferred embodiment, the present invention provides a compound of formula (I) as described herein, or a pharmaceutically acceptable salt thereof, wherein R² is selected from cyclopropyl and tert-butyl.

In a particularly preferred embodiment, the present invention provides a compound of formula (I) as described herein, or a pharmaceutically acceptable salt thereof, wherein R² is selected from cyclopropyl, sec-butyl and tert-butyl. In a preferred embodiment, the present invention provides a compound of formula (I) as described herein, or a pharmaceutically acceptable salt thereof, wherein R² is C₁-C₆-alkyl.

In a particularly preferred embodiment, the present invention provides a compound of formula (I) as described herein, or a pharmaceutically acceptable salt thereof, wherein R² is tert-butyl.

In one embodiment, the present invention provides a compound of formula (I) as described herein, or a pharmaceutically acceptable salt thereof, wherein
R^{3a} is C₁-C₆-alkyl; R^{3b} is hydrogen or C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 1 to 2 C₁-C₆-alkyl substituents.

In a preferred embodiment, the present invention provides a compound of formula (I) as described herein, or a pharmaceutically acceptable salt thereof, wherein
R^{3a} and R^{3b} are both C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 2 C₁-C₆-alkyl substituents.

In a preferred embodiment, the present invention provides a compound of formula (I) as described herein, or a pharmaceutically acceptable salt thereof, wherein:
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 2 C₁-C₆-alkyl substituents.

In a particularly preferred embodiment, the present invention provides a compound of formula (I) as described herein, or a pharmaceutically acceptable salt thereof, wherein
R^{3a} and R^{3b} are both methyl; and R^{4a} and R^{4b} are both hydrogen; or
R^{3a} and R^{3b}, taken together with the carbon atom to which they are attached, form a cyclopropyl; and R^{4a} and R^{4b} are both hydrogen; or
R^{3a} and R^{4a}, taken together with the carbon atoms to which they are attached, form a cyclopropyl or a cyclopentyl; and R^{3b} and R^{4b} are both hydrogen; wherein said cyclopropyl is substituted with 2 methyl substituents.

In a particularly preferred embodiment, the present invention provides a compound of formula (I) as described herein, or a pharmaceutically acceptable salt thereof, wherein
R^{3a} and R^{4a}, taken together with the carbon atoms to which they are attached, form a cyclopropyl or a cyclopentyl; and R^{3b} and R^{4b} are both hydrogen; wherein said cyclopropyl is substituted with 2 methyl substituents.

In a particularly preferred embodiment, the present invention provides a compound of formula (I) as described herein, or a pharmaceutically acceptable salt thereof, wherein the group

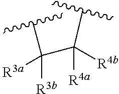

is selected from

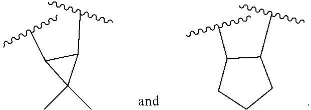

In a particularly preferred embodiment, the present invention provides a compound of formula (I) as described herein, or a pharmaceutically acceptable salt thereof, wherein the group

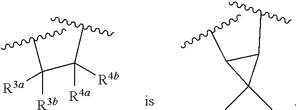

In a particularly preferred embodiment, the present invention provides a compound of formula (I) as described herein, or a pharmaceutically acceptable salt thereof, wherein the group

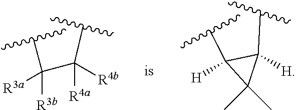

In a particularly preferred embodiment, the present invention provides a compound of formula (I) as described herein, or a pharmaceutically acceptable salt thereof, wherein the group

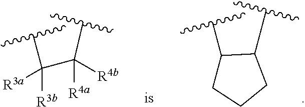

In a particularly preferred embodiment, the present invention provides a compound of formula (I) as described herein, or a pharmaceutically acceptable salt thereof, wherein the group

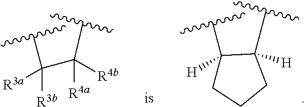

In one embodiment, the present invention provides a compound of formula (I) as described herein, or a pharmaceutically acceptable salt thereof, wherein L is C₁-C₃-alkyl.

In a preferred embodiment, the present invention provides a compound of formula (I) as described herein, or a pharmaceutically acceptable salt thereof, wherein L is CH₂ or CH₂CH₂.

In a particularly preferred embodiment, the present invention provides a compound of formula (I) as described herein, or a pharmaceutically acceptable salt thereof, wherein L is CH₂.

In one embodiment, the present invention provides a compound of formula (I) as described herein, or a pharmaceutically acceptable salt thereof, wherein:
L is C₁-C₆-alkyl; and
R⁵ is selected from 3- to 14-membered heterocycloalkyl and 3- to 14-membered heteroaryl; wherein said 3- to 14-membered heterocycloalkyl is substituted with 1 oxo substituent.

In a preferred embodiment, the present invention provides a compound of formula (I) as described herein, or a pharmaceutically acceptable salt thereof, wherein:
L is C₁-C₆-alkyl; and
R⁵ is 3- to 14-membered heterocycloalkyl substituted with 1 oxo substituent.

In a particularly preferred embodiment, the present invention provides a compound of formula (I) as described herein, or a pharmaceutically acceptable salt thereof, wherein:
L is CH₂; and
R⁵ is pyrrolidinyl substituted with 1 oxo substituent.

In one embodiment, the present invention provides a compound of formula (I) as described herein, or a pharmaceutically acceptable salt thereof, wherein R⁵ is a 3- to 9-membered heterocycloalkyl substituted with 1 oxo substituent.

In one embodiment, the present invention provides a compound of formula (I) as described herein, or a pharmaceutically acceptable salt thereof, wherein R⁵ is a 3- to 8-membered heterocycloalkyl substituted with 1 oxo substituent.

In one embodiment, the present invention provides a compound of formula (I) as described herein, or a pharmaceutically acceptable salt thereof, wherein R⁵ is a 3- to 7-membered heterocycloalkyl substituted with 1 oxo substituent.

In one embodiment, the present invention provides a compound of formula (I) as described herein, or a pharmaceutically acceptable salt thereof, wherein R⁵ is a 3- to 6-membered heterocycloalkyl substituted with 1 oxo substituent.

In one embodiment, the present invention provides a compound of formula (I) as described herein, or a pharmaceutically acceptable salt thereof, wherein R⁵ is a 4- to 6-membered heterocycloalkyl substituted with 1 oxo substituent.

In a preferred embodiment, the present invention provides a compound of formula (I) as described herein, or a pharmaceutically acceptable salt thereof, wherein R⁵ is a 5- to 6-membered heterocycloalkyl substituted with 1 oxo substituent.

In a particularly preferred embodiment, the present invention provides a compound of formula (I) as described herein, or a pharmaceutically acceptable salt thereof, wherein R⁵ is selected from pyrrolidinyl and piperidyl, each of which is substituted with 1 oxo substituent.

In a particularly preferred embodiment, the present invention provides a compound of formula (I) as described herein, or a pharmaceutically acceptable salt thereof, wherein R⁵ is pyrrolidinyl substituted with 1 oxo substituent.

In a particularly preferred embodiment, the present invention provides a compound of formula (I) as described herein, or a pharmaceutically acceptable salt thereof, wherein R⁵ is piperidyl substituted with 1 oxo substituent.

In a particularly preferred embodiment, the present invention provides a compound of formula (I) as described herein, or a pharmaceutically acceptable salt thereof, wherein R⁵ is

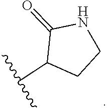 [SMILES: *C1CCNC1=O]

In a particularly preferred embodiment, the present invention provides a compound of formula (I) as described herein, or a pharmaceutically acceptable salt thereof, wherein R⁵ is

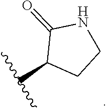 [SMILES: *[C@@H]1CCNC1=O]

In a particularly preferred embodiment, the present invention provides a compound of formula (I) as described herein, or a pharmaceutically acceptable salt thereof, wherein R⁵ is

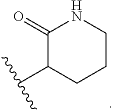 [SMILES: *C1CCCNC1=O]

In a particularly preferred embodiment, the present invention provides a compound of formula (I) as described herein, or a pharmaceutically acceptable salt thereof, wherein R⁵ is

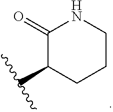 [SMILES: *[C@@H]1CCCNC1=O]

In one embodiment, the present invention provides a compound of formula (I) as described herein, or a pharmaceutically acceptable salt thereof, wherein:
R⁶ is selected from fluoro and chloro; and
R⁷ is selected from hydrogen and chloro.

In a preferred embodiment, the present invention provides a compound of formula (I) as described herein, or a pharmaceutically acceptable salt thereof, wherein:
R⁶ is fluoro; and
R⁷ is chloro.

In one embodiment, the present invention provides a compound of formula (I) as described herein, or a pharmaceutically acceptable salt thereof, wherein:
L is C₁-C₆-alkyl;
R¹ is selected from a group

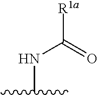

and C₆-C₁₄-aryloxy substituted with 1 to 2 halogen substituents;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl halo-C₁-C₆-alkyl;
R² is selected from hydrogen, C₃-C₁₀-cycloalkyl, C₃-C₁₀-cycloalkyl-C₁-C₆-alkyl, (3- to 14-membered heteroaryl)-C₁-C₆-alkyl and C₁-C₆-alkyl, wherein said C₃-C₁₀-cycloalkyl is optionally substituted with 1 C₁-C₆-alkyl substituent;
R^{3a} is C₁-C₆-alkyl; R^{3b} is hydrogen or C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 1 to 2 C₁-C₆-alkyl substituents;
R⁵ is selected from 3- to 14-membered heterocycloalkyl and 3- to 14-membered heteroaryl; wherein said 3- to 14-membered heterocycloalkyl is substituted with 1 oxo substituent;
R⁶ is selected from fluoro and chloro; and
R⁷ is selected from hydrogen and chloro.

In a preferred embodiment, the present invention provides a compound of formula (I) as described herein, or a pharmaceutically acceptable salt thereof, wherein:
L is C₁-C₆-alkyl;
R¹ is a group

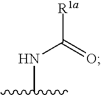

R^{1a} is selected from C₃-C₁₀-cycloalkyl, C₁-C₆-alkyl and halo-C₁-C₆-alkyl, wherein said C₃-C₁₀-cycloalkyl is substituted with 1 halogen substituent;
R² is selected from C₃-C₁₀-cycloalkyl and C₁-C₆-alkyl;
R^{3a} and R^{3b} are both C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 2 C₁-C₆-alkyl substituents;
R⁵ is 3- to 14-membered heterocycloalkyl substituted with 1 oxo substituent;
R⁶ is fluoro; and
R⁷ is chloro.

In a particularly preferred embodiment, the present invention provides a compound of formula (I) as described herein, or a pharmaceutically acceptable salt thereof, wherein:
L is CH₂;
R¹ is a group

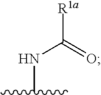

R^{1a} is selected from 1-fluorocyclopropyl, isopropyl, tert-butyl, CF₃, and CHF₂;
R² is selected from cyclopropyl, sec-butyl and tert-butyl;
R^{3a} and R^{3b} are both methyl; and R^{4a} and R^{4b} are both hydrogen; or
R^{3a} and R^{3b}, taken together with the carbon atom to which they are attached, form a cyclopropyl; and R^{4a} and R^{4b} are both hydrogen; or
R^{3a} and R^{4a}, taken together with the carbon atoms to which they are attached, form a cyclopropyl or a cyclopentyl; and R^{3b} and R^{4b} are both hydrogen; wherein said cyclopropyl is substituted with 2 methyl substituents;
R⁵ is pyrrolidinyl substituted with 1 oxo substituent;
R⁶ is fluoro; and
R⁷ is chloro.

In one embodiment, the present invention provides a compound of formula (I) as described herein, or a pharmaceutically acceptable salt thereof, wherein:
L is C₁-C₆-alkyl;
R¹ is a group

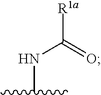

R^{1a} is selected from C₃-C₁₀-cycloalkyl, 3- to 14-membered heteroaryl, C₁-C₆-alkyl, and halo-C₁-C₆-alkyl, wherein said 3- to 14-membered heteroaryl is substituted with 1 halo-C₁-C₆-alkyl substituent;
R² is selected from hydrogen, C₃-C₁₀-cycloalkyl, and C₁-C₆-alkyl;
R^{3a} is C₁-C₆-alkyl; R^{3b} is hydrogen or C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 1 to 2 C₁-C₆-alkyl substituents;
R⁵ is selected from 3- to 14-membered heterocycloalkyl and 3- to 14-membered heteroaryl; wherein said 3- to 14-membered heterocycloalkyl is substituted with 1 oxo substituent;
R⁶ is selected from fluoro and chloro; and
R⁷ is selected from hydrogen and chloro.

In a preferred embodiment, the present invention provides a compound of formula (I) as described herein, or a pharmaceutically acceptable salt thereof, wherein:
L is C₁-C₆-alkyl;
R¹ is a group

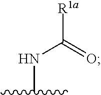

R^{1a} is selected from C₁-C₆-alkyl and halo-C₁-C₆-alkyl;
R² is selected from C₃-C₁₀-cycloalkyl and C₁-C₆-alkyl;
R^{3a} and R^{3b} are both C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 2 C₁-C₆-alkyl substituents;
R⁵ is 3- to 14-membered heterocycloalkyl substituted with 1 oxo substituent;
R⁶ is fluoro; and
R⁷ is chloro.

In a particularly preferred embodiment, the present invention provides a compound of formula (I) as described herein, or a pharmaceutically acceptable salt thereof, wherein:
L is CH₂;
R¹ is a group

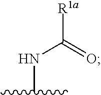

R^{1a} is selected from isopropyl, tert-butyl, CF₃, and CHF₂;
R² is selected from cyclopropyl and tert-butyl;
R^{3a} and R^{3b} are both methyl; and R^{4a} and R^{4b} are both hydrogen; or
R^{3a} and R^{3b}, taken together with the carbon atom to which they are attached, form a cyclopropyl; and R^{4a} and R^{4b} are both hydrogen; or
R^{3a} and R^{4a}, taken together with the carbon atoms to which they are attached, form a cyclopropyl or a cyclopentyl; and R^{3b} and R^{4b} are both hydrogen; wherein said cyclopropyl is substituted with 2 methyl substituents;
R⁵ is pyrrolidinyl substituted with 1 oxo substituent;
R⁶ is fluoro; and
R⁷ is chloro.

In one embodiment, the present invention provides a compound of formula (I) as described herein, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide;
N-[(1S)-1-[(1R,2S,5S)-2-[[but-2-ynoyl-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
(1R,2S,5S)—N′-[(2R)-2-chloro-2-fluoro-acetyl]-3-[2-(3,5-difluorophenoxy)acetyl]-6,6-dimethyl-N′—[[(3S)-2-oxopyrrolidin-3-yl]methyl]-3-azabicyclo[3.1.0]hexane-2-carbohydrazide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.2.0]heptane-3-carbonyl]-2,2-dimethyl-propyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-(1H-pyrazol-3-ylmethyl)amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-5-(trifluoromethyl)isoxazole-3-carboxamide;
N-[(1S)-1-[(1R,2S,5S)-2-[[(2-chloroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-phenyl-ethyl]-2,2,2-trifluoro-acetamide;
N-[(1S)-1-benzyl-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methylsulfanyl-propyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2-methyl-propanamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-(1-methylcyclopropyl)-2-oxo-ethyl]-2,2,2-trifluoro-acetamide;
N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-(4-pyridylmethyl)ethyl]-2,2,2-trifluoro-acetamide;
N-[(1S)-1-(1-bicyclo[1.1.1]pentanylmethyl)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
(3S,3aS,6aR)—N′-[(2R)-2-chloro-2-fluoro-acetyl]-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide;
(3R,3aR,6aS)—N′-[(2R)-2-chloro-2-fluoro-acetyl]-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide;
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-propanamide;
N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide;
N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-cyclobutyl-2-oxo-ethyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide;
N-[(1S)-1-[(2S,4S)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide;
N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-methyl-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]propanamide;
Rac-2,2-difluoro-N-[(1S,2S)-2-methyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-methyl-cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-methyl-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
Rac-2,2-difluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-methyl-propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dimethyl-propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3,3-trifluoro-propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]butanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-fluoro-2-methyl-propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-fluoro-2-methyl-propanamide;
Rac-2-fluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-dichloro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dichloro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,3,3,3-pentafluoro-propanamide;
Rac-2-fluoro-N-[(1S,2S)-2-methyl-1-[rac-(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]propanamide;
N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-TH-cyclopenta[c]pyrrole-2-carbonyl]-2-methyl-butyl]-2,2-difluoro-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,3,3,3-pentafluoro-propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-TH-cyclopenta[c]pyrrole-2-carbonyl]-2-ethyl-butyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,3,3,3-pentafluoro-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-propanamide;
Rac-2-fluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,3,3,3-pentafluoro-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
2,2-dichloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]acetamide;
Rac-2,2-difluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-dimethyl-propanamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]benzamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclobutanecarboxamide;
(1S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide;
(1S)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1R)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclobutanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-3,3-difluoro-cyclobutanecarboxamide;
(1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1R)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide;
(1R)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide;
2,2-difluoro-N-[rac-(1S,2S)-2-methyl-1-[rac-(1R,2S,5S)-6,6-dimethyl-2-[[[rac-(2S)-2-chloro-2-fluoro-acetyl]-[[rac-(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
2,2-difluoro-N-[rac-(1S,2R)-2-methyl-1-[rac-(1R,2S,5S)-6,6-dimethyl-2-[[[rac-(2R)-2-chloro-2-fluoro-acetyl]-[[rac-(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2R)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
(2S)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide;
N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2S)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
(2R)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
rac-2,2-difluoro-N-[(1S,2R)-2-methyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclobutanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide;
N-[(1S,2S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-3,3-difluoro-cyclobutanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2-difluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-3,3-difluoro-cyclobutanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
rac-2-fluoro-N-[(1S)-2-ethyl-1-[(2S)-4,4-dimethyl-2-[[[(2R)-2-chloro-2-fluoro-acetyl]-[[(3S)-2-oxopyrrolidin-3-yl]methyl]amino]carbamoyl]pyrrolidine-1-carbonyl]butyl]propanamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-1-fluoro-cyclopropanecarboxamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-2,2-difluoro-acetamide;
(2S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide;
(2R)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide;
rac-2-fluoro-N-[(1S)-2,2-dimethyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-1-fluoro-cyclopropanecarboxamide;
(1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
(1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
N-[(1S)-1-[(3S,3aS,6aR)-3-[I[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-butyl]-1-fluoro-cyclopropanecarboxamide;
N-[(1S)-1-[(2S)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide;
N-[(1S)-1-[(2R)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide;
N-[(1S)-2-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide;
(1S)—N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide;
(1S)—N-[(1S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide;
N-[(1S,2S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide;
N-[(1S,2S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide;
N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide;
N-[(1S)-1-[(2S)-2-[[[(2R)-2-chloro-2-fluoro-acetyl]-[[(3S)-2-oxo-3-piperidyl]methyl]amino]carbamoyl]-4,4-dimethyl-pyrrolidine-1-carbonyl]-2,2-dimethyl-butyl]cyclopropanecarboxamide;
N-[(1S)-1-[(2S)-2-[[[(2R)-2-chloro-2-fluoro-acetyl]-[[(3R)-2-oxo-3-piperidyl]methyl]amino]carbamoyl]-4,4-dimethyl-pyrrolidine-1-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide;
N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide;
N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide;
N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2-difluoro-acetamide;
(1R,2S,5S)-3-[3,5-bis(trifluoromethyl)benzoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide;
(1R,2S,5S)-3-[2-[3,5-bis(trifluoromethyl)phenyl]acetyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide;
(1R,2S,5S)—N′-[(2R)-2-chloro-2-fluoro-acetyl]-3-[3-fluoro-5-(trifluoromethyl)benzoyl]-6,6-dimethyl-N′—[[(3S)-2-oxopyrrolidin-3-yl]methyl]-3-azabicyclo[3.1.0]hexane-2-carbohydrazide;
(1R,2S,5S)-3-[(E)-3-[3,5-bis(trifluoromethyl)phenyl]prop-2-enoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide;
(1R,2S,5S)—N′-[(2R)-2-chloro-2-fluoro-acetyl]-3-[(E)-3-(3,5-difluorophenyl)prop-2-enoyl]-6,6-dimethyl-N′—[[(3S)-2-oxopyrrolidin-3-yl]methyl]-3-azabicyclo[3.1.0]hexane-2-carbohydrazide;
(3S,3aS,6aR)—N′-[(2R)-2-chloro-2-fluoro-acetyl]-2-[(2S)-2-[(6-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide;
(3S,3aS,6aR)—N′-[(2R)-2-chloro-2-fluoro-acetyl]-2-[(2S)-2-[(5-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide;
(3S,3aS,6aR)—N′-[(2R)-2-chloro-2-fluoro-acetyl]-2-[(2S)-3,3-dimethyl-2-(2-pyridylamino)butanoyl]-N′—[[(3S)-2-oxopyrrolidin-3-yl]methyl]-3,3a,4,5,6,6a-hexahydro-1H-cyclopenta[c]pyrrole-3-carbohydrazide;
(2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide; and
(2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide.

In one embodiment, the present invention provides a compound of formula (I) as described herein, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.2.0]heptane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-(1H-pyrazol-3-ylmethyl)amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
(1R,2S,5S)—N′-[(2R)-2-chloro-2-fluoro-acetyl]-3-[2-(3,5-difluorophenoxy)acetyl]-6,6-dimethyl-N′—[[(3S)-2-oxopyrrolidin-3-yl]methyl]-3-azabicyclo[3.1.0]hexane-2-carbohydrazide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-5-(trifluoromethyl)isoxazole-3-carboxamide;
3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide;
3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]-N-methyl-propanamide;
3-[[(2R)-2-chloro-2-fluoro-acetyl]-[[(1R,2S,5S)-3-[(2S)-2-[(2,2-difluoroacetyl)amino]-3,3-dimethyl-butanoyl]-6,6-dimethyl-3-azabicyclo[3.1.0]hexane-2-carbonyl]amino]amino]propanamide;
3-[[(2R)-2-chloro-2-fluoro-acetyl]-[[(1R,2S,5S)-3-[(2S)-2-[(2,2-difluoroacetyl)amino]-3,3-dimethyl-butanoyl]-6,6-dimethyl-3-azabicyclo[3.1.0]hexane-2-carbonyl]amino]amino]-N-methyl-propanamide;
N-[(1S)-1-[(1R,2S,5S)-2-[[(2-chloroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
3-[(2-chloroacetyl)-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide;
2,2,2-trifluoro-N-[(1S)-1-[(1R,2S,5S)-2-[[(2-fluoroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]acetamide;
3-[[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-(2-fluoroacetyl)amino]propanamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-phenyl-ethyl]-2,2,2-trifluoro-acetamide;
N-[(1S)-1-benzyl-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methylsulfanyl-propyl]-2,2,2-trifluoro-acetamide;
N-[(1S)-1-[(1R,2S,5S)-2-[[(2-chloro-3-oxo-butanoyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(2S)-2-[[(2-chloro-2-fluoro-acetyl)-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide;
N-[(1S)-1-[(4S)-4-[[(2-chloro-2-fluoro-acetyl)-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-(1-methylcyclopropyl)-2-oxo-ethyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2-methyl-propanamide;
N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2,2-trifluoro-acetamide;
N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2-difluoro-acetamide;
N-[(1S)-1-(1-bicyclo[1.1.1]pentanylmethyl)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-(4-pyridylmethyl)ethyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-difluoro-pyrrolidine-1-carbonyl]-2,2-dimethyl-propyl]-2,2,2-trifluoro-acetamide;
N-[(1S)-1-(1-bicyclo[1.1.1]pentanyl)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
(1R,2S,5S)—N′-[(2R)-2-chloro-2-fluoro-acetyl]-6,6-dimethyl-N′—[[(3S)-2-oxopyrrolidin-3-yl]methyl]-3-[3-(trifluoromethyl)isoxazole-5-carbonyl]-3-azabicyclo[3.1.0]hexane-2-carbohydrazide;
N-[(1S)-1-[(2S,3S)-2-[[[(2R)-2-chloro-2-fluoro-acetyl]-[[(3S)-2-oxopyrrolidin-3-yl]methyl]amino]carbamoyl]-3-methyl-pyrrolidine-1-carbonyl]-2,2-dimethyl-propyl]-2,2,2-trifluoro-acetamide;
N-[(1S)-1-[(2S,3S)-2-[[[(2R)-2-chloro-2-fluoro-acetyl]-[[(3S)-2-oxopyrrolidin-3-yl]methyl]amino]carbamoyl]-3-(trifluoromethyl)pyrrolidine-1-carbonyl]-2,2-dimethyl-propyl]-2,2,2-trifluoro-acetamide; and
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2,2-trifluoro-acetamide.

In one embodiment, the present invention provides a compound of formula (I) as described herein, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.2.0]heptane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-(1H-pyrazol-3-ylmethyl)amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
(1R,2S,5S)—N′-[(2R)-2-chloro-2-fluoro-acetyl]-3-[2-(3,5-difluorophenoxy)acetyl]-6,6-dimethyl-N′—[[(3S)-2-oxopyrrolidin-3-yl]methyl]-3-azabicyclo[3.1.0]hexane-2-carbohydrazide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-5-(trifluoromethyl)isoxazole-3-carboxamide; and
N-[(1S)-1-[(1R,2S,5S)-2-[[(2-chloroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide.
In a preferred embodiment, the present invention provides a compound of formula (I) as described herein, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-TH-cyclopenta[c]pyrrole-2-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2R)—N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2S)—N-[(1S,2S)-1-[(3S,3aS,6aR)-3-[[[(2S)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2R)-2-chloro-N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(1S)—N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide; and
(1S)—N-[(1S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide.

In a preferred embodiment, the present invention provides a compound of formula (I) as described herein, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
(1S)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1R)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2S)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2-difluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
(2S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
(1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
(1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
(2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide;
(2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide; and
(2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide.

In a particularly preferred embodiment, the present invention provides a compound of formula (I) as described herein, or a pharmaceutically acceptable salt thereof, wherein said compound is N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide.

In a particularly preferred embodiment, the present invention provides a compound of formula (I) as described herein, or a pharmaceutically acceptable salt thereof, wherein said compound is N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide.

In a particularly preferred embodiment, the present invention provides a compound of formula (I) as described herein, or a pharmaceutically acceptable salt thereof, wherein said compound is N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide.

In a particularly preferred embodiment, the present invention provides a compound of formula (I) as described herein, or a pharmaceutically acceptable salt thereof, wherein said compound is N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide.

In a particularly preferred embodiment, the present invention provides a compound of formula (I) as described herein, or a pharmaceutically acceptable salt thereof, wherein said compound is N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide.

In a particularly preferred embodiment, the present invention provides a compound of formula (I) as described herein, or a pharmaceutically acceptable salt thereof, wherein said compound is N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide.

In a particularly preferred embodiment, the present invention provides a compound of formula (I) as described herein, or a pharmaceutically acceptable salt thereof, wherein said compound is N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide.

In a particularly preferred embodiment, the present invention provides a compound of formula (I) as described herein, or a pharmaceutically acceptable salt thereof, wherein said compound is N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide.

In a particularly preferred embodiment, the present invention provides a compound of formula (I) as described herein, or a pharmaceutically acceptable salt thereof, wherein said compound is N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide.

In a particularly preferred embodiment, the present invention provides a compound of formula (I) as described herein, or a pharmaceutically acceptable salt thereof, wherein said compound is N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide.

In a particularly preferred embodiment, the present invention provides a compound of formula (I) as described herein, or a pharmaceutically acceptable salt thereof, wherein said compound is N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide.

In a particularly preferred embodiment, the present invention provides a compound of formula (I) as described herein, or a pharmaceutically acceptable salt thereof, wherein said compound is N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide.

In a particularly preferred embodiment, the present invention provides a compound of formula (I) as described herein, or a pharmaceutically acceptable salt thereof, wherein said compound is (2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide.

In a particularly preferred embodiment, the present invention provides a compound of formula (I) as described herein, or a pharmaceutically acceptable salt thereof, wherein said compound is (2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide.

In a particularly preferred embodiment, the present invention provides a compound of formula (I) as described herein, or a pharmaceutically acceptable salt thereof, wherein said compound is (2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-TH-cyclopenta[c]pyrrole-2-carbonyl]-2-methyl-butyl]-2-chloro-2-fluoro-acetamide.

In a particularly preferred embodiment, the present invention provides a compound of formula (I) as described herein, or a pharmaceutically acceptable salt thereof, wherein said compound is (2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-TH-cyclopenta[c]pyrrole-2-carbonyl]-2-methyl-butyl]-2-chloro-2-fluoro-acetamide.

In a particular embodiment, the present invention provides pharmaceutically acceptable salts of the compounds according to formula (I) as described herein. In a further particular embodiment, the present invention provides compounds according to formula (I) as described herein in their free form (i.e., as free bases or acids).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

All substituents, in particular, R¹ to R⁷, R^{1a}, R^{3a}, R^{3b}, R^{4a}, R^{4b} and L are as defined above and in the claims, unless otherwise indicated.

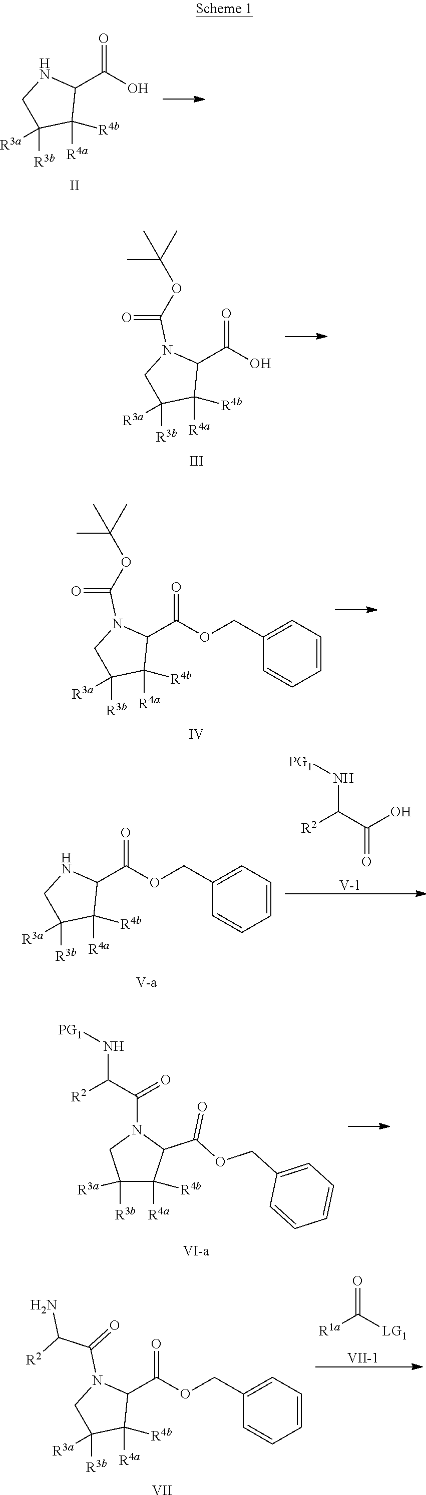

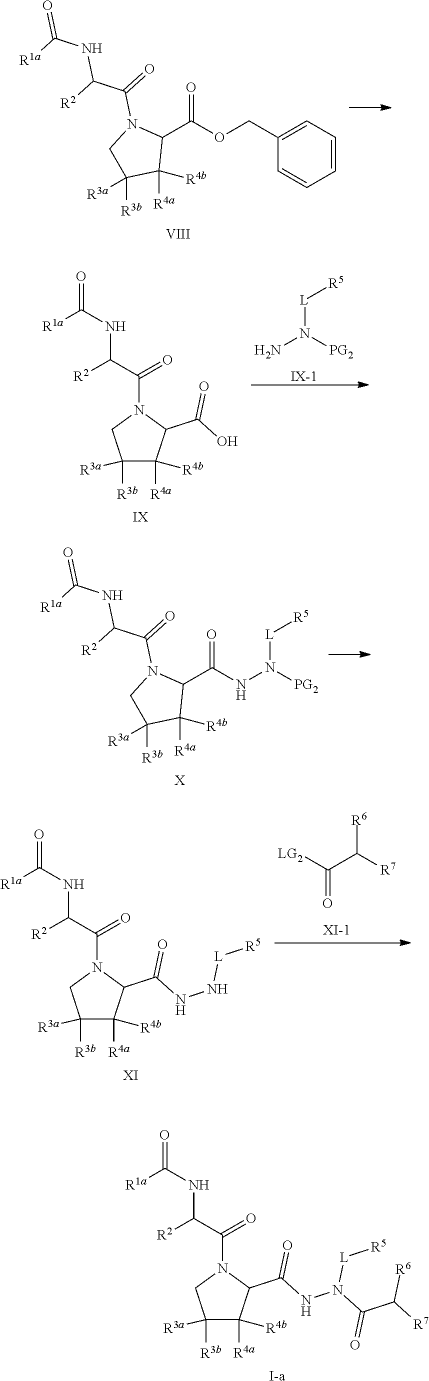

In Scheme 1, PG₁ and PG₂ are protective groups selected from Cbz and Boc, respectively;
LG₁ is Cl, OH, OEt or

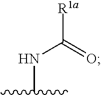

and LG₂ is halogen.

Compound of formula III can be prepared by a protection reaction of compound of formula II with di-tert-butyl dicarbonate in the presence of an organic base, such as TEA, DIPEA or DMAP, in a solvent such as DCM, THF, dioxane or a mixed solvent of dioxane and water. Then compound of formula III reacts with benzyl bromide in the presence of a base such as Na₂CO₃, K₂CO₃ or Cs₂CO₃, in a solvent such as DMF or CH₃CN, to afford compound of formula IV. Deprotection of compound of formula IV can afford compound of formula V-a in the presence of an acid such as HCl or TFA in a solvent such as DCM or dioxane or a neat reaction without any solvent. Compound of formula VI-a can be obtained by a coupling reaction using compound of formula V-1, compound of formula V-a, and coupling reagent(s), such as T₃P, HATU, HOPO, PyBOP or EDCI/HOBt, in the presence of an organic base, such TEA, DIEPA or DMAP. Deprotection of compound of formula VI-a in the presence of an acid such as HCl or TFA in a solvent such as DCM or dioxane, or a neat reaction without any solvent, can afford compound of formula VII. Compound of formula VIII can be obtained by a coupling reaction using compound of formula VII-1, compound of formula VII, and coupling reagent(s), such as T₃P, HATU, PyBOP, HOPO or EDCI/HOBt, in the presence of an organic base, such TEA, DIEPA or DMAP, in a solvent such as THF, EtOAc, DMF or DCM. Alternatively, compound of formula VIII can be obtained by a reaction of compound of formula VII and compound of formula VII-1 in the presence of an organic base such as TEA, DIEPA or DMAP and in a solvent such as MeOH, DCM, THF or DMF. Hydrogenolysis of compound of formula VIII in the presence of Pd/C, Pd(OH)₂ or a mixture of Pd/C and Pd(OH)₂, and in a solvent such as MeOH can afford compound of formula IX. Compound of formula IX reacts with compound of formula IX-1 in the presence of a coupling reagent, such as T₃P, HATU, PyBOP, HOPO or EDCI/HOBt, and an organic base such as TEA, DIPEA or DMAP, in a solvent such as THF, EtOAc, DMF or DCM, to afford compound of formula X. Deprotection of compound of formula X in the presence of an acid such as HCl or TFA, and (or not) in a solvent such as DCM or dioxane can afford compound of formula XI. Compound of formula XI then reacts with compound of formula XI-1 in the presence of coupling reagent(s), such as T₃P, HATU, PyBOP, HOPO or EDCI/HOBt, and a base such as TEA, DIPEA or DMAP, in a solvent such as THF, EtOAc, DMF or DCM, to afford compound of formula I-a.

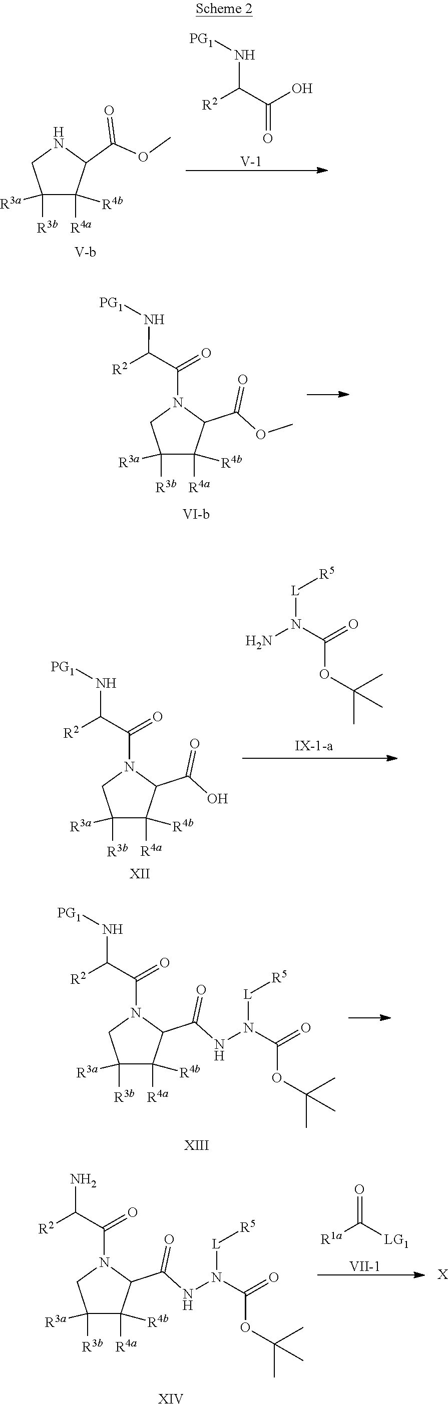

In Scheme 2, PG₁ and LG₁ are as defined in Scheme 1.

Alternatively, compound of formula X can be prepared by using compound of formula V-b as starting material. Compound of formula V-b reacts with compound of formula V-1 in the presence of coupling reagent(s), such as T₃P, HATU, PyBOP, HOPO or EDCI/HOBt, and an organic base such as TEA, DIPEA or DMAP, in a solvent such as DMF or DCM, to afford compound of formula VI-b. Hydrolyzation of compound of formula VI-b in the presence of a base such as LiOH·H₂O, NaOH or KOH, and in a mixed solution of MeOH and H₂O can afford compound of formula XII. Compound of formula VIII can be obtained by a coupling reaction using compound of formula XII, compound of formula IX-1-a, and coupling reagent(s), such as T₃P, HATU, HOPO, PyBOP or EDCI/HOBt, in the presence of an organic base, such as TEA, DIEPA or DMAP, in a solvent such as THF, EtOAc, DMF or DCM. Hydrogenolysis of compound of formula XIII in the presence of Pd/C, Pd(OH)₂ or a mixture of Pd/C and Pd(OH)₂ in a solvent such as MeOH can afford compound of formula XIV. Compound of formula XIV reacts with compound of formula VII-1 in the presence of coupling reagent(s), such as T₃P, HATU, PyBOP, HOPO or EDCI/HOBt, and an organic base such as TEA, DIPEA or DMAP, in a solvent such as DMF or DCM, to afford compound of formula X. Alternatively, compound of formula X can be obtained by a reaction of compound of formula XIV and compound of formula VII-1 in the presence of an organic base such as TEA, DIEPA or DMAP and in a solvent such as MeOH, DCM, THF or DMF. After deprotection and a coupling reaction, compound of formula I-a can be prepared from compound of formula X by following the last two steps of Scheme 1.

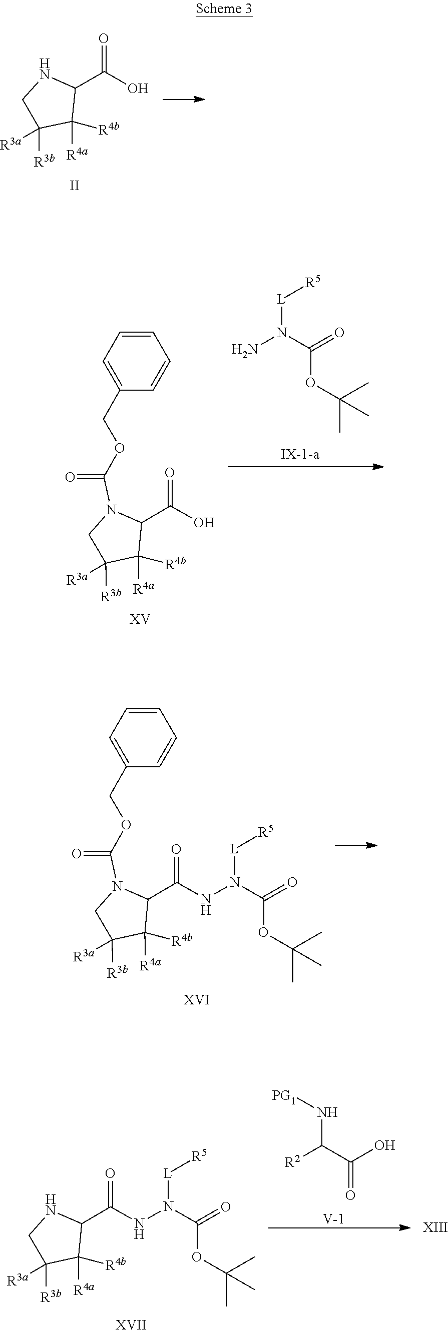

In Scheme 3, PG₁ is as defined in Scheme 1.

Alternatively, compound of formula I-a can also be prepared by Scheme 3. Compound of formula II reacts with benzyl carbonochloridate or benzyl carbonochloridate in the presence of an organic base, such as TEA, DIPEA or DMAP, in a solvent such as DCM, THF, dioxane or a mixed solvent of dioxane and water, to afford compound of formula XV. Compound of formula XV reacts with compound of formula IX-1-a in the presence of a coupling reagent, such as T₃P, HATU, PyBOP, HOPO or EDCI/HOBt, and an organic base such as TEA, DIPEA or DMAP, in a solvent such as THF, EtOAc, DMF or DCM, to afford compound of formula XVI. Hydrogenolysis of compound of formula XVI in the presence of Pd/C, Pd(OH)₂ or a mixture of Pd/C and Pd(OH)₂ in a solvent such as MeOH, EtOH or THF can afford compound of formula XVII. Compound of formula XVII reacts with compound of formula V-1 in presence of a coupling reagent(s), such as T₃P, HATU, HOPO, PyBOP or EDCI/HOBt and an organic base, such TEA, DIEPA or DMAP, in a solvent such as THF, EtOAc, DMF or DCM to afford compound formula XIII. By following the last four steps of Scheme 1, compound of formula I-a can be prepared from compound of formula XIII.

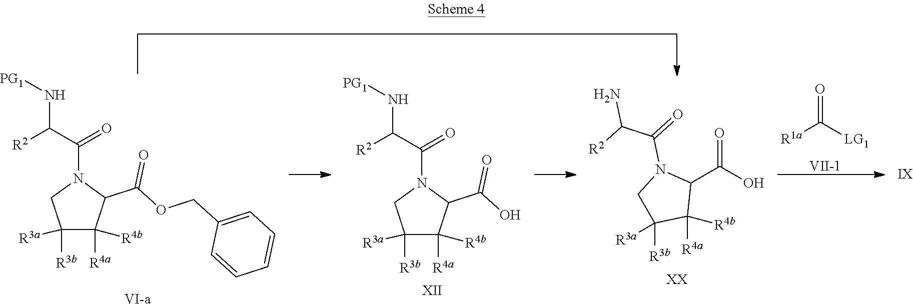

In Scheme 4, PG₁ is a protective group selected from Cbz or Boc; LG₁ is Cl, OEt or

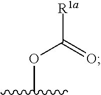

Alternatively, compound of formula IX can be prepared by using compound of formula VI-a as starting material. Hydrogenolysis of compound of formula VI-a in the presence of Pd/C, Pd(OH)₂ or a mixture of Pd/C and Pd(OH)₂ in a solvent such as MeOH, EtOH or THF can afford compound of formula XII. Then hydrolyzation of compound of formula XII in the presence of a base such as LiOH·H₂O, NaOH or KOH, and in a mixed solution of MeOH and H₂O can afford compound of formula XX. When PG₁ is Cbz, compound of formula XX can obtained by a hydrogenation reaction of compound of formula VI-a in the presence of Pd/C, Pd(OH)₂ or a mixture of Pd/C and Pd(OH)₂ in a solvent such as MeOH, EtOH or THF. Compound of formula XX reacts with compound of formula VII-1 in the presence of an organic base such as TEA, DIPEA or DMAP, in a solvent such as MeOH, DMF or DCM, to afford compound of formula IX. By following the last three steps of Scheme 1, compound of formula I-a can be prepared from compound of formula IX.

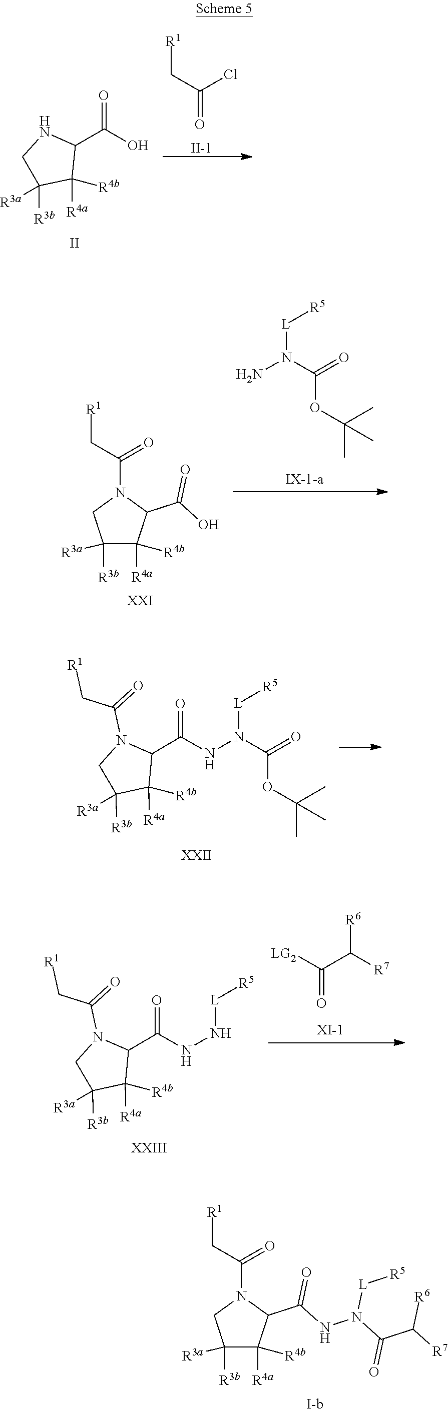

In Scheme 5, LG₂ is as defined in Scheme 1.

Compound of formula XXI can be prepared by a coupling reaction of compound of formula II with compound of formula II-1 in the presence of an organic base such as TEA, DIPEA or DMAP, in a solvent such as DMF or DCM. Compound of formula IX-1-a reacts with compound of formula XXI, in presence of coupling reagent(s), such as T₃P, HATU, PyBOP, HOPO or EDCI/HOBt, and an organic base, such as TEA, DIEPA or DMAP, in a solvent such as THF, EtOAc, DMF or DCM to afford compound formula XXII. Deprotection of compound formula XXII in the presence of an acid such as HCl or TFA in (or not) a solvent such as DCM or dioxane can afford compound of formula XXIII. Compound of formula XXIII then reacts with compound of formula XI-1 in the presence of coupling reagent(s), such as T₃P. HATU, PyBOP. HOPO or EDCI/HOBt. and a base such as TEA, DIPEA or DMAP, in a solvent such as in a solvent such as THF, EtOAc, DMF or DCM, to afford compound of formula I-b.

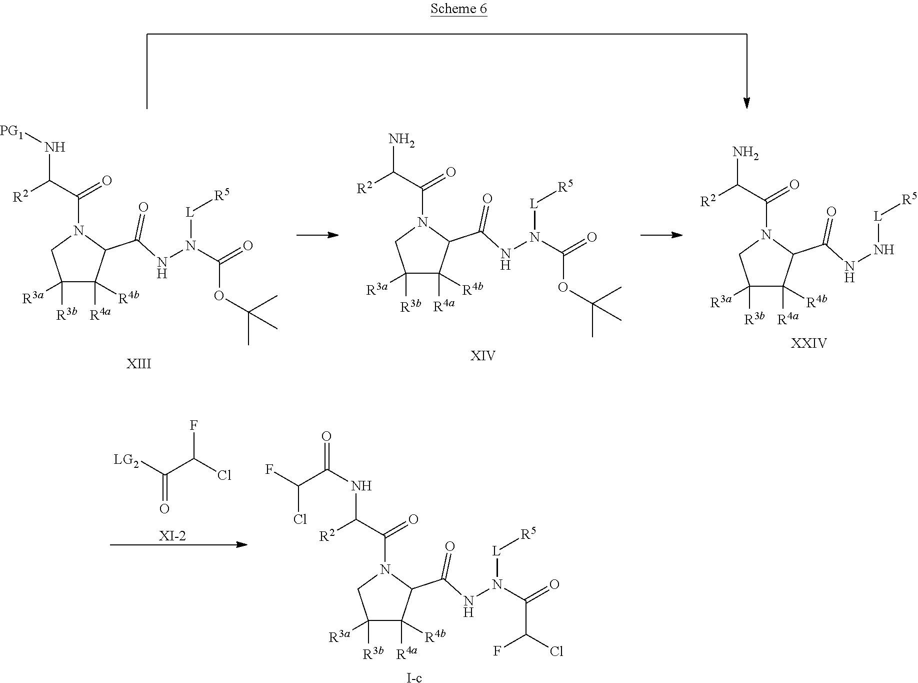

In Scheme 6, PG₁ and LG₂ is as defined in Scheme 1.

Compound of formula XIV can be obtained by a hydrogenation reaction of compound of formula XIII in the presence of Pd/C, Pd(OH)₂ or a mixture of Pd/C and Pd(OH)₂ in a solvent such as MeOH, EtOH or THF. Then compound of formula XIV is deprotected in the presence of an acid such as HCl or TFA in DCM, dioxane or without any solvent to afford compound formula XXIV. When PG₁ is Boc, compound of formula XXIV can also be obtained from compound of formula XIII in the same acid condition above mentioned. Compound of formula XXIV reacts with compound of formula XI-2 in the presence of coupling reagent(s), such as T₃P, HATU, PyBOP, HOPO, EDCI/HOBt or none, and a base such as TEA, DIPEA or DMAP, in a solvent such as THF, EtOAc, DMF or DCM, to afford compound of formula I-c.

In one aspect, the present invention provides a process of manufacturing a compound of formula (I) described herein, or a pharmaceutically acceptable salt thereof, comprising:
(a) reacting a compound of formula (XI)

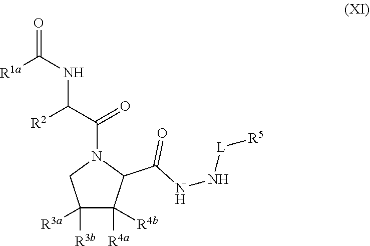

wherein R^{1a}, R², R^{3a}, R^{3b}, R^{4a}, R^{4b}, R⁵, and L are as defined herein;
with a compound of formula (XI-1),

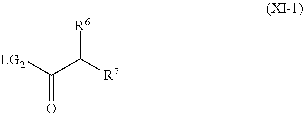

wherein R⁶ and R⁷ are as defined herein and LG₂ is a leaving group;
in the presence of a coupling reagent and a base;
to form a compound of formula (I-a)

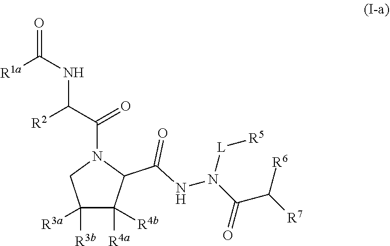

wherein R^{1a}, R², R^{3a}, R^{3b}, R^{4a}, R^{4b}, R⁵, R⁶, R⁷, and L are as defined herein; or
(b) reacting a compound of formula (XXIII)

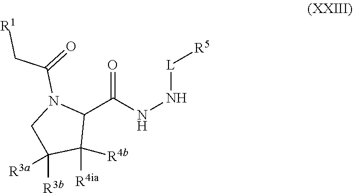

wherein R¹, R^{3a}, R^{3b}, R^{4a}, R^{4b}, R⁵, and L are as defined herein;
with a compound of formula (XI-1),

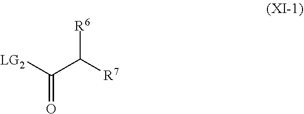

wherein R⁶ and R⁷ are as defined herein and LG₂ is a leaving group;
in the presence of a coupling reagent and a base;
to form a compound of formula (I-b)

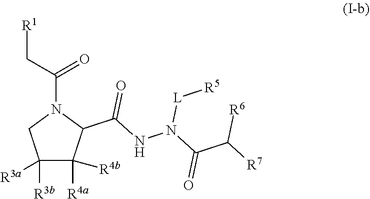

wherein R¹, R^{3a}, R^{3b}, R^{4a}, R^{4b}, R⁵, R⁶, R⁷, and L are as defined herein.

In one embodiment, said leaving group LG₂ is a halogen, in particular chloro.

In one embodiment, the base used in said process is selected from TEA, DIPEA and DMAP.

In one embodiment, the solvent used in said process is DMF or DCM.

In one embodiment, the coupling reagent is selected from T₃P, HATU, PyBOP, HOPO and EDCI/HOBt.

In one aspect, the present invention provides a compound of formula (XI), or a salt thereof,

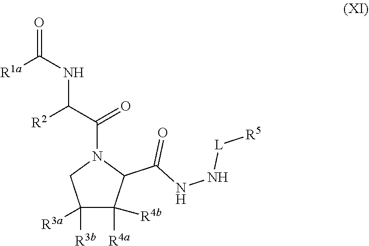

wherein R^{1a}, R², R^{3a}, R^{3b}, R^{4a}, R^{4b}, R⁵, and L are as defined herein.

In one aspect, the present invention provides a compound of formula (XXIII), or a salt thereof,

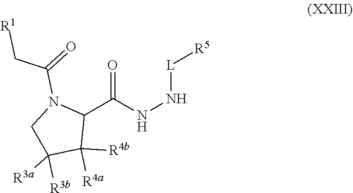

wherein R¹, R^{3a}, R^{3b}, R^{4a}, R^{4b}, R⁵, and L are as defined herein.

### Using the Compounds of the Invention

In one aspect, the present invention provides compounds of formula (I), or pharmaceutically acceptable salts thereof, as described herein for use as therapeutically active substance.

In one aspect, the present invention provides the use of a compound of formula (I) described herein, or a pharmaceutically acceptable salt thereof, for the treatment or prophylaxis of coronavirus infections.

In one aspect, the present invention provides the use of a compound of formula (I) described herein, or a pharmaceutically acceptable salt thereof, for inhibiting the enzymatic activity of 3C-like proteases.

In one aspect, the present invention provides the use of a compound of formula (I) described herein, or a pharmaceutically acceptable salt thereof, for the preparation of a medicament for the treatment or prophylaxis of coronavirus infections.

In one aspect, the present invention provides the use of a compound of formula (I) described herein, or a pharmaceutically acceptable salt thereof, for the preparation of a medicament for inhibiting the enzymatic activity of 3C-like proteases.

In one aspect, the present invention provides a compound of formula (I) described herein, or a pharmaceutically acceptable salt thereof, for use in the treatment or prophylaxis of coronavirus infections.

In one aspect, the present invention provides a compound of formula (I) described herein, or a pharmaceutically acceptable salt thereof, for use in inhibiting the enzymatic activity of 3C-like proteases.

In one aspect, the present invention provides a method of treatment or prophylaxis of coronavirus infections, said method comprising administering a therapeutically active amount of a compound of formula (I) described herein, or a pharmaceutically acceptable salt thereof, to a subject in need.

In one aspect, the present invention provides a method of inhibiting the enzymatic activity of 3C-like proteases, said method comprising contacting a 3C-like protease with a compound of formula (I) described herein, or a pharmaceutically acceptable salt thereof.

In one embodiment, said coronavirus is selected from severe acute respiratory syndrome coronavirus (SARS-CoV), severe acute respiratory syndrome coronavirus 2 (SARS-CoV-2) and Middle East Respiratory Syndrome Coronavirus (MERS-CoV).

In one embodiment, said coronavirus is severe acute respiratory syndrome coronavirus (SARS-CoV).

In one embodiment, said coronavirus is severe acute respiratory syndrome coronavirus 2 (SARS-CoV-2).

In one embodiment, said coronavirus is Middle East Respiratory Syndrome Coronavirus (MERS-CoV).

### Pharmaceutical Compositions and Administration

In one aspect, the present invention provides a pharmaceutical composition comprising a compound of formula (I) as described herein and a therapeutically inert carrier.

In one embodiment, there is provided a pharmaceutical composition according to Example 193 or 194.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### ABBREVIATIONS

Abbreviations used herein are as follows:
aq. Aqueous
ACN acetonitrile
BnBr benzyl bromide
CbzCl benzyl chloroformate
Cbz benzyl formate
CDCl₃ deuterated chloroform
CD₃OD deuterated methanol
DIPEA N, N-diethylpropylamine
DMF dimethyl formamide
DMSO dimethyl sulfoxide
DBU 1,8-Diazabicycloundec-7-ene
EDCI N-Ethyl-N′-(3-dimethylaminopropyl)carbodiimide hydrochloride
EDTA ethylenediaminetetraacetic acid
EtOAc or EA ethyl acetate
FAM carboxyfluorescein
FRET fluorescence resonance energy transfer
HATU (1-[Bis(dimethylamino)methylene]-1H-1,2,3-triazolo[4,5-b]pyridinium 3-oxid hexafluorophosphate)
HEPES 4-(2-hydroxyethyl)-1-piperazineethanesulfonic acid
h hour
HPLC high performance liquid chromatography
HOBt N-hydroxybenzotriazole
HOPO 2-Hydroxypyridine-N-oxide
LiHMDS lithium bis(trimethylsilyl)amide
N mol/L
MS (ESI) mass spectroscopy (electron spray ionization)
min(s) minute(s)
NMR nuclear magnetic resonance
NMP 1-methyl-2-pyrrolidone
obsd. Observed
PE Petroleum ether
prep-HPLC preparative high performance liquid chromatography
PyBOP benzotriazol-1-yl-oxytripyrrolidinophosphonium hexafluorophosphate
RT or rt room temperature
sat. saturated
SFC supercritical fluid chromatography
TAMRA carboxytetramethylrhodamine
TCEP tris(2-carboxyethyl)phosphine
TCFH N,N,N′,N′-tetramethylchloroformamidinium hexafluorophosphate
TEA triethylamine
TFA trifluoroacetic acid
TFAA trifluoroacetic anhydride
THF tetrahydrofuran
T₃P propylphosphonic anhydride

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Alternatively, intermediates and final compounds were purified by preparative HPLC on reversed phase column using X Bridge™ Perp C₁₈ (5 μm, OBD™ 30×100 mm) column, X Bridge™ Perp C₁₈ (20-40 m, OBD™ 30×100 mm) column, Welch Ultimate X Bridge™ SiOH 250*50*10 um column, SunFire™ Perp C₁₈ (5 μm, OBD™ 30×100 mm) column, Phenomenex Luna C18 75*30 mm*3 μm column or Phenomenex Synergi C18 150*25 mm*10 μm column.

For SFC chiral separation, intermediates were separated by chiral column (Daicel chiralpak IC, 5 μm, 30×250 mm), (Daicel chiralpak IC, 10 μm, 30×250 mm), AS (10 μm, 30×250 mm), AD (10 μm, 30×250 mm), Chiralpak IG-3 (50×4.6 mm I.D., 3 μm), using Mettler Toledo Multigram III system SFC, ACSWH-PREP-SFC-C, Waters 80Q preparative SFC or Thar 80 preparative SFC, solvent system: CO₂ and IPA (0.5% TEA in IPA) or CO₂ and MeOH (0.1% NH₃·H₂O in MeOH) or CO₂ and Neu-IPA, back pressure 100 bar, detection UV@254 or 220 nm.

LC/MS spectra were obtained using a Waters UPLC-SQD Mass or SHIMADZU LCMS-2020. Standard LC/MS conditions were as follows (running time 3 mins):

Acidic condition: A: 0.1% formic acid and 1% acetonitrile in H₂O; B: 0.1% formic acid in acetonitrile;

Basic condition: A: 0.05% NH₃·H₂O in H₂O; B: acetonitrile.

Mass spectra (MS): generally only ions which indicate the parent mass are reported, and unless otherwise stated the mass ion quoted is the positive mass ion (M+H)+.

NMR Spectra were obtained by using Bruker Avance 400 MHz.

All reactions involving air-sensitive reagents were performed under an argon atmosphere. Reagents from commercial suppliers were used without further purification unless otherwise noted.

### PREPARATIVE EXAMPLES

### Preparation of Intermediate

### Intermediate 1

### Benzyl (1R,2S,5S)-3-[(2S)-2-amino-3,3-dimethyl-butanoyl]-6,6-dimethyl-3-azabicyclo[3.1.0]hexane-2-carboxylate

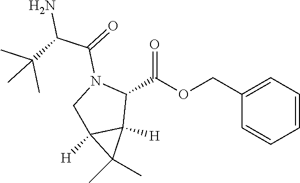 [SMILES: CC(C)(C)[C@H](N)C(=O)N1C[C@H]2[C@@H]([C@H]1C(=O)OCc1ccccc1)C2(C)C]

### Step 1: Preparation of (1R,2S,5S)-3-tert-butoxycarbonyl-6,6-dimethyl-3-azabicyclo[3.1.0]hexane-2-carboxylic acid

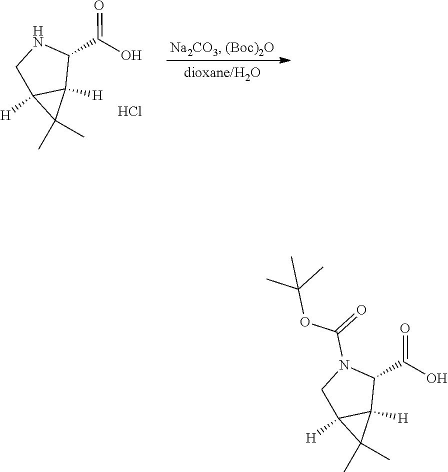 [SMILES: CC1(C)[C@@H]2[C@@H](C(=O)O)NC[C@@H]21 ; Cl ; CC(C)(C)OC(=O)N1C[C@H]2[C@@H]([C@H]1C(=O)O)C2(C)C]

To a solution of Na₂CO₃ (1.37 g, 12.89 mmol) and (1R,2S,5S)-6,6-dimethyl-3-azabicyclo[3.1.0]hexane-2-carboxylic acid; hydrochloride (1.0 g, 6.44 mmol) (Pharmablock, CAS number: 1373205-30-1) in water (15 mL) and 1,4-dioxane (15 mL) was added di-tert-butyldicarbonate (2.1 g, 9.67 mmol). The reaction was stirred at 20° C. for 12 h. The reaction mixture was diluted with H₂O (80 mL) and washed with EtOAc (50 mL). The aqueous layer was acidified with 1M HCl to pH=2-3. The mixture was extracted with EtOAc (50 mL×2). The organic layers were washed with brine (40 mL), dried over Na₂SO₄, filtered and the filtrate was concentrated in vacuum to afford (1R,2S,5S)-3-tert-butoxycarbonyl-6,6-dimethyl-3-azabicyclo[3.1.0]hexane-2-carboxylic acid (1.4 g) as colorless oil.

¹H NMR (400 MHz, CDCl₃) δ: ppm: 4.21 (s, 0.5H), 4.12 (s, 0.5H), 3.69-3.56 (m, 1H), 3.48-3.04 (m, 1H), 1.69 (d, J=7.2 Hz, 0.5H), 1.48-1.39 (m, 10.5H), 1.06-1.05 (m, 3H), 0.99 (d, J=16 Hz, 3H).

### Step 2: Preparation of O2-benzyl O3-tert-butyl (1R,2S,5S)-6,6-dimethyl-3-azabicyclo[3.1.0]hexane-2,3-dicarboxylate

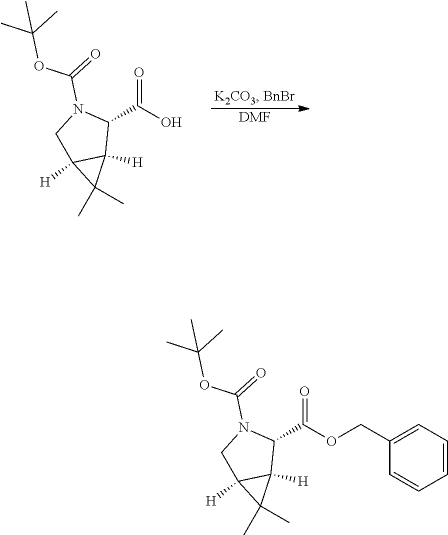

To a solution of (1R,2S,5S)-3-tert-butoxycarbonyl-6,6-dimethyl-3-azabicyclo[3.1.0]hexane-2-carboxylic acid (1.4 g, 5.48 mmol) in DMF (30 mL) was added K₂CO₃ (1.51 g, 10.97 mmol) and BnBr (1.22 g, 7.13 mmol). The reaction mixture was stirred at 25° C. for 2 h. The mixture was diluted with EtOAc (130 mL), washed with water (50 mL) and brine (50 mL×3). The organic layer was dried over Na₂SO₄, filtered and the filtrate was concentrated in vacuum. The residue was purified by silica gel column eluted with PE to PE/EtOAc=4/1 to afford O2-benzyl O3-tert-butyl (1R,2S,5S)-6,6-dimethyl-3-azabicyclo[3.1.0]hexane-2,3-dicarboxylate (1.6 g) as colorless oil.

MS obsd. (ESI⁺) [M+Na]⁺: 368.2

### Step 3: Preparation of benzyl (1R,2S,5S)-6,6-dimethyl-3-azabicyclo[3.1.0]hexane-2-carboxylate

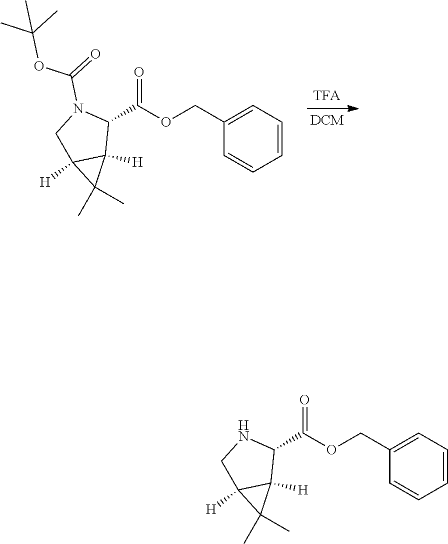 [SMILES: CC(C)(C)OC(=O)N1C[C@H]2[C@@H]([C@H]1C(=O)OCc1ccccc1)C2(C)C ; CC1(C)[C@@H]2[C@@H](C(=O)OCc3ccccc3)NC[C@@H]21]

To a solution of O2-benzyl O3-tert-butyl (1R,2S,5S)-6,6-dimethyl-3-azabicyclo[3.1.0]hexane-2,3-dicarboxylate (1.1 g, 3.18 mmol) in DCM (10 mL) was added TFA (10.0 mL). The reaction mixture was stirred at 25° C. for 1 h. The mixture was concentrated in vacuum to afford (1R,2S,5S)-benzyl 6,6-dimethyl-3-azabicyclo[3.1.0]hexane-2-carboxylate (780 mg) as colorless oil.

MS obsd. (ESI⁺) [M+H]⁺: 246.1

### Step 4: Preparation of benzyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate

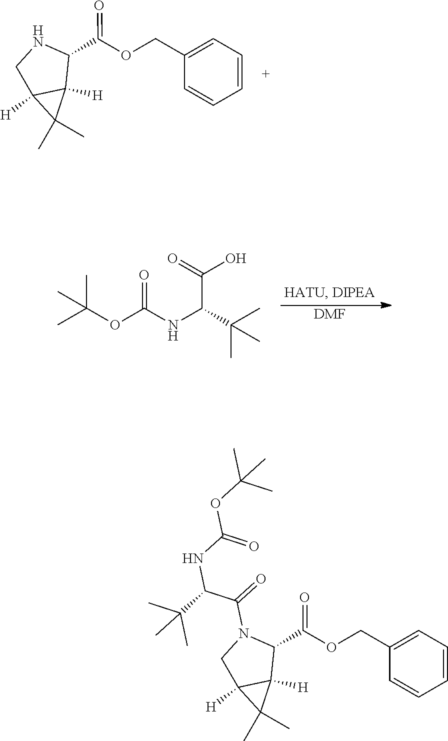

To a solution of (2S)-2-(tert-butoxycarbonylamino)-3,3-dimethyl-butanoic acid (0.74 g, 3.18 mmol) (BePharm, CAS number: 62965-35-9) in DMF (30 mL) was added HATU (1.45 g, 3.82 mmol), DIPEA (2.46 g, 19.08 mmol) and (1R,2S,5S)-benzyl 6,6-dimethyl-3-azabicyclo[3.1.0]hexane-2-carboxylate (0.78 g, 3.18 mmol). The reaction mixture was stirred at 25° C. for 2 h. The mixture was diluted with EtOAc (100 mL), washed with water (50 mL) and brine (50 mL×3). The organic layer was dried over Na₂SO₄, filtered and the filtrate was concentrated in vacuum. The residue was purified by silica gel column eluted with PE/EtOAc=10/1 to afford benzyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate (1.25 g) as colorless oil.

MS obsd. (ESI⁺) [M+H]⁺: 459.4

### Step 5: Preparation of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1)

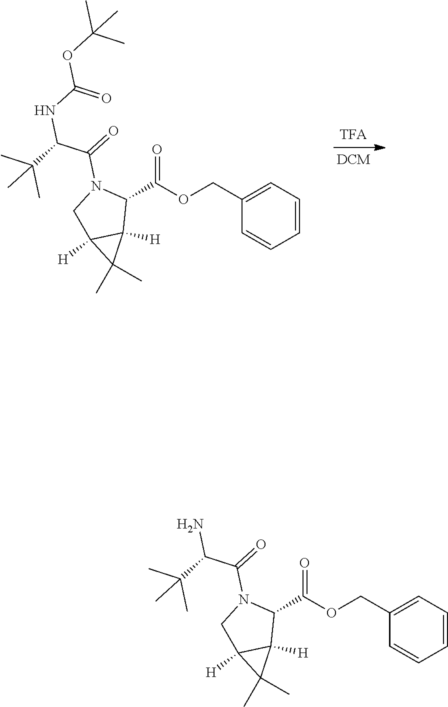 [SMILES: CC(C)(C)OC(=O)N[C@H](C(=O)N1C[C@H]2[C@@H]([C@H]1C(=O)OCc1ccccc1)C2(C)C)C(C)(C)C ; CC(C)(C)[C@H](N)C(=O)N1C[C@H]2[C@@H]([C@H]1C(=O)OCc1ccccc1)C2(C)C]

To a solution of benzyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate (1.25 g, 2.73 mmol) in DCM (10 mL) was added TFA (10 mL). The reaction mixture was stirred at 25° C. for 1 h. The mixture was concentrated in vacuum to afford benzyl (1R,2S,5S)-3-[(2S)-2-amino-3,3-dimethyl-butanoyl]-6,6-dimethyl-3-azabicyclo[3.1.0]hexane-2-carboxylate (0.97 g, Intermediate 1) as colorless oil.

MS obsd. (ESI⁺) [M+H]⁺: 359.3.

### Intermediate 2

### Benzyl (1R,2S,5S)-3-[(2S)-2-amino-2-cyclopropyl-acetyl]-6,6-dimethyl-3-azabicyclo[3.1.0]hexane-2-carboxylate

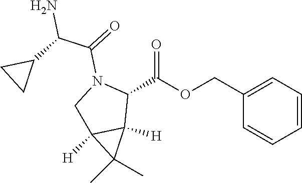 [SMILES: CC1(C)[C@@H]2[C@@H](C(=O)OCc3ccccc3)N(C(=O)[C@@H](N)C3CC3)C[C@@H]21]

The title compound was prepared in analogy to the procedure described for the preparation of Intermediate 1, by (2S)-2-(tert-butoxycarbonylamino)-2-cyclopropyl-acetic acid (BePharm, CAS number: 155976-13-9) instead of (2S)-2-(tert-butoxycarbonylamino)-3,3-dimethyl-butanoic acid to afford benzyl (1R,2S,5S)-3-[(2S)-2-amino-2-cyclopropyl-acetyl]-6,6-dimethyl-3-azabicyclo[3.1.0]hexane-2-carboxylate (Intermediate 2) as yellow oil.

MS obsd. (ESI⁺) [(M+H)⁺]: 343.1.

### Intermediate 3

### Benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate

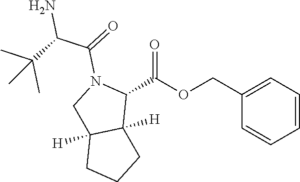 [SMILES: CC(C)(C)[C@H](N)C(=O)N1C[C@@H]2CCC[C@@H]2[C@H]1C(=O)OCc1ccccc1]

The title compound was prepared in analogy to the procedure described for the preparation of Intermediate 1, by using (3S,3aS,6aR)-2-tert-butoxycarbonyl-3,3a,4,5,6,6a-hexahydro-1H-cyclopenta[c]pyrrole-3-carboxylic acid (AstaTech, CAS number: 597569-42-1) instead of (1R,2S,5S)-3-tert-butoxycarbonyl-6,6-dimethyl-3-azabicyclo[3.1.0]hexane-2-carboxylic acid to afford benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate (Intermediate 3) as yellow oil.

MS obsd. (ESI⁺) [(M+H)⁺]: 359.3.

### Intermediate 4

### Benzyl (1S,2S,5R)-3-[(2S)-2-amino-3,3-dimethyl-butanoyl]-3-azabicyclo[3.2.0]heptane-2-carboxylate

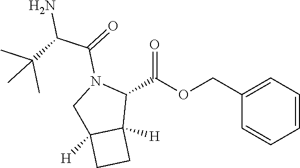 [SMILES: CC(C)(C)[C@H](N)C(=O)N1C[C@@H]2CC[C@@H]2[C@H]1C(=O)OCc1ccccc1]

The title compound was prepared in analogy to the procedure described for the preparation of Intermediate 1, by using (1S,2S,5R)-3-azabicyclo[3.2.0]heptane-2-carboxylic acid (Pharmablock, CAS number: 77882-10-1) instead of (1R,2S,5S)-6,6-dimethyl-3-azabicyclo[3.1.0]hexane-2-carboxylic acid; hydrochloride to afford benzyl (1S,2S,5R)-3-[(2S)-2-amino-3,3-dimethyl-butanoyl]-3-azabicyclo[3.2.0]heptane-2-carboxylate (Intermediate 4) as yellow oil.

MS obsd. (ESI⁺) [(M+H)⁺]: 345.3.

### Intermediate 5

### Benzyl (6S)-5-[(2S)-2-amino-3,3-dimethyl-butanoyl]-5-azaspiro[2.4]heptane-6-carboxylate

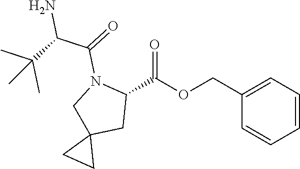 [SMILES: CC(C)(C)[C@H](N)C(=O)N1CC2(CC2)C[C@H]1C(=O)OCc1ccccc1]

The title compound was prepared in analogy to the procedure described for the preparation of Intermediate 1, by using (6S)-5-tert-butoxycarbonyl-5-azaspiro[2.4]heptane-6-carboxylic acid (Wuxiapptec, catalog) instead of (1R,2S,5S)-3-tert-butoxycarbonyl-6,6-dimethyl-3-azabicyclo[3.1.0]hexane-2-carboxylic acid to afford benzyl (6S)-5-[(2S)-2-amino-3,3-dimethyl-butanoyl]-5-azaspiro[2.4]heptane-6-carboxylate (Intermediate 5) as yellow oil.

MS obsd. (ESI⁺) [(M+H)⁺]: 345.2.

### Intermediate 6

### Benzyl (2S,4R)-1-[(2S)-2-amino-3,3-dimethyl-butanoyl]-4-methyl-pyrrolidine-2-carboxylate

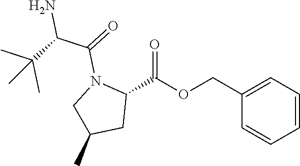 [SMILES: C[C@@H]1C[C@@H](C(=O)OCc2ccccc2)N(C(=O)[C@@H](N)C(C)(C)C)C1]

The title compound was prepared in analogy to the procedure described for the preparation of Intermediate 1, by using (2S,4R)-4-methylpyrrolidine-2-carboxylic acid; hydrochloride (Wuxiapptec, CAS number: 365280-18-8) instead of (1R,2S,5S)-6,6-dimethyl-3-azabicyclo[3.1.0]hexane-2-carboxylic acid; hydrochloride to afford benzyl (2S,4R)-1-[(2S)-2-amino-3,3-dimethyl-butanoyl]-4-methyl-pyrrolidine-2-carboxylate (Intermediate 6) as yellow oil.

MS obsd. (ESI⁺) [(M+H)⁺]: 333.2.

### Intermediate 7

### Benzyl (2S)-1-[(2S)-2-amino-3,3-dimethyl-butanoyl]-4,4-dimethyl-pyrrolidine-2-carboxylate

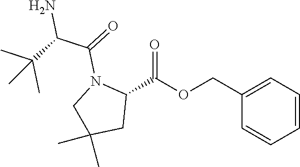 [SMILES: CC1(C)C[C@@H](C(=O)OCc2ccccc2)N(C(=O)[C@@H](N)C(C)(C)C)C1]

The title compound was prepared in analogy to the procedure described for the preparation of Intermediate 1, by using (2S)-4,4-dimethylpyrrolidine-2-carboxylic acid; hydrochloride (Porse Fine Chemical, CAS number: 1443252-76-3) instead of (1R,2S,5S)-6,6-dimethyl-3-azabicyclo[3.1.0]hexane-2-carboxylic acid; hydrochloride to afford benzyl (2S)-1-[(2S)-2-amino-3,3-dimethyl-butanoyl]-4,4-dimethyl-pyrrolidine-2-carboxylate (Intermediate 7) as yellow oil.

MS obsd. (ESI⁺) [(M+H)⁺]: 347.2.

### Intermediate 8

### Benzyl (1S,2S,5R)-3-[(2S)-2-amino-3,3-dimethyl-butanoyl]-3-azabicyclo[3.1.0]hexane-2-carboxylate

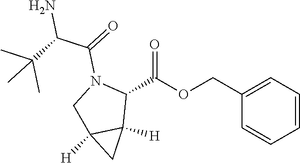 [SMILES: CC(C)(C)[C@H](N)C(=O)N1C[C@@H]2C[C@@H]2[C@H]1C(=O)OCc1ccccc1]

The title compound was prepared in analogy to the procedure described for the preparation of Intermediate 1, by using (1S,2S,5R)-3-tert-butoxycarbonyl-3-azabicyclo[3.1.0]hexane-2-carboxylic acid (Pharmablock, CAS number: 400720-05-0) instead of (1R,2S,5S)-3-tert-butoxycarbonyl-6,6-dimethyl-3-azabicyclo[3.1.0]hexane-2-carboxylic acid to afford benzyl (1S,2S,5R)-3-[(2S)-2-amino-3,3-dimethyl-butanoyl]-3-azabicyclo[3.1.0]hexane-2-carboxylate (Intermediate 8) as yellow oil.

MS obsd. (ESI⁺) [(M+H)⁺]: 331.1.

### Intermediate 9

### Tert-butyl N-amino-N-[[(3S)-2-oxopyrrolidin-3-yl]methyl]carbamate

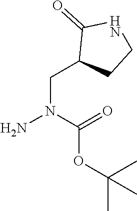 [SMILES: CC(C)(C)OC(=O)N(N)C[C@@H]1CCNC1=O]

### Step 1: Preparation of tert-butyl 3-methylene-2-oxo-pyrrolidine-1-carboxylate

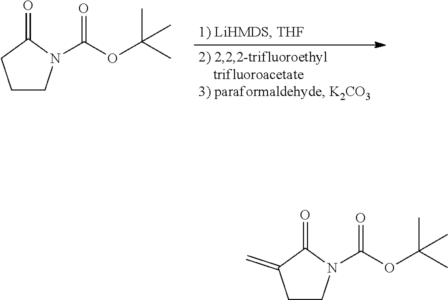

A solution of tert-butyl 2-oxopyrrolidine-1-carboxylate (100.0 g, 539.9 mmol)(Accela, CAS number: 85909-08-6) in THF (1000 mL) was added to a stirring solution of LiHMDS (1133 mL, 1134 mmol) at 0° C. The reaction mixture was allowed to warm up to 20° C. over 30 min before 2,2,2-trifluoroethyl trifluoroacetate (211.69 g, 1080 mmol) was added. Stirring was continued for additional 20 min at 20° C. before the reaction was quenched with saturated NH₄Cl (500 mL). The mixture was extracted with EtOAc (600 mL×2). The combined organic layers were dried over Na₂SO₄, filtered and concentrated in vacuum. The residue was dissolved in toluene (1000 mL) then formaldehyde (48.64 g, 1620 mmol) and K₂CO₃ (164.16 g, 1188 mmol) were added into the solution. The reaction mixture was heated at 110° C. for 2 h. The mixture was diluted with EtOAc (800 mL), washed with brine (200 mL×3), dried over anhydrous Na₂SO₄, filtered and concentrated in vacuum to afford tert-butyl 3-methylene-2-oxo-pyrrolidine-1-carboxylate (80 g) as light yellow oil.

MS obsd. (ESI⁺) [M-Bu+H]⁺: 142.1.

### Step 2: Preparation of tert-butyl 3-[(2-benzyloxycarbonylhydrazino)methyl]-2-oxo-pyrrolidine-1-carboxylate

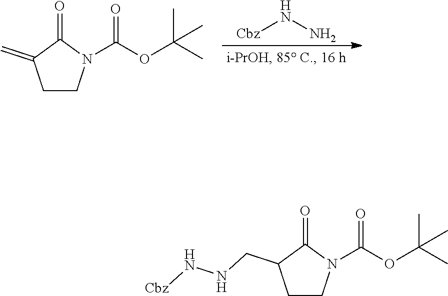 [SMILES: C=C1CCN(C(=O)OC(C)(C)C)C1=O ; NNC(=O)OCc1ccccc1 ; CC(C)(C)OC(=O)N1CCC(CNNC(=O)OCc2ccccc2)C1=O]

To a solution of tert-butyl 3-methylene-2-oxo-pyrrolidine-1-carboxylate (80.0 g, 405.62 mmol) in i-PrOH (800 mL) was added benzyl N-aminocarbamate (67.41 g, 405.62 mmol). The reaction mixture was stirred at 85° C. for 12 h under N₂. The mixture was concentrated in vacuum to afford the crude product tert-butyl 3-[(2-benzyloxycarbonylhydrazino)methyl]-2-oxo-pyrrolidine-1-carboxylate (147 g) as yellow oil.

MS obsd. (ESI⁺) [2M+H]⁺: 727.2.

### Step 3: Preparation of benzyl N-[(2-oxopyrrolidin-3-yl)methylamino]carbamate

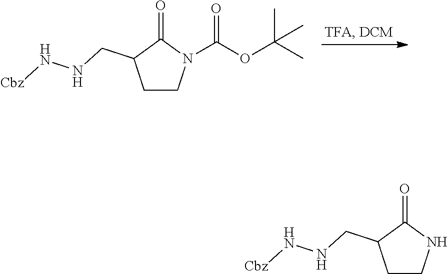 [SMILES: CC(C)(C)OC(=O)N1CCC(CNNC(=O)OCc2ccccc2)C1=O ; O=C(NNCC1CCNC1=O)OCc1ccccc1]

To a solution of tert-butyl 3-[(2-benzyloxycarbonylhydrazino)methyl]-2-oxo-pyrrolidine-1-carboxylate (147.0 g, 404.5 mmol) in DCM (700 mL) was added TFA (500 mL). The mixture was stirred at 20° C. for 2 h. The reaction mixture was concentrated in vacuum to afford benzyl N-[(2-oxopyrrolidin-3-yl)methylamino]carbamate (147.0 g) as yellow oil.

MS obsd. (ESI⁺) [M+H]⁺: 264.0.

### Step 4: Preparation of tert-butyl N-(benzyloxycarbonylamino)-N-[(2-oxopyrrolidin-3-yl)methyl]carbamate

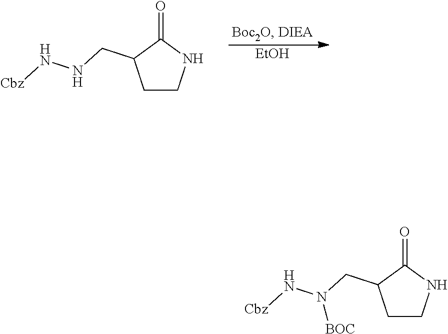 [SMILES: "EtOH" ; O=C(NNCC1CCNC1=O)OCc1ccccc1 ; CC(C)(C)OC(=O)N(CC1CCNC1=O)NC(=O)OCc1ccccc1]

To a solution of benzyl N-[(2-oxopyrrolidin-3-yl)methylamino]carbamate (150.0 g, 569.71 mmol) in EtOH (800 mL) was added DIPEA (220.9 g, 1709 mmol) and Boc₂O (149.2 g, 683.66 mmol). The reaction mixture was stirred at 50° C. for 12 h. The mixture was concentrated in vacuum. The residue was diluted with EtOAc (1000 mL), washed with brine (200 mL), dried over anhydrous Na₂SO₄, filtered and concentrated to give the crude product. The crude product was purified by silica gel column eluted with PE/EtOAc=1/1 to EtOAc to afford an impure product which was conducted a further purification by reverse flash (0.1% TFA) to afford tert-butyl N-(benzyloxycarbonylamino)-N-[(2-oxopyrrolidin-3-yl)methyl]carbamate (50.0 g) as light yellow oil.

MS obsd. (ESI⁺) [M-Boc+H]⁺: 264.2.

### Step 5: Preparation of tert-butyl N-amino-N-[[(3S)-2-oxopyrrolidin-3-yl]methyl]carbamate

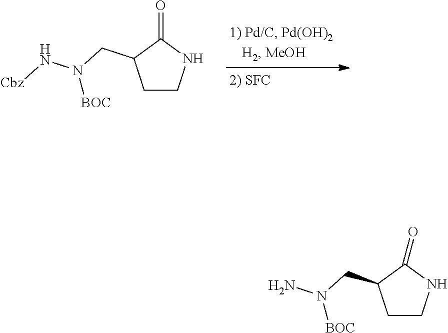 [SMILES: CC(C)(C)OC(=O)N(CC1CCNC1=O)NC(=O)OCc1ccccc1 ; CC(C)(C)OC(=O)N(N)C[C@@H]1CCNC1=O]

To a solution of tert-butyl N-(benzyloxycarbonylamino)-N-[(2-oxopyrrolidin-3-yl)methyl]carbamate (160.0 g, 440.27 mmol) in methanol (1500 mL) was added Pd/C (15.0 g) and Pd(OH)₂/C (15.0 g) under N₂. The reaction mixture was degassed under vacuum and purged H₂ for 3 times. The resulting mixture was stirred at 25° C. for 12 h under H₂ balloon. The mixture was filtered through a pad of celite and the filtrate was concentrated in vacuum to afford tert-butyl N-amino-N-[[(3S)-2-oxopyrrolidin-3-yl]methyl]carbamate (100 g) as colorless oil. The racemate was split by SFC preparation (Column: Phenomenex-Cellulose-2 (250 mm*50 mm, 10 um); Condition: phase A for CO₂, phase B for Neu-MeOH: B %: 45-45%; Flow Rate (mL/min): 220; Gradient time: 3.55 min, injections: 440) to afford tert-butyl N-amino-N-[[(3S)-2-oxopyrrolidin-3-yl]methyl]carbamate (41.4 g, Intermediate 9) as a light yellow solid.

¹H NMR (400 MHz, CDCl₃) δ: ppm 5.77 (s, 1H), 3.75-3.55 (m, 2H), 3.45-3.30 (m, 2H), 2.85-2.75 (m, 1H), 2.30-2.20 (m, 1H), 1.99-1.89 (m, 1H), 1.51-1.42 (m, 9H)

MS obsd. (ESI⁺) [M+H]⁺: 230.1

SFC: Retention time=1.615 min, ee %=98.05%

### Intermediate 10

### (2R)-2-chloro-2-fluoro-acetyl chloride

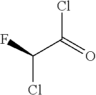

### Step 1: Preparation of 2-chloro-2-fluoroacetic acid

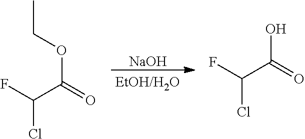

To a solution of ethyl chlorofluoroacetate (1000 g, 7142 mmol) (Aldrich, CAS number: 401-56-9) in ethanol (9 L) was added water (1 L) and NaOH (313 g, 7826 mmol). The reaction mixture was stirred at 25° C. for 12 h. ¹H NMR showed the reaction was complete. The mixture was concentrated in vacuum to remove most of EtOH. Then the residue was diluted with water (1500 mL) and acidified with 2M HCl to pH=4-5. The mixture was extracted with MTBE (1 L×4). The combined organic layers were dried over Na₂SO₄. The mixture was filtered and concentrated to give the crude product 2-chloro-2-fluoroacetic acid (718 g) as colorless oil.

¹H NMR (400 MHz, CDCl₃) δ: ppm 6.16 (d, J=50.4 Hz, 1H).

### Step 2: Preparation of (2R)-2-chloro-2-fluoro-acetic acid

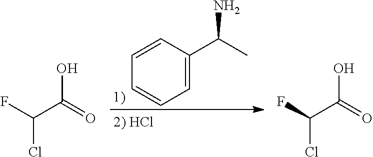

To a solution of 2-chloro-2-fluoro-acetic acid (718.0 g, 3191 mmol) in EtOAc (3000 mL) was added a solution of (S)-1-phenylethanamine (386.7 g, 3191 mmol) in EtOAc (3000 mL) at 0° C. The mixture was stirred at 0° C. for 2 h and then stood overnight. The reaction mixture was filtered and the filter cake was dissolved in acetone (760 g in 7600 mL) at 80° C. The resulting solution was slowly cooled to 20° C. and stood overnight. The precipitate was filtrated, collected and dissolved in acetone (100 g/L) at 80° C. (The recrystallization operation was repeated for 2 times). The collected solid was triturated in acetone for 3 times (acetone, 100 g/500 mL). The solid was collected and dissolved in water (1 L) and acidified with 1M HCl (750 mL). The mixture was extracted with MTBE (1 L×3). The combined organic layers were dried over Na₂SO₄, filtered and concentrated in vacuum to afford (2R)-2-chloro-2-fluoro-acetic acid (121.15 g, 61% purity, containing MTBE) as yellow oil.

¹H NMR (400 MHz, CDCl₃) δ: ppm 6.29 (d, J=50.8 Hz, 1H).

### Step 3: Preparation of (2R)-2-chloro-2-fluoro-acetyl chloride

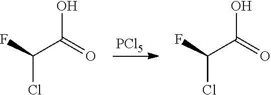

A mixture of (2R)-2-chloro-2-fluoro-acetic acid (12.0 g, 106.68 mmol) and PCl₅ (24.5 g, 117.47 mmol) was stirred at 20° C. for 1 h and at 70° C. for 1 h. ¹H NMR showed the reaction was complete. Then (2R)-2-chloro-2-fluoro-acetyl chloride (22.8 g, 40% purity with POCl₃ and MTBE, Intermediate 10) was collected by distillation (70° C., 20 mmHg) as a colorless liquid.

¹H NMR (400 MHz, CDCl₃) δ: ppm 6.40 (d, J=50.8 Hz, 1H).

### Intermediate 11

### tert-Butyl N-amino-N-(1H-pyrazol-3-ylmethyl)carbamate

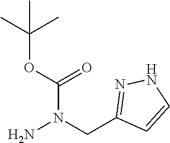

### Step 1: Preparation of benzyl N-(1H-pyrazol-3-ylmethylamino)carbamate

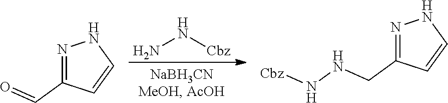

To a solution of 1H-pyrazole-3-carbaldehyde (5.0 g, 52.03 mmol) (BePharm, CAS number: 3920-50-1) and benzyl carbazate (8.68 g, 52.20 mmol) in methanol (100 mL) was added HOAc (10.0 mL, 174.85 mmol). The mixture was stirred at 25° C. for 3 h. Then to the mixture was added NaBH₃CN (19.6 g, 312.21 mmol). The mixture was stirred at 60° C. for 12 h. The reaction was concentrated in vacuum to remove methanol. The residue was diluted with H₂O (50 mL) and extracted with EtOAc (50 mL×3). The organic layers were dried over anhydrous Na₂SO₄, filtered and concentrated. The residue was purified by reverse column (condition: 330 g Flash Column Welch Ultimate XB_C₁₈ 20-40 um; 120 A, ACN in water (0.1% TFA), 26%˜26%, 100 mL/min). The fraction was basified with saturated aqueous NaHCO₃ to pH=9 and extracted with EtOAc (100 mL×3). The organic phase was dried over anhydrous Na₂SO₄, filtered and concentrated in vacuum to afford benzyl N-(1H-pyrazol-3-ylmethylamino)carbamate (10 g) as yellow oil.

MS obsd. (ESI⁺) [(M+H)⁺]: 246.9

### Step 2: Preparation of tert-butyl N-(benzyloxycarbonylamino)-N-(1H-pyrazol-3-ylmethyl)carbamate

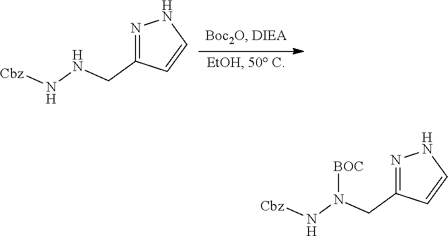

To a solution of benzyl N-(1H-pyrazol-3-ylmethylamino)carbamate (3.0 g, 12.18 mmol) in EtOH (40 mL) was added DIPEA (6.29 g, 48.67 mmol) and Boc₂O (5.32 g, 24.38 mmol). The mixture was stirred at 50° C. for 12 h. The reaction mixture was concentrated in vacuum. The residue was purified by silica gel column eluted with EtOAc/PE=1/1 to EtOAc to afford tert-butyl N-(benzyloxycarbonylamino)-N-(1H-pyrazol-3-ylmethyl)carbamate (1.2 g) as colorless oil.

MS obsd. (ESI⁺) [(M+H)⁺]: 347.0.

### Step 3: Preparation of tert-butyl N-amino-N-(1H-pyrazol-3-ylmethyl)carbamate

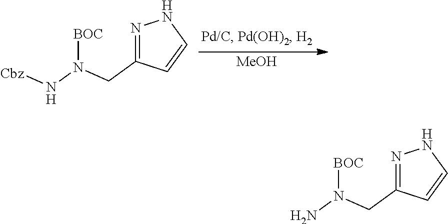 [SMILES: "MeOH" ; CC(C)(C)OC(=O)N(Cc1cc[nH]n1)NC(=O)OCc1ccccc1 ; CC(C)(C)OC(=O)N(N)Cc1cc[nH]n1]

To a solution of tert-butyl N-(benzyloxycarbonylamino)-N-(1H-pyrazol-3-ylmethyl)carbamate (1.2 g, 3.46 mmol) in MeOH (15 mL) was added Pd/C (120.0 mg) and Pd(OH)₂ (120.0 mg). The mixture was degassed in vacuum and purged H₂ for 3 times. The resulting mixture was stirred at 25° C. for 2 h under hydrogen balloon (760 mmHg). The reaction mixture was filtered through a celite pad and the filtrate was concentrated in vacuum. The residue was purified by silica gel column eluted with EtOAc to MeOH/EtOAc=10/1 to afford tert-butyl N-amino-N-(1H-pyrazol-3-ylmethyl)carbamate (600 mg, Intermediate 11) as colorless oil.

¹H NMR (400 MHz, CD₃OD) δ: ppm 7.53 (d, J=2.0 Hz, 1H), 6.24 (d, J=1.6 Hz, 1H), 4.61 (s, 2H), 1.49 (s, 9H).

MS obsd. (ESI⁺) [(2M+Na)⁺]: 447.3.

### Intermediate 12

### 5-(Trifluoromethyl)isoxazole-3-carboxylic acid

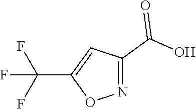 [SMILES: O=C(O)c1cc(C(F)(F)F)on1]

### Step 1: Preparation of ethyl 5-(trifluoromethyl)isoxazole-3-carboxylate

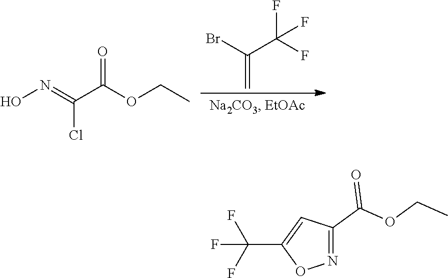

To a solution of ethyl (Z)-2-chloro-2-(hydroxyimino)acetate (3.0 g, 19.8 mmol) (Accela, CAS number: 14337-43-0) in EtOAc (50 mL) was added 2-bromo-3,3,3-trifluoroprop-1-ene (10.4 g, 59.39 mmol) and NaHCO₃ (5.5 g, 65.32 mmol). The mixture was stirred at 20° C. for 12 h. The reaction mixture was filtered and the filtrate was concentrated in vacuum at 30° C. to afford ethyl 5-(trifluoromethyl)isoxazole-3-carboxylate (3.75 g) as yellow oil.

¹H NMR (400 MHz, DMSO-d₆) δ: ppm 7.93 (s, 1H), 4.44-4.39 (m, 2H), 1.34 (t, J=7.2 Hz, 3H).

### Step 2: Preparation of 5-(trifluoromethyl)isoxazole-3-carboxylic acid

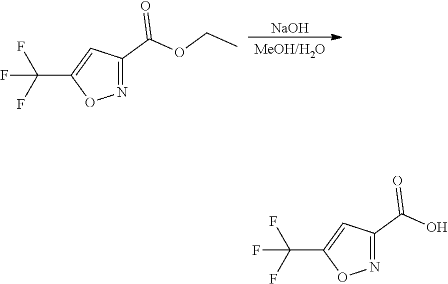

To a solution of ethyl 5-(trifluoromethyl)isoxazole-3-carboxylate (3.75 g, 17.93 mmol) in methanol (40 mL) was added the solution of NaOH (1.58 g, 39.45 mmol) in water (40 mL) at 0° C. The mixture was stirred at 20° C. for 1 h. The reaction mixture was concentrated in vacuum to remove MeOH. The residue was diluted with EtOAc (80 mL) and washed with 1M HCl (40 mL). The aqueous phase was extracted with EtOAc (80 mL×6). The organic phase was dried over Na₂SO₄, filtered and the filtrate was concentrated. The residue was purified by silica gel column eluted with PE/EtOAc=7/3 to 1/1 to afford 5-(trifluoromethyl)isoxazole-3-carboxylic acid (2.3 g, Intermediate 12) as a white solid.

¹H NMR (400 MHz, CDCl₃) δ: ppm 7.39 (br. s, 1H), 7.18 (s, 1H).

### Intermediate 13

### 2-(3,5-Difluorophenoxy)acetyl chloride

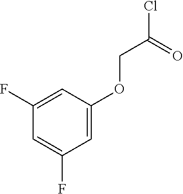 [SMILES: O=C(Cl)COc1cc(F)cc(F)c1]

### Step 1: Preparation of ethyl 2-(3,5-difluorophenoxy)acetate

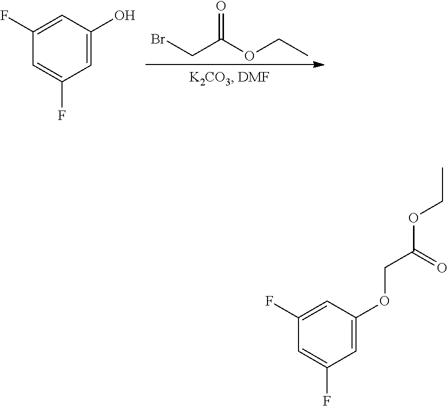

To a solution of 3,5-difluorophenol (475 mg, 3.65 mmol) in DMF (10 mL) was added K₂CO₃ (1008 mg, 7.3 mmol) and ethyl bromoacetate (0.4 mL, 3.65 mmol). The mixture was stirred at 25° C. for 4 h. The mixture was diluted with EtOAc (50 mL) and poured into water (50 mL). The aqueous phase was extracted with EtOAc (30 mL×3). The combined organic layers were washed with brine (30 mL) and dried over Na₂SO₄. After filtration and concentration, the residue was purified by silica gel column eluted with EtOAc/PE=50/1 to 10/1 to afford ethyl 2-(3,5-difluorophenoxy)acetate (650 mg) as colorless liquid.

### Step 2: Preparation of 2-(3,5-difluorophenoxy)acetic acid

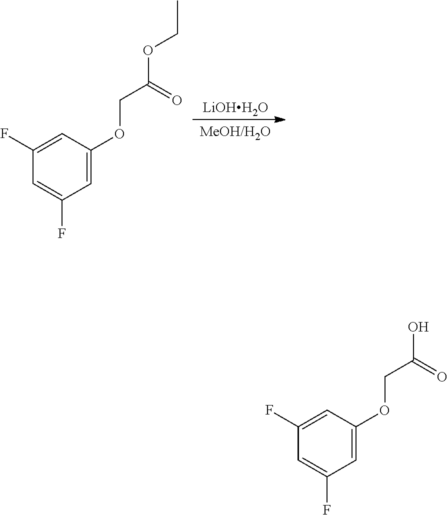

To a solution of ethyl 2-(3,5-difluorophenoxy)acetate (150 mg, 0.690 mmol) in MeOH (5 mL) and water (2 mL) was added LiOH·H₂O (73 mg, 1.73 mmol). The mixture was stirred at 20° C. for 1 h. The mixture was diluted with H₂O (30 mL) and washed with EtOAc (20 mL×2). The aqueous phase was acidified with conc. HCl to pH=5-6 and extracted with EtOAc (30 mL×3). The combined organic layers were dried over Na₂SO₄, filtered and concentrated in vacuum to afford 2-(3,5-difluorophenoxy)acetic acid (130 mg) as a white solid.

### Step 3: Preparation of 2-(3,5-difluorophenoxy)acetyl chloride

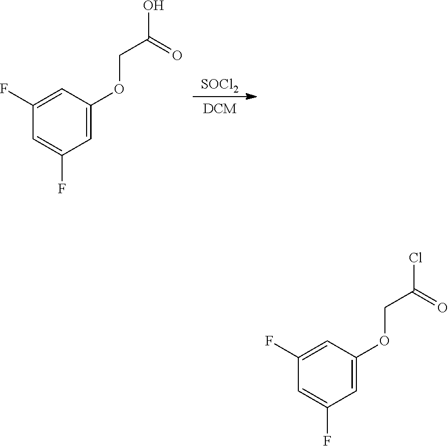

To a solution of 2-(3,5-difluorophenoxy)acetic acid (110 mg, 0.580 mmol) in DCM (5 mL) was added SOCl₂ (1 mL). The mixture was stirred at 40° C. for 2 h. The mixture was concentrated in vacuum to afford 2-(3,5-difluorophenoxy)acetyl chloride (120 mg, Intermediate 13) as colorless oil.

### Intermediate 14

### tert-butyl N-[[(1R,2S,5S)-6,6-dimethyl-3-azabicyclo[3.1.0]hexane-2-carbonyl]amino]-N-[[(3S)-2-oxopyrrolidin-3-yl]methyl]carbamate

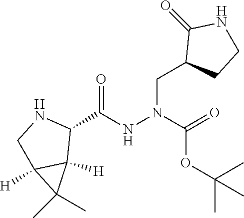 [SMILES: CC(C)(C)OC(=O)N(C[C@@H]1CCNC1=O)NC(=O)[C@H]1NC[C@H]2[C@@H]1C2(C)C]

### Step 1: Preparation of (1R,2S,5S)-3-benzyloxycarbonyl-6,6-dimethyl-3-azabicyclo[3.1.0]hexane-2-carboxylic acid

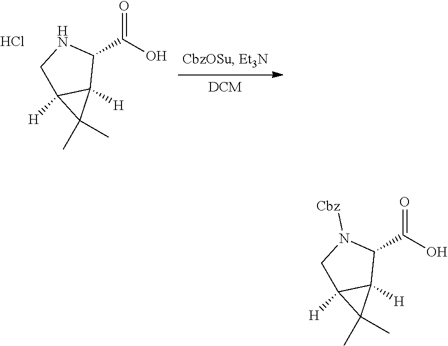 [SMILES: Cl ; CC1(C)[C@@H]2[C@@H](C(=O)O)NC[C@@H]21 ; CC1(C)[C@@H]2[C@@H](C(=O)O)N(C(=O)OCc3ccccc3)C[C@@H]21]

To a solution of (1R,2S,5S)-6,6-dimethyl-3-azabicyclo[3.1.0]hexane-2-carboxylic acid hydrochloride (1.5 g, 7.83 mmol) in DCM (20 mL) was added TEA (2.37 g, 23.48 mmol) and CbzOsu (2.93 g, 11.74 mmol) at 0° C. The mixture was warmed up to 25° C. and stirred at 25° C. for 16 h. The mixture was concentrated in vacuum. The residue was purified by reverse flash chromatography eluted with ACN in H₂O (0.1% TFA)=0˜64% to afford (1R,2S,5S)-3-benzyloxycarbonyl-6,6-dimethyl-3-azabicyclo[3.1.0]hexane-2-carboxylic acid (1.96 g) as black oil.

¹H NMR (400 MHz, CD₃OD) δ: ppm 7.37-7.27 (m, 5H), 5.55-5.75 (br. s, 1H), 5.17-5.11 (m, 2H), 4.29 (d, J=12.4 Hz, 1H), 3.74-3.65 (m, 1H), 3.55 (t, J=12.8 Hz, 1H), 1.67-1.53 (m, 1H), 1.50-1.43 (m, 1H), 1.07 (d, J=2.4 Hz, 3H), 0.98 (d, J=5.6 Hz, 1H).

MS obsd. (ESI⁺) [(M+Na)⁺]: 311.9

### Step 2: Preparation of benzyl (1R,2S,5S)-2-[[tert-butoxycarbonyl-[[(3S)-2-oxopyrrolidin-3-yl]methyl]amino]carbamoyl]-6,6-dimethyl-3-azabicyclo[3.1.0]hexane-3-carboxylate

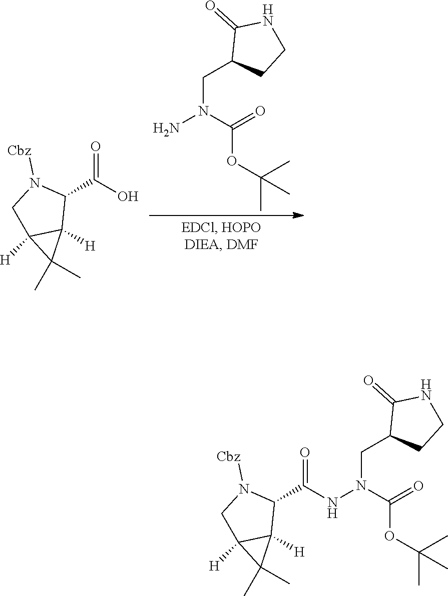 [SMILES: CC1(C)[C@@H]2[C@@H](C(=O)O)N(C(=O)OCc3ccccc3)C[C@@H]21 ; CC(C)(C)OC(=O)N(N)C[C@@H]1CCNC1=O ; CC(C)(C)OC(=O)N(C[C@@H]1CCNC1=O)NC(=O)[C@@H]1[C@@H]2[C@H](CN1C(=O)OCc1ccccc1)C2(C)C]

To a solution of (1R,2S,5S)-3-benzyloxycarbonyl-6,6-dimethyl-3-azabicyclo[3.1.0]hexane-2-carboxylic acid (1.4 g, 4.84 mmol) in DMF (20 mL) was added DIPEA (3.1 g, 24.19 mmol), EDCI (1113 mg, 5.81 mmol) and HOPO (645 mg, 5.81 mmol) at 0° C. Then tert-butyl N-amino-N-[[(3S)-2-oxopyrrolidin-3-yl]methyl]carbamate (1.1 g, 4.84 mmol) was added to the reaction mixture. The resulting mixture was warmed up to 25° C. and stirred for 16 h. The mixture was purified by reverse flash chromatography eluted with ACN in H₂O (0.1% TFA)=0˜54% to afford benzyl (1R,2S,5S)-2-[[tert-butoxycarbonyl-[[(3S)-2-oxopyrrolidin-3-yl]methyl]amino]carbamoyl]-6,6-dimethyl-3-azabicyclo[3.1.0]hexane-3-carboxylate (1.9 g) as yellow oil.

MS obsd. (ESI⁺) [(M+H)⁺]: 501.3.

### Step 3: Preparation of tert-butyl N-[[(1R,2S,5S)-6,6-dimethyl-3-azabicyclo[3.1.0]hexane-2-carbonyl]amino]-N-[[(3S)-2-oxopyrrolidin-3-yl]methyl]carbamate

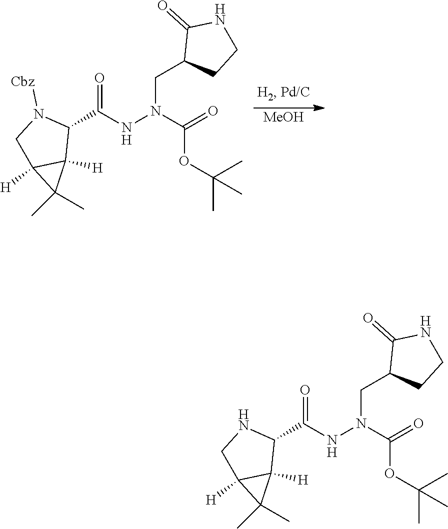 [SMILES: "MeOH" ; CC(C)(C)OC(=O)N(C[C@@H]1CCNC1=O)NC(=O)[C@@H]1[C@@H]2[C@H](CN1C(=O)OCc1ccccc1)C2(C)C ; CC(C)(C)OC(=O)N(C[C@@H]1CCNC1=O)NC(=O)[C@H]1NC[C@H]2[C@@H]1C2(C)C]

To a solution of benzyl (1R,2S,5S)-2-[[tert-butoxycarbonyl-[[(3S)-2-oxopyrrolidin-3-yl]methyl]amino]carbamoyl]-6,6-dimethyl-3-azabicyclo[3.1.0]hexane-3-carboxylate (2.53 g, 5.04 mmol) in MeOH (30 mL) was added Pd/C (100 mg, 10% purity) under N₂ atmosphere. The suspension was degassed and purged with H₂ for 3 times. The mixture was stirred under a H₂ balloon at 25° C. for 2 h. The mixture was filtered and the filtrate was concentrated to afford tert-butyl N-[[(1R,2S,5S)-6,6-dimethyl-3-azabicyclo[3.1.0]hexane-2-carbonyl]amino]-N-[[(3S)-2-oxopyrrolidin-3-yl]methyl]carbamate (1.6 g, Intermediate 14) as yellow oil.

MS obsd. (ESI⁺) [(M+H)⁺]: 367.2.

### Intermediate 15

### Benzyl (2S,4R)-1-[(2S)-2-amino-3,3-dimethyl-butanoyl]-4-(trifluoromethyl)pyrrolidine-2-carboxylate

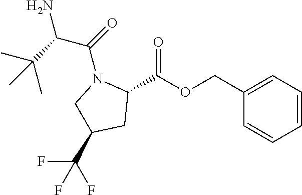 [SMILES: CC(C)(C)[C@H](N)C(=O)N1C[C@H](C(F)(F)F)C[C@H]1C(=O)OCc1ccccc1]

The title compound was prepared in analogy to the procedure described for the preparation of Intermediate 1, by using (2S,4R)-1-tert-butoxycarbonyl-4-(trifluoromethyl)pyrrolidine-2-carboxylic acid (PharmaBlock, CAS number: 470482-44-1) instead of (1R,2S,5S)-3-tert-butoxycarbonyl-6,6-dimethyl-3-azabicyclo[3.1.0]hexane-2-carboxylic acid to afford benzyl (2S,4R)-1-[(2S)-2-amino-3,3-dimethyl-butanoyl]-4-(trifluoromethyl)pyrrolidine-2-carboxylate (Intermediate 15) as yellow oil.

MS obsd. (ESI⁺) [(M+H)⁺]: 387.2.

### Intermediate 16

### (2S)-2-(tert-butoxycarbonylamino)-2-(1-methylcyclopropyl)acetic acid

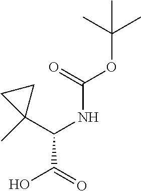 [SMILES: CC(C)(C)OC(=O)N[C@H](C(=O)O)C1(C)CC1]

### Step 1: Preparation of N-methoxy-N,1-dimethyl-cyclopropanecarboxamide

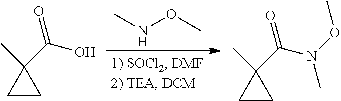

To a solution of 1-methylcyclopropane-1-carboxylic acid (3.1 g, 30.96 mmol) in DCM (10 mL) was added DMF (45 mg, 0.620 mmol) and SOCl₂ (3.7 g, 30.96 mmol) at 0° C. dropwise. The mixture was heated to 40° C. for 2 h. After cooled to room temperature, the mixture was added to a solution of Et₃N (934 mg, 92.36 mmol) and 1-methylcyclopropanecarbonyl chloride (3.65 g, 30.79 mmol) in DCM (50 mL) at 0° C. Then the mixture was stirred at 0-25° C. for 1 h. The mixture was concentrated and the residue was purified by silica gel column eluted with PE/EtOAc=20/1 to 4/1 to afford N-methoxy-N,1-dimethyl-cyclopropanecarboxamide (3.3 g) as yellow oil.

¹H NMR (400 MHz, CDCl₃) δ: ppm 3.72 (s, 3H), 3.23 (s, 3H), 1.36 (s, 3H), 1.06-1.00 (m, 2H), 0.59-0.53 (m, 2H).

### Step 2: Preparation of 1-methylcyclopropanecarbaldehyde

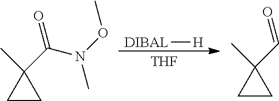

To a solution of N-methoxy-N,1-dimethyl-cyclopropanecarboxamide (6.0 g, 41.91 mmol) in THF (60 mL) was added DIBAL-H (50.29 mL, 50.29 mmol, 1 N) dropwise at −70° C. The mixture was stirred at −70° C. for 2 h. The mixture was poured into 1 N HCl (100 mL). The organic layer was separated and the aqueous phase was extracted with DCM (50 mL×3). The combined organic phase was dried over Na₂SO₄, filtered and concentrated to afford 1-methylcyclopropanecarbaldehyde solution which was used directly for next step without further purification.

¹H NMR (400 MHz, CDCl₃) δ: ppm 8.64 (s, 1H), 1.25 (s, 3H), 1.16-1.15 (m, 2H), 0.93-0.92 (m, 2H).

### Step 3: Preparation of (2S)-2-[[(1R)-2-hydroxy-1-phenyl-ethyl]amino]-2-(1-methylcyclopropyl)acetonitrile

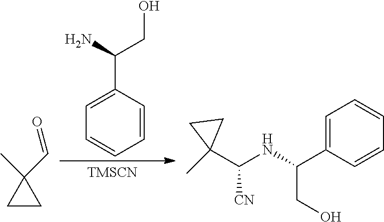

To a solution of 1-methylcyclopropanecarbaldehyde from step 2 was added (R)-(−)-2-phenylglycinol (4.11 g, 29.96 mmol). The mixture was stirred at 25° C. for 2 h. Then trimethylsilyl cyanide (4.95 g, 49.90 mmol) was added to the mixture dropwise at 0° C. Then the mixture was stirred at 0-25° C. for 12 h. The mixture was and the residue was purified by silica gel column eluted with PE/EtOAc=10/1 to 2/1 to afford (2S)-2-[[(1R)-2-hydroxy-1-phenyl-ethyl]amino]-2-(1-methylcyclopropyl)acetonitrile (5.1 g) as colorless oil.

MS obsd. (ESI⁺) [(M+H)⁺]: 231.1.

### Step 4: Preparation of (2S)-2-[[(1R)-2-hydroxy-1-phenyl-ethyl]amino]-2-(1-methylcyclopropyl)acetic acid

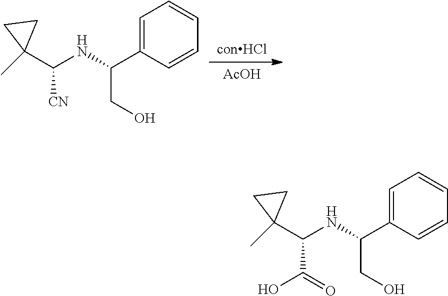 [SMILES: "AcOH" ; CC1([C@@H](C#N)N[C@@H](CO)c2ccccc2)CC1 ; CC1([C@H](N[C@@H](CO)c2ccccc2)C(=O)O)CC1]

To a solution of (2S)-2-[[(1R)-2-hydroxy-1-phenyl-ethyl]amino]-2-(1-methylcyclopropyl)acetonitrile (4.8 g, 20.84 mmol) in AcOH (10.0 mL) was added 12 N HCl (40.0 mL). The mixture was stirred at 80° C. for 1 h. The mixture was concentrated in vacuum. The crude product was purified by prep-HPLC (Column Phenomenex luna C₁₈ 250*80 mm*10 um; Condition ACN in water (HCl: 0%˜30%; 140 mL/min) to afford (2S)-2-[[(JR)-2-hydroxy-1-phenyl-ethyl]amino]-2-(1-methylcyclopropyl)acetic acid hydrochloride (2.7 g) as a white solid.

¹H NMR (400 MHz, D₂O) δ: ppm 7.53-7.40 (m, 5H), 4.20 (dd, J=8.0 Hz, 5.6 Hz, 1H), 4.07 (dd, J=12.0 Hz, 8.4 Hz, 1H), 4.00 (dd, J=12.0 Hz, 5.6 Hz, 1H), 2.57 (s, 1H), 0.98 (s, 3H), 0.60-0.35 (m, 3H), 0.05-0.15 (m, 1H).

MS obsd. (ESI⁺) [(M+H)⁺]: 249.8.

### Step 5: Preparation of (2S)-2-amino-2-(1-methylcyclopropyl)acetic acid

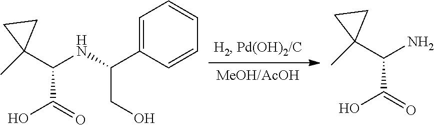

To a solution of (2S)-2-[[(1R)-2-hydroxy-1-phenyl-ethyl]amino]-2-(1-methylcyclopropyl)acetic acid; hydrochloride (2.7 g, 9.48 mmol) in AcOH (30 mL) and methanol (150 mL) was added wet Pd(OH)₂/C (200 mg, 10% purity). The suspension was degassed under vacuum and purged with H₂ for three times. The resulting mixture was stirred at 45° C. for 16 h under a H₂ balloon. The mixture was filtered and the filtrate was concentrated in vacuum to afford crude (2S)-2-amino-2-(1-methylcyclopropyl)acetic acid; hydrochloride (4.0 g) as yellow oil.

¹H NMR (400 MHz, D₂O) δ: ppm 3.07 (s, 1H), 1.20 (s, 3H), 0.96-0.79 (m, 1H), 0.57-0.53 (m, 2H), 0.52-0.46 (m, 1H).

MS obsd. (ESI⁺) [(M+H)⁺]: 130.3.

### Step 6: Preparation of (2S)-2-(tert-butoxycarbonylamino)-2-(1-methylcyclopropyl)acetic acid

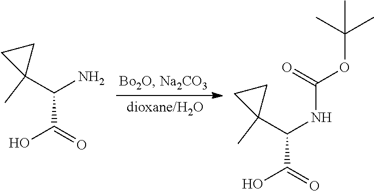

To a solution of (2S)-2-amino-2-(1-methylcyclopropyl)acetic acid; hydrochloride (4.0 g, 24.15 mmol, crude) in 1,4-dioxane (50 mL) and water (50 mL) was added Na₂CO₃ (12.8 g, 120.76 mmol) and di-t-butyldicarbonate (5271 mg, 24.15 mmol). The suspension was stirred at 25° C. for 12 h. The mixture was diluted with water (100 mL) and washed with EtOAc (50 mL×2). The aqueous phase was adjusted pH=4 with 1 N HCl and extracted with EtOAc (100 mL×2). The organic layers were washed with brine (60 mL), dried over Na₂SO₄ and concentrated in vacuum to afford (2S)-2-(tert-butoxycarbonylamino)-2-(1-methylcyclopropyl)acetic acid (1.9 g, Intermediate 16) as yellow oil.

¹H NMR (400 MHz, CDCl₃) δ: ppm 5.20 (d, J=3.6 Hz, 1H), 3.75 (d, J=7.6 Hz, 1H), 1.46 (s, 9H), 1.08 (s, 3H), 0.85-0.78 (m, 1H), 0.75-0.67 (m, 1H), 0.51-0.45 (m, 1H), 0.44-0.36 (m, 1H).

MS obsd. (ESI⁺) [(M-C₄H₉+H)⁺]: 174.1.

### Intermediate 17

### Benzyl (6S)-5-azaspiro[2.4]heptane-6-carboxylate hydrochloride

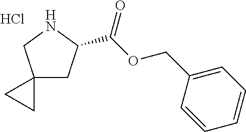 [SMILES: O=C(OCc1ccccc1)[C@@H]1CC2(CC2)CN1 ; Cl]

### Step 1: Preparation of 06-benzyl 05-tert-butyl (6S)-5-azaspiro[2.4]heptane-5,6-dicarboxylate

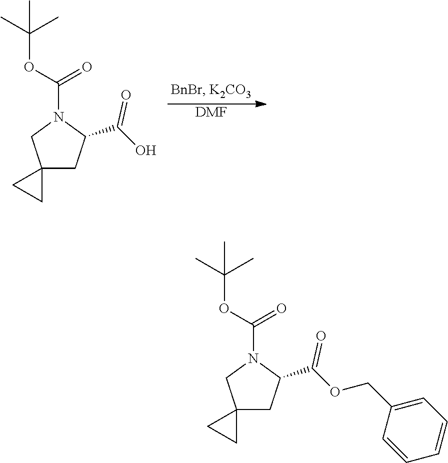

To a solution of (6S)-5-tert-butoxycarbonyl-5-azaspiro[2.4]heptane-6-carboxylic acid (2.00 g, 8.29 mmol) (Wuxiapptec, Cas number: 112963444-1) and K₂CO₃ (2.28 g, 16.55 mmol) in DMF (40 mL) was added BnBr (1.28 mL, 10.76 mmol). The mixture was stirred at 25° C. for 12 h. The mixture was diluted with water (100 mL) and extracted with EtOAc (100 mL×2). The combined organic phase was washed with brine (100 mL×2), dried with anhydrous Na₂SO₄, filtered and concentrated in vacuum. The residue was purified by silica gel chromatography eluted with ethyl PE/EtOAc=10/1 to 2/1 to afford O6-benzyl O5-tert-butyl (6S)-5-azaspiro[2.4]heptane-5,6-dicarboxylate (2300 mg) as colorless oil.

MS obsd. (ESI⁺) [(M-Boc+H)⁺]: 232.2.

### Step 2: Preparation of benzyl (6S)-5-azaspiro[2.4]heptane-6-carboxylate; hydrochloride

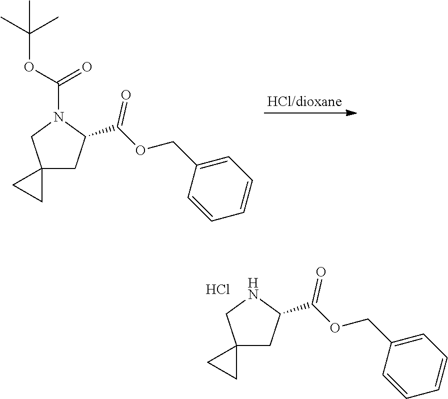 [SMILES: CC(C)(C)OC(=O)N1CC2(CC2)C[C@H]1C(=O)OCc1ccccc1 ; O=C(OCc1ccccc1)[C@@H]1CC2(CC2)CN1 ; Cl]

A solution of O6-benzyl O5-tert-butyl (6S)-5-azaspiro[2.4]heptane-5,6-dicarboxylate (2.3 g, 6.94 mmol) in 4 N HCl/dioxane (10.0 mL) was stirred at 25° C. for 1 h. The mixture was concentrated in vacuum to afford benzyl (6S)-5-azaspiro[2.4]heptane-6-carboxylate; hydrochloride (1800 mg, Intermediate 17) as yellow oil. MS obsd. (ESI⁺) [(M+H)⁺]: 232.2.

### Intermediate 18

### Benzyl (2S,4S)-1-[(2S)-2-amino-3,3-dimethyl-butanoyl]-4-methyl-pyrrolidine-2-carboxylate

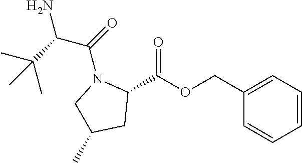 [SMILES: C[C@H]1C[C@@H](C(=O)OCc2ccccc2)N(C(=O)[C@@H](N)C(C)(C)C)C1]

The title compound was prepared in analogy to the procedure described for the preparation of Intermediate 1, by using (2S,4S)-1-tert-butoxycarbonyl-4-methyl-pyrrolidine-2-carboxylic acid (Bide, CAS number: 364750-81-2) instead of (1R,2S,5S)-3-tert-butoxycarbonyl-6,6-dimethyl-3-azabicyclo[3.1.0]hexane-2-carboxylic acid to afford benzyl (2S,4S)-1-[(2S)-2-amino-3,3-dimethyl-butanoyl]-4-methyl-pyrrolidine-2-carboxylate (Intermediate 18) as colorless oil.

MS obsd. (ESI⁺) [(M+H)⁺]: 333.2.

### Intermediate 19

### (2S)-3-(1-bicyclo[1.1.1]pentanyl)-2-(tert-butoxycarbonylamino)propanoic acid

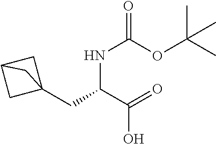 [SMILES: CC(C)(C)OC(=O)N[C@@H](CC12CC(C1)C2)C(=O)O]

### Step 1: Preparation of methyl (2S)-2-(tert-butoxycarbonylamino)-3-(3-iodo-1-bicyclo[1.1.1]pentanyl)propanoate

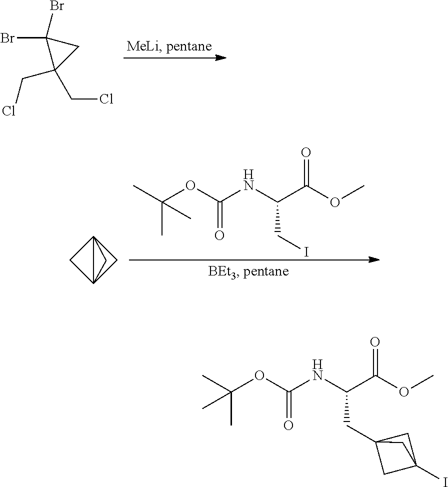 [SMILES: ClCC1(CCl)CC1(Br)Br ; C1C23CC12C3 ; COC(=O)[C@H](CI)NC(=O)OC(C)(C)C ; COC(=O)[C@H](CC12CC(I)(C1)C2)NC(=O)OC(C)(C)C]

To a solution of 1,1-dibromo-2,2-bis(chloromethyl)cyclopropane (30 g, 101.07 mmol) in pentane (30 mL) was added methyllithium (71.73 mL, 222.36 mmol) at −45° C. dropwise. The reaction was stirred at the same temperature for 15 min under N₂ protection. The reaction flask was removed from the dry ice/ethanol bath and placed in an ice-water bath. The reaction mixture was stirred at that temperature for 2 h. The volatiles were collected via a short path distillation condenser under a dry ice-ethanol environment at 50° C. to afford tricyclo[1.1.1.01,3]pentane (7.0 g) in dimethyl ether (˜50 mL). Boc-3-iodo-1-alanine methyl ester (7.0 g, 21.27 mmol) was added the solution and cooled to −40° C. Triethylborane (2.82 mL, 2.82 mmol) was added slowly. The resulting mixture was stirred at 20° C. for 1 h and then concentrated in vacuum. The residue was purified by reversed phase Flash (0.1% TFA in water, MeCN) to afford methyl (2S)-2-(tert-butoxycarbonylamino)-3-(3-iodo-1-bicyclo[1.1.1]pentanyl)propanoate (7.0 g) as a white solid.

¹H NMR (400 MHz, CDCl₃) δ: 4.98 (d, J=7.6 Hz, 1H), 4.30 (q, J=4.4 Hz, 1H), 3.74 (s, 3H), 2.23-2.28 (m, 6H), 2.10-2.16 (m, 1H), 1.84-1.90 (m, 1H), 1.45 (s, 9H).

MS obsd. (ESI⁺) [(M-Boc+H)⁺]: 295.9.

### Step 2: Preparation of methyl (2S)-3-(1-bicyclo[1.1.1]pentanyl)-2-(tert-butoxycarbonylamino)propanoate

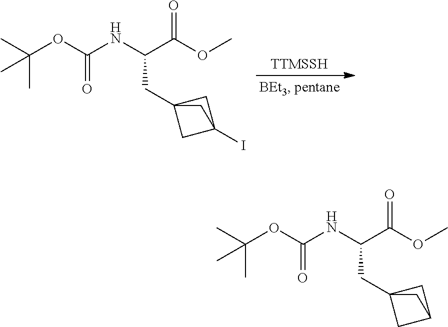 [SMILES: COC(=O)[C@H](CC12CC(I)(C1)C2)NC(=O)OC(C)(C)C ; COC(=O)[C@H](CC12CC(C1)C2)NC(=O)OC(C)(C)C]

To a solution of methyl (2S)-2-(tert-butoxycarbonylamino)-3-(3-iodo-1-bicyclo[1.1.1]pentanyl)propanoate (4.0 g, 10.12 mmol) and 2,6-lutidine (813 mg, 30.36 mmol) in pentane (30 mL) was added tris(trimethylsilyl)silane (6.24 mL, 20.24 mmol). The mixture was stirred for 20 min at 20° C., then BEt₃ (0.31 mL, 1.01 mmol) was added. The resulting mixture was stirred for 30 min at 20° C. The mixture was diluted with EtOAc (50 mL), washed with brine (50 mL), dried over Na₂SO₄, filtered and concentrated in vacuum. The residue was purified by silica gel column eluted with PE/EtOAc=50/1 to 8/1 to afford methyl (2S)-3-(1-bicyclo[1.1.1]pentanyl)-2-(tert-butoxycarbonylamino)propanoate (2.0 g) as colorless gum.

¹H NMR (400 MHz, CDCl₃) δ: 4.98 (d, J=7.6 Hz, 1H), 4.30 (q, J=4.4 Hz, 1H), 3.73 (s, 3H), 2.45 (s, 1H), 1.92-2.03 (m, 1H), 1.76-1.83 (m, 1H), 1.75 (s, 6H), 1.45 (s, 9H).

MS obsd. (ESI+) [M-Boc+H]+: 169.9.

### Step 3: Preparation of methyl (2S)-3-(1-bicyclo[1.1.1]pentanyl)-2-(tert-butoxycarbonylamino)propanoic acid

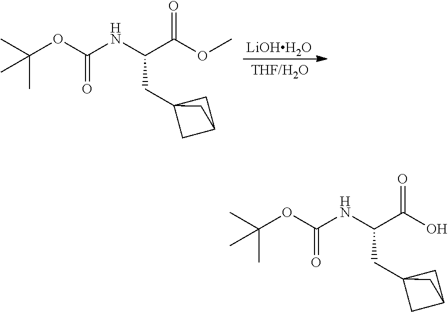

To a solution of methyl (2S)-3-(1-bicyclo[1.1.1]pentanyl)-2-(tert-butoxycarbonylamino)propanoate (1.03 g, 3.81 mmol) in THF (10 mL) and water (5 mL) was added LiOH·H₂O (480 mg, 11.43 mmol). The mixture was stirred at 25° C. for 4 h. The mixture was diluted with water (100 mL) and acidified with 1 N HCl (15 mL) to pH=4. The mixture was extracted with EtOAc (100 mL). The aqueous phase was extracted with EtOAc (50 mL). The combined organic phase was washed with brine (20 mL), dried over Na₂SO₄, filtered and concentrated to afford (2S)-3-(1-bicyclo[1.1.1]pentanyl)-2-(tert-butoxycarbonylamino)propanoic acid (802 mg, Intermediate 19) as yellow oil.

MS obsd. (ESI⁺) [(M-Boc+H)⁺]: 156.2.

### Intermediate 20

### (2S)-2-(benzyloxycarbonylamino)-4,4-dimethyl-pentanoic acid

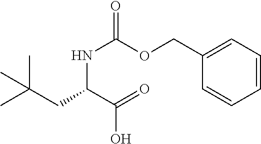 [SMILES: CC(C)(C)C[C@H](NC(=O)OCc1ccccc1)C(=O)O]

### Preparation of (2S)-2-(benzyloxycarbonylamino)-4,4-dimethyl-pentanoic acid

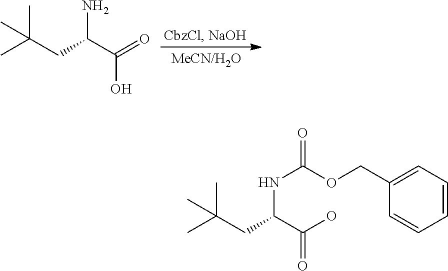

To a solution of (S)-2-(((benzyloxy)carbonyl)amino)-4,4-dimethylpentanoic acid (400 mg, 2.75 mmol) in aqueous solution of NaOH (5.52 mL, 1 N) was added a solution of CbzCl (0.48 mL, 3.38 mmol) in MeCN (0.80 mL) at 0° C. slowly over 5 min. The mixture was stirred at 20° C. for 12 h. The reaction was diluted with H₂O (15 mL) and acidified with 1 N HCl to pH=3. The mixture was extracted with EtOAc (30 mL×3). The organic phase was dried over Na₂SO₄, filtered and concentrated in vacuum. The residue was purified by silica gel column eluted with PE/EtOAc=2/1 to 1/1 to afford (2S)-2-(benzyloxycarbonylamino)-4,4-dimethyl-pentanoic acid (700 mg, Intermediate 20) as colorless oil.

MS obsd. (ESI⁺) [(M+H)⁺]: 280.1.

### Intermediate 21

### Benzyl (3S,3aS,6aR)-1,2,3,3a,4,5,6,6a-octahydrocyclopenta[c]pyrrole-3-carboxylate; 2,2,2-trifluoroacetic acid

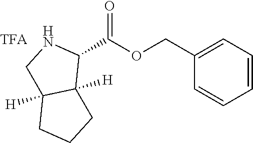 [SMILES: O=C(OCc1ccccc1)[C@H]1NC[C@@H]2CCC[C@@H]21]

The title compound was prepared in analogy to the procedure described for the preparation of the intermediate (1R,2S,5S)-benzyl 6,6-dimethyl-3-azabicyclo[3.1.0]hexane-2-carboxylate of Intermediate 1, by using (3S,3aS,6aR)-2-tert-butoxycarbonyl-3,3a,4,5,6,6a-hexahydro-1H-cyclopenta[c]pyrrole-3-carboxylic acid (AstaTech, CAS number: 597569-42-1) instead of (1R,2S,5S)-3-tert-butoxycarbonyl-6,6-dimethyl-3-azabicyclo[3.1.0]hexane-2-carboxylic acid to afford benzyl (3S,3aS,6aR)-1,2,3,3a,4,5,6,6a-octahydrocyclopenta[c]pyrrole-3-carboxylate; 2,2,2-trifluoroacetic acid (Intermediate 21) as yellow oil.

MS obsd. (ESI⁺) [(M+H)⁺]: 246.2.

### Intermediate 22

### Benzyl (3S,3aS,6aR)-2-[(2S,3R)-2-amino-3-methyl-pentanoyl]-3,3a,4,5,6,6a-hexahydro-1H-cyclopenta[c]pyrrole-3-carboxylate; 2,2,2-trifluoroacetic acid

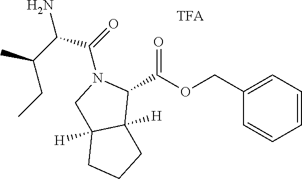 [SMILES: CC[C@@H](C)[C@H](N)C(=O)N1C[C@@H]2CCC[C@@H]2[C@H]1C(=O)OCc1ccccc1]

The title compound was prepared in analogy to the procedure described for the preparation of Intermediate 1, by using (3S,3aS,6aR)-2-tert-butoxycarbonyl-3,3a,4,5,6,6a-hexahydro-1H-cyclopenta[c]pyrrole-3-carboxylic acid instead of (1R,2S,5S)-3-tert-butoxycarbonyl-6,6-dimethyl-3-azabicyclo[3.1.0]hexane-2-carboxylic acid and Boc-L-isoleucine instead of (2S)-2-(tert-butoxycarbonylamino)-3,3-dimethyl-butanoic acid to afford benzyl (3S,3aS,6aR)-2-[(2S,3R)-2-amino-3-methyl-pentanoyl]-3,3a,4,5,6,6a-hexahydro-1H-cyclopenta[c]pyrrole-3-carboxylate; 2,2,2-trifluoroacetic acid (Intermediate 22) as yellow oil.

MS obsd. (ESI⁺) [(M+H)⁺]: 359.2.

### Intermediate 23

### Benzyl (3S,3aS,6aR)-2-[(2S)-2-amino-2-cyclobutyl-acetyl]-3,3a,4,5,6,6a-hexahydro-1H-cyclopenta[c]pyrrole-3-carboxylate; 2,2,2-trifluoroacetic acid

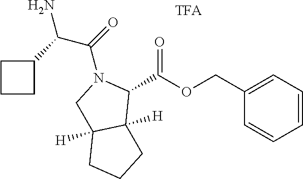 [SMILES: N[C@H](C(=O)N1C[C@@H]2CCC[C@@H]2[C@H]1C(=O)OCc1ccccc1)C1CCC1]

The title compound was prepared in analogy to the procedure described for the preparation of Intermediate 1, by using (3S,3aS,6aR)-2-tert-butoxycarbonyl-3,3a,4,5,6,6a-hexahydro-1H-cyclopenta[c]pyrrole-3-carboxylic acid instead of (1R,2S,5S)-3-tert-butoxycarbonyl-6,6-dimethyl-3-azabicyclo[3.1.0]hexane-2-carboxylic acid and (2S)-2-(tert-butoxycarbonylamino)-2-cyclobutyl-acetic acid (GL Biochem, CAS number: 155905-77-4) instead of (2S)-2-(tert-butoxycarbonylamino)-3,3-dimethyl-butanoic acid to afford benzyl (3S,3aS,6aR)-2-[(2S)-2-amino-2-cyclobutyl-acetyl]-3,3a,4,5,6,6a-hexahydro-1H-cyclopenta[c]pyrrole-3-carboxylate; 2,2,2-trifluoroacetic acid (Intermediate 23) as yellow oil.

MS obsd. (ESI⁺) [(M+H)⁺]: 357.2.

### Intermediate 24

### Benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid

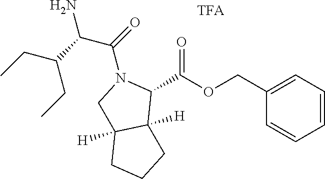 [SMILES: CCC(CC)[C@H](N)C(=O)N1C[C@@H]2CCC[C@@H]2[C@H]1C(=O)OCc1ccccc1]

The title compound was prepared in analogy to the procedure described for the preparation of Intermediate 1, by using (3S,3aS,6aR)-2-tert-butoxycarbonyl-3,3a,4,5,6,6a-hexahydro-1H-cyclopenta[c]pyrrole-3-carboxylic acid instead of (1R,2S,5S)-3-tert-butoxycarbonyl-6,6-dimethyl-3-azabicyclo[3.1.0]hexane-2-carboxylic acid and (2S)-2-(tert-butoxycarbonylamino)-3-ethyl-pentanoic acid (KaiXin Biological, CAS number: 35264-04-1) instead of (2S)-2-(tert-butoxycarbonylamino)-3,3-dimethyl-butanoic acid to afford benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) as yellow oil.

MS obsd. (ESI⁺) [(M+H)⁺]: 373.2.

### Intermediate 25

### (2S)-2-(tert-butoxycarbonylamino)-3,3-dimethyl-pentanoic acid

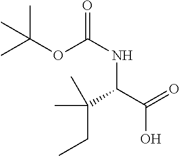 [SMILES: CCC(C)(C)[C@H](NC(=O)OC(C)(C)C)C(=O)O]

### Step 1: Preparation of N-methoxy-N,2,2-trimethyl-butanamide

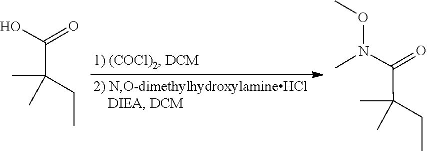

To a solution of 2, 2-dimethylbutyric acid (15.0 g, 129.13 mmol) in DCM (50 mL) was added (COCl)₂ (32.78 g, 258.26 mmol) at 0° C. The mixture was stirred at 20° C. for 8 h and then concentrated in vacuum at 45° C. The residue was dissolve in DCM (30 mL) and added to a solution of O, N-dimethylhydroxylamine HCl (12.6 g, 129.13 mmol) and Et₃N (65.33 g, 645.66 mmol) in DCM (300 mL) at 0° C. Then the mixture was stirred at 20° C. for 4 h. The reaction mixture was diluted with water (200 mL) and extracted with DCM (200 mL×2). The organic phase was washed with 1 N HCl (200 mL), brine (50 mL), dried over Na₂SO₄, filtered and concentrated. The residue was purified by silica gel column eluted with PE to PE/EtOAc=6/1 to afford N-methoxy-N, 2, 2-trimethyl-butanamide (15.5 g) as yellow oil.

¹H NMR (400 MHz, CDCl₃) δ: ppm 3.67 (s, 3H), 3.18 (s, 3H), 1.66 (q, J=7.6 Hz, 2H), 1.22 (s, 6H), 0.85 (t, J=7.6 Hz, 3H).

MS obsd. (ESI⁺) [(M+H)⁺]: 160.1.

### Step 2: Preparation of 2,2-dimethylbutanal

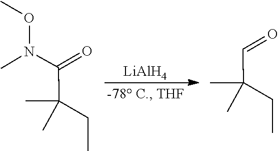

To a solution of N-methoxy-N,2,2-trimethyl-butanamide (5.0 g, 31.4 mmol) in THF (50 mL) was added LiAlH₄/THF (47.1 mL, 47.1 mmol, 1 N) at −78° C. and stirred for 1 h at the same temperature. The reaction was quenched with H₂O (1.8 mL) dropwise at −78° C. in 10 min, and then a aqueous solution of NaOH (1.8 mL, 15%) was added at −78° C. in 5 min. Then H₂O (5.5 mL) was added at −78° C. in 5 min. The white suspension was warmed to 15° C. and stirred for 10 min. After the addition of Na₂SO₄ (10 g), the mixture was stirred at 15° C. for another 10 min. The mixture was filtered and the filter cake was washed with THF (25 mL×2). The filtrate was used directly for next step.

### Step 3: Preparation of 2-[[(JR)-2-hydroxy-1-phenyl-ethyl]amino]-3,3-dimethyl-pentanenitrile

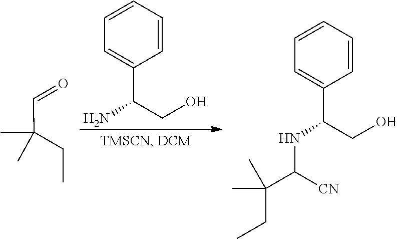

To a solution of 2, 2-dimethylbutanal (3.0 g, 29.95 mmol) in THF (100 mL) was added (R)-(−)-2-phenylglycinol (4.11 g, 29.95 mmol). The reaction was stirred at 15° C. for 2 h. After cooled to 0° C., trimethylsilyl cyanide (5.94 g, 59.9 mmol) was added at 0° C. dropwise in 15 min. The resulting mixture was stirred at 20° C. for 12 h. The reaction mixture was concentrated and the residue was purified by silica gel column eluted with PE to PE/EtOAc=4/1 to afford 2-[[(1R)-2-hydroxy-1-phenyl-ethyl]amino]-3, 3-dimethyl-pentanenitrile (5.0 g) as yellow oil.

MS obsd. (ESI⁺) [(M+H)⁺]: 247.2.

### Step 4: Preparation of (2S)-2-[[(1R)-2-hydroxy-1-phenyl-ethyl]amino]-3,3-dimethyl-pentanoic acid

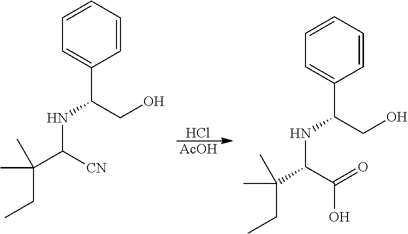 [SMILES: "AcOH" ; CCC(C)(C)C(C#N)N[C@@H](CO)c1ccccc1 ; Cl ; CCC(C)(C)[C@H](N[C@@H](CO)c1ccccc1)C(=O)O]

To a solution of 2-[[(1R)-2-hydroxy-1-phenyl-ethyl]amino]-3,3-dimethyl-pentanoic acid (5.0 g, 18.84 mmol) in acetic acid (10 mL) was added con. HCl (20.0 mL). The reaction was stirred at 80° C. for 12 h. The reaction solution was concentrated and the residue was purified by Prep-HPLC (Phenomenex luna C18 (250*70 mm, 10 um), water (0.1% HCl)-CAN, 20-30%, 140 ml/min) to afford (2S)-2-[[(1R)-2-hydroxy-1-phenyl-ethyl]amino]-3,3-dimethyl-pentanoic acid (2.5 g) as a white solid.

¹H NMR (400 MHz, DMSO-d₆) δ: ppm 7.40-7.32 (m, 5H), 5.31-5.05 (m, 1H), 3.69-3.52 (m, 3H), 2.77 (s, 1H), 1.39-1.24 (m, 2H), 0.88-0.81 (m, 6H), 0.57 (t, J=7.6 Hz, 3H).

MS obsd. (ESI⁺) [(M+H)⁺]: 266.1.

### Step 5: Preparation of (2S)-2-amino-3,3-dimethyl-pentanoic acid

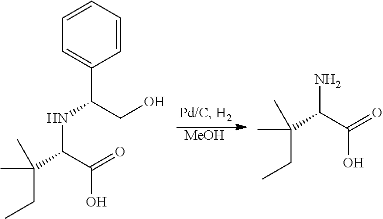

To a solution of (2S)-2-[[(1R)-2-hydroxy-1-phenyl-ethyl]amino]-3,3-dimethyl-pentanoic acid (2.5 g, 9.42 mmol) in methanol (60 mL) and acetic acid (40 mL) was added Pd(OH)₂ (750 mg). The suspension was degassed with H₂ for 3 times and stirred for 16 h at 20° C. under a H₂ balloon. The suspension was filtered and the filtrate was concentrated in vacuum to afford (2S)-2-amino-3,3-dimethyl-pentanoic acid; acetic acid (1.93 g) as a white solid.

MS obsd. (ESI⁺) [(M+H)⁺]: 146.2.

### Step 6: Preparation of (2S)-2-(tert-butoxycarbonylamino)-3,3-dimethyl-pentanoic acid

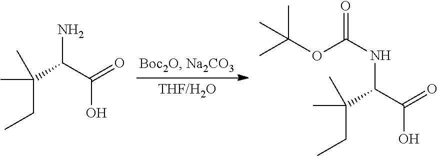

To a solution of (2S)-2-amino-3,3-dimethyl-pentanoic acid; acetic acid (1.78 g, 8.67 mmol) in THF (40 mL) and water (40 mL) was added Na₂CO₃ (4.59 g, 43.33 mmol) and (Boc)₂O (2.84 g, 12.99 mmol) successively. The suspension was stirred at 20° C. for 12 h. The suspension was diluted with H₂O (40 mL) and extracted with EtOAc (50 mL×3). The aqueous phase was acidified with 1 N HCl to pH=4 and extracted with EtOAc (50 mL×3). The organic phase was dried over anhydrous Na₂SO₄, filtered and concentrated in vacuum to afford (2S)-2-(tert-butoxycarbonylamino)-3,3-dimethyl-pentanoic acid (1.8 g, Intermediate 25) as colorless oil.

¹H NMR (400 MHz, CDCl₃) δ: ppm 5.05-5.04 (m, 1H), 4.21 (d, J=9.2 Hz, 1H), 2.11 (s, 1H), 1.46 (s, 9H), 1.50 (q, J=7.2 Hz, 2H), 0.97 (s, 6H), 0.91 (t, J=7.2 Hz, 3H).

MS obsd. (ESI⁺) [(M+Na)⁺]: 268.1.

### Intermediate 26

### Benzyl N-amino-N-(3-amino-3-oxo-propyl)carbamate

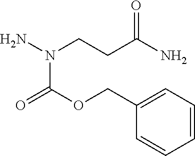 [SMILES: NC(=O)CCN(N)C(=O)OCc1ccccc1]

### Step 1: Preparation of tert-butyl N-[(3-amino-3-oxo-propyl)amino]carbamate

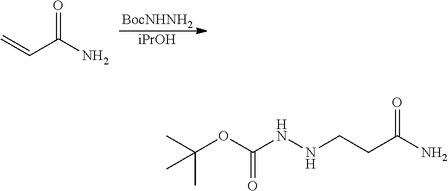

To a solution of tert-butyl hydrazinecarboxylate (10.0 g, 75.67 mmol) in iPrOH (100 mL) was added acrylamide (5.38 g, 75.67 mmol). The reaction mixture was stirred at 70° C. for 3 h. The mixture was concentrated and the residue was dissolved in ethyl acetate (60 mL), washed with 1 N HCl (30 mL), brine (20 mL) and dried over anhydrous Na₂SO₄. After filtration and concentration, tert-butyl 2-(3-amino-3-oxopropyl)hydrazine-1-carboxylate (8 g) was obtained as colorless oil.

### Step 2: Preparation of benzyl N-(3-amino-3-oxo-propyl)-N-(tert-butoxycarbonylamino)carbamate

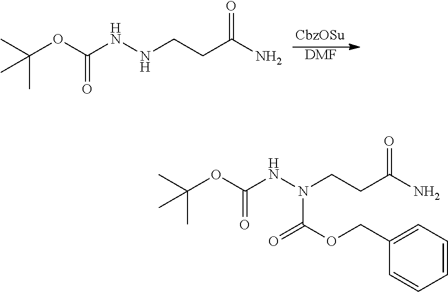

To a solution of tert-butyl 2-(3-amino-3-oxopropyl)hydrazine-1-carboxylate (1.2 g, 5.9 mmol) was added benzyl (2,5-dioxopyrrolidin-1-yl) carbonate (1.47 g, 5.9 mmol) in DMF (30 mL) at 25° C. The mixture was stirred at 25° C. for 12 h. The reaction mixture was diluted with H₂O (100 mL) and extracted with EtOAc (100 mL×3). The organic phase was washed with brine (80 mL×3), dried over Na₂SO₄, concentrated in vacuum. The residue was purified by silica gel column eluted with DCM to DCM/MeOH=10/1 to afford benzyl N-(3-amino-3-oxo-propyl)-N-(tert-butoxycarbonylamino)carbamate (580 mg) as a white solid.

MS obsd. (ESI⁺) [(M+Na)⁺]: 360.1.

### Step 3: Preparation of benzyl N-amino-N-(3-amino-3-oxo-propyl)carbamate; 2,2,2-trifluoroacetic acid

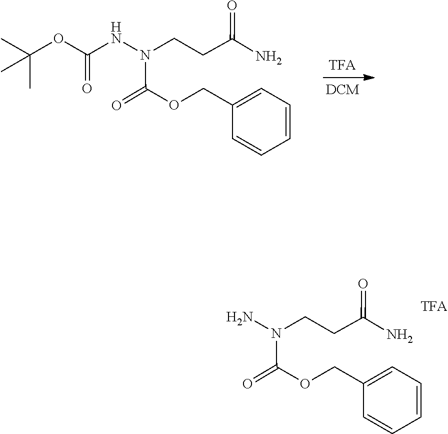 [SMILES: CC(C)(C)OC(=O)NN(CCC(N)=O)C(=O)OCc1ccccc1 ; NC(=O)CCN(N)C(=O)OCc1ccccc1]

To a solution of benzyl N-(3-amino-3-oxo-propyl)-N-(tert-butoxycarbonylamino)carbamate (500 mg, 1.48 mmol) in DCM (3 mL) was added TFA (3 mL). The reaction mixture was stirred at 20° C. for 1 h. The mixture was concentrated in vacuum to afford benzyl N-amino-N-(3-amino-3-oxo-propyl)carbamate 2,2,2-trifluoroacetic acid (520 mg, Intermediate 26) as yellow oil which was used directly for the next step.

### Intermediate 27

### Trans-methyl 1,2,3,3a,4,5,6,6a-octahydrocyclopenta[c]pyrrole-3-carboxylate

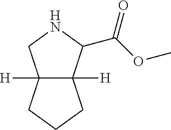

### Step 1: Preparation of 1,3a,4,5,6,6a-hexahydrocyclopenta[c]pyrrole

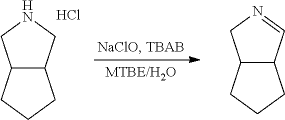 [SMILES: C1CC2CNCC2C1 ; Cl ; C1=NCC2CCCC12]

To a stirred solution of 13% NaClO/H₂O (11.78 mL) and MTBE (10 mL) was added 3-azabicyclo[3.3.0]octane hydrochloride (2.0 g, 13.55 mmol) (Accela, CAS number: 112626-50-3) at 0° C. Then the reaction mixture was stirred at 0° C. for 3 h. The reaction mixture was warmed to room temperature and diluted with MTBE (20 mL). The organic phase was added to a stirred solution of 25% wt. aqueous NaOH/H₂O (8.64 mL) and TBAB (280.0 mg, 0.870 mmol). The mixture stirred at room temperature for 1 h and at 50° C. for 48 h. The reaction mixture was cooled to room temperature and washed with 20% brine (10 mL). The organic phase was dried over anhydrous Na₂SO₄, filtered and concentrated in vacuum to afford 1,3a,4,5,6,6a-hexahydrocyclopenta[c]pyrrole (1.23 g) as a white solid.

¹H NMR (400 MHz, CDCl₃) δ: ppm 7.31 (s, 1H), 4.10-4.03 (m, 1H), 3.57-3.51 (m, 1H), 3.29 (t, J=8.0 Hz, 1H), 2.71-2.63 (m, 1H), 1.70-1.65 (m, 3H), 1.60-1.54 (m, 1H), 1.42-1.38 (m, 1H), 1.30-1.26 (m, 1H).

### Step 2: Preparation of trans-1,2,3,3a,4,5,6,6a-octahydrocyclopenta[c]pyrrole-3-carbonitrile

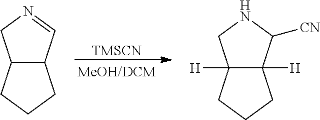 [SMILES: "TMSCN" ; C1=NCC2CCCC12 ; N#CC1NCC2CCCC21]

To a solution of 1,3a,4,5,6,6a-hexahydrocyclopenta[c]pyrrole (1.23 g, 11.27 mmol) in DCM (10 mL) was added methanol (1.23 mL) slowly, followed by TMSCN (2.79 g, 28.17 mmol) at 0° C. The mixture was stirred at 10° C. for 3 h. The reaction mixture was concentrated in vacuum and the residue was purified by silica gel column eluted with PE to PE/EtOAc=1/1 to afford trans-1,2,3,3a,4,5,6,6a-octahydrocyclopenta[c]pyrrole-3-carbonitrile (1.2 g) as yellow oil.

¹H NMR (400 MHz, CDCl₃) δ: ppm 3.67 (d, J=2.4 Hz, 1H), 3.24 (dd, J=10.0 Hz, 7.6 Hz, 1H), 2.83-2.76 (m, 2H), 2.72-2.64 (m, 1H), 1.99-1.82 (m, 3H), 1.70-1.61 (m, 1H), 1.49-1.43 (m, 1H), 1.41-1.33 (m, 2H).

### Step 3: Preparation of trans-methyl 1,2,3,3a,4,5,6,6a-octahydrocyclopenta[c]pyrrole-3-carboxylate

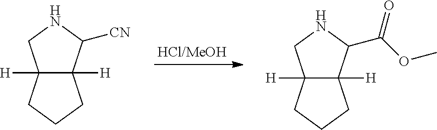

A solution of (3S,3aS,6aR)-1,2,3,3a,4,5,6,6a-octahydrocyclopenta[c]pyrrole-3-carbonitrile (1.2 g, 8.81 mmol) in HCl/MeOH (11.0 mL, 4 N) was stirred for 2 h at 25° C. The reaction mixture was concentrated in vacuum to remove HCl/MeOH. The residue was diluted with EtOAc (80 mL) and washed with saturated aqueous solution of Na₂CO₃ (40 mL). The aqueous layer was extracted with EtOAc (50 mL×5). The combined organic phase was washed with brine (30 mL), dried over anhydrous Na₂SO₄, filtered and concentrated in vacuum to afford trans-methyl 1,2,3,3a,4,5,6,6a-octahydrocyclopenta[c]pyrrole-3-carboxylate (1.1 g, Intermediate 27) as yellow oil.

¹H NMR (400 MHz, CDCl₃) δ: ppm 3.73 (s, 3H), 3.31-3.27 (m, 2H), 2.62-2.57 (m, 2H), 2.52-2.48 (m, 1H), 2.15 (br s, 1H), 1.78-1.74 (m, 1H), 1.67-1.56 (m, 4H), 1.44-1.38 (m, 1H).

### Intermediate 28

### (2S)-2-chloro-2-fluoro-acetyl chloride

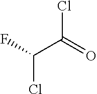

### Step 1: Preparation of (2S)-2-chloro-2-fluoro-acetic acid

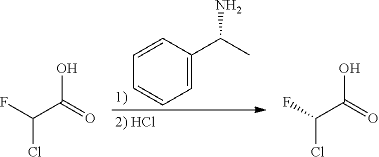

To a solution of 2-chloro-2-fluoro-acetic acid (160.0 g, 1.42 mol) in EtOAc (600 mL) was added (R)-1-phenylethanamine (186.1 g, 1.54 mol) in EtOAc (600 mL) at 0° C. The reaction was stirred at 0° C. for 2 h. The reaction mixture was filtered and the filter cake was triturated in acetone (720 g, 2.2 L) at 80° C. for 1 h. The resulting solution was slowly cooled to 30° C. and stirred at 30° C. for 16 hous. The trituration was repeated for four times. Optical Rotation (C=3.8 g/100 mL in MeOH at 25° C., salt) showed the specific rotation was +10.896. The suspension was filtered, filter cake dissolved in water (1 L), and then acidified with 1N HCl (1.5 L). The mixture was extracted with MTBE (500 mL×10). The combined organic phase was dried over Na₂SO₄, filtered and concentrated to afford (2S)-2-chloro-2-fluoro-acetic acid (138.88 g) as a brown liquid.

¹H NMR (400 MHz, CDCl₃) δ: ppm 10.12 (s, 1H), 6.26 (d, J=50.4 Hz, 1H).

### Step 2: Preparation of (2S)-2-chloro-2-fluoro-acetyl chloride

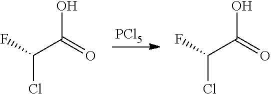

To PCl₅ (28.5 g, 136.65 mmol) was added (2S)-2-chloro-2-fluoro-acetic acid (21.3 g, 124.02 mmol) dropwise at 0° C. under N₂ with stirring (the internal temperature of the suspension did not over 5° C.) for 1 h. The suspension was warmed to 25° C. for 30 min and stirred at 25° C. for 1 h to give a clear solution. The solution was heated to 70° C. and stirred at 70° C. for 1 h. ¹H-NMR showed starting material was consumed completely and desired product was formed. The mixture was purified by distillation (70° C., 30 mmHg) to obtained (2S)-2-chloro-2-fluoro-acetyl chloride (42.0 g, Intermediate 28) as colorless liquid.

¹H NMR (400 MHz, CDCl₃) δ: ppm 6.47-6.24 (m, 1H).

### Alternative Synthetic Method

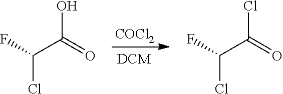

To a solution of (2S)-2-chloro-2-fluoro-acetic acid (1478 mg, 7.89 mmol, 60% purity) in DCM (15 mL) was added oxalyl chloride (841 mg, 6.62 mmol) and 5 drops of DMF at 0° C. Then the mixture was stirred at 25° C. for 1 hr. The solution was used for next step without further purification.

### Intermediate 29

### Methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid

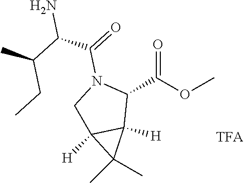 [SMILES: CC[C@@H](C)[C@H](N)C(=O)N1C[C@H]2[C@@H]([C@H]1C(=O)OC)C2(C)C]

### Step 1: Preparation of methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate

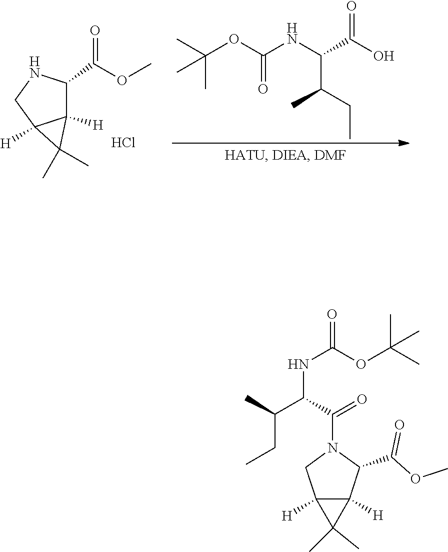

To a solution of Boc-L-isoleucine (25.0 g, 108.09 mmol) in DMF (200 mL) was added DIPEA (69.88 g, 540.62 mmol). After cooling to 0° C., to the mixture was added HATU (51.5 g, 135.44 mmol) and methyl (1R,2S,5S)-6,6-dimethyl-3-azabicyclo[3.1.0]hexane-2-carboxylate; hydrochloride (23.37 g, 113.65 mmol). Then the reaction mixture was stirred at 15° C. for 12 h. The reaction mixture was poured into water (300 mL) and extracted with EtOAc (300 mL×3). The combined organic phase was washed with brine (300 mL), dried over Na₂SO₄, filtered and concentrated in vacuum. The residue was purified by silica gel column eluted with PE/EtOAc=5/1 to afford methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate (35.0 g) as yellow oil.

MS obsd. (ESI⁺) [(M+H)⁺]: 383.2.

### Step 2: Preparation of methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid

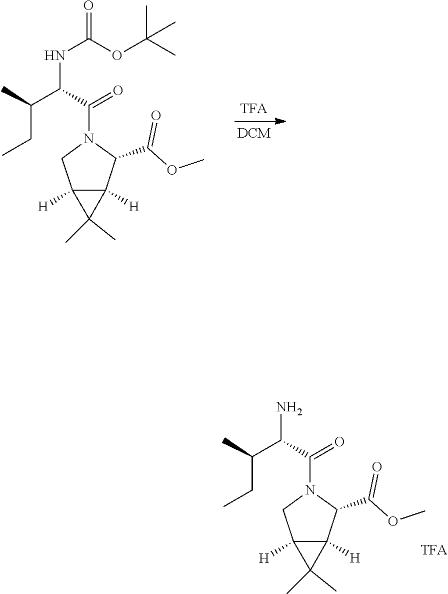 [SMILES: CC[C@@H](C)[C@H](NC(=O)OC(C)(C)C)C(=O)N1C[C@H]2[C@@H]([C@H]1C(=O)OC)C2(C)C ; CC[C@@H](C)[C@H](N)C(=O)N1C[C@H]2[C@@H]([C@H]1C(=O)OC)C2(C)C]

To a solution of methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate (5.0 g, 13.07 mmol) in DCM (30 mL) was added TFA (30.0 mL). The reaction mixture was stirred at 25° C. for 30 min. The reaction mixture was concentrated in vacuum to afford methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid (5.0 g, Intermediate 29) as yellow oil.

MS obsd. (ESI⁺) [(M+H)⁺]: 283.4.

### Intermediate 30

### Methyl (1R,2S,5S)-3-[(2S)-2-amino-3-ethyl-pentanoyl]-6,6-dimethyl-3-azabicyclo[3.1.0]hexane-2-carboxylate; 2,2,2-trifluoroacetic acid

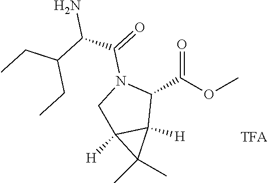 [SMILES: CCC(CC)[C@H](N)C(=O)N1C[C@H]2[C@@H]([C@H]1C(=O)OC)C2(C)C]

The title compound was prepared in analogy to the procedure described for the preparation of Intermediate 29, by using (2S)-2-(tert-butoxycarbonylamino)-3-ethyl-pentanoic acid (KaiXin Biological, CAS number: 35264-04-1) instead of Boc-L-isoleucine to afford methyl (1R,2S,5S)-3-[(2S)-2-amino-3-ethyl-pentanoyl]-6,6-dimethyl-3-azabicyclo[3.1.0]hexane-2-carboxylate; 2,2,2-trifluoroacetic acid (Intermediate 30) as yellow oil.

MS obsd. (ESI⁺) [(M+H)⁺]: 297.0.

### Intermediate 31

### Benzyl (1R,2S,5S)-3-[(2S)-2-amino-3-ethyl-pentanoyl]-6,6-dimethyl-3-azabicyclo[3.1.0]hexane-2-carboxylate; hydrochloride

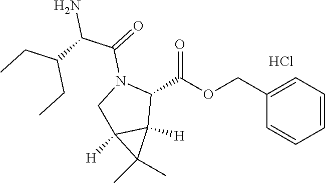 [SMILES: CCC(CC)[C@H](N)C(=O)N1C[C@H]2[C@@H]([C@H]1C(=O)OCc1ccccc1)C2(C)C ; Cl]

The title compound was prepared in analogy to the procedure described for the preparation of Intermediate 1, by using (2S)-2-(tert-butoxycarbonylamino)-3-ethyl-pentanoic acid (KaiXin Biological, CAS number: 35264-04-1) instead of (2S)-2-(tert-butoxycarbonylamino)-3,3-dimethyl-butanoic acid to afford benzyl (1R,2S,5S)-3-[(2S)-2-amino-3-ethyl-pentanoyl]-6,6-dimethyl-3-azabicyclo[3.1.0]hexane-2-carboxylate; hydrochloride (Intermediate 31) as yellow oil.

MS obsd. (ESI⁺) [(M+H)⁺]: 373.4.

### Intermediate 32

### Benzyl (2S)-1-[(2S)-2-amino-3-ethyl-pentanoyl]-4,4-dimethyl-pyrrolidine-2-carboxylate; hydrochloride

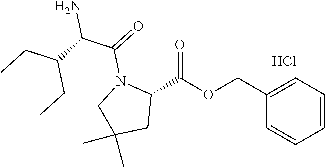 [SMILES: CCC(CC)[C@H](N)C(=O)N1CC(C)(C)C[C@H]1C(=O)OCc1ccccc1 ; Cl]

The title compound was prepared in analogy to the procedure described for the preparation of Intermediate 1, by using (2S)-4,4-dimethylpyrrolidine-2-carboxylic acid; hydrochloride (Porse Fine Chemical, CAS number: 1443252-76-3) instead of (1R,2S,5S)-6,6-dimethyl-3-azabicyclo[3.1.0]hexane-2-carboxylic acid; hydrochloride, and (2S)-1-tert-butoxycarbonyl-4,4-dimethyl-pyrrolidine-2-carboxylic acid instead of (1R,2S,5S)-3-tert-butoxycarbonyl-6,6-dimethyl-3-azabicyclo[3.1.0]hexane-2-carboxylic acid to afford benzyl (2S)-1-[(2S)-2-amino-3-ethyl-pentanoyl]-4,4-dimethyl-pyrrolidine-2-carboxylate; hydrochloride (Intermediate 32) as yellow oil.

MS obsd. (ESI⁺) [(M+H)⁺]: 361.3.

### Intermediate 33

### (2S)-2-(benzyloxycarbonylamino)-3,3-dimethyl-pentanoic acid

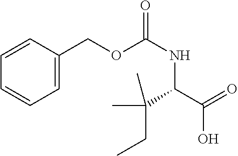 [SMILES: CCC(C)(C)[C@H](NC(=O)OCc1ccccc1)C(=O)O]

### Preparation of (2S)-2-(benzyloxycarbonylamino)-3,3-dimethyl-pentanoic acid

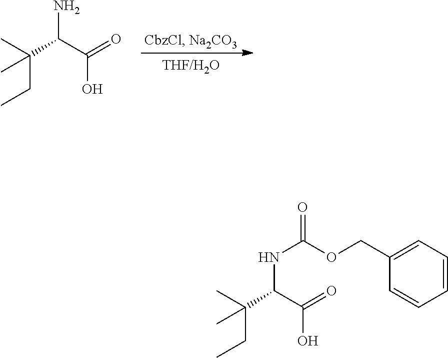

To a solution of (2S)-2-amino-3,3-dimethyl-pentanoic acid; acetic acid (900 mg, 4.38 mmol, from Intermediate 25, step 5) in water (10 mL) and THF (10 mL) was added Na₂CO₃ (1.4 g, 13.21 mmol). After cooling to 0° C., to the mixture was added CbzCl (1.13 g, 6.62 mmol). The reaction mixture was stirred at 20° C. for 12 h. The suspension was diluted with H₂O (20 mL) and extracted with EtOAc (40 mL). The aqueous phase was acidified with 1N HCl to pH=2 and extracted with EtOAc (40 mL×3). The organic phase was dried with Na₂SO₄, filtered and concentrated in vacuum. The residue was purified by reverse flash (condition: 120 g Flash Column Welch Ultimate XB_C18 20-40 μm; 120 A, water (0.1% TFA)-ACN, 30-60%, 65 ml/min) to afford (2S)-2-(benzyloxycarbonylamino)-3,3-dimethyl-pentanoic acid (440.0 mg) as colorless oil.

MS obsd. (ESI⁺) [(M+H)⁺]: 280.1.

### Intermediate 34

### Benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate; hydrochloride

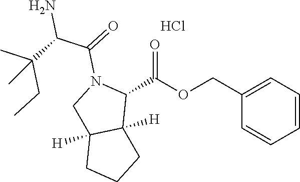 [SMILES: CCC(C)(C)[C@H](N)C(=O)N1C[C@@H]2CCC[C@@H]2[C@H]1C(=O)OCc1ccccc1 ; Cl]

The title compound was prepared in analogy to the procedure described for the preparation of Intermediate 1, by using (3S,3aS,6aR)-2-tert-butoxycarbonyl-3,3a,4,5,6,6a-hexahydro-1H-cyclopenta[c]pyrrole-3-carboxylic acid instead of (1R,2S,5S)-3-tert-butoxycarbonyl-6,6-dimethyl-3-azabicyclo[3.1.0]hexane-2-carboxylic acid, and (2S)-2-(tert-butoxycarbonylamino)-3,3-dimethyl-pentanoic acid (Intermediate 25) instead of (2S)-2-(tert-butoxycarbonylamino)-3,3-dimethyl-butanoic acid to afford benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) as colorless oil.

MS obsd. (ESI⁺) [(M+H)⁺]: 373.2.

### Intermediate 35

### Benzyl (2S)-1-[(2S)-2-amino-3,3-dimethyl-pentanoyl]-4,4-dimethyl-pyrrolidine-2-carboxylate; hydrochloride

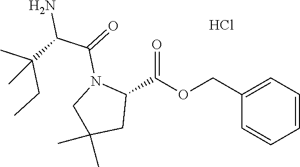 [SMILES: CCC(C)(C)[C@H](N)C(=O)N1CC(C)(C)C[C@H]1C(=O)OCc1ccccc1 ; Cl]

The title compound was prepared in analogy to the procedure described for the preparation of Intermediate 1, by using (2S)-4,4-dimethylpyrrolidine-2-carboxylic acid; hydrochloride (Porse Fine Chemical, CAS number: 1443252-76-3) instead of (1R,2S,5S)-6,6-dimethyl-3-azabicyclo[3.1.0]hexane-2-carboxylic acid; hydrochloride, and (2S)-2-(tert-butoxycarbonylamino)-3,3-dimethyl-pentanoic acid (Intermediate 25) instead of (2S)-2-(tert-butoxycarbonylamino)-3,3-dimethyl-butanoic acid (BePharm, CAS number: 62965-35-9) to afford benzyl (2S)-1-[(2S)-2-amino-3,3-dimethyl-pentanoyl]-4,4-dimethyl-pyrrolidine-2-carboxylate; hydrochloride (Intermediate 35) as yellow oil.

MS obsd. (ESI⁺) [(M+H)⁺]: 361.2.

### Intermediate 36

### Benzyl 1-[(2S)-2-amino-3,3-dimethyl-butanoyl]-3,3-dimethyl-pyrrolidine-2-carboxylate; 2,2,2-trifluoroacetic acid

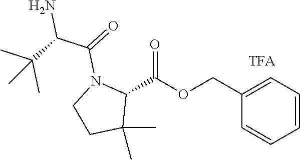 [SMILES: CC(C)(C)[C@H](N)C(=O)N1CCC(C)(C)[C@H]1C(=O)OCc1ccccc1 ; "TFA"]

The title compound was prepared in analogy to the procedure described for the preparation of Intermediate 1, by using 3,3-dimethylpyrrolidine-2-carboxylic acid; hydrochloride (Chengdu AstaTech, CAS number: 61406-78-8) instead of (1R,2S,5S)-6,6-dimethyl-3-azabicyclo[3.1.0]hexane-2-carboxylic acid; hydrochloride to afford benzyl 1-[(2S)-2-amino-3,3-dimethyl-butanoyl]-3,3-dimethyl-pyrrolidine-2-carboxylate; 2,2,2-trifluoroacetic acid (Intermediate 36) as yellow oil.

MS obsd. (ESI⁺) [(M+H)⁺]: 347.2.

### Intermediate 37

### (1R,2S,5S)-3-[(2S)-2-amino-2-cyclobutyl-acetyl]-6,6-dimethyl-3-azabicyclo[3.1.0]hexane-2-carboxylate; 2,2,2-trifluoroacetic acid

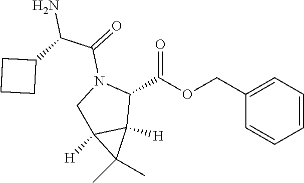 [SMILES: CC1(C)[C@@H]2[C@@H](C(=O)OCc3ccccc3)N(C(=O)[C@@H](N)C3CCC3)C[C@@H]21]

The title compound was prepared in analogy to the procedure described for the preparation of Intermediate 1, by using (2S)-2-(tert-butoxycarbonylamino)-2-cyclobutyl-acetic acid (GL Biochem, CAS number: 155905-77-4) instead of (2S)-2-(tert-butoxycarbonylamino)-3,3-dimethyl-butanoic acid to afford (1R,2S,5S)-3-[(2S)-2-amino-2-cyclobutyl-acetyl]-6,6-dimethyl-3-azabicyclo[3.1.0]hexane-2-carboxylate; 2,2,2-trifluoroacetic acid (Intermediate 37) as yellow oil.

MS obsd. (ESI⁺) [(M+H)⁺]: 357.2.

### Intermediate 38

### Benzyl (2S,4R)-1-[(2S,3S)-2-amino-3-methyl-pentanoyl]-4-(trifluoromethyl)pyrrolidine-2-carboxylate; 2,2,2-trifluoroacetaldehyde

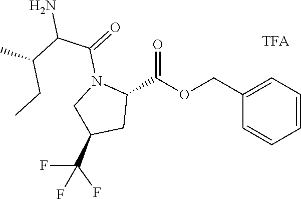 [SMILES: CC[C@H](C)C(N)C(=O)N1C[C@H](C(F)(F)F)C[C@H]1C(=O)OCc1ccccc1]

The title compound was prepared in analogy to the procedure described for the preparation of Intermediate 1, by using (2S,4R)-1-tert-butoxycarbonyl-4-(trifluoromethyl)pyrrolidine-2-carboxylic acid (PharmaBlock, CAS number: 470482-44-1) instead of (1R,2S,5S)-3-tert-butoxycarbonyl-6,6-dimethyl-3-azabicyclo[3.1.0]hexane-2-carboxylic acid, and Boc-Ile-OH instead of (2S)-2-(tert-butoxycarbonylamino)-3,3-dimethyl-butanoic acid to afford benzyl (2S,4R)-1-[(2S,3S)-2-amino-3-methyl-pentanoyl]-4-(trifluoromethyl)pyrrolidine-2-carboxylate; 2,2,2-trifluoroacetaldehyde (Intermediate 38) as yellow oil.

MS obsd. (ESI⁺) [(M+H)⁺]: 387.1.

### Intermediate 39a and 39b

### Tert-butyl N-amino-N-[[(3S)-2-oxo-3-piperidyl]methyl]carbamate and tert-butyl N-amino-N-[[(3R)-2-oxo-3-piperidyl]methyl]carbamate

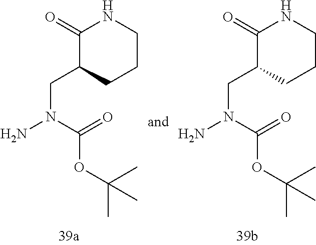 [SMILES: CC(C)(C)OC(=O)N(N)C[C@@H]1CCCNC1=O ; CC(C)(C)OC(=O)N(N)C[C@H]1CCCNC1=O]

### Step 1: Preparation of tert-butyl 3-methylene-2-oxo-piperidine-1-carboxylate

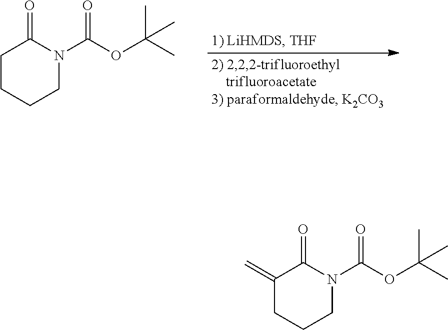

To a solution of 1-Boc-2-piperidone (45.0 g, 225.85 mmol, Bidepharm, CAS number: 85908-96-9) in THF (500 mL) was added LiHMDS (474.3 mL, 474.3 mmol) at 0° C. under N₂ atmosphere. The reaction mixture was stirred at 20° C. for 30 min and then cooled to 0° C. To the mixture was added 2,2,2-trifluoroethyl trifluoroacetate (62.75 g, 320.06 mmol) at 0° C. Then the mixture was stirred at 20° C. for 50 min. The reaction was quenched with saturated NH₄Cl (500 mL) and extracted with EtOAc (300 mL×2). The organic layers were washed with brine (100 mL×3), dried over Na₂SO₄, filtered and concentrated in vacuum. The residue was dissolved in toluene (500 mL). To the solution was added paraformaldehyde (20.3 g, 677.54 mmol) and K₂CO₃ (68.7 g, 496.86 mmol). The reaction mixture was stirred at 110° C. for 2 h under N₂ atmosphere. The reaction was diluted with EtOAc (250 mL) and water (250 mL). The mixture was extracted with EtOAc (200 mL×2). The organic layers were washed with brine (150 mL×3), dried over Na₂SO₄, filtered and concentrated in vacuum to afford tert-butyl 3-methylene-2-oxo-piperidine-1-carboxylate (26.5 g, crude) as yellow oil.

MS obsd. (ESI⁺) [(M-tBu+H)⁺]: 156.0.

### Step 2: Preparation of tert-butyl 3-[(2-benzyloxycarbonylhydrazino)methyl]-2-oxo-piperidine-1-carboxylate

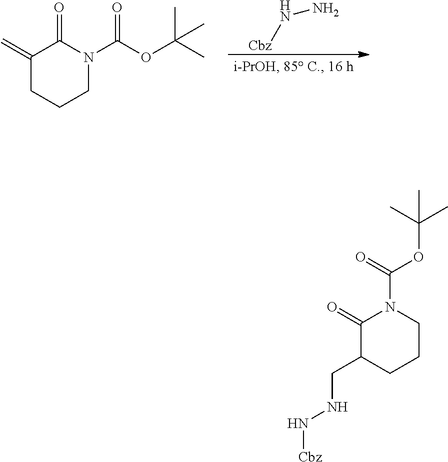 [SMILES: C=C1CCCN(C(=O)OC(C)(C)C)C1=O ; NNC(=O)OCc1ccccc1 ; CC(C)(C)OC(=O)N1CCCC(CNNC(=O)OCc2ccccc2)C1=O]

To a solution of tert-butyl 3-methylene-2-oxo-piperidine-1-carboxylate (26.5 g, 125.4 mmol) in i-PrOH (300 mL) was added benzyl carbazate (20.85 g, 125.44 mmol). The mixture was degassed with N₂ for 3 times. The resulting mixture was stirred at 85° C. for 12 h. The mixture was concentrated in vacuum to afford tert-butyl 3-[(2-benzyloxycarbonylhydrazino)methyl]-2-oxo-piperidine-1-carboxylate (47.3 g, crude) as yellow oil.

MS obsd. (ESI⁺) [(M-Boc+H)⁺]: 278.4.

### Step 3: Preparation of benzyl N-[(2-oxo-3-piperidyl)methylamino]carbamate; 2,2,2-trifluoroacetic acid

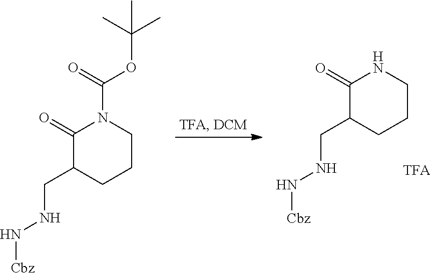 [SMILES: CC(C)(C)OC(=O)N1CCCC(CNNC(=O)OCc2ccccc2)C1=O ; O=C(NNCC1CCCNC1=O)OCc1ccccc1]

To a solution of tert-butyl 3-[(2-benzyloxycarbonylhydrazino)methyl]-2-oxo-piperidine-1-carboxylate (47.3 g, 125.3 mmol) in DCM (320 mL) was added TFA (250.0 mL). The mixture was stirred at 20° C. for 30 min and concentrated in vacuum to afford benzyl N-[(2-oxo-3-piperidyl)methylamino]carbamate; 2,2,2-trifluoroacetic acid (49.0 g, crude) as yellow oil.

MS obsd. (ESI⁺) [(M+H)⁺]: 278.1.

### Step 4: Preparation of tert-butyl N-(benzyloxycarbonylamino)-N-[(2-oxo-3-piperidyl)methyl]carbamate

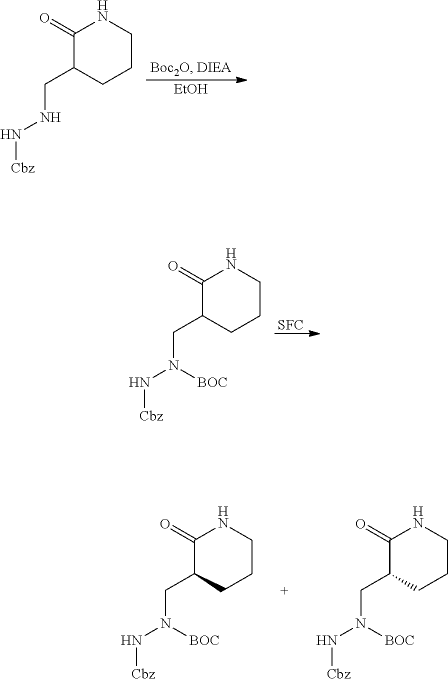

To a solution of benzyl N-[(2-oxo-3-piperidyl)methylamino]carbamate; 2,2,2-trifluoroacetic acid (49.0 g, 125.2 mmol) in methanol (500 mL) was added DIPEA (80.9 g, 626.1 mmol) and Boc₂O (32.9 g, 162.8 mmol) at 0° C. Then the mixture was stirred at 50° C. for 12 h and at 80° C. for another 12 h. The resulting mixture was concentrated in vacuum and the residue was purified by reverse flash and silica gel column to afford tert-butyl N-(benzyloxycarbonylamino)-N-[(2-oxo-3-piperidyl)methyl]carbamate (3.07 g) as a light yellow solid. 10 g of the racemic product was split by prep-SFC (Sample preparation: Add MeOH 400 mL into sample; Instrument: Thr 80; Mobile Phase: 45% MeOH (NEU) in Supercritical CO₂; Flow Rate: 70 g/min; Cycle Time: 4.62 min, total time: 600 min; Single injection volume: 3.5 mL; Back Pressure: 100 bar to keep the CO₂ in Supercritical flow) afford tert-butyl N-(benzyloxycarbonylamino)-N-[[(3R)-2-oxo-3-piperidyl]methyl]carbamate (retention time: 3.49 min) (4.1 g) and tert-butyl N-(benzyloxycarbonylamino)-N-[[(3S)-2-oxo-3-piperidyl]methyl]carbamate (retention time: 4.83 min) (4.2 g) as yellow oil.

MS obsd. (ESI⁺) [(M+H)⁺]: 378.2.

### Step 5: Preparation of tert-butyl N-amino-N-[(2-oxo-3-piperidyl)methyl]carbamate

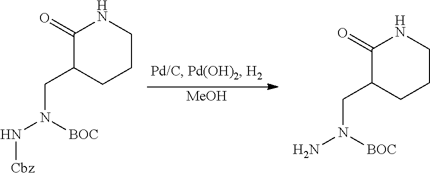 [SMILES: "MeOH" ; CC(C)(C)OC(=O)N(CC1CCCNC1=O)NC(=O)OCc1ccccc1 ; CC(C)(C)OC(=O)N(N)CC1CCCNC1=O]

To a solution of tert-butyl N-(benzyloxycarbonylamino)-N-[(2-oxo-3-piperidyl)methyl]carbamate (1.0 g, 2.65 mmol) in methanol (20 mL) was added Pd(OH)₂ (50 mg) and Pd/C (50 mg, 10% purity) under N₂ atmosphere. The suspension was degassed with H₂ for 3 times. The resulting mixture was stirred at 25° C. for 12 h under a H₂ balloon. The suspension was filtered and the filtrate was concentrated in vacuum to afford the racemic tert-butyl N-amino-N-[(2-oxo-3-piperidyl)methyl]carbamate (570 mg, racemic) as light yellow oil.

MS obsd. (ESI⁺) [(M-Boc+H)⁺]: 144.2.

### Step 6: Preparation of tert-butyl N-amino-N-[[(3S)-2-oxo-3-piperidyl]methyl]carbamate

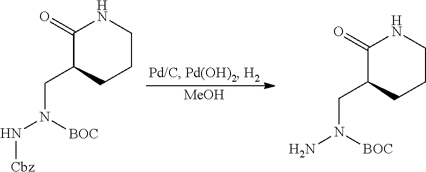 [SMILES: "MeOH" ; CC(C)(C)OC(=O)N(C[C@@H]1CCCNC1=O)NC(=O)OCc1ccccc1 ; CC(C)(C)OC(=O)N(N)C[C@@H]1CCCNC1=O]

To a solution of tert-butyl N-(benzyloxycarbonylamino)-N-[[(3S)-2-oxo-3-piperidyl]methyl]carbamate (3.6 g, 9.54 mmol) in methanol (40 mL) was added Pd(OH)₂ (200 mg) and Pd/C (200.0 mg, 10% purity). The mixture was degassed with H₂ for 3 times and stirred at 25° C. for 2 h under H₂ balloon. The reaction mixture was filtered and the filtrate was concentrated in vacuum to afford tert-butyl N-amino-N-[[(3S)-2-oxo-3-piperidyl]methyl]carbamate (2.3 g, Intermediate 39a) as colorless oil.

MS obsd. (ESI⁺) [(M+H)⁺]: 244.4.

### Step 7: Preparation of tert-butyl N-amino-N-[[(3R)-2-oxo-3-piperidyl]methyl]carbamate

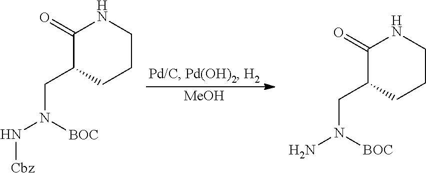 [SMILES: "MeOH" ; CC(C)(C)OC(=O)N(C[C@H]1CCCNC1=O)NC(=O)OCc1ccccc1 ; CC(C)(C)OC(=O)N(N)C[C@H]1CCCNC1=O]

To a solution of tert-butyl N-(benzyloxycarbonylamino)-N-[[(3S)-2-oxo-3-piperidyl]methyl]carbamate (3.6 g, 9.54 mmol) in methanol (20 mL) was added Pd(OH)₂ (360 mg) and Pd/C (360 mg, 10% purity). The mixture was degassed with H₂ for 3 times and stirred at 25° C. for 2 h under H₂ balloon. The reaction mixture was filtered and the filtrate was concentrated in vacuum to afford tert-butyl N-amino-N-[[(3S)-2-oxo-3-piperidyl]methyl]carbamate (2.3 g, Intermediate 39b) as colorless oil.

MS obsd. (ESI+) [(M-Boc+H)+]: 144.2.

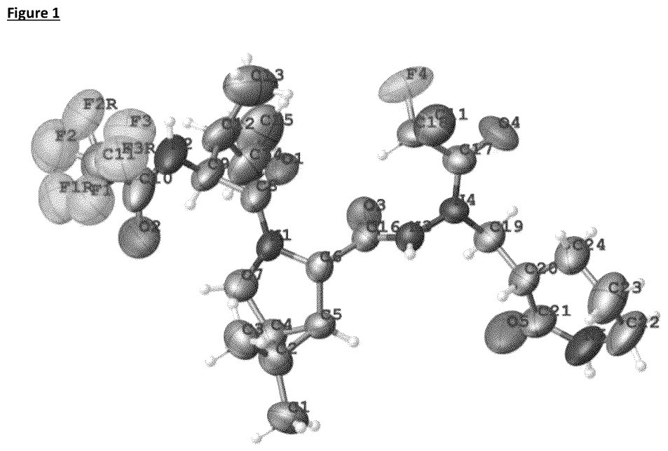

Intermediate 39a was used in the preparation of N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide (Example 165a), which was crystallized and analyzed by single crystal X-ray diffraction (FIG. 1). This confirmed the absolute configuration of Intermediate 39a being (S)-configuration and corresponding pressure tert-butyl N-(benzyloxycarbonylamino)-N-[[(3S)-2-oxo-3-piperidyl]methyl]carbamate. The enantiomer of 39a was designated to be (R)-configuration (39b).

The procedure of preparation of N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide was as follows.

### Step 1: Preparation of methyl (1R,2S,5S)-3-[(2S,3S)-2-(tert-butoxycarbonylamino)-3-methyl-pentanoyl]-6,6-dimethyl-3-azabicyclo[3.1.0]hexane-2-carboxylate

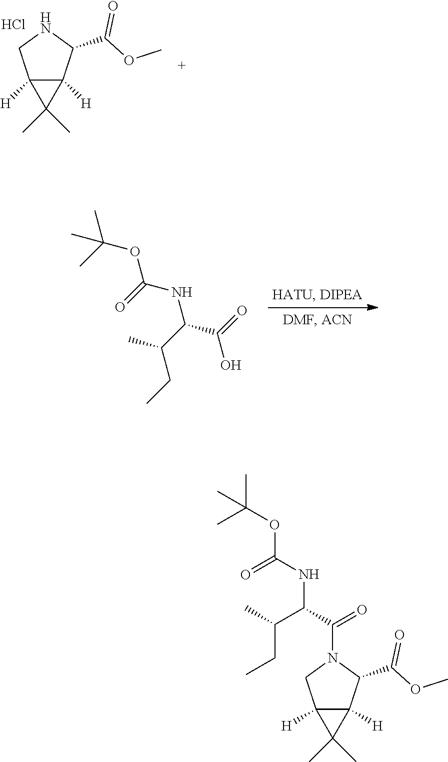 [SMILES: COC(=O)[C@H]1NC[C@H]2[C@@H]1C2(C)C ; Cl ; CC[C@H](C)[C@H](NC(=O)OC(C)(C)C)C(=O)O ; CC[C@H](C)[C@H](NC(=O)OC(C)(C)C)C(=O)N1C[C@H]2[C@@H]([C@H]1C(=O)OC)C2(C)C]

To a solution of Boc-Ile-OH (96.0 g, 415.06 mmol) in DMF (192 mL) and MeCN (1536 mL) were added methyl (1R,2S,5S)-6,6-dimethyl-3-azabicyclo[3.1.0]hexane-2-carboxylate; hydrochloride (89.64 g, 435.82 mmol) and HATU (173.6 g, 456.57 mmol) at 0° C. under N₂ to give a colorless solution, followed by drop-wise addition of DIPEA (216.88 mL, 1245.19 mmol) at 0° C. under N₂ to give a light yellow solution. The reaction was then allowed to warm to 20° C. and was stirred for 16 h under N₂ atmosphere and concentrated in vacuum. The residue was added to ethyl acetate (2400 mL) and water (3600 mL). The resulting suspension was filtered and the filter cake was washed with ethyl acetate (500 mL), and the filter cake was dried in vacuum to give methyl (1R,2S,5S)-3-[(2S,3S)-2-(tert-butoxycarbonylamino)-3-methyl-pentanoyl]-6,6-dimethyl-3-azabicyclo[3.1.0]hexane-2-carboxylate (75.0 g) as a white solid.

MS obsd. (ESI⁺) [(M+H)⁺]: 383.2.

### Step 2: Preparation of (1R,2S,5S)-3-[(2S,3S)-2-(tert-butoxycarbonylamino)-3-methyl-pentanoyl]-6,6-dimethyl-3-azabicyclo[3.1.0]hexane-2-carboxylic acid

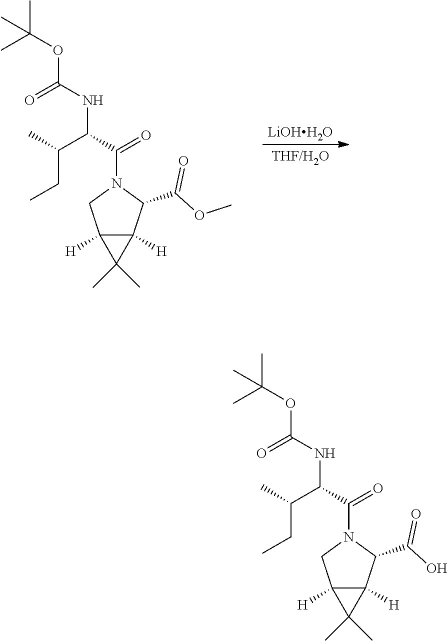 [SMILES: CC[C@H](C)[C@H](NC(=O)OC(C)(C)C)C(=O)N1C[C@H]2[C@@H]([C@H]1C(=O)OC)C2(C)C ; "LiOH·H2O" ; CC[C@H](C)[C@H](NC(=O)OC(C)(C)C)C(=O)N1C[C@H]2[C@@H]([C@H]1C(=O)O)C2(C)C]

To a solution of methyl (1R,2S,5S)-3-[(2S,3S)-2-(tert-butoxycarbonylamino)-3-methyl-pentanoyl]-6,6-dimethyl-3-azabicyclo[3.1.0]hexane-2-carboxylate (50.0 g, 130.72 mmol) in THF (500 mL). The mixture was added a solution of LiOH·H₂O (10.97 g, 261.4 mmol) in water (500 mL). Then the mixture was stirred at 25° C. for 2 h. The reaction mixture was poured into ice/water (300 mL) and acidified with 12N HCl to pH=5. Then the mixture was extracted with EA (500 ml×2). The combined organic phases were washed with brine (100 mL), dried over Na₂SO₄ and concentrated in vacuum to give (1R,2S,5S)-3-[(2S,3S)-2-(tert-butoxycarbonylamino)-3-methyl-pentanoyl]-6,6-dimethyl-3-azabicyclo[3.1.0]hexane-2-carboxylic acid (48.1 g) as yellow oil.

MS obsd. (ESI⁺) [(M+H)⁺]: 369.2.

### Step 3: Preparation of (1R,2S,5S)-3-[(2S,3S)-2-amino-3-methyl-pentanoyl]-6,6-dimethyl-3-azabicyclo[3.1.0]hexane-2-carboxylic acid; hydrochloride

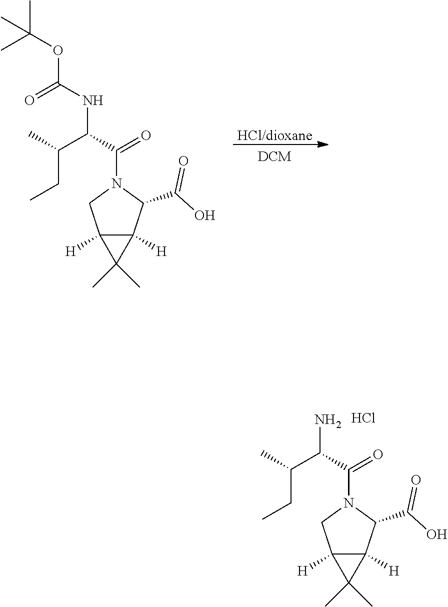 [SMILES: CC[C@H](C)[C@H](NC(=O)OC(C)(C)C)C(=O)N1C[C@H]2[C@@H]([C@H]1C(=O)O)C2(C)C ; "HCl/dioxane" ; Cl ; CC[C@H](C)[C@H](N)C(=O)N1C[C@H]2[C@@H]([C@H]1C(=O)O)C2(C)C]

To a solution of (1R,2S,5S)-3-[(2S,3S)-2-(tert-butoxycarbonylamino)-3-methyl-pentanoyl]-6,6-dimethyl-3-azabicyclo[3.1.0]hexane-2-carboxylic acid (48.1 g, 130.54 mmol) in DCM (500 mL) was added HCl/dioxane (500.0 mL, 2000.0 mmol). The mixture was stirred at 25° C. for 2 h and concentrated in vacuum to afford (1R,2S,5S)-3-[(2S,3S)-2-amino-3-methyl-pentanoyl]-6,6-dimethyl-3-azabicyclo[3.1.0]hexane-2-carboxylic acid; hydrochloride (39.8 g) as a yellow foam.

### Step 4: Preparation of (1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carboxylic acid

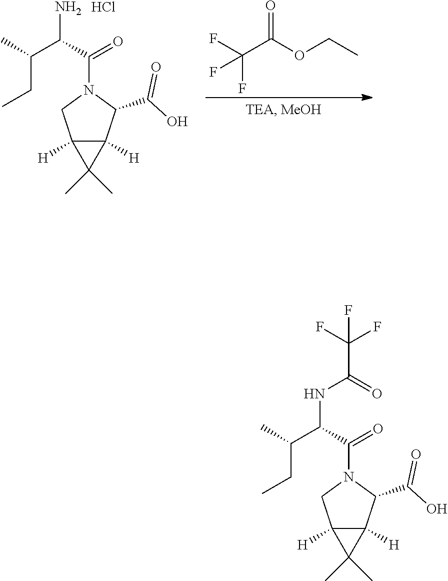

To a solution of (1R,2S,5S)-3-[(2S,3R)-2-amino-3-methyl-pentanoyl]-6,6-dimethyl-3-azabicyclo[3.1.0]hexane-2-carboxylic acid; hydrochloride (39.79 g, 130.54 mmol) in methanol (400 mL) was added TEA (79.26 g, 783.24 mmol) dropwise at 0° C., followed by ethyl trifluoroacetate (55.64 g, 391.62 mmol). Then the mixture was warmed to 50° C. and stirred at 50° C. for 12 h. The mixture was diluted in EtOAc (500 mL), washed with brine (300 ml), dried over Na₂SO₄ and concentrated to give (1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carboxylic acid (45.0 g) as a yellow foam.

MS obsd. (ESI⁺) [(M+H)⁺]: 365.1.

### Step 5: Preparation of tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3S)-2-oxo-3-piperidyl]methyl]carbamate

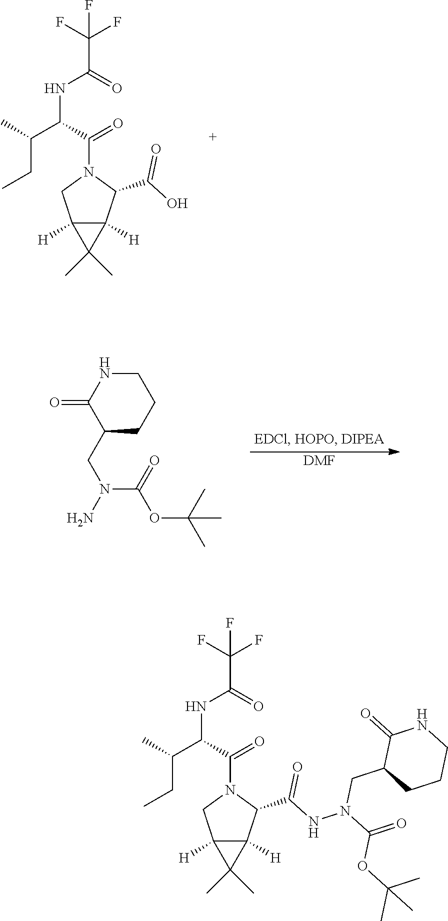

To a solution of (1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carboxylic acid (150 mg, 0.41 mmol) in DMF (5 mL) was added DIPEA (266 mg, 2.06 mmol). After cooling to 0° C., to the mixture was added HOPO (55 mg, 0.49 mmol), EDCI (157 mg, 0.49 mmol) and tert-butyl N-amino-N-[[(3S)-2-oxo-3-piperidyl]methyl]carbamate (100 mg, 0.41 mmol). Then the mixture was stirred at 30° C. for 12 h. The resulting mixture was poured into water (30 mL) at 25° C. with stirring, extracted with EtOAc (50 mL×2). The combined organic phase was washed brine (40 mL×2), followed by 1N HCl (50 mL), 10% aqueous K₂CO₃ solution (50 mL) and brined (30 mL), dried over Na₂SO₄ and concentrated in vacuum to afford tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3S)-2-oxo-3-piperidyl]methyl]carbamate (242 mg) as a white solid.

MS obsd. (ESI⁺) [(M+H)⁺]: 590.5.

### Step 6: Preparation of N-[(1S,2S)-1-[(1R,2S,5S)-6,6-dimethyl-2-[[[(3S)-2-oxo-3-piperidyl]methylamino]carbamoyl]-3-azabicyclo[3.1.0]hexane-3-carbonyl]-2-methyl-butyl]-2,2,2-trifluoro-acetamide

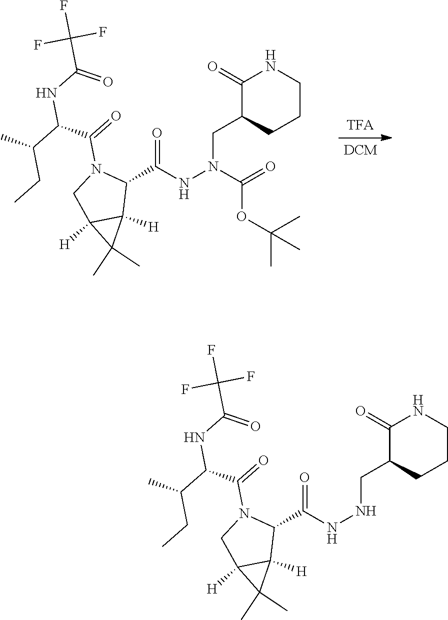 [SMILES: CC[C@H](C)[C@H](NC(=O)C(F)(F)F)C(=O)N1C[C@H]2[C@@H]([C@H]1C(=O)NN(C[C@@H]1CCCNC1=O)C(=O)OC(C)(C)C)C2(C)C ; CC[C@H](C)[C@H](NC(=O)C(F)(F)F)C(=O)N1C[C@H]2[C@@H]([C@H]1C(=O)NNC[C@@H]1CCCNC1=O)C2(C)C]

To a solution of tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3S)-2-oxo-3-piperidyl]methyl]carbamate (242 mg, 0.41 mmol) in DCM (6 mL) was added TFA (3.0 mL). The mixture was stirred at 25° C. for 1 h. The resulting mixture was concentrated in vacuum and the residue was purified by reverse flash (120 g Flash Column; Welch Ultimate XB_C18 20-40 μm; 35 min; 75 mL/min, ACN-Water, 0.1% HCl to afford N-[(1S,2S)-1-[(1R,2S,5S)-6,6-dimethyl-2-[[[(3S)-2-oxo-3-piperidyl]methylamino]carbamoyl]-3-azabicyclo[3.1.0]hexane-3-carbonyl]-2-methyl-butyl]-2,2,2-trifluoro-acetamide; hydrochloride (182 mg) as a white solid.

MS obsd. (ESI⁺) [(M+H)⁺]: 490.2.

### Step 7: Preparation of N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide

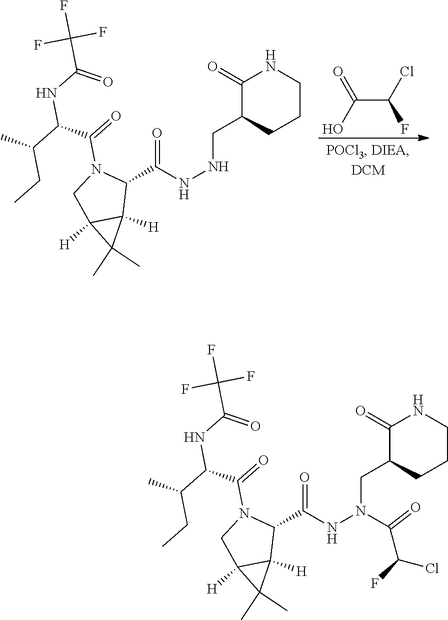

To a solution of (2R)-2-chloro-2-fluoro-acetic acid (126 mg, 0.69 mmol) in DCM (2 mL) was added POCl₃ (95 mg, 0.62 mmol). The solution was stirred at 25° C. for 1 h. The resulting solution was added dropwise to a solution of N-[(1S,2S)-1-[(1R,2S,5S)-6,6-dimethyl-2-[[[(3S)-2-oxo-3-piperidyl]methylamino]carbamoyl]-3-azabicyclo[3.1.0]hexane-3-carbonyl]-2-methyl-butyl]-2,2,2-trifluoro-acetamide; hydrochloride (182 mg, 0.35 mmol) and DIPEA (447 mg, 3.46 mmol) in DCM (2 mL) at −5° C. The mixture was stirred at the same temperature for 1 h. The reaction was quenched with methanol (2 mL) and 4N HCl/dioxane (2 mL) at −5° C. The resulting mixture was concentrated in vacuum at room temperature. The residue was purified by reverse flash (120 g Flash Column; Welch Ultimate XB_C18 20-40 μm; 35 min; 75 mL/min, ACN-Water, 0.1% FA) to afford N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide (120 mg) as a white solid.

2 mg of N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide was crystalized in a mixed solvent of MeOH and water (0.2 ml, v/v=4/1) at room temperature to afford a transparence crystal.

¹H NMR (400 MHz, DMSO-d₆) δ ppm 11.16 (s, 1H), 9.97 (d, J=7.6 Hz, 1H), 7.57 (s, 1H), 6.90-6.70 (m, 1H), 4.21 (dd, J=10.4 Hz, 7.6 Hz, 1H), 4.15 (s, 1H), 4.00 (dd, J=14.0 Hz, 9.6 Hz, 1H), 3.94-3.83 (m, 2H), 3.46 (dd, J=13.6 Hz, 4.0 Hz, 1H), 3.15-3.07 (m, 2H), 2.48-2.42 (m, 1H), 1.99-1.88 (m, 1H), 1.87-1.73 (m, 2H), 1.69-1.65 (m, 1H), 1.63-1.54 (m, 1H), 1.53-1.40 (m, 3H), 1.17-1.08 (m, 1H), 1.06 (s, 3H), 0.92 (s, 3H), 0.87 (d, J=6.8 Hz, 3H), 0.82 (d, J=7.2 Hz, 3H).

MS obsd. (ESI⁺) [(M+H)⁺]: 584.2.

### Intermediate 40

### Benzyl (1R,2S,5S)-3-[(2S)-2-amino-3,3-dimethyl-pentanoyl]-6,6-dimethyl-3-azabicyclo[3.1.0]hexane-2-carboxylate; 2,2,2-trifluoroacetic acid

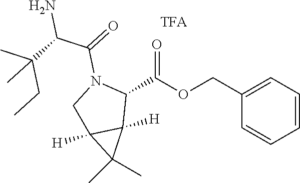 [SMILES: CCC(C)(C)[C@H](N)C(=O)N1C[C@H]2[C@@H]([C@H]1C(=O)OCc1ccccc1)C2(C)C]

The title compound was prepared in analogy to the procedure described for the preparation of Intermediate 1, by using (2S)-2-(tert-butoxycarbonylamino)-3,3-dimethyl-pentanoic acid (Intermediate 25) instead of (2S)-2-(tert-butoxycarbonylamino)-3,3-dimethyl-butanoic acid to afford benzyl (1R,2S,5S)-3-[(2S)-2-amino-3,3-dimethyl-pentanoyl]-6,6-dimethyl-3-azabicyclo[3.1.0]hexane-2-carboxylate; 2,2,2-trifluoroacetic acid (Intermediate 40) as yellow oil.

### Intermediate 41

### Benzyl (3S,3aS,6aR)-2-[(2S,3S)-2-amino-3-methyl-pentanoyl]-3,3a,4,5,6,6a-hexahydro-1H-cyclopenta[c]pyrrole-3-carboxylate; hydrochloride

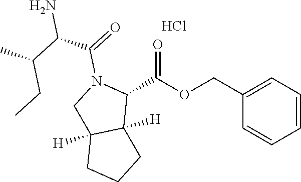 [SMILES: CC[C@H](C)[C@H](N)C(=O)N1C[C@@H]2CCC[C@@H]2[C@H]1C(=O)OCc1ccccc1 ; Cl]

The title compound was prepared in analogy to the procedure described for the preparation of Intermediate 1, by using (3S,3aS,6aR)-2-tert-butoxycarbonyl-3,3a,4,5,6,6a-hexahydro-1H-cyclopenta[c]pyrrole-3-carboxylic acid instead of (1R,2S,5S)-3-tert-butoxycarbonyl-6,6-dimethyl-3-azabicyclo[3.1.0]hexane-2-carboxylic acid and Boc-Ile-OH instead of (2S)-2-(tert-butoxycarbonylamino)-3,3-dimethyl-butanoic acid to afford benzyl (3S,3aS,6aR)-2-[(2S,3S)-2-amino-3-methyl-pentanoyl]-3,3a,4,5,6,6a-hexahydro-1H-cyclopenta[c]pyrrole-3-carboxylate; hydrochloride (Intermediate 41) as yellow oil.

MS obsd. (ESI⁺) [(M+H)⁺]: 359.3.

### Intermediate 42

### Benzyl (1R,2S,5S)-3-[(2S)-2-amino-4,4-dimethyl-pentanoyl]-6,6-dimethyl-3-azabicyclo[3.1.0]hexane-2-carboxylate; 2,2,2-trifluoroacetic acid

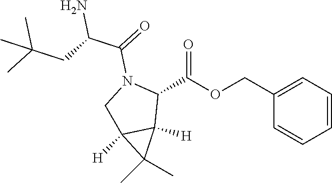 [SMILES: CC(C)(C)C[C@H](N)C(=O)N1C[C@H]2[C@@H]([C@H]1C(=O)OCc1ccccc1)C2(C)C]

The title compound was prepared in analogy to the procedure described for the preparation of Intermediate 1, by (2S)-2-(benzyloxycarbonylamino)-4,4-dimethyl-pentanoic acid (Intermediate 20) instead of (2S)-2-(tert-butoxycarbonylamino)-3,3-dimethyl-butanoic acid to afford benzyl (1R,2S,5S)-3-[(2S)-2-amino-4,4-dimethyl-pentanoyl]-6,6-dimethyl-3-azabicyclo[3.1.0]hexane-2-carboxylate; 2,2,2-trifluoroacetic acid (Intermediate 43) as colorless oil.

MS obsd. (ESI⁺) [(M+H)⁺]: 373.3.

### Intermediate 43

### (2S)-4,4-dimethylpyrrolidine-2-carboxylate; 2,2,2-trifluoroacetic acid

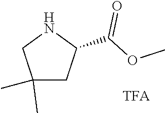

### Step 1: Preparation of O1-tert-butyl O2-methyl (2S)-4,4-dimethylpyrrolidine-1,2-dicarboxylate

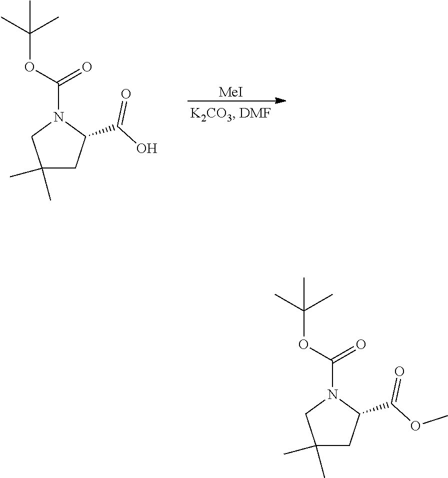 [SMILES: "MeI" ; CC1(C)C[C@@H](C(=O)O)N(C(=O)OC(C)(C)C)C1 ; COC(=O)[C@@H]1CC(C)(C)CN1C(=O)OC(C)(C)C]

To a solution of (2S)-1-tert-butoxycarbonyl-4,4-dimethyl-pyrrolidine-2-carboxylic acid (500 mg, 2.06 mmol) in DMF (10 mL) was added K₂CO₃ (852.07 mg, 6.17 mmol) and Mel (875 mg, 6.17 mmol). The mixture was stirred at 25° C. for 12 h. The mixture was diluted with water (40 mL) and acidified with 1 N HCl to pH=4. The resulting mixture was extracted with EtOAc (50 mL×2). The combined organic phase was washed with brine (60 mL), dried over Na₂SO₄ and concentrated in vacuum to afford 01-tert-butyl 02-methyl (2S)-4,4-dimethylpyrrolidine-1,2-dicarboxylate (528 mg) as colorless oil.

### Step 2: Preparation of (2S)-4,4-dimethylpyrrolidine-2-carboxylate; 2,2,2-trifluoroacetic acid

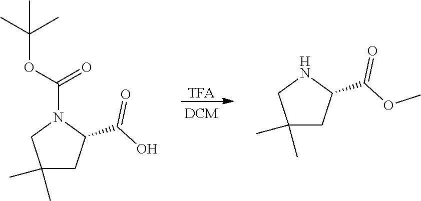

To a solution of 01-tert-butyl 02-methyl (2S)-4,4-dimethylpyrrolidine-1,2-dicarboxylate (528 mg, 2.05 mmol) in DCM (5 mL) was added TFA (2.5 mL). The mixture was stirred at 25° C. for 2 h. The mixture was concentrated in vacuum to afford methyl (2S)-4,4-dimethylpyrrolidine-2-carboxylate; 2,2,2-trifluoroacetic acid (556 mg,) as yellow oil.

### Example 1

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide

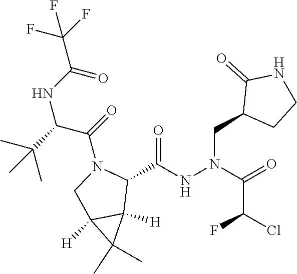 [SMILES: CC(C)(C)[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

### Step 1: Preparation of benzyl (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylate

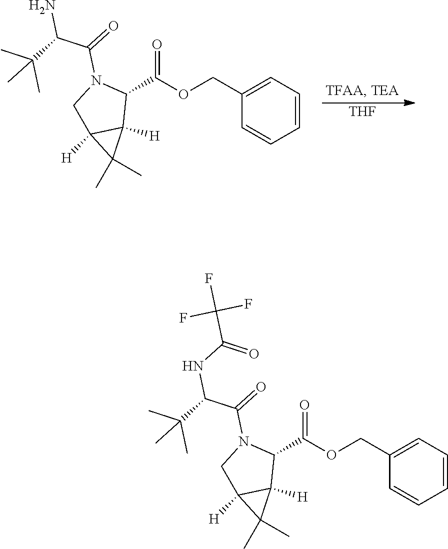 [SMILES: CC(C)(C)[C@H](N)C(=O)N1C[C@H]2[C@@H]([C@H]1C(=O)OCc1ccccc1)C2(C)C ; "THF" ; CC(C)(C)[C@H](NC(=O)C(F)(F)F)C(=O)N1C[C@H]2[C@@H]([C@H]1C(=O)OCc1ccccc1)C2(C)C]

To a solution of benzyl (1R,2S,5S)-3-[(2S)-2-amino-3,3-dimethyl-butanoyl]-6,6-dimethyl-3-azabicyclo[3.1.0]hexane-2-carboxylate (970 mg, 2.71 mmol, Intermediate 1) in THF (20 mL) was added TEA (601 mg, 5.95 mmol) and TFAA (1131 mg, 5.41 mmol) at 0° C. The reaction mixture was stirred at 25° C. for 1 h. The reaction was quenched with water (50 mL) and extracted with EtOAc (80 mL×2). The organic layers were washed with brine (50 mL), dried over anhydrous Na₂SO₄, filtered and concentrated in vacuum. The residue was purified by silica gel column eluted with PE to EtOAc/PE=10/1 to afford benzyl (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylate (1.2 g) as colorless oil.

MS obsd. (ESI⁺) [M+H]⁺. 455.3.

### Step 2: Preparation of (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid

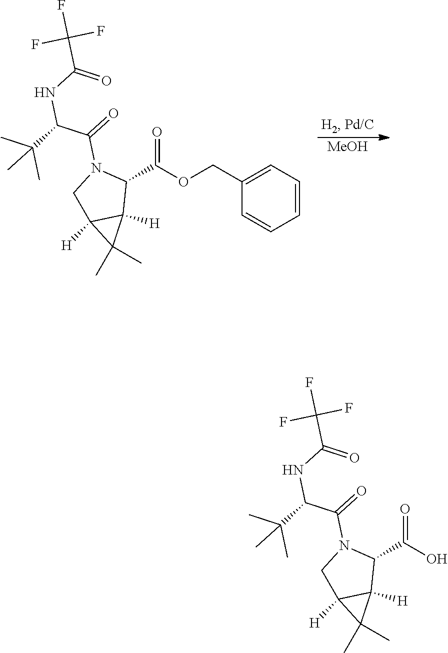 [SMILES: "MeOH" ; CC(C)(C)[C@H](NC(=O)C(F)(F)F)C(=O)N1C[C@H]2[C@@H]([C@H]1C(=O)OCc1ccccc1)C2(C)C ; CC(C)(C)[C@H](NC(=O)C(F)(F)F)C(=O)N1C[C@H]2[C@@H]([C@H]1C(=O)O)C2(C)C]

To a solution of benzyl (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylate (1.2 g, 2.64 mmol) in MeOH (20 mL) was added Pd/C (200 mg, 10% purity). The mixture was degassed under vacuum and purged H₂ for 3 times. The reaction mixture was stirred at 25° C. for 1 h under H₂ balloon. The mixture was filtered through a celite pad and the pad was washed with MeOH (10 mL×3). The filtrate was concentrated in vacuum to afford (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (960 mg) as a white solid.

MS obsd. (ESI⁺) [M+H]⁺. 365.2.

### Step 3: Preparation of tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxopyrrolidin-3-yl]methyl]carbamate

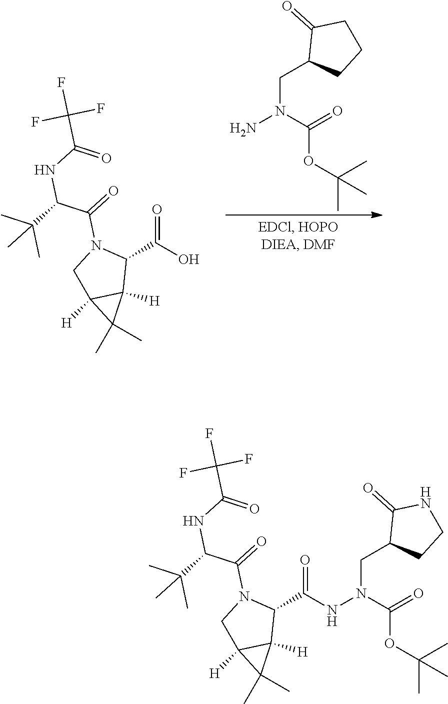 [SMILES: CC(C)(C)[C@H](NC(=O)C(F)(F)F)C(=O)N1C[C@H]2[C@@H]([C@H]1C(=O)O)C2(C)C ; CC(C)(C)OC(=O)N(N)C[C@@H]1CCCC1=O ; CC(C)(C)OC(=O)N(C[C@@H]1CCNC1=O)NC(=O)[C@@H]1[C@@H]2[C@H](CN1C(=O)[C@@H](NC(=O)C(F)(F)F)C(C)(C)C)C2(C)C]

To a solution of (1R,2S,5S)-3-((2S)-3,3-dimethyl-2-(2,2,2-trifluoroacetamido)butanoyl)-6,6-dimethyl-3-azabicyclo[3.1.0]hexane-2-carboxylic acid (10 mg, 0.270 mmol) in DMF (10 mL) was added EDCI (63 mg, 0.330 mmol), HOPO (37 mg, 0.330 mmol), DIPEA (177 mg, 1.37 mmol) and tert-butyl N-amino-N-[[(3S)-2-oxopyrrolidin-3-yl]methyl]carbamate (63 mg, 0.270 mmol, Intermediate 9). The reaction mixture was stirred at 25° C. for 12 h. The mixture was diluted with EtOAc (100 mL), washed with water (30 mL) and brine (30 mL×3). The organic phase was dried over Na₂SO₄, filtered and the filtrate was concentrated. The residue was purified by silica gel column eluted with MeOH/DCM=10/1 to afford tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxopyrrolidin-3-yl]methyl]carbamate (110 mg) as colorless oil.

MS obsd. (ESI⁺) [M+H]⁺: 576.3.

### Step 4: Preparation of N-((1S)-1-((1R,2S,5S)-6,6-dimethyl-2-(2-(((3S)-2-oxopyrrolidin-3-yl)methyl)hydrazine-1-carbonyl)-3-azabicyclo[3.1.0]hexan-3-yl)-3,3-dimethyl-1-oxobutan-2-yl)-2,2,2-trifluoroacetamide

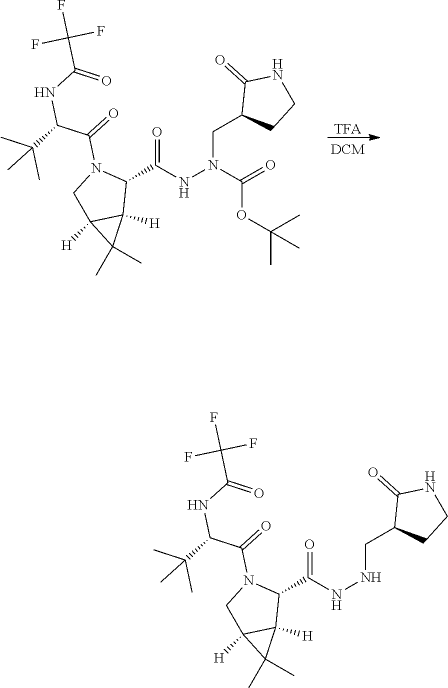

To a solution of tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxopyrrolidin-3-yl]methyl]carbamate (110 mg, 0.190 mmol) in DCM (4 mL) was added TFA (4 mL). The reaction mixture was stirred at 25° C. for 1 h. The mixture was concentrated in vacuum and the residue was purified by prep-HPLC (Column: YMC Triart 30*150 mm*7 um; Condition: ACN in water (0.1% HCl)=31˜51%; Flow Rate (mL/min): 25) to afford N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]-2,2,2-trifluoro-acetamide (80 mg) as a white solid.

MS obsd. (ESI⁺) [M+H]⁺: 476.2.

### Step 5: Preparation of N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide (Example 1)

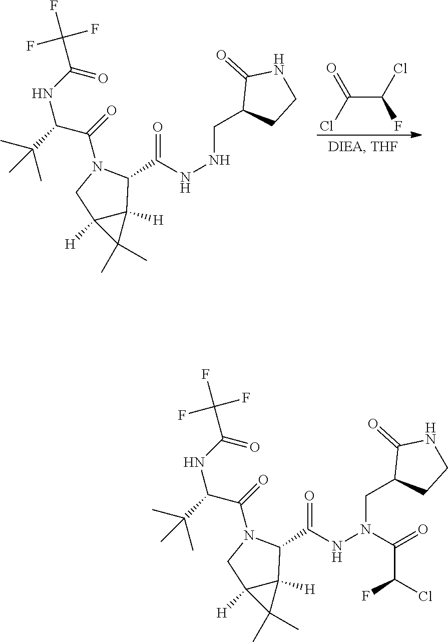

To a solution of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]-2,2,2-trifluoro-acetamide (80 mg, 0.170 mmol) in THF (20 mL) was added DIPEA (260 mg, 2.02 mmol) and (2R)-2-chloro-2-fluoro-acetyl chloride (110 mg, 0.840 mmol, Intermediate 10). The reaction mixture was stirred at 0° C. for 1 h. The mixture was concentrated in vacuum and the residue was purified by prep-HPLC (Column: Phenomenex Synergi Polar-RP 100*25 mm*4 um; Condition: ACN in water (0.1% TFA)=50˜70%; Flow Rate (mL/min): 25) to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide (38.5 mg, Example 1) as a white solid.

¹H NMR (400 MHz, DMSO-d₆) δ: ppm 11.18 (s, 1H), 9.50 (d, J=8.0 Hz, 1H), 7.75 (s, 1H), 6.90 (d, J=50.8 Hz, 1H), 4.40 (d, J=8.0 Hz, 1H), 4.16 (s, 1H), 3.96-3.92 (m, 1H), 3.82-3.69 (m, 2H), 3.47-3.48 (m, 1H), 3.21-3.12 (m, 2H), 2.60-2.56 (m, 1H), 2.20-2.08 (m, 1H), 1.79-1.74 (m, 1H), 1.69-1.59 (m, 1H), 1.54 (d, J=7.6 Hz, 1H), 1.04 (s, 3H), 0.98 (s, 9H), 0.92-0.86 (m, 3H)

MS obsd. (ESI⁺) [M+H]⁺: 570.2.

### Example 2

### N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide

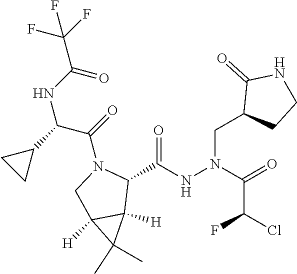 [SMILES: CC1(C)[C@@H]2[C@@H](C(=O)NN(C[C@@H]3CCNC3=O)C(=O)[C@H](F)Cl)N(C(=O)[C@@H](NC(=O)C(F)(F)F)C3CC3)C[C@@H]21]

The title compound was prepared in analogy to the procedure described for the preparation of Example 1, by using benzyl (1R,2S,5S)-3-[(2S)-2-amino-2-cyclopropyl-acetyl]-6,6-dimethyl-3-azabicyclo[3.1.0]hexane-2-carboxylate (Intermediate 2) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide (Example 2) as a white solid.

¹H NMR (400 MHz, CDCl₃) δ: ppm 10.22 (s, 1H), 7.50-7.49 (m, 1H), 6.65-6.52 (m, 1H), 6.22 (s, 1H), 4.59-4.54 (m, 1H), 4.38-4.34 (m, 2H), 4.01-3.97 (m, 1H), 3.82 (d, J=8.8 Hz, 1H), 3.41-3.39 (m, 2H), 2.92-2.81 (m, 2H), 2.42-2.36 (m, 1H), 1.70-1.69 (m, 1H), 1.41-1.39 (m, 1H), 1.20-1.19 (m, 1H), 1.14 (s, 0.5H), 1.07 (s, 3H), 1.00 (s, 0.5H), 0.92 (s, 3H), 0.61-0.59 (m, 2H), 0.55-0.52 (m, 1H), 0.46-0.42 (m, 1H)

MS obsd. (ESI⁺) [(M+H)⁺]: 554.2.

### Example 3

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide

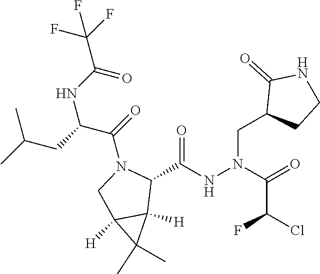 [SMILES: CC(C)C[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

### Step 1: Preparation of methyl (1R,2S,5S)-3-[(2S)-2-(benzyloxycarbonylamino)-4-methyl-pentanoyl]-6,6-dimethyl-3-azabicyclo[3.1.0]hexane-2-carboxylate

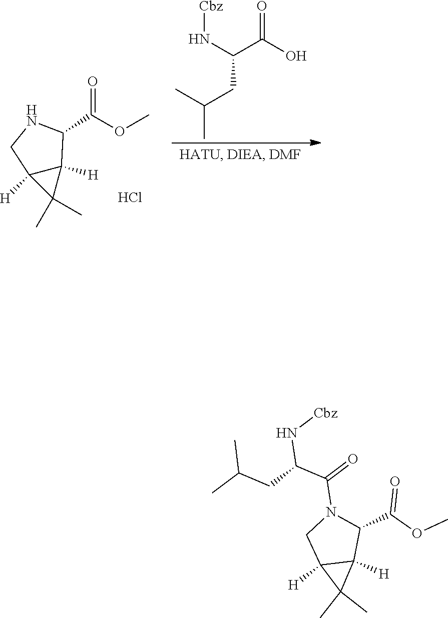

To a solution of (2S)-2-(benzyloxycarbonylamino)-4-methyl-pentanoic acid (322 mg, 1.22 mmol), methyl (1R,2S,5S)-6,6-dimethyl-3-azabicyclo[3.1.0]hexane-2-carboxylate; hydrochloride (250 mg, 1.22 mmol) in DCM (5 mL) was added DIPEA (471 mg, 3.65 mmol) and HATU (343 mg, 1.46 mmol). The mixture was stirred at 25° C. for 12 h. The mixture was concentrated in vacuum and the residue was purified by reverse flash eluted with ACN in H₂O (0.1% NH₃·H₂O)=0˜70% to afford methyl (1R,2S,5S)-3-[(2S)-2-(benzyloxycarbonylamino)-4-methyl-pentanoyl]-6,6-dimethyl-3-azabicyclo[3.1.0]hexane-2-carboxylate (330 mg) as colorless oil

MS obsd. (ESI⁺) [(M+H)⁺]: 417.2.

### Step 2: Preparation of (1R,2S,5S)-3-[(2S)-2-(benzyloxycarbonylamino)-4-methyl-pentanoyl]-6,6-dimethyl-3-azabicyclo[3.1.0]hexane-2-carboxylic acid

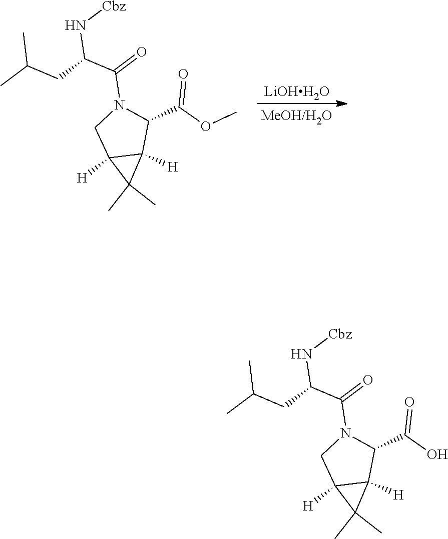

To a solution of methyl (1R,2S,5S)-3-[(2S)-2-(benzyloxycarbonylamino)-4-methyl-pentanoyl]-6,6-dimethyl-3-azabicyclo[3.1.0]hexane-2-carboxylate (330 mg, 0.790 mmol) in THF (3 mL) and water (3 mL) was added LiOH·H₂O (33 mg, 0.790 mmol). The mixture was stirred at 25° C. for 12 h. The reaction mixture was diluted with water 30 mL and extracted with EtOAc (30 mL×3). The combined organic layers were washed with brine 40 mL, dried over Na₂SO₄, filtered and the filtrate was concentrated. The residue was purified by silica gel column eluted with PE/EtOAc=10/1 to 3/1 to afford (1R,2S,5S)-3-[(2S)-2-(benzyloxycarbonylamino)-4-methyl-pentanoyl]-6,6-dimethyl-3-azabicyclo[3.1.0]hexane-2-carboxylic acid (310 mg) as yellow oil.

MS obsd. (ESI⁺) [(M+H)⁺]: 403.1.

### Step 3: Preparation of tert-butyl N-[[(1R,2S,5S)-3-[(2S)-2-(benzyloxycarbonylamino)-4-methyl-pentanoyl]-6,6-dimethyl-3-azabicyclo[3.1.0]hexane-2-carbonyl]amino]-N-[[(3S)-2-oxopyrrolidin-3-yl]methyl]carbamate

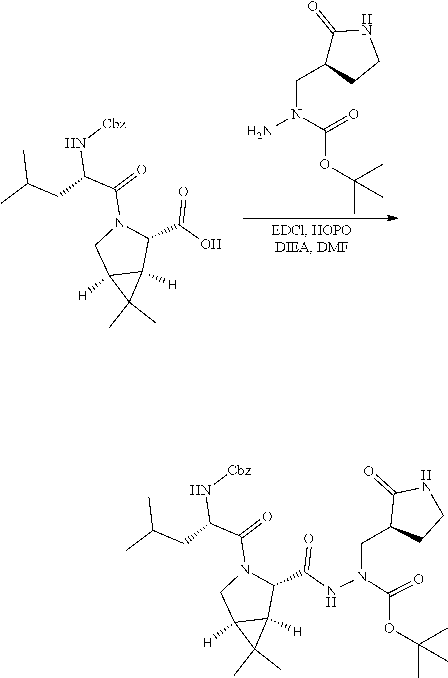 [SMILES: CC(C)C[C@H](NC(=O)OCc1ccccc1)C(=O)N1C[C@H]2[C@@H]([C@H]1C(=O)O)C2(C)C ; CC(C)(C)OC(=O)N(N)C[C@@H]1CCNC1=O ; CC(C)C[C@H](NC(=O)OCc1ccccc1)C(=O)N1C[C@H]2[C@@H]([C@H]1C(=O)NN(C[C@@H]1CCNC1=O)C(=O)OC(C)(C)C)C2(C)C]

To a solution of (1R,2S,5S)-3-[(2S)-2-(benzyloxycarbonylamino)-4-methyl-pentanoyl]-6,6-dimethyl-3-azabicyclo[3.1.0]hexane-2-carboxylic acid (310 mg, 0.770 mmol) in DMF (6 mL) was added DIPEA (497 mg, 3.85 mmol), EDCI (177 mg, 0.920 mmol), HOPO (103 mg, 0.920 mmol) and tert-butyl N-amino-N-[[(3S)-2-oxopyrrolidin-3-yl]methyl]carbamate (194 mg, 0.850 mmol, Intermediate 9). The mixture was stirred at 25° C. for 12 h. The reaction mixture was diluted with water (30 mL) and extracted with EtOAc (30 mL×3). The combined organic layers were washed with brine (40 mL), dried over Na₂SO₄, filtered and concentrated. The residue was purified by silica gel column eluted with PE/EtOAc=10/1 to 3/1 to afford tert-butyl N-[[(1R,2S,5S)-3-[(2S)-2-(benzyloxycarbonylamino)-4-methyl-pentanoyl]-6,6-dimethyl-3-azabicyclo[3.1.0]hexane-2-carbonyl]amino]-N-[[(3S)-2-oxopyrrolidin-3-yl]methyl]carbamate (460 mg) as yellow oil.

MS obsd. (ESI⁺) [(M+H)⁺]: 614.1.

### Step 4: Preparation of tert-butyl N-[[(1R,2S,5S)-3-[(2S)-2-amino-4-methyl-pentanoyl]-6,6-dimethyl-3-azabicyclo[3.1.0]hexane-2-carbonyl]amino]-N-[[(3S)-2-oxopyrrolidin-3-yl]methyl]carbamate

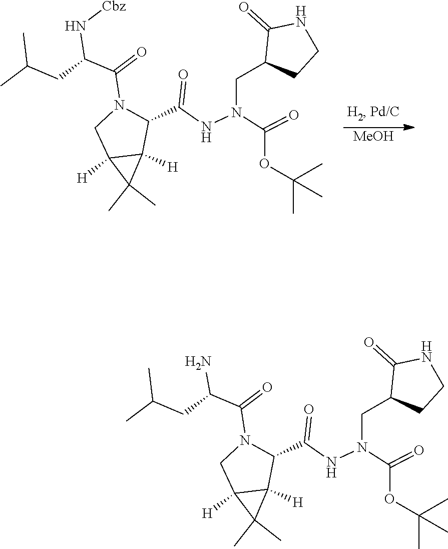

To a solution of tert-butyl N-[[(1R,2S,5S)-3-[(2S)-2-(benzyloxycarbonylamino)-4-methyl-pentanoyl]-6,6-dimethyl-3-azabicyclo[3.1.0]hexane-2-carbonyl]amino]-N-[[(3S)-2-oxopyrrolidin-3-yl]methyl]carbamate (460 mg, 0.750 mmol) in MeOH (6 mL) was added Pd/C (150 mg). The mixture was degassed under vacuum and purged H₂ for 3 times. The resulting mixture was stirred under H₂ at 25° C. for 2 h. The reaction mixture filtered through a celite pad and the pad was washed with MeOH (30 mL). The filtrate was concentrated in vacuum to afford tert-butyl N-[[(1R,2S,5S)-3-[(2S)-2-amino-4-methyl-pentanoyl]-6,6-dimethyl-3-azabicyclo[3.1.0]hexane-2-carbonyl]amino]-N-[[(3S)-2-oxopyrrolidin-3-yl]methyl]carbamate (310 mg) as yellow oil.

MS obsd. (ESI⁺) [(M+H)⁺]: 480.4.

### Step 5: Preparation of tert-butyl N-[[(1R,2S,5S)-6,6-dimethyl-3-[(2S)-4-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate

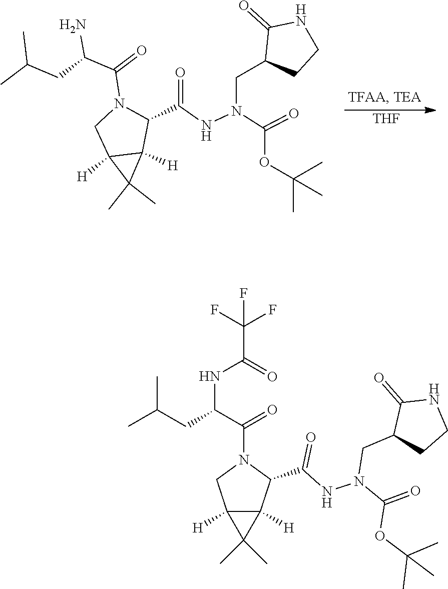

To a solution of tert-butyl N-[[(1R,2S,5S)-3-[(2S)-2-amino-4-methyl-pentanoyl]-6,6-dimethyl-3-azabicyclo[3.1.0]hexane-2-carbonyl]amino]-N-[[(3S)-2-oxopyrrolidin-3-yl]methyl]carbamate (310 mg, 0.65 mmol) in THF (5 mL) was added TEA (163 mg, 1.62 mmol) and TFAA (0.14 mL, 0.970 mmol) at 0° C. The reaction mixture was stirred at 25° C. for 1 h. The mixture was quenched with water (20 mL) and extracted with EtOAc (30 mL×2). The organic layers were washed with brine (50 mL), dried over Na₂SO₄, filtered and the filtrate was concentrated. The residue was purified by silica gel column eluted with PE/EtOAc=20/1 to 5/1 to afford tert-butyl N-[[(1R,2S,5S)-6,6-dimethyl-3-[(2S)-4-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate (310 mg) as yellow oil. MS obsd. (ESI⁺) [(M+H)⁺]: 576.4.

### Step 6: Preparation of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-3-methyl-butyl]-2,2,2-trifluoro-acetamide

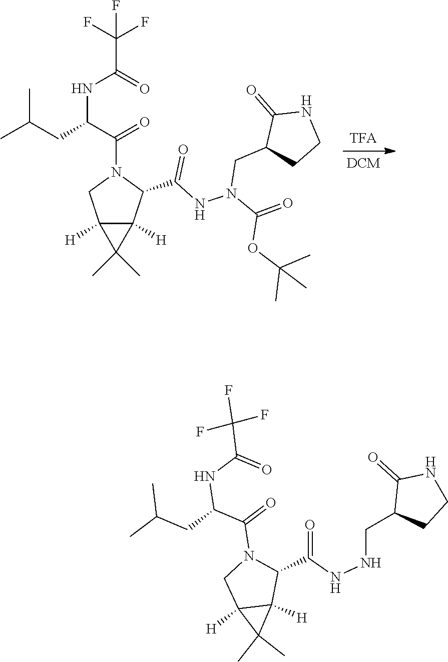 [SMILES: CC(C)C[C@H](NC(=O)C(F)(F)F)C(=O)N1C[C@H]2[C@@H]([C@H]1C(=O)NN(C[C@@H]1CCNC1=O)C(=O)OC(C)(C)C)C2(C)C ; CC(C)C[C@H](NC(=O)C(F)(F)F)C(=O)N1C[C@H]2[C@@H]([C@H]1C(=O)NNC[C@@H]1CCNC1=O)C2(C)C]

To a solution of tert-butyl N-[[(1R,2S,5S)-6,6-dimethyl-3-[(2S)-4-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate (310 mg, 0.54 mmol) in DCM (5 mL) was added TFA (5.0 mL). The mixture was stirred at 25° C. for 1 h. The mixture was concentrated in vacuum. The residue was purified by reversed-flash eluted with ACN in H₂O (0.1% HCl condition)=0˜70% to afford N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-3-methyl-butyl]-2,2,2-trifluoro-acetamide; hydrochloride (170 mg) as yellow oil.

MS obsd. (ESI⁺) [(M+H)⁺]: 476.2.

### Step 7: Preparation of N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide Example 3

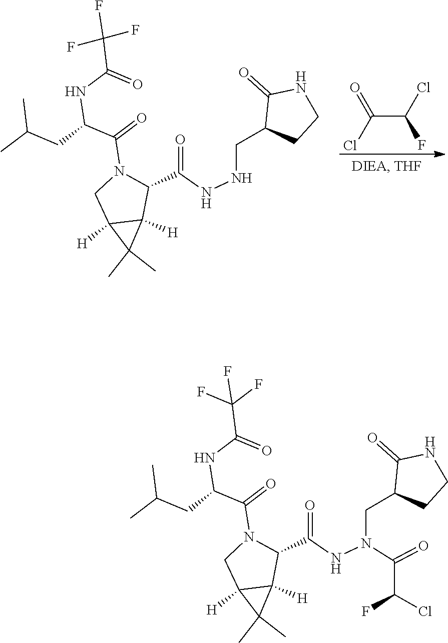

To a solution of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-3-methyl-butyl]-2,2,2-trifluoro-acetamide; hydrochloride (170 mg, 0.36 mmol) in THF (60 mL) was added DIPEA (553 mg, 4.29 mmol) and (2R)-2-chloro-2-fluoro-acetyl chloride (234 mg, 1.79 mmol, Intermediate 10) at 0° C. The reaction mixture was stirred at 0° C. for 1 h. The mixture was concentrated in vacuum. The residue was purified by prep-HPLC (0.1% TFA, Phenomenex luna C18 150*40 mm*15 um; mobile phase: [water (0.1% TFA)-ACN]; B %: 35%-65%, 10 min) to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide (20 mg, Example 3) as an off-white solid.

¹H NMR (400 MHz, CD₃OD) δ: ppm 6.87-6.68 (d, 1H), 4.60 (dd, J=4.8, 9.6 Hz, 1H), 4.28 (s, 1H), 4.06 (dd, J=8.4, 14.0 Hz, 1H), 3.99-3.87 (m, 2H), 3.59 (dd, J=4.8, 14.0 Hz, 1H), 3.43-3.34 (m, 2H), 2.74 (dq, J=4.8, 8.8 Hz, 1H), 2.38-2.25 (m, 1H), 2.10-1.96 (m, 1H), 1.75-1.57 (m, 5H), 1.13 (s, 3H), 1.06 (s, 3H), 0.99 (dd, J=6.0, 11.6 Hz, 6H)

MS obsd. (ESI⁺) [(M+H)⁺]: 570.1.

### Example 4

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide

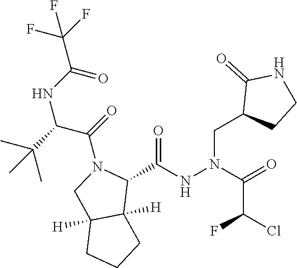 [SMILES: CC(C)(C)[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

### Example 4

The title compound was prepared in analogy to the procedure described for the preparation of Example 1, by using benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate (Intermediate 3) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide (Example 4) as an off-white solid.

¹H NMR (400 MHz, CD₃OD) δ: ppm 7.16-6.66 (m, 1H), 4.72-4.60 (m, 1H), 4.09 (d, J=5.6 Hz, 1H), 4.06-3.95 (m, 2H), 3.78 (dd, J=3.2, 10.4 Hz, 1H), 3.59 (dd, J=4.8, 14.4 Hz, 1H), 3.42-3.33 (m, 2H), 2.94-2.83 (m, 1H), 2.83-2.75 (m, 1H), 2.74 (br. s, 1H), 2.39-2.21 (m, 1H), 2.09-1.70 (m, 6H), 1.52 (m, 1H), 1.08 (s, 9H).

MS obsd. (ESI⁺) [(M+H)⁺]: 570.3.

### Example 5

### N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.2.0]heptane-3-carbonyl]-2,2-dimethyl-propyl]-2,2,2-trifluoro-acetamide

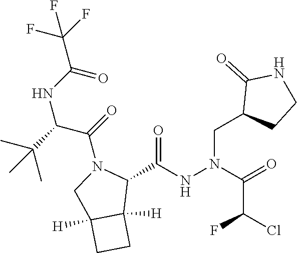 [SMILES: CC(C)(C)[C@H](NC(=O)C(F)(F)F)C(=O)N1C[C@@H]2CC[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (1S,2S,5R)-3-[(2S)-2-amino-3,3-dimethyl-butanoyl]-3-azabicyclo[3.2.0]heptane-2-carboxylate (Intermediate 4) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.2.0]heptane-3-carbonyl]-2,2-dimethyl-propyl]-2,2,2-trifluoro-acetamide (Example 5) as a white solid.

¹H NMR (400 MHz, CDCl₃) δ: ppm 9.83 (br, s, 1H), 7.32-7.28 (m, 1H), 6.62 (d, J=50.4 Hz, 1H), 6.42 (br, s, 1H), 4.88-4.85 (m, 1H), 4.81 (d, J=9.6 Hz, 1H), 4.35 (m, 1H), 3.96-3.93 (m, 2H), 3.45-3.41 (m, 2H), 3.20 (m, 1H), 2.90 (m, 3H), 2.39-2.35 (m, 2H), 1.85-1.81 (m, 4H), 1.05 (s, 9H).

MS obsd. (ESI⁺) [(M+H)⁺]: 556.3.

### Example 6

### N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide

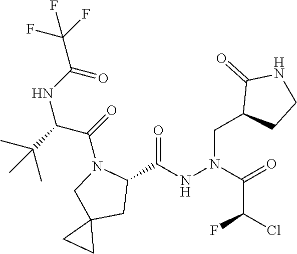 [SMILES: CC(C)(C)[C@H](NC(=O)C(F)(F)F)C(=O)N1CC2(CC2)C[C@H]1C(=O)NN(C[C@@H]1CCNC1=O)C(=O)[C@H](F)Cl]

The title compound was prepared in analogy to the procedure described for the preparation of Example 1, by using benzyl (6S)-5-[(2S)-2-amino-3,3-dimethyl-butanoyl]-5-azaspiro[2.4]heptane-6-carboxylate (Intermediate 5) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide (Example 6) as an off-white solid.

¹H NMR (400 MHz, CD₃OD) δ: ppm 7.25-6.78 (d, 1H), 4.68-4.60 (m, 1H), 4.48 (t, J=8.0 Hz, 1H), 3.98 (dd, J=8.4, 14.4 Hz, 1H), 3.87 (d, J=10 Hz, 1H), 3.66-3.56 (m, 2H), 3.41-3.34 (m, 2H), 2.69 (dt, J=4.0, 8.8 Hz, 1H), 2.34-2.16 (m, 2H), 2.07-1.96 (m, 2H), 1.08 (s, 9H), 0.81-0.60 (m, 4H)

MS obsd. (ESI⁺) [(M+H)⁺]: 556.3.

### Example 7

### N-[(1S)-1-[(2S,4R)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide

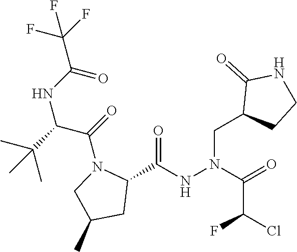 [SMILES: C[C@@H]1C[C@@H](C(=O)NN(C[C@@H]2CCNC2=O)C(=O)[C@H](F)Cl)N(C(=O)[C@@H](NC(=O)C(F)(F)F)C(C)(C)C)C1]

The title compound was prepared in analogy to the procedure described for the preparation of Example 1, by using benzyl (2S,4R)-1-[(2S)-2-amino-3,3-dimethyl-butanoyl]-4-methyl-pyrrolidine-2-carboxylate (Intermediate 6) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(2S,4R)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide (Example 7) as a white solid.

¹H NMR (400 MHz, CD₃OD) δ: ppm 6.98 (d, J=56.0 Hz, 1H), 4.85-4.86 (m, 1H), 4.47-4.37 (m, 1H), 4.06-3.94 (m, 2H), 3.67-3.56 (m, 1H), 3.48-3.34 (m, 3H), 2.78-2.68 (m, 1H), 2.66-2.54 (m, 1H), 2.35-2.23 (m, 1H), 2.23-2.14 (m, 1H), 2.11-1.86 (m, 2H), 1.12-1.04 (m, 12H).

MS obsd. (ESI⁺) [(M+H)⁺]: 544.2.

### Example 8

### N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide

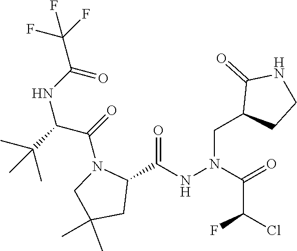 [SMILES: CC1(C)C[C@@H](C(=O)NN(C[C@@H]2CCNC2=O)C(=O)[C@H](F)Cl)N(C(=O)[C@@H](NC(=O)C(F)(F)F)C(C)(C)C)C1]

The title compound was prepared in analogy to the procedure described for the preparation of Example 1, by using benzyl (2S)-1-[(2S)-2-amino-3,3-dimethyl-butanoyl]-4,4-dimethyl-pyrrolidine-2-carboxylate (Intermediate 7) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide (Example 8) as a white solid.

¹H NMR (400 MHz, CD₃OD) δ: ppm 7.10 (d, J=50.4 Hz, 1H), 4.49-4.34 (m, 1H), 4.09-3.93 (m, 1H), 3.82-3.74 (m, 1H), 3.64-3.55 (m, 1H), 3.49-3.44 (m, 1H), 3.41-3.34 (m, 2H), 2.74-2.65 (m, 1H), 2.33-2.25 (m, 1H), 2.13-1.96 (m, 2H), 1.90-1.79 (m, 1H), 1.23 (s, 3H), 1.11 (s, 3H), 1.08 (s, 9H).

MS obsd. (ESI⁺) [(M+H)⁺]: 558.2.

### Example 9

### N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]-2,2-dimethyl-propyl]-2,2,2-trifluoro-acetamide

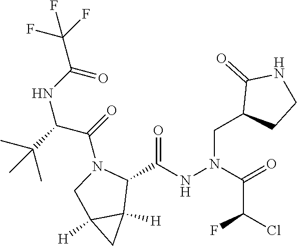 [SMILES: CC(C)(C)[C@H](NC(=O)C(F)(F)F)C(=O)N1C[C@@H]2C[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (1S,2S,5R)-3-[(2S)-2-amino-3,3-dimethyl-butanoyl]-3-azabicyclo[3.1.0]hexane-2-carboxylate (Intermediate 8) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]-2,2-dimethyl-propyl]-2,2,2-trifluoro-acetamide (Example 9) as a white solid.

¹H NMR (400 MHz, CDCl₃) δ: ppm 10.15 (s, 1H), 7.33 (s, 1H), 6.61-6.48 (m, 2H), 4.95 (s, 1H), 4.63 (d, J=9.6 Hz, 1H), 4.43-4.39 (m, 1H), 4.05-3.97 (m, 2H), 3.44-3.41 (m, 2H), 2.96-2.88 (m, 2H), 2.42-2.36 (m, 1H), 1.78-1.64 (m, 3H), 1.00 (s, 9H), 0.89 (q, J₁=7.6 Hz, J₂=13.6 Hz, 1H), 0.25 (d, J=4.4 Hz, 1H)

MS obsd. (ESI⁺) [(M+H)⁺]: 541.9.

### Example 10

### N-[(1S)-1-[(1R,2S,5S)-2-[[[(2R)-2-chloro-2-fluoro-acetyl]-(1H-pyrazol-3-ylmethyl)amino]carbamoyl]-6,6-dimethyl-3-azabicyclo[3.1.0]hexane-3-carbonyl]-2,2-dimethyl-propyl]-2,2,2-trifluoro-acetamide

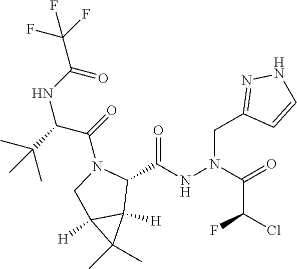 [SMILES: CC(C)(C)[C@H](NC(=O)C(F)(F)F)C(=O)N1C[C@H]2[C@@H]([C@H]1C(=O)NN(Cc1cc[nH]n1)C(=O)[C@H](F)Cl)C2(C)C]

The title compound was prepared in analogy to the procedure described for the preparation of Example 1, by using tert-butyl N-amino-N-(1H-pyrazol-3-ylmethyl)carbamate (Intermediate 11) instead of tert-butyl N-amino-N-[[(3S)-2-oxopyrrolidin-3-yl]methyl]carbamate (Intermediate 9 in step 3) to afford N-[(1S)-1-[(1R,2S,5S)-2-[[[(2R)-2-chloro-2-fluoro-acetyl]-(1H-pyrazol-3-ylmethyl)amino]carbamoyl]-6,6-dimethyl-3-azabicyclo[3.1.0]hexane-3-carbonyl]-2,2-dimethyl-propyl]-2,2,2-trifluoro-acetamide (Example 10) as a white solid.

¹H NMR (400 MHz, CD₃OD) δ: ppm 10.44-9.88 (m, 1H), 7.52-7.50 (m, 1H), 7.10-7.25 (m, 1H), 6.64-6.52 (m, 1H), 6.39 (s, 1H), 5.39-4.94 (m, 1H), 4.59 (d, J=9.6 Hz, 2H), 4.22-4.03 (m, 3H), 3.87 (d, J=10.0 Hz, 1H), 1.60-1.68 (m, 1H), 1.13-1.37 (m, 0.5H), 1.03 (br, s, 12H), 0.98 (br, s, 0.5H), 0.82 (s, 3H)

MS obsd. (ESI⁺) [(M+H)⁺]: 553.2.

### Example 11

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide

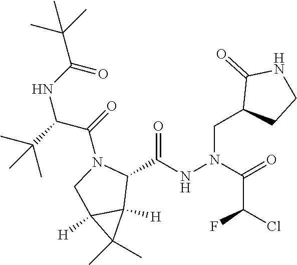 [SMILES: CC(C)(C)C(=O)N[C@H](C(=O)N1C[C@H]2[C@@H]([C@H]1C(=O)NN(C[C@@H]1CCNC1=O)C(=O)[C@H](F)Cl)C2(C)C)C(C)(C)C]

The title compound was prepared in analogy to the procedure described for the preparation of Example 1, by using 2,2-dimethylpropanoyl chloride instead of TFAA and DCM instead of THF to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide (Example 11) as an off-white solid.

¹H NMR (400 MHz, CD₃OD) δ: ppm 6.92-6.79 (m, 2H), 4.63 (d, J=9.4 Hz, 1H), 4.25 (s, 1H), 4.09-4.01 (m, 2H), 3.94 (br d, J=10.4 Hz, 1H), 3.66-3.57 (m, 1H), 3.43-3.35 (m, 2H), 2.86-2.64 (m, 1H), 2.37-2.22 (m, 1H), 2.11-1.98 (m, 1H), 1.73-1.64 (m, 2H), 1.21 (s, 9H), 1.12 (s, 3H), 1.03 (s, 9H), 0.96 (s, 3H)

MS obsd. (ESI⁺) [(M+Na)⁺]: 558.2.

### Example 12

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]cyclopropanecarboxamide

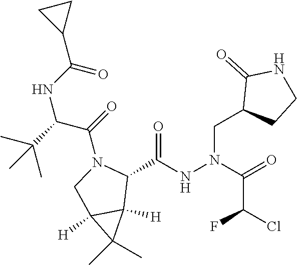 [SMILES: CC(C)(C)[C@H](NC(=O)C1CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 1, by using cyclopropanecarbonyl chloride instead of TFAA to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]cyclopropanecarboxamide (Example 12) as a white solid.

¹H NMR (400 MHz, CDCl₃) δ: ppm 10.13 (d, J=3.2 Hz, 1H), 6.86-6.65 (m, 1H), 6.64-6.47 (m, 1H), 6.21-6.15 (m, 1H), 4.67 (d, J=10.0 Hz, 1H), 4.56-4.22 (m, 2H), 4.06-3.98 (m, 1H), 3.98-3.91 (m, 1H), 3.41 (dd, J=5.2, 8.8 Hz, 2H), 3.19-2.64 (m, 2H), 2.47-2.34 (m, 1H), 1.81 (d, J=6.0 Hz, 1H), 1.56 (m, 4H), 1.34 (d, J=6.4 Hz, 1H), 1.05 (s, 3H), 1.02-0.93 (m, 9H), 0.89 (s, 3H), 0.80-0.68 (m, 2H)

MS obsd. (ESI⁺) [(M+H)⁺]: 542.3.

### Example 13

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide

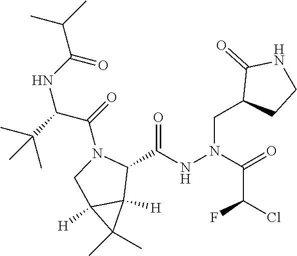 [SMILES: CC(C)C(=O)N[C@H](C(=O)N1C[C@H]2[C@@H]([C@H]1C(=O)NN(C[C@@H]1CCNC1=O)C(=O)[C@H](F)Cl)C2(C)C)C(C)(C)C]

The title compound was prepared in analogy to the procedure described for the preparation of Example 1, by using 2-methylpropanoyl chloride instead of TFAA to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide (Example 13) as a white solid.

¹H NMR (400 MHz, CD₃OD) δ: ppm 6.92 (d, J=50.8 Hz, 1H), 4.54 (s, 1H), 4.24 (s, 1H), 4.09-3.94 (m, 3H), 3.63-3.57 (m, 1H), 3.43-3.35 (m, 2H), 2.80-2.69 (m, 1H), 2.66-2.54 (m, 1H), 2.37-2.25 (m, 1H), 2.11-1.97 (m, 1H), 1.74-1.63 (m, 2H), 1.16-1.11 (m, 6H), 1.09-1.03 (m, 12H), 0.99 (s, 3H).

MS obsd. (ESI⁺) [(M+H)⁺]: 544.4.

### Example 14

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide

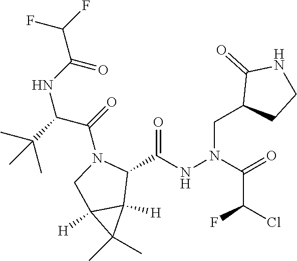 [SMILES: CC(C)(C)[C@H](NC(=O)C(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 1, by using (2,2-difluoroacetyl) 2,2-difluoroacetate instead of TFAA to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide (Example 14) as an off-white solid.

¹H NMR (400 MHz, CD₃OD) δ: ppm 7.00-6.79 (m, 1H), 6.30-5.97 (m, 1H), 4.53 (s, 1H), 4.26 (s, 1H), 4.10-4.00 (m, 2H), 3.93 (d, J=10.4 Hz, 1H), 3.59 (br dd, J=4.4, 14.4 Hz, 1H), 3.42-3.34 (m, 2H), 2.75 (dq, J=4.4, 8.8 Hz, 1H), 2.37-2.25 (m, 1H), 2.09-1.98 (m, 1H), 1.75-1.63 (m, 2H), 1.12 (s, 3H), 1.08 (s, 9H), 1.01 (s, 3H).

MS obsd. (ESI⁺) [(M+H)⁺]: 552.3.

### Example 15

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide

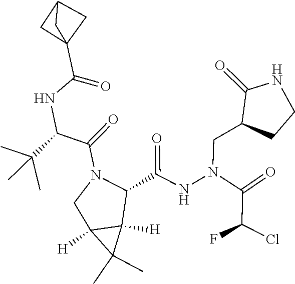 [SMILES: CC(C)(C)[C@H](NC(=O)C12CC(C1)C2)C(=O)N1C[C@H]2[C@@H]([C@H]1C(=O)NN(C[C@@H]1CCNC1=O)C(=O)[C@H](F)Cl)C2(C)C]

### Step 1: Preparation of benzyl (1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carboxylate

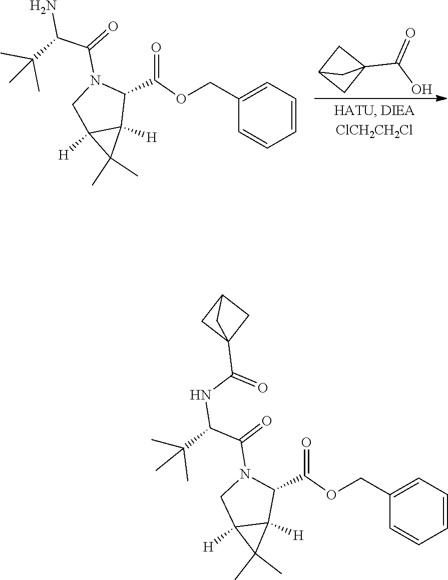

To a solution of bicyclo[1.1.1]pentane-1-carboxylic acid (75 mg, 0.67 mmol) in 1,2-dichloroethane (10 mL) was added N,N-diisopropylethylamine (0.33 mL, 1.9 mmol), 0-(7-azabenzotriazol-1-yl)-N,N,N′,N′-tetramethyluronium hexafluorophosphate (290 mg, 0.76 mmol) and benzyl (1R,2S,5S)-3-[(2S)-2-amino-3,3-dimethyl-butanoyl]-6,6-dimethyl-3-azabicyclo[3.1.0]hexane-2-carboxylate (229 mg, 0.638 mmol, Intermediate 1). The reaction mixture was stirred at 25° C. for 1 h. The mixture was concentrated and the residue was purified by silica gel column eluted with PE/EtOAc=20/1 to 10/1 to afford benzyl (1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carboxylate (230 mg) as colorless oil.

MS obsd. (ESI⁺) [(M+H)⁺]: 453.3.

### Step 2: Preparation of (1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carboxylic acid

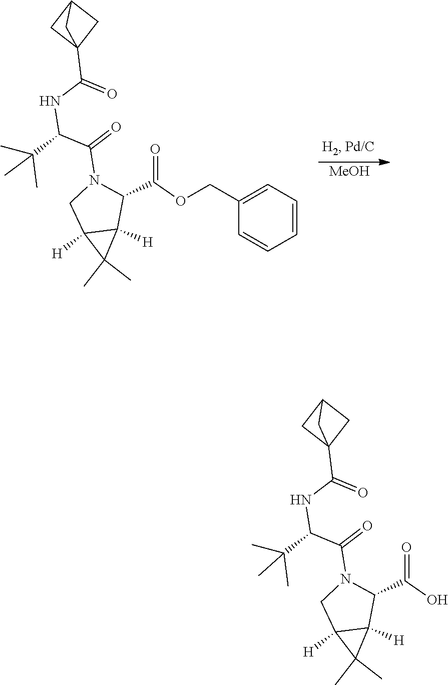 [SMILES: "MeOH" ; CC(C)(C)[C@H](NC(=O)C12CC(C1)C2)C(=O)N1C[C@H]2[C@@H]([C@H]1C(=O)OCc1ccccc1)C2(C)C ; CC(C)(C)[C@H](NC(=O)C12CC(C1)C2)C(=O)N1C[C@H]2[C@@H]([C@H]1C(=O)O)C2(C)C]

To a solution of benzyl (1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carboxylate (230 mg, 0.51 mmol) in methanol (10 mL) was added Pd/C (100 mg, 10% purity). The reaction mixture was degassed under vacuum and purged H₂ for 3 times. The resulting mixture was stirred at 25° C. for 1 h under H₂ balloon. The suspension was filtered and the filtrate was concentrated in vacuum to afford (1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carboxylic acid (170 mg) as colorless oil.

MS obsd. (ESI⁺) [(M+H)⁺]: 363.2.

### Step 3: Preparation of tert-butyl N-[[(1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate

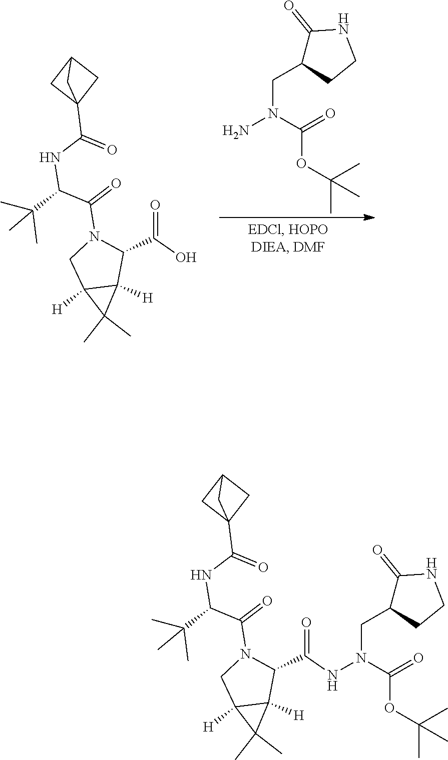 [SMILES: CC(C)(C)[C@H](NC(=O)C12CC(C1)C2)C(=O)N1C[C@H]2[C@@H]([C@H]1C(=O)O)C2(C)C ; CC(C)(C)OC(=O)N(N)C[C@@H]1CCNC1=O ; CC(C)(C)OC(=O)N(C[C@@H]1CCNC1=O)NC(=O)[C@@H]1[C@@H]2[C@H](CN1C(=O)[C@@H](NC(=O)C13CC(C1)C3)C(C)(C)C)C2(C)C]

To a solution of (1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carboxylic acid (170 mg, 0.470 mmol) in DMF (2 mL) was added EDCI (108 mg, 0.56 mmol), HOPO (62 mg, 0.56 mmol), DIPEA (0.41 mL, 2.35 mmol) and tert-butyl N-amino-N-[[(3S)-2-oxopyrrolidin-3-yl]methyl]carbamate (118 mg, 0.520 mmol, Intermediate 9). The reaction mixture was stirred at 25° C. for 12 h. The mixture was diluted with EtOAc (30 mL), washed with water (20 mL) and brine (20 mL×3). The combined organic phase was dried over Na₂SO₄, filtered and concentrated. The residue was purified by reverse phase (neutral condition) eluted with ACN in H₂O=0˜70% to afford tert-butyl N-[[(1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (210 mg) as a white solid.

MS obsd. (ESI⁺) [(M+H)⁺]: 574.3.

### Step 4: Preparation of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]bicyclo[1.1.1]pentane-1-carboxamide

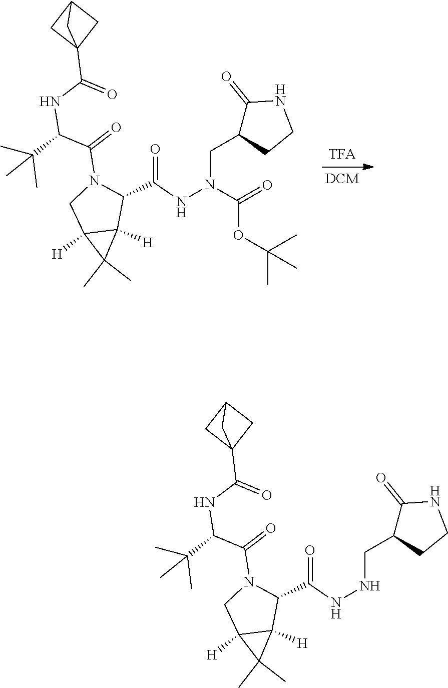

To a solution of tert-butyl N-[[(1R,2S,5S)-3-[(2S)-2-(bicyclo[1.1.1]pentane-1-carbonylamino)-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (210 mg, 0.37 mmol) in DCM (2.8 mL) was added TFA (2.82 mL). The reaction mixture was stirred at 25° C. for 1 h. The residue was purified by reverse phase ACN in H₂O (0.1% HCl)=0˜70% to afford N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]bicyclo[1.1.1]pentane-1-carboxamide (60 mg) as a yellow solid.

MS obsd. (ESI⁺) [(M+H)⁺]: 474.4.

### Step 5: Preparation of N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide (Example 15)

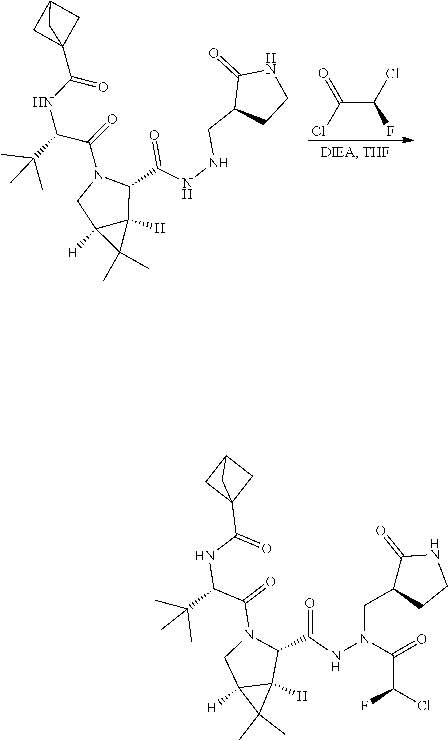

To a solution of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]bicyclo[1.1.1]pentane-1-carboxamide (60 mg, 0.120 mmol) in THF (10 mL) was added DIPEA (0.25 mL, 1.41 mmol) and (2R)-2-chloro-2-fluoro-acetyl chloride (77 mg, 0.590 mmol, Intermediate 10). The reaction mixture was stirred at 0° C. for 1 h. The mixture was concentrated and the residue was purified by prep-HPLC (0.10% TFA, 3_Phenomenex Luna C18 75*30 mm*3 um; mobile phase: [water (0.1% TFA)-ACN]; B %: 45%-65%, 9 min) to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide (31 mg, Example 15) as a white solid.

¹H NMR (400 MHz, CD₃OD) δ: ppm 6.97-6.74 (d, J=50.8 Hz, 1H), 4.60-4.51 (m, 1H), 4.24 (s, 1H), 4.10-3.98 (m, 2H), 3.94-3.85 (m, 1H), 3.67-3.58 (m, 1H), 3.46-3.36 (m, 2H), 2.81-2.67 (m, 1H), 2.50-2.40 (m, 1H), 2.36-2.26 (m, 1H), 2.09 (s, 6H), 2.04-1.96 (m, 1H), 1.72-1.64 (m, 2H), 1.12 (s, 3H), 1.03 (s, 9H), 0.97 (s, 3H).

MS obsd. (ESI⁺) [(M+H)⁺]: 568.4.

### Example 16

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-5-(trifluoromethyl)isoxazole-3-carboxamide

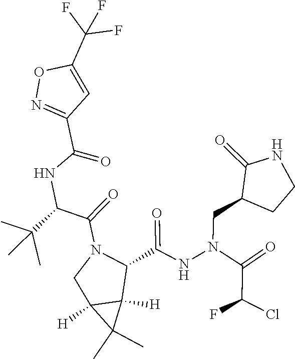 [SMILES: CC(C)(C)[C@H](NC(=O)c1cc(C(F)(F)F)on1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 1, by using 5-(trifluoromethyl)isoxazole-3-carboxylic acid (Intermediate 12) instead of bicyclo[1.1.1]pentane-1-carboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-5-(trifluoromethyl)isoxazole-3-carboxamide (Example 16) as a yellow solid.

¹H NMR (400 MHz, CDCl₃) δ: ppm 10.03-9.78 (m, 1H), 7.69-7.46 (m, 1H), 7.11 (s, 1H), 6.78-6.56 (m, 1H), 6.16-5.90 (m, 1H), 4.72 (d, J=9.6 Hz, 1H), 4.54-4.28 (m, 2H), 4.07 (dd, J=5.2, 10.0 Hz, 1H), 4.00-3.90 (m, 1H), 3.42 (s, 2H), 3.09-2.65 (m, 2H), 2.50-2.29 (m, 1H), 1.90-1.79 (m, 1H), 1.63 (s, 1H), 1.43-1.33 (m, 1H), 1.08-1.03 (m, 12H), 0.93 (s, 3H).

MS obsd. (ESI⁺) [(M+H)⁺]: 637.2.

### Example 17

### (1R,2S,5S)—N′-[(2R)-2-chloro-2-fluoro-acetyl]-3-[2-(3,5-difluorophenoxy)acetyl]-6,6-dimethyl-N′—[[(3S)-2-oxopyrrolidin-3-yl]methyl]-3-azabicyclo[3.1.0]hexane-2-carbohydrazide

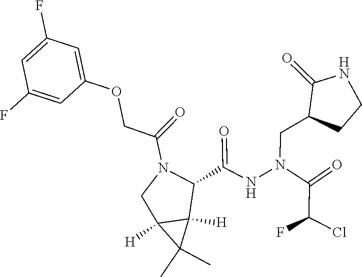 [SMILES: CC1(C)[C@@H]2[C@@H](C(=O)NN(C[C@@H]3CCNC3=O)C(=O)[C@H](F)Cl)N(C(=O)COc3cc(F)cc(F)c3)C[C@@H]21]

### Step 1: Preparation of (1R,2S,5S)-3-[2-(3,5-difluorophenoxy)acetyl]-6,6-dimethyl-3-azabicyclo[3.1.0]hexane-2-carboxylic acid

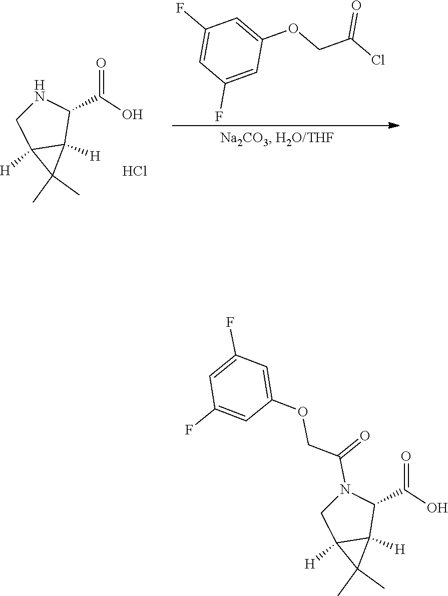 [SMILES: CC1(C)[C@@H]2[C@@H](C(=O)O)NC[C@@H]21 ; Cl ; O=C(Cl)COc1cc(F)cc(F)c1 ; CC1(C)[C@@H]2[C@@H](C(=O)O)N(C(=O)COc3cc(F)cc(F)c3)C[C@@H]21]

To a solution of (1R,2S,5S)-6,6-dimethyl-3-azabicyclo[3.1.0]hexane-2-carboxylic acid; hydrochloride (102 mg, 0.530 mmol) in water (2 mL) was added Na₂CO₃ (226 mg, 2.13 mmol) and the solution of 2-(3,5-difluorophenoxy)acetyl chloride (110 mg, 0.530 mmol, Intermediate 13) in THF (5 mL). The mixture was stirred at 25° C. for 5 h. The mixture was diluted with H₂O (30 mL) and washed with EtOAc (20 mL). The aqueous phase was acidified with concentrated HCl to pH=5. The suspension was extracted with EtOAc (30 mL×3). The combined organic phase was dried over Na₂SO₄, filtrated and the filtrate was concentrated to afford (1R,2S,5S)-3-[2-(3,5-difluorophenoxy)acetyl]-6,6-dimethyl-3-azabicyclo[3.1.0]hexane-2-carboxylic acid (150 mg) as a white solid.

MS obsd. (ESI⁺) [(M+H)⁺]: 326.1.

### Step 2: Preparation of tert-butyl N-[[(1R,2S,5S)-3-[2-(3,5-difluorophenoxy)acetyl]-6,6-dimethyl-3-azabicyclo[3.1.0]hexane-2-carbonyl]amino]-N-[[(3S)-2-oxopyrrolidin-3-yl]methyl]carbamate

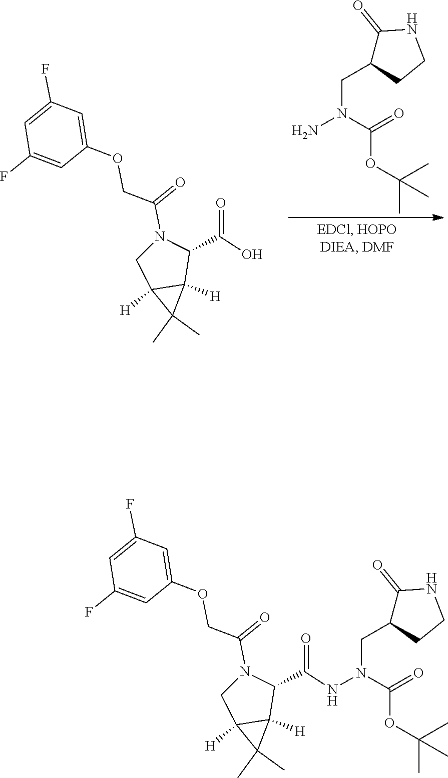 [SMILES: CC1(C)[C@@H]2[C@@H](C(=O)O)N(C(=O)COc3cc(F)cc(F)c3)C[C@@H]21 ; CC(C)(C)OC(=O)N(N)C[C@@H]1CCNC1=O ; CC(C)(C)OC(=O)N(C[C@@H]1CCNC1=O)NC(=O)[C@@H]1[C@@H]2[C@H](CN1C(=O)COc1cc(F)cc(F)c1)C2(C)C]

To a solution of (1R,2S,5S)-3-[2-(3,5-difluorophenoxy)acetyl]-6,6-dimethyl-3-azabicyclo[3.1.0]hexane-2-carboxylic acid (110 mg, 0.340 mmol) in DMF (10 mL) was added DIPEA (131 mg, 1.01 mmol), tert-butyl N-amino-N-[[(3S)-2-oxopyrrolidin-3-yl]methyl]carbamate (78 mg, 0.340 mmol, Intermediate 9), EDCI (155 mg, 0.410 mmol) and HOPO (45 mg, 0.410 mmol). The mixture was stirred at 25° C. for 2 h. The reaction mixture was diluted with EtOAc (40 ml) and poured into water (50 mL). After separation, the aqueous phase was extracted with EtOAc (30 mL×3). The combined organic layers were dried over Na₂SO₄. After filtration and concentration, the residue was purified by silica gel column eluted with PE/EtOAc=10/1 to 4/1 to afford tert-butyl N-[[(1R,2S,5S)-3-[2-(3,5-difluorophenoxy)acetyl]-6,6-dimethyl-3-azabicyclo[3.1.0]hexane-2-carbonyl]amino]-N-[[(3S)-2-oxopyrrolidin-3-yl]methyl]carbamate (150 mg) as colorless oil.

MS obsd. (ESI⁺) [(M+H)⁺]: 537.4.

### Step 3: Preparation of (1R,2S,5S)-3-[2-(3,5-difluorophenoxy)acetyl]-6,6-dimethyl-N′—[[(3S)-2-oxopyrrolidin-3-yl]methyl]-3-azabicyclo[3.1.0]hexane-2-carbohydrazide

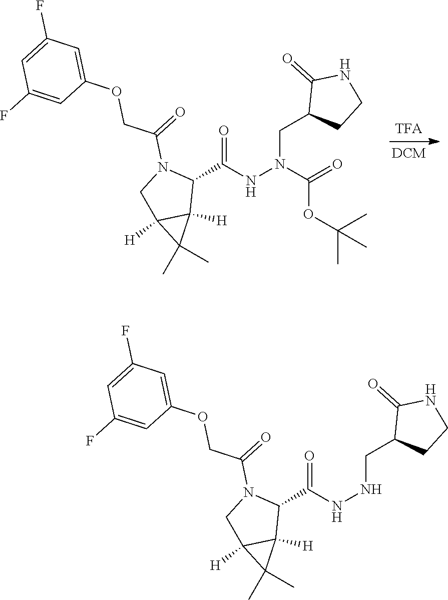 [SMILES: CC(C)(C)OC(=O)N(C[C@@H]1CCNC1=O)NC(=O)[C@@H]1[C@@H]2[C@H](CN1C(=O)COc1cc(F)cc(F)c1)C2(C)C ; CC1(C)[C@@H]2[C@@H](C(=O)NNC[C@@H]3CCNC3=O)N(C(=O)COc3cc(F)cc(F)c3)C[C@@H]21]

To a solution of tert-butyl N-[[(1R,2S,5S)-3-[2-(3,5-difluorophenoxy)acetyl]-6,6-dimethyl-3-azabicyclo[3.1.0]hexane-2-carbonyl]amino]-N-[[(3S)-2-oxopyrrolidin-3-yl]methyl]carbamate (120 mg, 0.220 mmol) in DCM (3 mL) was added TFA (1.0 mL). The mixture was stirred at 25° C. for 1 h. The reaction mixture was concentrated and the residue was purified with reverse flash eluted with ACN in H₂O (0.01% HCl)=0˜60% to afford (1R,2S,5S)-3-[2-(3,5-difluorophenoxy)acetyl]-6,6-dimethyl-N-[[(3S)-2-oxopyrrolidin-3-yl]methyl]-3-azabicyclo[3.1.0]hexane-2-carbohydrazide (70 mg) as a white solid.

### Step 4: Preparation of (1R,2S,5S)—N-[(2R)-2-chloro-2-fluoro-acetyl]-3-[2-(3,5-difluorophenoxy)acetyl]-6,6-dimethyl-N-[[(3S)-2-oxopyrrolidin-3-yl]methyl]-3-azabicyclo[3.1.0]hexane-2-carbohydrazide (Example 17)

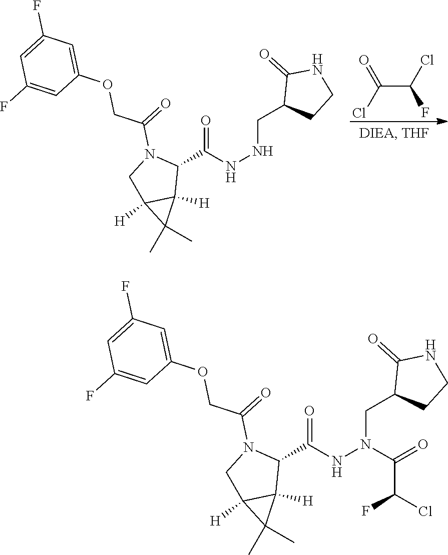

To a solution of (1R,2S,5S)-3-[2-(3,5-difluorophenoxy)acetyl]-6,6-dimethyl-N-[[(3S)-2-oxopyrrolidin-3-yl]methyl]-3-azabicyclo[3.1.0]hexane-2-carbohydrazide (60 mg, 0.140 mmol) in THF (14 mL) was added DIPEA (179 mg, 1.39 mmol) and a solution of (2R)-2-chloro-2-fluoro-acetyl chloride (90 mg, 0.320 mmol, Intermediate 10) in THF (4 mL) successively at −20° C. The mixture was stirred at 0° C. for 30 min. The reaction mixture was quenched with MeOH (2 mL), adjusted with a solution of 4M HCl in dioxane to pH=5 at 0° C. and concentrated in vacuum. The residue was purified by prep-HPLC (Unisil 3-100 C18 Ultra 150*50 mm*3 um; ACN in water (0.1% FA); 30%-60%; Flow Rate (mL/min):25) to afford (1R,2S,5S)—N-[(2R)-2-chloro-2-fluoro-acetyl]-3-[2-(3,5-difluorophenoxy)acetyl]-6,6-dimethyl-N-[[(3S)-2-oxopyrrolidin-3-yl]methyl]-3-azabicyclo[3.1.0]hexane-2-carbohydrazide (21 mg, Example 17) as a white solid.

¹H NMR (400 MHz, CD₃OD) δ: ppm 10.02 (s, 1H), 6.49-6.45 (m, 3H), 6.42-6.29 (m, 1H), 5.67 (s, 1H), 4.75-4.72 (m, 1H), 4.59-4.62 (m, 1H), 4.42-4.35 (m, 2H), 3.97-3.93 (m, 1H), 3.65-3.63 (m, 1H), 3.41-3.38 (m, 2H), 2.92-2.79 (m, 2H), 2.37-2.30 (m, 1H), 1.85-1.75 (m, 2H), 1.51-1.49 (m, 1H), 1.11 (s, 3H), 0.94 (s, 3H).

MS obsd. (ESI⁺) [(M+H)⁺]: 530.9.

### Example 18

### N-[(1S)-1-[(1R,2S,5S)-2-[[(2-chloroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide

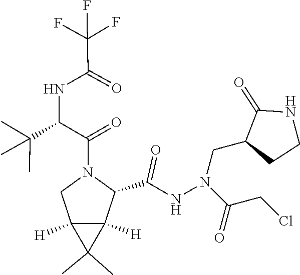 [SMILES: CC(C)(C)[C@H](NC(=O)C(F)(F)F)C(=O)N1C[C@H]2[C@@H]([C@H]1C(=O)NN(C[C@@H]1CCNC1=O)C(=O)CCl)C2(C)C]

The title compound was prepared in analogy to the procedure described for the preparation of Example 1, by using chloroacetyl chloride instead of (2R)-2-chloro-2-fluoro-acetyl chloride (Intermediate 10) to afford N-[(1S)-1-[(1R,2S,5S)-2-[[(2-chloroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide (Example 18) as a white solid.

¹H NMR (400 MHz, DMSO-d₆) δ: ppm 10.98-10.62 (m, 1H), 9.56-9.37 (m, 1H), 7.82-7.61 (m, 1H), 4.51-4.37 (m, 2H), 4.28-4.03 (m, 2H), 3.92 (dd, J=5.6, 10.4 Hz, 1H), 3.83-3.66 (m, 2H), 3.22-3.10 (m, 2H), 2.69-2.65 (m, 1H), 2.37-2.30 (m, 1H), 2.24-2.06 (m, 1H), 1.84-1.68 (m, 1H), 1.62 (dd, J=5.6, 7.4 Hz, 1H), 1.56-1.43 (m, 1H), 1.04 (s, 3H), 0.99 (s, 9H), 0.89 (s, 3H).

MS obsd. (ESI⁺) [(M+H)⁺]: 552.0.

### Example 19

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide

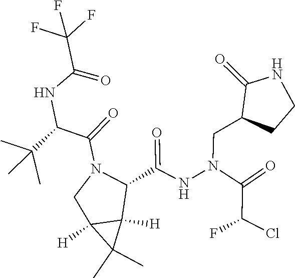 [SMILES: CC(C)(C)[C@H](NC(=O)C(F)(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 1, by using 2-chloro-2-fluoro-acetyl chloride (Intermediate 10-rac) instead of (2R)-2-chloro-2-fluoro-acetyl chloride (Intermediate 10) and purified by SFC separation (Column: REGIS(S,S)WHELK-O1(250 mm*25 mm, 10 um); Condition Neu-IPA Begin B 25% End B 25%; Gradient Time(min) 2.8; 100% B Hold Time(min) 5; Rate(ml/min) 50; Injections 135) to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide (Example 19) as a white solid.

¹H NMR (400 MHz, DMSO-d₆) δ: ppm 10.98 (s, 1H), 9.46 (d, J=7.2 Hz, 1H), 7.84-7.53 (m, 1H) 6.83 (d, J=48.8 Hz, 1H), 4.41 (d, J=6.0 Hz, 1H), 4.26-4.13 (m, 1H), 3.93 (dd, J=10.4, 5.2 Hz, 1H), 3.84-3.63 (m, 2H), 3.52 (d, J=13.20 Hz, 1H), 3.20-3.11 (in, 1H), 3.05-2.95 (m, 1H), 2.64-2.55 (m, 2H), 2.23-2.10 (m, 1H), 1.85-1.71 (m, 1H), 1.68-1.44 (m, 2H), 1.07-1.03 (m, 3H) 1.01-0.96 (m, 8H), 0.89 (s, 3H).

MS obsd. (ESI⁺) [(M+H)⁺]: 570.3.

### Example 20

### N-[(1S)-1-benzyl-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide

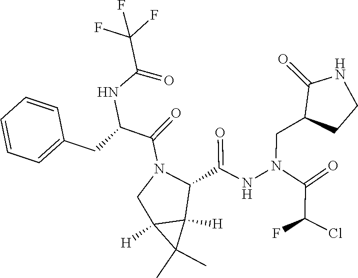 [SMILES: CC1(C)[C@@H]2[C@@H](C(=O)NN(C[C@@H]3CCNC3=O)C(=O)[C@H](F)Cl)N(C(=O)[C@H](Cc3ccccc3)NC(=O)C(F)(F)F)C[C@@H]21]

### Step 1: Preparation of tert-butyl N-[[(1R,2S,5S)-3-[(2S)-2-(benzyloxycarbonylamino)-3-phenyl-propanoyl]-6,6-dimethyl-3-azabicyclo[3.1.0]hexane-2-carbonyl]amino]-N-[[(3S)-2-oxopyrrolidin-3-yl]methyl]carbamate

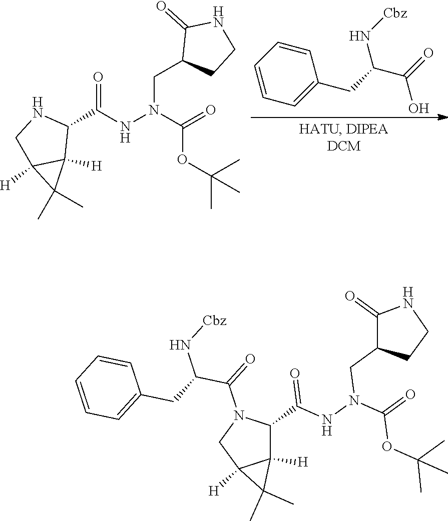

To a solution of Cbz-Phe-OH (245 mg, 0.820 mmol) (GL Biochem, CAS number: 1161-13-3) in DCM (6 mL) was added DIPEA (0.43 mL, 2.46 mmol), HATU (231 mg, 0.98 mmol) and tert-butyl N-[[(1R,2S,5S)-6,6-dimethyl-3-azabicyclo[3.1.0]hexane-2-carbonyl]amino]-N-[[(3S)-2-oxopyrrolidin-3-yl]methyl]carbamate (300 mg, 0.820 mmol, Intermediate 14). The mixture was stirred at 25° C. for 2 h. The mixture was diluted with water (40 mL) and extracted with ethyl acetate (50 mL×2). The combined organic phase was washed with brine (50 mL×2), dried over anhydrous Na₂SO₄, filtered and concentrated in vacuum. The residue was purified by silica gel column eluted with PE/EtOAc=10/1 to 2/1 to afford tert-butyl N-[[(1R,2S,5S)-3-[(2S)-2-(benzyloxycarbonylamino)-3-phenyl-propanoyl]-6,6-dimethyl-3-azabicyclo[3.1.0]hexane-2-carbonyl]amino]-N-[[(3S)-2-oxopyrrolidin-3-yl]methyl]carbamate (350 mg) as yellow oil.

MS obsd. (ESI⁺) [(M+H)⁺]: 648.4.

### Step 2: Preparation of tert-butyl N-[[(1R,2S,5S)-3-[(2S)-2-amino-3-phenyl-propanoyl]-6,6-dimethyl-3-azabicyclo[3.1.0]hexane-2-carbonyl]amino]-N-[[(3S)-2-oxopyrrolidin-3-yl]methyl]carbamate

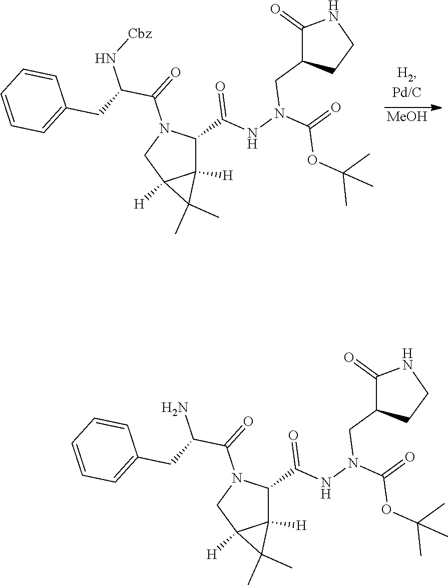 [SMILES: "MeOH" ; CC(C)(C)OC(=O)N(C[C@@H]1CCNC1=O)NC(=O)[C@@H]1[C@@H]2[C@H](CN1C(=O)[C@H](Cc1ccccc1)NC(=O)OCc1ccccc1)C2(C)C ; CC(C)(C)OC(=O)N(C[C@@H]1CCNC1=O)NC(=O)[C@@H]1[C@@H]2[C@H](CN1C(=O)[C@@H](N)Cc1ccccc1)C2(C)C]

To a solution of tert-butyl N-[[(1R,2S,5S)-3-[(2S)-2-(benzyloxycarbonylamino)-3-phenyl-propanoyl]-6,6-dimethyl-3-azabicyclo[3.1.0]hexane-2-carbonyl]amino]-N-[[(3S)-2-oxopyrrolidin-3-yl]methyl]carbamate (350 mg, 0.540 mmol) in methanol (8 mL) was added Pd/C (150 mg, 10% purity) under N₂ atmosphere. The suspension was degassed and purged with H₂ for 3 times. The mixture was stirred under a H₂ balloon at 25° C. for 2 h. The suspension was filtered and the filtrate was concentrated in vacuum to afford tert-butyl N-[[(1R,2S,5S)-3-[(2S)-2-amino-3-phenyl-propanoyl]-6,6-dimethyl-3-azabicyclo[3.1.0]hexane-2-carbonyl]amino]-N-[[(3S)-2-oxopyrrolidin-3-yl]methyl]carbamate (270 mg) as yellow oil.

MS obsd. (ESI⁺) [(M+H)⁺]: 514.3.

### Step 3: Preparation of tert-butyl N-[[(1R,2S,5S)-6,6-dimethyl-3-[(2S)-3-phenyl-2-[(2,2,2-trifluoroacetyl)amino]propanoyl]-3-azabicyclo[3.1.0]hexane-2-carbonyl]amino]-N-[[(3S)-2-oxopyrrolidin-3-yl]methyl]carbamate

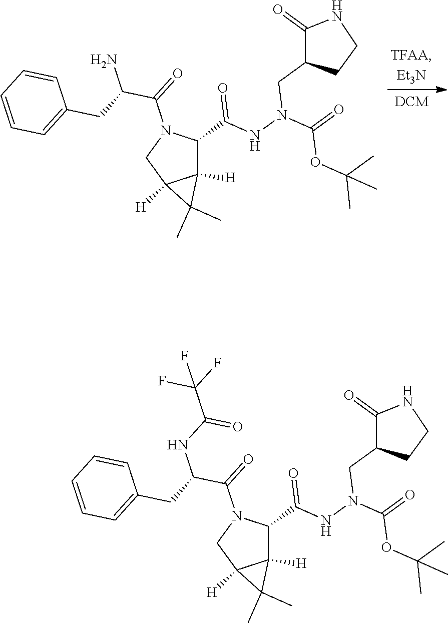 [SMILES: CC(C)(C)OC(=O)N(C[C@@H]1CCNC1=O)NC(=O)[C@@H]1[C@@H]2[C@H](CN1C(=O)[C@@H](N)Cc1ccccc1)C2(C)C ; CC(C)(C)OC(=O)N(C[C@@H]1CCNC1=O)NC(=O)[C@@H]1[C@@H]2[C@H](CN1C(=O)[C@H](Cc1ccccc1)NC(=O)C(F)(F)F)C2(C)C]

To a solution of tert-butyl N-[[(1R,2S,5S)-3-[(2S)-2-amino-3-phenyl-propanoyl]-6,6-dimethyl-3-azabicyclo[3.1.0]hexane-2-carbonyl]amino]-N-[[(3S)-2-oxopyrrolidin-3-yl]methyl]carbamate (270 mg, 0.530 mmol) in THF (8 mL) was added TEA (133 mg, 1.31 mmol) and TFAA (0.11 mL, 0.790 mmol) at 0° C. The reaction mixture was stirred at 25° C. for 1 h. The mixture was diluted with water (80 mL) and extracted with EtOAc (60 mL×3). The organic layers were washed with brine (80 mL) and dried over anhydrous sodium sulfate. After filtration and concentration, the residue was purified by silica gel column eluted with ethyl PE/EtOAc=20/1 to 9/1 to afford tert-butyl N-[[(1R,2S,5S)-6,6-dimethyl-3-[(2S)-3-phenyl-2-[(2,2,2-trifluoroacetyl)amino]propanoyl]-3-azabicyclo[3.1.0]hexane-2-carbonyl]amino]-N-[[(3S)-2-oxopyrrolidin-3-yl]methyl]carbamate (138 mg) as a white solid.

MS obsd. (ESI⁺) [(M+H)⁺]: 610.2.

### Step 4: Preparation of N-[(1S)-1-benzyl-2-[(1R,2S,5S)-6,6-dimethyl-2-[[[(3S)-2-oxopyrrolidin-3-yl]methylamino]carbamoyl]-3-azabicyclo[3.1.0]hexan-3-yl]-2-oxo-ethyl]-2,2,2-trifluoro-acetamide

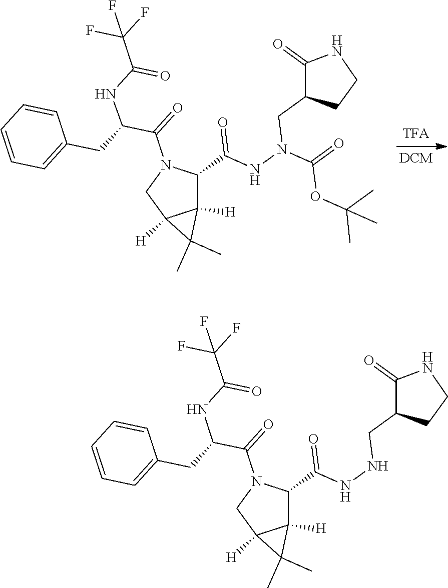 [SMILES: CC(C)(C)OC(=O)N(C[C@@H]1CCNC1=O)NC(=O)[C@@H]1[C@@H]2[C@H](CN1C(=O)[C@H](Cc1ccccc1)NC(=O)C(F)(F)F)C2(C)C ; CC1(C)[C@@H]2[C@@H](C(=O)NNC[C@@H]3CCNC3=O)N(C(=O)[C@H](Cc3ccccc3)NC(=O)C(F)(F)F)C[C@@H]21]

To a solution of tert-butyl N-[[(1R,2S,5S)-6,6-dimethyl-3-[(2S)-3-phenyl-2-[(2,2,2-trifluoroacetyl)amino]propanoyl]-3-azabicyclo[3.1.0]hexane-2-carbonyl]amino]-N-[[(3S)-2-oxopyrrolidin-3-yl]methyl]carbamate (138 mg, 0.230 mmol) in DCM (10 mL) was added TFA (5.0 mL). The mixture was stirred at 25° C. for 1 h. The mixture was concentrated in vacuum. The residue was purified with reverse flash eluted with ACN in H₂O (0.01% HCl)=0˜60% to afford N-[(1S)-1-benzyl-2-[(1R,2S,5S)-6,6-dimethyl-2-[[[(3S)-2-oxopyrrolidin-3-yl]methylamino]carbamoyl]-3-azabicyclo[3.1.0]hexan-3-yl]-2-oxo-ethyl]-2,2,2-trifluoro-acetamide (42 mg) as a white solid.

MS obsd. (ESI⁺) [(M+H)⁺]: 510.2.

### Step 5: Preparation of N-[(1S)-1-benzyl-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide (Example 21)

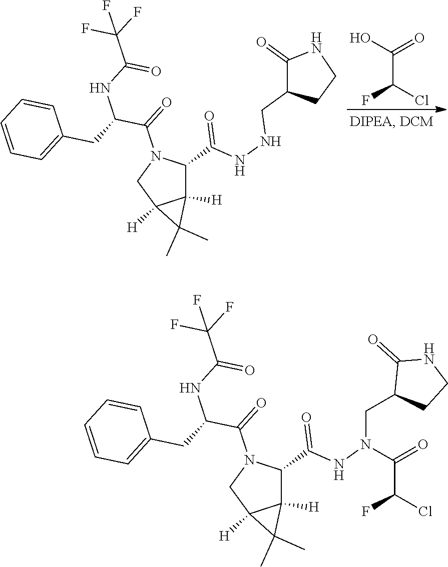

To a solution of N-[(1S)-1-benzyl-2-[(1R,2S,5S)-6,6-dimethyl-2-[[[(3S)-2-oxopyrrolidin-3-yl]methylamino]carbamoyl]-3-azabicyclo[3.1.0]hexan-3-yl]-2-oxo-ethyl]-2,2,2-trifluoro-acetamide (42 mg, 0.080 mmol) in THF (20 mL) was added DIPEA (127 mg, 0.990 mmol) and (2R)-2-chloro-2-fluoro-acetyl chloride (53.96 mg, 0.410 mmol, Intermediate 10) at 0° C. The reaction mixture was stirred at 0° C. for 1 h. The mixture was quenched with 4 M HCl/dioxane until pH=5˜6 and then concentrated in vacuum. The residue was purified by prep-HPLC (0.1% TFA, 3_Phenomenex Synergi Polar-RP 100*25 mm*4 um: [water (0.1% TFA)−ACN]; B %: 48%-68%, 7 min) to afford N-[(1S)-1-benzyl-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide (19 mg, Example 20) as an off-white solid.

¹H NMR (400 MHz, CD₃OD) δ: ppm 7.36-7.21 (m, 5H), 6.97-6.78 (m, 1H), 4.79 (dd, J=5.6, 8.8 Hz, 1H), 4.26 (s, 1H), 4.08 (dd, J=8.8, 14.0 Hz, 1H), 3.85-3.80 (m, 1H), 3.79-3.72 (m, 1H), 3.59 (dd, J=4.4, 14.0 Hz, 1H), 3.44-3.34 (m, 2H), 3.18 (dd, J=5.6, 14.0 Hz, 1H), 2.97 (dd, J=8.8, 14.0 Hz, 1H), 2.75 (dd, J=4.8, 8.4 Hz, 1H), 2.38-2.24 (m, 1H), 2.12-1.97 (m, 1H), 1.74-1.59 (m, 2H), 1.12 (s, 3H), 1.02 (s, 3H).

MS obsd. (ESI⁺) [(M+H)⁺]: 604.3.

### Example 21

### N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-phenyl-ethyl]-2,2,2-trifluoro-acetamide

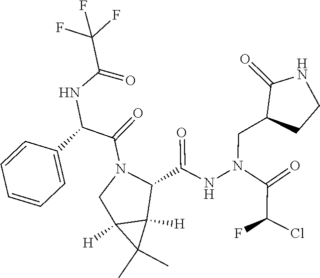 [SMILES: CC1(C)[C@@H]2[C@@H](C(=O)NN(C[C@@H]3CCNC3=O)C(=O)[C@H](F)Cl)N(C(=O)[C@@H](NC(=O)C(F)(F)F)c3ccccc3)C[C@@H]21]

The title compound was prepared in analogy to the procedure described for the preparation of Example 20, by using (2S)-2-(benzyloxycarbonylamino)-2-phenyl-acetic acid (GL Biochem, CAS number: 53990-33-3) instead of Cbz-Phe-OH to afford N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-phenyl-ethyl]-2,2,2-trifluoro-acetamide (Example 21) as an off-white solid.

¹H NMR (400 MHz, CD₃OD) δ: ppm 7.47-7.40 (m, 5H), 7.00-6.79 (d, 1H), 5.70 (s, 1H), 4.36 (s, 1H), 4.07 (dd, J=8.8, 14.0 Hz, 1H), 3.79 (d, J=10.4 Hz, 1H), 3.62-3.50 (m, 1H), 3.44-3.34 (m, 3H), 2.74 (dq, J=4.8, 8.8 Hz, 1H), 2.39-2.23 (m, 1H), 2.12-1.96 (m, 1H), 1.67-1.55 (m, 2H), 1.11 (s, 6H).

MS obsd. (ESI⁺) [(M+H)⁺]: 590.3.

### Example 22

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,3,3,3-pentafluoro-propanamide

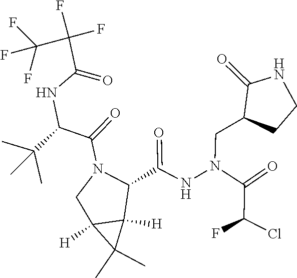 [SMILES: CC(C)(C)[C@H](NC(=O)C(F)(F)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (S)-2-(((benzyloxy)carbonyl)amino)-3,3-dimethylbutanoic acid instead of (2S)-2-(benzyloxycarbonylamino)-4-methyl-pentanoic acid and 2,2,3,3,3-pentafluoropropanoyl 2,2,3,3,3-pentafluoropropanoate instead of TFAA to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,3,3,3-pentafluoro-propanamide (Example 22) as a white solid.

¹H NMR (400 MHz, DMSO-d₆) δ: ppm 11.20 (s, 1H), 9.55 (d, J=8.4 Hz, 1H), 7.76 (s, 1H), 6.85 (d, J=50.4 Hz, 1H), 4.54-4.49 (m, 1H), 4.17 (s, 1H), 4.00-3.90 (m, 1H), 3.84-3.76 (m, 1H), 3.71 (d, J=10.8 Hz, 1H), 3.46 (dd, J=14.0, 4.4 Hz, 1H), 3.25-3.00 (m, 2H), 2.62-2.57 (m, 1H), 2.30-2.10 (m, 1H), 1.85-1.71 (m, 1H), 1.69-1.59 (m, 1H), 1.57-1.53 (m, 1H), 1.05 (s, 3H), 0.98 (s, 9H), 0.88 (s, 3H).

MS obsd. (ESI⁺) [(M+H)⁺]: 620.3.

### Example 23

### N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2,2-trifluoro-acetamide

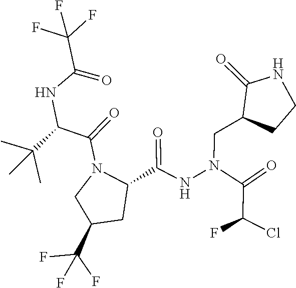 [SMILES: CC(C)(C)[C@H](NC(=O)C(F)(F)F)C(=O)N1C[C@H](C(F)(F)F)C[C@H]1C(=O)NN(C[C@@H]1CCNC1=O)C(=O)[C@H](F)Cl]

The title compound was prepared in analogy to the procedure described for the preparation of Example 1, by (2S,4R)-1-[(2S)-2-amino-3,3-dimethyl-butanoyl]-4-(trifluoromethyl)pyrrolidine-2-carboxylate (Intermediate 15) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2,2-trifluoro-acetamide (Example 23) as a white solid.

¹H NMR (400 MHz, DMSO-d₆) δ: ppm 11.13 (s, 1H), 9.57 (d, J=8.0 Hz, 1H), 7.74 (s, 1H), 6.91 (d, J=50.4 Hz, 1H), 4.56-4.53 (m, 1H), 4.36 (t, J=7.6 Hz, 1H), 4.02-3.97 (m, 2H), 3.73-3.67 (m, 1H), 3.53 (dd, J=4.4, 14.4 Hz, 1H), 3.47-3.38 (m, 1H), 3.18-3.09 (m, 2H), 2.58-2.54 (m, 1H), 2.41-2.35 (m, 1H), 2.27-2.20 (m, 1H), 2.14-2.07 (m, 1H), 1.82-1.74 (m, 1H), 1.00 (s, 9H).

MS obsd. (ESI⁺) [(M+H)⁺]: 598.2.

### Example 24

### N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2,2-trifluoro-acetamide

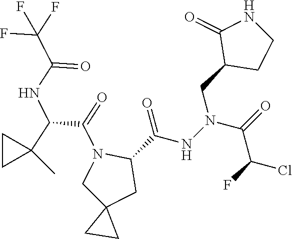 [SMILES: CC1([C@H](NC(=O)C(F)(F)F)C(=O)N2CC3(CC3)C[C@H]2C(=O)NN(C[C@@H]2CCNC2=O)C(=O)[C@H](F)Cl)CC1]

### Step 1: Preparation of benzyl (6S)-5-[(2S)-2-(tert-butoxycarbonylamino)-2-(1-methylcyclopropyl)acetyl]-5-azaspiro[2.4]heptane-6-carboxylate

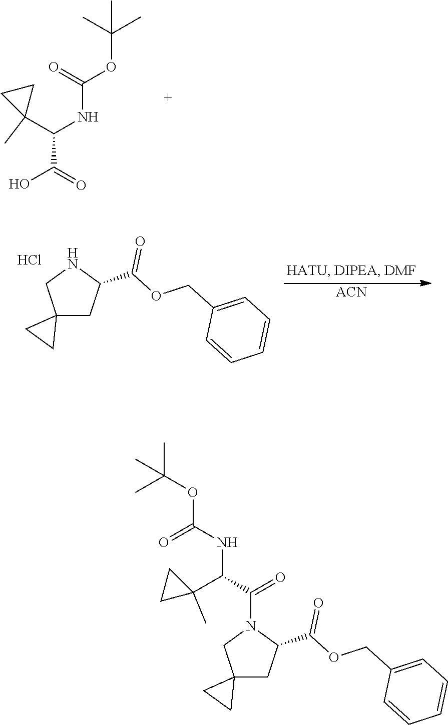 [SMILES: CC(C)(C)OC(=O)N[C@H](C(=O)O)C1(C)CC1 ; Cl ; O=C(OCc1ccccc1)[C@@H]1CC2(CC2)CN1 ; "ACN" ; CC(C)(C)OC(=O)N[C@H](C(=O)N1CC2(CC2)C[C@H]1C(=O)OCc1ccccc1)C1(C)CC1]

To a solution of (2S)-2-(tert-butoxycarbonylamino)-2-(1-methylcyclopropyl)acetic acid (900 mg, 3.93 mmol, Intermediate 16) in DMF (2 mL) and ACN (10 mL) was added HATU (1642 mg, 4.32 mmol) and followed by benzyl (6S)-5-azaspiro[2.4]heptane-6-carboxylate; hydrochloride (2033 mg, 5.89 mmol, Intermediate 17). The mixture was cooled to 0° C. and DIPEA (2537 mg, 19.63 mmol) was added to the mixture dropwise. After addition, the mixture was warmed to 15° C. and stirred at 15° C. for 1 h. The mixture was concentrated in vacuum. The residue was purified by silica gel column eluted with PE/EtOAc=20/1 to 4/1 to afford benzyl (6S)-5-[(2S)-2-(tert-butoxycarbonylamino)-2-(1-methylcyclopropyl)acetyl]-5-azaspiro[2.4]heptane-6-carboxylate (1.1 g) as yellow oil.

MS obsd. (ESI⁺) [(M+H)⁺]: 443.1.

### Step 2: Preparation of (6S)-5-[(2S)-2-(tert-butoxycarbonylamino)-2-(1-methylcyclopropyl)acetyl]-5-azaspiro[2.4]heptane-6-carboxylic acid

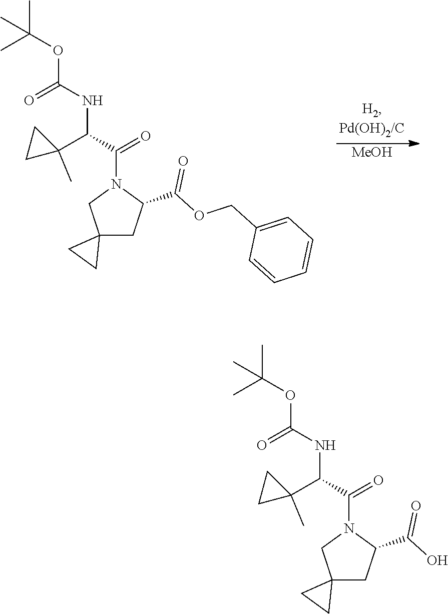 [SMILES: "MeOH" ; CC(C)(C)OC(=O)N[C@H](C(=O)N1CC2(CC2)C[C@H]1C(=O)OCc1ccccc1)C1(C)CC1 ; CC(C)(C)OC(=O)N[C@H](C(=O)N1CC2(CC2)C[C@H]1C(=O)O)C1(C)CC1]

To a solution of benzyl (6S)-5-[(2S)-2-(tert-butoxycarbonylamino)-2-(1-methylcyclopropyl)acetyl]-5-azaspiro[2.4]heptane-6-carboxylate (1.0 g, 2.26 mmol) in methanol (20 mL) was added Pd(OH)₂/C (100 mg). The mixture was degassed under vacuum and purged with hydrogen three times. The resulting suspension was stirred at 25° C. for 1 h under a H₂ balloon. The mixture was filtered and the filtrate was concentrated in vacuum to afford (6S)-5-[(2S)-2-(tert-butoxycarbonylamino)-2-(1-methylcyclopropyl)acetyl]-5-azaspiro[2.4]heptane-6-carboxylic acid (790 mg) as colorless oil.

MS obsd. (ESI⁺) [(M+H)⁺]: 353.1.

### Step 3: Preparation of (6S)-5-[(2S)-2-amino-2-(1-methylcyclopropyl)acetyl]-5-azaspiro[2.4]heptane-6-carboxylic acid; hydrochloride

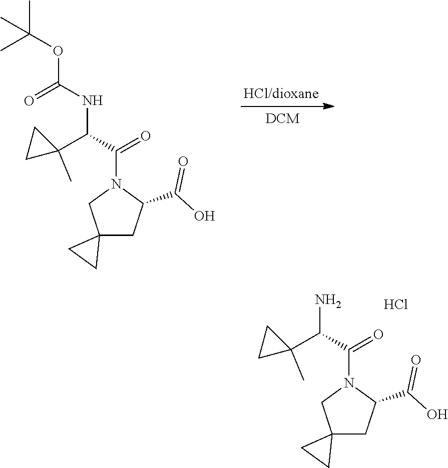 [SMILES: CC(C)(C)OC(=O)N[C@H](C(=O)N1CC2(CC2)C[C@H]1C(=O)O)C1(C)CC1 ; CC1([C@H](N)C(=O)N2CC3(CC3)C[C@H]2C(=O)O)CC1 ; Cl]

To a solution of (6S)-5-[(2S)-2-(tert-butoxycarbonylamino)-2-(1-methylcyclopropyl)acetyl]-5-azaspiro[2.4]heptane-6-carboxylic acid (790 mg, 2.24 mmol) in DCM (5 mL) was added 4 N HCl/dioxane (5.0 mL). The reaction mixture was stirred at 25° C. for 1 h. The mixture was concentrated in vacuum to afford (6S)-5-[(2S)-2-amino-2-(1-methylcyclopropyl)acetyl]-5-azaspiro[2.4]heptane-6-carboxylic acid; hydrochloride (640 mg) as yellow gum.

### Step 4: Preparation of (6S)-5-[(2S)-2-(1-methylcyclopropyl)-2-[(2,2,2-trifluoroacetyl)amino]acetyl]-5-azaspiro[2.4]heptane-6-carboxylic acid

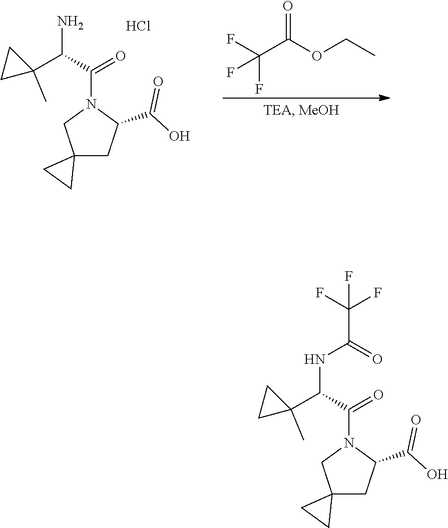

To a solution of (6S)-5-[(2S)-2-amino-2-(1-methylcyclopropyl)acetyl]-5-azaspiro[2.4]heptane-6-carboxylic acid; hydrochloride (640 mg, 2.22 mmol) in methanol (10 mL) was added triethylamine (1.85 mL, 13.3 mmol) dropwise at 0° C., followed by ethyl trifluoroacetate (944 mg, 6.65 mmol). The mixture was stirred at 50° C. for 12 h. The mixture was concentrated in vacuum. The residue was dissolved in EtOAc (100 mL), washed with 1 N HCl (20 mL), brine (20 mL) and dried over Na₂SO₄. After filtration and concentration, (6S)-5-[(2S)-2-(1-methylcyclopropyl)-2-[(2,2,2-trifluoroacetyl)amino]acetyl]-5-azaspiro[2.4]heptane-6-carboxylic acid (770 mg) was obtained as yellow gum.

MS obsd. (ESI⁺) [(M+H)⁺]: 349.1.

### Step 5: Preparation of tert-butyl N-[[(6S)-5-[(2S)-2-(1-methylcyclopropyl)-2-[(2,2,2-trifluoroacetyl)amino]acetyl]-5-azaspiro[2.4]heptane-6-carbonyl]amino]-N-[[(3S)-2-oxopyrrolidin-3-yl]methyl]carbamate

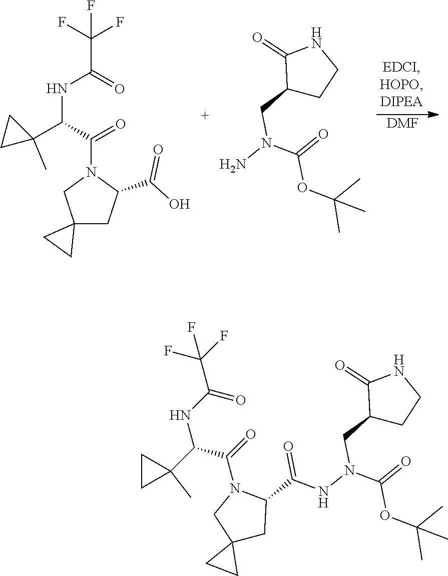

To a solution of (6S)-5-[(2S)-2-(1-methylcyclopropyl)-2-[(2,2,2-trifluoroacetyl)amino]acetyl]-5-azaspiro[2.4]heptane-6-carboxylic acid (760 mg, 2.18 mmol) in DMF (5 mL) was added EDCI (502 mg, 2.62 mmol), DIPEA (846 mg, 6.50 mmol) and HOPO (291 mg, 2.62 mmol). The mixture was stirred at 25° C. for 30 min and then tert-butyl N-amino-N-[[(3S)-2-oxopyrrolidin-3-yl]methyl]carbamate (500 mg, 2.18 mmol) was added into the mixture. The resulting mixture was stirred at 25° C. for 12 h. The mixture was diluted with EtOAc (100 mL) and washed with brine (60 mL), dried over Na₂SO₄, filtered and the filtrate was concentrated in vacuum. The residue was purified by reverse flash eluted with ACN in H₂O (0.1% TFA)=10%˜60% to afford tert-butyl N-[[(6S)-5-[(2S)-2-(1-methylcyclopropyl)-2-[(2,2,2-trifluoroacetyl)amino]acetyl]-5-azaspiro[2.4]heptane-6-carbonyl]amino]-N—[[(3S)-2-oxopyrrolidin-3-yl]methyl]carbamate (340 mg, 0.610 mmol) as a yellow foam.

MS obsd. (ESI⁺) [(M+H)⁺]: 560.3.

### Step 6: Preparation of 2,2,2-trifluoro-N-[(1S)-1-(1-methylcyclopropyl)-2-oxo-2-[(6S)-6-[[[(3S)-2-oxopyrrolidin-3-yl]methylamino]carbamoyl]-5-azaspiro[2.4]heptan-5-yl]ethyl]acetamide

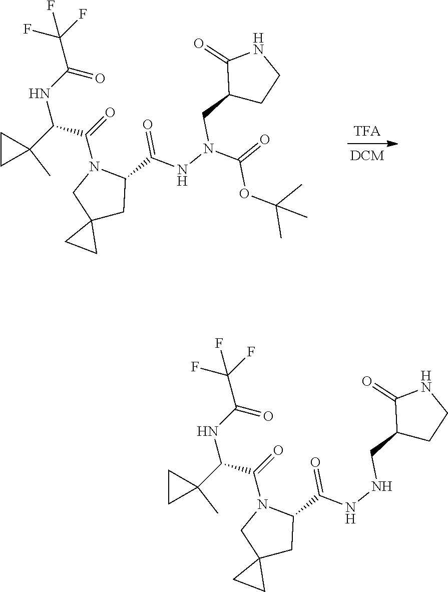 [SMILES: CC(C)(C)OC(=O)N(C[C@@H]1CCNC1=O)NC(=O)[C@@H]1CC2(CC2)CN1C(=O)[C@@H](NC(=O)C(F)(F)F)C1(C)CC1 ; CC1([C@H](NC(=O)C(F)(F)F)C(=O)N2CC3(CC3)C[C@H]2C(=O)NNC[C@@H]2CCNC2=O)CC1]

To a solution of tert-butyl N-[[(6S)-5-[(2S)-2-(1-methylcyclopropyl)-2-[(2,2,2-trifluoroacetyl)amino]acetyl]-5-azaspiro[2.4]heptane-6-carbonyl]amino]-N-[[(3S)-2-oxopyrrolidin-3-yl]methyl]carbamate (330 mg, 0.590 mmol) in DCM (4 mL) was added TFA (2.0 mL). The mixture was stirred at 25° C. for 1 h. The mixture was concentrated in vacuum. The residue dissolved in DCM (50 mL), washed with saturated aqueous solution of NaHCO₃ (10 mL), dried over Na₂SO₄, filtered and the filtrate was concentrated to afford 2,2,2-trifluoro-N-[(1S)-1-(1-methylcyclopropyl)-2-oxo-2-[(6S)-6-[[[(3S)-2-oxopyrrolidin-3-yl]methylamino]carbamoyl]-5-azaspiro[2.4]heptan-5-yl]ethyl]acetamide (180 mg) as a yellow foam.

MS obsd. (ESI⁺) [(M+H)⁺]: 460.2.

### Step 7: Preparation of N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2,2-trifluoro-acetamide (Example 25)

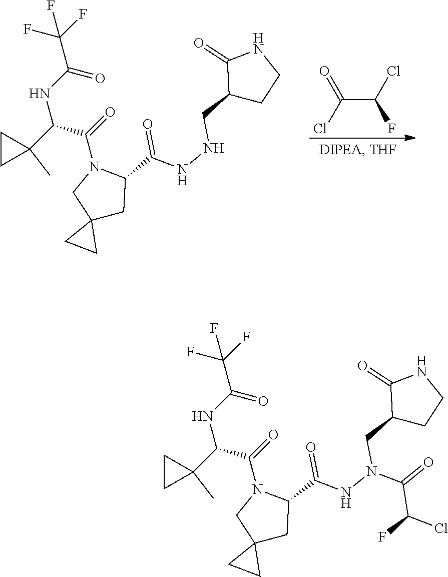

To a solution of 2,2,2-trifluoro-N-[(1S)-1-(1-methylcyclopropyl)-2-oxo-2-[(6S)-6-[[[(3S)-2-oxopyrrolidin-3-yl]methylamino]carbamoyl]-5-azaspiro[2.4]heptan-5-yl]ethyl]acetamide (156 mg, 0.340 mmol) in THF (50 mL) was added DIPEA (439 mg, 3.4 mmol) and (2R)-2-chloro-2-fluoro-acetyl chloride (222 mg, 0.680 mmol) dropwise at 0° C. The mixture was stirred at 0° C. for 30 min. The mixture was adjusted with 4 N HCl/dioxane to pH=5 at 0° C. After concentration at 30° C., the residue was purified by reverse flash (C18, 0.1% FA in water-ACN condition) to afford N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2,2-trifluoro-acetamide (62 mg, Example 24) as a white solid.

¹H NMR (400 MHz, DMSO-d₆) 3: ppm 11.02 (s, 1H), 9.83-9.74 (m, 1H), 7.74 (s, 1H), 6.85 (d, J=10.0 Hz, 1H), 4.52-4.46 (m, 1H), 4.43-4.33 (m, 1H), 3.65-3.47 (m, 1H), 3.64-3.54 (m, 2H), 3.50-3.42 (m, 1H), 3.22-3.09 (m, 2H), 2.63-2.58 (m, 1H), 2.15-2.00 (m, 2H), 1.96-1.88 (m, 1H), 1.84-1.70 (m, 1H), 1.06 (s, 3H), 0.87-0.77 (m, 1H), 0.76-0.55 (m, 5H), 0.36-0.24 (m, 2H).

MS obsd. (ESI⁺) [(M+H)⁺]: 554.2.

### Example 25

### N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2-methyl-propanamide

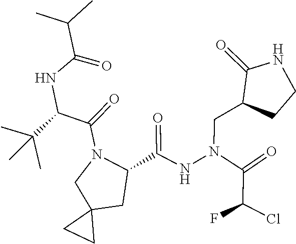 [SMILES: CC(C)C(=O)N[C@H](C(=O)N1CC2(CC2)C[C@H]1C(=O)NN(C[C@@H]1CCNC1=O)C(=O)[C@H](F)Cl)C(C)(C)C]

The title compound was prepared in analogy to the procedure described for the preparation of Example 1, by benzyl (6S)-5-[(2S)-2-amino-3,3-dimethyl-butanoyl]-5-azaspiro[2.4]heptane-6-carboxylate (Intermediate 5) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) and isobutyryl chloride instead of TFAA to afford N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2-methyl-propanamide (Example 25) as a white solid.

¹H NMR (400 MHz, CD₃OD) δ: ppm 7.85 (d, J=8.6 Hz, 1H), 7.11 (d, J=50.4 Hz, 1H), 4.59-4.41 (m, 2H), 3.98 (m, 1H), 3.85 (d, J=9.8 Hz, 1H), 3.73-3.56 (m, 2H), 3.42-3.34 (m, 2H), 2.75-2.57 (m, 2H), 2.36-2.15 (m, 2H), 2.11-1.90 (m, 2H), 1.12 (dd, J=6.8, 16.0 Hz, 6H), 1.05 (s, 9H), 0.78-0.59 (m, 4H).

MS obsd. (ESI⁺) [(M+H)⁺]: 530.3.

### Example 26

### N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]cyclopropanecarboxamide

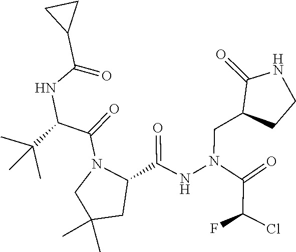 [SMILES: CC1(C)C[C@@H](C(=O)NN(C[C@@H]2CCNC2=O)C(=O)[C@H](F)Cl)N(C(=O)[C@@H](NC(=O)C2CC2)C(C)(C)C)C1]

The title compound was prepared in analogy to the procedure described for the preparation of Example 1, by using benzyl (2S)-1-[(2S)-2-amino-3,3-dimethyl-butanoyl]-4,4-dimethyl-pyrrolidine-2-carboxylate (Intermediate 7) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) and cyclopropanecarbonyl chloride instead of TFAA to afford N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]cyclopropanecarboxamide (Example 26) as a white solid.

¹H NMR (400 MHz, DMSO-d₆) δ: ppm 11.00 (s, 1H), 8.18 (d, J=8.8 Hz, 1H), 7.75 (s, 1H), 7.09 (d, J=50.4 Hz, 1H), 4.39 (d, J=8.4 Hz, 1H), 4.31-4.25 (m, 1H), 3.75-3.64 (m, 2H), 3.42 (dd, J=14.0 Hz, 4.0 Hz, 1H), 3.32-3.27 (m, 2H), 3.22-3.10 (m, 2H), 2.18-2.05 (m, 1H), 1.98-1.85 (m, 2H), 1.80-1.70 (m, 1H), 1.67-1.56 (m, 1H), 1.13 (s, 3H), 0.98 (s, 3H), 0.95 (s, 9H), 0.68-0.58 (m, 4H).

MS obsd. (ESI⁺) [(M+H)⁺]: 530.3.

### Example 27

### N-[(1S)-1-[(2S,4S)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide

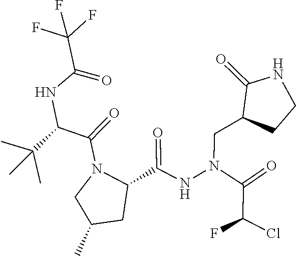 [SMILES: C[C@H]1C[C@@H](C(=O)NN(C[C@@H]2CCNC2=O)C(=O)[C@H](F)Cl)N(C(=O)[C@@H](NC(=O)C(F)(F)F)C(C)(C)C)C1]

The title compound was prepared in analogy to the procedure described for the preparation of Example 1, by using benzyl (2S,4S)-1-[(2S)-2-amino-3,3-dimethyl-butanoyl]-4-methyl-pyrrolidine-2-carboxylate (Intermediate 18) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(2S,4S)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide (Example 27) as a white solid.

¹H NMR (400 MHz, DMSO-d₆) δ: ppm 11.08 (s, 1H), 9.51 (d, J=8.4 Hz, 1H), 7.76 (s, 1H), 7.02 (d, J=50.4 Hz, 1H), 4.58 (d, J=8.4 Hz, 1H), 4.30-4.22 (m, 1H), 4.08-3.95 (m, 1H), 3.80-3.68 (m, 1H), 3.42 (dd, J=14.0 Hz, 4.0 Hz, 1H), 3.23-3.05 (m, 3H), 2.35-2.22 (m, 2H), 2.17-2.05 (m, 1H), 1.82-1.68 (m, 1H), 1.52-1.38 (m, 1H), 1.23-1.14 (m, 1H), 1.07 (d, J=6.0 Hz, 3H), 0.98 (s, 9H).

MS obsd. (ESI⁺) [(M+H)⁺]: 544.1.

### Example 28

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide

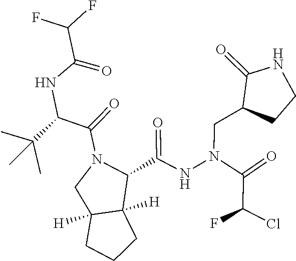 [SMILES: CC(C)(C)[C@H](NC(=O)C(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate (Intermediate 3) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) and (2,2-difluoroacetyl) 2,2-difluoroacetate instead of TFAA to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide (Example 28) as a white solid.

¹H NMR (400 MHz, CDCl₃) δ: ppm 9.95 (br. s, 1H), 7.08-7.04 (m, 1H), 6.78-6.66 (m, 1H), 6.08-5.81 (m, 2H), 4.68 (d, J=9.6 Hz, 1H), 4.47-4.42 (m, 1H), 4.35-4.16 (m, 1H), 4.00 (dd, J=10.4 Hz, 7.6 Hz, 1H), 3.76-3.73 (m, 1H), 3.43-3.39 (m, 2H), 2.88-2.64 (m, 4H), 2.42-2.37 (m, 1H), 1.94-1.66 (m, 6H), 1.49-1.42 (m, 1H), 1.04 (s, 9H).

MS obsd. (ESI⁺) [(M+H)⁺]: 552.2.

### Example 29

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]acetamide

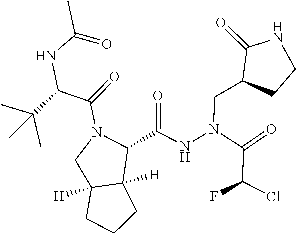 [SMILES: CC(=O)N[C@H](C(=O)N1C[C@@H]2CCC[C@@H]2[C@H]1C(=O)NN(C[C@@H]1CCNC1=O)C(=O)[C@H](F)Cl)C(C)(C)C]

### Step 1: Preparation of methyl (3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carboxylate

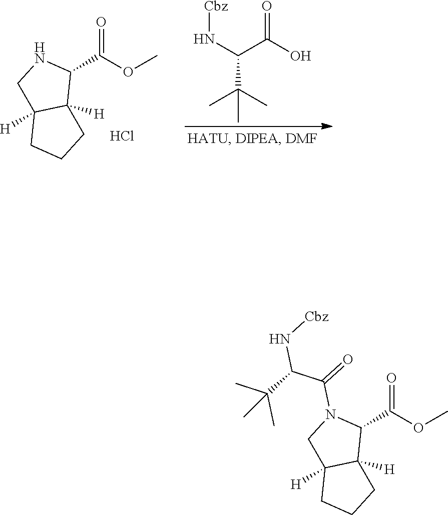 [SMILES: COC(=O)[C@H]1NC[C@@H]2CCC[C@@H]21 ; Cl ; CC(C)(C)[C@H](NC(=O)OCc1ccccc1)C(=O)O ; COC(=O)[C@@H]1[C@H]2CCC[C@H]2CN1C(=O)[C@@H](NC(=O)OCc1ccccc1)C(C)(C)C]

To a solution of Cbz-L-tert-leucine (4.2 g, 15.83 mmol) in DMF (50 mL) was added methyl (3S,3aS,6aR)-1,2,3,3a,4,5,6,6a-octahydrocyclopenta[c]pyrrole-3-carboxylate hydrochloride (4.23 g, 15.83 mmol) and HATU (7.2 g, 19.0 mmol) at 0° C. Then DIPEA (10.2 g, 79.16 mmol) was added into above solution to afford a yellow solution. The mixture was stirred at 25° C. for 12 h. The mixture was diluted with EtOAc (400 mL), washed with brine (60 mL×4), dried over Na₂SO₄, filtered and concentrated in vacuum. The residue was purified by silica gel column eluted with PE to PE/EtOAc=6/1 to afford methyl (3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carboxylate (6.5 g) as yellow gum.

MS obsd. (ESI⁺) [(M+H)⁺]: 417.2.

### Step 2: Preparation of (3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carboxylic acid

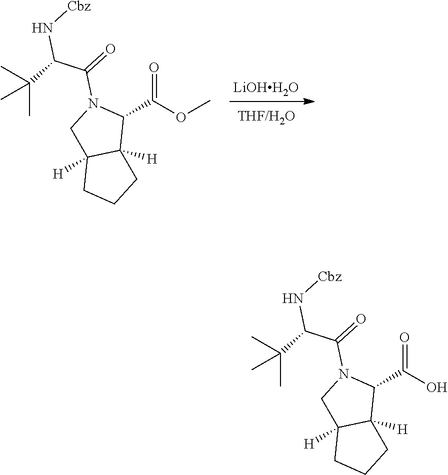

To a solution of methyl (3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carboxylate (6.5 g, 15.61 mmol) in THF (100 mL) was added a solution of LiOH·H₂O (2.0 g, 46.82 mmol) in water (40 mL). The mixture was stirred at 25° C. for 1 h. The mixture was acidified with 1 N HCl to pH=4. The mixture was extracted with EtOAc (100 mL×3). The organic layers were washed with brine (60 mL), dried over Na₂SO₄, filtered and concentrated in vacuum to afford (3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carboxylic acid (5.96 g) as a yellow foam.

MS obsd. (ESI⁺) [(M+H)⁺]: 403.1.

### Step 3: Preparation of tert-butyl N-[[(3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate

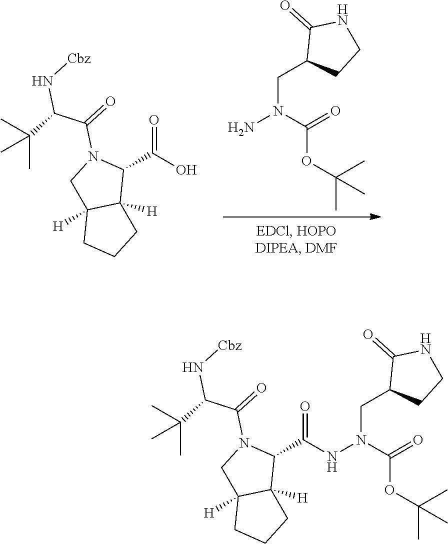 [SMILES: CC(C)(C)[C@H](NC(=O)OCc1ccccc1)C(=O)N1C[C@@H]2CCC[C@@H]2[C@H]1C(=O)O ; CC(C)(C)OC(=O)N(N)C[C@@H]1CCNC1=O ; CC(C)(C)OC(=O)N(C[C@@H]1CCNC1=O)NC(=O)[C@@H]1[C@H]2CCC[C@H]2CN1C(=O)[C@@H](NC(=O)OCc1ccccc1)C(C)(C)C]

To a solution of (3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carboxylic acid (5.9 g, 14.66 mmol) in DMF (60 mL) was added DIPEA (5.7 g, 43.99 mmol), EDCI (3.4 g, 17.6 mmol), HOPO (1.95 g, 17.6 mmol) and tert-butyl N-amino-N-[[(3S)-2-oxopyrrolidin-3-yl]methyl]carbamate (3.4 g, 14.67 mmol, Intermediate 9). The mixture was stirred at 25° C. for 12 h. The mixture was diluted with EtOAc (300 mL), washed with brine (60 mL×4), dried over Na₂SO₄, filtered and concentrated in vacuum. The residue was purified by reverse flash chromatography ([water (0.1% TFA)-ACN]; B %: 50%-55%, 25 min) to afford tert-butyl N-[[(3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (6.2 g) as a yellow foam.

MS obsd. (ESI⁺) [(M+H)⁺]: 614.4.

### Step 4: Preparation of tert-butyl N-[[(3S,3aS,6aR)-2-[(2S)-2-amino-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate

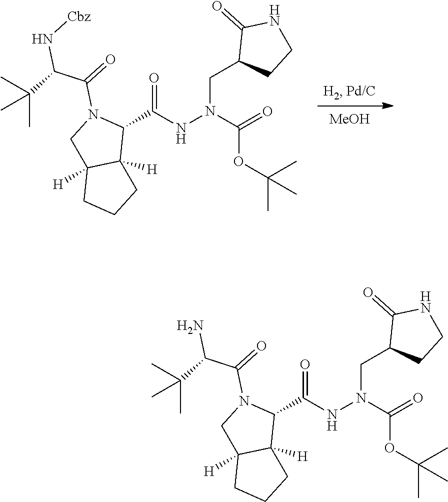

To a solution of tert-butyl N-[[(3S,3aS,6aR)-2-[(2S)-2-(benzyloxycarbonylamino)-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (6.2 g, 10.1 mmol) in methanol (60 mL) was added Pd/C (600 mg, 10% purity). The suspension was degassed under vacuum and purged with hydrogen three times. The resulting mixture was stirred at 25° C. under a H₂ balloon for 2 h. The mixture was filtered and the filter cake was washed with MeOH (50 mL). The filtrate was concentrated in vacuum to afford tert-butyl N-[[(3S,3aS,6aR)-2-[(2S)-2-amino-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (4.5 g) as a yellow foam.

MS obsd. (ESI⁺) [(M+H)⁺]: 480.2.

### Step 5: Preparation of tert-butyl N-[[(1R,2S,5S)-6,6-dimethyl-3-[(2S)-4-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate

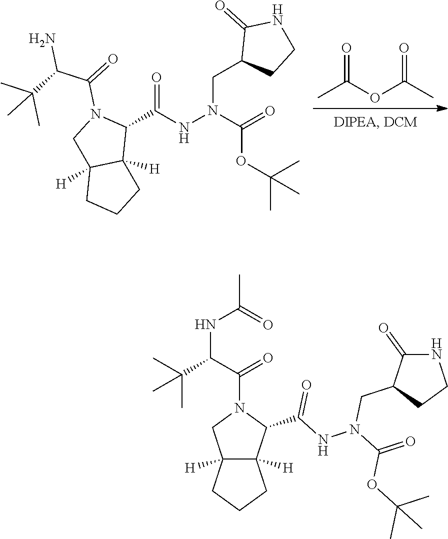

To a solution of tert-butyl N-[[(3S,3aS,6aR)-2-[(2S)-2-amino-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (500 mg, 1.04 mmol) in DCM (6 mL) was added DIPEA (404 mg, 3.13 mmol) and acetic anhydride (159 mg, 1.56 mmol) dropwise at 0° C. The mixture was stirred at 25° C. for 1 h. The mixture was diluted in DCM (30 mL) and washed with 1 N HCl (30 mL). After separation, the aqueous phase was extracted with DCM (30 ml×2). The organic layers were washed with brine (40 ml), dried over Na₂SO₄, filtered and concentrated in vacuum to afford tert-butyl N-[[(1R,2S,5S)-6,6-dimethyl-3-[(2S)-4-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate (520 mg, crude) as yellow oil.

MS obsd. (ESI⁺) [(M+H)⁺]: 522.4.

### Step 6: Preparation of N-[(1S)-1-[(3S,3aS,6aR)-3-[[[(3S)-2-oxopyrrolidin-3-yl]methylamino]carbamoyl]-3,3a,4,5,6,6a-hexahydro-1H-cyclopenta[c]pyrrole-2-carbonyl]-2,2-dimethyl-propyl]acetamide; hydrochloride

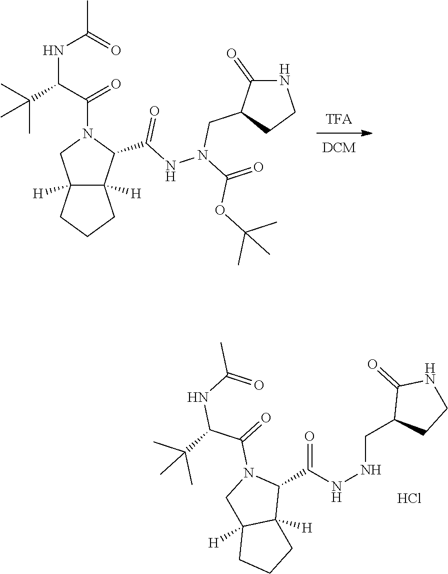 [SMILES: CC(=O)N[C@H](C(=O)N1C[C@@H]2CCC[C@@H]2[C@H]1C(=O)NN(C[C@@H]1CCNC1=O)C(=O)OC(C)(C)C)C(C)(C)C ; CC(=O)N[C@H](C(=O)N1C[C@@H]2CCC[C@@H]2[C@H]1C(=O)NNC[C@@H]1CCNC1=O)C(C)(C)C ; Cl ; "TFA"]

To a solution of tert-butyl N-[[(3S,3aS,6aR)-2-[(2S)-2-acetamido-3,3-dimethyl-butanoyl]-3,3a,4,5,6,6a-hexahydro-TH-cyclopenta[c]pyrrole-3-carbonyl]amino]-N-[[(3S)-2-oxopyrrolidin-3-yl]methyl]carbamate (500 mg, 0.96 mmol) in DCM (5 mL) was added TFA (5 mL). The mixture was stirred at 25° C. for 1 h. The mixture was concentrated in vacuum and the residue was purified by reverse flash chromatography, eluted with ACN in H₂O (0.1% HCl)=0˜60% to afford N-[(1S)-1-[(3S,3aS,6aR)-3-[[[(3S)-2-oxopyrrolidin-3-yl]methylamino]carbamoyl]-3,3a,4,5,6,6a-hexahydro-TH-cyclopenta[c]pyrrole-2-carbonyl]-2,2-dimethyl-propyl]acetamide hydrochloride (350 mg) as a white solid.

MS obsd. (ESI⁺) [(M+H)⁺]: 422.4.

### Step 7: Preparation of N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]acetamide (Example 29)

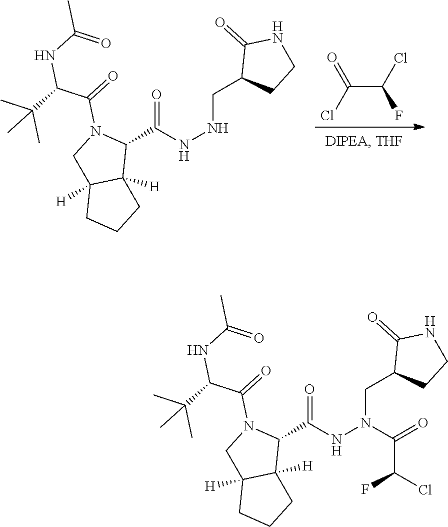

To a solution of N-[(1S)-1-[(3S,3aS,6aR)-3-[[[(3S)-2-oxopyrrolidin-3-yl]methylamino]carbamoyl]-3,3a,4,5,6,6a-hexahydro-1H-cyclopenta[c]pyrrole-2-carbonyl]-2,2-dimethyl-propyl]acetamide; hydrochloride (350 mg, 0.8 mmol) in THF (100 mL) was added DIPEA (986 mg, 7.6 mmol). The mixture was cooled to −5° C. and then (2R)-2-chloro-2-fluoro-acetyl chloride (500 mg, 1.5 mmol) in THF (10 mL) was added dropwise at −5° C. The reaction mixture was stirred at −5° C. for 30 min. The reaction mixture was quenched with MeOH (10 mL) and acidified by 4 N HCl/dioxane to pH=5 at −10° C. Then the mixture was concentrated in vacuum at 30° C. The residue was purified by pre-HPLC (Instrument ACSWH-PREP-NPLC-A Method Column Welch Ultimate XB—SiOH 250*50*10 um Condition Hexane-EtOH Begin B 1 End B 40 Gradient Time (min: 15; 100% B Hold Time (min: 4 FlowRate (ml/min: 100 Injections: 1) to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]acetamide (185 mg, Example 29) as a white solid.

¹H NMR (400 MHz, DMSO-d₆) δ: ppm 11.08 (s, 1H), 7.99 (d, J=8.4 Hz, 1H), 7.78 (s, 1H), 7.02 (d, J=50.4 Hz, 1H), 4.40 (d, J=8.4 Hz, 1H), 3.92 (d, J=6.8 Hz, 1H), 3.90-3.82 (m, 1H), 3.80-3.66 (m, 2H), 3.39 (dd, J=14.0 Hz, 3.6 Hz, 1H), 3.24-3.05 (m, 2H), 2.76-2.68 (m, 1H), 2.64-2.54 (m, 1H), 2.48-2.44 (m, 1H), 2.21-2.05 (m, 1H), 1.88 (s, 3H), 1.86-1.68 (m, 5H), 1.68-1.57 (m, 1H), 1.43-1.29 (m, 1H), 0.93 (s, 9H).

MS obsd. (ESI⁺) [(M+H)⁺]: 516.2.

### Example 30

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-propanamide

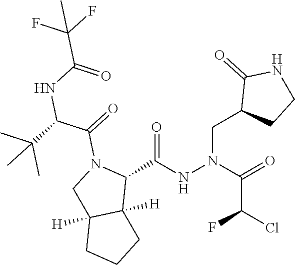 [SMILES: CC(F)(F)C(=O)N[C@H](C(=O)N1C[C@@H]2CCC[C@@H]2[C@H]1C(=O)NN(C[C@@H]1CCNC1=O)C(=O)[C@H](F)Cl)C(C)(C)C]

The title compound was prepared in analogy to the procedure described for the preparation of Example 29, by using 2,2-difluoropropionic acid and T₃P instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-propanamide (Example 30) as a white solid.

¹H NMR (400 MHz, DMSO-d₆) δ: ppm 11.11 (s, 1H), 8.24 (d, J=8.0 Hz, 1H), 7.80 (s, 1H), 6.97 (d, J=50.4 Hz, 1H), 4.57-4.48 (m, 1H), 3.98 (d, J=4.0 Hz, 1H), 3.97-3.83 (m, 1H), 3.78-3.62 (m, 2H), 3.39 (m, 1H), 3.21-3.10 (m, 2H), 2.77-2.71 (m, 1H), 2.61-2.57 (m, 1H), 2.19-2.11 (m, 1H), 1.94-1.55 (m, 10H), 1.42-1.30 (m, 1H), 0.96 (s, 9H).

MS obsd. (ESI⁺) [(M+H)⁺]: 566.2.

### Example 31

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]cyclopropanecarboxamide

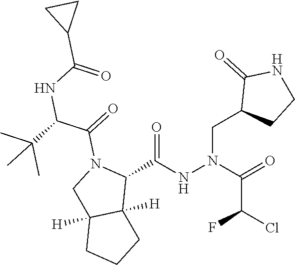 [SMILES: CC(C)(C)[C@H](NC(=O)C1CC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using cyclopropanecarbonyl chloride instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]cyclopropanecarboxamide (Example 31) as a white solid.

¹H NMR (400 MHz, DMSO-d₆) δ: ppm 11.08 (s, 1H), 8.18 (d, J=8.4 Hz, 1H), 7.77 (s, 1H), 7.05 (d, J=50.4 Hz, 1H), 4.43 (d, J=8.8 Hz, 1H), 3.93 (d, J=6.4 Hz, 1H), 3.89-3.80 (m, 1H), 3.78-3.69 (m, 2H), 3.42-3.36 (m, 1H), 3.24-3.06 (m, 2H), 2.75-2.69 (m, 1H), 2.28-2.01 (m, 1H), 1.94-1.81 (m, 2H), 1.80-1.69 (m, 4H), 1.68-1.58 (m, 1H), 1.37-1.22 (m, 1H), 1.02-0.95 (m, 11H), 0.70-0.55 (m, 4H).

MS obsd. (ESI⁺) [(M+H)⁺]: 542.3.

### Example 32

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]propanamide

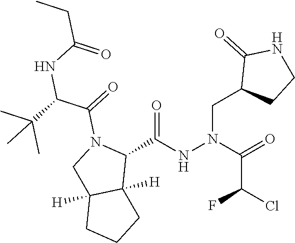 [SMILES: CCC(=O)N[C@H](C(=O)N1C[C@@H]2CCC[C@@H]2[C@H]1C(=O)NN(C[C@@H]1CCNC1=O)C(=O)[C@H](F)Cl)C(C)(C)C]

The title compound was prepared in analogy to the procedure described for the preparation of Example 29, by using propionyl chloride instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]propanamide (Example 32) as a white solid.

¹H NMR (400 MHz, DMSO-d₆) δ: ppm 11.07 (s, 1H), 7.89 (d, J=8.4 Hz, 1H), 7.78 (s, 1H), 7.05 (d, J=50.4 Hz, 1H), 4.42 (d, J=8.8 Hz, 1H), 3.92 (d, J=6.4 Hz, 1H), 3.89-3.81 (m, 1H), 3.79-3.67 (m, 2H), 3.39 (dd, J=14.0 Hz, 4.0 Hz, 1H), 3.22-3.10 (m, 2H), 2.77-2.68 (m, 1H), 2.59-2.53 (m, 2H), 2.29-2.08 (m, 3H), 1.90-1.70 (m, 5H), 1.68-1.56 (m, 1H), 1.40-1.30 (m, 1H), 0.97 (t, J=7.6 Hz, 3H), 0.93 (s, 9H).

MS obsd. (ESI⁺) [(M+H)⁺]: 530.3.

### Example 33

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-methyl-propanamide

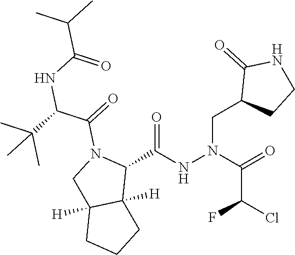 [SMILES: CC(C)C(=O)N[C@H](C(=O)N1C[C@@H]2CCC[C@@H]2[C@H]1C(=O)NN(C[C@@H]1CCNC1=O)C(=O)[C@H](F)Cl)C(C)(C)C]

The title compound was prepared in analogy to the procedure described for the preparation of Example 29, by using isobutyryl chloride instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-methyl-propanamide (Example 33) as a white solid.

¹H NMR (400 MHz, DMSO-d₆) δ: ppm 11.09 (s, 1H), 7.85 (d, J=8.8 Hz, 1H), 7.79 (s, 1H), 7.03 (d, J=52.0 Hz, 1H), 4.43 (d, J=8.0 Hz, 1H), 3.93 (d, J=8.0 Hz, 1H), 3.89-3.81 (m, 1H), 3.77-3.67 (m, 2H), 3.38 (dd, J=16.0, 4.0 Hz, 1H), 3.21-3.10 (m, 2H), 2.79-2.56 (m, 3H), 2.19-2.10 (m, 1H), 1.87-1.80 (m, 2H), 1.75-1.60 (m, 4H), 1.67-1.50 (m, 1H), 1.20-1.40 (m, 1H), 0.99 (d, J=4.0 Hz, 3H), 0.95-0.90 (m, 12H).

MS obsd. (ESI⁺) [(M+H)⁺]: 544.3.

### Example 34

### Rac-2,2-difluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]cyclopropanecarboxamide

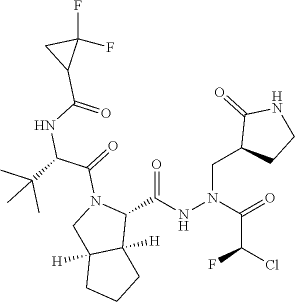 [SMILES: CC(C)(C)[C@H](NC(=O)C1CC1(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using 2,2-difluorocyclopropanecarboxylic acid and T₃P instead of acetic anhydride to afford rac-2,2-difluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]cyclopropanecarboxamide (Example 34) as a white solid.

¹H NMR (400 MHz, DMSO-d₆) δ: ppm 11.08 (d, J=8.0 Hz, 1H), 8.60-8.45 (m, 1H), 7.87 (s, 1H), 7.03 (d, J=52.0 Hz, 1H), 4.48 (d, J=8.4 Hz, 1H), 3.93 (d, J=8.0 Hz, 1H), 3.89-3.81 (m, 1H), 3.77-3.67 (m, 2H), 3.38 (dd, J=14.0, 3.6 Hz, 1H), 3.21-3.10 (m, 2H), 2.95-2.83 (m, 1H), 2.74-2.69 (m, 1H), 2.58-2.56 (m, 2H), 2.19-2.10 (m, 1H), 1.94-1.82 (m, 3H), 1.80-1.67 (m, 4H), 1.67-1.55 (m, 1H), 1.40-1.25 (m, 1H), 0.95 (s, 9H).

MS obsd. (ESI⁺) [(M+H)⁺]: 578.3.

### Example 35

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-fluoro-2-methyl-propanamide

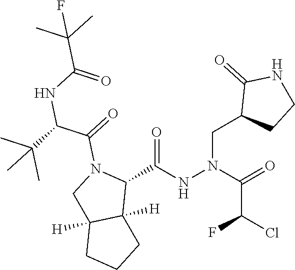 [SMILES: CC(C)(F)C(=O)N[C@H](C(=O)N1C[C@@H]2CCC[C@@H]2[C@H]1C(=O)NN(C[C@@H]1CCNC1=O)C(=O)[C@H](F)Cl)C(C)(C)C]

The title compound was prepared in analogy to the procedure described for the preparation of Example 29, by using 2-fluoroisobutyric acid and T₃P instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-fluoro-2-methyl-propanamide (Example 35) as a white solid.

¹H NMR (400 MHz, DMSO-d₆) δ: ppm 11.11 (s, 1H), 7.77 (s, 1H), 7.18 (dd, J=8.4 Hz, 4.0 Hz, 1H), 6.95 (d, J=50.8 Hz, 1H), 4.46 (d, J=8.4 Hz, 1H), 3.98 (d, J=6.0 Hz, 1H), 3.89 (dd, J=10.0 Hz, 7.2 Hz, 1H), 3.73 (dd, J=13.6 Hz, 3.2 Hz, 1H), 3.65 (dd, J=10.8 Hz, 3.2 Hz, 1H), 3.40 (dd, J=14.0 Hz, 4.0 Hz, 1H), 3.22-3.10 (m, 2H), 2.78-2.69 (m, 1H), 2.60-2.54 (m, 1H), 2.53-2.51 (m, 1H), 2.21-2.10 (m, 1H), 1.89-1.69 (m, 5H), 1.67-1.60 (m, 1H), 1.55-1.43 (m, 6H), 1.40-1.31 (m, 1H), 0.94 (s, 9H).

MS obsd. (ESI⁺) [(M+H)⁺]: 562.2.

### Example 36

### Rac-2-fluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]propanamide

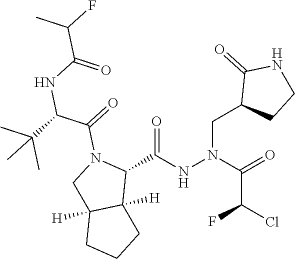 [SMILES: CC(F)C(=O)N[C@H](C(=O)N1C[C@@H]2CCC[C@@H]2[C@H]1C(=O)NN(C[C@@H]1CCNC1=O)C(=O)[C@H](F)Cl)C(C)(C)C]

The title compound was prepared in analogy to the procedure described for the preparation of Example 29, by using 2-fluoropropionic acid and T₃P instead of acetic anhydride to afford Rac-2-fluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]propanamide (Example 36) as a white solid.

¹H NMR (400 MHz, DMSO-d₆) δ: ppm 11.10 (d, J=4.4 Hz, 1H), 8.11-7.82 (m, 1H), 7.72 (s, 1H), 7.07-6.91 (m, 1H), 5.29-5.04 (m, 1H), 4.51-4.40 (m, 1H), 3.98-3.93 (m, 1H), 3.92-3.85 (m, 1H), 3.79-3.64 (m, 2H), 3.44-3.35 (m, 1H), 3.22-3.10 (m, 2H), 2.79-2.69 (m, 1H), 2.60-2.54 (m, 1H), 2.54-2.51 (m, 1H), 2.22-2.10 (m, 1H), 1.92-1.70 (m, 5H), 1.67-1.58 (m, 1H), 1.45-1.31 (m, 4H), 0.94 (d, J=5.6 Hz, 9H).

MS obsd. (ESI⁺) [(M+H)⁺]: 548.2.

### Example 37

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]cyclopropanecarboxamide

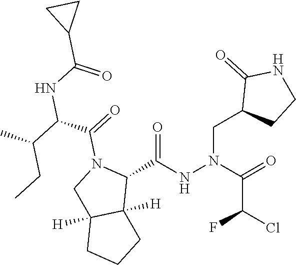 [SMILES: CC[C@H](C)[C@H](NC(=O)C1CC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid (TCI, CAS number: 3160-59-6) instead of Cbz-L-tert-leucine and cyclopropanecarbonyl chloride instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]cyclopropanecarboxamide (Example 37) as a white solid.

¹H NMR (400 MHz, DMSO-d₆) δ: ppm 11.05 (s, 1H), 8.45 (d, J=8.4 Hz, 1H), 7.78 (s, 1H), 7.00 (d, J=50.4 Hz, 1H), 4.26 (t, J=8.8 Hz, 1H), 3.94 (d, J=6.0 Hz, 1H), 3.81-3.70 (m, 3H), 3.39 (dd, J=13.6 Hz, 3.2 Hz, 1H), 3.23-3.10 (m, 2H), 2.74-2.70 (m, 1H), 2.57-2.54 (m, 1H), 2.20-2.09 (m, 1H), 1.93-1.57 (m, 9H), 1.54-1.45 (m, 1H), 1.40-1.30 (m, 1H), 1.18-1.04 (m, 1H), 0.91-0.75 (m, 6H), 0.72-0.56 (m, 4H).

MS obsd. (ESI⁺) [(M+H)⁺]: 542.3.

### Example 38

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]propanamide

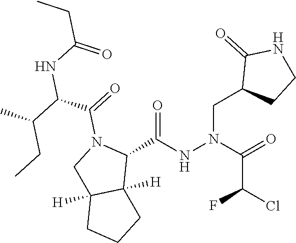 [SMILES: CCC(=O)N[C@H](C(=O)N1C[C@@H]2CCC[C@@H]2[C@H]1C(=O)NN(C[C@@H]1CCNC1=O)C(=O)[C@H](F)Cl)[C@@H](C)CC]

The title compound was prepared in analogy to the procedure described for the preparation of Example 29, by using (2S,3S)-2-(benzyloxycarbonylamino)-3-methyl-pentanoic acid instead of Cbz-L-tert-leucine and propionyl chloride instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]propanamide (Example 38) as a white solid.

¹H NMR (400 MHz, DMSO-d₆) δ: ppm 11.05 (s, 1H), 8.22-8.17 (m, 1H), 7.69 (s, 1H), 6.99 (d, J=50.4 Hz, 1H), 4.31-4.18 (m, 1H), 3.94 (d, J=6.0 Hz, 1H), 3.88-3.79 (m, 1H), 3.77-3.68 (m, 1H), 3.41-3.35 (m, 1H), 3.22-3.10 (m, 2H), 2.80-2.69 (m, 2H), 2.18-2.02 (m, 3H), 1.91-1.62 (m, 8H), 1.55-1.41 (m, 1H), 1.41-1.32 (m, 1H), 1.31-1.21 (m, 1H), 1.13-1.04 (m, 1H), 1.01-0.94 (m, 3H), 0.88-0.78 (m, 6H).

MS obsd. (ESI⁺) [(M+H)⁺]: 530.2.

### Example 39

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide

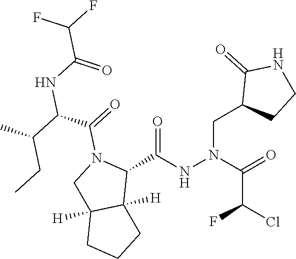 [SMILES: CC[C@H](C)[C@H](NC(=O)C(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine and (2,2-difluoroacetyl) 2,2-difluoroacetate instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide (Example 39) as a white solid.

¹H NMR (400 MHz, DMSO-d₆) δ: ppm 11.08 (s, 1H), 9.23 (d, J=8.0 Hz, 1H), 7.78 (s, 1H), 6.95 (d, J=50.4 Hz, 1H), 6.24 (t, J=53.6 Hz, 1H), 4.38-4.28 (m, 1H), 3.97 (d, J=6.0 Hz, 1H), 3.90-3.70 (m, 3H), 3.40 (dd, J=4.0, 14.0 Hz, 1H), 3.21-3.10 (m, 2H), 2.79-2.71 (m, 1H), 2.62-2.57 (m, 1H), 2.21-2.11 (m, 1H), 1.91-1.69 (m, 6H), 1.67-1.58 (m, 1H), 1.52-1.47 (m, 1H), 1.46-1.34 (m, 2H), 1.15-1.03 (m, 1H), 0.86 (d, J=6.8 Hz, 3H), 0.81 (t, J=7.2 Hz, 3H).

MS obsd. (ESI⁺) [(M+H)⁺]: 552.2.

### Example 40

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide

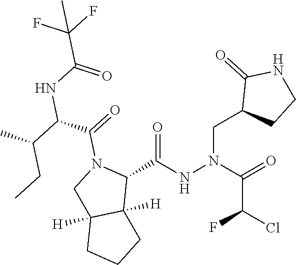 [SMILES: CC[C@H](C)[C@H](NC(=O)C(C)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, 2,2-difluoropropionic acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide (Example 40) as a white solid.

¹H NMR (400 MHz, DMSO-d₆) δ: ppm 11.06 (s, 1H), 8.98 (d, J=7.2 Hz, 1H), 7.78 (s, 1H), 6.95 (d, J=50.4 Hz, 1H), 4.29-4.20 (m, 1H), 3.97 (d, J=6.0 Hz, 1H), 3.84 (d, J=4.8 Hz, 2H), 3.78-3.71 (m, 2H), 3.40 (dd, J=14.0 Hz, 4.0 Hz, 1H), 3.22-3.11 (m, 2H), 2.79-2.70 (m, 1H), 2.62-2.56 (m, 1H), 2.20-2.11 (m, 1H), 1.98-1.66 (m, 10H), 1.65-1.57 (m, 1H), 1.51-1.43 (m, 1H), 1.42-1.34 (m, 1H), 1.14-1.03 (m, 1H), 0.85 (d, J=6.8 Hz, 3H), 0.80 (t, J=7.2 Hz, 2H).

MS obsd. (ESI⁺) [(M+H)⁺]: 566.1.

### Example 41

### Rac-2,2-difluoro-N-[(1S,2S)-2-methyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]cyclopropanecarboxamide

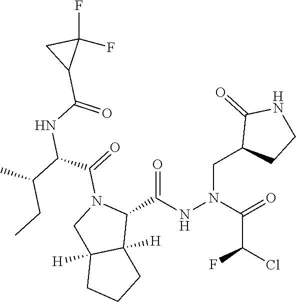 [SMILES: CC[C@H](C)[C@H](NC(=O)C1CC1(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, 2,2-difluorocyclopropanecarboxylic acid and T₃P instead of acetic anhydride to afford rac-2,2-difluoro-N-[(1S,2S)-2-methyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]cyclopropanecarboxamide (Example 41) as a white solid.

¹H NMR (400 MHz, DMSO-d₆) δ: ppm 11.08-11.04 (m, 1H), 8.78-8.68 (m, 1H), 7.77 (s, 1H), 6.93 (d, J=50.4 Hz, 1H), 4.41-4.13 (m, 1H), 4.06-3.85 (m, 1H), 3.86-3.65 (m, 3H), 3.45-3.40 (m, 1H), 3.21-3.12 (m, 2H), 2.62-2.58 (m, 1H), 2.20-2.11 (m, 2H), 1.89-1.69 (m, 8H), 1.63-1.45 (m, 2H), 1.41-1.29 (m, 1H), 1.27-1.18 (m, 1H), 1.15-1.04 (m, 2H), 0.91-0.72 (m, 6H).

MS obsd. (ESI⁺) [(M+H)⁺]: 578.3.

### Example 42

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-fluoro-2-methyl-propanamide

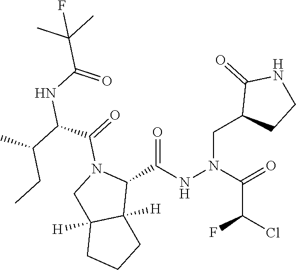 [SMILES: CC[C@H](C)[C@H](NC(=O)C(C)(C)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, 2-fluoroisobutyric acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-fluoro-2-methyl-propanamide (Example 42) as a white solid.

¹H NMR (400 MHz, DMSO-d₆) δ: ppm 11.09 (s, 1H), 8.10 (d, J=1.2 Hz, 1H), 7.79 (s, 1H), 6.92 (d, J=50.4 Hz, 1H), 4.26 (t, J=8.8 Hz, 1H), 4.15-4.05 (m, 1H), 3.96 (d, J=6.0 Hz, 1H), 3.86-3.77 (m, 1H), 3.76-3.69 (m, 1H), 3.39 (dd, J=14.0 Hz, 3.6 Hz, 1H), 3.22-3.10 (m, 2H), 2.78-2.69 (m, 1H), 2.65-2.56 (m, 1H), 2.20-2.10 (m, 1H), 1.96-1.60 (m, 8H), 1.51-1.39 (m, 7H), 1.38-1.31 (m, 1H) 1.10-1.02 (m, 1H) 0.89-0.75 (m, 6H).

MS obsd. (ESI⁺) [(M+H)⁺]: 562.3.

### Example 43

### Rac-2-fluoro-N-[(1S,2S)-2-methyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]propanamide

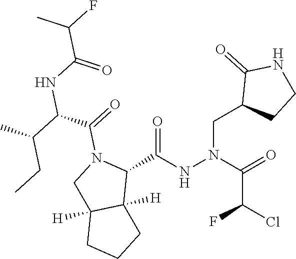 [SMILES: CC[C@H](C)[C@H](NC(=O)C(C)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, 2-fluoropropionic acid and T₃P instead of acetic anhydride to afford rac-2-fluoro-N-[(1S,2S)-2-methyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]propanamide (Example 43) as a white solid.

¹H NMR (400 MHz, DMSO-d₆) δ: ppm 11.08 (d, J=6.0 Hz, 1H), 8.43 (dd, J=34.4 Hz, 8.0 Hz, 1H), 7.80 (s, 1H), 6.96 (dd, J=50.4 Hz, 6.0 Hz, 1H), 5.15-4.91 (m, 1H), 4.28 (q, J=8.4 Hz, 1H), 3.95 (t, J=6.4 Hz, 1H), 3.85-3.69 (m, 3H), 3.39 (dd, J=8.8 Hz, 3.6 Hz, 1H), 3.21-3.10 (m, 2H), 2.76-2.70 (m, 1H), 2.60-2.56 (m, 1H), 2.20-2.08 (m, 1H), 1.90-1.60 (m, 8H), 1.50-1.44 (m, 1H), 1.43-1.25 (m, 4H) 1.15-1.00 (m, 1H), 0.89-0.75 (m, 6H).

MS obsd. (ESI⁺) [(M+H)⁺]: 548.2.

### Example 44

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dimethyl-propanamide

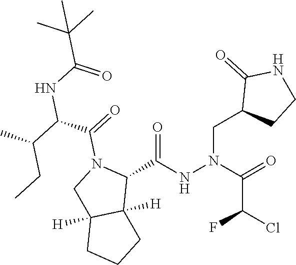 [SMILES: CC[C@H](C)[C@H](NC(=O)C(C)(C)C)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine and pivaloyl chloride instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dimethyl-propanamide (Example 44) as a white solid.

¹H NMR (400 MHz, DMSO-d₆) δ: ppm 11.06 (s, 1H), 7.80 (s, 1H), 7.65 (d, J=8.0 Hz, 1H), 6.93 (d, J=50.4 Hz, 1H), 4.23 (t, J=9.2 Hz, 1H), 3.98-3.86 (m, 2H), 3.81-3.68 (m, 2H), 3.41 (d, J=3.8 Hz, 1H), 3.21-3.10 (m, 2H), 2.75-2.71 (m, 1H), 2.56-2.54 (m, 1H), 2.19-2.10 (m, 1H), 1.93-1.60 (m, 8H), 1.42-1.52 (m, 1H), 1.31-1.40 (m, 1H), 1.09 (s, 9H), 1.06-1.01 (m, 1H), 0.76-0.84 (m, 6H).

MS obsd. (ESI⁺) [(M+H)⁺]: 558.2.

### Example 45

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3,3-trifluoro-propanamide

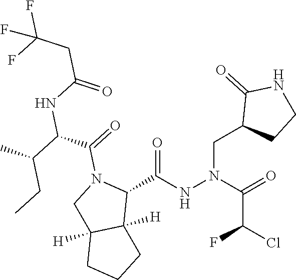 [SMILES: CC[C@H](C)[C@H](NC(=O)CC(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, 3,3,3-trifluoropropionic acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3,3-trifluoro-propanamide (Example 45) as a white solid.

¹H NMR (400 MHz, DMSO-d₆) δ: ppm 11.08 (s, 1H), 8.74-8.56 (m, 1H), 7.78 (s, 1H), 6.93 (d, J=50.4 Hz, 1H), 4.33 (t, J=8.8 Hz, 1H), 3.95 (d, J=6.0 Hz, 1H), 3.67-3.88 (m, 3H), 3.43-3.36 (m, 1H), 3.30-3.22 (m, 2H), 3.22-3.10 (m, 2H), 2.80-2.70 (m, 1H), 2.62-2.54 (m, 2H), 2.25-2.09 (m, 1H), 1.92-1.59 (m, 7H), 1.51-1.31 (m, 2H), 1.19-1.02 (m, 1H), 0.88-0.76 (m, 6H).

MS obsd. (ESI⁺) [(M+H)⁺]: 584.1.

### Example 46

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]butanamide

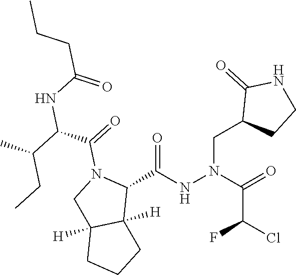 [SMILES: CCCC(=O)N[C@H](C(=O)N1C[C@@H]2CCC[C@@H]2[C@H]1C(=O)NN(C[C@@H]1CCNC1=O)C(=O)[C@H](F)Cl)[C@@H](C)CC]

The title compound was prepared in analogy to the procedure described for the preparation of Example 29, by using (2S,3S)-2-(benzyloxycarbonylamino)-3-methyl-pentanoic acid instead of Cbz-L-tert-leucine, butyric acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]butanamide (Example 46) as a white solid.

¹H NMR (400 MHz, DMSO-d₆) δ: ppm 11.05 (s, 1H), 8.24-8.08 (m, 1H), 7.78 (s, 1H), 6.94 (d, J=50.4 Hz, 1H), 4.25 (t, J=9.0 Hz, 1H), 4.07-3.66 (m, 4H), 3.43-3.35 (m, 1H), 3.21-3.09 (m, 2H), 2.78-2.68 (m, 1H), 2.60-2.51 (m, 2H), 2.19-2.00 (m, 3H), 1.89-1.58 (m, 7H), 1.54-1.42 (m, 3H), 1.41-1.30 (m, 1H), 1.16-1.01 (m, 1H), 0.86-0.75 (m, 9H).

MS obsd. (ESI⁺) [(M+H)⁺]: 544.1.

### Example 47

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-methyl-propanamide

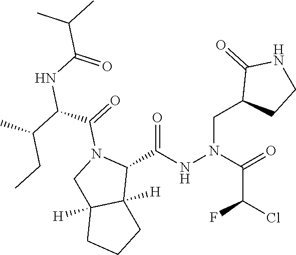 [SMILES: CC[C@H](C)[C@H](NC(=O)C(C)C)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine and isobutyryl chloride instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-methyl-propanamide (Example 47) as a white solid.

¹H NMR (400 MHz, DMSO-d₆) δ: ppm 11.04 (s, 1H), 8.10 (d, J=8.0 Hz, 1H), 7.78 (s, 1H), 6.98 (d, J=50.4 Hz, 1H), 4.30-4.20 (m, 1H), 4.09-3.92 (m, 1H), 3.88-3.68 (m, 3H), 3.43-3.36 (m, 1H), 3.23-3.09 (m, 2H), 2.77-2.69 (m, 1H), 2.60-2.55 (m, 2H), 2.57-2.45 (m, 1H), 2.20-2.10 (m, 1H), 1.91-1.78 (m, 2H), 1.77-1.61 (m, 5H), 1.51-1.43 (m, 1H), 1.40-1.30 (m, 1H), 1.14-1.04 (m, 1H), 0.99 (d, J=6.8 Hz, 3H), 0.94 (d, J=6.8 Hz, 3H), 0.84-0.76 (m, 6H).

MS obsd. (ESI⁺) [(M+H)⁺]: 544.4.

### Example 48

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide

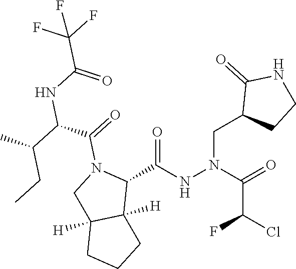 [SMILES: CC[C@H](C)[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine and TFAA instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide (Example 48) as a white solid.

¹H NMR (400 MHz, DMSO-d₆) δ: ppm 11.09 (s, 1H), 9.94 (d, J=7.6 Hz, 1H), 7.78 (s, 1H), 6.93 (d, J=50.4 Hz, 1H), 4.32-4.24 (m, 1H), 3.99 (d, J=6.0 Hz, 1H), 3.91-3.73 (m, 3H), 3.40 (dd, J=13.6 Hz, 4.0 Hz, 1H), 3.20-3.10 (m, 2H), 2.81-2.72 (m, 1H), 2.63-2.57 (m, 1H), 2.21-2.11 (m, 1H), 1.98-1.83 (m, 3H), 1.79-1.71 (m, 3H), 1.68-1.61 (m, 1H), 1.53-1.44 (m, 1H), 1.43-1.35 (m, 1H), 1.20 (t, J=6.5 Hz, 1H), 1.16-1.07 (m, 1H), 0.87 (d, J=6.8 Hz, 3H), 1.81 (t, J=7.2 Hz, 3H).

MS obsd. (ESI⁺) [(M+H)⁺]: 570.2.

### Example 49

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dichloro-acetamide

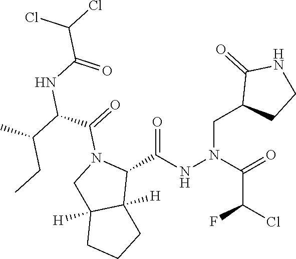 [SMILES: CC[C@H](C)[C@H](NC(=O)C(Cl)Cl)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, dichloroacetic acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dichloro-acetamide (Example 49) as a white solid.

¹H NMR (400 MHz, DMSO-d₆) δ: ppm 11.10 (s, 1H), 9.03-8.83 (m, 1H), 7.75 (s, 1H), 6.93 (d, J=50.8 Hz, 1H), 6.49 (s, 1H), 4.32 (t, J=8.5 Hz, 1H), 3.99 (d, J=6.0 Hz, 1H), 3.90-3.82 (m, 1H), 3.80-3.66 (m, 2H), 3.43-3.35 (m, 1H), 3.23-3.10 (m, 2H), 2.78-2.74 (m, 1H), 2.58-2.57 (m, 1H), 2.21-2.10 (m, 1H), 1.92-1.56 (m, 8H), 1.52-1.34 (m, 2H), 1.16-1.03 (m, 1H), 0.90-0.77 (m, 6H).

MS obsd. (ESI⁺) [(M+H)⁺]: 584.0

### Example 50

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,3,3,3-pentafluoro-propanamide

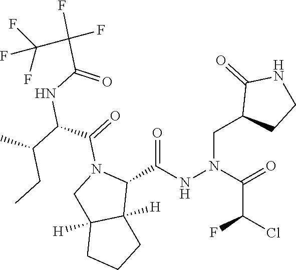 [SMILES: CC[C@H](C)[C@H](NC(=O)C(F)(F)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine and 2,2,3,3,3-pentafluoropropanoyl 2,2,3,3,3-pentafluoropropanoate instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,3,3,3-pentafluoro-propanamide (Example 50) as a white solid.

¹H NMR (400 MHz, DMSO-d₆) δ: ppm 11.10 (s, 1H), 10.04 (d, J=7.2 Hz, 1H), 7.78 (s, 1H), 6.93 (d, J=50.8 Hz, 1H), 4.38-4.27 (m, 1H), 4.12-3.96 (m, 1H), 3.92-3.69 (m, 3H), 3.44-3.36 (m, 1H), 3.22-3.05 (m, 2H), 2.80-2.72 (m, 1H), 2.62-2.57 (m, 2H), 2.21-2.08 (m, 1H), 2.04-1.92 (m, 1H), 1.91-1.57 (m, 6H), 1.52-1.31 (m, 2H), 1.18-1.04 (m, 1H), 0.96-0.76 (m, 6H).

MS obsd. (ESI⁺) [(M+H)⁺]: 620.3.

### Example 51

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide

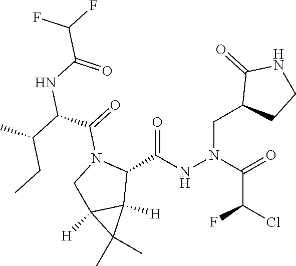 [SMILES: CC[C@H](C)[C@H](NC(=O)C(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid and (2,2-difluoroacetyl) 2,2-difluoroacetate instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide (Example 51) as a white solid.

¹H NMR (400 MHz, DMSO-d₆) δ: ppm 11.15 (s, 1H), 9.26 (d, J=8.0 Hz, 1H), 7.76 (s, 1H), 6.85 (d, J=50.4 Hz, 1H), 6.21 (t, J=53.6 Hz, 1H), 4.27-4.17 (m, 1H), 4.15 (s, 1H), 3.95-3.75 (m, 3H), 3.50-3.42 (m, 1H), 3.23-3.10 (m, 2H), 2.64-2.60 (m, 1H), 2.21-2.08 (m, 1H), 1.88-1.70 (m, 2H), 1.68-1.60 (m, 1H), 1.57-1.44 (m, 2H), 1.18-1.15 (m, 1H), 1.06 (s, 3H), 0.92 (s, 3H), 0.88-0.78 (m, 6H).

MS obsd. (ESI⁺) [(M+H)⁺]: 552.3.

### Example 52

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-dimethyl-propanamide

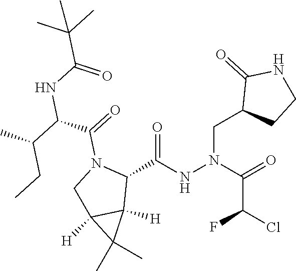 [SMILES: CC[C@H](C)[C@H](NC(=O)C(C)(C)C)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid and pivaloyl chloride instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-dimethyl-propanamide (Example 52) as a white solid.

¹H NMR (400 MHz, DMSO-d₆) δ: ppm 11.13 (s, 1H), 7.76 (s, 1H), 7.67 (d, J=8.4 Hz, 1H), 6.90 (d, J=50.4 Hz, 1H), 4.27-4.18 (m, 1H), 4.13 (s, 1H), 4.00 (d, J=10.4 Hz, 1H), 3.87-3.75 (m, 2H), 3.48-3.41 (dd, J=14.0 Hz, 4.4 Hz, 1H), 3.22-3.10 (m, 2H), 2.63-2.55 (m, 1H), 2.27-2.10 (m, 1H), 1.98-1.84 (m, 1H), 1.83-1.71 (m, 1H), 1.64-1.54 (m, 1H), 1.52-1.39 (m, 2H), 1.10-1.02 (m, 13H), 0.88 (s, 3H), 0.82-0.75 (m, 6H).

MS obsd. (ESI⁺) [(M+H)⁺]: 558.4.

### Example 53

### Rac-2,2-difluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide

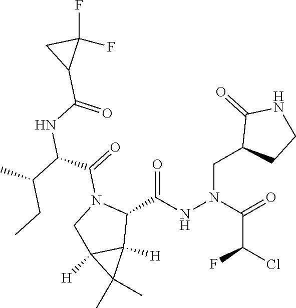 [SMILES: CC[C@H](C)[C@H](NC(=O)C1CC1(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, 2,2-difluorocyclopropanecarboxylic acid and T₃P instead of TFAA to afford rac-2,2-difluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide (Example 53) as a white solid.

¹H NMR (400 MHz, DMSO-d₆) δ: ppm 11.15 (d, J=12.0 Hz, 1H), 8.74 (dd, J=26.8 Hz, 8.8 Hz, 1H), 7.76 (s, 1H), 7.00-6.80 (m, 1H), 4.30-4.20 (m, 1H), 4.14 (d, J=2.0 Hz, 1H), 3.90-3.75 (m, 3H), 3.45 (dd, J=13.0 Hz, 4.4 Hz, 1H), 3.20-3.10 (m, 2H), 2.70-2.60 (m, 2H), 2.20-2.10 (m, 1H), 1.95-1.70 (m, 4H), 1.66-1.58 (m, 1H), 1.55-1.44 (m, 2H), 1.15-1.07 (m, 1H), 1.04 (d, J=4.8 Hz, 3H), 0.91 (s, 2H), 0.89-0.79 (m, 7H).

MS obsd. (ESI⁺) [(M+H)⁺]: 578.3.

### Example 54

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide

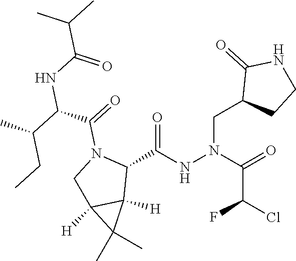 [SMILES: CC[C@H](C)[C@H](NC(=O)C(C)C)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid and isobutyryl chloride instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide (Example 54) as a white solid.

¹H NMR (400 MHz, DMSO-d₆) δ: ppm 11.13 (s, 1H), 8.12 (d, J=8.4 Hz, 1H), 7.76 (s, 1H), 6.90 (d, J=50.4 Hz, 1H), 4.23-4.16 (m, 1H), 4.12 (s, 1H), 3.98-3.92 (m, 1H), 3.89-3.74 (m, 2H), 3.45 (dd, J=14.0 Hz, 4.4 Hz, 1H), 3.21-3.11 (m, 2H), 2.64-2.55 (m, 1H), 2.46-2.39 (m, 1H), 2.20-2.11 (m, 1H), 1.83-1.70 (m, 2H), 1.64-1.56 (m, 1H), 1.52-1.48 (m, 1H), 1.49-1.41 (m, 1H), 1.29-1.24 (m, 1H), 1.05 (s, 3H), 0.97 (d, J=6.8 Hz, 3H), 0.94-0.86 (m, 6H), 0.83-0.76 (m, 6H).

MS obsd. (ESI⁺) [(M+H)⁺]: 544.4.

### Example 55

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-propanamide

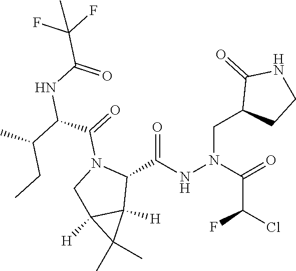 [SMILES: CC[C@H](C)[C@H](NC(=O)C(C)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, 2,2-difluoropropionic acid and T₃P instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-propanamide (Example 55) as a white solid.

¹H NMR (400 MHz, DMSO-d₆) δ: ppm 11.15 (s, 1H), 9.04 (d, J=8.0 Hz, 1H), 7.76 (s, 1H), 6.87 (d, J=50.4 Hz, 1H), 4.25-4.10 (m, 2H), 3.94-3.85 (m, 2H), 3.83-3.76 (m, 1H), 3.45 (dd, J=14.0, 4.4 Hz, 1H), 3.23-3.10 (m, 2H), 2.64-2.55 (m, 1H), 2.30-2.10 (m, 1H), 2.00-1.87 (m, 1H), 1.84-1.77 (m, 1H), 1.77-1.67 (m, 3H), 1.66-1.61 (m, 1H), 1.56-1.52 (m, 1H), 1.50-1.42 (m, 1H), 1.29-1.19 (m, 1H), 1.16-1.06 (m, 1H), 1.05 (s, 3H), 0.91 (s, 3H), 0.85-0.79 (m, 5H).

MS obsd. (ESI⁺) [(M+H)⁺]: 566.3.

### Example 56

### Rac-2-fluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide

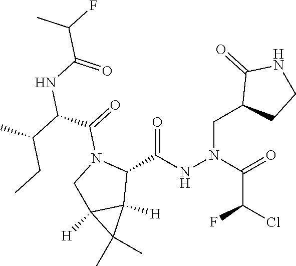 [SMILES: CC[C@H](C)[C@H](NC(=O)C(C)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, 2-fluoropropionic acid and T₃P instead of TFAA to afford rac-2-fluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide (Example 56) as a white solid.

¹H NMR (400 MHz, DMSO-d₆) δ: ppm 11.14 (d, J=4.0 Hz, 1H), 8.45 (dd, J=15.6, 8.0 Hz, 1H), 7.76 (s, 1H), 6.85 (dd, J=50.4, 4.8 Hz, 1H), 5.10-4.90 (m, 1H), 4.27-4.11 (m, 2H), 3.95-3.75 (m, 3H), 3.45 (dd, J=14.0, 4.4 Hz, 1H), 3.22-3.11 (m, 2H), 2.64-2.56 (m, 1H), 2.25-2.10 (m, 1H), 1.90-1.70 (m, 2H), 1.66-1.57 (m, 1H), 1.53 (d, J=7.6 Hz, 1H), 1.49-1.42 (m, 1H), 1.41-1.31 (m, 3H), 1.13-1.07 (m, 1H), 1.05 (s, 3H), 0.91 (d, J=4.4 Hz, 3H), 0.86-0.77 (m, 6H).

MS obsd. (ESI⁺) [(M+H)⁺]: 548.3.

### Example 57

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide

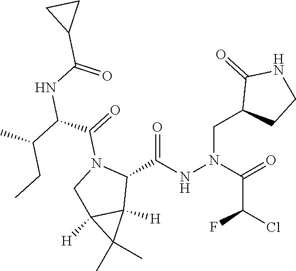 [SMILES: CC[C@H](C)[C@H](NC(=O)C1CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid and cyclopropanecarbonyl chloride instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide (Example 57) as a white solid.

¹H NMR (400 MHz, DMSO-d₆) δ: ppm 11.12 (s, 1H), 8.44 (d, J=8.4 Hz, 1H), 7.76 (s, 1H), 6.90 (d, J=50.4 Hz, 1H), 4.23-4.15 (m, 1H), 4.12 (s, 1H), 3.95-3.88 (m, 1H), 3.86-3.75 (m, 2H), 3.44 (dd, J=14.0, 4.4 Hz, 1H), 3.20-3.10 (m, 2H), 2.65-2.55 (m, 1H), 2.21-2.10 (m, 1H), 1.85-1.70 (m, 2H), 1.68-1.58 (m, 2H), 1.55-1.45 (m, 2H), 1.15-1.06 (m, 1H), 1.04 (s, 3H), 0.88 (s, 3H), 0.84-0.78 (m, 6H), 0.70-0.60 (m, 3H), 0.58-0.51 (m, 1H).

MS obsd. (ESI⁺) [(M+H)⁺]: 542.3.

### Example 58

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide

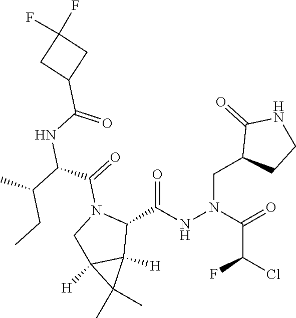 [SMILES: CC[C@H](C)[C@H](NC(=O)C1CC(F)(F)C1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, 3,3-difluorocyclobutanecarboxylic acid and T₃P instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide (Example 58) as a white solid.

¹H NMR (400 MHz, DMSO-d₆) δ: ppm 11.13 (s, 1H), 8.47 (d, J=8.0 Hz, 1H), 7.76 (s, 1H), 6.90 (d, J=50.4 Hz, 1H), 4.25-4.16 (m, 1H), 4.12 (s, 1H), 3.95-3.75 (m, 3H), 3.45 (dd, J=14.0, 4.0 Hz, 1H), 3.21-3.11 (m, 2H), 2.99-2.87 (m, 1H), 2.72-2.62 (m, 4H), 2.60-2.57 (m, 1H), 2.21-2.10 (m, 1H), 1.85-1.68 (m, 2H), 1.66-1.56 (m, 1H), 1.52 (d, J=7.6 Hz, 1H), 1.49-1.40 (m, 1H), 1.13-1.06 (m, 1H), 1.05 (s, 3H), 0.90 (s, 3H), 0.85-0.76 (m, 6H).

MS obsd. (ESI⁺) [(M+H)⁺]: 592.3.

### Example 59

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide

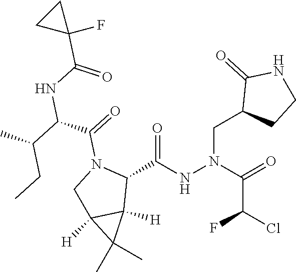 [SMILES: CC[C@H](C)[C@H](NC(=O)C1(F)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, 1-fluorocyclopropane-1-carboxylic acid and T₃P instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide (Example 59) as a white solid.

¹H NMR (400 MHz, DMSO-d₆) δ: ppm 11.13 (s, 1H), 8.54 (d, J=8.0 Hz, 1H), 7.80 (s, 1H), 6.97 (d, J=50.4 Hz, 1H), 4.57-4.48 (m, 1H), 3.98 (d, J=4.0 Hz, 1H), 3.97-3.83 (m, 1H), 3.78-3.62 (m, 2H), 3.39 (dd, J=12.0 Hz, 4.0 Hz, 1H), 3.21-3.10 (m, 2H), 2.77-2.71 (m, 1H), 2.61-2.57 (m, 1H), 2.19-2.11 (m, 1H), 1.94-1.55 (m, 10H), 1.42-1.30 (m, 1H), 0.96 (s, 9H).

MS obsd. (ESI⁺) [(M+H)⁺]: 560.3

### Example 60

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-methyl-cyclopropanecarboxamide

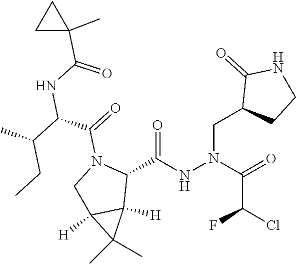 [SMILES: CC[C@H](C)[C@H](NC(=O)C1(C)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, 1-methylcyclopropane-1-carboxylic acid and T₃P instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-methyl-cyclopropanecarboxamide (Example 60) as a white solid.

¹H NMR (400 MHz, DMSO-d₆) δ: ppm 11.10 (s, 1H), 7.75 (s, 1H), 7.59 (d, J=8.0 Hz, 1H), 6.87 (d, J=50.4 Hz, 1H), 4.28-4.16 (m, 2H), 4.11 (s, 1H), 4.05-3.96 (m, 1H), 3.92-3.68 (m, 1H), 3.43 (m, 1H), 3.21-3.11 (m, 2H), 2.61-2.55 (m, 2H), 2.20-2.05 (m, 1H), 1.98-1.84 (m, 1H), 1.82-1.70 (m, 1H), 1.62-1.55 (m, 1H), 1.54-1.40 (m, 2H), 1.23 (s, 3H), 1.10-1.05 (m, 1H), 1.03 (s, 3H), 1.00-0.95 (m, 1H), 0.88 (s, 3H), 0.85-0.75 (m, 6H), 0.61-0.39 (m, 2H).

MS obsd. (ESI⁺) [(M+H)⁺]: 556.3.

### Example 61

### 2,2-dichloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]acetamide

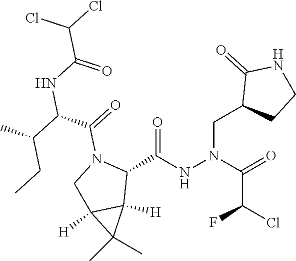 [SMILES: CC[C@H](C)[C@H](NC(=O)C(Cl)Cl)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid, dichloroacetic acid and T₃P instead of TFAA to afford 2,2-dichloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]acetamide (Example 61) as a white solid.

¹H NMR (400 MHz, DMSO-d₆) δ: ppm 11.17 (s, 1H), 9.00 (d, J=8.0 Hz, 1H), 7.76 (s, 1H), 6.85 (d, J=50.4 Hz, 1H), 6.43 (s, 1H), 4.26-4.19 (m, 1H), 4.15 (s, 1H), 3.95-3.87 (m, 1H), 3.86-3.75 (m, 2H), 3.46 (dd, J=13.6 Hz, 4.4 Hz, 1H), 3.23-3.05 (m, 2H), 2.64-2.56 (m, 1H), 2.30-2.10 (m, 1H), 1.85-1.70 (m, 2H), 1.68-1.59 (m, 1H), 1.55 (d, J=7.2 Hz, 1H), 1.51-1.41 (m, 1H), 1.15-1.08 (m, 1H), 1.06 (s, 3H), 0.94 (s, 3H), 0.87-0.79 (m, 6H).

MS obsd. (ESI⁺) [(M+H)⁺]: 584.0.

### Example 62

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,3,3,3-pentafluoro-propanamide

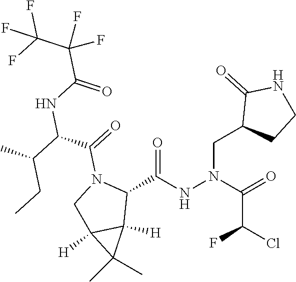 [SMILES: CC[C@H](C)[C@H](NC(=O)C(F)(F)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid and 2,2,3,3,3-pentafluoropropanoyl 2,2,3,3,3-pentafluoropropanoate instead of TFAA to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,3,3,3-pentafluoro-propanamide (Example 62) as a white solid.

¹H NMR (400 MHz, DMSO-d₆) δ: ppm 11.18 (s, 1H), 10.06 (d, J=7.6 Hz, 1H), 7.76 (s, 1H), 6.83 (d, J=50.4 Hz, 1H), 4.31-4.21 (m, 1H), 3.97-3.86 (m, 2H), 3.85-3.75 (m, 1H), 3.45 (dd, J=4.4, 14.0 Hz, 1H), 3.22-3.09 (m, 2H), 2.63-2.55 (m, 2H), 2.21-2.10 (m, 1H), 2.05-1.95 (m, 1H), 1.83-1.72 (m, 1H), 1.55 (d, J=7.6 Hz, 1H), 1.59-1.51 (m, 1H), 1.48-1.39 (m, 1H), 1.16-1.08 (m, 1H), 1.04 (s, 3H), 0.95-0.74 (m, 9H).

MS obsd. (ESI⁺) [(M+H)⁺]: 620.3.

### Example 63

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide

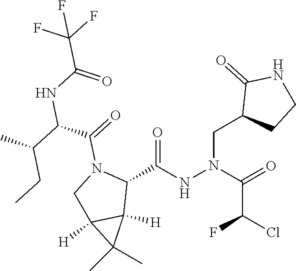 [SMILES: CC[C@H](C)[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 3, by using (2S,3S)-2-(benzyloxycarbonylamino)-3-methyl-pentanoic acid instead of (2S)-2-(benzyloxycarbonylamino)-4-methyl-pentanoic acid to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide (Example 63) as a white solid.

¹H NMR (400 MHz, DMSO-d₆) δ: ppm 11.16 (s, 1H), 9.97-9.95 (m, 1H), 7.76 (s, 1H), 6.91-6.78 (d, J=50.4 Hz, 1H), 4.22-4.16 (m, 2H), 3.92-3.76 (m, 3H), 3.47 (dd, J=4.0, 14.0 Hz, 1H), 3.18-3.13 (m, 2H), 2.63-2.57 (m, 1H), 2.19-2.11 (m, 1H), 1.96-1.87 (m, 1H), 1.81-1.73 (m, 1H), 1.67-1.63 (m, 1H), 1.55 (d, J=7.6 Hz, 1H), 1.49-1.43 (m, 1H), 1.14-1.05 (s, 4H), 0.91-0.79 (m, 9H).

MS obsd. (ESI⁺) [(M+H)+]: 570.2.

### Example 64

### N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide

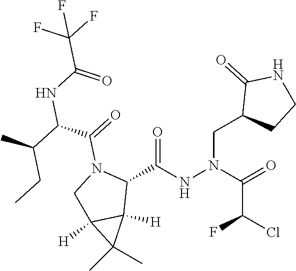 [SMILES: CC[C@@H](C)[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

### Step 1: Preparation of methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate

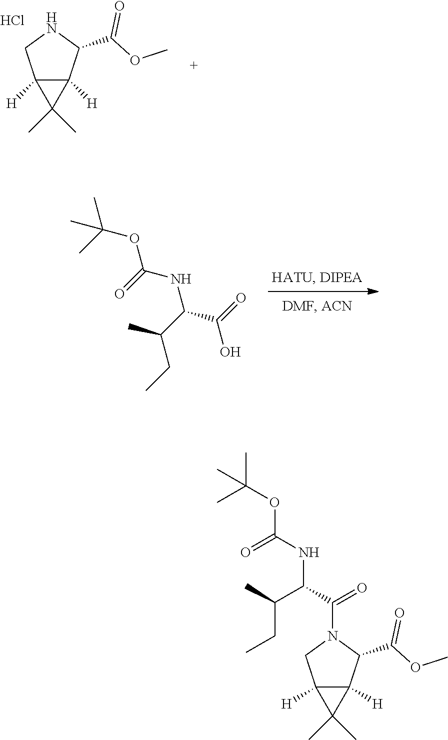 [SMILES: Cl ; COC(=O)[C@H]1NC[C@H]2[C@@H]1C2(C)C ; CC[C@@H](C)[C@H](NC(=O)OC(C)(C)C)C(=O)O ; "HATU, DIPEA" ; CC[C@@H](C)[C@H](NC(=O)OC(C)(C)C)C(=O)N1C[C@H]2[C@@H]([C@H]1C(=O)OC)C2(C)C]

To a solution of Boc-L-isoleucine (500 mg, 2.16 mmol) (GL Biochem, CAS number: 35264-07-4) in DMF (5 mL) and ACN (20 mL) was added DIPEA (1400 mg, 10.81 mmol) and HATU (904 mg, 2.38 mmol) at 0° C. After the addition of methyl (1R,2S,5S)-6,6-dimethyl-3-azabicyclo[3.1.0]hexane-2-carboxylate; hydrochloride was added into above solution, the mixture was stirred 25° C. for 1 h. The mixture was diluted with EtOAc (100 mL) and washed with brine (60 mL×3). The organic phase was dried over Na₂SO₄, filtered and concentrated in vacuum and the residue was purified by silica gel column eluted with PE to PE/EtOAc=1/1 to afford methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate (760 mg) as a yellow foam.

MS obsd. (ESI⁺) [(M+H)⁺]: 383.2.

### Step 2: Preparation of (1R,2S,5S)-3-[(2S,3R)-2-(tert-butoxycarbonylamino)-3-methyl-pentanoyl]-6,6-dimethyl-3-azabicyclo[3.1.0]hexane-2-carboxylic acid

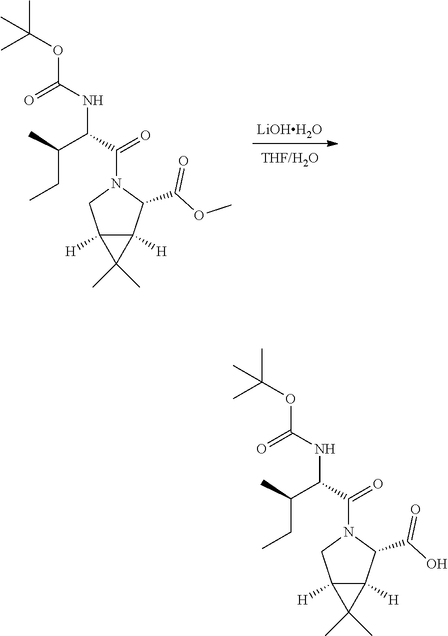 [SMILES: CC[C@@H](C)[C@H](NC(=O)OC(C)(C)C)C(=O)N1C[C@H]2[C@@H]([C@H]1C(=O)OC)C2(C)C ; "LiOH·H2O" ; CC[C@@H](C)[C@H](NC(=O)OC(C)(C)C)C(=O)N1C[C@H]2[C@@H]([C@H]1C(=O)O)C2(C)C]

To a solution of methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate (760 mg, 1.99 mmol) in water (5 mL) and THF (20 mL) was added LiOH·H₂O (250 mg, 5.96 mmol). The mixture was stirred at 25° C. for 1 h. The mixture was acidified with 1 N HCl to pH=4 and diluted with H₂O (30 mL). The mixture was extracted with EtOAc (50 mL×2). The organic phase was washed with brine (60 mL), dried over Na₂SO₄, filtered, and concentrated in vacuum to afford (1R,2S,5S)-3-[(2S,3R)-2-(tert-butoxycarbonylamino)-3-methyl-pentanoyl]-6,6-dimethyl-3-azabicyclo[3.1.0]hexane-2-carboxylic acid (600 mg) as a yellow foam.

MS obsd. (ESI⁺) [(M+H)⁺]: 369.2.

### Step 3: Preparation of (1R,2S,5S)-3-[(2S,3R)-2-amino-3-methyl-pentanoyl]-6,6-dimethyl-3-azabicyclo[3.1.0]hexane-2-carboxylic acid; hydrochloride

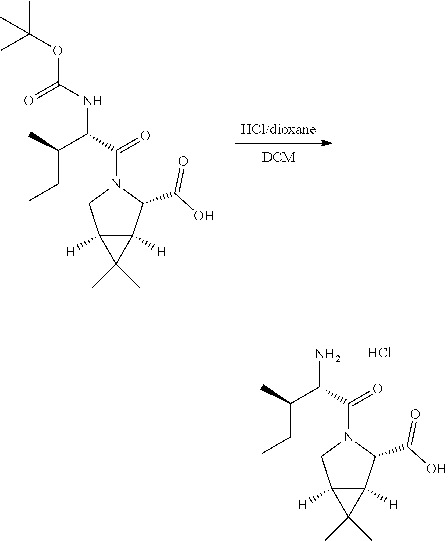 [SMILES: CC[C@@H](C)[C@H](NC(=O)OC(C)(C)C)C(=O)N1C[C@H]2[C@@H]([C@H]1C(=O)O)C2(C)C ; "HCl/dioxane" ; Cl ; CC[C@@H](C)[C@H](N)C(=O)N1C[C@H]2[C@@H]([C@H]1C(=O)O)C2(C)C]

To a solution of (1R,2S,5S)-3-[(2S,3R)-2-(tert-butoxycarbonylamino)-3-methyl-pentanoyl]-6,6-dimethyl-3-azabicyclo[3.1.0]hexane-2-carboxylic acid (600 mg, 1.63 mmol) in DCM (20 mL) was added HCl/dioxane (20 mL, 4 N). The mixture was stirred 25° C. for 1 h. The mixture was concentrated in vacuum to afford (JR,2S,5S)-3-[(2S,3R)-2-amino-3-methyl-pentanoyl]-6,6-dimethyl-3-azabicyclo[3.1.0]hexane-2-carboxylic acid; hydrochloride (495 mg) as a yellow foam.

MS obsd. (ESI⁺) [(M+H)⁺]: 269.1.

### Step 4: Preparation of (1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carboxylic acid

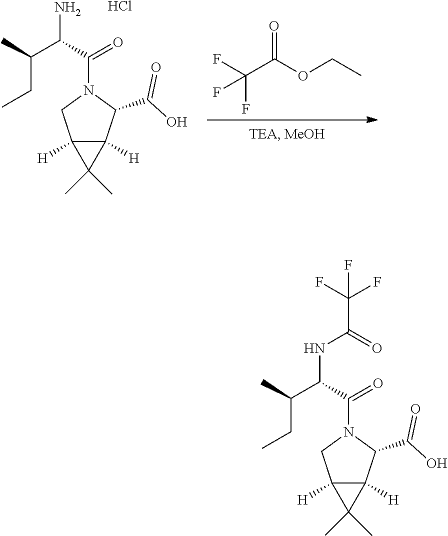

To a solution of (1R,2S,5S)-3-[(2S,3R)-2-amino-3-methyl-pentanoyl]-6,6-dimethyl-3-azabicyclo[3.1.0]hexane-2-carboxylic acid; hydrochloride (490 mg, 1.61 mmol) in methanol (5 mL) was added Et₃N (1.6 g, 16.08 mmol) and ethyl trifluoroacetate (685 mg, 4.82 mmol). The mixture was stirred at 50° C. for 12 h. The mixture was concentrated in vacuum and the residue was dissolved in EtOAc (150 mL) and washed with 1 N HCl (40 mL), brine (50 mL) and dried over Na₂SO₄. After filtration and concentration, (1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carboxylic acid (500 mg) was obtained as yellow oil.

MS obsd. (ESI⁺) [(M+H)⁺]: 365.1.

### Step 5: Preparation of tert-butyl N-[[(1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate

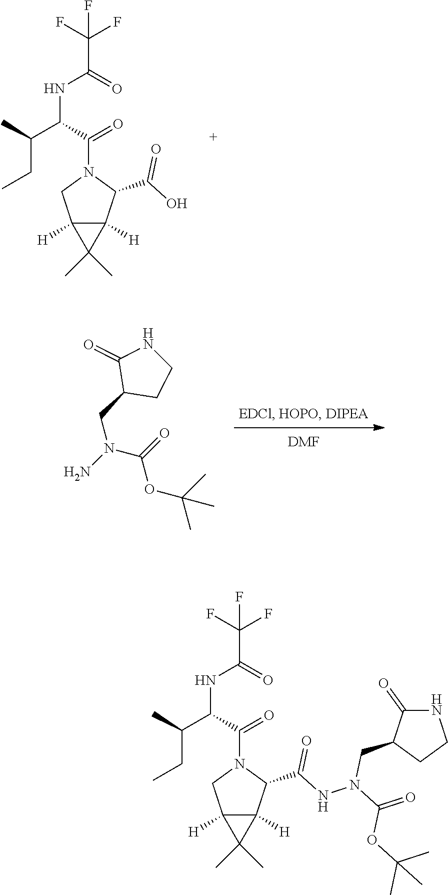

To a solution of (1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carboxylic acid (500 mg, 1.37 mmol) in DMF (8 mL) was added DIPEA (709 mg, 5.49 mmol), EDCI (316 mg, 1.65 mmol), HOPO (183 mg, 1.65 mmol) and tert-butyl N-amino-N-[[(3S)-2-oxopyrrolidin-3-yl]methyl]carbamate (378 mg, 1.65 mmol, Intermediate 9). The mixture was stirred at 25° C. for 12 h. The mixture was diluted with EtOAc (100 mL), washed with brine (50 mL×3), 1 N HCl (30 mL) and dried over Na₂SO₄. After filtration and concentration, tert-butyl N-[[(1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate (600 mg) was obtained as yellow oil.

MS obsd. (ESI⁺) [(M+H)⁺]: 576.4.

### Step 6: Preparation of N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-2,2,2-trifluoro-acetamide

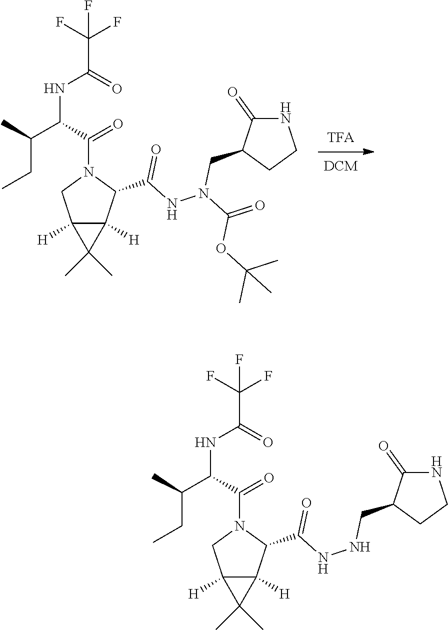 [SMILES: CC[C@@H](C)[C@H](NC(=O)C(F)(F)F)C(=O)N1C[C@H]2[C@@H]([C@H]1C(=O)NN(C[C@@H]1CCNC1=O)C(=O)OC(C)(C)C)C2(C)C ; CC[C@@H](C)[C@H](NC(=O)C(F)(F)F)C(=O)N1C[C@H]2[C@@H]([C@H]1C(=O)NNC[C@@H]1CCNC1=O)C2(C)C ; "TFA"]

To a solution of tert-butyl N-[[(1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate (600 mg, 1.04 mmol) in DCM (10 mL) was added TFA (10.0 mL). The mixture was stirred at 25° C. for 1 h. The mixture was concentrated and the residue was purified by reverse flash eluted with ACN in H₂O (0.1% HCl)=0˜55% to afford N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-2,2,2-trifluoro-acetamide; hydrochloride (511 mg) as a yellow foam.

MS obsd. (ESI⁺) [(M+H)⁺]: 476.1.

### Step 7: Preparation of N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide (Example 64)

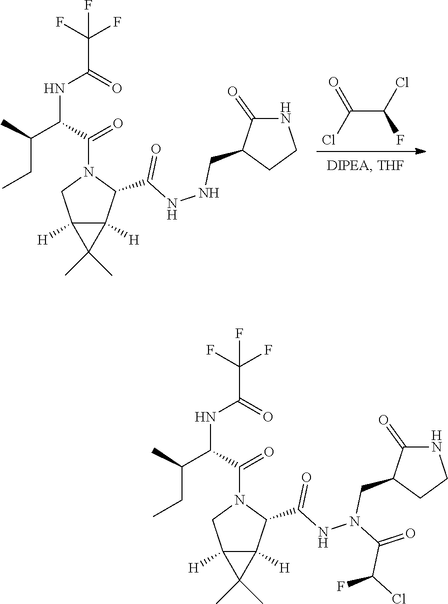

To a solution of N-[(1S,2R)-1-[(1R,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-2,2,2-trifluoro-acetamide hydrochloride (500 mg, 0.97 mmol) in THF (150 mL) was added DIPEA (2524 mg, 19.53 mmol) in one portion. The mixture was cooled to 0° C. and a solution of (2R)-2-chloro-2-fluoro-acetyl chloride (639 mg, 1.95 mmol, Intermediate 10) in THF (10 mL) was added dropwise over 5 min. The resulting mixture was stirred at 0° C. for 30 min. The reaction was quenched with MeOH (2 mL), followed by 4 N HCl/dioxane (4 mL) at 0° C. Then the mixture was concentrated and the residue was purified by reverse flash eluted with ACN in H₂O (0.1% FA)=0˜50% to afford N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide (150 mg, Example 64) as a white solid.

¹H NMR (400 MHz, DMSO-d₆) δ: ppm 11.18 (s, 1H), 9.88 (d, J=8.0 Hz, 1H), 7.77 (s, 1H), 6.82 (d, J=50.4 Hz, 1H), 4.30-4.22 (m, 1H), 4.17 (s, 1H), 3.93-3.76 (m, 3H), 3.45 (dd, J=13.6 Hz, 4.0 Hz, 1H), 3.22-3.11 (m, 2H), 2.64-2.56 (m, 1H), 2.20-2.10 (m, 1H), 1.98-1.86 (m, 1H), 1.84-1.70 (m, 1H), 1.69-1.59 (m, 1H), 1.57-1.52 (m, 1H), 1.50-1.40 (m, 1H), 1.09-1.01 (m, 4H), 0.91 (s, 3H), 0.86 (d, J=6.8 Hz, 3H), 0.84-0.78 (m, 3H).

MS obsd. (ESI⁺) [(M+H)⁺]: 570.3.

### Example 65

### N-[(1S)-1-(1-bicyclo[1.1.1]pentanylmethyl)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide

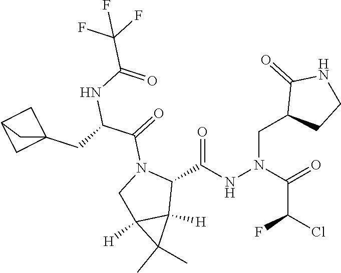 [SMILES: CC1(C)[C@@H]2[C@@H](C(=O)NN(C[C@@H]3CCNC3=O)C(=O)[C@H](F)Cl)N(C(=O)[C@H](CC34CC(C3)C4)NC(=O)C(F)(F)F)C[C@@H]21]

The title compound was prepared in analogy to the procedure described for the preparation of Example 64, by using (2S)-3-(1-bicyclo[1.1.1]pentanyl)-2-(tert-butoxycarbonylamino)propanoic acid (Intermediate 19) instead of Boc-L-isoleucine to afford N-[(1S)-1-(1-bicyclo[1.1.1]pentanylmethyl)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide (Example 65) as a white solid.

¹H NMR (400 MHz, DMSO-d₆) δ: ppm 11.12 (s, 1H), 10.00-9.90 (m, 1H), 7.76 (s, 1H), 6.75 (d, J=50.4 Hz, 1H), 4.43-4.48 (m, 1H), 4.15 (s, 1H), 3.83-3.76 (m, 2H), 3.71-3.66 (m, 1H), 3.48-3.38 (m, 2H), 3.20-3.14 (m, 2H), 2.62-2.59 (m, 1H), 2.17-2.11 (m, 1H), 1.85-1.79 (m, 3H), 1.71-1.65 (m, 4H), 1.62-1.55 (m, 4H), 1.06 (s, 3H), 0.92 (s, 3H).

MS obsd. (ESI⁺) [(M+H)⁺]: 594.1.

### Example 66

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-2,2,2-trifluoro-acetamide

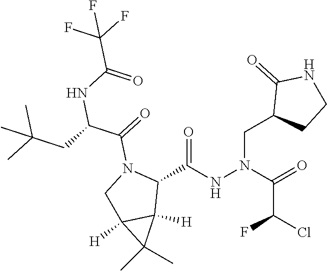 [SMILES: CC(C)(C)C[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 20, by using (2S)-2-(benzyloxycarbonylamino)-4,4-dimethyl-pentanoic acid (Intermediate 20) instead of Cbz-Phe-OH to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-2,2,2-trifluoro-acetamide (Example 66) as a white solid.

¹H NMR (400 MHz, CDCl₃) δ: ppm 10.83-10.23 (m, 1H), 7.34-7.27 (m, 1H), 6.60 (d, J=50.4 Hz, 1H), 6.35-6.22 (m, 1H), 4.82-4.77 (m, 1H), 4.58-4.37 (m, 2H), 4.01-3.80 (m, 2H), 3.44-3.31 (m, 2H), 2.95-2.79 (m, 2H), 2.45-2.25 (m, 1H), 1.81-1.72 (m, 2H), 1.66-1.62 (m, 1H), 1.45-1.39 (m, 2H), 1.13-1.07 (m, 3H), 0.97-0.94 (m, 12H).

MS obsd. (ESI⁺) [(M+H)⁺]: 584.2.

### Example 67

### N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-(4-pyridylmethyl)ethyl]-2,2,2-trifluoro-acetamide

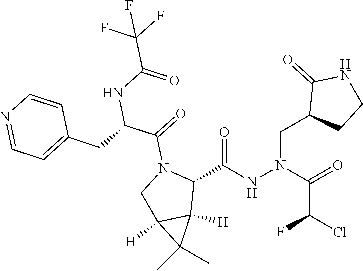 [SMILES: CC1(C)[C@@H]2[C@@H](C(=O)NN(C[C@@H]3CCNC3=O)C(=O)[C@H](F)Cl)N(C(=O)[C@H](Cc3ccncc3)NC(=O)C(F)(F)F)C[C@@H]21]

The title compound was prepared in analogy to the procedure described for the preparation of Example 64, by using Boc-L-4-pyridylalanine (Bide, CAS number: 37535-57-2) instead of Boc-L-isoleucine to afford N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-(4-pyridylmethyl)ethyl]-2,2,2-trifluoro-acetamide (Example 67) as a white solid.

¹H NMR (400 MHz, DMSO) δ: ppm 11.11 (s, 1H), 10.08 (d, J=8.0 Hz, 1H), 8.45 (d, J=5.6 Hz, 2H), 7.76 (s, 1H), 7.32 (d, J=6.0 Hz, 2H), 6.78 (d, J=50.4 Hz, 1H), 4.73-4.71 (m, 1H), 4.16 (s, 1H), 3.86-3.80 (m, 1H), 3.71 (d, J=10.8 Hz, 1H), 3.47-3.42 (m, 1H), 3.23-3.13 (m, 2H), 3.04-2.95 (m, 2H), 2.67-2.52 (m, 1H), 2.32-2.16 (m, 1H), 1.80-1.75 (m, 1H), 1.65-1.63 (m, 1H), 1.61-1.59 (m, 1H), 1.05 (s, 3H), 0.91 (s, 3H).

MS obsd. (ESI⁺) [(M+H)⁺]: 605.1.

### Example 68

### N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-(1-methylcyclopropyl)-2-oxo-ethyl]-2,2,2-trifluoro-acetamide

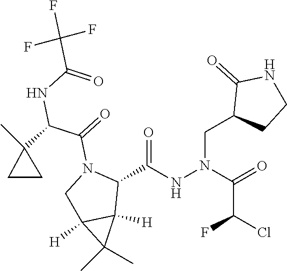 [SMILES: CC1([C@H](NC(=O)C(F)(F)F)C(=O)N2C[C@H]3[C@@H]([C@H]2C(=O)NN(C[C@@H]2CCNC2=O)C(=O)[C@H](F)Cl)C3(C)C)CC1]

The title compound was prepared in analogy to the procedure described for the preparation of Example 64, by using (2S)-2-(tert-butoxycarbonylamino)-2-(1-methylcyclopropyl)acetic acid (Intermediate 16) instead of Boc-L-isoleucine to afford N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-(1-methylcyclopropyl)-2-oxo-ethyl]-2,2,2-trifluoro-acetamide (Example 68) as a white solid.

¹H NMR (400 MHz, DMSO-d₆) δ: ppm 11.16 (s, 1H), 9.80 (d, J=6.4 Hz, 1H), 7.75 (s, 1H), 6.84-6.63 (m, 1H), 4.38 (d, J=6.0 Hz, 1H), 4.13 (s, 1H), 3.91-3.66 (m, 3H), 3.44 (dd, J=4.4, 13.6 Hz, 1H), 3.21-3.06 (m, 2H), 2.63-2.58 (m, 1H), 2.23-2.09 (m, 1H), 1.79-1.73 (m, 1H), 1.71-1.64 (m, 1H), 1.53 (d, J=7.6 Hz, 1H), 1.06 (d, J=2.8 Hz, 6H), 0.96 (s, 3H), 0.77-0.65 (m, 2H), 0.37-0.25 (m, 2H).

MS obsd. (ESI⁺) [(M+H)⁺]: 568.3.

### Example 69

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methylsulfanyl-propyl]-2,2,2-trifluoro-acetamide

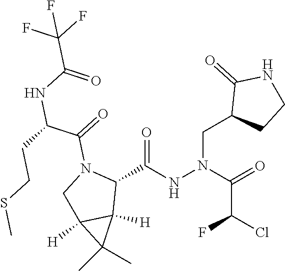 [SMILES: CSCC[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 20, by using Cbz-Met-OH (Energy chemical, CAS number: 1152-62-1) instead of Cbz-Phe-OH to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methylsulfanyl-propyl]-2,2,2-trifluoro-acetamide (Example 69) as a white solid.

¹H NMR (400 MHz, DMSO-d₆) δ: ppm 11.15 (s, 1H), 9.93 (d, J=6.0 Hz, 1H), 7.76 (s, 1H), 6.74 (d, J=50.4 Hz, 1H), 4.58-4.52 (m, 1H), 4.18 (s, 1H), 3.91-3.85 (m, 1H), 3.82-3.74 (m, 2H), 3.43 (dd, J=14.0 Hz, 4.4 Hz, 1H), 3.18-3.12 (m, 2H), 2.62-2.54 (m, 1H), 2.33-2.23 (m, 2H), 2.20-2.10 (m, 1H), 2.20 (s, 3H), 1.96-1.87 (m, 2H), 1.81-1.71 (m, 1H), 1.68-1.62 (m, 1H), 1.56 (d, J=7.2 Hz, 1H), 1.06 (s, 3H), 0.93 (d, J=10.0 Hz, 3H).

MS obsd. (ESI⁺) [(M+H)⁺]: 588.2.

### Example 70

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-dichloro-acetamide

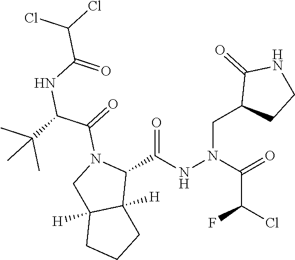 [SMILES: CC(C)(C)[C@H](NC(=O)C(Cl)Cl)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using dichloroacetic acid and T₃P instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-dichloro-acetamide (Example 70) as a white solid.

¹H NMR (400 MHz, DMSO-d₆) δ: ppm 11.13 (s, 1H), 8.69 (d, J=8.4 Hz, 1H), 7.77 (s, 1H), 6.98 (d, J=50.4 Hz, 1H), 6.66 (d, J=2.0 Hz, 1H), 4.48-4.40 (m, 1H), 3.98 (d, J=6.0 Hz, 1H), 3.90 (dd, J=7.2, 10.4 Hz, 1H), 3.79-3.63 (m, 2H), 3.39 (dd, J=4.0, 13.6 Hz, 1H), 3.22-3.10 (m, 2H), 2.81-2.70 (m, 1H), 2.61-2.54 (m, 1H), 2.54-2.51 (m, 1H), 2.21-2.11 (m, 1H), 1.93-1.70 (m, 5H), 1.69-1.59 (m, 1H), 1.43-1.32 (m, 1H), 0.95 (s, 9H).

MS obsd. (ESI⁺) [(M+H)⁺]: 584.1.

### Example 71

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,3,3,3-pentafluoro-propanamide

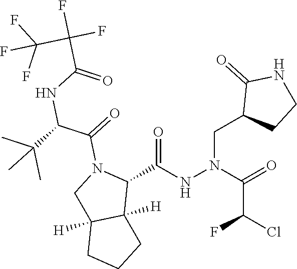 [SMILES: CC(C)(C)[C@H](NC(=O)C(F)(F)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using 2,2,3,3,3-pentafluoropropanoyl 2,2,3,3,3-pentafluoropropanoate instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,3,3,3-pentafluoro-propanamide (Example 71) as a white solid.

¹H NMR (400 MHz, DMSO-d₆) δ: ppm 11.13 (s, 1H), 9.41 (dd, J=26.8 Hz, 8.0 Hz 1H), 7.78 (s, 1H), 6.94 (d, J=50.8 Hz, 1H), 4.57 (d, J=7.2 Hz, 1H), 3.99 (d, J=6.0 Hz, 1H), 3.92-3.72 (m, 2H), 3.66 (dd, J=10.40 Hz, 2.40 Hz, 1H), 3.39 (dd, J=14.4 Hz, 4.0 Hz, 1H), 3.22-3.06 (m, 2H), 2.79-2.69 (m, 1H), 2.61-2.54 (m, 1H), 2.53-2.41 (m, 1H), 2.20-2.10 (m, 1H), 1.93-1.55 (m, 6H), 1.40-1.27 (m, 1H), 0.98 (s, 9H).

MS obsd. (ESI⁺) [(M+H)⁺]: 620.2.

### Example 72

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]-2,2,2-trifluoro-acetamide

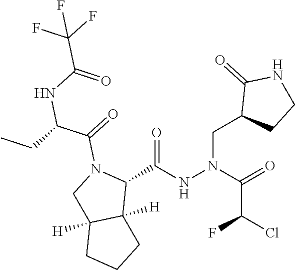 [SMILES: CC[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

### Step 1: Preparation of benzyl (3S,3aS,6aR)-2-[(2S)-2-(benzyloxycarbonylamino)butanoyl]-3,3a,4,5,6,6a-hexahydro-1H-cyclopenta[c]pyrrole-3-carboxylate

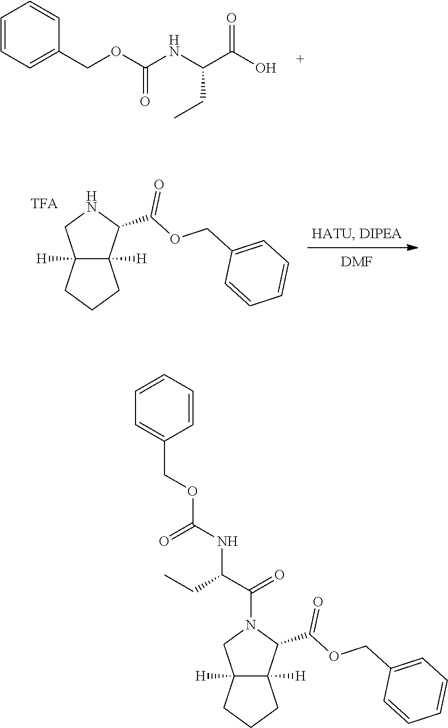

To a solution of (S)-2-(((benzyloxy)carbonyl)amino)butanoic acid (660 mg, 2.78 mmol) in DMF (20 mL) was added DIPEA (1.8 g, 13.91 mmol) and HATU (1.27 g, 3.34 mmol). Then benzyl (3S,3aS,6aR)-1,2,3,3a,4,5,6,6a-octahydrocyclopenta[c]pyrrole-3-carboxylate; 2,2,2-trifluoroacetic acid (1.0 g, 2.78 mmol, Intermediate 21) was added into at 0° C. After the addition, the mixture was stirred at 25° C. for 12 h. The reaction was diluted with water (100 mL) and extracted with EtOAc (50 mL×2). The organic phase was washed with saturated aqueous solution of K₂CO₃ (20 mL), 1 N HCl (30 mL) and brine (60 mL×3). The organic phase was dried over Na₂SO₄, filtered and concentrated and the residue was purified by silica gel column eluted with PE to PE/EtOAc=1/1 to afford benzyl (3S,3aS,6aR)-2-[(2S)-2-(benzyloxycarbonylamino)butanoyl]-3,3a,4,5,6,6a-hexahydro-1H-cyclopenta[c]pyrrole-3-carboxylate (891 mg) as colorless oil.

MS obsd. (ESI⁺) [(M+H)⁺]: 465.2.

### Step 2: Preparation of (3S,3aS,6aR)-2-[(2S)-2-aminobutanoyl]-3,3a,4,5,6,6a-hexahydro-1H-cyclopenta[c]pyrrole-3-carboxylic acid

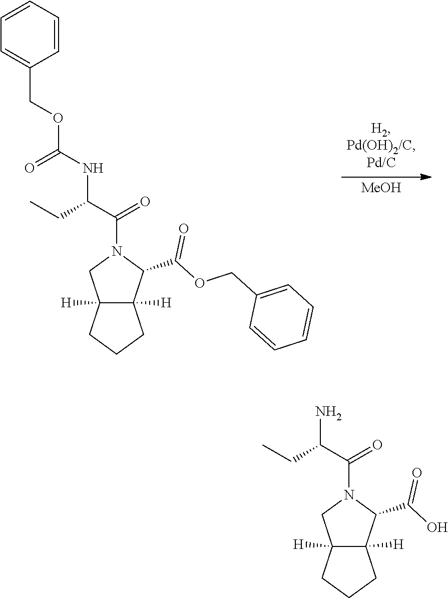 [SMILES: "MeOH" ; CC[C@H](NC(=O)OCc1ccccc1)C(=O)N1C[C@@H]2CCC[C@@H]2[C@H]1C(=O)OCc1ccccc1 ; CC[C@H](N)C(=O)N1C[C@@H]2CCC[C@@H]2[C@H]1C(=O)O]

To a solution of benzyl (3S,3aS,6aR)-2-[(2S)-2-(benzyloxycarbonylamino)butanoyl]-3,3a,4,5,6,6a-hexahydro-1H-cyclopenta[c]pyrrole-3-carboxylate (1.5 g, 3.25 mmol) in THF (15 mL) was added Pd/C (150.0 mg, 10% purity) and Pd(OH)₂ (150.0 mg). The suspension was degassed under vacuum and purged hydrogen for three times. The resulting mixture was stirred at 25° C. for 12 h under a H₂ balloon. The mixture was filtrated through a pad of celite and the filtrate was concentrated in vacuum to afford (3S,3aS,6aR)-2-[(2S)-2-aminobutanoyl]-3,3a,4,5,6,6a-hexahydro-1H-cyclopenta[c]pyrrole-3-carboxylic acid (758 mg) as a white solid.

MS obsd. (ESI⁺) [(M+H)⁺]: 240.9.

### Step 3: Preparation of (3S,3aS,6aR)-2-[(2S)-2-[(2,2,2-trifluoroacetyl)amino]butanoyl]-3,3a,4,5,6,6a-hexahydro-1H-cyclopenta[c]pyrrole-3-carboxylic acid

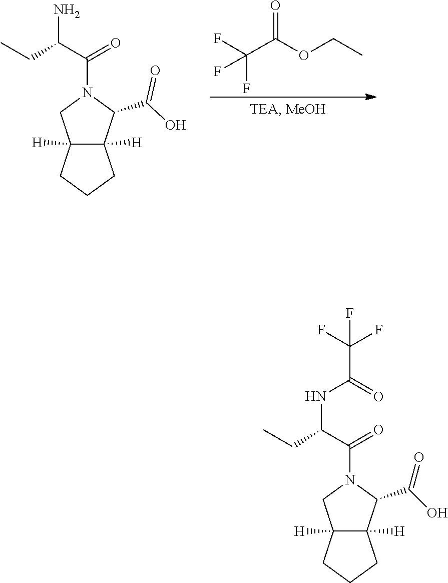

To a solution of (3S,3aS,6aR)-2-[(2S)-2-aminobutanoyl]-3,3a,4,5,6,6a-hexahydro-1H-cyclopenta[c]pyrrole-3-carboxylic acid (758 mg, 3.15 mmol) in methanol (6 mL) was added Et₃N (958 mg, 9.46 mmol). The mixture was cooled to 5° C. and ethyl trifluoroacetate (448 mg, 3.15 mmol) was added. After the addition, the mixture was stirred at 50° C. for 12 h. The reaction was diluted with water (30 mL) and extracted with EtOAc (65 mL×2). The organic phase was washed with 1 N HCl (15 mL), brine (30 mL) and dried over Na₂SO₄. After filtration and concentration, (3S,3aS,6aR)-2-[(2S)-2-[(2,2,2-trifluoroacetyl)amino]butanoyl]-3,3a,4,5,6,6a-hexahydro-1H-cyclopenta[c]pyrrole-3-carboxylic acid (1060 mg) was obtained as yellow oil.

MS obsd. (ESI⁺) [(M+H)⁺]: 337.1.

### Step 4: Preparation of tert-butyl N-[[(3S,3aS,6aR)-2-[(2S)-2-[(2,2,2-trifluoroacetyl)amino]butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate

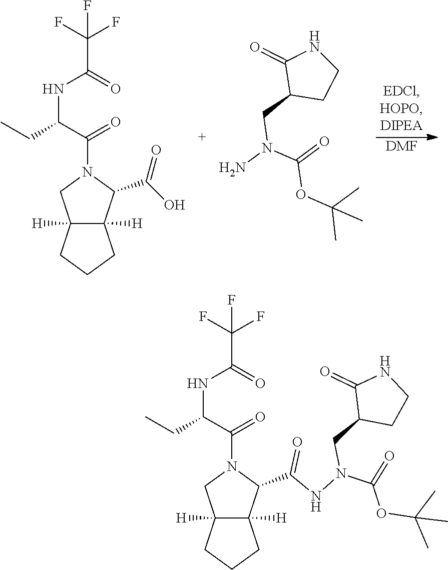

To a solution of (3S,3aS,6aR)-2-[(2S)-2-[(2,2,2-trifluoroacetyl)amino]butanoyl]-3,3a,4,5,6,6a-hexahydro-1H-cyclopenta[c]pyrrole-3-carboxylic acid (1.1 g, 3.22 mmol) in DMF (20 mL) was added DIPEA (1.67 g, 12.9 mmol). Then HOPO (430 mg, 3.87 mmol), EDCI (741 mg, 3.87 mmol) and tert-butyl N-amino-N-[[(3S)-2-oxopyrrolidin-3-yl]methyl]carbamate (739 mg, 3.22 mmol, Intermediate 9) were added at 0° C. The mixture was stirred at 30° C. for 12 h. The reaction mixture was diluted with water (100 mL) and extracted with EtOAc (80 mL×2). The organic phase was washed with 1 N HCl (30 mL), brine (60 mL×3) and dried over Na₂SO₄. After filtration and concentration, tert-butyl N-[[(3S,3aS,6aR)-2-[(2S)-2-[(2,2,2-trifluoroacetyl)amino]butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (1.6 g) was obtained as yellow oil.

MS obsd. (ESI⁺) [(M+H)⁺]: 548.3.

### Step 5: Preparation of N-[(1S)-1-[(3S,3aS,6aR)-3-[[[(3S)-2-oxopyrrolidin-3-yl]methylamino]carbamoyl]-3,3a,4,5,6,6a-hexahydro-1H-cyclopenta[c]pyrrole-2-carbonyl]propyl]-2,2,2-trifluoro-acetamide

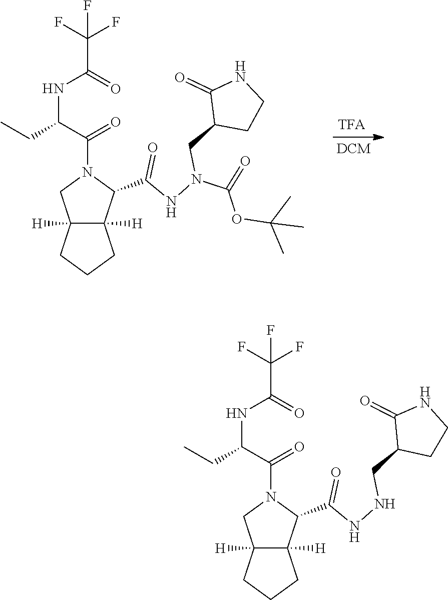

To a solution of tert-butyl N-[[(3S,3aS,6aR)-2-[(2S)-2-[(2,2,2-trifluoroacetyl)amino]butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (1.61 g, 2.93 mmol) in DCM (6 mL) was added TFA (12.0 mL). The mixture was stirred at 25° C. for 1 h. The reaction mixture was concentrated and the residue was purified by reverse flash eluted with ACN in H₂O (0.1% HCl)=0˜60% to afford N-[(1S)-1-[(3S,3aS,6aR)-3-[[[(3S)-2-oxopyrrolidin-3-yl]methylamino]carbamoyl]-3,3a,4,5,6,6a-hexahydro-1H-cyclopenta[c]pyrrole-2-carbonyl]propyl]-2,2,2-trifluoro-acetamide (1.2 g, 2.68 mmol) as a yellow solid.

MS obsd. (ESI⁺) [(M+H)⁺]: 448.0.

### Step 6: Preparation of N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]-2,2,2-trifluoro-acetamide (Example 72)

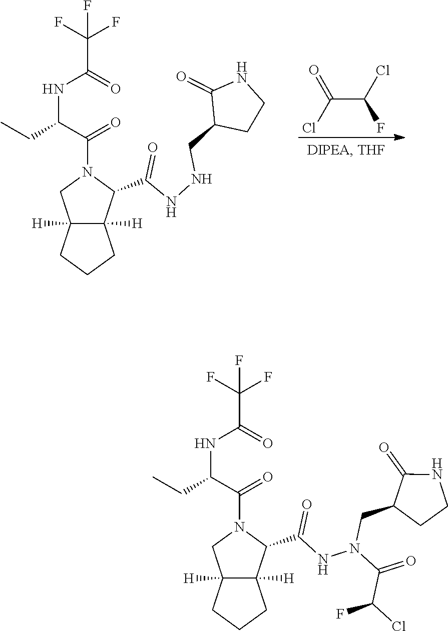 [SMILES: CC[C@H](NC(=O)C(F)(F)F)C(=O)N1C[C@@H]2CCC[C@@H]2[C@H]1C(=O)NNC[C@@H]1CCNC1=O ; O=C(Cl)[C@H](F)Cl ; CC[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

To a solution of N-[(1S)-1-[(3S,3aS,6aR)-3-[[[(3S)-2-oxopyrrolidin-3-yl]methylamino]carbamoyl]-3,3a,4,5,6,6a-hexahydro-1H-cyclopenta[c]pyrrole-2-carbonyl]propyl]-2,2,2-trifluoro-acetamide (1.2 g, 2.68 mmol) in THF (350 mL) was added DIPEA (5.2 g, 40.23 mmol). Then (2R)-2-chloro-2-fluoro-acetyl chloride (702 mg, 5.36 mmol, Intermediate 10) in THF (20 mL) was added at −5° C. dropwise. The mixture was stirred at −5° C. for 1 h. The reaction was quenched with MeOH (10 mL) and acidified by 4 N HCl/dioxane to pH=5 at −10° C. Then the mixture was concentrated in vacuum at 30° C. The residue was purified by reverse flash eluted with ACN in H₂O (0.1% FA)=0˜60% to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]-2,2,2-trifluoro-acetamide (375 mg, Example 72) as a white solid.

¹H NMR (400 MHz, DMSO-d₆) δ: ppm 11.06-10.44 (m, 1H), 9.82 (d, J=6.6 Hz, 1H), 7.79 (s, 1H), 6.97-6.76 (m, 1H), 4.52-4.39 (m, 1H), 3.98-4.14 (m, 1H), 3.96-3.60 (m, 3H), 3.42-3.35 (m, 1H), 3.22-3.10 (m, 2H), 2.83-2.76 (m, 1H), 2.61-2.57 (m, 1H), 2.23-2.07 (m, 1H), 1.92-1.56 (m, 9H), 1.46-1.36 (m, 1H), 0.89 (t, J=7.3 Hz, 3H).

MS obsd. (ESI⁺) [(M+H)⁺]: 542.0.

### Example 73

### N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide

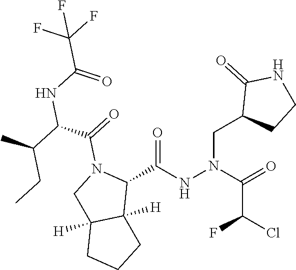 [SMILES: CC[C@@H](C)[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (3S,3aS,6aR)-2-[(2S,3R)-2-amino-3-methyl-pentanoyl]-3,3a,4,5,6,6a-hexahydro-1H-cyclopenta[c]pyrrole-3-carboxylate; 2,2,2-trifluoroacetic acid (Intermediate 22) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide (Example 73) as a white solid.

¹H NMR (400 MHz, DMSO-d₆) δ: ppm 11.10 (s, 1H), 9.87-9.62 (m, 1H), 7.78 (s, 1H), 6.96 (d, J=50.8 Hz, 1H), 4.46-4.29 (m, 1H), 4.14-3.97 (m, 1H), 3.89-3.67 (m, 3H), 3.45-3.35 (m, 1H), 3.22-3.08 (m, 2H), 2.83-2.70 (m, 1H), 2.61-2.56 (m, 1H), 2.21-2.08 (m, 1H), 2.01-1.55 (m, 8H), 1.53-1.31 (m, 2H), 1.11-0.98 (m, 1H), 0.92-0.76 (m, 6H).

MS obsd. (ESI⁺) [(M+H)⁺]: 570.2.

### Example 74

### N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-cyclobutyl-2-oxo-ethyl]-2,2,2-trifluoro-acetamide

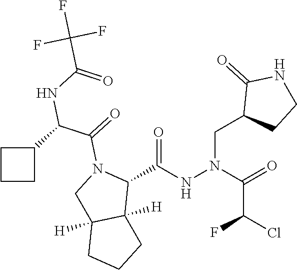 [SMILES: O=C1NCC[C@H]1CN(NC(=O)[C@@H]1[C@H]2CCC[C@H]2CN1C(=O)[C@@H](NC(=O)C(F)(F)F)C1CCC1)C(=O)[C@H](F)Cl]

The title compound was prepared in analogy to the procedure described for the preparation of Example 1, by using benzyl (3S,3aS,6aR)-2-[(2S)-2-amino-2-cyclobutyl-acetyl]-3,3a,4,5,6,6a-hexahydro-1H-cyclopenta[c]pyrrole-3-carboxylate; 2,2,2-trifluoroacetic acid (Intermediate 23) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-cyclobutyl-2-oxo-ethyl]-2,2,2-trifluoro-acetamide (Example 74) as a white solid.

¹H NMR (400 MHz, DMSO-d₆) δ: ppm 11.05 (s, 1H), 9.77 (d, J=7.2 Hz, 1H), 7.78 (s, 1H), 6.84 (d, J=50.4 Hz, 1H), 4.60-4.50 (m, 1H), 3.99 (d, J=5.2 Hz, 1H), 3.94-3.82 (m, 1H), 3.80-3.61 (m, 2H), 3.38 (dd, J=4.0, 14.0 Hz, 1H), 3.23-3.07 (m, 2H), 2.82-2.67 (m, 2H), 2.58-2.57 (m, 1H), 2.21-2.07 (m, 1H), 1.99-1.56 (m, 13H), 1.45-1.30 (m, 1H).

MS obsd. (ESI⁺) [(M+H)⁺]: 568.3.

### Example 75

### N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-methyl-2-oxo-ethyl]-2,2,2-trifluoro-acetamide

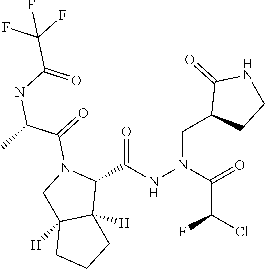 [SMILES: C[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 72, by using Cbz-Ala-OH (GL Biochem, CAS number: 142-20-7) instead of (S)-2-(((benzyloxy)carbonyl)amino)butanoic acid to afford N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-methyl-2-oxo-ethyl]-2,2,2-trifluoro-acetamide (Example 75) as a white solid.

¹H NMR (400 MHz, DMSO-d₆) δ: ppm 11.02 (s, 1H), 9.85 (d, J=6.4 Hz, 1H), 7.78 (s, 1H), 6.81 (d, J=50.4 Hz, 1H), 4.61-4.49 (m, 1H), 4.16-3.93 (m, 1H), 3.88-3.73 (m, 2H), 3.62-3.52 (m, 1H), 3.40-3.35 (m, 1H), 3.21-3.11 (m, 2H), 2.83-2.73 (m, 1H), 2.61-2.57 (m, 1H), 2.25-2.09 (m, 1H), 2.01-1.51 (m, 7H), 1.46-1.36 (m, 1H), 1.29-1.23 (m, 3H).

MS obsd. (ESI⁺) [(M+H)⁺]: 528.0.

### Example 76

### N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide

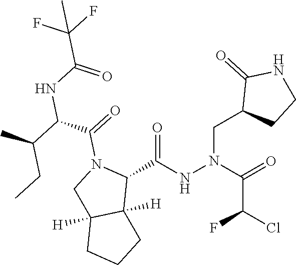 [SMILES: CC[C@@H](C)[C@H](NC(=O)C(C)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (3S,3aS,6aR)-2-[(2S,3R)-2-amino-3-methyl-pentanoyl]-3,3a,4,5,6,6a-hexahydro-1H-cyclopenta[c]pyrrole-3-carboxylate; 2,2,2-trifluoroacetic acid (Intermediate 22) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), 2,2-difluoropropionic acid and T₃P instead of TFAA to afford N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide (Example 76) as a white solid.

¹H NMR (400 MHz, DMSO-d₆) δ: ppm 11.10 (s, 1H), 8.77 (d, J=8.0 Hz, 1H), 7.78 (s, 1H), 6.91 (d, J=50.4 Hz, 1H), 4.41-4.31 (m, 1H), 3.99 (d, J=5.2 Hz, 1H), 3.86-3.68 (m, 3H), 3.39 (dd, J=14.0 Hz, 3.6 Hz, 1H), 3.22-3.12 (m, 2H), 2.79-2.66 (m, 2H), 2.22-2.11 (m, 1H), 1.99-1.55 (m, 11H), 1.50-1.35 (m, 2H), 1.12-1.00 (m, 1H), 0.91-0.77 (m, 6H).

MS obsd. (ESI⁺) [(M+H)⁺]: 566.3.

### Example 77

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2,2-trifluoro-acetamide

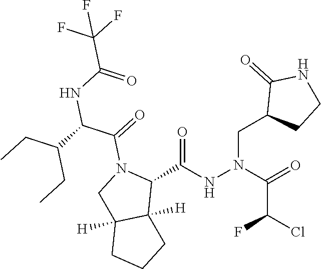 [SMILES: CCC(CC)[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2,2-trifluoro-acetamide (Example 77) as a white solid.

¹H NMR (400 MHz, DMSO-d₆) δ: ppm 11.10 (s, 1H), 9.88 (d, J=7.6 Hz 1H), 7.78 (s, 1H), 6.90 (d, J=50.4 Hz, 1H), 4.46 (t, J=8.4 Hz, 1H), 4.00 (d, J=6.0 Hz, 1H), 3.87-3.70 (m, 3H), 3.39 (dd, J=14.00, 4.0 Hz, 1H), 3.21-3.07 (m, 2H), 2.82-2.72 (m, 1H), 2.64-2.56 (m, 2H), 2.22-2.07 (m, 1H), 1.91-1.61 (m, 7H), 1.46-1.18 (m, 5H), 0.85-0.70 (m, 6H).

MS obsd. (ESI⁺) [(M+H)⁺]: 584.2.

### Example 78

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]-2,2,2-trifluoro-acetamide

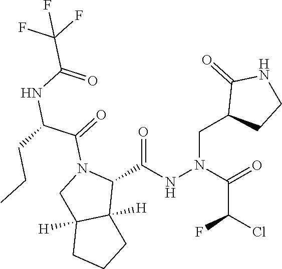 [SMILES: CCC[C@H](NC(=O)C(F)(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 3, by using (S)-2-(((benzyloxy)carbonyl)amino)pentanoic acid (Wuxiapptec, CAS number: 21691-44-1) instead of (2S)-2-(benzyloxycarbonylamino)-4-methyl-pentanoic acid and ethyl (3S,3aS,6aR)-1,2,3,3a,4,5,6,6a-octahydrocyclopenta[c]pyrrole-3-carboxylate; hydrochloride instead of methyl (1R,2S,5S)-6,6-dimethyl-3-azabicyclo[3.1.0]hexane-2-carboxylate; hydrochloride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]-2,2,2-trifluoro-acetamide (Example 78) as a white solid.

¹H NMR (400 MHz, DMSO-d₆) δ: ppm 11.06 (s, 1H), 9.82 (d, J=6.8 Hz, 1H), 7.78 (s, 1H), 6.85 (d, J=50.4 Hz, 1H), 4.55-4.43 (m, 1H), 4.13-3.99 (m, 1H), 3.95-3.74 (m, 2H), 3.70-3.60 (m, 1H), 3.39 (d, J=14.0, 4.0 Hz, 1H), 3.22-3.08 (m, 2H), 2.84-2.75 (m, 1H), 2.65-2.58 (m, 2H), 2.30-2.10 (m, 1H), 1.92-1.70 (m, 5H), 1.69-1.59 (m, 3H), 1.49-1.27 (m, 3H), 0.87 (t, J=7.2 Hz, 3H).

MS obsd. (ESI⁺) [(M+H)⁺]: 556.3.

### Example 79

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide

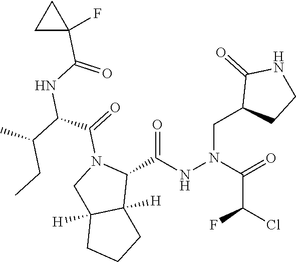 [SMILES: CC[C@H](C)[C@H](NC(=O)C1(F)CC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, 1-fluorocyclopropane-1-carboxylic acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide (Example 79) as a white solid.

¹H NMR (400 MHz, DMSO-d₆) δ: ppm 11.05 (s, 1H), 8.43 (d, J=7.2 Hz, 1H), 7.84-7.70 (m, 1H), 7.07-6.80 (m, 1H), 4.30 (t, J=8.6 Hz, 1H), 3.97 (d, J=6.0 Hz, 1H), 3.88-3.78 (m, 2H), 3.78-3.70 (m, 1H), 3.39 (dd, J=14.0 Hz, 4.0 Hz, 1H), 3.22-3.10 (m, 2H), 2.78-2.68 (m, 1H), 2.60-2.52 (m, 2H), 2.11-2.20 (m, 1H), 1.98-1.90 (m, 1H), 1.87-1.68 (m, 5H), 1.66-1.56 (m, 1H), 1.54-1.44 (m, 1H), 1.41-1.33 (m, 1H), 1.33-1.26 (m, 2H), 1.23-1.03 (m, 3H), 0.88-0.78 (m, 6H).

MS obsd. (ESI⁺) [(M+H)⁺]: 560.2.

### Example 80

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]benzamide

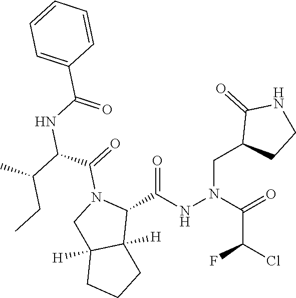 [SMILES: CC[C@H](C)[C@H](NC(=O)c1ccccc1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine, benzoic acid and T₃P instead of acetic anhydride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]benzamide (Example 80) as a white solid.

¹H NMR (400 MHz, DMSO-d₆) δ: ppm 11.04 (s, 1H), 8.72 (d, J=8.0 Hz, 1H), 7.93-7.87 (m, 2H), 7.77 (s, 1H), 7.56-7.50 (m, 1H), 7.48-7.42 (m, 2H), 7.08-6.80 (m, 1H), 4.46-4.38 (m, 1H), 4.09-4.00 (m, 1H), 3.97 (d, J=6.0 Hz, 1H), 3.93-3.85 (m, 1H), 3.79-3.69 (m, 1H), 3.42 (dd, J=14.0 Hz, 4.0 Hz, 1H), 3.22-3.10 (m, 2H), 2.81-2.72 (m, 1H), 2.62-2.52 (m, 2H), 2.20-2.10 (m, 1H), 2.03-1.93 (m, 1H), 1.93-1.82 (m, 1H), 1.82-1.68 (m, 4H), 1.68-1.52 (m, 2H), 1.48-1.37 (m, 1H), 1.24-1.11 (m, 1H), 0.90 (d, J=6.8 Hz, 3H), 0.84 (t, J=7.2 Hz, 3H).

MS obsd. (ESI⁺) [(M+H)⁺]: 578.2.

### Example 81

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]acetamide

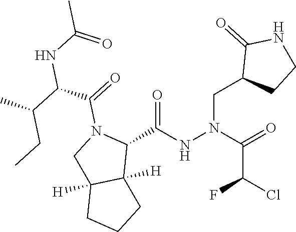 [SMILES: CC[C@H](C)[C@H](NC(C)=O)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (2S,3S)-2-(benzyloxycarbonylamino)-3-methyl-pentanoic acid instead of Cbz-L-tert-leucine to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]acetamide (Example 81) as a white solid.

¹H NMR (400 MHz, DMSO-d₆) δ: ppm 11.03 (s, 1H), 8.23 (d, J=8.4 Hz, 1H), 7.77 (s, 1H), 6.99 (d, J=50.4 Hz, 1H), 4.26-4.21 (m, 1H), 3.93 (d, J=6.0 Hz, 1H), 3.85-3.70 (m, 3H), 3.39 (dd, J=14.0, 4.4 Hz, 1H), 3.22-3.10 (m, 2H), 2.79-2.69 (m, 1H), 2.60-2.55 (m, 1H), 2.21-2.08 (m, 1H), 1.87-1.78 (m, 5H), 1.77-1.56 (m, 6H), 1.54-1.44 (m, 1H), 1.41-1.34 (m, 1H), 1.13-1.03 (m, 1H), 0.85-0.78 (m, 6H).

MS obsd. (ESI⁺) [(M+H)⁺]: 516.3.

### Example 82

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide

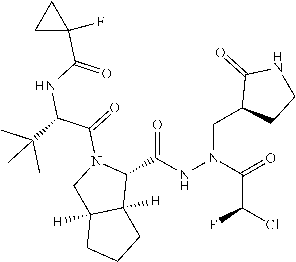 [SMILES: CC(C)(C)[C@H](NC(=O)C1(F)CC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using 1-fluorocyclopropane-1-carboxylic acid and T₃P instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide (Example 82) as a white solid.

¹H NMR (400 MHz, DMSO-d₆) δ: ppm 11.10 (s, 1H), 7.76 (s, 1H), 7.40 (d, J=7.6 Hz, 1H), 6.97 (d, J=50.4 Hz, 1H), 4.51 (d, J=8.8 Hz, 1H), 3.99 (d, J=6.0 Hz, 1H), 3.93-3.83 (m, 1H), 3.78-3.63 (m, 2H), 3.40 (dd, J=14.0 Hz, 7.6 Hz, 1H), 3.22-3.10 (m, 2H), 2.78-2.69 (m, 1H), 2.60-2.55 (m, 2H), 2.25-2.10 (m, 1H), 1.90-1.70 (m, 5H), 1.66-1.56 (m, 1H), 1.40-1.30 (m, 3H), 1.25-1.12 (m, 2H), 0.97 (s, 9H).

MS obsd. (ESI⁺) [(M+H)⁺]: 560.3.

### Example 83

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide

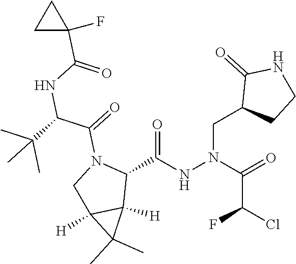 [SMILES: CC(C)(C)[C@H](NC(=O)C1(F)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 29, by using 1-fluorocyclopropane-1-carboxylic acid and T₃P instead of acetic anhydride, methyl (1R,2S,5S)-6,6-dimethyl-3-azabicyclo[3.1.0]hexane-2-carboxylate; hydrochloride instead of methyl (3S,3aS,6aR)-1,2,3,3a,4,5,6,6a-octahydrocyclopenta[c]pyrrole-3-carboxylate; hydrochloride to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide (Example 83) as a white solid.

¹H NMR (400 MHz, DMSO-d₆) δ: ppm 11.10 (s, 1H), 7.76 (s, 1H), 7.40 (d, J=7.6 Hz, 1H), 6.97 (d, J=50.4 Hz, 1H), 4.51 (d, J=8.8 Hz, 1H), 3.99 (d, J=6.0 Hz, 1H), 3.93-3.83 (m, 1H), 3.78-3.63 (m, 2H), 3.40 (dd, J=14.0 Hz, 7.6 Hz, 1H), 3.22-3.10 (m, 2H), 2.78-2.69 (m, 1H), 2.60-2.55 (m, 2H), 2.25-2.10 (m, 1H), 1.90-1.70 (m, 5H), 1.66-1.56 (m, 1H), 1.40-1.30 (m, 3H), 1.25-1.12 (m, 2H), 0.97 (s, 9H).

MS obsd. (ESI⁺) [(M+H)⁺]: 560.3.

### Example 84

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2,2-trifluoro-acetamide

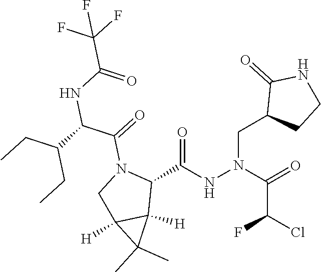 [SMILES: CCC(CC)[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 64, by using (2S)-2-(tert-butoxycarbonylamino)-3-ethyl-pentanoic acid (KaiXin Biological, CAS number: 35264-04-1) instead of Boc-L-isoleucine to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2,2-trifluoro-acetamide (Example 84) as a white solid.

¹H NMR (400 MHz, DMSO-d₆) δ: ppm 11.18 (s, 1H), 9.94 (d, J=8.0 Hz, 1H), 7.77 (s, 1H), 6.80 (d, J=50.4 Hz, 1H), 4.42-4.34 (m, 1H), 4.17 (s, 1H), 3.95-3.75 (m, 3H), 3.48-3.40 (dd, J=14.0 Hz, 4.4 Hz, 1H), 3.21-3.10 (m, 2H), 2.63-2.57 (m, 1H), 2.25-2.10 (m, 1H), 1.95-1.85 (m, 1H), 1.83-1.72 (m, 1H), 1.68-1.61 (m, 1H), 1.57-1.52 (m, 1H), 1.48-1.38 (m, 1H), 1.35-1.19 (m, 3H), 1.06 (s, 3H), 0.89 (s, 3H), 0.83-0.72 (m, 6H).

MS obsd. (ESI⁺) [(M+H)⁺]: 584.1.

### Example 85

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-propanamide

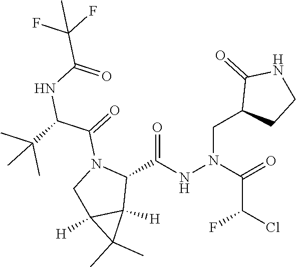 [SMILES: CC(F)(F)C(=O)N[C@H](C(=O)N1C[C@H]2[C@@H]([C@H]1C(=O)NN(C[C@@H]1CCNC1=O)C(=O)[C@@H](F)Cl)C2(C)C)C(C)(C)C]

The title compound was prepared in analogy to the procedure described for the preparation of Example 29, by using 2,2-difluoropropionic acid and T₃P instead of acetic anhydride, methyl (1R,2S,5S)-6,6-dimethyl-3-azabicyclo[3.1.0]hexane-2-carboxylate; hydrochloride instead of methyl (3S,3aS,6aR)-1,2,3,3a,4,5,6,6a-octahydrocyclopenta[c]pyrrole-3-carboxylate; hydrochloride and (2S)-2-chloro-2-fluoro-acetyl chloride (Intermediate 28) instead of (2R)-2-chloro-2-fluoro-acetyl chloride (Intermediate 10) to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-propanamide (Example 85) as a white solid.

¹H NMR (400 MHz, DMSO-d₆) δ: ppm 11.10-10.85 (m, 1H), 8.26 (d, J=8.8 Hz, 1H), 7.80-7.67 (m, 1H), 7.15-6.55 (m, 1H), 4.43-4.37 (m, 1H), 4.25-4.12 (m, 1H), 3.95-3.88 (m, 1H), 3.82-3.67 (m, 2H), 3.53 (dd, J=14.0 Hz, 4.4 Hz, 1H), 3.20-2.95 (m, 2H), 2.53-2.52 (m, 1H), 2.24-2.10 (m, 1H), 1.82-1.55 (m, 6H), 1.06 (s, 3H), 1.01-0.95 (m, 9H), 0.88 (s, 3H).

MS obsd. (ESI⁺) [(M+H)⁺]: 566.5.

### Example 86

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2,2-trifluoro-acetamide

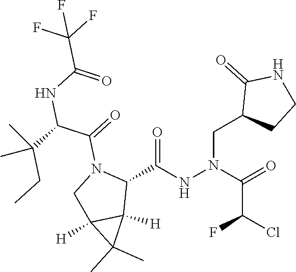 [SMILES: CCC(C)(C)[C@H](NC(=O)C(F)(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 64, by using (2S)-2-(tert-butoxycarbonylamino)-3,3-dimethyl-pentanoic acid (Intermediate 25) instead of Boc-L-isoleucine to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2,2-trifluoro-acetamide (Example 86) as a white solid.

¹H NMR (400 MHz, CDCl₃) δ: ppm 10.01-9.95 (m, 1H), 7.04-6.98 (m, 1H), 6.69-6.57 (m, 1H), 6.07-5.99 (m, 1H), 4.65 (d, J=8.8 Hz, 1H), 4.44 (s, 2H), 4.06-4.02 (m, 1H), 3.88 (d, J=9.6 Hz, 1H), 3.43 (s, 2H), 2.91-2.80 (m, 2H), 2.42-2.39 (m, 1H), 1.83-1.78 (m, 1H), 1.62 (s, 1H), 1.36-1.31 (m, 3H), 1.07 (s, 3H), 0.98 (d, J=12.8 Hz, 6H), 0.91-0.86 (m, 6H).

MS obsd. (ESI⁺) [(M+H)⁺]: 584.2.

### Example 87

### 3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide

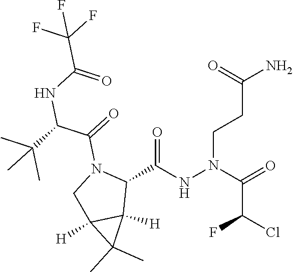 [SMILES: CC(C)(C)[C@H](NC(=O)C(F)(F)F)C(=O)N1C[C@H]2[C@@H]([C@H]1C(=O)NN(CCC(N)=O)C(=O)[C@H](F)Cl)C2(C)C]

### Step 1: Preparation of methyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate

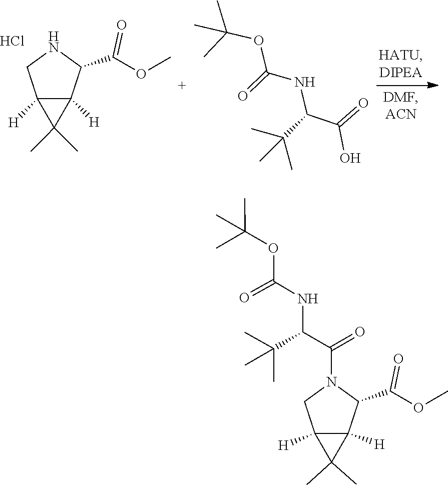

To a solution of methyl (1R,2S,5S)-6,6-dimethyl-3-azabicyclo[3.1.0]hexane-2-carboxylate; hydrochloride (50.0 g, 243.1 mmol) in a mixed solution of DMF (95 mL) and MeCN (850 mL) was added HATU (101.6 g, 267.41 mmol). Then DIPEA (94.3 g, 729.29 mmol) was added at 0° C. The reaction was allowed to warm to 25° C. and stirred for 2 h. The reaction solution was concentered and the residue was diluted with EtOAc (500 mL) and washed with water (200 mL). The aqueous phase was extracted with EtOAc (100 mL×2). The combined organic layers were washed with 1 N HCl (100 mL), an aqueous solution of K₂CO₃ (100 mL, 5%) and brine (100 mL). The organic phase was dried over Na₂SO₄, filtered and concentrated in vacuum. The residue was purified by silica gel column eluted with PE to PE/EtOAc=10/1 to afford methyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate (80.0 g) as colorless oil.

MS obsd. (ESI⁺) [(M+Na)⁺]: 405.3.

### Step 2: Preparation of (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid

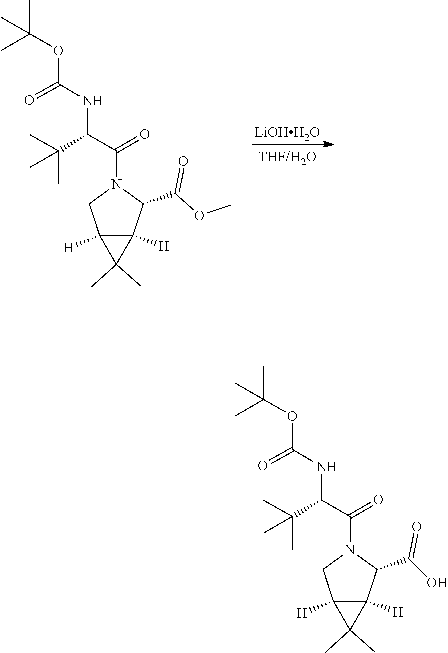 [SMILES: COC(=O)[C@@H]1[C@@H]2[C@H](CN1C(=O)[C@@H](NC(=O)OC(C)(C)C)C(C)(C)C)C2(C)C ; "LiOH·H2O" ; CC(C)(C)OC(=O)N[C@H](C(=O)N1C[C@H]2[C@@H]([C@H]1C(=O)O)C2(C)C)C(C)(C)C]

To a solution of methyl (1R,2S,5S)-3-[(2S)-2-(tert-butoxycarbonylamino)-3,3-dimethyl-butanoyl]-6,6-dimethyl-3-azabicyclo[3.1.0]hexane-2-carboxylate (72.0 g, 188.24 mmol) in THF (400 mL) was added a solution of LiOH·H₂O (23.7 g, 564.72 mmol) in water (100 mL). The solution was stirred at 25° C. for 2 h. The reaction was acidified with 1 N HCl to pH=5. The resulting solution was concentrated to remove most of the THF. The residue was diluted with H₂O (200 mL) and extracted with EtOAc (150 mL×3). The combined organic layers were washed with brine (200 mL), dried over Na₂SO₄, filtered and concentrated to afford (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (68.0 g) as a white solid.

MS obsd. (ESI⁺) [(M+H)⁺]: 369.3.

### Step 3: Preparation of (1R,2S,5S)-3-[(2S)-2-amino-3,3-dimethyl-butanoyl]-6,6-dimethyl-3-azabicyclo[3.1.0]hexane-2-carboxylic acid; hydrochloride

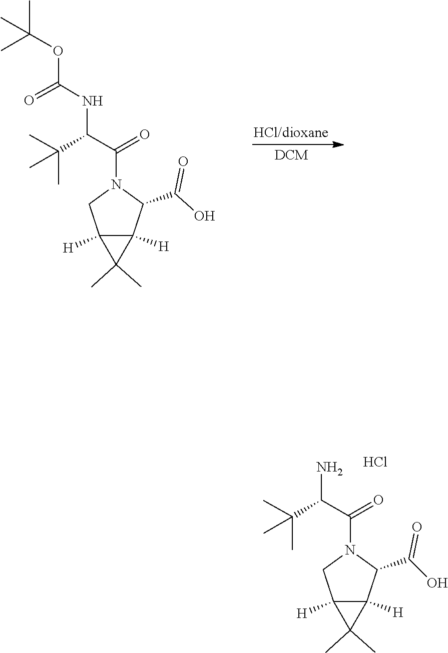

To a solution of (1R,2S,5S)-3-[(2S)-2-(tert-butoxycarbonylamino)-3,3-dimethyl-butanoyl]-6,6-dimethyl-3-azabicyclo[3.1.0]hexane-2-carboxylic acid (68.0 g, 184.55 mmol) in DCM (180 mL) was added HCl/dioxane (230.68 mL, 4 N). The mixture was stirred at 25° C. for 4 h. The reaction was concentrated to afford (1R,2S,5S)-3-[(2S)-2-amino-3,3-dimethyl-butanoyl]-6,6-dimethyl-3-azabicyclo[3.1.0]hexane-2-carboxylic acid; hydrochloride (56.0 g) as a white solid.

MS obsd. (ESI⁺) [(M+H)⁺]: 269.3.

### Step 4: Preparation of (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid

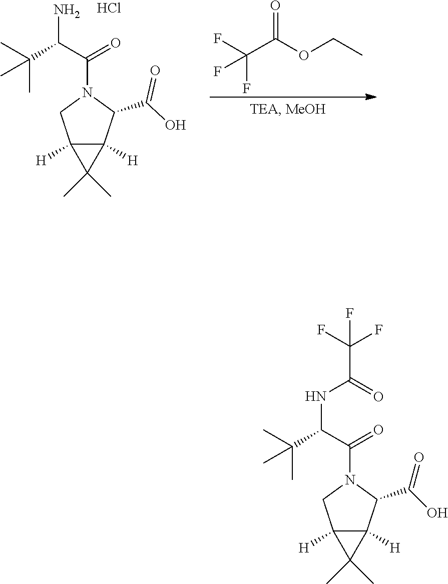

To a solution of (1R,2S,5S)-3-[(2S)-2-amino-3,3-dimethyl-butanoyl]-6,6-dimethyl-3-azabicyclo[3.1.0]hexane-2-carboxylic acid hydrochloride (56.0 g, 183.72 mmol) in methanol (180 mL) was added triethylamine (153.6 mL, 1102.33 mmol) and ethyl trifluoroacetate (70.5 g, 496.05 mmol) dropwise. The reaction was warmed to 50° C. and stirred for 16 h. The mixture was concentrated to remove MeOH. The residue was dissolved in EtOAc (400 mL) and washed with 0.5 N HCl (300 mL). The aqueous phase was extracted with EtOAc (300 mL×3). The combined organic phase was washed with brine (100 mL), dried over Na₂SO₄, filtered and concentrated. The residue was triturated in PE (300 mL) for 10 min. The suspension was filtered. The cake was washed with PE (50 mL), collected and dried in vacuum to afford (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (65.0 g) as a white solid.

MS obsd. (ESI⁺) [(M+H)+]: 365.3.

### Step 5: Preparation of benzyl N-(3-amino-3-oxo-propyl)-N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]carbamate

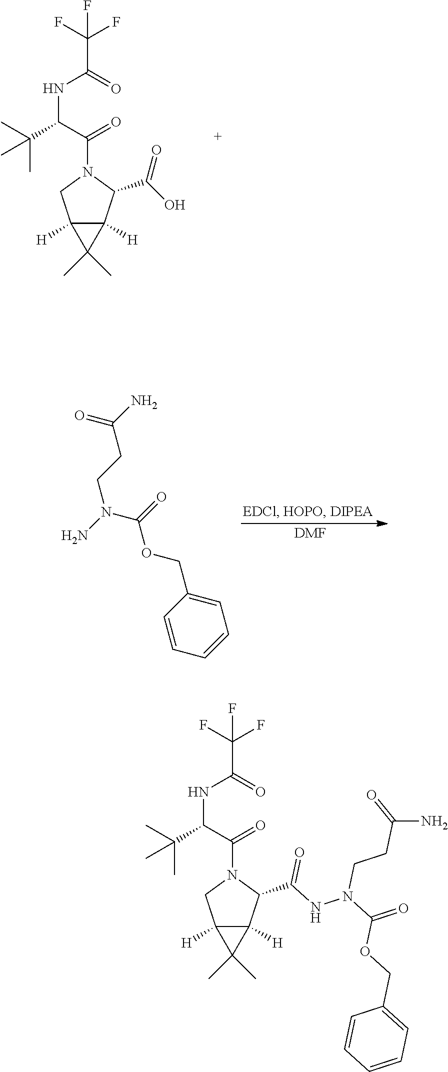

To a solution of (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (540 mg, 1.48 mmol) in DMF (5 mL) was added DIPEA (1149 mg, 8.89 mmol), EDCI (341 mg, 1.78 mmol), HOPO (198 mg, 1.78 mmol) and benzyl N-amino-N-(3-amino-3-oxo-propyl)carbamate; 2,2,2-trifluoroacetic acid (520.62 mg, 1.48 mmol, Intermediate 26) at 0° C. The reaction mixture was stirred at 20° C. for 12 h and the mixture was purified by prep-HPLC (TFA condition, column: Phenomenex Luna C18 150*25 mm*10 um; mobile phase: [water(TFA)-ACN]; B %: 42%-72%, 1 min) to afford benzyl N-(3-amino-3-oxo-propyl)-N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]carbamate (500 mg) as colorless oil.

MS obsd. (ESI⁺) [(M+H)⁺]: 584.3.

### Step 6: Preparation of 3-[2-[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]hydrazino]propanamide

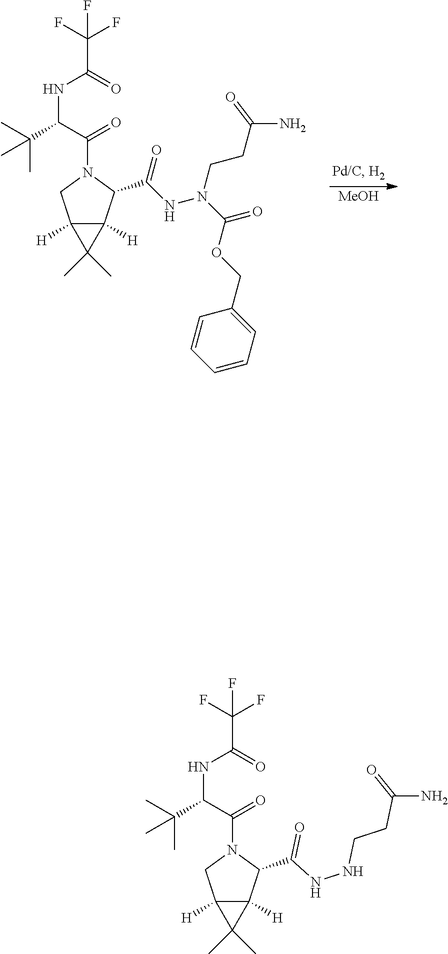 [SMILES: "MeOH" ; CC(C)(C)[C@H](NC(=O)C(F)(F)F)C(=O)N1C[C@H]2[C@@H]([C@H]1C(=O)NN(CCC(N)=O)C(=O)OCc1ccccc1)C2(C)C ; CC(C)(C)[C@H](NC(=O)C(F)(F)F)C(=O)N1C[C@H]2[C@@H]([C@H]1C(=O)NNCCC(N)=O)C2(C)C]

To a solution of benzyl N-(3-amino-3-oxo-propyl)-N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]carbamate (500 mg, 0.860 mmol) in methanol (3 mL) was added Pd/C (50 mg, 10% purity). The reaction mixture was degassed under vacuum and purged H₂ for 3 times. The resulting mixture was stirred at 25° C. for 2 h under a H₂ balloon. The mixture was filtered through a pad of celite and washed with EtOAc (10 mL×3). The filtrate was concentrated in vacuum to afford 3-[2-[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]hydrazino]propanamide (480 mg) as colorless oil.

MS obsd. (ESI⁺) [(M+H)⁺]: 450.2.

### Step 7: Preparation of 3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide (Example 87)

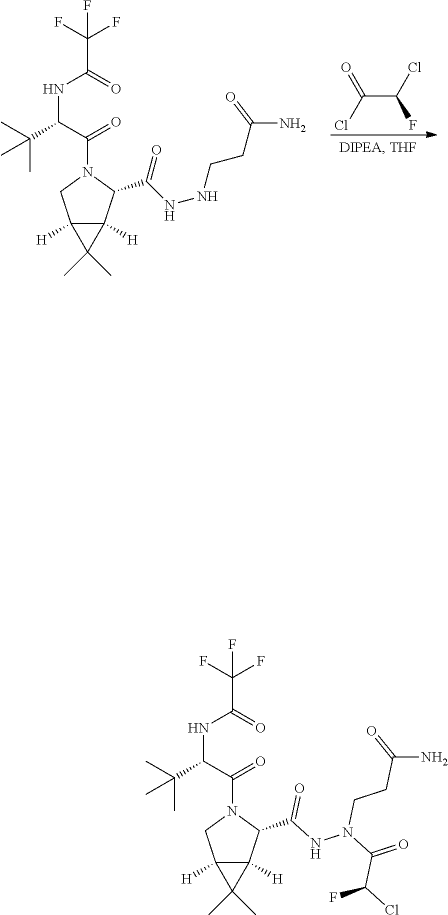 [SMILES: CC(C)(C)[C@H](NC(=O)C(F)(F)F)C(=O)N1C[C@H]2[C@@H]([C@H]1C(=O)NNCCC(N)=O)C2(C)C ; O=C(Cl)[C@H](F)Cl ; CC(C)(C)[C@H](NC(=O)C(F)(F)F)C(=O)N1C[C@H]2[C@@H]([C@H]1C(=O)NN(CCC(N)=O)C(=O)[C@H](F)Cl)C2(C)C]

To a solution of 3-[2-[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]hydrazino]propanamide (292 mg, 0.6 mmol) in THF (70 mL) was added DIPEA (840 mg, 6.5 mmol) and a solution of (2R)-2-chloro-2-fluoro-acetyl chloride (428.0 mg, 1.31 mmol, Intermediate 9) in THF (17 mL) successively at −20° C. over 10 min. The mixture was stirred at 0° C. for 30 min. The reaction mixture was quenched with MeOH (6 mL) and acidified with 4 N HCl/dioxane to pH=5 at 0° C. The mixture was concentrated in vacuum and the residue was purified by prep-HPLC (Condition: water (0.1% TFA)-ACN, Column:3_Phenomenex Luna C18 75*30 mm*3 um, 39-59% MeCN in H₂O, FlowRate(ml/min):25) to afford 3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide (62 mg, Example 87) as a white solid.

¹H NMR (400 MHz, CDCl₃) δ: ppm 9.61 (br. s, 1H), 7.04 (d, J=8.8 Hz, 1H), 6.70-6.57 (m, 1H), 6.39-5.98 (m, 2H), 4.56 (d, J=9.2 Hz, 1H), 4.37-4.17 (m, 2H), 4.05 (dd, J=10.0, 5.6 Hz, 1H), 3.87 (d, J=10.4 Hz, 1H), 3.69-3.43 (m, 1H), 2.71-2.59 (m, 2H), 1.67-1.64 (m, 1H), 1.46 (d, J=7.2 Hz, 1H), 1.09 (s, 3H), 1.03 (s, 9H), 0.92 (s, 3H).

MS obsd. (ESI⁺) [(M+H)⁺]: 544.2.

### Example 88a and Example 88b

### (3S,3aS,6aR)—N′-[(2R)-2-chloro-2-fluoro-acetyl]-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide and (3R,3aR,6aS)—N′-[(2R)-2-chloro-2-fluoro-acetyl]-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide

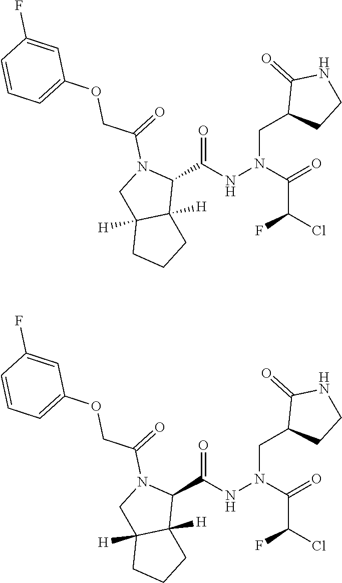 [SMILES: O=C1NCC[C@H]1CN(NC(=O)[C@@H]1[C@H]2CCC[C@H]2CN1C(=O)COc1cccc(F)c1)C(=O)[C@H](F)Cl ; O=C1NCC[C@H]1CN(NC(=O)[C@H]1[C@@H]2CCC[C@@H]2CN1C(=O)COc1cccc(F)c1)C(=O)[C@H](F)Cl]

### Step 1: Preparation of trans-methyl 2-[2-(3-fluorophenoxy)acetyl]-3,3a,4,5,6,6a-hexahydro-1H-cyclopenta[c]pyrrole-3-carboxylate

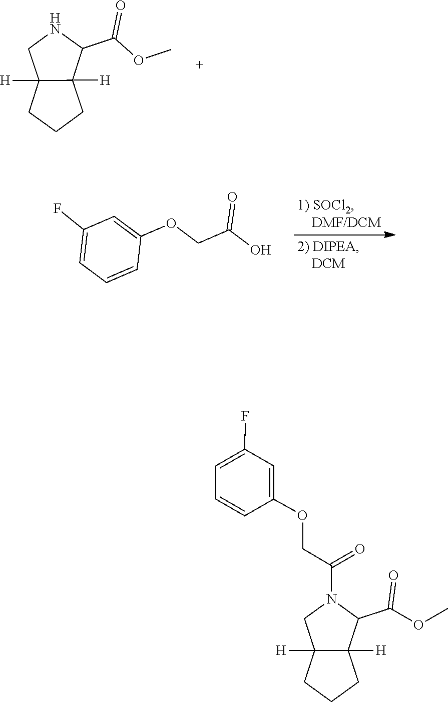 [SMILES: COC(=O)C1NCC2CCCC21 ; O=C(O)COc1cccc(F)c1 ; COC(=O)C1C2CCCC2CN1C(=O)COc1cccc(F)c1]

To a solution of (3-fluoro-phenoxy)-acetic acid (300 mg, 1.76 mmol) in DCM (5 mL) was added SOCl₂ (416 mg, 3.53 mmol) and DMF (6 mg, 0.090 mmol). The mixture was stirred at 60° C. for 1 h. The mixture was concentrated in vacuum to afford the crude 2-(3-fluorophenoxy)acetyl chloride (330 mg) as yellow oil. Then the crude 2-(3-fluorophenoxy)acetyl chloride and DIPEA (764 mg, 5.91 mmol) was added to a solution of trans-methyl 1,2,3,3a,4,5,6,6a-octahydrocyclopenta[c]pyrrole-3-carboxylate (200 mg, 1.18 mmol, Intermediate 27) in DCM (5 mL) at 0° C. The reaction mixture was stirred at 25° C. for 2 h. The mixture was concentrated in vacuum and the residue was purified by reverse flash (condition: 120 g Flash Column Welch Ultimate XB_C18 20-40 μm; 120 A, water (0.1% HCl)-ACN, 0˜55%, 60 ml/min) to afford trans-methyl 2-[2-(3-fluorophenoxy)acetyl]-3,3a,4,5,6,6a-hexahydro-1H-cyclopenta[c]pyrrole-3-carboxylate (351 mg) as a white solid.

MS obsd. (ESI⁺) [(M+H)⁺]: 322.2.

### Step 2: Preparation of trans-2-[2-(3-fluorophenoxy)acetyl]-3,3a,4,5,6,6a-hexahydro-1H-cyclopenta[c]pyrrole-3-carboxylic acid

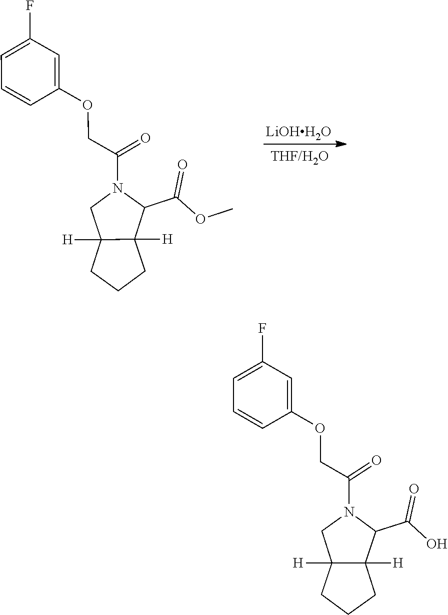

To a solution of trans-methyl 2-[2-(3-fluorophenoxy)acetyl]-3,3a,4,5,6,6a-hexahydro-1H-cyclopenta[c]pyrrole-3-carboxylate (350 mg, 1.09 mmol) in THF (5 mL) was added the solution of LiOH·H₂O (91 mg, 2.18 mmol) in water (2 mL). The mixture was stirred at 25° C. for 2 h. The mixture was poured into 1 N HCl (60 mL) and extracted with EtOAc (40 mL×3). The organic phase was washed with brine (40 mL), dried over Na₂SO₄, filtered and concentrated. The residue was purified by reverse flash chromatography (condition: 120 g Flash Column Welch Ultimate XB_C18 20-40 μm; 120 A, water (0.1% HCl)-ACN, 0˜55%, 60 mL/min) to afford trans-2-[2-(3-fluorophenoxy)acetyl]-3,3a,4,5,6,6a-hexahydro-1H-cyclopenta[c]pyrrole-3-carboxylic acid (290 mg) as a yellow solid.

MS obsd. (ESI⁺) [(M+H)⁺]: 308.1.

### Step 3: Preparation of trans-tert-butyl N-[[2-[2-(3-fluorophenoxy)acetyl]-3,3a,4,5,6,6a-hexahydro-1H-cyclopenta[c]pyrrole-3-carbonyl]amino]-N-[[(3S)-2-oxopyrrolidin-3-yl]methyl]carbamate

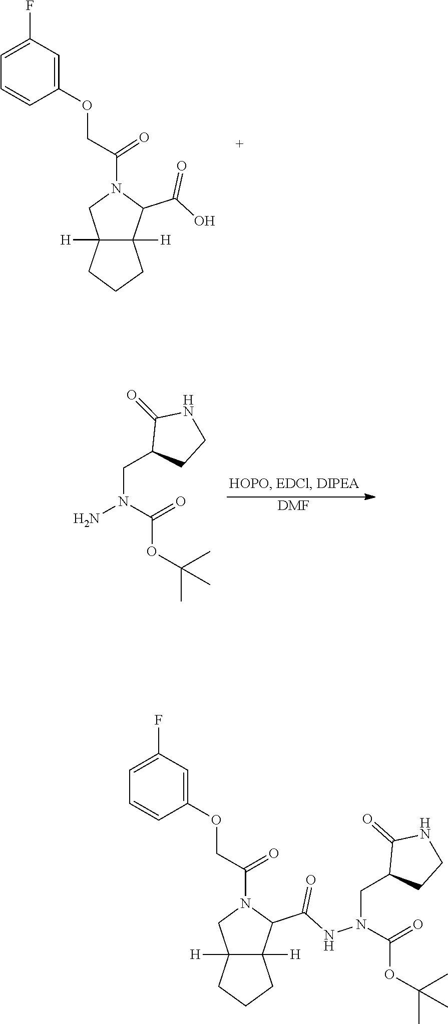

To a solution of trans-2-[2-(3-fluorophenoxy)acetyl]-3,3a,4,5,6,6a-hexahydro-1H-cyclopenta[c]pyrrole-3-carboxylic acid (250 mg, 0.810 mmol) in DMF (2 mL) was added HOPO (108 mg, 0.980 mmol), EDCI (187 mg, 0.980 mmol), DIPEA (315 mg, 2.4 mmol) and tert-butyl N-amino-N-[[(3S)-2-oxopyrrolidin-3-yl]methyl]carbamate (187 mg, 0.810 mmol) at 0° C. The mixture was stirred at 30° C. for 12 h. The mixture was purified by reverse flash chromatography (condition: 120 g Flash Column Welch Ultimate XB_C18 20-40 μm; 120 A, water (0.1% TFA)-ACN, 0˜55%, 60 ml/min) to afford trans-tert-butyl N-[[2-[2-(3-fluorophenoxy)acetyl]-3,3a,4,5,6,6a-hexahydro-1H-cyclopenta[c]pyrrole-3-carbonyl]amino]-N-[[(3S)-2-oxopyrrolidin-3-yl]methyl]carbamate (250 mg) as a white solid.

MS obsd. (ESI⁺) [(M+H)⁺]: 519.4.

### Step 4: Preparation of trans-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide

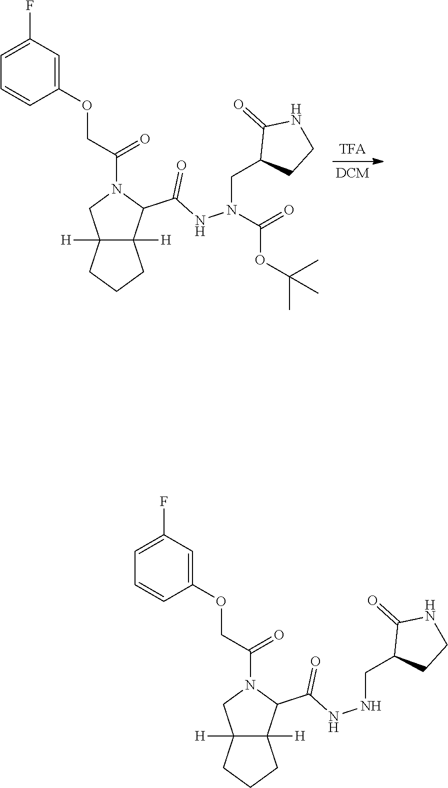 [SMILES: CC(C)(C)OC(=O)N(C[C@@H]1CCNC1=O)NC(=O)C1C2CCCC2CN1C(=O)COc1cccc(F)c1 ; O=C(NNC[C@@H]1CCNC1=O)C1C2CCCC2CN1C(=O)COc1cccc(F)c1]

To a solution of trans-tert-butyl N-[[2-[2-(3-fluorophenoxy)acetyl]-3,3a,4,5,6,6a-hexahydro-1H-cyclopenta[c]pyrrole-3-carbonyl]amino]-N-[[(3S)-2-oxopyrrolidin-3-yl]methyl]carbamate (250 mg, 0.480 mmol) in DCM (5 mL) was added TFA (10.0 mL). The mixture was stirred at 25° C. for 2 h. The mixture was concentrated and the residue was purified by reverse flash chromatography (condition: 120 g Flash Column Welch Ultimate XB_C18 20-40 μm; 120 A, water (0.1% HCl)-ACN, 0˜55%, 60 mL/min) to afford trans-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide (160 mg) as a white solid.

### Step 5: Preparation of (3S,3aS,6aR)—N′-[(2R)-2-chloro-2-fluoro-acetyl]-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide and (3R,3aR,6aS)—N′-[(2R)-2-chloro-2-fluoro-acetyl]-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide (Example 88a and 88b)

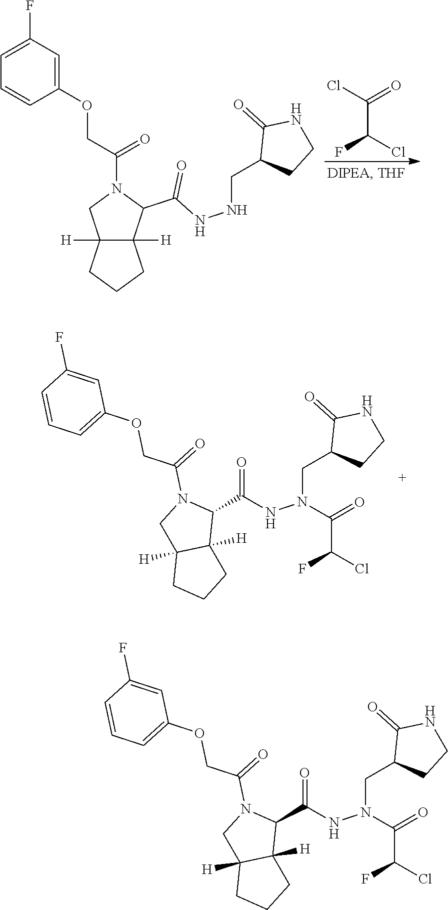

To a solution of trans-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide (160 mg, 0.380 mmol) in THF (35 mL) was added DIPEA (741 mg, 5.74 mmol) at 0° C. Then (2R)-2-chloro-2-fluoro-acetyl chloride (10 mg, 0.760 mmol in THF (5 mL) was added at 0° C. The resulting mixture was stirred at 0° C. for 30 min. The reaction mixture was quenched with MeOH (10 mL) and acidified by 4 N HCl/dioxane to pH=5 at −10° C. Then the mixture was concentrated in vacuum at 30° C. The residue was purified by reverse flash chromatography (condition: 120 g Flash Column Welch Ultimate XB_C18 20-40 μm; 120 A, water (0.1% FA)-ACN, 0˜55%, 60 mL/min) to afford 80 mg of two isomers. These two isomers was purified by prep-SFC (Instrument ACSWH-PREP-SFC-D Method: Column DAICEL CHIRALPAK AD (250 mm*30 mm, 10 um); Condition Neu-ETOH Begin B 40 End B 40; Gradient Time (min) 4.5; 100% B Hold Time (min) Flow Rate (mL/min) 70; Injections 50) to afford Example 88a (23 mg, retention time=1.094 min) and Example 88b (48 mg, retention time=2.551 min) as a white solid.

### Example 88a

¹H NMR (400 MHz, DMSO-d₆) δ: ppm 10.82-10.58 (m, 1H), 7.84-7.70 (m, 1H), 7.33-7.23 (m, 1H), 7.05-6.52 (m, 4H), 4.94-4.83 (m, 1H), 4.80-4.40 (m, 1H), 4.13-3.92 (m, 1H), 3.85-3.70 (m, 1H), 3.45 (dd, J=10.4 Hz, 4.4 Hz, 1H), 3.18-3.04 (m, 2H), 2.88-2.77 (m, 1H), 2.70-2.52 (m, 3H), 2.24-2.02 (m, 2H), 1.94-1.85 (m, 1H), 1.85-1.76 (m, 1H), 1.76-1.66 (m, 2H), 1.65-1.54 (m, 2H), 1.53-1.43 (m, 1H).

MS obsd. (ESI⁺) [(M+H)⁺]: 513.2.

### Example 88b

¹H NMR (400 MHz, DMSO-d₆) δ: ppm 11.02 (s, 1H), 7.82-7.66 (m, 1H), 7.33-7.22 (m, 1H), 6.85-6.65 (m, 4H), 4.93-4.70 (m, 2H), 4.15-4.01 (m, 1H), 3.96-3.68 (m, 2H), 3.47 (dd, J=10.4 Hz, 4.0 Hz, 1H), 3.40-3.33 (m, 1H), 3.21-2.99 (m, 2H), 2.87-2.74 (m, 1H), 2.61-2.52 (m, 2H), 2.21-2.08 (m, 1H), 1.89-1.55 (m, 6H), 1.53-1.41 (m, 1H).

MS obsd. (ESI⁺) [(M+H)⁺]: 513.2.

### Example 89a and Example 89b

### (3S,3aS,6aR)—N′-[(2R)-2-chloro-2-fluoro-acetyl]-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide and (3R,3aR,6aS)—N′-[(2R)-2-chloro-2-fluoro-acetyl]-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide

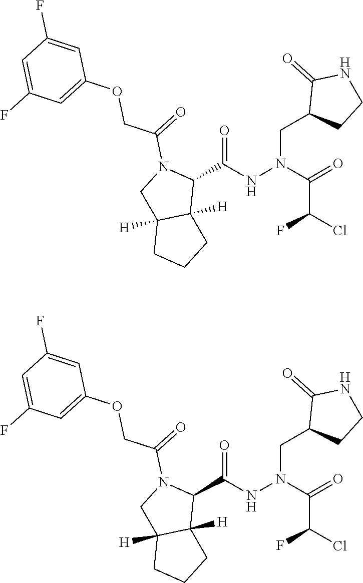 [SMILES: O=C1NCC[C@H]1CN(NC(=O)[C@@H]1[C@H]2CCC[C@H]2CN1C(=O)COc1cc(F)cc(F)c1)C(=O)[C@H](F)Cl ; O=C1NCC[C@H]1CN(NC(=O)[C@H]1[C@@H]2CCC[C@@H]2CN1C(=O)COc1cc(F)cc(F)c1)C(=O)[C@H](F)Cl]

### Step 1: Preparation of trans-methyl 2-(2-chloroacetyl)-3,3a,4,5,6,6a-hexahydro-1H-cyclopenta[c]pyrrole-3-carboxylate

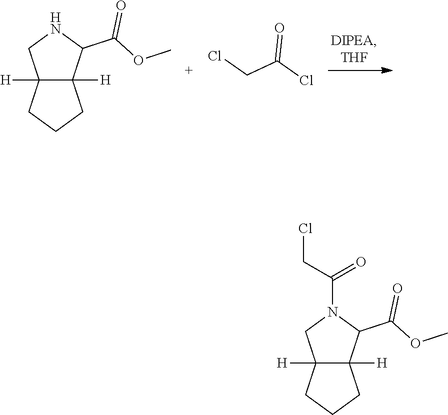 [SMILES: COC(=O)C1NCC2CCCC21 ; O=C(Cl)CCl ; COC(=O)C1C2CCCC2CN1C(=O)CCl]

To a solution of methyl trans-1,2,3,3a,4,5,6,6a-octahydrocyclopenta[c]pyrrole-3-carboxylate (200.0 mg, 1.18 mmol, Intermediate 27) in THF (8 mL) was added DIPEA (1.22 g, 9.46 mmol) and chloroacetyl chloride (267 mg, 2.36 mmol) at 0° C. The mixture was stirred at 0° C. for 30 min. The mixture was diluted with water (30 mL) and extracted with EtOAc (30 mL×3). The combined organic phase were dried over Na₂SO₄, filtered and concentrated and the residue was purified by silica gel column eluted with PE to PE/EtOAc=6/1 to afford trans-methyl 2-(2-chloroacetyl)-3,3a,4,5,6,6a-hexahydro-1H-cyclopenta[c]pyrrole-3-carboxylate (272 mg) as light yellow oil.

MS obsd. (ESI⁺) [(M+H)⁺]: 245.9.

### Step 2: Preparation of trans-methyl 2-[2-(3,5-difluorophenoxy)acetyl]-3,3a,4,5,6,6a-hexahydro-1H-cyclopenta[c]pyrrole-3-carboxylate

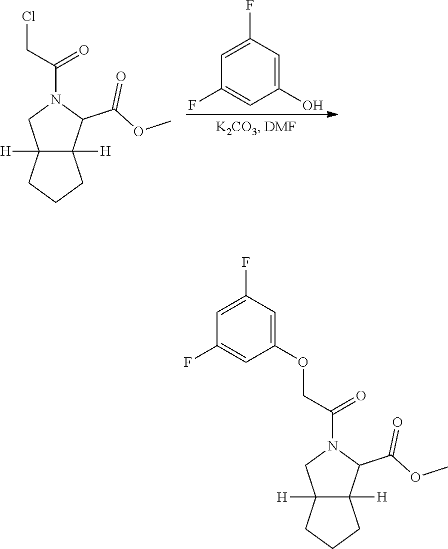

To a solution of trans-methyl 2-(2-chloroacetyl)-3,3a,4,5,6,6a-hexahydro-1H-cyclopenta[c]pyrrole-3-carboxylate (272 mg, 1.11 mmol) in DMF (10 mL) was added K₂CO₃ (306 mg, 2.21 mmol) and 3,5-difluorophenol (144 mg, 1.11 mmol). The mixture was stirred at 50° C. for 2 h. The mixture was diluted with water (50 mL) and extracted with EtOAc (30 mL×3). The organic layers were washed with brine (80 mL), dried over Na₂SO₄, filtered and concentrated in vacuum. The residue was purified by silica gel column eluted with PE to PE/EtOAc=2/1 to afford trans-methyl 2-[2-(3,5-difluorophenoxy)acetyl]-3,3a,4,5,6,6a-hexahydro-1H-cyclopenta[c]pyrrole-3-carboxylate (200 mg) as yellow oil.

MS obsd. (ESI⁺) [(M+H)⁺]: 340.2.

### Step 3: Preparation of trans-2-[2-(3,5-difluorophenoxy)acetyl]-3,3a,4,5,6,6a-hexahydro-1H-cyclopenta[c]pyrrole-3-carboxylic acid

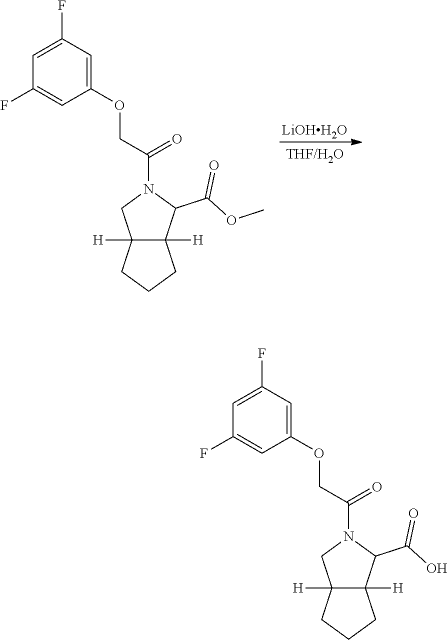 [SMILES: COC(=O)C1C2CCCC2CN1C(=O)COc1cc(F)cc(F)c1 ; "LiOH·H2O" ; O=C(O)C1C2CCCC2CN1C(=O)COc1cc(F)cc(F)c1]

To a solution of trans-methyl 2-[2-(3,5-difluorophenoxy)acetyl]-3,3a,4,5,6,6a-hexahydro-1H-cyclopenta[c]pyrrole-3-carboxylate (200 mg, 0.590 mmol) in THF (5 mL) was added a solution of LiOH·H₂O (49 mg, 1.18 mmol) in water (5 mL). The mixture was stirred at 25° C. for 1 h. The reaction mixture was diluted with water (30 mL) and washed with EtOAc (30 mL×2). The aqueous layer was acidified with concentrated HCl to pH=1˜2 and extracted with EtOAc (30 mL×3). The organic layers were dried over Na₂SO₄, filtered and concentrated in vacuum to afford trans-2-[2-(3,5-difluorophenoxy)acetyl]-3,3a,4,5,6,6a-hexahydro-1H-cyclopenta[c]pyrrole-3-carboxylic acid (190 mg) as a white solid.

MS obsd. (ESI⁺) [(M+H)⁺]: 326.1.

### Step 4: Preparation of trans-tert-butyl N-[[2-[2-(3,5-difluorophenoxy)acetyl]-3,3a,4,5,6,6a-hexahydro-1H-cyclopenta[c]pyrrole-3-carbonyl]amino]-N-[[(3S)-2-oxopyrrolidin-3-yl]methyl]carbamate

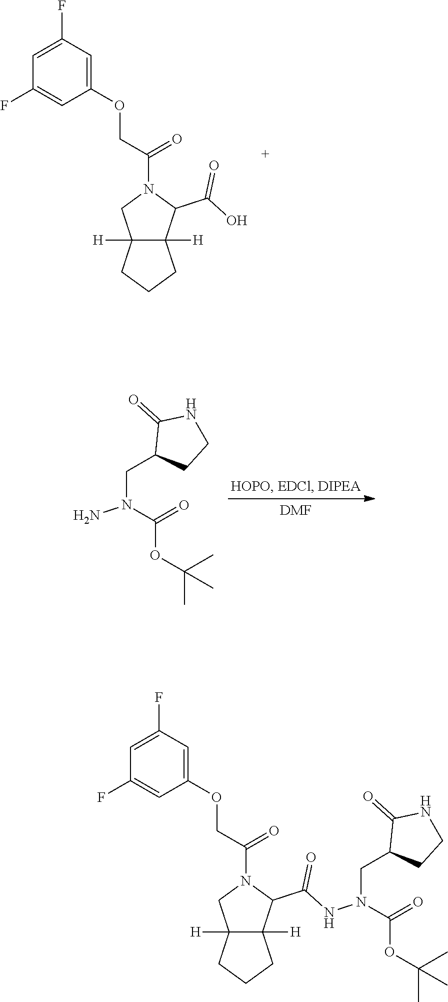

To a solution of trans-2-[2-(3,5-difluorophenoxy)acetyl]-3,3a,4,5,6,6a-hexahydro-1H-cyclopenta[c]pyrrole-3-carboxylic acid (190 mg, 0.600 mmol) in DMF (8 mL) was added DIPEA (377 mg, 2.92 mmol), EDCI (157 mg, 0.820 mmol) and HOPO (91 mg, 0.820 mmol) successively at 0° C. Then tert-butyl N-amino-N-[[(3S)-2-oxopyrrolidin-3-yl]methyl]carbamate (161 mg, 0.700 mmol, Intermediate 9) was added. The mixture was warmed to 25° C. and stirred for 13 h. The mixture was purified by reverse flash chromatography (40 g Flash Column Welch Ultimate XB_C18 20-40 μm; 120 A, water (0.1% TFA)-ACN, 45%, 40 mL/min) to afford trans-tert-butyl N-[[2-[2-(3,5-difluorophenoxy)acetyl]-3,3a,4,5,6,6a-hexahydro-1H-cyclopenta[c]pyrrole-3-carbonyl]amino]-N-[[(3S)-2-oxopyrrolidin-3-yl]methyl]carbamate (252 mg) as a white solid.

MS obsd. (ESI⁺) [(M+H)⁺]: 537.1.

### Step 5: Preparation of trans-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide

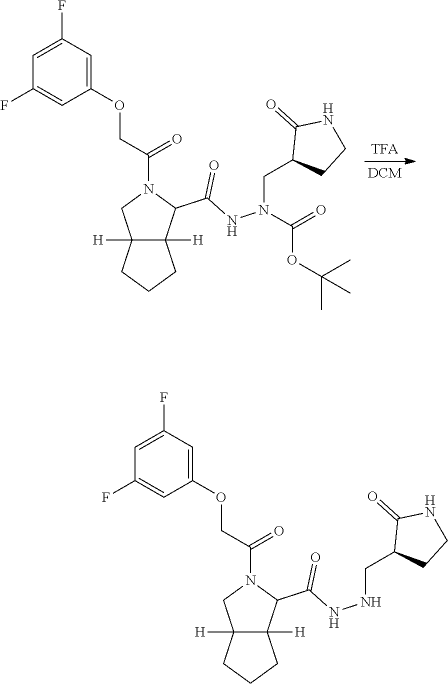 [SMILES: CC(C)(C)OC(=O)N(C[C@@H]1CCNC1=O)NC(=O)C1C2CCCC2CN1C(=O)COc1cc(F)cc(F)c1 ; O=C(NNC[C@@H]1CCNC1=O)C1C2CCCC2CN1C(=O)COc1cc(F)cc(F)c1]

To a solution of trans-tert-butyl N-[[2-[2-(3,5-difluorophenoxy)acetyl]-3,3a,4,5,6,6a-hexahydro-1H-cyclopenta[c]pyrrole-3-carbonyl]amino]-N-[[(3S)-2-oxopyrrolidin-3-yl]methyl]carbamate (252 mg, 0.470 mmol) in DCM (5 mL) was added TFA (5.0 mL). The mixture was stirred at 25° C. for 1 h. The reaction mixture was concentrated and the residue was purified by reverse flash chromatography (40 g Flash Column Welch Ultimate XB_C18 20-40 μm; 120 A, water (0.1% HCl)-ACN, 31%, 40 mL/min) to afford trans-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide (157 mg) as a white solid.

MS obsd. (ESI⁺) [(M+H)⁺]: 437.2.

### Step 6: Preparation of (3S,3aS,6aR)—N′-[(2R)-2-chloro-2-fluoro-acetyl]-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide and (3R,3aR,6aS)—N′-[(2R)-2-chloro-2-fluoro-acetyl]-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide (Example 89a and Example 89b)

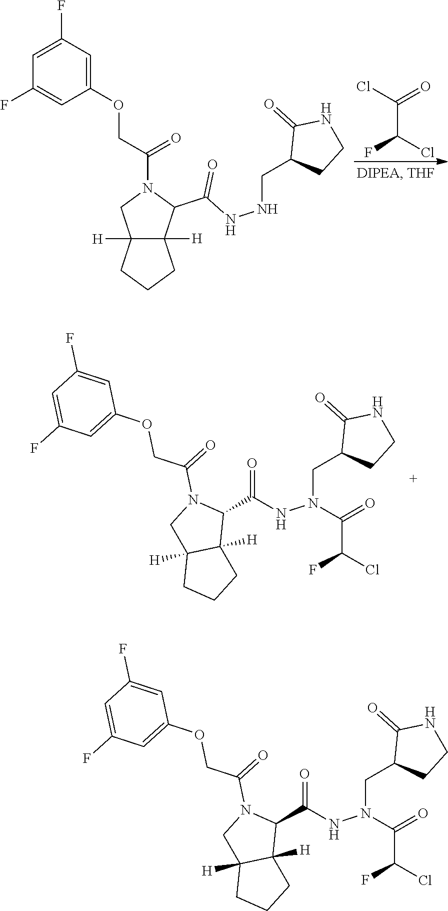

To a solution of trans-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide (170 mg, 0.360 mmol) in THF (35 mL) was added DIPEA (558 mg, 4.32 mmol) at 0° C. Then a solution of (2R)-2-chloro-2-fluoro-acetyl chloride (235 mg, 0.720 mmol in THF (5 mL) was added. The reaction mixture was stirred at 0° C. for 30 min. The reaction was quenched with MeOH (10 mL) and acidified by 4 N HCl/dioxane to pH=5 at −10° C. The resulting mixture was concentrated in vacuum at 30° C. The residue was purified by reverse flash chromatography (40 g Flash Column Welch Ultimate XB_C18 20-40 μm; 120 A, water (0.1% FA)-ACN, 45%, 40 mL/min) to afford 180 mg of a mixture of two isomers. The mixture was split by prep-SFC (Column: DAICEL CHIRALPAK IG (250 mm*30 mm, 10 um), Mobile Phase: 60% of Neu-MeOH in Supercritical CO₂, Flow Rate: 70 g/min, Cycle Time: 4.2 min, Back Pressure: 100 bar to keep the CO₂ in Supercritical flow, UV: 220 nm) to afford Example 89a (50 mg, retention time=0.895 min) and Example 89b (49 mg, retention time=1.722 min) as a white solid.

### Example 89a

¹H NMR (400 MHz, DMSO-d₆) δ: ppm 10.77-10.66 (m, 1H), 7.79-7.72 (m, 1H), 6.98-6.54 (m, 4H), 4.95-4.91 (m, 1H), 4.80 (t, J=13.2 Hz, 1H), 4.09 (s, 1H), 4.09-3.73 (m, 2H), 3.47-3.40 (m, 1H), 3.38-3.30 (m, 1H), 3.20-3.01 (m, 2H), 2.89-2.81 (m, 1H), 2.21-2.16 (m, 1H), 1.93-1.48 (m, 9H).

MS obsd. (ESI⁺) [(M+H)⁺]: 531.2.

### Example 89b

¹H NMR (400 MHz, DMSO-d₆) δ: ppm 11.01 (s, 1H), 7.76 (s, 1H), 6.78-6.66 (m, 4H), 4.86 (q, J=34.4 Hz, 15.6 Hz, 2H), 4.04-4.03 (m, 1H), 3.82-3.73 (m, 2H), 3.45 (dd, J=10.4 Hz, 3.6 Hz, 1H), 3.40-3.38 (m, 1H), 3.16-3.13 (m, 2H), 2.82-2.79 (m, 1H), 2.19-2.12 (m, 1H), 1.86-1.45 (m, 9H).

MS obsd. (ESI⁺) [(M+H)⁺]: 531.2.

### Example 90

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide

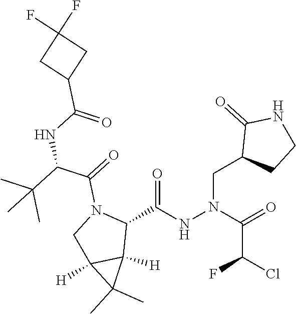 [SMILES: CC(C)(C)[C@H](NC(=O)C1CC(F)(F)C1)C(=O)N1C[C@H]2[C@@H]([C@H]1C(=O)NN(C[C@@H]1CCNC1=O)C(=O)[C@H](F)Cl)C2(C)C]

### Step 1: Preparation of methyl (1R,2S,5S)-3-[(2S)-2-(benzyloxycarbonylamino)-3,3-dimethyl-butanoyl]-6,6-dimethyl-3-azabicyclo[3.1.0]hexane-2-carboxylate

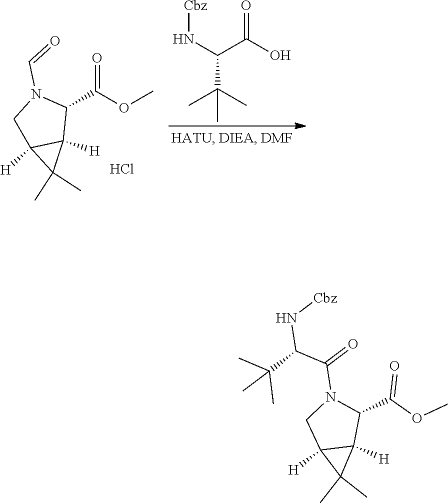

To a solution of methyl (1R,2S,5S)-6,6-dimethyl-3-azabicyclo[3.1.0]hexane-2-carboxylate; hydrochloride (2.87 g, 13.95 mmol) in DMF (10 mL) and ACN (60 mL) was added HATU (6.36 g, 16.74 mmol) and (S)-2-(((benzyloxy)carbonyl)amino)-3,3-dimethylbutanoic acid (3.7 g, 13.95 mmol). After the mixture cooling to 0° C., DIPEA (5.4 g, 41.84 mmol) was added. The resulting mixture was stirred at 25° C. for 16 h. The reaction mixture was concentrated. The residue was dissolved in EtOAc (80 mL) and washed with 1 N HCl (40 mL), brine (40 mL) and dried over Na₂SO₄. After filtration and concentration, methyl (1R,2S,5S)-3-[(2S)-2-(benzyloxycarbonylamino)-3,3-dimethyl-butanoyl]-6,6-dimethyl-3-azabicyclo[3.1.0]hexane-2-carboxylate (6.2 g, crude) was obtained as yellow oil.

### Step 2: Preparation of (1R,2S,5S)-3-[(2S)-2-(benzyloxycarbonylamino)-3,3-dimethyl-butanoyl]-6,6-dimethyl-3-azabicyclo[3.1.0]hexane-2-carboxylic acid

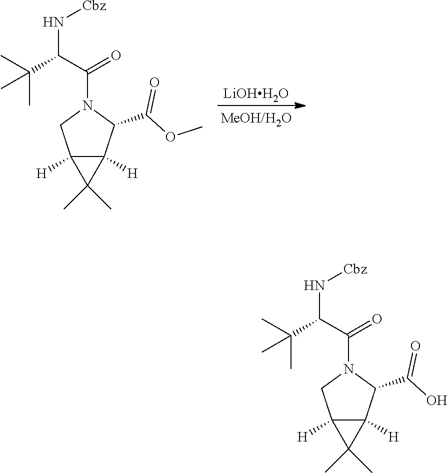

To a solution of methyl (1R,2S,5S)-3-[(2S)-2-(benzyloxycarbonylamino)-3,3-dimethyl-butanoyl]-6,6-dimethyl-3-azabicyclo[3.1.0]hexane-2-carboxylate (5.57 g, 13.38 mmol) in THF (30 mL) and H₂O (15 mL) was added LiOH·H₂O (1122 mg, 26.76 mmol). The mixture was stirred at 25° C. for 2 h. The resulting mixture was diluted with water (80 mL) and extracted with EtOAc (50 mL×2). The aqueous phase was acidified with 1 N HCl (45 mL) to pH=4˜5. The mixture was extracted with EtOAc (80 mL×3). The combined organic phase was washed with brine (80 mL), dried over Na₂SO₄ and concentrated to afford (1R,2S,5S)-3-[(2S)-2-(benzyloxycarbonylamino)-3,3-dimethyl-butanoyl]-6,6-dimethyl-3-azabicyclo[3.1.0]hexane-2-carboxylic acid (5.4 g) as colorless oil.

MS obsd. (ESI⁺) [(M+H)⁺]: 403.2.

### Step 3: Preparation of tert-butyl N-[[(1R,2S,5S)-3-[(2S)-2-(benzyloxycarbonylamino)-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate

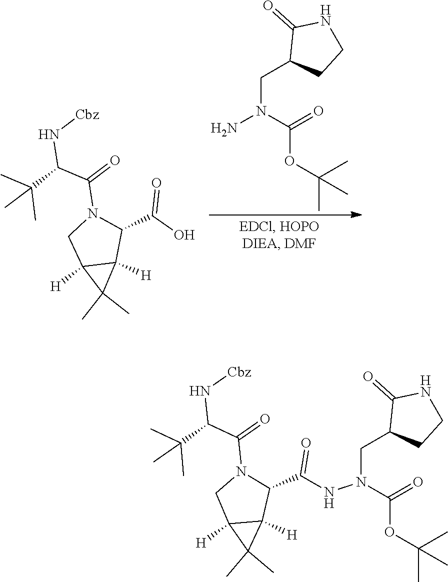 [SMILES: CC(C)(C)[C@H](NC(=O)OCc1ccccc1)C(=O)N1C[C@H]2[C@@H]([C@H]1C(=O)O)C2(C)C ; CC(C)(C)OC(=O)N(N)C[C@@H]1CCNC1=O ; CC(C)(C)OC(=O)N(C[C@@H]1CCNC1=O)NC(=O)[C@@H]1[C@@H]2[C@H](CN1C(=O)[C@@H](NC(=O)OCc1ccccc1)C(C)(C)C)C2(C)C]

To a solution of (3S,3aS,6aR)-2-[(2S)-2-(benzyloxycarbonylamino)pentanoyl]-3,3a,4,5,6,6a-hexahydro-1H-cyclopenta[c]pyrrole-3-carboxylic acid (5.38 g, 13.86 mmol) in DMF (8 mL) was added DIPEA (7.2 g, 55.44 mmol). After cooling to 0° C., HOPO (1.85 g, 16.63 mmol), EDCI (3.2 g, 16.63 mmol) and tert-butyl N-amino-N-[[(3S)-2-oxopyrrolidin-3-yl]methyl]carbamate (3.18, 13.86 mmol) was added. Then the mixture was stirred at 30° C. for 12 h. The mixture was diluted with 0.5 N HCl (100 mL) and extracted with EtOAc (80 mL×3). The organic phase was washed with 5% aqueous solution of K₂CO₃ (40 mL), brine (60 mL) and dried over Na₂SO₄. After filtration and concentration, tert-butyl N-[[(1R,2S,5S)-3-[(2S)-2-(benzyloxycarbonylamino)-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (6.87 g) was obtained as a white solid.

MS obsd. (ESI⁺) [(M+H)⁺]: 614.2.

### Step 4: Preparation of tert-butyl N-[[(1R,2S,5S)-3-[(2S)-2-amino-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate

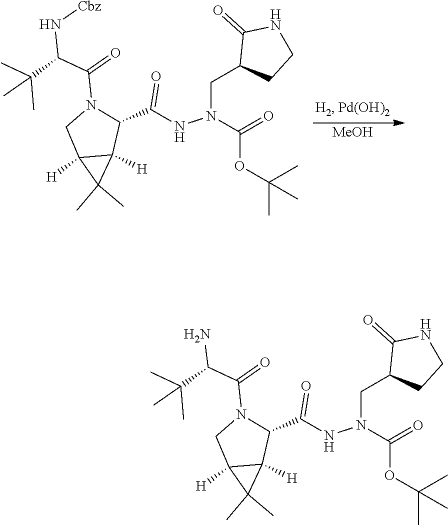

To a solution of tert-butyl N-[[(1R,2S,5S)-3-[(2S)-2-(benzyloxycarbonylamino)-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (2.5 g, 4.07 mmol) in methanol (70 mL) was added Pd(OH)₂ (250 mg). The suspension was degassed under vacuum and purged hydrogen for three times. The resulting mixture was stirred at 25° C. for 12 h under a H₂ balloon. The mixture was filtered through a pad of celite and the filtrate was concentrated to afford tert-butyl N-[[(1R,2S,5S)-3-[(2S)-2-amino-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (1.9 g) as yellow oil.

MS obsd. (ESI⁺) [(M+H)⁺]: 480.4.

### Step 5: Preparation of tert-butyl N-[[(1R,2S,5S)-3-[(2S)-2-[(3,3-difluorocyclobutanecarbonyl)amino]-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate

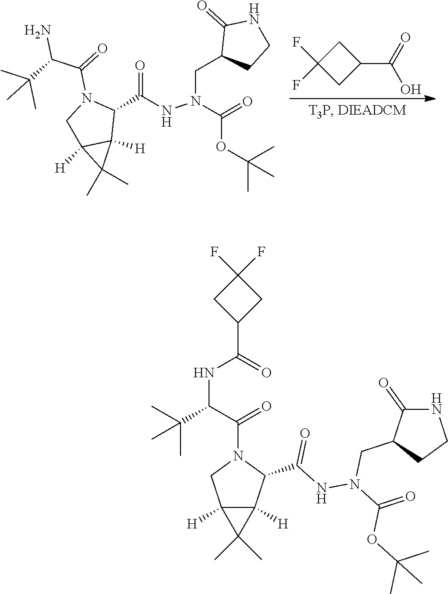

To a solution of tert-butyl N-[[(1R,2S,5S)-3-[(2S)-2-amino-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (800 mg, 1.67 mmol) in DCM (10 mL) was added DIPEA (1.08 g, 8.36 mmol), T₃P (1.6 g, 2.51 mmol, 50% in EtOAc) and 3,3-difluorocyclobutanecarboxylic acid (228 mg, 1.68 mmol) at 0° C. The reaction mixture was stirred at 25° C. for 12 h. The reaction mixture was poured into EtOAc (150 mL) and washed with 1 N HCl (50 mL×2), 5% aqueous solution of K₂CO₃ (50 mL×2) and brine (50 mL). The organic phase was dried over anhydrous Na₂SO₄, filtered and concentrated to afford tert-butyl N-[[(1R,2S,5S)-3-[(2S)-2-[(3,3-difluorocyclobutanecarbonyl)amino]-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (990 mg, 1.66 mmol) as a yellow solid.

MS obsd. (ESI⁺) [(M+H)⁺]: 598.4.

### Step 6: Preparation of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]-3,3-difluoro-cyclobutanecarboxamide; hydrochloride

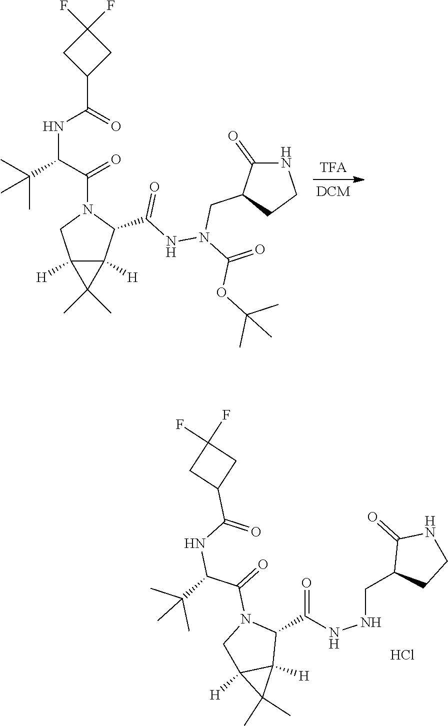 [SMILES: CC(C)(C)OC(=O)N(C[C@@H]1CCNC1=O)NC(=O)[C@@H]1[C@@H]2[C@H](CN1C(=O)[C@@H](NC(=O)C1CC(F)(F)C1)C(C)(C)C)C2(C)C ; CC(C)(C)[C@H](NC(=O)C1CC(F)(F)C1)C(=O)N1C[C@H]2[C@@H]([C@H]1C(=O)NNC[C@@H]1CCNC1=O)C2(C)C ; Cl]

To a solution of tert-butyl N-[[(1R,2S,5S)-3-[(2S)-2-[(3,3-difluorocyclobutanecarbonyl)amino]-3,3-dimethyl-butanoyl]-6,6-dimethyl-3-azabicyclo[3.1.0]hexane-2-carbonyl]amino]-N-[[(3S)-2-oxopyrrolidin-3-yl]methyl]carbamate (800 mg, 1.34 mmol) in DCM (10 mL) was added TFA (10.0 mL). The reaction mixture was stirred at 15° C. for 30 min. The reaction mixture was concentrated in vacuum to give a residue. The residue was purified by reverse flash chromatography (condition: 120 g Flash Column Welch Ultimate XB_C18 20-40 μm; 120 A, water(0.1% HCl)-ACN, 45%, 70 mL/min) to afford N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]-3,3-difluoro-cyclobutanecarboxamide; hydrochloride (690.0 mg) as a white solid.

MS obsd. (ESI⁺) [(M+H)⁺]: 498.3.

### Step 7: Preparation of N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide

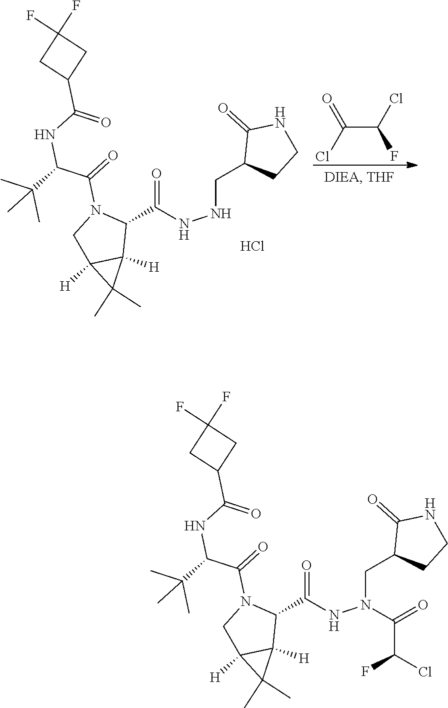

To a solution of N-[(1S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2,2-dimethyl-propyl]-3,3-difluoro-cyclobutanecarboxamide; hydrochloride (500 mg, 0.94 mmol) in THF (150 mL) was added DIPEA (1211.0 mg, 9.37 mmol). After cooling to −5° C., (2R)-2-chloro-2-fluoro-acetyl chloride (522 mg, 1.59 mmol, 40% purity, Intermediate 10) in THF (10 mL) was added at −5° C. Then the reaction mixture was stirred at the same temperature for 30 min. The reaction mixture was quenched with MeOH (10 mL) and acidified by 4 N HCl/dioxane to pH=5 at −10° C. The resulting mixture was concentrated at 30° C. The residue was purified by reversed flash chromatography (condition: 120 g Flash Column Welch Ultimate XB_C18 20-40 μm; 120 A, water (0.1% FA)-ACN, 0˜53%, 70 mL/min) to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide (Example 90, 505 mg) as a white solid.

¹H NMR (400 MHz, DMSO-d₆) δ: ppm 11.14 (s, 1H), 8.18-8.11 (m, 1H), 7.75-7.69 (m, 1H), 6.89 (d, J=50.4 Hz, 1H), 4.37 (d, J=8.4 Hz, 1H), 4.12 (s, 1H), 3.96-3.86 (m, 1H), 3.85-3.74 (m, 2H), 3.45 (dd, J=13.6 Hz, 4.4 Hz, 1H), 3.22-3.04 (m, 3H), 2.74-2.63 (m, 3H), 2.61-2.54 (m, 2H), 2.21-2.04 (m, 1H), 1.84-1.70 (m, 1H), 1.66-1.58 (m, 1H), 1.53-1.47 (m, 1H), 1.05 (s, 3H), 0.93 (s, 9H), 0.90-0.84 (m, 3H).

MS obsd. (ESI⁺) [(M+H)⁺]: 592.3.

### Example 91

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclobutanecarboxamide

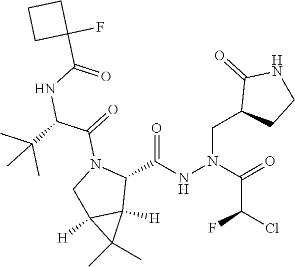 [SMILES: CC(C)(C)[C@H](NC(=O)C1(F)CCC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using 1-fluorocyclobutanecarboxylic acid instead of 3,3-difluorocyclobutanecarboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclobutanecarboxamide (Example 91) as a white solid.

¹H NMR (400 MHz, DMSO-d₆) δ: ppm 11.16 (s, 1H), 7.75-7.69 (m, 1H), 7.33-7.21 (m, 1H), 6.83 (d, J=50.4 Hz, 1H), 4.42 (d, J=8.4 Hz, 1H), 4.15 (s, 1H), 3.96-3.91 (m, 1H), 3.82-3.75 (m, 2H), 3.45 (dd, J=13.6 Hz, 4.0 Hz, 1H), 3.21-3.10 (m, 2H), 2.58-2.55 (m, 1H), 2.43-2.35 (m, 4H), 2.18-2.10 (m, 1H), 1.90-1.82 (m, 1H), 1.81-1.67 (m, 2H), 1.67-1.60 (m, 1H), 1.53-1.52 (m, 1H), 1.04 (s, 3H), 0.94-0.86 (m, 12H).

MS obsd. (ESI⁺) [(M+H)⁺]: 574.3.

### Example 92

### (1S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide

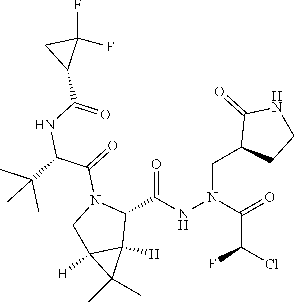 [SMILES: CC(C)(C)[C@H](NC(=O)[C@@H]1CC1(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using (1S)-2,2-difluorocyclopropanecarboxylic acid instead of 3,3-difluorocyclobutanecarboxylic acid to afford (1S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide (Example 92) as a white solid.

¹H NMR (400 MHz, DMSO-d₆) δ: ppm 11.14 (s, 1H), 8.43 (d, J=8.4 Hz, 1H), 7.75 (s, 1H), 6.90 (d, J=50.4 Hz, 1H), 4.37 (d, J=8.8 Hz, 1H), 4.13 (s, 1H), 3.95-3.88 (m, 1H), 3.82-3.73 (m, 2H), 3.44 (dd, J=14.0 Hz, 4.6 Hz, 1H), 3.20-3.12 (m, 2H), 2.90-2.80 (m, 1H), 2.65-2.56 (m, 1H), 2.18-2.11 (m, 1H), 1.86-1.73 (m, 3H), 1.65-1.59 (m, 1H), 1.51 (d, J=7.6 Hz, 1H), 1.04 (s, 3H), 0.95 (s, 9H), 0.90 (s, 3H).

MS obsd. (ESI⁺) [(M+H)⁺]: 578.1.

### Example 93

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide

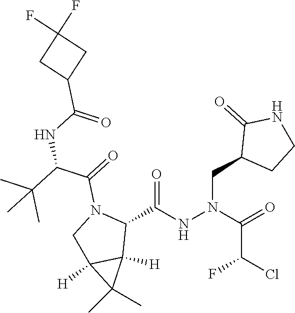 [SMILES: CC(C)(C)[C@H](NC(=O)C1CC(F)(F)C1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (2S)-2-chloro-2-fluoro-acetyl chloride (Intermediate 28) instead of (2R)-2-chloro-2-fluoro-acetyl chloride (Intermediate 10) to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide (Example 93) as a white solid.

¹H NMR (400 MHz, DMSO-d₆) δ: ppm 11.08-10.84 (m, 1H), 8.18-8.12 (m, 1H), 7.76-7.70 (m, 1H), 7.12-6.56 (m, 1H), 4.37 (t, J=8.0 Hz, 1H), 4.18-4.10 (m, 1H), 3.92-3.81 (m, 2H), 3.78-3.69 (m, 1H), 3.55-3.47 (m, 1H), 3.17-2.99 (m, 3H), 2.70-2.59 (m, 3H), 2.19-2.10 (m, 1H), 1.83-1.72 (m, 1H), 1.64-1.48 (m, 2H), 1.33-1.14 (m, 2H), 1.05-1.03 (m, 3H), 0.95-0.92 (m, 9H), 0.88 (s, 3H).

MS obsd. (ESI⁺) [(M+H)⁺]: 592.3.

### Example 94

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide

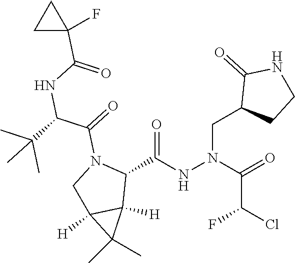 [SMILES: CC(C)(C)[C@H](NC(=O)C1(F)CC1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using 1-fluorocyclopropanecarboxylic acid instead of 3,3-difluorocyclobutanecarboxylic acid and (2S)-2-chloro-2-fluoro-acetyl chloride (Intermediate 28) instead of (2R)-2-chloro-2-fluoro-acetyl chloride (Intermediate 10) to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide (Example 94) as a white solid.

¹H NMR (400 MHz, DMSO-d₆) δ: ppm 11.08-10.87 (m, 1H), 7.75-7.70 (m, 1H), 7.40 (d, J=8.8 Hz, 1H), 7.08-6.57 (m, 1H), 4.43 (d, J=7.6 Hz, 1H), 4.23-4.15 (m, 1H), 3.92-3.88 (m, 1H), 3.79-3.65 (m, 2H), 3.55-3.51 (m, 1H), 3.16-3.00 (m, 2H), 2.66-2.58 (m, 1H), 2.21-2.15 (m, 1H), 1.83-1.74 (m, 1H), 1.64-1.48 (m, 2H), 1.35 (d, J=18.0 Hz, 2H), 1.25-1.09 (m, 2H), 1.04 (s, 3H), 0.99-0.97 (m, 9H), 0.87 (s, 3H).

MS obsd. (ESI⁺) [(M+H)⁺]: 560.2.

### Example 95

### (1S)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide

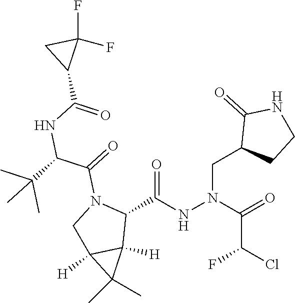 [SMILES: CC(C)(C)[C@H](NC(=O)[C@@H]1CC1(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (1S)-2,2-difluorocyclopropanecarboxylic acid instead of 3,3-difluorocyclobutanecarboxylic acid and (2S)-2-chloro-2-fluoro-acetyl chloride (Intermediate 28) instead of (2R)-2-chloro-2-fluoro-acetyl chloride (Intermediate 10) to afford (1S)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide (Example 95) as a white solid.

¹H NMR (400 MHz, DMSO-d₆) δ: ppm 11.01-10.83 (m, 1H), 8.42 (d, J=8.8 Hz, 1H), 7.76-7.70 (m, 1H), 7.13-6.56 (m, 1H), 4.38 (d, J=8.8 Hz, 1H), 4.19-4.12 (m, 1H), 3.89-3.84 (m, 1H), 3.77-3.69 (m, 2H), 3.57-3.46 (m, 1H), 3.26-3.16 (m, 1H), 3.14-3.00 (m, 1H), 2.90-2.82 (m, 1H), 2.72-2.63 (m, 1H), 2.21-2.07 (m, 1H), 1.87-1.73 (m, 3H), 1.66-1.48 (m, 2H), 1.09-1.01 (m, 3H), 1.01-0.90 (m, 9H), 0.90-0.84 (m, 3H).

MS obsd. (ESI⁺) [(M+H)⁺]: 578.2.

### Example 96

### (1R)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide

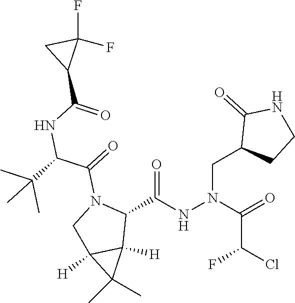 [SMILES: CC(C)(C)[C@H](NC(=O)[C@H]1CC1(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (1R)-2,2-difluorocyclopropanecarboxylic acid instead of 3,3-difluorocyclobutanecarboxylic acid and (2S)-2-chloro-2-fluoro-acetyl chloride (Intermediate 28) instead of (2R)-2-chloro-2-fluoro-acetyl chloride (Intermediate 10) to afford (1R)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide (Example 96) as a white solid.

¹H NMR (400 MHz, DMSO-d₆) δ: ppm 11.10-10.86 (m, 1H), 8.48 (d, J=8.8 Hz, 1H), 7.76-7.70 (m, 1H), 7.15-6.57 (m, 1H), 4.43-4.41 (m, 1H), 4.21-4.12 (m, 1H), 3.91-3.69 (m, 3H), 3.53 (dd, J=14.0 Hz, 4.0 Hz, 1H), 3.25-3.13 (m, 1H), 3.05-3.01 (m, 1H), 2.90-2.73 (m, 1H), 2.65-2.55 (m, 1H), 2.24-2.12 (m, 1H), 1.92-1.86 (m, 1H), 1.85-1.76 (m, 2H), 1.63-1.47 (m, 2H), 1.04 (s, 3H), 0.97-0.95 (m, 9H), 0.83 (s, 3H).

MS obsd. (ESI⁺) [(M+H)⁺]: 578.1.

### Example 97

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclobutanecarboxamide

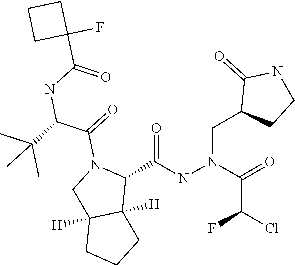 [SMILES: CC(C)(C)[C@H](NC(=O)C1(F)CCC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using 1-fluorocyclobutanecarboxylic acid and T₃P instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclobutanecarboxamide (Example 97) as a white solid.

¹H NMR (400 MHz, CDCl₃) δ: ppm 9.95 (br. s, 1H), 6.91 (br. s, 1H), 6.82 (d, J=51.6 Hz, 1H), 6.05 (br. s, 1H), 4.67 (d, J=9.2 Hz, 1H), 4.50-4.41 (m, 1H), 4.22-4.12 (m, 1H), 4.00 (dd, J=10.4 Hz, 7.2 Hz, 1H), 3.78 (dd, J=10.4 Hz, 2.8 Hz, 1H), 3.44-3.35 (m, 2H), 2.85-2.56 (m, 5H), 2.49-2.33 (m, 3H), 2.00-1.79 (m, 5H), 1.76-1.59 (m, 4H), 1.50-1.42 (m, 1H), 1.02 (s, 9H).

MS obsd. (ESI⁺) [(M+H)⁺]: 574.2.

### Example 98

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-3,3-difluoro-cyclobutanecarboxamide

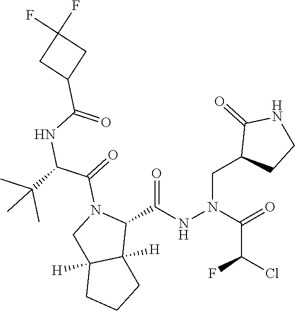 [SMILES: CC(C)(C)[C@H](NC(=O)C1CC(F)(F)C1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using 3,3-difluorocyclobutanecarboxylic acid and T₃P instead of acetic anhydride to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-3,3-difluoro-cyclobutanecarboxamide (Example 98) as a white solid.

¹H NMR (400 MHz, DMSO-d₆) δ: ppm 11.07 (s, 1H), 8.17 (d, J=8.8 Hz, 1H), 7.77 (s, 1H), 7.01 (d, J=50.8 Hz, 1H), 4.45 (d, J=8.4 Hz, 1H), 3.94 (d, J=6.4 Hz, 1H), 3.91-3.84 (m, 1H), 3.78-3.68 (m, 2H), 3.39 (dd, J=13.6 Hz, 4.0 Hz, 1H), 3.32 (s, 2H), 3.29-3.08 (m, 3H), 2.77-2.57 (m, 6H), 2.20-2.11 (m, 1H), 1.91-1.81 (m, 1H), 1.79-1.72 (m, 3H), 1.70-1.59 (m, 1H), 1.40-1.30 (m, 1H), 0.93 (s, 9H).

MS obsd. (ESI⁺) [(M+H)⁺]: 592.3.

### Example 99

### (1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

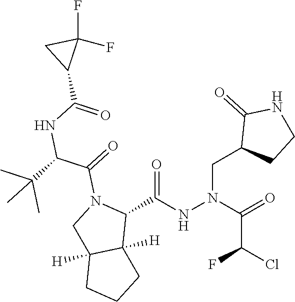 [SMILES: CC(C)(C)[C@H](NC(=O)[C@@H]1CC1(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (1S)-2,2-difluorocyclopropanecarboxylic acid and T₃P instead of acetic anhydride to afford (1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (Example 99) as a white solid.

¹H NMR (400 MHz, DMSO-d₆) δ: ppm 11.18 (s, 1H), 8.48 (d, J=8.8 Hz, 1H), 7.77 (s, 1H), 7.00 (d, J=50.6 Hz, 1H), 4.47 (d, J=8.8 Hz, 1H), 4.04-3.93 (m, 1H), 3.90-3.83 (m, 1H), 3.80-3.60 (m, 2H), 3.90 (dd, J=13.6 Hz, 4.0 Hz, 1H), 3.22-3.10 (m, 2H), 2.94-2.84 (m, 1H), 2.75-2.70 (m, 1H), 2.54-2.52 (m, 2H), 2.20-2.10 (m, 1H), 1.91-1.81 (m, 3H), 1.81-1.70 (m, 4H), 1.68-1.57 (m, 1H), 1.39-1.30 (m, 1H), 0.92 (s, 9H).

MS obsd. (ESI⁺) [(M+H)⁺]: 578.3.

### Example 100

### (1R)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

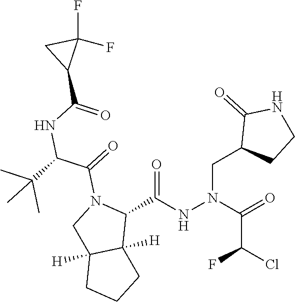 [SMILES: CC(C)(C)[C@H](NC(=O)[C@H]1CC1(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 29, by using (1R)-2,2-difluorocyclopropanecarboxylic acid and T₃P instead of acetic anhydride to afford (1R)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (Example 100) as a white solid.

¹H NMR (400 MHz, DMSO-d₆) δ: ppm 11.11 (s, 1H), 8.51 (d, J=8.8 Hz, 1H), 7.77-7.70 (m, 1H), 7.02 (d, J=50.8 Hz, 1H), 4.48 (d, J=8.8 Hz, 1H), 3.95 (d, J=6.4 Hz, 1H), 3.88-3.83 (m, 1H), 3.73-3.70 (m, 2H), 3.41-3.36 (dd, J=14.0 Hz, 4.0 Hz, 1H), 3.21-3.10 (m, 2H), 2.90-2.82 (m, 1H), 2.76-2.66 (m, 1H), 2.59-2.54 (m, 1H), 2.20-2.11 (m, 1H), 1.94-1.79 (m, 4H), 1.79-1.71 (m, 3H), 1.71-1.58 (m, 2H), 1.35-1.26 (m, 1H), 0.95 (s, 9H).

MS obsd. (ESI⁺) [(M+H)⁺]: 578.4.

### Example 101

### (1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

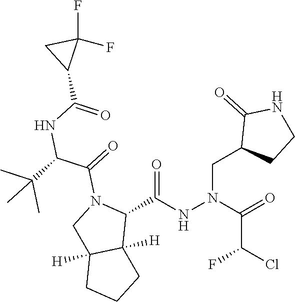 [SMILES: CC(C)(C)[C@H](NC(=O)[C@@H]1CC1(F)F)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 29, by using (1S)-2,2-difluorocyclopropanecarboxylic acid and T₃P instead of acetic anhydride and (2S)-2-chloro-2-fluoro-acetyl chloride (Intermediate 28) instead of (2R)-2-chloro-2-fluoro-acetyl chloride (Intermediate 10) to afford (1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (Example 101) as a white solid.

¹H NMR (400 MHz, DMSO-d₆) δ: ppm 11.05-10.73 (m, 1H), 8.51-8.43 (m, 1H), 7.77-7.70 (m, 1H), 7.21-6.39 (m, 1H), 4.48 (d, J=8.8 Hz, 1H), 4.02-3.92 (m, 1H), 3.89-3.79 (m, 2H), 3.72-3.66 (m, 1H), 3.51-3.43 (m, 1H), 3.18-2.96 (m, 2H), 2.90-2.76 (m, 2H), 2.28-2.13 (m, 2H), 1.93-1.58 (m, 9H), 1.41-1.29 (m, 1H), 1.02-0.92 (m, 9H).

MS obsd. (ESI⁺) [(M+Na)⁺]: 600.2.

### Example 102

### N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide

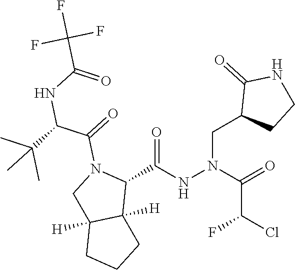 [SMILES: CC(C)(C)[C@H](NC(=O)C(F)(F)F)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 29, by using trifluoroacetic anhydride instead of acetic anhydride and (2S)-2-chloro-2-fluoro-acetyl chloride (Intermediate 28) instead of (2R)-2-chloro-2-fluoro-acetyl chloride (Intermediate 10) to afford N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide (Example 102) as a white solid.

¹H NMR (400 MHz, DMSO-d₆) δ: ppm 11.06-10.77 (m, 1H), 9.44-9.39 (m, 1H), 7.76-7.71 (m, 1H), 7.14-6.40 (m, 1H), 4.53-4.50 (m, 1H), 4.07-4.02 (m, 1H), 3.88-3.69 (m, 3H), 3.56-3.50 (m, 1H), 3.21-3.00 (m, 2H), 2.80-2.70 (m, 2H), 2.64-2.52 (m, 1H), 2.26-2.09 (m, 1H), 1.92-1.56 (m, 6H), 1.43-1.29 (m, 1H), 1.01-0.99 (m, 9H).

MS obsd. (ESI⁺) [(M+H)⁺]: 570.0.

### Example 103

### N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide

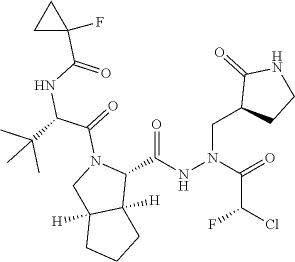 [SMILES: CC(C)(C)[C@H](NC(=O)C1(F)CC1)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 29, by using 1-fluorocyclopropanecarboxylic acid and T₃P instead of acetic anhydride and (2S)-2-chloro-2-fluoro-acetyl chloride (Intermediate 28) instead of (2R)-2-chloro-2-fluoro-acetyl chloride (Intermediate 10) to afford N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide (Example 103) as a white solid.

¹H NMR (400 MHz, DMSO-d₆) δ: ppm 11.04-10.76 (m, 1H), 7.70-7.74 (m, 1H), 7.39 (d, J=8.4 Hz, 1H), 7.16-6.42 (m, 1H), 4.52 (d, J=8.4 Hz, 1H), 4.10-4.00 (m, 1H), 3.92-3.82 (m, 1H), 3.80-3.65 (m, 2H), 3.55-3.49 (m, 1H), 3.22-3.00 (m, 2H), 2.80-2.71 (m, 2H), 2.62-2.53 (m, 1H), 2.28-2.10 (m, 1H), 1.90-1.55 (m, 6H), 1.43-1.30 (m, 3H), 1.26-1.13 (m, 2H), 1.00-0.97 (m, 9H).

MS obsd. (ESI⁺) [(M+H)⁺]: 560.3.

### Example 104

### (1R)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

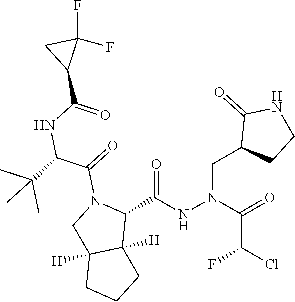 [SMILES: CC(C)(C)[C@H](NC(=O)[C@H]1CC1(F)F)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 29, by using (1R)-2,2-difluorocyclopropanecarboxylic acid and T₃P instead of acetic anhydride and (2S)-2-chloro-2-fluoro-acetyl chloride (Intermediate 28) instead of (2R)-2-chloro-2-fluoro-acetyl chloride (Intermediate 10) to afford (1R)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (Example 104) as a white solid.

¹H NMR (400 MHz, DMSO-d₆) δ: ppm 11.06-10.73 (m, 1H), 8.48 (d, J=10.0 Hz, 1H), 7.76-7.70 (m, 1H), 7.21-6.39 (m, 1H), 4.47 (d, J=8.8 Hz, 1H), 4.00-3.95 (m, 1H), 3.85-3.79 (m, 1H), 3.73-3.71 (m, 1H), 3.50-3.44 (m, 1H), 3.20-3.16 (m, 2H), 3.14-2.99 (m, 1H), 2.84-2.73 (m, 2H), 2.71-2.67 (m, 2H), 2.21-2.16 (m, 1H), 1.84-1.66 (m, 8H), 1.32-1.29 (m, 1H), 0.97-0.95 (m, 9H).

MS obsd. (ESI⁺) [(M+H)⁺]: 578.2.

### Example 105

### N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide

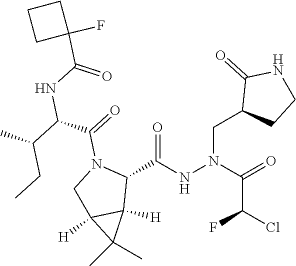 [SMILES: CC[C@H](C)[C@H](NC(=O)C1(F)CCC1)C(=O)N1C[C@H]2[C@@H]([C@H]1C(=O)NN(C[C@@H]1CCNC1=O)C(=O)[C@H](F)Cl)C2(C)C]

### Step 1: Preparation of methyl (1R,2S,5S)-3-[(2S,3S)-2-(benzyloxycarbonylamino)-3-methyl-pentanoyl]-6,6-dimethyl-3-azabicyclo[3.1.0]hexane-2-carboxylate

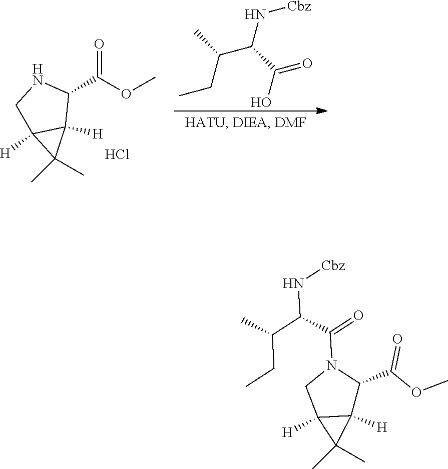

To a solution of Z-ILe-OH (2.84 g, 10.7 mmol) in DMF (30 mL) was added DIPEA (5.03 g, 38.9 mmol), HATU (4.44 g, 11.67 mmol) and methyl (1R,2S,5S)-6,6-dimethyl-3-azabicyclo[3.1.0]hexane-2-carboxylate; hydrochloride (2.0 g, 9.72 mmol) at 0° C. The reaction mixture was stirred at 25° C. for 2 h. The resulting mixture was diluted with water (50 mL) and extracted with EtOAc (150 mL×3). The combined organic layers were washed with brine (100 mL) and dried over Na₂SO₄, filtered and concentrated. The residue was purified by silica gel column eluted with PE to PE/EtOAc=2/1 to afford methyl (1R,2S,5S)-3-[(2S,3S)-2-(benzyloxycarbonylamino)-3-methyl-pentanoyl]-6,6-dimethyl-3-azabicyclo[3.1.0]hexane-2-carboxylate (3.03 g) as a yellow solid.

MS obsd. (ESI⁺) [(M+H)⁺]: 417.3.

### Step 2: Preparation of (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid

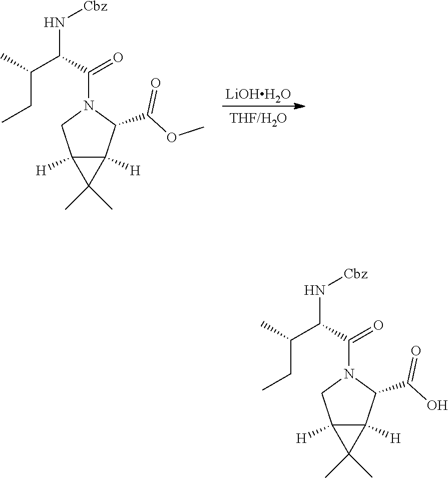

To a solution of methyl (1R,2S,5S)-3-[(2S,3S)-2-(benzyloxycarbonylamino)-3-methyl-pentanoyl]-6,6-dimethyl-3-azabicyclo[3.1.0]hexane-2-carboxylate (3.0 g, 7.2 mmol) in THF (20 mL) was added a solution of LiOH·H₂O (605 mg, 14.42 mmol) in water (20 mL) at 0° C. The reaction mixture was stirred at 25° C. for 150 min. The resulting mixture was diluted with 1 N HCl (50 mL) and extracted with EtOAc (50 mL×3). The combined organic layers were washed with brine (50 mL), dried over Na₂SO₄, filtered and concentrated in vacuum to afford (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid (2.42 g) as colorless oil.

MS obsd. (ESI⁺) [(M+H)⁺]: 403.2.

### Step 3: Preparation of tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

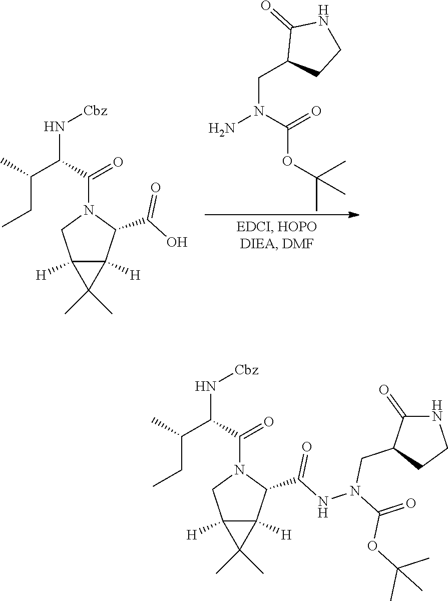 [SMILES: CC[C@H](C)[C@H](NC(=O)OCc1ccccc1)C(=O)N1C[C@H]2[C@@H]([C@H]1C(=O)O)C2(C)C ; CC(C)(C)OC(=O)N(N)C[C@@H]1CCNC1=O ; CC[C@H](C)[C@H](NC(=O)OCc1ccccc1)C(=O)N1C[C@H]2[C@@H]([C@H]1C(=O)NN(C[C@@H]1CCNC1=O)C(=O)OC(C)(C)C)C2(C)C]

To a solution of (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid (2.42 g, 6.01 mmol) in DMF (30 mL) was added DIPEA (3.89 g, 30.06 mmol), EDCI (1.38 g, 7.22 mmol) and HOPO (0.8 g, 7.22 mmol) at 0° C. Then tert-butyl N-amino-N-[[(3S)-2-oxopyrrolidin-3-yl]methyl]carbamate (1.65 g, 7.2 mmol) was added to the mixture at 0° C. The reaction mixture was stirred at 25° C. for 12 h. The mixture was diluted with brine (50 mL) and acidified with 1N HCl (10 mL). The mixture was extracted with EtOAc (50 mL×3). The combined organic phase was washed with brine (100 mL), dried over Na₂SO₄, filtered and concentrated under vacuum. The residue was purified by reverse flash chromatography eluted with ACN in H₂O (0.1% TFA)=0˜60% to tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (2.48 g) as a white solid.

MS obsd. (ESI⁺) [(M+H)⁺]: 614.4.

### Step 4: Preparation of tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

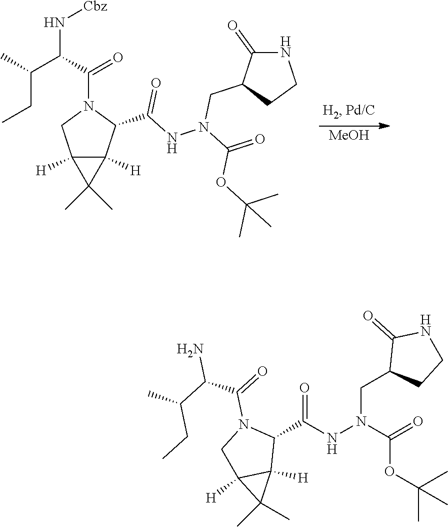 [SMILES: "MeOH" ; CC[C@H](C)[C@H](NC(=O)OCc1ccccc1)C(=O)N1C[C@H]2[C@@H]([C@H]1C(=O)NN(C[C@@H]1CCNC1=O)C(=O)OC(C)(C)C)C2(C)C ; CC[C@H](C)[C@H](N)C(=O)N1C[C@H]2[C@@H]([C@H]1C(=O)NN(C[C@@H]1CCNC1=O)C(=O)OC(C)(C)C)C2(C)C]

To a solution of tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (2.48 g, 4.04 mmol) in methanol (30 mL) was added Pd/C (0.5 g, 10% purity). The suspension was degassed under vacuum and purged H₂ for 3 times. The suspension was stirred at 25° C. for 2 h under a H₂ balloon. The resulting suspension was filtered and the filtrate was concentrated to afford tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (1.9 g) as a white solid.

MS obsd. (ESI⁺) [(M+H)⁺]: 480.3.

### Step 5: Preparation of tert-butyl N-[[(1R,2S,5S)-3-[(2S,3S)-2-[(1-fluorocyclobutanecarbonyl)amino]-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

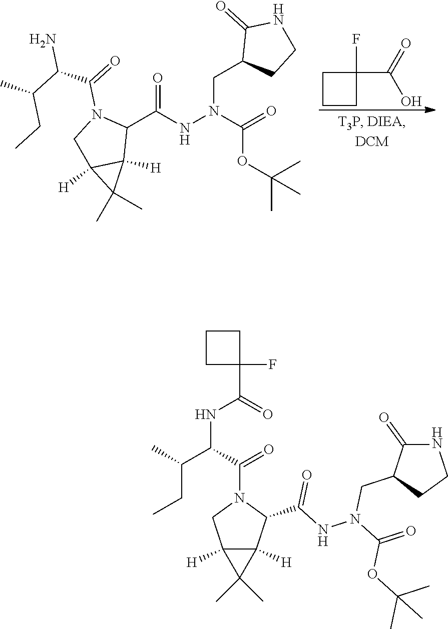

To a solution of tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (358 mg, 0.75 mmol) in DCM (15 mL) was added DIPEA (438 mg, 3.39 mmol), 1-fluorocyclobutanecarboxylic acid (80 mg, 0.68 mmol) and T₃P (647 mg, 1.02 mmol, 50% in EtOAc) at 0° C. The reaction mixture was stirred at 25° C. for 30 min. The reaction mixture was poured into water (50 mL) and extracted with DCM (50 mL×3). The combined organic phase was washed with brine (50 mL), dried over anhydrous Na₂SO₄, filtered and concentrated to afford tert-butyl N-[[(1R,2S,5S)-3-[(2S,3S)-2-[(1-fluorocyclobutanecarbonyl)amino]-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (390 mg) as a white solid.

MS obsd. (ESI⁺) [(M+H)⁺]: 580.4.

### Step 6: Preparation of N-[(1S,2S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-1-fluoro-cyclobutanecarboxamide; hydrochloride

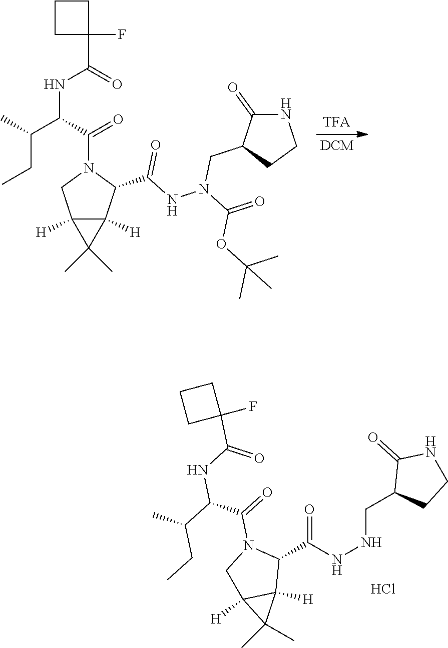 [SMILES: CC[C@H](C)[C@H](NC(=O)C1(F)CCC1)C(=O)N1C[C@H]2[C@@H]([C@H]1C(=O)NN(C[C@@H]1CCNC1=O)C(=O)OC(C)(C)C)C2(C)C ; CC[C@H](C)[C@H](NC(=O)C1(F)CCC1)C(=O)N1C[C@H]2[C@@H]([C@H]1C(=O)NNC[C@@H]1CCNC1=O)C2(C)C ; Cl]

To a solution of tert-butyl N-[[(1R,2S,5S)-3-[(2S,3S)-2-[(1-fluorocyclobutanecarbonyl)amino]-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (350 mg, 0.6 mmol) in DCM (5 mL) was added TFA (5.0 mL). The mixture was stirred at 25° C. for 30 min. The resulting solution was concentrated in vacuum and the residue was purified by reverse flash chromatography (condition: 120 g Flash Column Welch Ultimate XB_C18 20-40 μm; 120 A, water(0.1% HCl)-ACN, 46%, 70 mL/min) to afford N-[(1S,2S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-1-fluoro-cyclobutanecarboxamide; hydrochloride (300 mg) as a white solid.

MS obsd. (ESI⁺) [(M+H)⁺]: 480.4.

### Step 7: Preparation of N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide

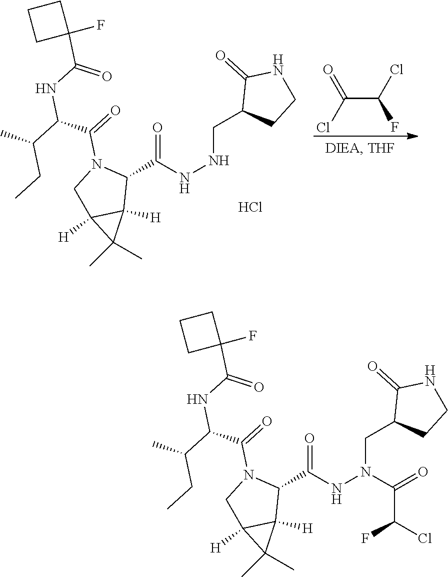

To a solution of N-[(1S,2S)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-1-fluoro-cyclobutanecarboxamide; hydrochloride (270 mg, 0.52 mmol) in THF (90 mL) was added DIPEA (677 mg, 5.24 mmol). After cooling to −5° C., (2R)-2-chloro-2-fluoro-acetyl chloride (292.0 mg, 0.89 mmol, 40% purity, Intermediate 10) in THF (10 mL) was added into the mixture at −5° C. Then the reaction mixture was stirred at −5° C. for 30 min. The reaction was quenched with MeOH (10 mL) and acidified by 4 N HCl/dioxane to pH=5 at −10° C. The resulting mixture was concentrated in vacuum at 30° C. The residue was purified by reversed flash chromatography (condition: 120 g Flash Column Welch Ultimate XB_C18 20-40 μm; 120 A, water (0.1% FA)-ACN, 0˜59%, 70 mL/min) to afford N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide (174 mg, Example 105) as a white solid.

¹H NMR (400 MHz, DMSO-d₆) δ: ppm 11.13 (s, 1H), 8.26-8.17 (m, 1H), 7.75-7.70 (m, 1H), 6.85 (d, J=50.4 Hz, 1H), 4.22 (t, J=8.0 Hz, 1H), 4.13 (s, 1H), 4.02-3.94 (m, 1H), 3.88-3.84 (m, 1H), 3.79 (dd, J=14.0 Hz, 9.6 Hz, 1H), 3.44 (dd, J=13.6 Hz, 4.0 Hz, 1H), 3.24-3.11 (m, 2H), 2.63-2.54 (m, 1H), 2.53-2.21 (m, 4H), 2.17-2.09 (m, 1H), 1.95-1.81 (m, 2H), 1.79-1.67 (m, 2H), 1.65-1.60 (m, 1H), 1.53-1.49 (d, J=7.6 Hz, 1H), 1.47-1.39 (m, 1H), 1.08-1.01 (m, 4H), 0.90 (s, 3H) 0.81-0.77 (m, 6H).

MS obsd. (ESI⁺) [(M+H)⁺]: 574.2.

### Example 106

### N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide

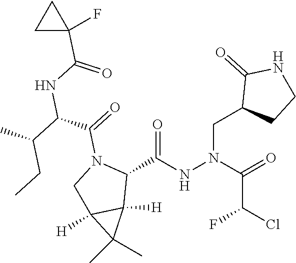 [SMILES: CC[C@H](C)[C@H](NC(=O)C1(F)CC1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 105, by using 1-fluorocyclopropanecarboxylic acid instead of 1-fluorocyclobutanecarboxylic acid, and (2S)-2-chloro-2-fluoro-acetyl chloride (Intermediate 28) instead of (2R)-2-chloro-2-fluoro-acetyl chloride (Intermediate 10) to afford N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide (Example 106) as a white solid.

¹H NMR (400 MHz, DMSO-d₆) δ: ppm 10.07-10.83 (m, 1H), 8.57-8.42 (m, 1H), 7.76-7.72 (m, 1H), 7.16-6.57 (m, 1H), 4.28-4.10 (m, 2H), 3.94 (d, J=10.4 Hz, 1H), 3.87-3.81 (m, 1H), 3.80-3.63 (m, 1H), 3.63-3.50 (m, 1H), 3.19-2.96 (m, 2H), 2.79-2.60 (m, 1H), 2.25-2.09 (m, 1H), 2.05-1.87 (m, 1H), 1.84-1.71 (m, 1H), 1.66-1.52 (m, 2H), 1.51-1.45 (m, 1H), 1.33-1.28 (m, 1H), 1.27-1.23 (m, 1H), 1.22-1.13 (m, 1H), 1.11-1.01 (m, 5H), 0.88 (s, 3H), 0.84 (d, J=6.8 Hz, 3H), 0.80 (t, J=7.2 Hz, 3H).

MS obsd. (ESI⁺) [(M+H)⁺]: 560.3.

### Example 107

### N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide

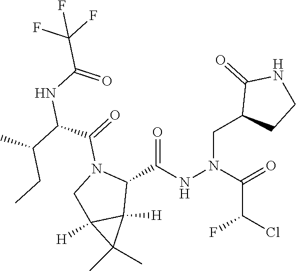 [SMILES: CC[C@H](C)[C@H](NC(=O)C(F)(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 105, by using 2,2,2-trifluoroacetic acid instead of 1-fluorocyclobutanecarboxylic acid, and (2S)-2-chloro-2-fluoro-acetyl chloride (Intermediate 28) instead of (2R)-2-chloro-2-fluoro-acetyl chloride (Intermediate 10) to afford N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide (Example 107) as a white solid. ¹H NMR (400 MHz, DMSO-d₆) δ: ppm 11.10-10.87 (m, 1H), 10.00-9.90 (m, 1H), 7.75-7.71 (m, 1H), 7.15-6.58 (m, 1H), 4.23-4.14 (m, 2H), 3.92-3.83 (m, 2H), 3.82-3.64 (m, 1H), 3.55 (dd, J=14.0 Hz, 4.4 Hz, 1H), 3.20-2.98 (m, 2H), 2.75-2.55 (m, 1H), 2.22-2.11 (m, 1H), 2.04-1.86 (m, 1H), 1.84-1.73 (m, 1H), 1.69-1.43 (m, 3H), 1.18-1.09 (m, 1H), 1.06 (d, J=7.2 Hz, 3H), 0.92-0.87 (m, 6H), 0.82 (t, J=7.2 Hz, 3H).

MS obsd. (ESI⁺) [(M+H)⁺]: 570.1.

### Example 108

### N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide

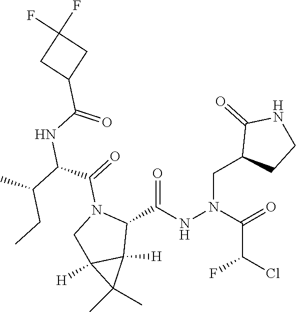 [SMILES: CC[C@H](C)[C@H](NC(=O)C1CC(F)(F)C1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 105, by using 3,3-difluorocyclobutanecarboxylic acid instead of 1-fluorocyclobutanecarboxylic acid, and (2S)-2-chloro-2-fluoro-acetyl chloride (Intermediate 28) instead of (2R)-2-chloro-2-fluoro-acetyl chloride (Intermediate 10) to afford N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide (Example 108) as a white solid.

¹H NMR (400 MHz, DMSO-d₆) δ: ppm 11.05-10.81 (m, 1H), 8.44-8.39 (m, 1H), 7.78-7.69 (m, 1H), 7.17-6.57 (m, 1H), 4.25-4.10 (m, 2H), 3.95-3.89 (m, 1H), 3.88-3.82 (m, 1H), 3.81-3.63 (m, 1H), 3.58-3.50 (m, 1H), 3.20-2.85 (m, 3H), 2.75-2.62 (m, 3H), 2.60-2.50 (m, 1H), 2.25-2.10 (m, 1H), 1.85-1.70 (m, 2H), 1.65-1.40 (m, 3H), 1.30-1.19 (m, 1H), 1.14-1.03 (m, 4H), 0.89 (s, 3H), 0.84 (d, J=6.4 Hz, 3H), 0.80 (t, J=7.6 Hz, 3H).

MS obsd. (ESI⁺) [(M+H)⁺]: 592.4.

### Example 109

### N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide

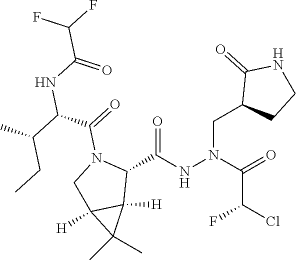 [SMILES: CC[C@H](C)[C@H](NC(=O)C(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 105, by using 2,2-difluoroacetic acid instead of 1-fluorocyclobutanecarboxylic acid, and (2S)-2-chloro-2-fluoro-acetyl chloride (Intermediate 28) instead of (2R)-2-chloro-2-fluoro-acetyl chloride (Intermediate 10) to afford N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide (Example 109) as a white solid.

¹H NMR (400 MHz, DMSO-d₆) δ: ppm 11.09-10.86 (m, 1H), 9.26-9.20 (m, 1H), 7.75-7.71 (m, 1H), 7.15-6.58 (m, 1H), 6.20 (t, J=53.6 Hz, 1H), 4.25-1.42 (m, 2H), 3.93-3.82 (m, 2H), 3.73-3.55 (m, 2H), 3.20-3.04 (m, 2H), 2.24-2.12 (m, 1H), 1.92-1.76 (m, 2H), 1.70-1.57 (m, 2H), 1.55-1.40 (m, 2H), 1.13-1.03 (m, 4H), 0.90 (s, 3H), 0.88 (d, J=6.8 Hz, 3H), 0.81 (t, J=7.2 Hz, 3H).

MS obsd. (ESI⁺) [(M+H)⁺]: 552.3.

### Example 110

### N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide

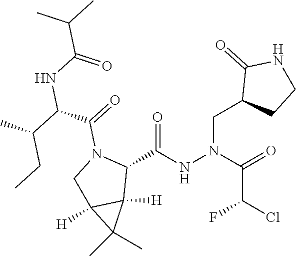 [SMILES: CC[C@H](C)[C@H](NC(=O)C(C)C)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 105, by using isobutyryl chloride without coupling reagent instead of 1-fluorocyclobutanecarboxylic acid, and (2S)-2-chloro-2-fluoro-acetyl chloride (Intermediate 28) instead of (2R)-2-chloro-2-fluoro-acetyl chloride (Intermediate 10) to afford N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide (Example 110) as a white solid.

¹H NMR (400 MHz, DMSO-d₆) δ: ppm 11.05-10.81 (m, 1H), 8.09 (t, J=8.0 Hz, 1H), 7.73 (d, J=19.2 Hz, 1H), 7.18-6.57 (m, 1H), 4.21-4.11 (m, 2H), 3.96 (d, J=10.0 Hz, 1H), 3.87-3.65 (m, 2H), 3.58-3.50 (m, 1H), 3.20-2.95 (m, 2H), 2.75-2.60 (m, 1H), 2.45-2.37 (m, 1H), 2.23-2.10 (m, 1H), 1.85-1.70 (m, 2H), 1.65-1.42 (m, 3H), 1.15-1.07 (m, 1H), 1.05 (s, 3H), 0.96 (d, J=6.4 Hz, 3H), 0.91 (d, J=6.8 Hz, 3H), 0.88 (s, 3H), 0.83-0.77 (m, 6H).

MS obsd. (ESI⁺) [(M+H)⁺]: 544.3.

### Example 111

### Rac-2,2-difluoro-N-[(1S,2S)-2-methyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide

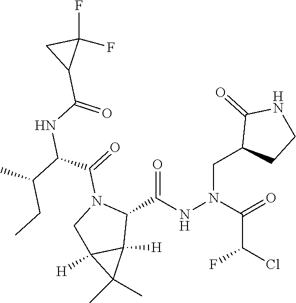 [SMILES: CC[C@H](C)[C@H](NC(=O)C1CC1(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 105, by using 2,2-difluorocyclopropanecarboxylic acid instead of 1-fluorocyclobutanecarboxylic acid, and (2S)-2-chloro-2-fluoro-acetyl chloride (Intermediate 28) instead of (2R)-2-chloro-2-fluoro-acetyl chloride (Intermediate 10) to afford rac-2,2-difluoro-N-[(1S,2S)-2-methyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide (Example 111) as a white solid.

¹H NMR (400 MHz, DMSO-d₆) δ: ppm 11.09-10.83 (m, 1H), 8.75-8.63 (m, 1H), 7.75-7.71 (m, 1H), 7.19-6.57 (m, 1H), 4.30-4.09 (m, 2H), 3.91-3.80 (m, 2H), 3.76-3.49 (m, 2H), 3.22-2.94 (m, 2H), 2.76-2.55 (m, 2H), 2.23-2.09 (m, 1H), 1.94-1.70 (m, 4H), 1.66-1.44 (m, 3H), 1.14-1.00 (m, 4H), 0.90-0.79 (m, 9H).

MS obsd. (ESI⁺) [(M+H)⁺]: 578.3.

### Example 112

### N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide

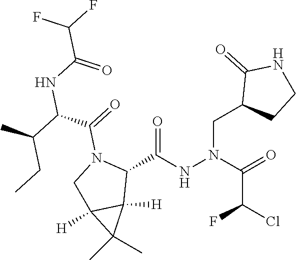 [SMILES: CC[C@@H](C)[C@H](NC(=O)C(F)F)C(=O)N1C[C@H]2[C@@H]([C@H]1C(=O)NN(C[C@@H]1CCNC1=O)C(=O)[C@H](F)Cl)C2(C)C]

### Step 1: Preparation of methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid

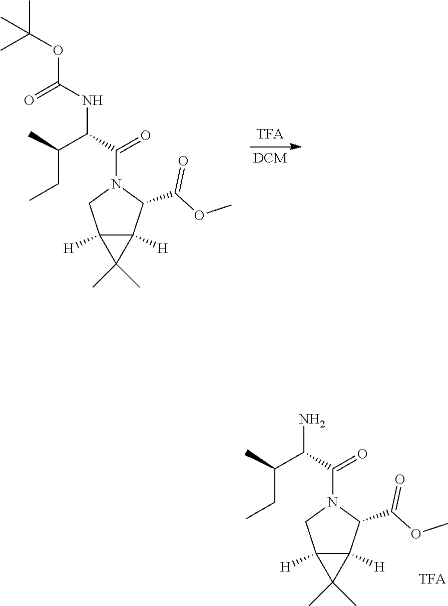 [SMILES: CC[C@@H](C)[C@H](NC(=O)OC(C)(C)C)C(=O)N1C[C@H]2[C@@H]([C@H]1C(=O)OC)C2(C)C ; CC[C@@H](C)[C@H](N)C(=O)N1C[C@H]2[C@@H]([C@H]1C(=O)OC)C2(C)C]

To a solution of methyl (1R,2S,5S)-3-[(2S,3R)-2-(tert-butoxycarbonylamino)-3-methyl-pentanoyl]-6,6-dimethyl-3-azabicyclo[3.1.0]hexane-2-carboxylate (3.1 g, 8.1 mmol, from Example 64, step 1) in DCM (20 mL) was added TFA (20.0 mL). The mixture was stirred at 25° C. for 30 min and concentrated in vacuum to afford methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid (3.2 g) as yellow oil.

MS obsd. (ESI⁺) [(M+H)⁺]: 283.0

### Step 2: Preparation of (1R,2S,5S)-3-[(2S,3R)-2-(benzyloxycarbonylamino)-3-methyl-pentanoyl]-6,6-dimethyl-3-azabicyclo[3.1.0]hexane-2-carboxylate

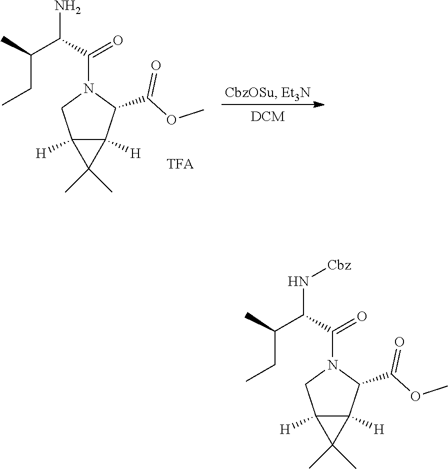

To a solution of methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid (3.2 g, 8.07 mmol) in THF (20 mL) was added Et₃N (2.46 g, 24.31 mmol) and CbzOSu (3.02 g, 12.13 mmol). The reaction mixture was stirred at 25° C. for 1 h and concentrated in vacuum. The residue was purified by silica gel column eluted with PE/EtOAc=10/1 to 3/1 to afford methyl (1R,2S,5S)-3-[(2S,3R)-2-(benzyloxycarbonylamino)-3-methyl-pentanoyl]-6,6-dimethyl-3-azabicyclo[3.1.0]hexane-2-carboxylate (3.3 g) as yellow oil.

MS obsd. (ESI⁺) [(M+H)⁺]: 417.2.

### Step 3: Preparation of (1R,2S,5S)-3-[(2S,3R)-2-(benzyloxycarbonylamino)-3-methyl-pentanoyl]-6,6-dimethyl-3-azabicyclo[3.1.0]hexane-2-carboxylic acid

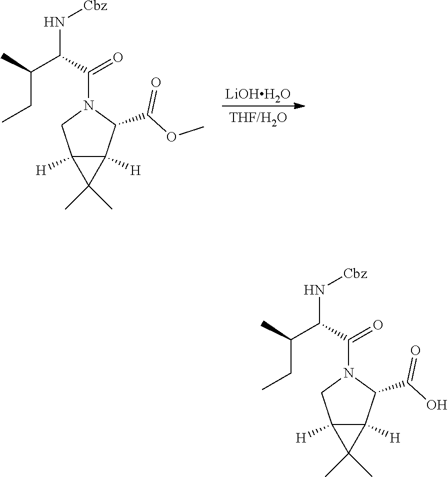

To a solution of methyl (1R,2S,5S)-3-[(2S,3R)-2-(benzyloxycarbonylamino)-3-methyl-pentanoyl]-6,6-dimethyl-3-azabicyclo[3.1.0]hexane-2-carboxylate (3.6 g, 8.64 mmol) in a mixed solvent of THF (15 mL) and water (15 mL) was added LiOH·H₂O (726 mg, 17.3 mmol) at 0° C. The reaction mixture was stirred for 2 h at 25° C. The resulting solution was acidified with 1 N HCl (50 mL) and extracted with EtOAc (50 mL×3). The organic phase was washed with brine (50 mL), dried over Na₂SO₄ and concentrated in vacuum to afford (1R,2S,5S)-3-[(2S,3R)-2-(benzyloxycarbonylamino)-3-methyl-pentanoyl]-6,6-dimethyl-3-azabicyclo[3.1.0]hexane-2-carboxylic acid (3.3 g) as colorless oil.

MS obsd. (ESI⁺) [(M+H)⁺]: 403.2.

### Step 4: Preparation of tert-butyl N-[[(1R,2S,5S)-3-[(2S,3R)-2-(benzyloxycarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

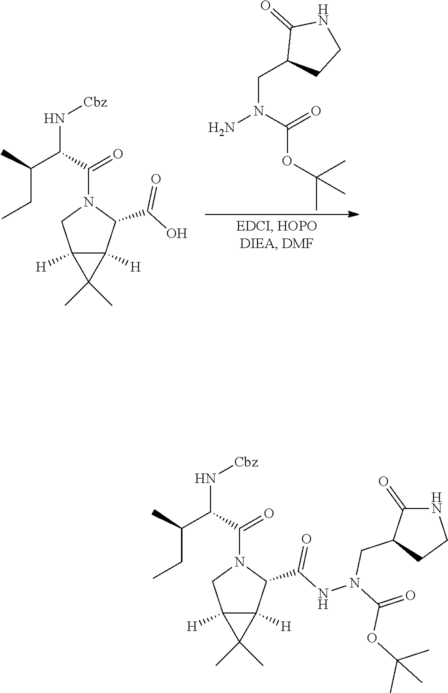 [SMILES: CC[C@@H](C)[C@H](NC(=O)OCc1ccccc1)C(=O)N1C[C@H]2[C@@H]([C@H]1C(=O)O)C2(C)C ; CC(C)(C)OC(=O)N(N)C[C@@H]1CCNC1=O ; CC[C@@H](C)[C@H](NC(=O)OCc1ccccc1)C(=O)N1C[C@H]2[C@@H]([C@H]1C(=O)NN(C[C@@H]1CCNC1=O)C(=O)OC(C)(C)C)C2(C)C]

To a solution of (1R,2S,5S)-3-[(2S,3R)-2-(benzyloxycarbonylamino)-3-methyl-pentanoyl]-6,6-dimethyl-3-azabicyclo[3.1.0]hexane-2-carboxylic acid (3.3 g, 8.2 mmol) in DMF (30 mL) was added DIPEA (5.3 g, 41.03 mmol). After cooling to 0° C., EDCI (1.89 g, 9.85 mmol), HOPO (1.1 g, 9.84 mmol) and tert-butyl N-amino-N-[[(3S)-2-oxopyrrolidin-3-yl]methyl]carbamate (1.89 g, 8.24 mmol) was added into the mixture. Then the reaction mixture was stirred at 30° C. for 12 h. The reaction mixture was poured into EtOAc (150 mL) and washed with 1 N HCl (50 mL), 5% aqueous solution of K₂CO₃ (50 mL) and brine (50 mL). The resulting organic phase was dried over anhydrous Na₂SO₄, filtered and concentrated in vacuum to afford tert-butyl N-[[(1R,2S,5S)-3-[(2S,3R)-2-(benzyloxycarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (4.68 g) as yellow oil.

MS obsd. (ESI⁺) [(M+H)⁺]: 614.4.

### Step 5: Preparation of tert-butyl N-[[(1R,2S,5S)-3-[(2S,3R)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

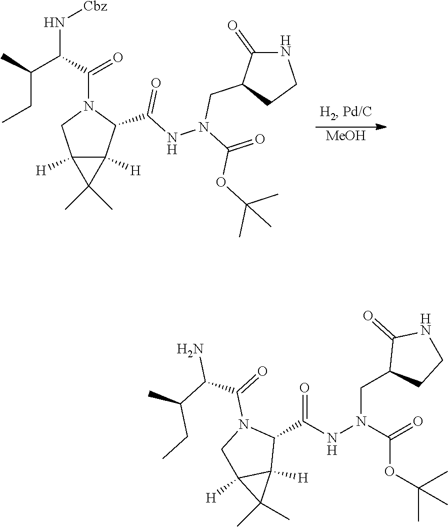 [SMILES: "MeOH" ; CC[C@@H](C)[C@H](NC(=O)OCc1ccccc1)C(=O)N1C[C@H]2[C@@H]([C@H]1C(=O)NN(C[C@@H]1CCNC1=O)C(=O)OC(C)(C)C)C2(C)C ; CC[C@@H](C)[C@H](N)C(=O)N1C[C@H]2[C@@H]([C@H]1C(=O)NN(C[C@@H]1CCNC1=O)C(=O)OC(C)(C)C)C2(C)C]

To a solution of tert-butyl N-[[(1R,2S,5S)-3-[(2S,3R)-2-(benzyloxycarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (4.68 g, 7.63 mmol) in methanol (50 mL) was added Pd/C (500 mg, 10% purity). The suspension was degassed under vacuum and purged with H₂ for three times. The resulting mixture was stirred at 25° C. for 1 h under a H₂ balloon. The suspension was filtered and the filtrate was concentrated in vacuum to afford tert-butyl N-[[(1R,2S,5S)-3-[(2S,3R)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (3.2 g) as a white solid.

MS obsd. (ESI⁺) [(M+H)⁺]: 480.3.

### Step 6: Preparation of tert-butyl N-[[(1R,2S,5S)-3-[(2S,3R)-2-[(2,2-difluoroacetyl)amino]-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

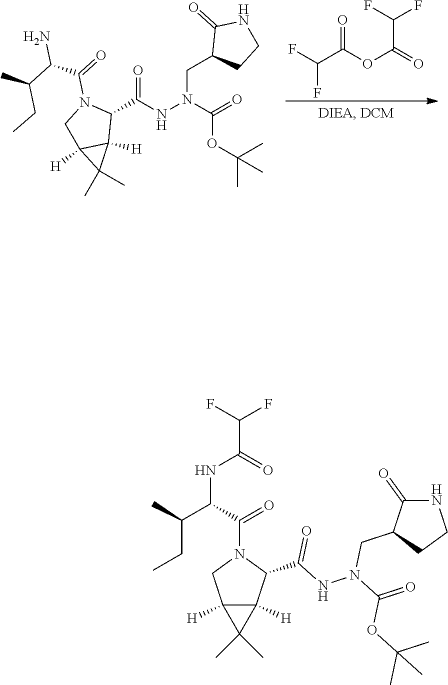

To a solution of tert-butyl N-[[(1R,2S,5S)-3-[(2S,3R)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (420 mg, 0.88 mmol) in DCM (10 mL) was added DIPEA (339 mg, 2.63 mmol) and (2,2-difluoroacetyl) 2,2-difluoroacetate (228 mg, 1.31 mmol). The mixture was stirred at 25° C. for 1 h. The mixture was diluted with DCM (50 mL), washed with brine (60 mL), dried over Na₂SO₄ and concentrated in vacuum to afford tert-butyl N-[[(1R,2S,5S)-3-[(2S,3R)-2-[(2,2-difluoroacetyl)amino]-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (488 mg) as yellow oil.

MS obsd. (ESI⁺) [(M+H)⁺]: 558.3.

### Step 7: Preparation of N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-2,2-difluoro-acetamide; hydrochloride

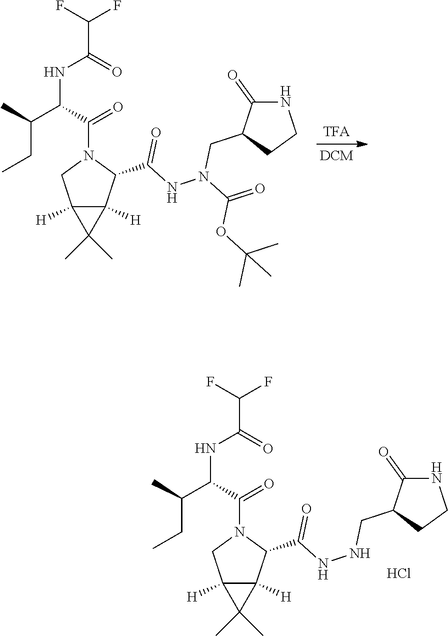 [SMILES: CC[C@@H](C)[C@H](NC(=O)C(F)F)C(=O)N1C[C@H]2[C@@H]([C@H]1C(=O)NN(C[C@@H]1CCNC1=O)C(=O)OC(C)(C)C)C2(C)C ; CC[C@@H](C)[C@H](NC(=O)C(F)F)C(=O)N1C[C@H]2[C@@H]([C@H]1C(=O)NNC[C@@H]1CCNC1=O)C2(C)C ; Cl]

A solution of tert-butyl N-[[(1R,2S,5S)-3-[(2S,3R)-2-[(2,2-difluoroacetyl)amino]-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (400 mg, 0.72 mmol) and TFA (2.0 mL) in DCM (4 mL) was stirred at 25° C. for 1 h. The solution was concentrated in vacuum. The residue was purified by reversed flash chromatography (condition: 120 g Flash Column Welch Ultimate XB_C18 20-40 μm; 120 A, water (0.1% HCl)-ACN, 0˜38%, 70 mL/min) to afford N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-2,2-difluoro-acetamide; hydrochloride (290 mg) as a white solid.

MS obsd. (ESI⁺) [(M+H)⁺]: 458.2.

### Step 8: Preparation of N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide

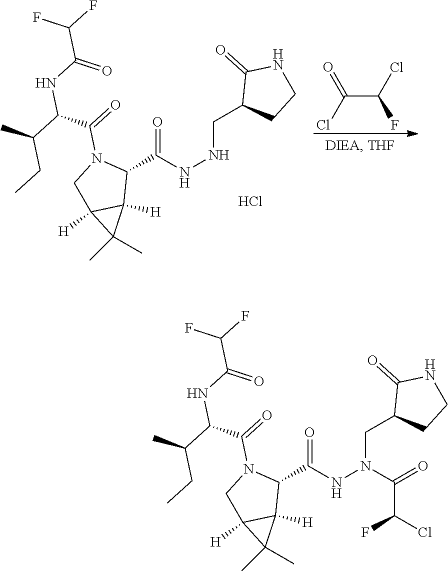

To a solution of N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]-2,2-difluoro-acetamide; hydrochloride (290 mg, 0.59 mmol) in THF (150 mL) was added DIPEA (1138 mg, 8.81 mmol). After cooling to 0° C., to the mixture was added (2R)-2-chloro-2-fluoro-acetyl chloride (345.89 mg, 1.06 mmol, 40% purity, Intermediate 10) in THF (10 mL). The resulting mixture was stirred at the same temperature for 1 h. The reaction was quenched with MeOH (10 mL) and acidified with 4 N HCl/dioxane until pH=5 at −10° C. The resulting mixture was concentrated in vacuum at 30° C. The residue was purified by reversed flash chromatography (condition: 120 g Flash Column Welch Ultimate XB_C18 20-40 μm; 120 A, water (0.1% FA)-ACN, 0˜53%, 70 mL/min) to afford N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide (238 mg, Example 112) as a white solid.

¹H NMR (400 MHz, DMSO-d₆) δ: ppm 11.16 (s, 1H), 9.13-9.08 (m, 1H), 7.75-7.70 (m, 1H), 6.80 (d, J=50.4 Hz, 1H), 6.24 (t, J=53.6 Hz, 1H), 4.38-4.29 (m, 1H), 4.17 (s, 1H), 3.91-3.75 (m, 3H), 3.44 (dd, J=13.2 Hz, 4.0 Hz, 1H), 3.22-3.10 (m, 2H), 2.63-2.58 (m, 1H), 2.20-2.09 (m, 1H), 1.95-1.75 (m, 2H), 1.68-1.60 (m, 1H), 1.53 (d, J=7.6 Hz, 1H), 1.45-1.35 (m, 1H), 1.16-1.08 (m, 1H), 1.06 (s, 3H), 0.92 (s, 3H), 0.87-0.80 (m, 6H).

MS obsd. (ESI⁺) [(M+H)⁺]: 552.3.

### Example 113

### Rac-2,2-difluoro-N-[(1S,2R)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide

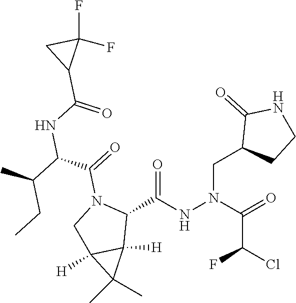 [SMILES: CC[C@@H](C)[C@H](NC(=O)C1CC1(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 112, by using 2,2-difluorocyclopropanecarboxylic acid and T₃P instead of (2,2-difluoroacetyl) 2,2-difluoroacetate to afford rac-2,2-difluoro-N-[(1S,2R)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide (Example 113) as a white solid.

¹H NMR (400 MHz, DMSO-d₆) δ: ppm 11.16-11.13 (m, 1H), 8.67-8.55 (m, 1H), 7.75-7.72 (m, 1H), 6.91-6.77 (m, 1H), 4.36 (t, J=8.4 Hz, 1H), 4.16 (s, 1H), 3.90-3.72 (m, 3H), 3.43 (dd, J=13.6 Hz, 4.0 Hz, 1H), 3.22-3.12 (m, 2H), 2.63-2.58 (m, 1H), 2.20-2.11 (m, 1H), 1.95-1.70 (m, 5H), 1.65-1.52 (m, 2H), 1.44-1.34 (m, 1H), 1.10-1.00 (m, 4H), 0.88 (d, J=10.0 Hz, 3H), 0.86-0.79 (m, 6H).

MS obsd. (ESI⁺) [(M+H)⁺]: 578.3.

### Example 114

### N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide

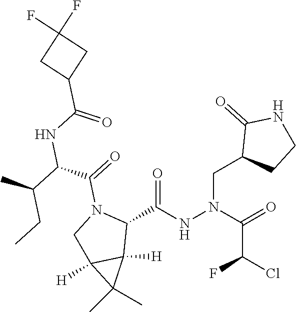 [SMILES: CC[C@@H](C)[C@H](NC(=O)C1CC(F)(F)C1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 112, by using 3,3-difluorocyclobutanecarboxylic acid and T₃P instead of (2,2-difluoroacetyl) 2,2-difluoroacetate to afford N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide (Example 114) as a white solid.

¹H NMR (400 MHz, DMSO-d₆) δ: ppm 11.13 (s, 1H), 8.34-8.27 (m, 1H), 7.75 (s, 1H), 6.84 (d, J=50.4 Hz, 1H), 4.32 (t, J=8.4 Hz, 1H), 4.15 (s, 1H), 3.90-3.75 (m, 3H), 3.44 (dd, J=13.6 Hz, 4.4 Hz, 1H), 3.24-3.08 (m, 2H), 3.05-2.94 (m, 1H), 2.74-2.59 (m, 5H), 2.25-2.10 (m, 1H), 1.85-1.70 (m, 2H), 1.67-1.57 (m, 1H), 1.51 (d, J=7.6 Hz, 1H), 1.42-1.30 (m, 1H), 1.05-0.96 (m, 4H), 0.91 (s, 3H), 0.87-0.74 (m, 6H).

MS obsd. (ESI⁺) [(M+H)⁺]: 592.3.

### Example 115

### N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide

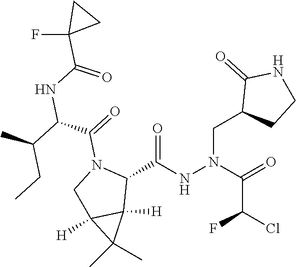 [SMILES: CC[C@@H](C)[C@H](NC(=O)C1(F)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 112, by using 1-fluorocyclopropane-1-carboxylic acid and T₃P instead of (2,2-difluoroacetyl) 2,2-difluoroacetate to afford N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide (Example 115) as a white solid.

¹H NMR (400 MHz, DMSO-d₆) δ: ppm 11.14 (s, 1H), 8.31-8.18 (m, 1H), 7.75 (s, 1H), 6.84 (d, J=50.4 Hz, 1H), 4.31 (t, J=8.0 Hz, 1H), 4.17 (s, 1H), 3.94-3.77 (m, 3H), 3.43 (dd, J=13.6 Hz, 4.4 Hz, 1H), 3.22-3.10 (m, 2H), 2.64-2.58 (m, 1H), 2.23-2.11 (m, 1H), 2.00-1.88 (m, 1H), 1.84-1.73 (m, 1H), 1.66-1.57 (m, 1H), 1.51 (d, J=7.6 Hz, 1H), 1.47-1.36 (m, 1H), 1.34-1.26 (m, 2H), 1.24-1.10 (m, 2H), 1.10-1.07 (m, 1H), 1.05 (s, 3H), 0.90 (s, 3H), 0.86-0.80 (m, 6H).

MS obsd. (ESI⁺) [(M+H)⁺]: 560.3.

### Example 116

### (2R)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide

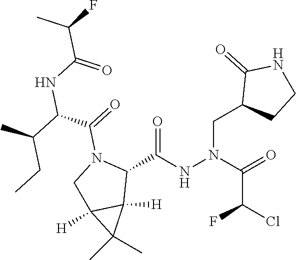 [SMILES: CC[C@@H](C)[C@H](NC(=O)[C@@H](C)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 112, by using (2R)-2-fluoropropanoic acid and T₃P instead of (2,2-difluoroacetyl) 2,2-difluoroacetate to afford (2R)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide (Example 116) as a white solid.

¹H NMR (400 MHz, DMSO-d₆) δ: ppm 11.17 (s, 1H), 8.35-8.27 (m, 1H), 7.77-7.70 (m, 1H), 6.83 (d, J=50.8 Hz, 1H), 5.19-4.93 (m, 1H), 4.35-4.25 (m, 1H), 4.16 (s, 1H), 3.91-3.79 (m, 3H), 3.44 (dd, J=14.0 Hz, 4.4 Hz, 1H), 3.21-3.10 (m, 2H), 2.62-2.57 (m, 1H), 2.21-2.15 (m, 1H), 1.88-1.70 (m, 2H), 1.68-1.57 (m, 1H), 1.53 (d, J=7.6 Hz, 1H), 1.45-1.31 (m, 4H), 1.12-0.99 (m, 4H), 0.92 (s, 3H), 0.85-0.79 (m, 6H).

MS obsd. (ESI⁺) [(M+H)⁺]: 548.4.

### Example 117

### (2S)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide

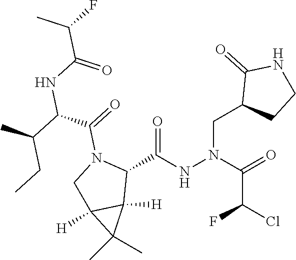 [SMILES: CC[C@@H](C)[C@H](NC(=O)[C@H](C)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 112, by using (2S′)-2-fluoropropanoic acid and T₃P instead of (2,2-difluoroacetyl) 2,2-difluoroacetate to afford (2S)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide (Example 117) as a white solid.

¹H NMR (400 MHz, DMSO-d₆) δ: ppm 11.16 (s, 1H) 8.31-8.15 (m, 1H), 7.70-7.67 (m, 1H), 6.81 (d, J=50.4 Hz, 1H), 5.11-4.92 (m, 1H), 4.31 (t, J=8.0 Hz, 1H), 4.15 (s, 1H), 3.89-3.72 (m, 3H), 3.43 (dd, J=14.0, 4.0 Hz, 1H), 3.21-3.09 (m, 2H), 2.62-2.54 (m, 1H), 2.19-2.08 (m, 1H), 1.87-1.70 (m, 2H), 1.65-1.59 (m, 1H), 1.51 (d, J=7.6 Hz, 1H), 1.37 (dd, J=24.8 Hz, 6.8 Hz, 4H), 1.10-0.97 (m, 4H), 0.92-0.86 (m, 3H), 0.85-0.78 (m, 6H).

MS obsd. (ESI⁺) [(M+H)+]: 548.4.

### Example 118

### N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide

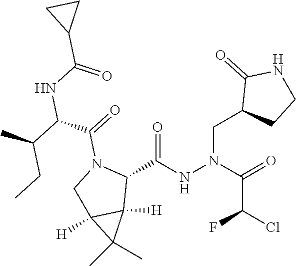 [SMILES: CC[C@@H](C)[C@H](NC(=O)C1CC1)C(=O)N1C[C@H]2[C@@H]([C@H]1C(=O)NN(C[C@@H]1CCNC1=O)C(=O)[C@H](F)Cl)C2(C)C]

### Step 1: Preparation of methyl (1R,2S,5S)-3-[(2S,3R)-2-(cyclopropanecarbonylamino)-3-methyl-pentanoyl]-6,6-dimethyl-3-azabicyclo[3.1.0]hexane-2-carboxylate

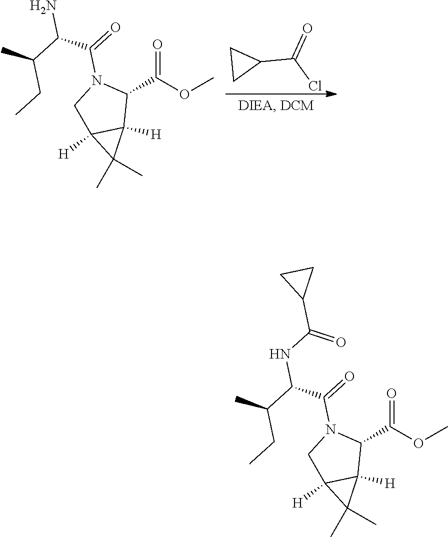

To a solution of methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid (660 mg, 2.07 mmol, Intermediate 29) in DCM (10 mL) was added DIPEA (1337 mg, 10.35 mmol) and cyclopropanecarbonyl chloride (259 mg, 2.48 mmol). The mixture was stirred at 25° C. for 1 h. The reaction mixture was diluted with DCM (200 mL), washed with brine (60 mL×3) and dried over Na₂SO₄ and concentrated in vacuum to afford methyl (1R,2S,5S)-3-[(2S,3R)-2-(cyclopropanecarbonylamino)-3-methyl-pentanoyl]-6,6-dimethyl-3-azabicyclo[3.1.0]hexane-2-carboxylate (725 mg) as yellow foam.

MS obsd. (ESI⁺) [(M+H)⁺]: 351.1.

### Step 2: Preparation of (1R,2S,5S)-3-[(2S,3R)-2-(cyclopropanecarbonylamino)-3-methyl-pentanoyl]-6,6-dimethyl-3-azabicyclo[3.1.0]hexane-2-carboxylic acid

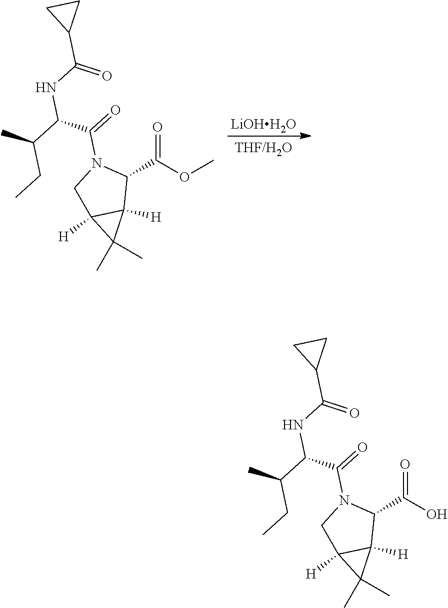

To a solution of methyl (1R,2S,5S)-3-[(2S,3R)-2-(cyclopropanecarbonylamino)-3-methyl-pentanoyl]-6,6-dimethyl-3-azabicyclo[3.1.0]hexane-2-carboxylate (700 mg, 2.0 mmol) in THF (10 mL) and water (5 mL) was added LiOH·H₂O (251 mg, 5.99 mmol). The mixture was stirred at 25° C. for 1 h. The reaction mixture was acidified with 1 N HCl to pH=5. The resulting mixture was extracted with EtOAc (50 mL×3). The combined organic phase was washed with brine (60 mL), dried over Na₂SO₄ and concentrated in vacuum to afford (1R,2S,5S)-3-[(2S,3R)-2-(cyclopropanecarbonylamino)-3-methyl-pentanoyl]-6,6-dimethyl-3-azabicyclo[3.1.0]hexane-2-carboxylic acid (660 mg) as a yellow solid.

### Step 3: Preparation of tert-butyl N-[[(1R,2S,5S)-3-[(2S,3R)-2-(cyclopropanecarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate

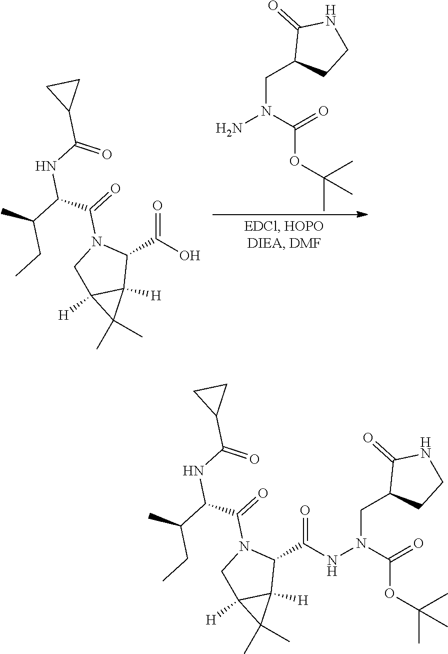 [SMILES: CC[C@@H](C)[C@H](NC(=O)C1CC1)C(=O)N1C[C@H]2[C@@H]([C@H]1C(=O)O)C2(C)C ; CC(C)(C)OC(=O)N(N)C[C@@H]1CCNC1=O ; CC[C@@H](C)[C@H](NC(=O)C1CC1)C(=O)N1C[C@H]2[C@@H]([C@H]1C(=O)NN(C[C@@H]1CCNC1=O)C(=O)OC(C)(C)C)C2(C)C]

To a solution of (1R,2S,5S)-3-[(2S,3R)-2-(cyclopropanecarbonylamino)-3-methyl-pentanoyl]-6,6-dimethyl-3-azabicyclo[3.1.0]hexane-2-carboxylic acid (670 mg, 1.99 mmol) in DMF (10 mL) was added DIPEA (772 mg, 5.97 mmol), HOPO (288 mg, 2.59 mmol), EDCI (496 mg, 2.59 mmol) and tert-butyl N-amino-N-[[(3S)-2-oxopyrrolidin-3-yl]methyl]carbamate (547.93 mg, 2.39 mmol, Intermediate 9) at 0° C. The mixture was stirred at 25° C. for 12 h. The reaction mixture was diluted with EtOAc (200 mL), washed with 1 N HCl (30 mL) and brine (60 mL×2). The organic phase was dried over Na₂SO₄, filtered and concentrated in vacuum to afford tert-butyl N-[[(1R,2S,5S)-3-[(2S,3R)-2-(cyclopropanecarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (650 mg) as a yellow solid.

MS obsd. (ESI⁺) [(M+H)⁺]: 548.2.

### Step 4: Preparation of N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]cyclopropanecarboxamide

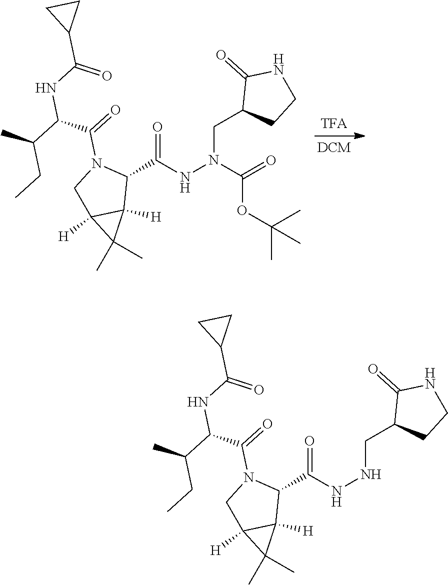

To a solution of tert-butyl N-[[(1R,2S,5S)-3-[(2S,3R)-2-(cyclopropanecarbonylamino)-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (690 mg, 1.26 mmol) in DCM (4 mL) was added TFA (2.0 mL). The mixture was stirred at 25° C. for 1 h. The mixture was concentrated under vacuum to give a residue. The residue was purified by reversed flash chromatography (condition: 120 g Flash Column Welch Ultimate XB_C18 20-40 μm; 120 A, water (0.1% HCl)-ACN, 0˜40%, 55 mL/min) to afford N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]cyclopropanecarboxamide; hydrochloride (430 mg) as a white solid.

MS obsd. (ESI⁺) [(M+H)⁺]: 448.1.

### Step 5: Preparation of N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide

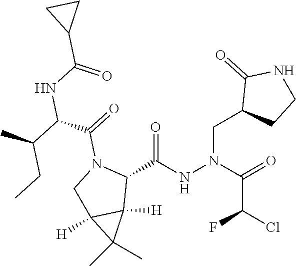 [SMILES: CC[C@@H](C)[C@H](NC(=O)C1CC1)C(=O)N1C[C@H]2[C@@H]([C@H]1C(=O)NN(C[C@@H]1CCNC1=O)C(=O)[C@H](F)Cl)C2(C)C]

To a solution of N-[(1S,2R)-1-[(1R,2S,5S)-6,6-dimethyl-2-[[[(3S)-2-oxopyrrolidin-3-yl]methylamino]carbamoyl]-3-azabicyclo[3.1.0]hexane-3-carbonyl]-2-methyl-butyl]cyclopropanecarboxamide; hydrochloride (350 mg, 0.72 mmol) in THF (120 mL) was added DIPEA (935 mg, 7.23 mmol). After cooling to −5° C., to the mixture was added (2R)-2-chloro-2-fluoro-acetyl chloride (403 mg, 1.23 mmol, 40% purity) in THF (10 mL). The reaction mixture was stirred at the same temperature for 30 min. The reaction was quenched with MeOH (10 mL) and acidified by 4 N HCl/dioxane to pH=5 at −10° C. Then the mixture was concentrated in vacuum at 30° C. and the residue was purified by reversed flash chromatography (condition: 120 g Flash Column Welch Ultimate XB_C18 20-40 μm; 120 A, water (0.1% FA)-ACN, 0˜49%, 55 mL/min) to afford N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide (375.92 mg, Example 118) as a white solid.

¹H NMR (400 MHz, DMSO-d6) δ: ppm 11.11 (s, 1H), 8.33-8.20 (m, 1H) 7.75-7.63 (m, 1H), 6.85 (d, J=50.4 Hz, 1H), 4.28 (t, J=8.4 Hz, 1H), 4.13 (s, 1H), 3.85-3.75 (m, 3H), 3.43 (dd, J=12.8, 3.2 Hz, 1H), 3.17-3.09 (m, 2H), 2.62-2.56 (m, 1H), 2.20-2.10 (m, 1H), 1.82-1.65 (m, 3H), 1.62-1.55 (m, 1H), 1.49-1.47 (m, 1H), 1.42-1.32 (m, 1H), 1.09-0.98 (m, 4H), 0.88-0.78 (m, 9H), 0.71-0.52 (m, 4H).

MS obsd. (ESI⁺) [(M+H)⁺]: 542.3.

### Example 119

### N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide

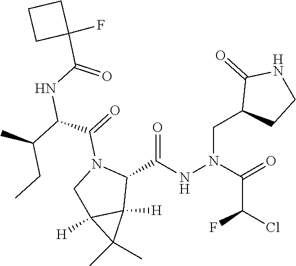 [SMILES: CC[C@@H](C)[C@H](NC(=O)C1(F)CCC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 112, by using 1-fluorocyclobutanecarboxylic acid and T₃P instead of (2,2-difluoroacetyl) 2,2-difluoroacetate to afford N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide (Example 119) as a white solid.

¹H NMR (400 MHz, DMSO-d₆) δ: ppm 11.17 (s, 1H), 8.11-7.97 (m, 1H), 7.78 (s, 1H), 6.83 (d, J=50.8 Hz, 1H), 4.30 (t, J=8.4 Hz, 1H), 4.15 (s, 1H), 3.97-3.89 (m, 1H), 3.87-3.84 (m, 1H), 3.84-3.75 (m, 1H), 3.45 (dd, J=14.0 Hz, 4.0 Hz, 1H), 3.20-3.13 (m, 2H), 2.67-2.55 (m, 1H), 2.50-2.48 (m, 1H), 2.47-2.41 (m, 1H), 2.40-2.27 (m, 3H), 2.19-2.11 (m, 1H), 1.95-1.85 (m, 2H), 1.81-1.61 (m, 3H), 1.52 (d, J=7.6 Hz, 1H), 1.44-1.30 (m, 1H), 1.08-0.96 (m, 4H), 0.88 (s, 3H), 0.87-0.75 (m, 5H).

MS obsd. (ESI⁺) [(M+H)⁺]: 574.4.

### Example 120

### N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide

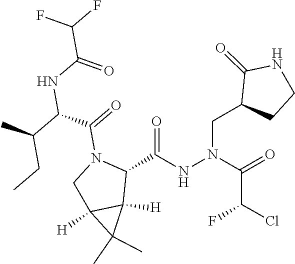 [SMILES: CC[C@@H](C)[C@H](NC(=O)C(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 112, by using (2S)-2-chloro-2-fluoro-acetyl chloride (Intermediate 28) instead of (2R)-2-chloro-2-fluoro-acetyl chloride (Intermediate 10) to afford N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide (Example 120) as a white solid.

¹H NMR (400 MHz, DMSO-d₆) δ: ppm 11.05-10.82 (m, 1H), 9.14-9.09 (m, 1H), 7.75-7.71 (m, 1H), 7.11-6.56 (m, 1H), 6.22 (t, J=53.6 Hz, 1H), 4.37-4.26 (m, 1H), 4.21-4.13 (m, 1H), 3.89-3.65 (m, 3H), 3.54-3.50 (m, 1H), 3.20-2.98 (m, 2H), 2.65-2.56 (m, 1H), 2.22-2.09 (m, 1H), 1.87-1.73 (m, 2H), 1.62-1.43 (m, 3H), 1.15-1.10 (m, 4H), 0.90-0.78 (m, 9H).

MS obsd. (ESI⁺) [(M+H)⁺]: 552.3.

### Example 121

### N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide

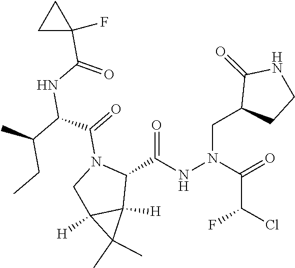 [SMILES: CC[C@@H](C)[C@H](NC(=O)C1(F)CC1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 112, by using 1-fluorocyclopropane-1-carboxylic acid and T₃P instead of (2,2-difluoroacetyl) 2,2-difluoroacetate, and (2S)-2-chloro-2-fluoro-acetyl chloride (Intermediate 28) instead of (2R)-2-chloro-2-fluoro-acetyl chloride (Intermediate 10) to afford N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide (Example 121) as a white solid.

¹H NMR (400 MHz, DMSO-d₆) δ: ppm 11.03-10.82 (m, 1H), 8.27 (dd, J=21.2 Hz, 6.8 Hz, 1H), 7.77-7.70 (m, 1H), 7.13-6.56 (m, 1H), 4.34-4.26 (m, 1H), 4.21-4.13 (m, 1H), 3.91-3.80 (m, 2H), 3.79-3.63 (m, 1H), 3.57-3.48 (m, 1H), 3.20-3.00 (m, 2H), 2.67-2.57 (m, 1H), 2.21-2.13 (m, 1H), 1.97-1.87 (m, 1H), 1.84-1.72 (m, 1H), 1.60-1.43 (m, 3H), 1.33-1.24 (m, 2H), 1.23-1.15 (m, 1H), 1.10-1.01 (m, 5H), 0.95-0.75 (m, 9H).

MS obsd. (ESI⁺) [(M+H)⁺]: 560.3.

### Example 122

### (2S)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide

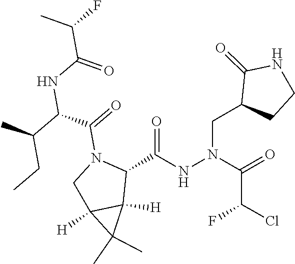 [SMILES: CC[C@@H](C)[C@H](NC(=O)[C@H](C)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 112, by using (2S)-2-fluoropropanoic acid and T₃P instead of (2,2-difluoroacetyl) 2,2-difluoroacetate, and (2S)-2-chloro-2-fluoro-acetyl chloride (Intermediate 28) instead of (2R)-2-chloro-2-fluoro-acetyl chloride (Intermediate 10) to afford (2S)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide (Example 122) as a white solid.

¹H NMR (400 MHz, DMSO-d₆) δ: ppm 11.03-10.83 (m, 1H), 8.28-8.10 (m, 1H), 7.82-7.65 (m, 1H), 7.11-6.56 (m, 1H), 5.11-4.92 (m, 1H), 4.35-4.26 (m, 1H), 4.23-4.10 (m, 1H), 3.89-3.79 (m, 2H), 3.75-3.62 (m, 1H), 3.57-3.48 (m, 1H), 3.21-2.98 (m, 2H), 2.65-2.53 (m, 1H), 2.22-2.10 (m, 1H), 1.88-1.74 (m, 2H), 1.61-1.48 (m, 3H), 1.45 (dd, J=29.6 Hz, 6.4 Hz, 3H), 1.10-1.00 (m, 4H), 0.89 (s, 3H), 0.85-0.75 (m, 6H).

MS obsd. (ESI⁺) [(M+H)+]: 548.3.

### Example 123

### (2R)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide

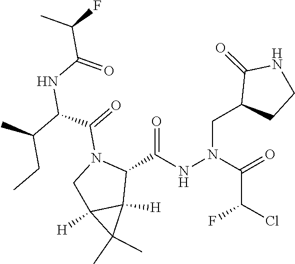 [SMILES: CC[C@@H](C)[C@H](NC(=O)[C@@H](C)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 112, by using (2R)-2-fluoropropanoic acid and T₃P instead of (2,2-difluoroacetyl) 2,2-difluoroacetate, and (2S)-2-chloro-2-fluoro-acetyl chloride (Intermediate 28) instead of (2R)-2-chloro-2-fluoro-acetyl chloride (Intermediate 10) to afford (2R)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide (Example 123) as a white solid.

¹H NMR (400 MHz, DMSO-d₆) δ: ppm 11.11-10.76 (m, 1H), 8.38-8.24 (m, 1H), 7.78-7.71 (m, 1H), 7.13-6.56 (m, 1H), 5.14-4.94 (m, 1H), 4.35-4.12 (m, 2H), 3.87-3.64 (m, 3H), 3.57-3.42 (m, 1H), 3.21-2.98 (m, 2H), 2.65-2.55 (m, 1H), 2.20-2.10 (m, 1H), 1.87-1.73 (m, 2H), 1.68-1.42 (m, 3H), 1.36 (dd, J=24.4 Hz, 6.4 Hz, 3H), 1.11-0.98 (m, 4H), 0.91-0.81 (m, 9H).

MS obsd. (ESI⁺) [(M+H)⁺]: 548.3.

### Example 124

### Rac-2,2-difluoro-N-[(1S,2R)-2-methyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide

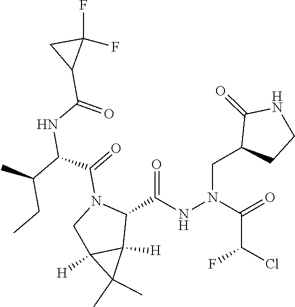 [SMILES: CC[C@@H](C)[C@H](NC(=O)C1CC1(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 112, by using 2,2-difluorocyclopropanecarboxylic acid and T₃P instead of (2,2-difluoroacetyl) 2,2-difluoroacetate, and (2S)-2-chloro-2-fluoro-acetyl chloride (Intermediate 28) instead of (2R)-2-chloro-2-fluoro-acetyl chloride (Intermediate 10) to afford rac-2,2-difluoro-N-[(1S,2R)-2-methyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide (Example 124) as a white solid.

¹H NMR (400 MHz, DMSO-d₆) δ: ppm 11.06-10.83 (m, 1H), 8.67-8.49 (m, 1H), 7.77-7.71 (m, 1H), 7.14-6.55 (m, 1H), 4.42-4.30 (m, 1H), 4.21-4.12 (m, 1H), 3.87-3.64 (m, 3H), 3.59-3.46 (m, 1H), 3.21-2.95 (m, 2H), 2.75-2.67 (m, 1H), 2.60-2.52 (m, 1H), 2.23-2.07 (m, 1H), 1.94-1.70 (m, 4H), 1.66-1.49 (m, 2H), 1.45-1.31 (m, 1H), 1.12-0.98 (m, 4H), 0.91-0.89 (m, 1H), 0.88-0.75 (m, 8H).

MS obsd. (ESI⁺) [(M+H)⁺]: 578.3.

### Example 125

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclobutanecarboxamide

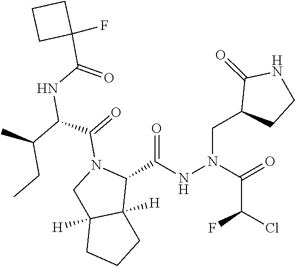 [SMILES: CC[C@@H](C)[C@H](NC(=O)C1(F)CCC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 105, by using methyl (3S,3aS,6aR)-1,2,3,3a,4,5,6,6a-octahydrocyclopenta[c]pyrrole-3-carboxylate; 2,2,2-trifluoroacetic acid (Intermediate 21) instead of methyl (1R,2S,5S)-6,6-dimethyl-3-azabicyclo[3.1.0]hexane-2-carboxylate; hydrochloride to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclobutanecarboxamide (Example 125) as a white solid.

¹H NMR (400 MHz, DMSO-d₆) δ: ppm 11.05 (s, 1H), 8.14-7.97 (m, 1H), 7.77-7.71 (m, 1H), 6.94 (d, J=50.4 Hz, 1H), 4.30 (t, J=8.8 Hz, 1H), 4.07-3.97 (m, 1H), 3.87-3.80 (m, 2H), 3.78-3.71 (m, 1H), 3.39 (dd, J=14.0 Hz, 4.0 Hz, 1H), 3.22-3.10 (m, 2H), 2.76-2.70 (m, 1H), 2.60-2.55 (m, 1H), 2.45-2.23 (m, 5H), 2.19-2.15 (m, 1H), 1.93-1.78 (m, 4H), 1.77-1.58 (m, 5H), 1.49-1.34 (m, 2H), 1.09-0.99 (m, 1H), 0.84-0.77 (m, 6H).

MS obsd. (ESI⁺) [(M+H)⁺]: 574.2.

### Example 126

### N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide

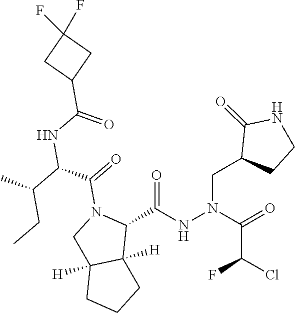 [SMILES: CC[C@H](C)[C@H](NC(=O)C1CC(F)(F)C1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 105, by using methyl (3S,3aS,6aR)-1,2,3,3a,4,5,6,6a-octahydrocyclopenta[c]pyrrole-3-carboxylate; 2,2,2-trifluoroacetic acid (Intermediate 21) instead of methyl (1R,2S,5S)-6,6-dimethyl-3-azabicyclo[3.1.0]hexane-2-carboxylate; hydrochloride, and 3,3-difluorocyclobutanecarboxylic acid instead of 1-fluorocyclobutanecarboxylic acid to afford N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide (Example 126) as a white solid.

¹H NMR (400 MHz, DMSO-d₆) δ: ppm 11.04 (s, 1H), 8.44-8.42 (m, 1H), 7.76 (s, 1H), 6.96 (d, J=50.4 Hz, 1H), 4.28 (t, J=8.8 Hz, 1H), 3.94 (d, J=6.4 Hz, 1H), 3.85-3.81 (m, 2H), 3.73 (dd, J=14.0 Hz, 10.0 Hz, 1H), 3.39 (dd, J=13.6 Hz, 4.0 Hz, 1H), 3.21-3.10 (m, 2H), 3.00-2.94 (m, 1H), 2.78-2.68 (m, 2H), 2.67-2.62 (m, 2H), 2.60-2.53 (m, 2H), 2.52-2.51 (m, 1H), 2.19-2.10 (m, 1H), 1.91-1.77 (m, 2H), 1.76-1.66 (m, 4H), 1.65-1.58 (m, 1H), 1.49-1.34 (m, 2H), 1.12-1.03 (m, 1H), 0.85-0.76 (m, 6H).

MS obsd. (ESI⁺) [(M+H)⁺]: 592.3.

### Example 127

### N-[(1S,2S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide

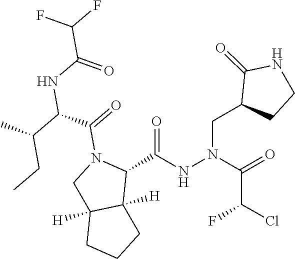 [SMILES: CC[C@H](C)[C@H](NC(=O)C(F)F)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 105, by using methyl (3S,3aS,6aR)-1,2,3,3a,4,5,6,6a-octahydrocyclopenta[c]pyrrole-3-carboxylate; 2,2,2-trifluoroacetic acid (Intermediate 21) instead of methyl (1R,2S,5S)-6,6-dimethyl-3-azabicyclo[3.1.0]hexane-2-carboxylate; hydrochloride and (2S)-2-chloro-2-fluoro-acetyl chloride (Intermediate 28) instead of (2R)-2-chloro-2-fluoro-acetyl chloride (Intermediate 10), and (2,2-difluoroacetyl) 2,2-difluoroacetate without coupling reagent instead of 1-fluorocyclobutanecarboxylic acid to afford N-[(1S,2S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide (Example 127) as a white solid.

¹H NMR (400 MHz, DMSO-d₆) δ: ppm 11.03-10.73 (m, 1H), 9.23-9.17 (m, 1H), 7.76-7.72 (m, 1H), 7.19-6.43 (m, 1H), 6.22 (t, J=53.6 Hz, 1H), 4.39-4.27 (m, 1H), 4.05-4.00 (m, 1H), 3.87-3.68 (m, 3H), 3.56-3.47 (m, 1H), 3.19-2.97 (m, 2H), 2.81-2.68 (m, 2H), 2.64-2.52 (m, 1H), 2.24-2.09 (m, 1H), 1.84-1.61 (m, 7H), 1.53-1.33 (m, 2H), 1.14-1.02 (m, 1H), 0.88 (d, J=6.8 Hz, 3H), 0.81 (t, J=7.2 Hz, 3H).

MS obsd. (ESI⁺) [(M+H)⁺]: 552.2.

### Example 128

### N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide

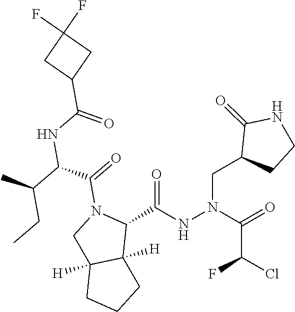 [SMILES: CC[C@@H](C)[C@H](NC(=O)C1CC(F)(F)C1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using benzyl (3S,3aS,6aR)-2-[(2S,3R)-2-amino-3-methyl-pentanoyl]-3,3a,4,5,6,6a-hexahydro-1H-cyclopenta[c]pyrrole-3-carboxylate; 2,2,2-trifluoroacetic acid (Intermediate 22) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), 3,3-difluorocyclobutanecarboxylic acid and T₃P instead of TFAA to afford N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide (Example 128) as a white solid.

¹H NMR (400 MHz, DMSO-d₆) δ: ppm 11.07 (s, 1H), 8.32-8.20 (m, 1H), 7.77 (s, 1H), 6.91 (d, J=50.4 Hz, 1H), 4.46 (t, J=7.6 Hz, 1H), 4.10-3.93 (m, 1H), 3.86-3.78 (m, 1H), 3.77-3.69 (m, 1H), 3.39 (dd, J=13.6 Hz, 4.4 Hz, 1H), 3.23-3.11 (m, 2H), 3.08-3.01 (m, 1H), 2.80-2.59 (m, 6H), 2.55-2.52 (m, 1H), 2.20-2.10 (m, 1H), 1.92-1.59 (m, 8H), 1.44-1.33 (m, 2H), 1.10-0.98 (m, 1H), 0.88-0.78 (m, 6H).

MS obsd. (ESI⁺) [(M+H)⁺]: 592.2.

### Example 129

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]cyclopropanecarboxamide

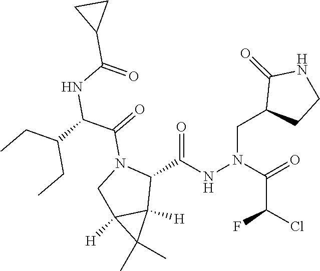 [SMILES: CCC(CC)[C@H](NC(=O)C1CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 118, by using methyl (1R,2S,5S)-3-[(2S)-2-amino-3-ethyl-pentanoyl]-6,6-dimethyl-3-azabicyclo[3.1.0]hexane-2-carboxylate; 2,2,2-trifluoroacetic acid (Intermediate 30) instead of methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid (Intermediate 29) to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]cyclopropanecarboxamide (Example 129) as a white solid.

¹H NMR (400 MHz, DMSO-d₆) δ: ppm 11.12 (s, 1H), 8.36-8.22 (m, 1H), 7.75 (s, 1H), 6.87 (d, J=50.4 Hz, 1H), 4.41 (t, J=8.8 Hz, 1H), 4.14 (s, 1H), 3.91 (d, J=10.4 Hz, 1H), 3.85-3.75 (m, 2H), 3.43 (dd, J=14.0 Hz, 4.4 Hz, 1H), 3.22-3.12 (m, 2H), 2.61-2.56 (m, 1H), 2.20-2.10 (m, 1H), 1.84-1.74 (m, 1H), 1.72-1.58 (m, 3H), 1.52-1.39 (m, 2H), 1.35-1.20 (m, 3H), 1.04 (s, 3H), 0.88 (s, 3H), 0.83-0.73 (m, 6H), 0.70-0.60 (m, 3H), 0.59-0.50 (m, 1H).

MS obsd. (ESI⁺) [(M+H)⁺]: 556.3.

### Example 130

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-3,3-difluoro-cyclobutanecarboxamide

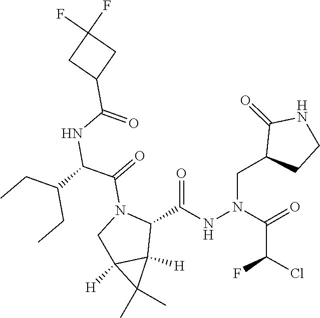 [SMILES: CCC(CC)[C@H](NC(=O)C1CC(F)(F)C1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 118, by using methyl (1R,2S,5S)-3-[(2S)-2-amino-3-ethyl-pentanoyl]-6,6-dimethyl-3-azabicyclo[3.1.0]hexane-2-carboxylate; 2,2,2-trifluoroacetic acid (Intermediate 30) instead of methyl (1R,2S,5S)-3-[(2S,3R)-2-amino-3-methyl-pentanoyl]-6,6-dimethyl-3-azabicyclo[3.1.0]hexane-2-carboxylate; 2,2,2-trifluoroacetic acid (Intermediate 29), and 3,3-difluorocyclobutanecarboxylic acid and T₃P instead of cyclopropanecarbonyl chloride to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-3,3-difluoro-cyclobutanecarboxamide (Example 130) as a white solid.

¹H NMR (400 MHz, DMSO-d₆) δ: ppm 11.14 (s, 1H), 8.41-8.35 (m, 1H), 7.77-7.72 (m, 1H), 6.80 (d, J=50.4 Hz, 1H), 4.41 (t, J=8.8 Hz, 1H), 4.19-4.13 (m, 1H), 3.91-3.75 (m, 3H), 3.43 (dd, J=14.0 Hz, 4.4 Hz, 1H), 3.21-3.10 (m, 2H), 3.00-2.91 (m, 1H), 2.73-2.53 (m, 5H), 2.18-2.10 (m, 1H), 1.81-1.72 (m, 1H), 1.69-1.60 (m, 2H), 1.50 (d, J=7.6 Hz, 1H), 1.43-1.32 (m, 1H), 1.31-1.14 (m, 3H), 1.04 (s, 3H), 0.89-0.86 (m, 3H), 0.80-0.71 (m, 6H).

MS obsd. (ESI⁺) [(M+H)⁺]: 606.3.

### Example 131

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2-difluoro-acetamide

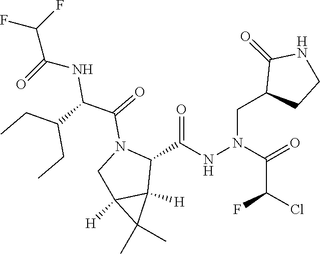 [SMILES: CCC(CC)[C@H](NC(=O)C(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 15, by using benzyl (1R,2S,5S)-3-[(2S)-2-amino-3-ethyl-pentanoyl]-6,6-dimethyl-3-azabicyclo[3.1.0]hexane-2-carboxylate; hydrochloride (Intermediate 31) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and (2,2-difluoroacetyl) 2,2-difluoroacetate without coupling reagent instead of bicyclo[1.1.1]pentane-1-carboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2-difluoro-acetamide (Example 131) as a white solid.

¹H NMR (400 MHz, DMSO-d₆) δ: ppm 11.16 (s, 1H), 9.18-9.07 (m, 1H), 7.75 (s, 1H), 6.92 (d, J=50.8 Hz, 1H), 6.22 (t, J=53.6 Hz, 1H), 4.42 (t, J=8.8 Hz, 1H), 4.15 (s, 1H), 3.88-3.76 (m, 3H), 3.43 (dd, J=14.0 Hz, 4.4 Hz, 1H), 3.19-3.12 (m, 2H), 2.61-2.55 (m, 1H), 2.17-2.10 (m, 1H), 1.80-1.70 (m, 2H), 1.65-1.58 (m, 1H), 1.53-1.51 (m, 1H), 1.44-1.36 (m, 1H), 1.30-1.17 (m, 3H), 1.04 (s, 3H), 0.89 (s, 3H), 0.79 (t, J=7.2 Hz, 3H), 0.74 (t, J=7.2 Hz, 3H).

MS obsd. (ESI⁺) [(M+H)⁺]: 566.3.

### Example 132

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide

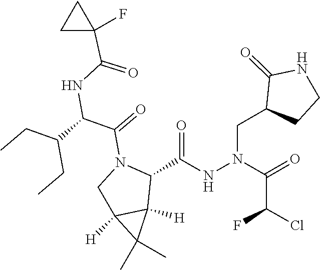 [SMILES: CCC(CC)[C@H](NC(=O)C1(F)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 15, by using benzyl (1R,2S,5S)-3-[(2S)-2-amino-3-ethyl-pentanoyl]-6,6-dimethyl-3-azabicyclo[3.1.0]hexane-2-carboxylate; hydrochloride (Intermediate 31) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid instead of bicyclo[1.1.1]pentane-1-carboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide (Example 132) as a white solid.

¹H NMR (400 MHz, DMSO-d₆) δ: ppm 11.15 (s, 1H), 8.38 (d, J=8.4 Hz, 1H), 7.76 (s, 1H), 6.82 (d, J=50.8 Hz, 1H), 4.44 (t, J=8.8 Hz, 1H), 4.15 (s, 1H), 3.98-3.93 (m, 1H), 3.85-3.77 (m, 2H), 3.42 (dd, J=13.6 Hz, 2.8 Hz, 1H), 3.21-3.07 (m, 2H), 2.61-2.55 (m, 1H), 2.19-2.11 (m, 1H), 1.93-1.83 (m, 1H), 1.82-1.70 (m, 1H), 1.65-1.55 (m, 1H), 1.52-1.48 (m, 1H), 1.46-1.34 (m, 1H), 1.33-1.15 (m, 6H), 1.12-1.01 (m, 4H), 0.89-0.85 (m, 3H), 0.83-0.71 (m, 6H).

MS obsd. (ESI⁺) [(M+H)⁺]: 574.2.

### Example 133

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide

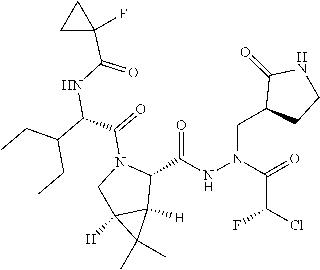 [SMILES: CCC(CC)[C@H](NC(=O)C1(F)CC1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 15, by using benzyl (1R,2S,5S)-3-[(2S)-2-amino-3-ethyl-pentanoyl]-6,6-dimethyl-3-azabicyclo[3.1.0]hexane-2-carboxylate; hydrochloride (Intermediate 31) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), (2S)-2-chloro-2-fluoro-acetyl chloride (Intermediate 28) instead of (2R)-2-chloro-2-fluoro-acetyl chloride (Intermediate 10), and 1-fluorocyclopropane-1-carboxylic acid instead of bicyclo[1.1.1]pentane-1-carboxylic acid to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide (Example 133) as a white solid.

¹H NMR (400 MHz, DMSO-d₆) δ: ppm 11.05-10.83 (m, 1H), 8.36-8.25 (m, 1H), 7.82-7.61 (m, 1H), 7.12-6.56 (m, 1H), 4.49-4.40 (m, 1H), 4.24-4.11 (m, 1H), 3.98-3.88 (m, 1H), 3.87-3.62 (m, 2H), 3.58-3.39 (m, 1H), 3.21-2.96 (m, 2H), 2.64-2.51 (m, 1H), 2.23-2.11 (m, 1H), 1.97-1.72 (m, 2H), 1.64-1.38 (m, 3H), 1.35-1.14 (m, 6H), 1.11-1.01 (m, 4H), 0.81 (s, 3H), 0.84-0.71 (m, 6H).

MS obsd. (ESI⁺) [(M+H)⁺]: 574.3.

### Example 134

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-3,3-difluoro-cyclobutanecarboxamide

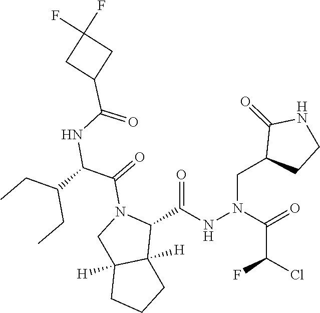 [SMILES: CCC(CC)[C@H](NC(=O)C1CC(F)(F)C1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 15, by using benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 3,3-difluorocyclobutanecarboxylic acid instead of bicyclo[1.1.1]pentane-1-carboxylic acid to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-3,3-difluoro-cyclobutanecarboxamide (Example 134) as a white solid.

¹H NMR (400 MHz, DMSO-d₆) δ: ppm 11.06 (s, 1H), 8.35-8.24 (m, 1H), 7.71-7.69 (m, 1H), 6.86 (d, J=50.4 Hz, 1H), 4.51 (t, J=8.4 Hz, 1H), 3.96 (d, J=5.6 Hz, 1H), 3.85-3.71 (m, 3H), 3.38 (dd, J=14.0 Hz, 4.0 Hz, 1H), 3.22-3.11 (m, 2H), 3.05-2.96 (m, 1H), 2.75-2.54 (m, 7H), 2.18-2.11 (m, 1H), 1.91-1.78 (m, 2H), 1.76-1.68 (m, 3H), 1.66-1.61 (m, 2H), 1.43-1.30 (m, 2H), 1.29-1.14 (m, 3H), 0.85-0.71 (m, 6H).

MS obsd. (ESI⁺) [(M+H)⁺]: 606.4.

### Example 135

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]cyclopropanecarboxamide

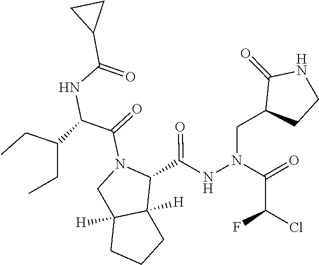 [SMILES: CCC(CC)[C@H](NC(=O)C1CC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 15, by using benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and cyclopropanecarbonyl chloride without coupling reagent instead of bicyclo[1.1.1]pentane-1-carboxylic acid to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]cyclopropanecarboxamide (Example 135) as a white solid.

¹H NMR (400 MHz, DMSO-d₆) δ: ppm 11.07 (s, 1H), 8.36-8.29 (m, 1H), 7.77 (s, 1H), 6.94 (d, J=50.4 Hz, 1H), 4.50 (t, J=8.8 Hz, 1H), 3.96 (d, J=6.0 Hz, 1H), 3.84-3.69 (m, 3H), 3.41-3.36 (m, 1H), 3.23-3.10 (m, 2H), 2.78-2.69 (m, 1H), 2.56-2.53 (m, 1H), 2.22-2.07 (m, 1H), 1.89-1.77 (m, 2H), 1.77-1.68 (m, 4H), 1.67-1.58 (m, 2H), 1.49-1.38 (m, 1H), 1.37-1.30 (m, 1H), 1.28-1.18 (m, 3H), 0.90-0.69 (m, 7H), 0.67-0.55 (m, 4H).

MS obsd. (ESI⁺) [(M+H)⁺]: 556.3.

### Example 136

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide

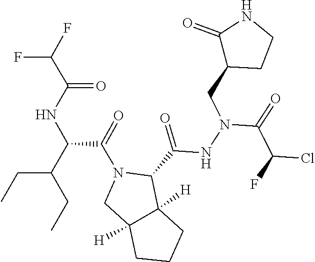 [SMILES: CCC(CC)[C@H](NC(=O)C(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 15, by using benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and (2,2-difluoroacetyl) 2,2-difluoroacetate without coupling reagent instead of bicyclo[1.1.1]pentane-1-carboxylic acid to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide (Example 136) as a white solid.

¹H NMR (400 MHz, DMSO-d₆) δ: ppm 11.10 (s, 1H), 9.13-9.03 (m, 1H), 7.77-7.74 (m, 1H), 6.90 (d, J=50.4 Hz, 1H), 6.25 (t, J=53.6 Hz, 1H), 4.54 (t, J=8.0 Hz, 1H), 3.99 (d, J=5.6 Hz, 1H), 3.87-3.67 (m, 3H), 3.39 (dd, J=14.0 Hz, 4.0 Hz, 1H), 3.19-3.10 (m, 2H), 2.82-2.70 (m, 1H), 2.60-2.54 (m, 1H), 2.19-2.10 (m, 1H), 1.90-1.78 (m, 2H), 1.77-1.67 (m, 4H), 1.66-1.57 (m, 1H), 1.46-1.35 (m, 2H), 1.31-1.14 (m, 4H), 0.82 (t, J=7.2 Hz, 3H), 0.75 (t, J=7.6 Hz, 3H).

MS obsd. (ESI⁺) [(M+H)⁺]: 566.3.

### Example 137

### Rac-2-fluoro-N-[(1S)-2-ethyl-1-[(2S)-4,4-dimethyl-2-[[[(2R)-2-chloro-2-fluoro-acetyl]-[[(3S)-2-oxopyrrolidin-3-yl]methyl]amino]carbamoyl]pyrrolidine-1-carbonyl]butyl]propanamide

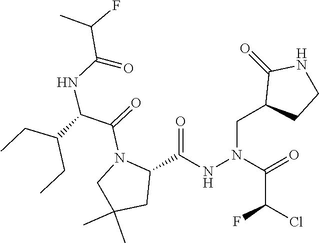 [SMILES: CCC(CC)[C@H](NC(=O)C(C)F)C(=O)N1CC(C)(C)C[C@H]1C(=O)NN(C[C@@H]1CCNC1=O)C(=O)[C@H](F)Cl]

The title compound was prepared in analogy to the procedure described for the preparation of Example 15, by using benzyl (2S)-1-[(2S)-2-amino-3-ethyl-pentanoyl]-4,4-dimethyl-pyrrolidine-2-carboxylate; hydrochloride (Intermediate 32) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 2-fluoropropionic acid instead of bicyclo[1.1.1]pentane-1-carboxylic acid to afford rac-2-fluoro-N-[(1S)-2-ethyl-1-[(2S)-4,4-dimethyl-2-[[[(2R)-2-chloro-2-fluoro-acetyl]-[[(3S)-2-oxopyrrolidin-3-yl]methyl]amino]carbamoyl]pyrrolidine-1-carbonyl]butyl]propanamide (Example 137) as a white solid.

¹H NMR (400 MHz, DMSO-d₆) δ: ppm 11.00 (s, 1H), 8.29 (dd, J=33.6 Hz, 8.4 Hz, 1H), 7.75-7.71 (m, 1H), 6.95 (d, J=50.8 Hz, 1H), 5.15-5.07 (m, 1H), 4.49 (q, J=8.0 Hz, 1H), 4.33-4.27 (m, 1H), 3.82-3.68 (m, 2H), 3.40 (dd, J=13.6 Hz, 4.0 Hz, 1H), 3.29-3.27 (m, 2H), 3.19-3.10 (m, 2H), 2.16-2.07 (m, 1H), 1.97-1.92 (m, 1H), 1.78-1.61 (m, 3H), 1.41 (dd, J=6.4 Hz, 2.0 Hz, 2H), 1.35 (dd, J=6.8 Hz, 2.0 Hz, 2H), 1.31-1.18 (m, 3H), 1.14 (s, 3H), 0.99 (d, J=4.4 Hz, 3H), 0.83-0.72 (m, 6H).

MS obsd. (ESI⁺) [(M+H)⁺]: 550.3.

### Example 138

### N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-1-fluoro-cyclopropanecarboxamide

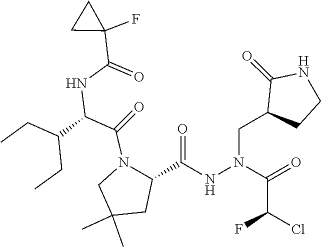 [SMILES: CCC(CC)[C@H](NC(=O)C1(F)CC1)C(=O)N1CC(C)(C)C[C@H]1C(=O)NN(C[C@@H]1CCNC1=O)C(=O)[C@H](F)Cl]

The title compound was prepared in analogy to the procedure described for the preparation of Example 15, by using benzyl (2S)-1-[(2S)-2-amino-3-ethyl-pentanoyl]-4,4-dimethyl-pyrrolidine-2-carboxylate; hydrochloride (Intermediate 32) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid instead of bicyclo[1.1.1]pentane-1-carboxylic acid to afford N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-1-fluoro-cyclopropanecarboxamide (Example 138) as a white solid.

¹H NMR (400 MHz, DMSO-d₆) δ: ppm 10.99 (s, 1H), 8.28 (d, J=8.0 Hz, 1H), 7.75 (s, 1H), 7.00 (d, J=50.4 Hz, 1H), 4.50 (t, J=8.4 Hz, 1H), 4.31 (d, J=10.0 Hz, 7.2 Hz, 1H), 3.84 (d, J=9.6 Hz, 1H), 3.72 (dd, J=14.0 Hz, 10.0 Hz, 1H), 3.41 (dd, J=13.6 Hz, 4.0 Hz, 1H), 3.26 (d, J=9.6 Hz, 1H), 3.20-3.10 (m, 2H), 2.17-2.08 (m, 1H), 1.98-1.92 (m, 1H), 1.90-1.81 (m, 1H), 1.79-1.61 (m, 2H), 1.45-1.17 (m, 9H), 1.13 (s, 3H), 0.97 (s, 3H), 0.81 (t, J=7.6 Hz, 3H), 0.75 (t, J=7.6 Hz, 3H).

MS obsd. (ESI⁺) [(M+H)⁺]: 562.2.

### Example 139

### N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-2,2-difluoro-acetamide

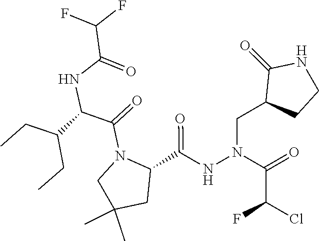 [SMILES: CCC(CC)[C@H](NC(=O)C(F)F)C(=O)N1CC(C)(C)C[C@H]1C(=O)NN(C[C@@H]1CCNC1=O)C(=O)[C@H](F)Cl]

The title compound was prepared in analogy to the procedure described for the preparation of Example 15, by using benzyl (2S)-1-[(2S)-2-amino-3-ethyl-pentanoyl]-4,4-dimethyl-pyrrolidine-2-carboxylate; hydrochloride (Intermediate 32) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and (2,2-difluoroacetyl) 2,2-difluoroacetate without coupling reagent instead of bicyclo[1.1.1]pentane-1-carboxylic acid to afford N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-2,2-difluoro-acetamide (Example 139) as a white solid.

¹H NMR (400 MHz, DMSO-d₆) δ: ppm 11.17-10.45 (m, 1H), 9.25-9.00 (m, 1H), 7.75 (s, 1H), 7.13-6.90 (m, 1H), 6.25 (t, J=24 Hz, 1H), 4.57-4.46 (m, 1H), 4.37-4.27 (m, 1H), 3.80-3.64 (m, 2H), 3.40 (dd, J₁=4 Hz, J₂=16 Hz 1H), 3.31-3.20 (m, 1H), 3.21-3.07 (m, 2H), 2.54-2.51 (br s, 1H), 2.31-2.05 (m, 1H), 2.03-1.92 (m, 1H), 1.80-1.59 (m, 3H), 1.47-1.38 (m, 1H), 1.34-1.20 (m, 3H), 1.15 (s, 3H), 1.00 (s, 3H), 0.86-0.73 (m, 6H).

MS obsd. (ESI⁺) [(M+H)⁺]: 554.2.

### Example 140

### (2S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide

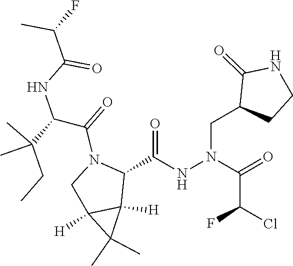 [SMILES: CCC(C)(C)[C@H](NC(=O)[C@H](C)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, and (2S)-2-fluoropropanoic acid instead of 3,3-difluorocyclobutanecarboxylic acid to afford (2S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide (Example 140) as a white solid.

¹H NMR (400 MHz, DMSO-d₆) δ: ppm 11.17 (s, 1H), 7.96-7.87 (m, 1H), 7.76-7.68 (m, 1H), 6.86 (d, J=50.4 Hz, 1H), 5.19-5.03 (m, 1H), 4.46 (d, J=8.8 Hz, 1H), 4.14 (s, 1H), 3.95-3.90 (m, 1H), 3.83-3.76 (m, 2H), 3.46 (dd, J=14.0, 4.0 Hz, 1H), 3.22-3.10 (m, 2H), 2.62-2.56 (m, 1H), 2.20-2.11 (m, 1H), 1.81-1.72 (m, 1H), 1.66-1.60 (m, 1H), 1.53-1.51 (m, 1H), 1.39-1.23 (m, 5H), 1.05 (s, 3H), 0.92-0.88 (m, 9H) 0.78 (t, J=7.2 Hz, 3H).

MS obsd. (ESI⁺) [(M+Na)⁺]: 584.2.

### Example 141

### (2R)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide

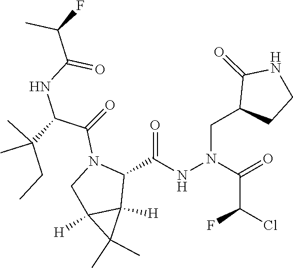 [SMILES: CCC(C)(C)[C@H](NC(=O)[C@@H](C)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) (S)-2-(((benzyloxy)carbonyl)amino)-3,3-dimethylbutanoic acid, and (2R)-2-fluoropropanoic acid instead of 3,3-difluorocyclobutanecarboxylic acid to afford (2R)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide (Example 141) as a white solid.

¹H NMR (400 MHz, DMSO-d₆) δ: ppm 11.19 (s, 1H), 8.11-7.98 (m, 1H), 7.79 (s, 1H), 6.89 (dd, J=50.8 Hz, 1H), 5.21-5.07 (m, 1H), 4.41 (d, J=8.8 Hz, 1H), 4.14 (s, 1H), 3.95-3.89 (m, 1H), 3.83-3.75 (m, 2H), 3.45 (dd, J=13.6 Hz, 4.0 Hz, 1H), 3.23-3.10 (m, 2H), 2.63-2.57 (m, 1H), 2.25-2.09 (m, 1H), 1.85-1.70 (m, 1H), 1.67-1.58 (m, 1H), 1.53 (d, J=7.6 Hz, 1H), 1.37 (dd, J=24.4 Hz, 6.4 Hz, 3H), 1.31-1.18 (m, 2H), 1.05 (s, 3H), 0.93-0.84 (m, 9H), 0.76 (t, J=6.8 Hz, 3H).

MS obsd. (ESI⁺) [(M+H)⁺]: 562.3.

### Example 142

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide

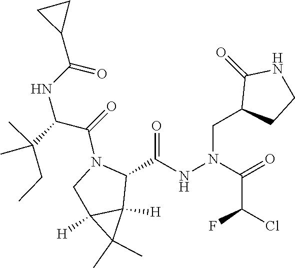 [SMILES: CCC(C)(C)[C@H](NC(=O)C1CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, and cyclopropanecarbonyl chloride without coupling reagent instead of 3,3-difluorocyclobutanecarboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide (Example 142) as a white solid.

¹H NMR (400 MHz, DMSO-d₆) δ: ppm 11.13 (s, 1H), 8.17-8.09 (m, 1H), 7.75-7.70 (m, 1H), 6.92 (d, J=50.4 Hz, 1H), 4.43 (d, J=9.2 Hz, 1H), 4.11 (s, 1H), 3.85-3.76 (m, 3H), 3.44 (dd, J=14.0 Hz, 4.4 Hz, 1H), 3.18-3.13 (m, 2H), 2.62-2.56 (m, 1H), 2.18-2.10 (m, 1H), 1.87-1.71 (m, 2H), 1.62-1.57 (m, 1H), 1.48 (d, J=7.6 Hz, 1H), 1.35-1.20 (m, 2H), 1.03 (s, 3H), 0.90-0.83 (m, 9H), 0.75 (t, J=7.6 Hz, 3H), 0.68-0.59 (m, 3H) 0.57-0.51 (m, 1H).

MS obsd. (ESI⁺) [(M+H)⁺]: 556.3.

### Example 143

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide

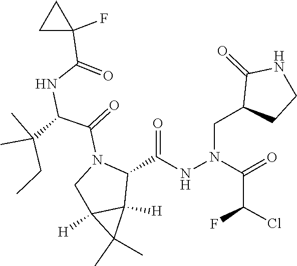 [SMILES: CCC(C)(C)[C@H](NC(=O)C1(F)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, and 1-fluorocyclopropane-1-carboxylic acid instead of 3,3-difluorocyclobutanecarboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide (Example 143) as a white solid.

¹H NMR (400 MHz, DMSO-d₆) δ: ppm 11.17 (s, 1H), 7.75-7.71 (m, 1H), 7.47-7.35 (m, 1H), 6.84 (d, J=50.4 Hz, 1H), 4.48 (d, J=8.8 Hz, 1H), 4.24-4.16 (m, 1H), 3.94-3.89 (m, 1H), 3.83-3.76 (m, 2H), 3.45 (dd, J=14.0 Hz, 4.4 Hz, 1H), 3.22-3.09 (m, 2H), 2.62-2.57 (m, 1H), 2.20-2.11 (m, 1H), 1.82-1.72 (m, 1H), 1.64-1.57 (m, 1H), 1.52 (d, J=7.6 Hz, 1H), 1.39-1.19 (m, 5H), 1.11-1.03 (m, 4H), 0.97-0.90 (m, 6H), 0.89-0.83 (m, 3H), 0.78 (t, J=7.6 Hz, 3H).

MS obsd. (ESI⁺) [(M+H)⁺]: 574.3.

### Example 144

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide

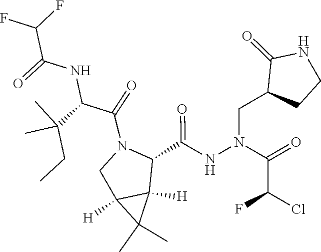 [SMILES: CCC(C)(C)[C@H](NC(=O)C(F)F)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, and (2,2-difluoroacetyl) 2,2-difluoroacetate without coupling reagent instead of 3,3-difluorocyclobutanecarboxylic acid to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide (Example 144) as a white solid.

¹H NMR (400 MHz, CDCl₃) δ: ppm 10.02 (br. s, 1H), 7.10-7.03 (m, 1H), 6.66 (d, J=50.8 Hz, 1H), 6.22 (br. s, 1H), 5.94 (t, J=54.0 Hz, 1H), 4.67 (d, J=10.0 Hz, 1H), 4.50-4.41 (m, 2H), 4.07-4.03 (m, 1H), 3.94-3.91 (m, 1H), 3.44-3.40 (m, 2H), 2.94-2.80 (m, 1H), 2.43-2.35 (m, 1H), 1.86-1.66 (m, 2H), 1.38-1.25 (m, 4H), 1.07 (s, 3H), 0.99 (s, 3H), 0.96 (s, 3H), 0.91 (s, 3H), 0.87 (t, J=7.6 Hz, 3H).

MS obsd. (ESI⁺) [(M+H)⁺]: 566.2.

### Example 145

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide

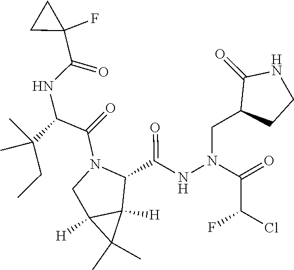 [SMILES: CCC(C)(C)[C@H](NC(=O)C1(F)CC1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, and (2S)-2-chloro-2-fluoro-acetyl chloride (Intermediate 28) instead of (2R)-2-chloro-2-fluoro-acetyl chloride (Intermediate 10), and 1-fluorocyclopropane-1-carboxylic acid instead of 3,3-difluorocyclobutanecarboxylic acid to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide (Example 145) as a white solid.

¹H NMR (400 MHz, DMSO-d₆) δ: ppm 11.03-10.82 (m, 1H), 8.27 (dd, J=21.2 Hz, 6.8 Hz, 1H), 7.77-7.70 (m, 1H), 7.13-6.56 (m, 1H), 4.34-4.26 (m, 1H), 4.21-4.13 (m, 1H), 3.91-3.80 (m, 2H), 3.79-3.63 (m, 1H), 3.57-3.48 (m, 1H), 3.20-3.00 (m, 2H), 2.67-2.57 (m, 1H), 2.21-2.13 (m, 1H), 1.97-1.87 (m, 1H), 1.84-1.72 (m, 1H), 1.60-1.43 (m, 3H), 1.33-1.24 (m, 2H), 1.23-1.15 (m, 1H), 1.10-1.01 (m, 5H), 0.95-0.75 (m, 9H).

MS obsd. (ESI⁺) [(M+H)⁺]: 560.3.

### Example 146

### Rac-2-fluoro-N-[(1S)-2,2-dimethyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide

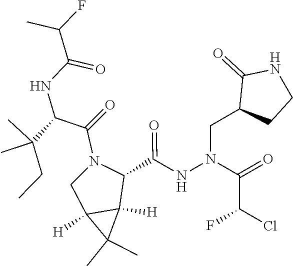 [SMILES: CCC(C)(C)[C@H](NC(=O)C(C)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of methyl (S)-2-(((benzyloxy)carbonyl)amino)-3,3-dimethylbutanoic acid, (2S)-2-chloro-2-fluoro-acetyl chloride (Intermediate 28) instead of (2R)-2-chloro-2-fluoro-acetyl chloride (Intermediate 10), and 2-fluoropropanoic acid instead of 3,3-difluorocyclobutanecarboxylic acid to afford rac-2-fluoro-N-[(1S)-2,2-dimethyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide (Example 146) as a white solid.

¹H NMR (400 MHz, DMSO-d₆) δ: ppm 11.15-10.84 (m, 1H), 7.98-7.88 (m, 1H), 7.75-7.69 (m, 1H), 7.15-6.55 (m, 1H), 5.21-5.01 (m, 1H), 4.50-4.39 (m, 1H), 4.24-4.09 (m, 1H), 3.92-3.86 (m, 1H), 3.81-3.68 (m, 2H), 3.56-3.46 (m, 1H), 3.21-2.98 (m, 2H), 2.60-2.55 (m, 1H), 2.21-2.09 (m, 1H), 1.84-1.72 (m, 1H), 1.65-1.46 (m, 2H), 1.40-1.19 (m, 5H), 1.04 (s, 3H), 0.94-0.83 (m, 9H), 0.80-0.72 (m, 3H).

MS obsd. (ESI⁺) [(M+H)⁺]: 562.3.

### Example 147

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide

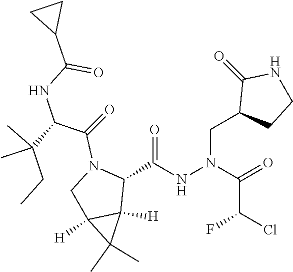 [SMILES: CCC(C)(C)[C@H](NC(=O)C1CC1)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, (2S)-2-chloro-2-fluoro-acetyl chloride (Intermediate 28) instead of (2R)-2-chloro-2-fluoro-acetyl chloride (Intermediate 10), and cyclopropanecarbonyl chloride without coupling reagent instead of 3,3-difluorocyclobutanecarboxylic acid to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide (Example 147) as a white solid.

¹H NMR (400 MHz, DMSO-d₆) δ: ppm 11.06-10.82 (m, 1H), 8.13 (d, J=9.2 Hz, 1H), 7.77-7.69 (m, 1H), 7.15-6.55 (m, 1H), 4.48-4.42 (m, 1H), 4.20-4.09 (m, 1H), 3.85-3.67 (m, 3H), 3.57-3.49 (m, 1H), 3.21-2.96 (m, 2H), 2.77-2.55 (m, 1H), 2.22-2.08 (m, 1H), 1.89-1.72 (m, 2H), 1.63-1.50 (m, 2H), 1.48-1.20 (m, 2H), 1.07-1.01 (m, 3H), 0.95-0.87 (m, 6H), 0.84 (s, 3H), 0.80-0.71 (m, 3H), 0.70-0.48 (m, 4H).

MS obsd. (ESI⁺) [(M+H)⁺]: 556.3.

### Example 148

### N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide

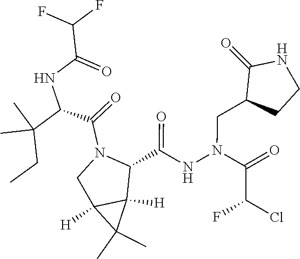 [SMILES: CCC(C)(C)[C@H](NC(=O)C(F)F)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 90, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, (2S)-2-chloro-2-fluoro-acetyl chloride (Intermediate 28) instead of (2R)-2-chloro-2-fluoro-acetyl chloride (Intermediate 10), and (2,2-difluoroacetyl) 2,2-difluoroacetate without coupling reagent instead of 3,3-difluorocyclobutanecarboxylic acid to afford N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide (Example 148) as a white solid.

¹H NMR (400 MHz, DMSO-d₆) δ: ppm 11.11-10.88 (m, 1H), 8.91 (d, J=8.8 Hz, 1H), 7.78-7.71 (m, 1H), 7.11-6.57 (m, 1H), 6.42-6.13 (m, 1H), 4.45-4.41 (m, 1H), 4.21-4.13 (m, 1H), 3.95-3.89 (m, 1H), 3.80-3.69 (m, 2H), 3.56-3.52 (m, 1H), 3.21-2.98 (m, 2H), 2.23-2.08 (m, 1H), 2.63-2.61 (m, 1H), 1.84-1.72 (m, 1H), 1.66-1.48 (m, 2H), 1.36-1.22 (m, 2H), 1.07-1.02 (m, 3H), 0.94-0.85 (m, 9H), 0.77 (t, J=7.2 Hz, 3H).

MS obsd. (ESI⁺) [(M+H)⁺]: 566.3.

### Example 149

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-1-fluoro-cyclopropanecarboxamide

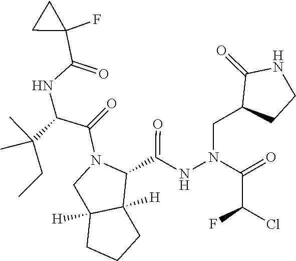 [SMILES: CCC(C)(C)[C@H](NC(=O)C1(F)CC1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid and T₃P instead of TFAA to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-1-fluoro-cyclopropanecarboxamide (Example 149) as a white solid.

¹H NMR (400 MHz, DMSO-d₆) δ: ppm 11.12 (s, 1H), 7.77-7.70 (s, 1H), 7.42-7.34 (m, 1H), 6.96 (d, J=50.4 Hz, 1H), 4.56 (d, J=8.8 Hz, 1H), 4.07-3.97 (m, 1H), 3.87 (dd, J=10.4 Hz, 7.2 Hz, 1H), 3.77-3.68 (m, 2H), 3.41 (dd, J=14.0 Hz, 4.0 Hz, 1H), 3.20-3.11 (m, 2H), 2.76-2.69 (m, 1H), 2.59-2.54 (m, 1H), 2.20-2.11 (m, 1H), 1.88-1.53 (m, 6H), 1.40-1.17 (m, 8H), 0.94 (s, 3H), 0.90 (s, 3H), 0.79 (t, J=7.6 Hz, 3H).

MS obsd. (ESI⁺) [(M+H)⁺]: 574.4.

### Example 150

### (1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide

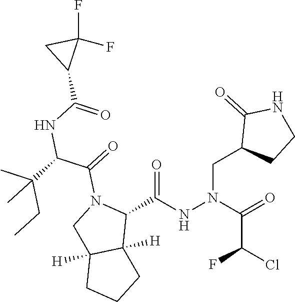 [SMILES: CCC(C)(C)[C@H](NC(=O)[C@@H]1CC1(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (JR, 2S,5S)-3-[(2S)-2-amino-3,3-dimethyl-butanoyl]-6,6-dimethyl-3-azabicyclo[3.1.0]hexane-2-carboxylate (Intermediate 1), and 2,2-difluorocyclopropanecarboxylic acid and T₃P instead of TFAA to afford (1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide (Example 150) as a white solid.

¹H NMR (400 MHz, DMSO-d₆) δ: ppm 11.10 (s, 1H), 8.50-8.44 (m, 1H), 7.78-7.72 (m, 1H), 7.03 (d, J=50.8 Hz, 1H), 4.53 (d, J=8.4 Hz, 1H), 3.95 (d, J=6.8 Hz, 1H), 3.90-3.81 (m, 1H), 3.78-3.67 (m, 2H), 3.42-3.37 (m, 1H), 3.21-3.12 (m, 2H), 2.95-2.84 (m, 1H), 2.78-2.71 (m, 1H), 2.58-2.53 (m, 1H), 2.20-2.12 (m, 1H), 1.92-1.81 (m, 3H), 1.80-1.69 (m, 4H), 1.67-1.60 (m, 1H), 1.40-1.30 (m, 2H), 1.29-1.14 (m, 2H), 0.93 (s, 3H), 0.90 (s, 3H), 0.77 (t, J=7.6 Hz, 3H).

MS obsd. (ESI⁺) [(M+H)⁺]: 592.1.

### Example 151

### N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide

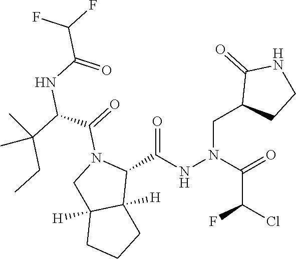 [SMILES: CCC(C)(C)[C@H](NC(=O)C(F)F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 1, by using (2S)-2-(benzyloxycarbonylamino)-3,3-dimethyl-pentanoic acid (Intermediate 33) instead of (S)-2-(((benzyloxy)carbonyl)amino)-3,3-dimethylbutanoic acid, benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (JR, 2S,5S)-3-[(2S)-2-amino-3,3-dimethyl-butanoyl]-6,6-dimethyl-3-azabicyclo[3.1.0]hexane-2-carboxylate (Intermediate 1), and (2,2-difluoroacetyl) 2,2-difluoroacetate instead of TFAA to afford N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide (Example 151) as a white solid.

¹H NMR (400 MHz, DMSO-d₆) δ: ppm 11.25 (s, 1H), 8.95-8.86 (m, 1H), 7.78-7.69 (m, 1H), 6.98 (d, J=50.8 Hz, 1H), 6.31 (t, J=53.6 Hz, 1H), 4.53 (d, J=8.4 Hz, 1H), 3.95 (d, J=6.4 Hz, 1H), 3.90-3.86 (m, 1H), 3.78-3.60 (m, 3H), 3.20-3.11 (m, 2H), 2.77-2.70 (m, 1H), 2.60-2.53 (m, 1H), 2.19-2.12 (m, 1H), 1.91-1.64 (m, 6H), 1.40-1.30 (m, 2H), 1.28-1.19 (m, 2H), 0.93 (s, 3H), 0.89 (s, 3H), 0.76 (t, J=7.2 Hz, 3H).

MS obsd. (ESI⁺) [(M+H)⁺]: 566.3.

### Example 152

### (1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide

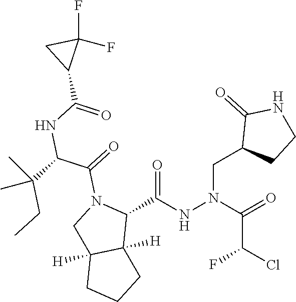 [SMILES: CCC(C)(C)[C@H](NC(=O)[C@@H]1CC1(F)F)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 1, by using benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), (2S)-2-chloro-2-fluoro-acetyl chloride (Intermediate 28) instead of (2R)-2-chloro-2-fluoro-acetyl chloride (Intermediate 10), and 2,2-difluorocyclopropanecarboxylic acid and T₃P instead of TFAA to afford (1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide (Example 152) as a white solid.

¹H NMR (400 MHz, DMSO-d₆) δ: ppm 11.06-10.73 (m, 1H), 8.45 (t, J=8.8 Hz, 1H), 7.77-7.70 (m, 1H), 7.22-6.40 (m, 1H), 4.53 (d, J=8.8 Hz, 1H), 3.99-3.94 (m 1H), 3.88-3.69 (m, 3H), 3.53-3.44 (m, 1H), 3.26-3.01 (m, 2H), 2.97-2.81 (m, 1H), 2.73-2.72 (m, 1H), 2.69-2.68 (m, 1H), 2.67-2.52 (m, 1H), 2.24-2.15 (m, 1H), 1.86-1.76 (m, 4H), 1.75-1.73 (m, 2H), 1.70-1.66 (m, 1H), 1.65-1.60 (m, 1H), 1.40-1.31 (m, 2H), 1.29-1.21 (m, 1H), 0.93-0.90 (m, 6H), 0.77 (t, J=7.6 Hz, 3H).

MS obsd. (ESI⁺) [(M+H)⁺]: 592.4.

### Example 153

### N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide

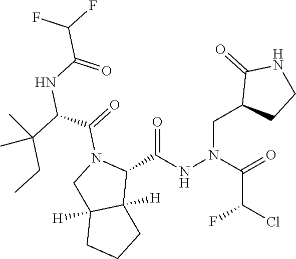 [SMILES: CCC(C)(C)[C@H](NC(=O)C(F)F)C(=O)N1C[C@@H]2CCC[C@@H]2[C@H]1C(=O)NN(C[C@@H]1CCNC1=O)C(=O)[C@@H](F)Cl]

The title compound was prepared in analogy to the procedure described for the preparation of Example 1, by using benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), (2S)-2-chloro-2-fluoro-acetyl chloride (Intermediate 28) instead of (2R)-2-chloro-2-fluoro-acetyl chloride (Intermediate 10), and (2,2-difluoroacetyl) 2,2-difluoroacetate instead of TFAA to afford N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide (Example 153) as a white solid.

¹H NMR (400 MHz, DMSO-d₆) δ: ppm 11.11-10.88 (m, 1H), 8.91 (d, J=8.8 Hz, 1H), 7.78-7.71 (m, 1H), 7.11-6.57 (m, 1H), 6.42-6.13 (m, 1H), 4.45-4.41 (m, 1H), 4.21-4.13 (m, 1H), 3.95-3.89 (m, 1H), 3.80-3.69 (m, 2H), 3.56-3.52 (m, 1H), 3.21-2.98 (m, 2H), 2.23-2.08 (m, 1H), 2.63-2.61 (m, 1H), 1.84-1.72 (m, 1H), 1.66-1.48 (m, 2H), 1.36-1.22 (m, 2H), 1.07-1.02 (m, 3H), 0.94-0.85 (m, 9H), 0.77 (t, J=7.2 Hz, 3H).

MS obsd. (ESI⁺) [(M+H)⁺]: 566.3.

### Example 154

### N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-butyl]-1-fluoro-cyclopropanecarboxamide

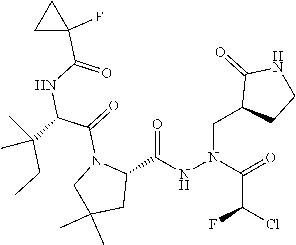 [SMILES: CCC(C)(C)[C@H](NC(=O)C1(F)CC1)C(=O)N1CC(C)(C)C[C@H]1C(=O)NN(C[C@@H]1CCNC1=O)C(=O)[C@H](F)Cl]

The title compound was prepared in analogy to the procedure described for the preparation of Example 1, by using benzyl (2S)-1-[(2S)-2-amino-3,3-dimethyl-pentanoyl]-4,4-dimethyl-pyrrolidine-2-carboxylate; hydrochloride (Intermediate 35) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid and T₃P instead of TFAA to afford N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-butyl]-1-fluoro-cyclopropanecarboxamide (Example 154) as a white solid.

¹H NMR (400 MHz, DMSO-d₆) δ: ppm 11.02 (s, 1H), 7.85-7.63 (m, 1H), 7.48-7.22 (m, 1H), 7.12-6.81 (m, 1H), 4.53 (d, J=8.8 Hz, 1H), 4.32 (dd, J=9.6, 7.6 Hz, 1H), 3.87-3.59 (m, 2H), 3.42 (dd, J=14, 4 Hz, 1H), 3.35-3.32 (m, 1H), 3.23-3.09 (m, 2H), 2.44-2.37 (m, 1H), 2.20-2.04 (m, 1H), 2.01-1.89 (m, 1H), 1.81-1.56 (m, 2H), 1.45-1.16 (m, 6H), 1.14 (s, 3H), 1.05-0.86 (m, 9H), 0.79 (t, J=7.6 Hz, 3H).

MS obsd. (ESI⁺) [(M+H)⁺]: 562.4.

### Example 155a and 155b

### N-[(1S)-1-[(2S)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide and N-[(1S)-1-[(2R)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide

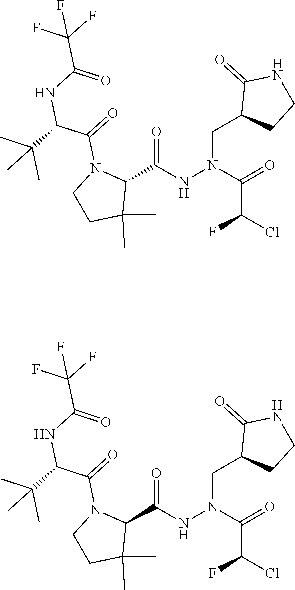 [SMILES: CC(C)(C)[C@H](NC(=O)C(F)(F)F)C(=O)N1CCC(C)(C)[C@H]1C(=O)NN(C[C@@H]1CCNC1=O)C(=O)[C@H](F)Cl ; CC(C)(C)[C@H](NC(=O)C(F)(F)F)C(=O)N1CCC(C)(C)[C@@H]1C(=O)NN(C[C@@H]1CCNC1=O)C(=O)[C@H](F)Cl]

The title compound was prepared in analogy to the procedure described for the preparation of Example 1, by using benzyl 1-[(2S)-2-amino-3,3-dimethyl-butanoyl]-3,3-dimethyl-pyrrolidine-2-carboxylate; 2,2,2-trifluoroacetic acid (Intermediate 36) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1) to afford a mixture of N-[(1S)-1-[(2S)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide and N-[(1S)-1-[(2R)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide. The mixture was split by prep-SFC (DAICEL CHIRALPAK IC (250 mm*30 mm, 10 um); Condition 0.1% NH3H2O IPA Begin B 40; End B 40 Gradient Time(min) 3; 100% B Hold Time(min) FlowRate(ml/min) 120; Injections 25) to afford Example 155a (31.7 mg, retention time=1.45 min) and Example 155b (6.5 mg, retention time=2.94 min) as white solids.

### Example 155a

¹H NMR (400 MHz, DMSO-d₆) δ: ppm 11.15 (s, 1H), 9.48 (d, J=8.4 Hz, 1H), 7.81 (s, 1H), 6.94 (d, J=50.4 Hz, 1H), 4.58 (d, J=8.4 Hz, 1H), 4.06-3.97 (m, 2H), 3.87-3.78 (m, 1H), 3.75-3.68 (m, 1H), 3.25-3.15 (m, 2H), 3.04 (dd, J=13.6 Hz, 3.6 Hz, 1H), 2.60-2.55 (m, 1H), 2.23-2.15 (m, 1H), 1.90-1.83 (m, 1H), 1.81-1.75 (m, 1H), 1.72-1.64 (m, 1H), 1.16-1.09 (m, 3H), 1.02-0.96 (m, 12H).

MS obsd. (ESI⁺) [(M+H)⁺]: 558.3.

### Example 155b

¹H NMR (400 MHz, DMSO-d₆) δ: ppm 11.00-10.52 (m, 1H), 9.25-9.07 (m, 1H), 7.83-7.71 (m, 1H), 7.29-6.52 (m, 1H), 4.78-4.63 (m, 1H), 4.00-3.80 (m, 2H), 3.76-3.51 (m, 2H), 3.30-3.06 (m, 3H), 2.66-2.62 (m, 1H), 2.32-2.14 (m, 1H), 2.08-1.94 (m, 1H), 1.80-1.45 (m, 2H), 1.31-1.14 (m, 3H), 1.10-0.93 (m, 12H).

MS obsd. (ESI⁺) [(M+H)⁺]: 558.2.

### Example 156

### N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide

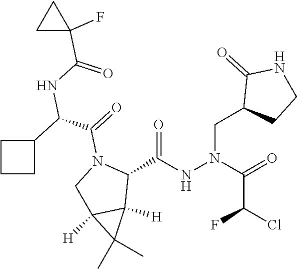 [SMILES: CC1(C)[C@@H]2[C@@H](C(=O)NN(C[C@@H]3CCNC3=O)C(=O)[C@H](F)Cl)N(C(=O)[C@@H](NC(=O)C3(F)CC3)C3CCC3)C[C@@H]21]

The title compound was prepared in analogy to the procedure described for the preparation of Example 1, by using (1R,2S,5S)-3-[(2S)-2-amino-2-cyclobutyl-acetyl]-6,6-dimethyl-3-azabicyclo[3.1.0]hexane-2-carboxylate; 2,2,2-trifluoroacetic acid (Intermediate 37) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid and T₃P instead of TFAA to afford N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide (Example 156) as a white solid.

¹H NMR (400 MHz, DMSO-d₆) δ: ppm 11.08 (s, 1H), 8.33 (d, J=6.8 Hz, 1H), 7.75 (s, 1H), 6.78 (d, J=50.4 Hz, 1H), 4.43 (t, J=8.0 Hz, 1H), 4.14 (s, 1H), 3.92-3.75 (m, 3H), 3.42 (dd, J=14.0 Hz, 4.4 Hz, 1H), 3.22-3.10 (m, 2H), 2.80-2.71 (m, 1H), 2.63-2.53 (m, 2H), 2.20-2.10 (m, 1H), 1.98-1.90 (m, 1H), 1.86-1.71 (m, 5H), 1.66-1.60 (m, 1H), 1.49 (d, J=8.0 Hz, 1H), 1.30-1.25 (m, 2H), 1.21-1.09 (m, 2H), 1.04 (s, 3H), 0.91 (s, 3H).

MS obsd. (ESI⁺) [(M+H)⁺]: 558.2.

### Example 157

### N-[(1S)-2-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide

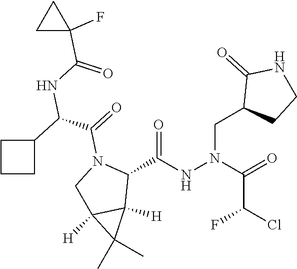 [SMILES: CC1(C)[C@@H]2[C@@H](C(=O)NN(C[C@@H]3CCNC3=O)C(=O)[C@@H](F)Cl)N(C(=O)[C@@H](NC(=O)C3(F)CC3)C3CCC3)C[C@@H]21]

The title compound was prepared in analogy to the procedure described for the preparation of Example 1, by using (1R,2S,5S)-3-[(2S)-2-amino-2-cyclobutyl-acetyl]-6,6-dimethyl-3-azabicyclo[3.1.0]hexane-2-carboxylate; 2,2,2-trifluoroacetic acid (Intermediate 37) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), (2S)-2-chloro-2-fluoro-acetyl chloride (Intermediate 28) instead of (2R)-2-chloro-2-fluoro-acetyl chloride (Intermediate 10), and 1-fluorocyclopropane-1-carboxylic acid and T₃P instead of TFAA to afford N-[(1S)-2-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide (Example 157) as a white solid.

¹H NMR (400 MHz, DMSO-d₆) δ: ppm 10.91-10.72 (m, 1H), 8.32 (s, 1H), 7.73-7.71 (m, 1H), 7.07-6.56 (m, 1H), 4.44 (t, J=8.0 Hz, 1H), 4.18-4.09 (m, 1H), 3.92-3.79 (m, 2H), 3.60 (d, J=7.2 Hz, 1H), 3.23-3.04 (m, 2H), 2.82-2.70 (m, 1H), 2.64-2.56 (m, 1H), 2.20-2.10 (m, 1H), 1.98-1.69 (m, 8H), 1.63-1.44 (m, 2H), 1.33-1.23 (m, 2H), 1.20-1.09 (m, 2H), 1.04 (s, 3H), 0.89 (s, 3H).

MS obsd. (ESI⁺) [(M+H)⁺]: 558.4.

### Example 158

### (1S)—N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide

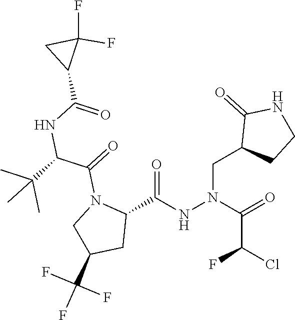 [SMILES: CC(C)(C)[C@H](NC(=O)[C@@H]1CC1(F)F)C(=O)N1C[C@H](C(F)(F)F)C[C@H]1C(=O)NN(C[C@@H]1CCNC1=O)C(=O)[C@H](F)Cl]

The title compound was prepared in analogy to the procedure described for the preparation of Example 1, by using benzyl (2S,4R)-1-[(2S)-2-amino-3,3-dimethyl-butanoyl]-4-(trifluoromethyl)pyrrolidine-2-carboxylate (Intermediate 15) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 2,2-difluorocyclopropanecarboxylic acid and T₃P instead of TFAA to afford (1S)—N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide (Example 158) as a white solid.

¹H NMR (400 MHz, DMSO-d₆) δ: ppm 11.05-10.76 (m, 1H), 8.51 (d, J=8.0 Hz, 1H), 7.70 (s, 1H), 7.18-6.59 (m, 1H), 4.51 (d, J=8.8 Hz, 1H), 4.47-4.25 (m, 1H), 4.05-3.87 (m, 2H), 3.78-3.64 (m, 1H), 3.55-3.45 (m, 1H), 3.20-2.98 (m, 2H), 2.89-2.74 (m, 1H), 2.46-2.35 (m, 1H), 2.24-2.12 (m, 1H), 1.88-1.74 (m, 3H), 1.30-1.17 (m, 2H), 1.02-0.91 (s, 9H), 0.88-0.79 (m, 1H).

MS obsd. (ESI⁺) [(M+H)⁺]: 606.2.

### Example 159

### (1S)—N-[(1S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide

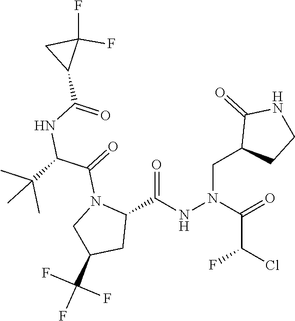 [SMILES: CC(C)(C)[C@H](NC(=O)[C@@H]1CC1(F)F)C(=O)N1C[C@H](C(F)(F)F)C[C@H]1C(=O)NN(C[C@@H]1CCNC1=O)C(=O)[C@@H](F)Cl]

The title compound was prepared in analogy to the procedure described for the preparation of Example 1, by using benzyl (2S,4R)-1-[(2S)-2-amino-3,3-dimethyl-butanoyl]-4-(trifluoromethyl)pyrrolidine-2-carboxylate (Intermediate 15) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), (2S)-2-chloro-2-fluoro-acetyl chloride (Intermediate 28) instead of (2R)-2-chloro-2-fluoro-acetyl chloride (Intermediate 10), and (1S)-2,2-difluorocyclopropanecarboxylic acid and T₃P instead of TFAA to afford (1S)—N-[(1S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide (Example 159) as a white solid.

¹H NMR (400 MHz, DMSO-d₆) δ: ppm 11.10 (br. s, 1H), 8.53 (d, J=8.4 Hz, 1H), 7.72 (s, 1H), 6.96 (d, J=50.4 Hz, 1H), 4.50 (d, J=8.4 Hz, 1H), 4.38-4.30 (m, 1H), 4.07-3.98 (m, 2H), 3.70 (dd, J=14.0 Hz, 8.4 Hz, 1H), 3.53 (dd, J=14.0 Hz, 4.0 Hz, 1H), 3.47-3.39 (m, 1H), 3.19-3.10 (m, 2H), 2.94-2.83 (m, 1H), 2.60-2.55 (m, 1H), 2.40-2.33 (m, 1H), 2.27-2.18 (m, 1H), 2.16-2.07 (m, 1H), 1.93-1.75 (m, 3H), 0.96 (s, 9H).

MS obsd. (ESI⁺) [(M+H)⁺]: 606.2.

### Example 160

### N-[(1S,2S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide

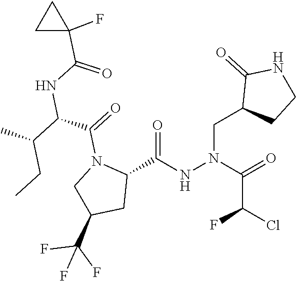 [SMILES: CC[C@H](C)[C@H](NC(=O)C1(F)CC1)C(=O)N1C[C@H](C(F)(F)F)C[C@H]1C(=O)NN(C[C@@H]1CCNC1=O)C(=O)[C@H](F)Cl]

The title compound was prepared in analogy to the procedure described for the preparation of Example 1, by using benzyl (2S,4R)-1-[(2S,3S)-2-amino-3-methyl-pentanoyl]-4-(trifluoromethyl)pyrrolidine-2-carboxylate; 2,2,2-trifluoroacetaldehyde (Intermediate 38) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid and T₃P instead of TFAA to afford N-[(1S,2S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide (Example 160) as a white solid.

¹H NMR (400 MHz, DMSO-d₆) δ: ppm 11.07 (s, 1H), 8.60-8.43 (m, 1H), 7.72 (s, 1H), 6.92 (d, J=50.4 Hz, 1H), 4.43-4.28 (m, 2H), 4.24-4.09 (m, 1H), 4.00-3.90 (m, 1H), 3.76-3.65 (m, 1H), 3.53 (dd, J=14.4 Hz, 4.0 Hz, 1H), 3.43-3.35 (m, 1H), 3.20-3.07 (m, 2H), 2.60-2.53 (m, 1H), 2.40-2.34 (m, 1H), 2.28-2.19 (m, 1H), 2.16-2.03 (m, 1H), 2.02-1.91 (m, 1H), 1.86-1.73 (m, 1H), 1.52-1.45 (m, 1H), 1.35-1.25 (m, 2H), 1.24-1.07 (m, 3H), 0.87-0.79 (m, 6H).

MS obsd. (ESI⁺) [(M+H)⁺]: 588.3.

### Example 161

### N-[(1S,2S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide

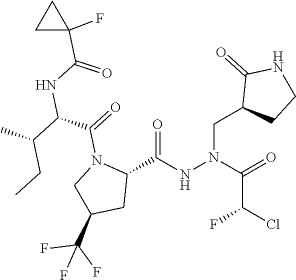 [SMILES: CC[C@H](C)[C@H](NC(=O)C1(F)CC1)C(=O)N1C[C@H](C(F)(F)F)C[C@H]1C(=O)NN(C[C@@H]1CCNC1=O)C(=O)[C@@H](F)Cl]

The title compound was prepared in analogy to the procedure described for the preparation of Example 1, by using benzyl (2S,4R)-1-[(2S,3S)-2-amino-3-methyl-pentanoyl]-4-(trifluoromethyl)pyrrolidine-2-carboxylate; 2,2,2-trifluoroacetaldehyde (Intermediate 38) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), (2S)-2-chloro-2-fluoro-acetyl chloride (Intermediate 28) instead of (2R)-2-chloro-2-fluoro-acetyl chloride (Intermediate 10), and 1-fluorocyclopropane-1-carboxylic acid and T₃P instead of TFAA to afford N-[(1S,2S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide (Example 161) as a white solid.

¹H NMR (400 MHz, DMSO-d₆) δ: ppm 11.00-10.80 (m, 1H), 8.60-8.47 (m, 1H), 7.72 (s, 1H), 7.22-6.63 (m, 1H), 4.50-4.27 (m, 2H), 4.17-4.02 (m, 1H), 4.00-3.91 (m, 1H), 3.72-3.57 (m, 2H), 3.52-3.36 (m, 1H), 3.18-3.01 (m, 2H), 2.53-2.51 (m, 1H), 2.40-2.34 (m, 1H), 2.27-2.07 (m, 2H), 2.03-1.90 (m, 1H), 1.83-1.73 (m, 1H), 1.57-1.45 (m, 1H), 1.34-1.25 (m, 2H), 1.22-1.08 (m, 3H), 0.86 (d, J=6.8 Hz, 3H), 0.81 (t, J=7.2 Hz, 3H).

MS obsd. (ESI⁺) [(M+H)⁺]: 588.4.

### Example 162

### N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-1-fluoro-cyclopropanecarboxamide

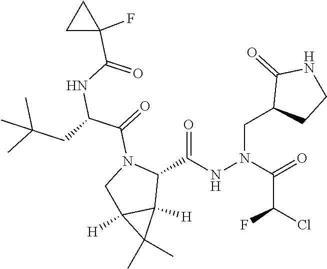 [SMILES: CC(C)(C)C[C@H](NC(=O)C1(F)CC1)C(=O)N1C[C@H]2[C@@H]([C@H]1C(=O)NN(C[C@@H]1CCNC1=O)C(=O)[C@H](F)Cl)C2(C)C]

The title compound was prepared in analogy to the procedure described for the preparation of Example 1, by using benzyl (1R,2S,5S)-3-[(2S)-2-amino-4,4-dimethyl-pentanoyl]-6,6-dimethyl-3-azabicyclo[3.1.0]hexane-2-carboxylate; 2,2,2-trifluoroacetic acid (Intermidiate 42) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and 1-fluorocyclopropane-1-carboxylic acid and T₃P instead of TFAA to afford N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-1-fluoro-cyclopropanecarboxamide (Example 162) as a white solid.

¹H NMR (400 MHz, DMSO-d₆) δ: ppm 11.09 (s, 1H), 8.56-8.46 (m, 1H), 7.77 (s, 1H), 6.78 (d, J=50.8 Hz, 1H), 4.56-4.51 (m, 1H), 4.14 (s, 1H), 3.86-3.79 (m, 3H), 3.52-3.39 (m, 2H), 3.21-3.13 (m, 2H), 2.63-2.56 (m, 1H), 2.23-2.12 (m, 1H), 1.80-1.69 (m, 2H), 1.66-1.62 (m, 1H), 1.56-1.50 (m, 2H), 1.32-1.24 (m, 2H), 1.19-1.06 (m, 2H), 1.05 (s, 3H), 0.95-0.90 (m, 11H).

MS obsd. (ESI⁺) [(M+H)⁺]: 574.3.

### Example 163a and 163b

### N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide and N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide

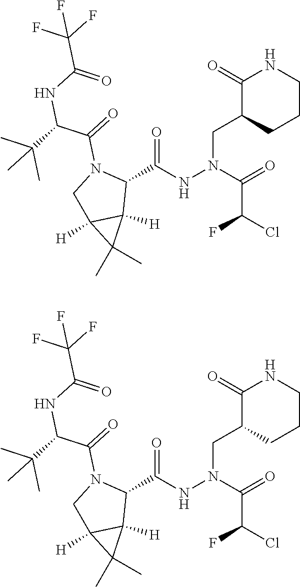 [SMILES: CC(C)(C)[C@H](NC(=O)C(F)(F)F)C(=O)N1C[C@H]2[C@@H]([C@H]1C(=O)NN(C[C@@H]1CCCNC1=O)C(=O)[C@H](F)Cl)C2(C)C ; CC(C)(C)[C@H](NC(=O)C(F)(F)F)C(=O)N1C[C@H]2[C@@H]([C@H]1C(=O)NN(C[C@H]1CCCNC1=O)C(=O)[C@H](F)Cl)C2(C)C]

### Step 1: Preparation of benzyl (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylate

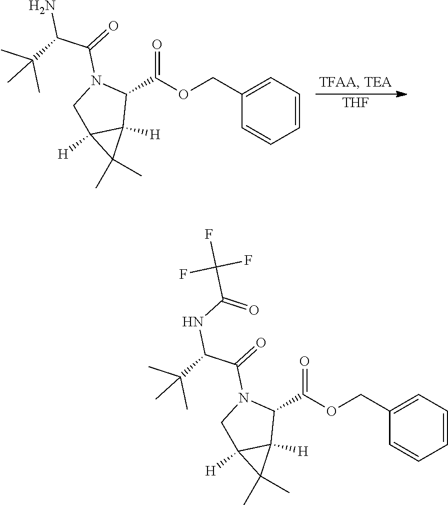 [SMILES: CC(C)(C)[C@H](N)C(=O)N1C[C@H]2[C@@H]([C@H]1C(=O)OCc1ccccc1)C2(C)C ; CC(C)(C)[C@H](NC(=O)C(F)(F)F)C(=O)N1C[C@H]2[C@@H]([C@H]1C(=O)OCc1ccccc1)C2(C)C ; "THF"]

To a solution of benzyl (1R,2S,5S)-3-[(2S)-2-amino-3,3-dimethyl-butanoyl]-6,6-dimethyl-3-azabicyclo[3.1.0]hexane-2-carboxylate (970 mg, 2.71 mmol, Intermediate 1) in THF (20 mL) was added TEA (601 mg, 5.95 mmol) and TFAA (1131 mg, 5.41 mmol) at 0° C. The reaction mixture was stirred at 25° C. for 1 h. The reaction was quenched with water (50 mL) and extracted with EtOAc (80 mL×2). The organic layers were washed with brine (50 mL), dried over anhydrous Na₂SO₄, filtered and concentrated in vacuum. The residue was purified by silica gel column eluted with PE to EtOAc/PE=10/1 to afford benzyl (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylate (1.2 g) as colorless oil.

MS obsd. (ESI⁺) [M+H]⁺: 455.3.

### Step 2: Preparation of (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid

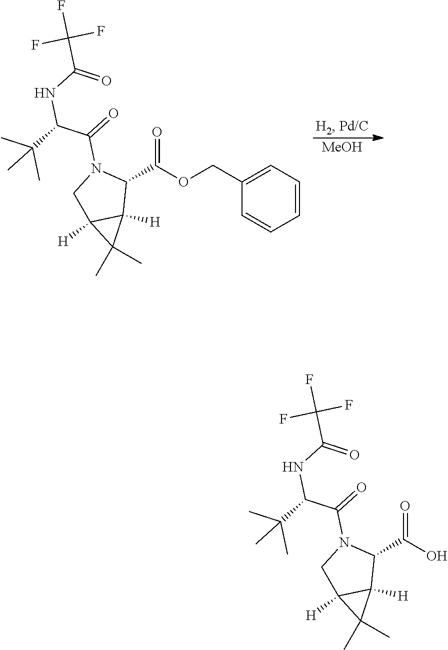 [SMILES: "MeOH" ; CC(C)(C)[C@H](NC(=O)C(F)(F)F)C(=O)N1C[C@H]2[C@@H]([C@H]1C(=O)OCc1ccccc1)C2(C)C ; CC(C)(C)[C@H](NC(=O)C(F)(F)F)C(=O)N1C[C@H]2[C@@H]([C@H]1C(=O)O)C2(C)C]

To a solution of benzyl (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylate (1.2 g, 2.64 mmol) in MeOH (20 mL) was added Pd/C (200 mg, 10% purity). The mixture was degassed under vacuum and purged H₂ for 3 times. The reaction mixture was stirred at 25° C. for 1 h under H₂ balloon. The mixture was filtered through a celite pad and the pad was washed with MeOH (10 mL×3). The filtrate was concentrated in vacuum to afford (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (960 mg) as a white solid.

MS obsd. (ESI⁺) [M+H]⁺: 365.2.

### Step 3: Preparation of tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3R)-2-oxo-3-piperidyl]methyl]carbamate and tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxo-3-piperidyl]methyl]carbamate

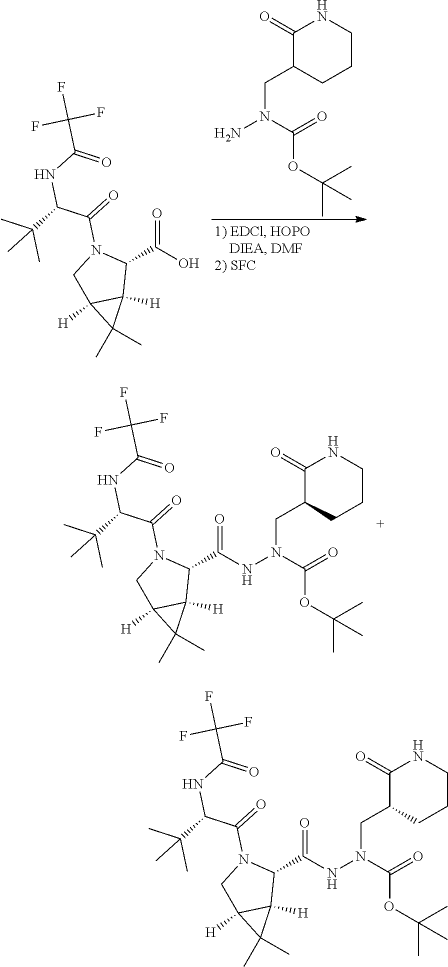

To a solution of (1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carboxylic acid (380 mg, 1.04 mmol) in DMF (4 mL) was added EDCI (280 mg, 1.46 mmol), HOPO (162 mg, 1.46 mmol), DIPEA (674 mg, 5.21 mmol) and tert-butyl N-amino-N-[(2-oxo-3-piperidyl)methyl]carbamate (254 mg, 1.04 mmol, from step 5 of Intermediate 39) at 0° C. Then the mixture was stirred at 20° C. for 12 h. The resulting mixture was poured into EtOAc (150 mL) and washed with 1 N HCl (20 mL), 5% aqueous solution of K₂CO₃ (50 mL) and brine (50 mL). The organic phase was dried over anhydrous Na₂SO₄, filtered and concentrated in vacuum. The residue was purified by reverse flash chromatography (condition: 120 g Flash Column Welch Ultimate XB_C18 20-40 μm; 120 A, water (0.1% HCl)-ACN, 0˜50%, 70 mL/min) to afford a mixture of tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3R)-2-oxo-3-piperidyl]methyl]carbamate and tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxo-3-piperidyl]methyl]carbamate (295 mg) as light yellow solids. The mixture was split by SFC (instrument: ACSWH-PREP-SFC-C, column: DAICEL CHIRALPAK IC (250 mm*30 mm, 10 um), mobile phase: 40% Neu-IPA in supercritical CO₂, flow rate: 75 mL/min, cycle time: 3.2 min, back pressure: 100 bar to keep the CO₂ in suspercritical flow, UV: 220 nm) to afford Peak 1 (106 mg, retention time=1.25 min) and Peak 2 (172 mg, retention time=2.76 min) as light yellow solids.

### Peak 1

MS obsd. (ESI⁺) [(M+H)⁺]: 590.4.

### Peak 2

MS obsd. (ESI⁺) [(M+H)⁺]: 590.4.

### Step 4: Preparation of N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide and N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide

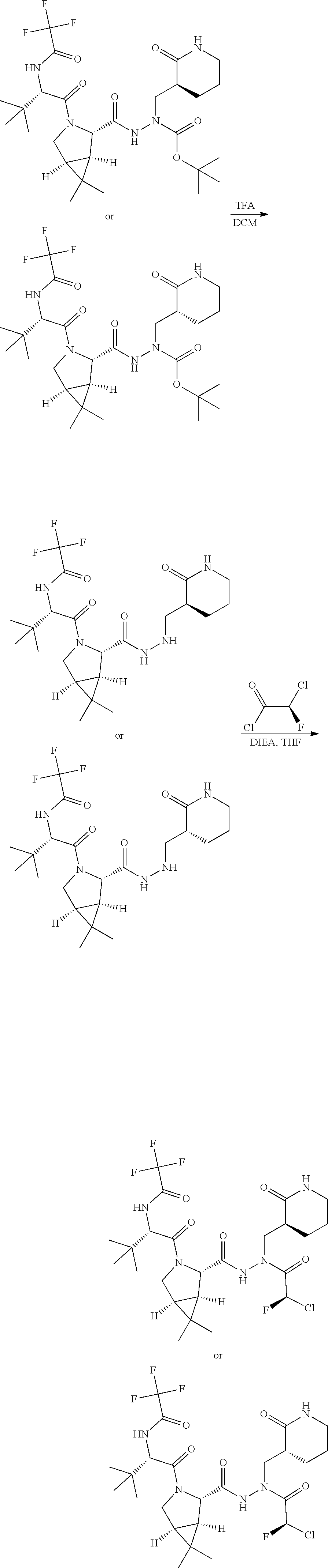

These compounds were prepared in analogy to the procedure described for the preparation of Example 1, by using tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3R)-2-oxo-3-piperidyl]methyl]carbamate or tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxo-3-piperidyl]methyl]carbamate instead of tert-butyl N-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]-N-[[(3S)-2-oxopyrrolidin-3-yl]methyl]carbamate to afford Example 163a or Example 163b respectively as a white solid.

### Example 163a

¹H NMR (400 MHz, DMSO-d₆) δ: ppm 11.13 (s, 1H), 9.52 (d, J=7.6 Hz, 1H), 7.54 (s, 1H), 6.82 (d, J=50.8 Hz, 1H), 4.42-4.38 (m, 1H), 4.18 (s, 1H), 3.98-3.86 (m, 2H), 3.72 (d, J=10.4 Hz, 1H), 3.63 (dd, J=14.0 Hz, 8.4 Hz, 1H), 3.16-3.09 (m, 2H), 2.42-2.34 (m, 1H), 1.94-1.87 (m, 1H), 1.80-1.74 (m, 1H), 1.63-1.60 (m, 1H), 1.59-1.52 (m, 3H), 1.05 (s, 3H), 0.99 (s, 9H), 0.91 (s, 3H).

MS obsd. (ESI⁺) [(M+H)⁺]: 584.3.

### Example 163b

¹H NMR (400 MHz, DMSO-d₆) δ: ppm 11.21 (s, 1H), 9.55 (d, J=8.0 Hz, 1H), 7.59 (s, 1H), 6.87 (d, J=50.8 Hz, 1H), 4.44 (d, J=8.0 Hz, 1H), 4.19 (s, 1H), 4.09-3.95 (m, 2H), 3.76 (d, J=10.8 Hz, 1H), 3.48 (dd, J=14.0 Hz, 4.0 Hz, 1H), 3.16-3.11 (m, 2H), 2.50-2.45 (m, 1H), 1.88-1.77 (m, 2H), 1.71-1.67 (m, 1H), 1.66-1.55 (m, 1H), 1.53-1.47 (m, 2H), 1.08 (s, 3H), 1.01 (s, 9H), 0.93 (s, 3H).

MS obsd. (ESI⁺) [(M+H)⁺]: 584.3.

### Example 164a and 164b

### N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide and N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide

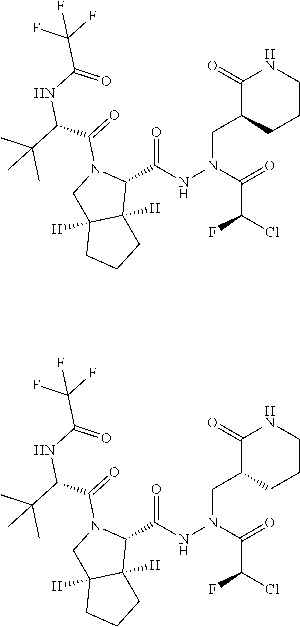 [SMILES: CC(C)(C)[C@H](NC(=O)C(F)(F)F)C(=O)N1C[C@@H]2CCC[C@@H]2[C@H]1C(=O)NN(C[C@@H]1CCCNC1=O)C(=O)[C@H](F)Cl ; CC(C)(C)[C@H](NC(=O)C(F)(F)F)C(=O)N1C[C@@H]2CCC[C@@H]2[C@H]1C(=O)NN(C[C@H]1CCCNC1=O)C(=O)[C@H](F)Cl]

The title compound was prepared in analogy to the procedure described for the preparation of Example 1, by using benzyl (3S,3aS,6aR)-2-[(2S)-2-amino-3,3-dimethyl-butanoyl]-3,3a,4,5,6,6a-hexahydro-1H-cyclopenta[c]pyrrole-3-carboxylate (Intermediate 3) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), and tert-butyl N-amino-N-[(2-oxo-3-piperidyl)methyl]carbamate (racemic, from Intermediate 39) instead of tert-butyl N-amino-N-[[(3S)-2-oxopyrrolidin-3-yl]methyl]carbamate (Intermediate 9) to afford a mixture of N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide and N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide. The mixture was split by prep-SFC (Instrument:Waters 80Q; Mobile Phase: 40% EtOH (Neu) in Supercritical CO₂; Flow Rate: 70 g/min; Cycle Time: 2.8 min, total time: 25 min; Single injection volume: 3.0 mL; Back Pressure: 100 bar to keep the CO₂ in Supercritical flow) to afford Example 164a (retention time=1.19 min) and Example 164b (retention time=2.40 min) as white solids.

### Example 164a

¹H NMR (400 MHz, DMSO-d₆) δ: ppm 11.10 (s, 1H), 9.46 (d, J=8.4 Hz, 1H), 7.57-7.49 (m, 1H), 6.90 (d, J=50.4 Hz, 1H), 4.53 (d, J=8.0 Hz, 1H), 4.05-3.85 (m, 3H), 3.68 (dd, J=10.4 Hz, 2.8 Hz, 1H), 3.42-3.39 (m, 1H), 3.15-3.08 (m, 2H), 2.82-2.70 (m, 1H), 2.58-2.53 (m, 1H), 2.46-2.35 (m, 1H), 1.92-1.71 (m, 6H), 1.69-1.54 (m, 2H), 1.52-1.33 (m, 2H), 0.99 (s, 9H).

MS obsd. (ESI⁺) [(M+H)⁺]: 584.3.

### Example 164b

¹H NMR (400 MHz, DMSO-d₆) δ: ppm 11.01 (s, 1H), 9.44 (d, J=7.6 Hz, 1H), 7.49 (s, 1H), 6.94 (d, J=50.4 Hz, 1H), 4.51 (d, J=8.0 Hz, 1H), 4.01 (d, J=8.4 Hz, 1H), 3.96-3.85 (m, 2H), 3.70 (dd, J=10.4 Hz, 2.4 Hz, 1H), 3.57 (dd, J=13.6 Hz, 8.0 Hz, 1H), 3.20-3.02 (m, 2H), 2.79-2.69 (m, 1H), 2.64-2.56 (m, 1H), 2.35-2.25 (m, 1H), 1.95-1.70 (m, 6H), 1.64-1.45 (m, 3H), 1.43-1.31 (m, 1H), 0.99 (s, 9H).

MS obsd. (ESI⁺) [(M+H)⁺]: 584.3.

### Example 165a and 165b

### N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide and N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide

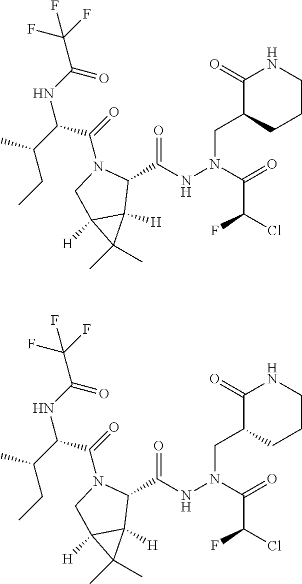 [SMILES: CC[C@H](C)[C@H](NC(=O)C(F)(F)F)C(=O)N1C[C@H]2[C@@H]([C@H]1C(=O)NN(C[C@@H]1CCCNC1=O)C(=O)[C@H](F)Cl)C2(C)C ; CC[C@H](C)[C@H](NC(=O)C(F)(F)F)C(=O)N1C[C@H]2[C@@H]([C@H]1C(=O)NN(C[C@H]1CCCNC1=O)C(=O)[C@H](F)Cl)C2(C)C]

### Step 1: Preparation of methyl (1R,2S,5S)-3-[(2S,3S)-2-(tert-butoxycarbonylamino)-3-methyl-pentanoyl]-6,6-dimethyl-3-azabicyclo[3.1.0]hexane-2-carboxylate

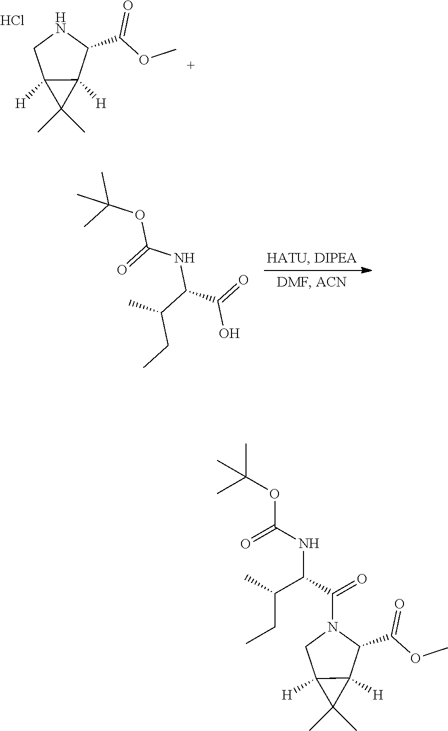

To a solution of Boc-Ile-OH (96.0 g, 415.06 mmol) in DMF (192 mL) and MeCN (1536 mL) were added methyl (1R,2S,5S)-6,6-dimethyl-3-azabicyclo[3.1.0]hexane-2-carboxylate; hydrochloride (89.64 g, 435.82 mmol) and HATU (173.6 g, 456.57 mmol) at 0° C. under N₂ to give a colorless solution, followed by drop-wise addition of DIPEA (216.88 mL, 1245.19 mmol) at 0° C. under N₂ to give a light yellow solution. The reaction was then allowed to warm to 20° C. and was stirred for 16 h under N₂ atmosphere and concentrated in vacuum. The residue was added to ethyl acetate (2400 mL) and water (3600 mL). The resulting suspension was filtered and the filter cake was washed with ethyl acetate (500 mL), and the filter cake was dried in vacuum to give methyl (1R,2S,5S)-3-[(2S,3S)-2-(tert-butoxycarbonylamino)-3-methyl-pentanoyl]-6,6-dimethyl-3-azabicyclo[3.1.0]hexane-2-carboxylate (75.0 g) as a white solid.

MS obsd. (ESI⁺) [(M+H)⁺]: 383.2.

### Step 2: Preparation of (1R,2S,5S)-3-[(2S,3S)-2-(tert-butoxycarbonylamino)-3-methyl-pentanoyl]-6,6-dimethyl-3-azabicyclo[3.1.0]hexane-2-carboxylic acid

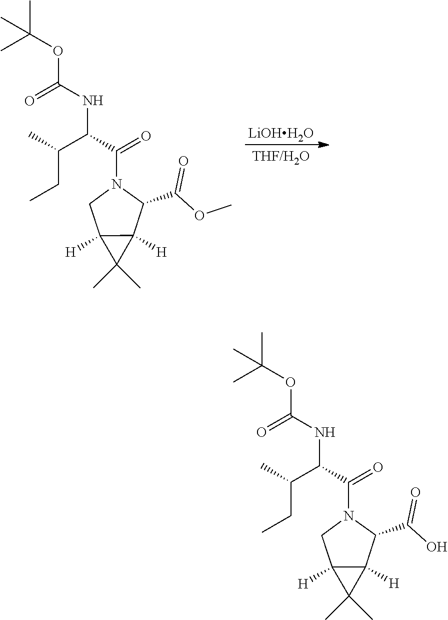 [SMILES: CC[C@H](C)[C@H](NC(=O)OC(C)(C)C)C(=O)N1C[C@H]2[C@@H]([C@H]1C(=O)OC)C2(C)C ; "LiOH·H2O" ; CC[C@H](C)[C@H](NC(=O)OC(C)(C)C)C(=O)N1C[C@H]2[C@@H]([C@H]1C(=O)O)C2(C)C]

To a solution of methyl (1R,2S,5S)-3-[(2S,3S)-2-(tert-butoxycarbonylamino)-3-methyl-pentanoyl]-6,6-dimethyl-3-azabicyclo[3.1.0]hexane-2-carboxylate (50.0 g, 130.72 mmol) in THF (500 mL). The mixture was added a solution of LiOH·H₂O (10.97 g, 261.4 mmol) in water (500 mL). Then the mixture was stirred at 25° C. for 2 h. The reaction mixture was poured into ice/water (300 mL) and acidified with 12N HCl to pH=5. Then the mixture was extracted with EA(500 ml×2). The combined organic phases were washed with brine (100 mL), dried over Na₂SO₄ and concentrated in vacuum to give (1R,2S,5S)-3-[(2S,3S)-2-(tert-butoxycarbonylamino)-3-methyl-pentanoyl]-6,6-dimethyl-3-azabicyclo[3.1.0]hexane-2-carboxylic acid (48.1 g) as yellow oil.

MS obsd. (ESI⁺) [(M+H)⁺]: 369.2.

### Step 3: Preparation of (1R,2S,5S)-3-[(2S,3S)-2-amino-3-methyl-pentanoyl]-6,6-dimethyl-3-azabicyclo[3.1.0]hexane-2-carboxylic acid; hydrochloride

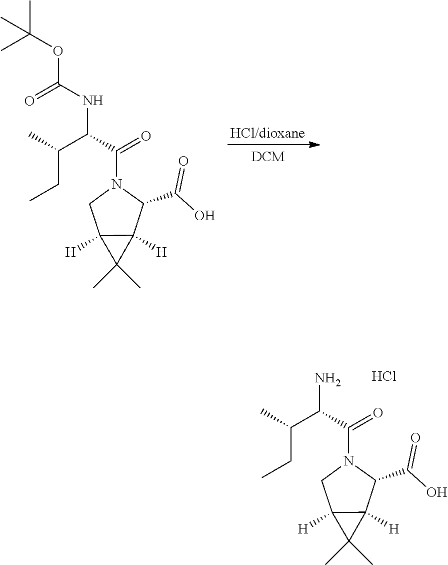 [SMILES: CC[C@H](C)[C@H](NC(=O)OC(C)(C)C)C(=O)N1C[C@H]2[C@@H]([C@H]1C(=O)O)C2(C)C ; Cl ; CC[C@H](C)[C@H](N)C(=O)N1C[C@H]2[C@@H]([C@H]1C(=O)O)C2(C)C]

To a solution of (1R,2S,5S)-3-[(2S,3S)-2-(tert-butoxycarbonylamino)-3-methyl-pentanoyl]-6,6-dimethyl-3-azabicyclo[3.1.0]hexane-2-carboxylic acid (48.1 g, 130.54 mmol) in DCM (500 mL) was added HCl/dioxane (500.0 mL, 2000.0 mmol). The mixture was stirred at 25° C. for 2 h and concentrated in vacuum to afford (1R,2S,5S)-3-[(2S,3S)-2-amino-3-methyl-pentanoyl]-6,6-dimethyl-3-azabicyclo[3.1.0]hexane-2-carboxylic acid; hydrochloride (39.8 g) as a yellow foam.

### Step 4: Preparation of (1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carboxylic acid

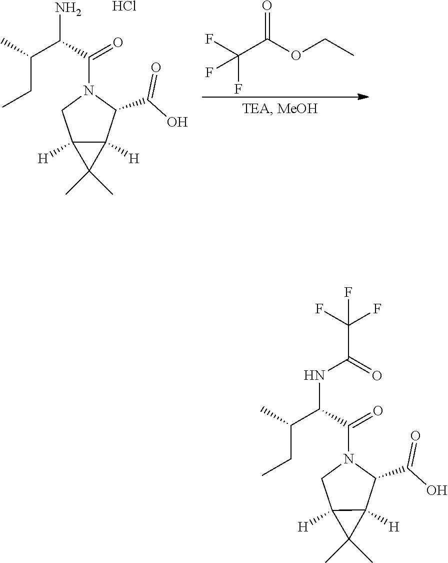

To a solution of (1R,2S,5S)-3-[(2S,3R)-2-amino-3-methyl-pentanoyl]-6,6-dimethyl-3-azabicyclo[3.1.0]hexane-2-carboxylic acid; hydrochloride (39.79 g, 130.54 mmol) in methanol (400 mL) was added TEA (79.26 g, 783.24 mmol) dropwise at 0° C., followed by ethyl trifluoroacetate (55.64 g, 391.62 mmol). Then the mixture was warmed to 50° C. and stirred at 50° C. for 12 h. The mixture was diluted in EtOAc (500 mL), washed with brine (300 ml), dried over Na₂SO₄ and concentrated to give (1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carboxylic acid (45.0 g) as a yellow foam.

MS obsd. (ESI⁺) [(M+H)⁺]: 365.1.

### Step 5: Preparation of tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3S)-2-oxo-3-piperidyl]methyl]carbamate and tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3R)-2-oxo-3-piperidyl]methyl]carbamate

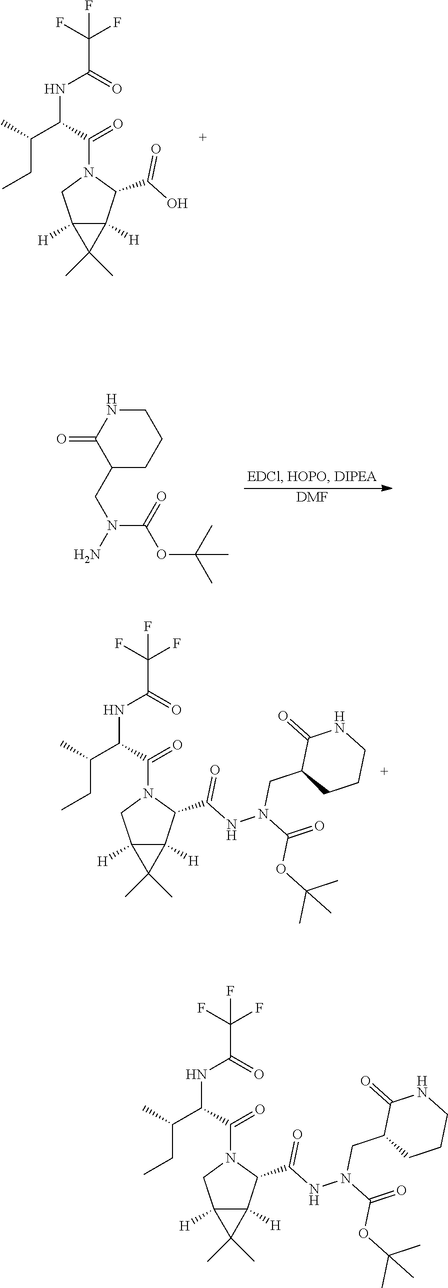

To a solution of (1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carboxylic acid (500 mg, 1.37 mmol) in DMF (10 mL) was added DIPEA (887 mg, 6.86 mmol), EDCI (368 mg, 1.92 mmol), HOPO (213.44 mg, 1.92 mmol) and tert-butyl N-amino-N-[(2-oxo-3-piperidyl)methyl]carbamate (334 mg, 1.37 mmol, racemic) at 0° C. The mixture was stirred at 20° C. for 12 h. The reaction mixture was poured into EtOAc (150 mL) and washed with 1 N HCl (20 mL), 5% aqueous solution of K₂CO₃ (50 mL) and brine (50 mL). The organic phase was dried over Na₂SO₄, filtered and concentrated in vacuum. The residue was purified by reverse flash chromatography (condition: 120 g Flash Column Welch Ultimate XB_C18 20-40 μm; 120 A, water (0.1% FA)-TFA, 0˜49%, 70 mL/min) to afford a mixture of tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3S)-2-oxo-3-piperidyl]methyl]carbamate and tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3R)-2-oxo-3-piperidyl]methyl]carbamate. The mixture was split by prep-SFC (instrument: ACSWH-PREP-SFC-C, column: DAICEL CHIRALPAK IC (250 mm*30 mm, 10 um), mobile phase: 40% Neu-IPA in supercritical CO₂, flow rate: 75 mL/min, cycle time: 3.2 min, back pressure: 100 bar to keep the CO₂ in suspercritical flow, UV: 220 nm) to afford Peak 1 (204 mg, retention time=1.76 min) and Peak 2 (136 mg, retention time=3.77 min) as yellow solids.

### Peak 1

MS obsd. (ESI⁺) [(M+H)⁺]: 590.3.

### Peak 2

MS obsd. (ESI⁺) [(M+H)⁺]: 590.3.

### Step 6: Preparation of N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide and N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide

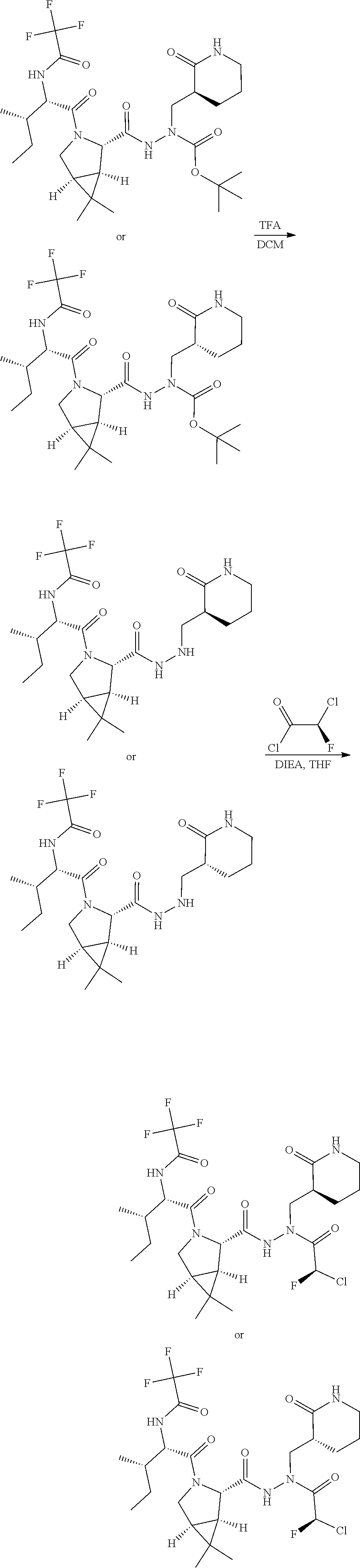

These compounds were prepared in analogy to the procedure described for the preparation of Example 64, by using tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3S)-2-oxo-3-piperidyl]methyl]carbamate or tert-butyl N-[[(1R,2S,5S)-6,6-dimethyl-3-[(2S,3S)-3-methyl-2-[(2,2,2-trifluoroacetyl)amino]pentanoyl]-3-azabicyclo[3.1.0]hexane-2-carbonyl]amino]-N-[[(3R)-2-oxo-3-piperidyl]methyl]carbamate instead of tert-butyl N-[[(1R,2S,5S)-6,6-dimethyl-3-[(2S,3R)-3-methyl-2-[(2,2,2-trifluoroacetyl)amino]pentanoyl]-3-azabicyclo[3.1.0]hexane-2-carbonyl]amino]-N-[[(3S)-2-oxopyrrolidin-3-yl]methyl]carbamate to afford Example 165a and Example 165b respectively as a white solid.

### Example 165a

¹H NMR (400 MHz, DMSO-d₆) δ: ppm 11.19 (s, 1H), 9.99 (d, J=7.6 Hz, 1H), 7.59 (s, 1H), 6.86 (d, J=50.8 Hz, 1H), 4.21-4.17 (m, 1H), 4.19 (s, 1H), 4.08-3.98 (m, 1H), 3.97-3.87 (m, 2H), 3.49 (dd, J=14.0 Hz, 4.0 Hz, 1H), 3.19-3.10 (m, 2H), 2.51-2.43 (m, 1H), 2.01-1.93 (m, 1H), 1.90-1.77 (m, 2H), 1.74-1.68 (m, 1H), 1.67-1.50 (m, 4H), 1.20-1.12 (m, 1H), 1.09 (s, 3H), 0.95 (s, 3H), 0.91 (d, J=6.8 Hz, 3H), 0.85 (t, J=7.2 Hz, 3H).

MS obsd. (ESI⁺) [(M+H)⁺]: 584.2.

### Example 165b

¹H NMR (400 MHz, DMSO-d₆) δ: ppm 11.09 (s, 1H), 9.96-9.90 (m, 1H), 7.52 (s, 1H), 6.80 (d, J=50.4 Hz, 1H), 4.18-4.16 (m, 1H), 4.17 (s, 1H), 3.94-3.81 (m, 3H), 3.63 (dd, J=14.4 Hz, 8.8 Hz, 1H), 3.18-3.12 (m, 2H), 2.42-2.35 (m, 1H), 1.96-1.87 (m, 2H), 1.81-1.72 (m, 1H), 1.68-1.61 (m, 1H), 1.59-1.51 (m, 3H), 1.50-1.42 (m, 1H), 1.15-1.11 (m, 1H), 1.05 (s, 3H), 0.91 (s, 3H), 0.86 (d, J=6.8 Hz, 3H), 0.81 (t, J=7.2 Hz, 3H).

MS obsd. (ESI⁺) [(M+H)⁺]: 584.2.

### Example 166a and 166b

### N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide and N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide

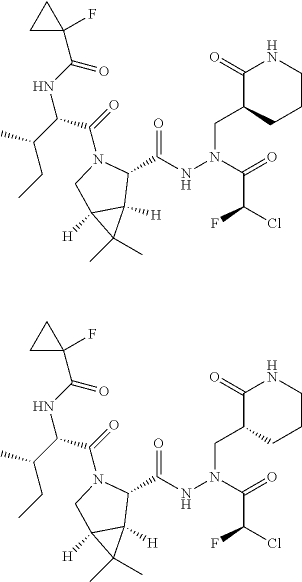 [SMILES: CC[C@H](C)[C@H](NC(=O)C1(F)CC1)C(=O)N1C[C@H]2[C@@H]([C@H]1C(=O)NN(C[C@@H]1CCCNC1=O)C(=O)[C@H](F)Cl)C2(C)C ; CC[C@H](C)[C@H](NC(=O)C1(F)CC1)C(=O)N1C[C@H]2[C@@H]([C@H]1C(=O)NN(C[C@H]1CCCNC1=O)C(=O)[C@H](F)Cl)C2(C)C]

The title compound was prepared in analogy to the procedure described for the preparation of Example 165a and 165b, by using 1-fluorocyclopropane-1-carboxylic acid and T₃P instead of TFAA to afford N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide and N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide as a white solid.

### Example 166a

¹H NMR (400 MHz, DMSO-d₆) δ: ppm 11.44-11.12 (m, 1H), 8.53-8.42 (m, 1H), 7.56-7.47 (m, 1H), 6.87 (d, J=50.8 Hz, 1H), 4.25-4.20 (m, 1H), 4.13 (s, 1H), 4.02-3.94 (m, 2H), 3.87-3.82 (m, 1H), 3.44 (dd, J=14.0 Hz, 4.0 Hz, 1H), 3.14-3.07 (m, 2H), 2.46-2.40 (m, 1H), 2.00-1.91 (m, 1H), 1.84-1.76 (m, 2H), 1.64-1.57 (m, 2H), 1.56-1.42 (m, 3H), 1.31-1.15 (m, 3H), 1.16-1.07 (m, 2H), 1.04 (s, 3H), 0.89 (s, 3H), 0.83-0.77 (m, 6H).

MS obsd. (ESI⁺) [(M+H)⁺]: 574.3.

### Example 166b

¹H NMR (400 MHz, DMSO-d₆) δ: ppm 11.07 (s, 1H), 8.52 (d, J=7.6 Hz, 1H), 7.51 (s, 1H), 6.83 (d, J=50.4 Hz, 1H), 4.21 (dd, J=9.6 Hz, 8.4 Hz, 1H), 4.15 (s, 1H), 3.96-3.82 (m, 3H), 3.63 (dd, J=14.4 Hz, 8.8 Hz, 1H), 3.43-3.36 (m, 1H), 3.15-3.07 (m, 2H), 2.40-2.32 (m, 1H), 2.00-1.90 (m, 2H), 1.81-1.74 (m, 1H), 1.63-1.45 (m, 5H), 1.32-1.15 (m, 3H), 1.11-1.05 (m, 1H), 1.03 (s, 3H), 0.89 (s, 3H), 0.84-0.78 (m, 6H).

MS obsd. (ESI⁺) [(M+H)⁺]: 574.3.

### Example 167a and 167b

### N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide and N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide

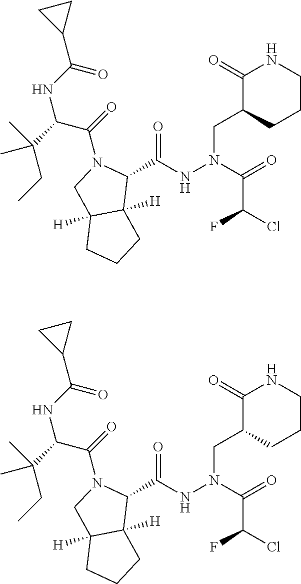 [SMILES: CCC(C)(C)[C@H](NC(=O)C1CC1)C(=O)N1C[C@@H]2CCC[C@@H]2[C@H]1C(=O)NN(C[C@@H]1CCCNC1=O)C(=O)[C@H](F)Cl ; CCC(C)(C)[C@H](NC(=O)C1CC1)C(=O)N1C[C@@H]2CCC[C@@H]2[C@H]1C(=O)NN(C[C@H]1CCCNC1=O)C(=O)[C@H](F)Cl]

The title compound was prepared in analogy to the procedure described for the preparation of Example 1, by using benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[(2-oxo-3-piperidyl)methyl]carbamate (racemic, from Intermediate 39) instead of tert-butyl N-amino-N-[[(3S)-2-oxopyrrolidin-3-yl]methyl]carbamate (Intermediate 9), and cyclopropanecarbonyl chloride instead of TFAA to afford a mixture of N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide and N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide. The mixture was split by prep-SFC (Instrument:Waters 80Q; Mobile Phase: 35% EtOH (Neu) in Supercritical CO₂; Flow Rate: 60 g/min; Cycle Time: 3.6 min, total time: 25 min; Single injection volume: 3.5 mL; Back Pressure:100 bar to keep the CO₂ in Supercritical flow.) to afford Example 167a (retention time=1.26 min) and Example 167b (retention time=2.61 min) as a white solid.

### Example 167a

¹H NMR (400 MHz, DMSO-d₆) δ: ppm 11.06 (s, 1H), 8.18 (d, J=8.8 Hz, 1H), 7.56 (s, 1H), 7.03 (d, J=50.4 Hz, 1H), 4.50 (d, J=8.8 Hz, 1H), 4.02-3.89 (m, 2H), 3.86-3.78 (m, 1H), 3.75-3.67 (m, 1H), 3.46-3.39 (m, 2H), 3.15-3.06 (m, 2H), 2.76-2.65 (m, 1H), 2.60-2.53 (m, 1H), 2.45-2.35 (m, 1H), 1.94-1.70 (m, 6H), 1.67-1.55 (m, 2H), 1.49-1.41 (m, 1H), 1.39-1.20 (m, 3H), 0.95-0.85 (m, 6H), 0.75 (t, J=7.6 Hz, 3H), 0.67-0.57 (m, 4H).

MS obsd. (ESI⁺) [(M+H)⁺]: 570.4.

### Example 167b

¹H NMR (400 MHz, DMSO-d₆) δ: ppm 10.98 (s, 1H), 8.18 (d, J=8.8 Hz, 1H), 7.50 (s, 1H), 7.00 (d, J=50.4 Hz, 1H), 4.49 (d, J=8.8 Hz, 1H), 3.96-3.86 (m, 2H), 3.85-3.79 (m, 1H), 3.76-3.72 (m, 1H), 3.59-3.52 (m, 1H), 3.42-3.37 (m, 1H), 3.18-3.07 (m, 2H), 2.72-2.66 (m, 1H), 2.60-2.54 (m, 1H), 2.30-2.24 (m, 1H), 1.94-1.72 (m, 6H), 1.67-1.52 (m, 3H), 1.40-1.29 (m, 2H), 1.26-1.21 (m, 1H), 0.92-0.90 (m, 6H), 0.76 (t, J=7.6 Hz, 3H), 0.68-0.61 (m, 4H).

MS obsd. (ESI⁺) [(M+H)⁺]: 570.4.

### Example 168a and 168b

### N-[(1S)-1-[(2S)-2-[[[(2R)-2-chloro-2-fluoro-acetyl]-[[(3S)-2-oxo-3-piperidyl]methyl]amino]carbamoyl]-4,4-dimethyl-pyrrolidine-1-carbonyl]-2,2-dimethyl-butyl]cyclopropanecarboxamide and N-[(1S)-1-[(2S)-2-[[[(2R)-2-chloro-2-fluoro-acetyl]-[[(3R)-2-oxo-3-piperidyl]methyl]amino]carbamoyl]-4,4-dimethyl-pyrrolidine-1-carbonyl]-2,2-dimethyl-butyl]cyclopropanecarboxamide

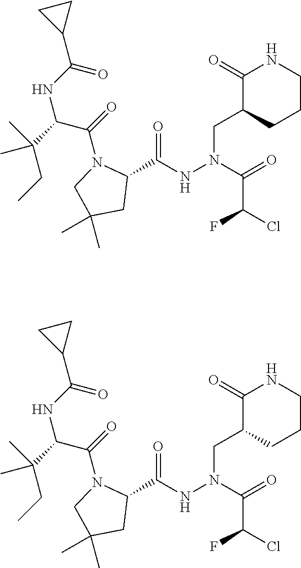 [SMILES: CCC(C)(C)[C@H](NC(=O)C1CC1)C(=O)N1CC(C)(C)C[C@H]1C(=O)NN(C[C@@H]1CCCNC1=O)C(=O)[C@H](F)Cl ; CCC(C)(C)[C@H](NC(=O)C1CC1)C(=O)N1CC(C)(C)C[C@H]1C(=O)NN(C[C@H]1CCCNC1=O)C(=O)[C@H](F)Cl]

The title compound was prepared in analogy to the procedure described for the preparation of Example 1, by using benzyl (2S)-1-[(2S)-2-amino-3,3-dimethyl-pentanoyl]-4,4-dimethyl-pyrrolidine-2-carboxylate; hydrochloride (Intermediate 35) instead of benzyl (JR, 2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[(2-oxo-3-piperidyl)methyl]carbamate (racemic, from Intermediate 39) instead of tert-butyl N-amino-N-[[(3S)-2-oxopyrrolidin-3-yl]methyl]carbamate (Intermediate 9), and cyclopropanecarbonyl chloride instead of TFAA to afford a mixture of N-[(1S)-1-[(2S)-2-[[[(2R)-2-chloro-2-fluoro-acetyl]-[[(3S)-2-oxo-3-piperidyl]methyl]amino]carbamoyl]-4,4-dimethyl-pyrrolidine-1-carbonyl]-2,2-dimethyl-butyl]cyclopropanecarboxamide and N-[(1S)-1-[(2S)-2-[[[(2R)-2-chloro-2-fluoro-acetyl]-[[(3R)-2-oxo-3-piperidyl]methyl]amino]carbamoyl]-4,4-dimethyl-pyrrolidine-1-carbonyl]-2,2-dimethyl-butyl]cyclopropanecarboxamide. The mixture was split by prep-SFC (DAICEL CHIRALPAK IC (250 mm*50 mm, 10 um); Condition Neu-ETOH Begin B 25, End B 25 Gradient Time (min) 3.7; 100% B Hold Time (min) FlowRate (mL/min) 60; Injections 35) to afford Example 168a and Example 168b as white solids.

### Example 168a

¹H NMR (400 MHz, DMSO-d₆) δ: ppm 10.97 (s, 1H), 8.18 (d, J=8.8 Hz, 1H), 7.56 (s, 1H), 7.08 (d, J=50.4 Hz, 1H), 4.47 (d, J=8.8 Hz, 1H), 4.27 (dd, J=10.0 Hz, 7.6 Hz, 1H), 3.95 (dd, J=14.0 Hz, 10.4 Hz, 1H), 3.71 (d, J=9.6 Hz, 1H), 3.37-3.35 (m, 1H), 3.31-3.26 (m, 1H), 3.15-3.05 (m, 2H), 2.44-2.34 (m, 1H), 1.95-1.84 (m, 2H), 1.83-1.71 (m, 2H), 1.65-1.52 (m, 2H), 1.50-1.32 (m, 2H), 1.27-1.17 (m, 1H), 1.13 (s, 3H), 0.97 (s, 3H), 0.94-0.86 (m, 6H), 0.75 (t, J=7.2 Hz, 3H), 0.67-0.55 (m, 4H).

MS obsd. (ESI⁺) [(M+H)⁺]: 558.4.

### Example 168b

¹H NMR (400 MHz, DMSO-d₆) δ: ppm 10.90 (s, 1H), 8.17 (d, J=8.8 Hz, 1H), 7.51 (s, 1H), 7.08 (d, J=50.0 Hz, 1H), 4.44 (d, J=8.4 Hz, 1H), 4.30 (dd, J=10.0 Hz, 7.6 Hz, 1H), 3.85 (dd, J=14.0 Hz, 10.4 Hz, 1H), 3.71 (d, J=9.6 Hz, 1H), 3.62-3.54 (m, 1H), 3.30-3.26 (m, 1H), 3.18-3.04 (m, 2H), 2.49-2.18 (m, 1H), 1.95-1.84 (m, 3H), 1.80-1.75 (m, 1H), 1.67-1.45 (m, 3H), 1.41-1.32 (m, 1H), 1.25-1.19 (m, 1H), 1.12 (s, 3H), 0.97 (s, 3H), 0.93-0.87 (m, 6H), 0.79-0.73 (m, 3H), 0.68-0.56 (m, 4H).

MS obsd. (ESI⁺) [(M+H)⁺]: 558.4.

### Example 169a and 169b

### N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide and N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide

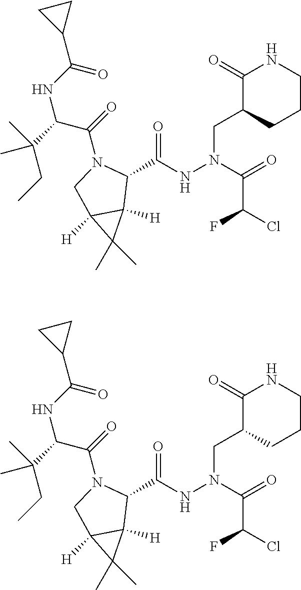 [SMILES: CCC(C)(C)[C@H](NC(=O)C1CC1)C(=O)N1C[C@H]2[C@@H]([C@H]1C(=O)NN(C[C@@H]1CCCNC1=O)C(=O)[C@H](F)Cl)C2(C)C ; CCC(C)(C)[C@H](NC(=O)C1CC1)C(=O)N1C[C@H]2[C@@H]([C@H]1C(=O)NN(C[C@H]1CCCNC1=O)C(=O)[C@H](F)Cl)C2(C)C]

The title compound was prepared in analogy to the procedure described for the preparation of Example 1, by using benzyl (1R,2S,5S)-3-[(2S)-2-amino-3,3-dimethyl-pentanoyl]-6,6-dimethyl-3-azabicyclo[3.1.0]hexane-2-carboxylate; 2,2,2-trifluoroacetic acid (Intermediate 40) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[(2-oxo-3-piperidyl)methyl]carbamate (racemic, from Intermediate 39) instead of tert-butyl N-amino-N-[[(3S)-2-oxopyrrolidin-3-yl]methyl]carbamate (Intermediate 9), and cyclopropanecarbonyl chloride instead of TFAA to afford a mixture of N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide and N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide. The mixture was split by prep-SFC (Instrument:Waters 80Q; Mobile Phase: 35% EtOH (Neu) in Supercritical CO₂; Flow Rate: 60 g/min; Cycle Time: 3.6 min, total time: 25 min; Single injection volume: 3.5 ml; Back Pressure: 100 bar to keep the CO₂ in Supercritical flow) to afford Example 169a (retention time=1.84 min) and Example 169b (retention time=3.69 min) as white solids.

### Example 169a

¹H NMR (400 MHz, DMSO-d₆) δ: ppm 11.13 (s, 1H), 8.17 (d, J=7.2 Hz, 1H), 7.56 (s, 1H), 6.91 (d, J=50.8 Hz, 1H), 4.44 (d, J=8.8 Hz, 1H), 4.12 (s, 1H), 3.99 (dd, J=13.6 Hz, 10.0 Hz, 1H), 3.92-3.80 (m, 2H), 3.46-3.41 (m, 1H), 3.16-3.06 (m, 2H), 2.48-2.42 (m, 1H), 1.90-1.75 (m, 3H), 1.67-1.54 (m, 2H), 1.53-1.39 (m, 2H), 1.35-1.20 (m, 2H), 1.04 (s, 3H), 0.91 (s, 6H), 0.87 (s, 3H), 0.79-0.73 (m, 3H), 0.70-0.60 (m, 3H), 0.59-0.50 (m, 1H).

MS obsd. (ESI⁺) [(M+H)⁺]: 570.3.

### Example 169b

¹H NMR (400 MHz, DMSO-d₆) δ: ppm 11.08 (s, 1H), 8.17-8.10 (m, 1H), 7.52 (s, 1H), 6.90 (d, J=50.4 Hz, 1H), 4.42 (d, J=8.8 Hz, 1H), 4.14 (s, 1H), 3.95-3.80 (m, 3H), 3.63 (dd, J=13.6 Hz, 8.8 Hz, 1H), 3.47-3.39 (m, 1H), 3.17-3.10 (m, 2H), 2.40-2.30 (m, 1H), 1.95-1.75 (m, 3H), 1.65-1.52 (m, 3H), 1.48 (d, J=7.6 Hz, 1H), 1.37-1.20 (m, 2H), 1.03 (s, 3H), 0.91 (d, J=2.0 Hz, 6H), 0.87 (s, 2H), 0.76 (t, J=7.2 Hz, 3H), 0.71-0.60 (m, 3H), 0.59-0.48 (m, 1H).

MS obsd. (ESI⁺) [(M+H)⁺]: 570.3.

### Example 170a and 170b

### N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide and N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide

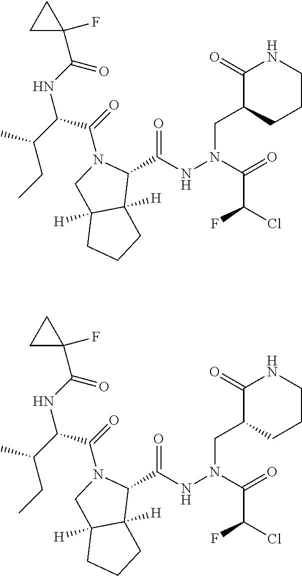 [SMILES: CC[C@H](C)[C@H](NC(=O)C1(F)CC1)C(=O)N1C[C@@H]2CCC[C@@H]2[C@H]1C(=O)NN(C[C@@H]1CCCNC1=O)C(=O)[C@H](F)Cl ; CC[C@H](C)[C@H](NC(=O)C1(F)CC1)C(=O)N1C[C@@H]2CCC[C@@H]2[C@H]1C(=O)NN(C[C@H]1CCCNC1=O)C(=O)[C@H](F)Cl]

The title compound was prepared in analogy to the procedure described for the preparation of Example 1, by using benzyl (3S,3aS,6aR)-2-[(2S,3S)-2-amino-3-methyl-pentanoyl]-3,3a,4,5,6,6a-hexahydro-1H-cyclopenta[c]pyrrole-3-carboxylate; hydrochloride (Intermediate 41) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[(2-oxo-3-piperidyl)methyl]carbamate (racemic, from Intermediate 39) instead of tert-butyl N-amino-N-[[(3S)-2-oxopyrrolidin-3-yl]methyl]carbamate (Intermediate 9), and 1-fluorocyclopropane-1-carboxylic acid and T₃P instead of TFAA to afford a mixture of N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide and N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide. The mixture was split by prep-SFC (Column: Chiralpak IG-3 50×4.6 mm I.D., 3 um Mobile phase: Phase A for C₀₂, and Phase B for EtOH (0.05% DEA); Gradient elution: EtOH (0.05% DEA) in CO₂ from 5% to 20% Flow rate: 3 mL/min; Detector: PDA Column Temp: 35C; Back Pressure: 100 Bar; YMC-Actus Triart C18 150*30 mm*7 um, water (ammonia hydroxide v/v)-ACN, Gradient Time: 4.9 min; FlowRate: 70 mL/min) to afford Example 170a (retention time=3.59 min) and Example 170b (retention time=4.58 min) as a white solid.

### Example 170a

¹H NMR (400 MHz, DMSO-d₆) δ: ppm 10.96 (s, 1H), 8.44-8.29 (m, 1H), 7.49 (s, 1H), 6.92 (d, J=50.4 Hz, 1H), 4.30 (t, J=8.4 Hz, 1H), 3.99 (d, J=6.0 Hz, 1H), 3.95-3.87 (m, 1H), 3.87-3.77 (m, 2H), 3.58 (dd, J=14.0 Hz, 8.8 Hz, 1H), 3.17-3.08 (m, 2H), 2.77-2.68 (m, 1H), 2.61-2.55 (m, 1H), 2.34-2.25 (m, 1H), 1.98-1.87 (m, 2H), 1.86-1.67 (m, 5H), 1.64-1.46 (m, 4H), 1.40-1.30 (m, 2H), 1.29-1.26 (m, 1H), 1.25-1.17 (m, 1H), 1.16-1.04 (m, 2H), 0.88-0.78 (m, 6H).

MS obsd. (ESI⁺) [(M+H)⁺]: 574.2.

### Example 170b

¹H NMR (400 MHz, DMSO-d₆) δ: ppm 11.03 (br. s, 1H), 8.42-8.25 (m, 1H), 7.56-7.45 (m, 1H), 6.92 (d, J=50.4 Hz, 1H), 4.30 (t, J=8.4 Hz, 1H), 4.02-3.91 (m, 2H), 3.87-3.77 (m, 2H), 3.15-3.08 (m, 2H), 2.78-2.69 (m, 1H), 2.44-2.37 (m, 1H), 1.97-1.89 (m, 1H), 1.87-1.67 (m, 6H), 1.65-1.55 (m, 2H), 1.53-1.44 (m, 2H), 1.42-1.27 (m, 3H), 1.27-0.99 (m, 5H), 0.89-0.77 (m, 6H).

MS obsd. (ESI⁺) [(M+H)⁺]: 574.2.

### Example 171a and 171b

### N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide and N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide

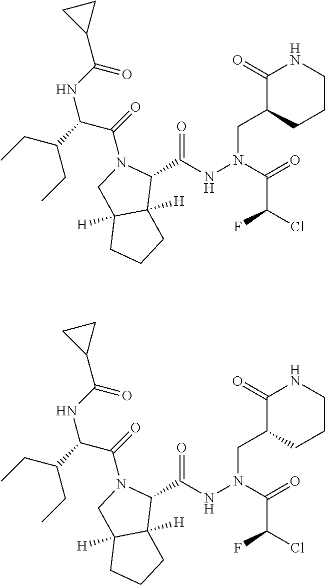 [SMILES: CCC(CC)[C@H](NC(=O)C1CC1)C(=O)N1C[C@@H]2CCC[C@@H]2[C@H]1C(=O)NN(C[C@@H]1CCCNC1=O)C(=O)[C@H](F)Cl ; CCC(CC)[C@H](NC(=O)C1CC1)C(=O)N1C[C@@H]2CCC[C@@H]2[C@H]1C(=O)NN(C[C@H]1CCCNC1=O)C(=O)[C@H](F)Cl]

The title compound was prepared in analogy to the procedure described for the preparation of Example 1, by using benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[(2-oxo-3-piperidyl)methyl]carbamate (racemic, from Intermediate 39) instead of tert-butyl N-amino-N-[[(3S)-2-oxopyrrolidin-3-yl]methyl]carbamate (Intermediate 9), and cyclopropanecarbonyl chloride instead of TFAA to afford a mixture of N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide and N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide. The mixture was split by prep-SFC (Instrument:Waters 80Q; Mobile Phase: 40% EtOH (Neu) in Supercritical CO₂; Flow Rate: 70 g/min; Cycle Time: 3.2 min, total time: 25 min; Single injection volume: 3.5 mL; Back Pressure: 100 bar to keep the CO₂ in Supercritical flow.) to afford Example 171a (retention time=1.64 min) and Example 171b (retention time=3.18 min) as white solids.

### Example 171a

¹H NMR (400 MHz, DMSO-d₆) δ: ppm 11.06 (s, 1H), 8.36-8.29 (m, 1H), 7.58-7.50 (m, 1H), 6.95 (d, J=50.8 Hz, 1H), 4.52 (t, J=8.4 Hz, 1H), 4.02-3.91 (m, 2H), 3.84-3.69 (m, 2H), 3.37-3.34 (m, 1H), 3.15-3.09 (m, 2H), 2.80-2.71 (m, 1H), 2.45-2.36 (m, 1H), 1.91-1.76 (m, 4H), 1.75-1.69 (m, 3H), 1.68-1.49 (m, 4H), 1.48-1.40 (m, 2H), 1.37-1.30 (m, 1H), 1.29-1.20 (m, 3H), 0.83-0.79 (m, 3H), 0.76 (t, J=7.6 Hz, 3H), 0.69-0.59 (m, 4H).

MS obsd. (ESI⁺) [(M+H)⁺]: 570.4.

### Example 171b

¹H NMR (400 MHz, DMSO-d₆) δ: ppm 10.97 (s, 1H), 8.36-8.27 (d, J=8.8 Hz, 1H), 7.50 (s, 1H), 6.93 (d, J=50.4 Hz, 1H), 4.50 (t, J=8.4 Hz, 1H), 3.99 (d, J=6.0 Hz, 1H), 3.91 (dd, J=14.0 Hz, 4.4 Hz, 1H), 3.82-3.72 (m, 2H), 3.57 (dd, J=14.0 Hz, 8.4 Hz, 1H), 3.19-3.07 (m, 2H), 2.78-2.69 (m, 1H), 2.61-2.55 (m, 1H), 2.33-2.23 (m, 1H), 1.95-1.70 (m, 7H), 1.66-1.58 (m, 2H), 1.57-1.47 (m, 2H), 1.45-1.31 (m, 2H), 1.30-1.22 (m, 3H), 0.85-0.79 (m, 3H), 0.78-0.72 (m, 3H), 0.68-0.58 (m, 4H).

MS obsd. (ESI⁺) [(M+H)⁺]: 570.4.

### Example 172

### N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]cyclopropanecarboxamide

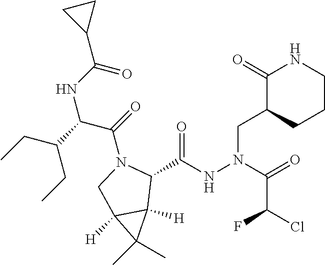 [SMILES: CCC(CC)[C@H](NC(=O)C1CC1)C(=O)N1C[C@H]2[C@@H]([C@H]1C(=O)NN(C[C@@H]1CCCNC1=O)C(=O)[C@H](F)Cl)C2(C)C]

The title compound was prepared in analogy to the procedure described for the preparation of Example 1, by using benzyl (1R,2S,5S)-3-[(2S)-2-amino-3-ethyl-pentanoyl]-6,6-dimethyl-3-azabicyclo[3.1.0]hexane-2-carboxylate; hydrochloride (Intermediate 31) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9), and cyclopropanecarbonyl chloride instead of TFAA to afford N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]cyclopropanecarboxamide (Example 172) as a white solid.

¹H NMR (400 MHz, DMSO-d₆) δ: ppm 11.12 (s, 1H), 8.35 (d, J=8.8 Hz, 1H), 7.55 (s, 1H), 6.84 (d, J=50.8 Hz, 1H), 4.40 (t, J=8.8 Hz, 1H), 4.13 (s, 1H), 3.99 (dd, J=10.0, 14.0 Hz, 1H), 3.91-3.85 (m, 1H), 3.79 (dd, J=10.4 Hz, 5.6 Hz, 1H), 3.40 (dd, J=13.6 Hz, 4.0 Hz, 1H), 3.14-3.07 (m, 2H), 1.86-1.74 (m, 2H), 1.70-1.60 (m, 3H), 1.51-1.38 (m, 3H), 1.30-1.19 (m, 5H), 1.03 (s, 3H), 0.87 (s, 3H), 0.81-0.71 (m, 6H), 0.69-0.60 (m, 3H), 0.59-0.52 (m, 1H).

MS obsd. (ESI⁺) [(M+H)⁺]: 570.3.

### Example 173

### N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]-2,2-difluoro-acetamide

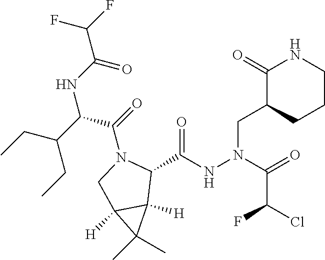 [SMILES: CCC(CC)[C@H](NC(=O)C(F)F)C(=O)N1C[C@H]2[C@@H]([C@H]1C(=O)NN(C[C@@H]1CCCNC1=O)C(=O)[C@H](F)Cl)C2(C)C]

The title compound was prepared in analogy to the procedure described for the preparation of Example 1, by using benzyl (1R,2S,5S)-3-[(2S)-2-amino-3-ethyl-pentanoyl]-6,6-dimethyl-3-azabicyclo[3.1.0]hexane-2-carboxylate; hydrochloride (Intermediate 31) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9), and (2,2-difluoroacetyl) 2,2-difluoroacetate instead of TFAA to afford N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]-2,2-difluoro-acetamide (Example 173) as a white solid.

¹H NMR (400 MHz, DMSO-d₆) δ: ppm 11.14 (s, 1H), 9.17 (d, J=8.4 Hz, 1H), 7.55 (s, 1H), 6.80 (d, J=50.8 Hz, 1H), 6.22 (t, J=53.6 Hz, 1H), 4.43 (t, J=8.4 Hz, 1H), 4.16 (s, 1H), 4.00 (dd, J=13.6 Hz, 10.0 Hz, 1H), 3.90-3.80 (m, 2H), 3.42 (dd, J=14.0, 4.0 Hz, 1H), 3.14-3.07 (m, 2H), 2.48-2.43 (m, 1H), 1.86-1.71 (m, 3H), 1.70-1.63 (m, 1H), 1.63-1.55 (m, 1H), 1.53-1.38 (m, 3H), 1.31-1.21 (m, 3H), 1.05 (s, 3H), 0.90 (s, 3H), 0.81 (t, J=7.2 Hz, 3H), 0.75 (t, J=7.2 Hz, 3H).

MS obsd. (ESI⁺) [(M+H)⁺]: 580.2.

### Example 174

### N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]-2,2-difluoro-acetamide

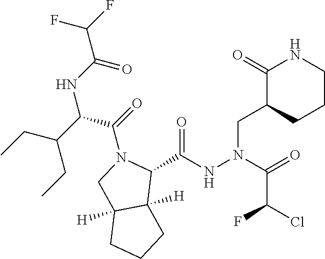 [SMILES: CCC(CC)[C@H](NC(=O)C(F)F)C(=O)N1C[C@@H]2CCC[C@@H]2[C@H]1C(=O)NN(C[C@@H]1CCCNC1=O)C(=O)[C@H](F)Cl]

The title compound was prepared in analogy to the procedure described for the preparation of Example 1, by using benzyl (3S,3aS,6aR)-2-[(2S)-2-amino-3-ethyl-pentanoyl]-3,3a,4,5,6,6a-hexahydro-1H-cyclopenta[c]pyrrole-3-carboxylate; 2,2,2-trifluoroacetic acid (Intermediate 24) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9), and (2,2-difluoroacetyl) 2,2-difluoroacetate instead of TFAA to afford N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]-2,2-difluoro-acetamide (Example 174) as a white solid.

¹H NMR (400 MHz, DMSO-d₆) δ: ppm 11.08 (s, 1H), 9.13-9.02 (m, 1H), 7.57-7.48 (m, 1H), 6.88 (d, J=50.8 Hz, 1H), 6.26 (t, J=53.6 Hz, 1H), 4.56 (t, J=8.0 Hz, 1H), 3.99 (d, J=5.2 Hz, 1H), 3.98-3.93 (m, 1H), 3.87-3.80 (m, 1H), 3.73 (dd, J=7.6 Hz, 2.8 Hz, 1H), 3.12-3.09 (m, 2H), 2.85-2.70 (m, 1H), 2.45-2.36 (m, 1H), 1.90-1.78 (m, 3H), 1.78-1.67 (m, 4H), 1.67-1.48 (m, 3H), 1.48-1.36 (m, 3H), 1.31-1.15 (m, 4H), 0.86-0.80 (t, J=7.2 Hz, 3H), 0.76 (t, J=7.6 Hz, 3H).

MS obsd. (ESI⁺) [(M+H)⁺]: 580.3.

### Example 175

### N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide

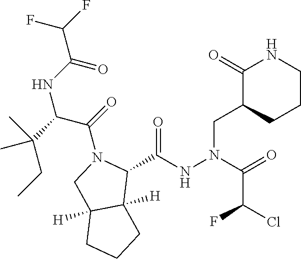 [SMILES: CCC(C)(C)[C@H](NC(=O)C(F)F)C(=O)N1C[C@@H]2CCC[C@@H]2[C@H]1C(=O)NN(C[C@@H]1CCCNC1=O)C(=O)[C@H](F)Cl]

The title compound was prepared in analogy to the procedure described for the preparation of Example 1, by using benzyl (3S,3aS,6aR)-2-[(2S)-2-amino-3,3-dimethyl-pentanoyl]-3,3a,4,5,6,6a-hexahydro-1H-cyclopenta[c]pyrrole-3-carboxylate (Intermediate 34) instead of benzyl (1R,2S,5S)-3-[(2S)-2-amino-3,3-dimethyl-butanoyl]-6,6-dimethyl-3-azabicyclo[3.1.0]hexane-2-carboxylate (Intermediate 1), tert-butyl N-amino-N-[[(3S)-2-oxo-3-piperidyl]methyl]carbamate (Intermediate 39) instead of tert-butyl N-amino-N-[[(3S)-2-oxopyrrolidin-3-yl]methyl]carbamate (Intermediate 9), and (2,2-difluoroacetyl) 2,2-difluoroacetate instead of TFAA to afford N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide (Example 175) as a white solid.

¹H NMR (400 MHz, DMSO-d₆) δ: ppm 11.08 (s, 1H), 9.13-9.02 (m, 1H), 7.57-7.48 (m, 1H), 6.88 (d, J=50.8 Hz, 1H), 6.26 (t, J=53.6 Hz, 1H), 4.56 (t, J=8.0 Hz, 1H), 3.99 (d, J=5.2 Hz, 1H), 3.98-3.93 (m, 1H), 3.87-3.80 (m, 1H), 3.73 (dd, J=7.6 Hz, 2.8 Hz, 1H), 3.12-3.09 (m, 2H), 2.85-2.70 (m, 1H), 2.45-2.36 (m, 1H), 1.90-1.78 (m, 3H), 1.78-1.67 (m, 4H), 1.67-1.48 (m, 3H), 1.48-1.36 (m, 3H), 1.31-1.15 (m, 4H), 0.86-0.80 (t, J=7.2 Hz, 3H), 0.76 (t, J=7.6 Hz, 3H).

MS obsd. (ESI⁺) [(M+H)⁺]: 580.3.

### Example 176

### N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2-difluoro-acetamide

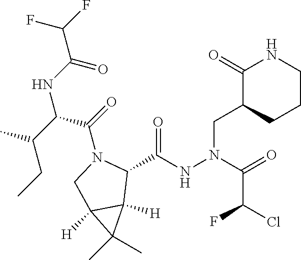

The title compound was prepared in analogy to the procedure described for the preparation of Example 105, by using tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9), and (2,2-difluoroacetyl) 2,2-difluoroacetate without coupling reagent instead of 1-fluorocyclobutanecarboxylic acid to afford N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2-difluoro-acetamide (Example 176) as a white solid.

¹H NMR (400 MHz, DMSO-d₆) δ: ppm 11.13 (s, 1H), 9.25 (d, J=8.0 Hz, 1H), 7.55 (s, 1H), 6.83 (d, J=50.8 Hz, 1H), 6.20 (t, J=54.0 Hz, 1H), 4.21 (t, J=9.6 Hz, 1H), 4.13 (s, 1H), 4.01-3.96 (m, 1H), 3.91-3.83 (m, 2H), 3.44 (dd, J=14.0 Hz, 4.4 Hz, 1H), 3.13-3.10 (m, 2H), 2.47-2.42 (m, 1H), 1.87-1.74 (m, 3H), 1.68-1.63 (m, 1H), 1.62-1.55 (m, 1H), 1.54-1.42 (m, 3H), 1.14-1.03 (m, 4H), 0.94-0.78 (m, 9H).

MS obsd. (ESI⁺) [(M+H)⁺]: 566.3.

### Example 177

### (1R,2S,5S)-3-[3,5-bis(trifluoromethyl)benzoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide

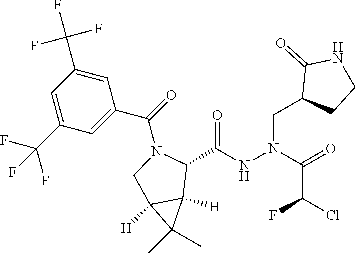 [SMILES: CC1(C)[C@@H]2[C@@H](C(=O)NN(C[C@@H]3CCNC3=O)C(=O)[C@H](F)Cl)N(C(=O)c3cc(C(F)(F)F)cc(C(F)(F)F)c3)C[C@@H]21]

### Step 1: Preparation of tert-butyl N-[[(1R,2S,5S)-3-[3,5-bis(trifluoromethyl)benzoyl]-6,6-dimethyl-3-azabicyclo[3.1.0]hexane-2-carbonyl]amino]-N-[[(3S)-2-oxopyrrolidin-3-yl]methyl]carbamate

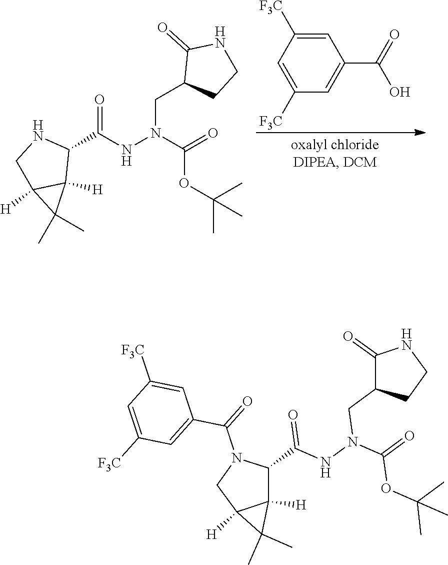

To a solution of 3,5-bis(trifluoromethyl)benzoic acid (620 mg, 2.4 mmol) in DCM (5 mL) was added DMF (7.98 mg, 0.11 mmol) and oxalyl chloride (416 mg, 3.27 mmol). The mixture was stirred at 25° C. for 1 h and concentrated in vacuum. The residue was dissolved with DCM (4 mL) and added into the solution of tert-butyl N-[[(1R,2S,5S)-6,6-dimethyl-3-azabicyclo[3.1.0]hexane-2-carbonyl]amino]-N-[[(3S)-2-oxopyrrolidin-3-yl]methyl]carbamate (400 mg, 1.09 mmol, Intermediate 14) and DIPEA (705.34 mg, 5.46 mmol) in DCM (5 mL) at 0° C. The reaction mixture was stirred at 25° C. for 2 h. The resulting mixture was poured into water (20 mL) and extracted with DCM (50 mL×2). The combined organic phase was washed with 10% aqueous solution of NaHCO₃ (10 mL), brine (10 mL), dried over Na₂SO₄ and concentrated in vacuum. The residue was purified by reverse flash chromatography (condition: 120 g Flash Column Welch Ultimate XB_C18 20-40 μm; 120 A, water (0.1% FA)-ACN, 0˜60%, 70 mL/min) to afford tert-butyl N-[[(1R,2S,5S)-3-[3,5-bis(trifluoromethyl)benzoyl]-6,6-dimethyl-3-azabicyclo[3.1.0]hexane-2-carbonyl]amino]-N-[[(3S)-2-oxopyrrolidin-3-yl]methyl]carbamate (500 mg) as a white solid.

MS obsd. (ESI⁺) [(M+H)⁺]: 607.3.

### Step 2: Preparation of (1R,2S,5S)-3-[3,5-bis(trifluoromethyl)benzoyl]-6,6-dimethyl-N′-[[(3S)-2-oxopyrrolidin-3-yl]methyl]-3-azabicyclo[3.1.0]hexane-2-carbohydrazide

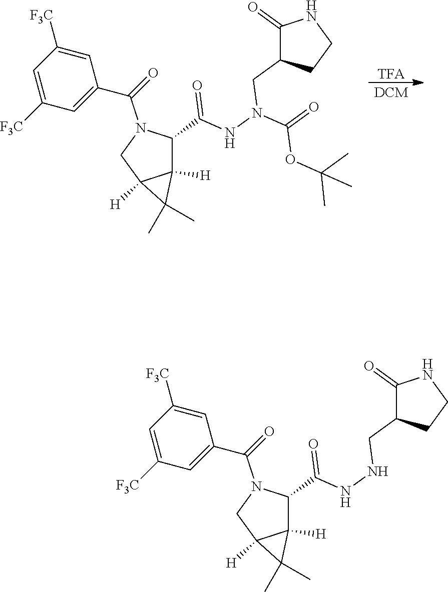

To a solution of tert-butyl N-[[(1R,2S,5S)-3-[3,5-bis(trifluoromethyl)benzoyl]-6,6-dimethyl-3-azabicyclo[3.1.0]hexane-2-carbonyl]amino]-N-[[(3S)-2-oxopyrrolidin-3-yl]methyl]carbamate (500 mg, 0.82 mmol) in DCM (4 mL) was added TFA (2.0 mL). The mixture was stirred at 25° C. for 1 h and concentrated in vacuum. The residue was purified by reverse flash chromatography (condition: 120 g Flash Column Welch Ultimate XB_C18 20-40 μm; 120 A, water (0.1% HCl)-ACN, 0˜60%, 70 mL/min) to afford (1R,2S,5S)-3-[3,5-bis(trifluoromethyl)benzoyl]-6,6-dimethyl-N′—[[(3S)-2-oxopyrrolidin-3-yl]methyl]-3-azabicyclo[3.1.0]hexane-2-carbohydrazide; hydrochloride (350 mg) as a white solid MS obsd. (ESI⁺) [(M+H)⁺]: 507.1.

### Step 3: Preparation of (1R,2S,5S)-3-[3,5-bis(trifluoromethyl)benzoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide

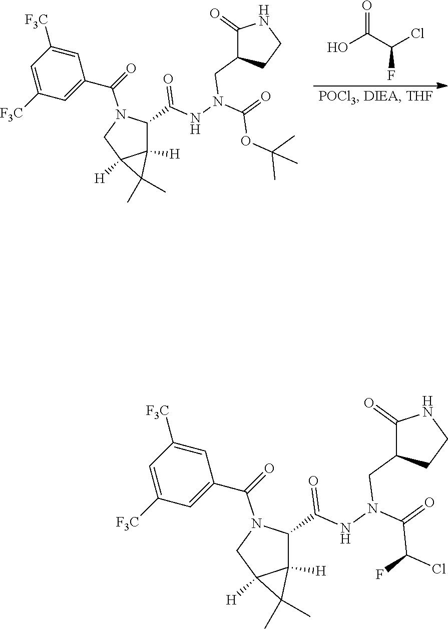

To a solution of (2R)-2-chloro-2-fluoro-acetic acid (222 mg, 1.38 mmol, 70% purity, Intermediate 10) in DCM (5 mL) was added POCl₃ (190 mg, 1.2 mmol). The mixture was stirred for 90 min at 25° C. The mixture was added to the solution of (1R,2S,5S)-3-[3,5-bis(trifluoromethyl)benzoyl]-6,6-dimethyl-N′—[[(3S)-2-oxopyrrolidin-3-yl]methyl]-3-azabicyclo[3.1.0]hexane-2-carbohydrazide; hydrochloride (350 mg, 0.69 mmol) and DIPEA (536 mg, 4.15 mmol) in DCM (5 mL) at −5° C. After addition, the mixture was stirred at −5° C. for 1 h. The reaction mixture was quenched with MeOH (5 mL) and acidified by 4 N HCl/dioxane to pH=5 at −10° C. Then the mixture was concentrated in vacuum at 30° C. The residue was purified by reversed flash chromatography (condition: 120 g Flash Column Welch Ultimate XB_C18 20-40 μm; 120 A, water (0.1% FA)-ACN, 0˜60%, 70 mL/min) to afford (1R,2S,5S)-3-[3,5-bis(trifluoromethyl)benzoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide (Example 177) (285 mg) as a white solid.

¹H NMR (400 MHz, DMSO-d₆) δ: ppm 11.16-10.63 (m, 1H), 8.29 (s, 1H), 8.23-8.11 (m, 2H), 7.76 (s, 1H), 6.83 (d, J=50.0 Hz, 1H), 4.48-4.38 (m, 1H), 4.03-3.88 (m, 1H), 3.84 (dd, J=14.0 Hz, 7.2 Hz, 1H), 3.50 (d, J=14.0 Hz, 4.4 Hz, 1H), 3.29-3.27 (m, 1H), 3.19-3.13 (m, 2H), 2.63-2.57 (m, 1H), 2.21-2.13 (m, 1H), 1.87-1.74 (m, 1H), 1.69-1.57 (m, 2H), 1.09-0.92 (m, 6H).

MS obsd. (ESI⁺) [(M+H)⁺]: 601.3.

### Example 178

### (1R,2S,5S)-3-[2-[3,5-bis(trifluoromethyl)phenyl]acetyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide

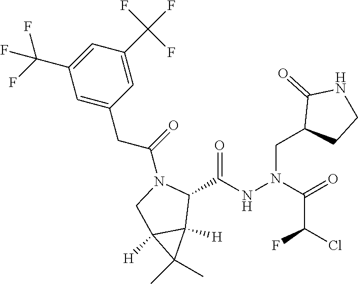 [SMILES: CC1(C)[C@@H]2[C@@H](C(=O)NN(C[C@@H]3CCNC3=O)C(=O)[C@H](F)Cl)N(C(=O)Cc3cc(C(F)(F)F)cc(C(F)(F)F)c3)C[C@@H]21]

The title compound was prepared in analogy to the procedure described for the preparation of Example 177, by using 3,5-bis(trifluoromethyl)phenylacetic acid instead of 3,5-bis(trifluoromethyl)benzoic acid to afford (1R,2S,5S)-3-[2-[3,5-bis(trifluoromethyl)phenyl]acetyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide (Example 178) as a white solid.

¹H NMR (400 MHz, DMSO-d₆) δ: ppm 11.03 (s, 1H), 7.97 (s, 1H), 7.95-7.89 (m, 2H), 7.80-7.67 (m, 1H), 6.75 (d, J=50.0 Hz, 1H), 4.14 (s, 1H), 4.03-3.92 (m, 2H), 3.91-3.74 (m, 2H), 3.62 (d, J=10.4 Hz, 1H), 3.37 (dd, J=14.0 Hz, 4.4 Hz, 1H), 3.20-3.02 (m, 2H), 2.62-2.54 (m, 1H), 2.22-2.06 (m, 1H), 1.81-1.70 (m, 1H), 1.68-1.60 (m, 1H), 1.59-1.49 (m, 1H), 1.05 (s, 3H), 0.97-0.85 (m, 3H).

MS obsd. (ESI⁺) [(M+H)⁺]: 615.2.

### Example 179

### (1R,2S,5S)—N′-[(2R)-2-chloro-2-fluoro-acetyl]-3-[3-fluoro-5-(trifluoromethyl)benzoyl]-6,6-dimethyl-N′—[[(3S)-2-oxopyrrolidin-3-yl]methyl]-3-azabicyclo[3.1.0]hexane-2-carbohydrazide

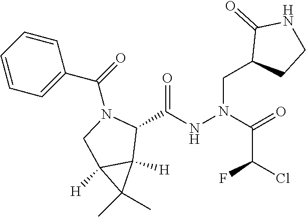 [SMILES: CC1(C)[C@@H]2[C@@H](C(=O)NN(C[C@@H]3CCNC3=O)C(=O)[C@H](F)Cl)N(C(=O)c3ccccc3)C[C@@H]21]

The title compound was prepared in analogy to the procedure described for the preparation of Example 177, by using 3-fluoro-5-(trifluoromethyl)benzoyl chloride instead of 3,5-bis(trifluoromethyl) benzoyl chloride to afford (1R,2S,5S)—N′-[(2R)-2-chloro-2-fluoro-acetyl]-3-[3-fluoro-5-(trifluoromethyl)benzoyl]-6,6-dimethyl-N-[[(3S)-2-oxopyrrolidin-3-yl]methyl]-3-azabicyclo[3.1.0]hexane-2-carbohydrazide (Example 179) as a white solid.

¹H NMR (400 MHz, DMSO-d₆) δ: ppm 11.16 (br. s, 1H), 7.87 (d, J=19.2 Hz, 1H), 7.83-7.71 (m, 2H), 7.65-7.60 (m, 1H), 6.83 (d, J=50.4 Hz, 1H), 4.44-4.36 (m, 1H), 3.89-3.83 (m, 2H), 3.54-3.47 (m, 3H), 3.14-3.10 (m, 2H), 2.26-2.15 (m, 1H), 1.79-1.74 (m, 1H), 1.70-1.58 (m, 2H), 1.12-0.91 (m, 6H).

MS obsd. (ESI⁺) [(M+H)⁺]: 551.2.

### Example 180

### (1R,2S,5S)-3-[(E)-3-[3,5-bis(trifluoromethyl)phenyl]prop-2-enoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide

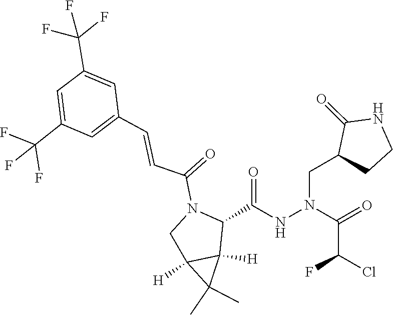 [SMILES: CC1(C)[C@@H]2[C@@H](C(=O)NN(C[C@@H]3CCNC3=O)C(=O)[C@H](F)Cl)N(C(=O)/C=C/c3cc(C(F)(F)F)cc(C(F)(F)F)c3)C[C@@H]21]

The title compound was prepared in analogy to the procedure described for the preparation of Example 177, by using (E)-3-[3,5-bis(trifluoromethyl)phenyl]prop-2-enoic acid instead of 3,5-bis(trifluoromethyl)benzoic acid to afford (1R,2S,5S)-3-[(E)-3-[3,5-bis(trifluoromethyl)phenyl]prop-2-enoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide (Example 180) as a white solid.

¹H NMR (400 MHz, DMSO-d₆) δ: ppm 11.20 (s, 1H), 8.53 (s, 2H), 8.08 (s, 1H), 7.76 (s, 1H), 7.68 (d, J=15.6 Hz, 1H), 7.34 (d, J=15.2 Hz, 1H), 6.91 (d, J=50.4 Hz, 1H), 4.25 (s, 1H), 3.99-3.92 (m, 2H), 3.85 (dd, J=14.0 Hz, 9.6 Hz, 1H), 3.42 (dd, J=14.0 Hz, 4.4 Hz, 1H), 3.19-3.15 (m, 2H), 2.65-2.56 (m, 1H), 2.21-2.13 (m, 1H), 1.83-1.75 (m, 1H), 1.71-1.62 (m, 1H), 1.58-1.55 (d, J=7.6 Hz, 1H), 1.08 (s, 3H), 0.99-0.93 (m, 3H).

MS obsd. (ESI⁺) [(M+H)⁺]: 627.2.

### Example 181

### (1R,2S,5S)—N′-[(2R)-2-chloro-2-fluoro-acetyl]-3-[(E)-3-(3,5-difluorophenyl)prop-2-enoyl]-6,6-dimethyl-N′—[[(3S)-2-oxopyrrolidin-3-yl]methyl]-3-azabicyclo[3.1.0]hexane-2-carbohydrazide

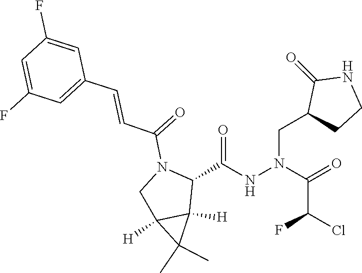 [SMILES: CC1(C)[C@@H]2[C@@H](C(=O)NN(C[C@@H]3CCNC3=O)C(=O)[C@H](F)Cl)N(C(=O)/C=C/c3cc(F)cc(F)c3)C[C@@H]21]

The title compound was prepared in analogy to the procedure described for the preparation of Example 177, by using (E)-3-(3,5-difluorophenyl)prop-2-enoic acid instead of 3,5-bis(trifluoromethyl)benzoic acid to afford (1R,2S,5S)—N-[(2R)-2-chloro-2-fluoro-acetyl]-3-[(E)-3-(3,5-difluorophenyl)prop-2-enoyl]-6,6-dimethyl-N-[[(3S)-2-oxopyrrolidin-3-yl]methyl]-3-azabicyclo[3.1.0]hexane-2-carbohydrazide (Example 181) as a white solid.

¹H NMR (400 MHz, DMSO-d₆) δ: ppm 11.11 (s, 1H), 7.75 (s, 1H), 7.58 (d, J=6.8 Hz, 2H), 7.46 (d, J=15.6 Hz, 1H), 7.29-7.23 (m, 1H), 7.10 (d, J=15.2 Hz, 1H), 6.91 (d, J=50.4 Hz, 1H), 4.23 (s, 1H), 3.96-3.92 (m, 1H), 3.86-3.81 (m, 2H), 3.44-3.39 (m, 1H), 3.18-3.12 (m, 2H), 2.62-2.56 (m, 1H), 2.19-2.11 (m, 1H), 1.82-1.74 (m, 1H), 1.68-1.63 (m, 1H), 1.55 (d, J=7.6 Hz, 1H), 1.07 (s, 3H), 0.96-0.91 (m, 3H).

MS obsd. (ESI⁺) [(M+H)⁺]: 527.2.

### Example 182

### (3S,3aS,6aR)—N′-[(2R)-2-chloro-2-fluoro-acetyl]-2-[(2S)-2-[(6-fluoropyrimidin-4-yl)amino]-3,3-dimethyl-butanoyl]-N-[[(3S)-2-oxopyrrolidin-3-yl]methyl]-3,3a,4,5,6,6a-hexahydro-1H-cyclopenta[c]pyrrole-3-carbohydrazide

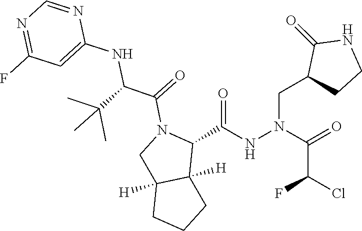 [SMILES: CC(C)(C)[C@H](Nc1cc(F)ncn1)C(=O)N1C[C@@H]2CCC[C@@H]2[C@H]1C(=O)NN(C[C@@H]1CCNC1=O)C(=O)[C@H](F)Cl]

### Step 1: Preparation of tert-butyl N-[[(3S,3aS,6aR)-2-[(2S)-2-[(6-fluoropyrimidin-4-yl)amino]-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate

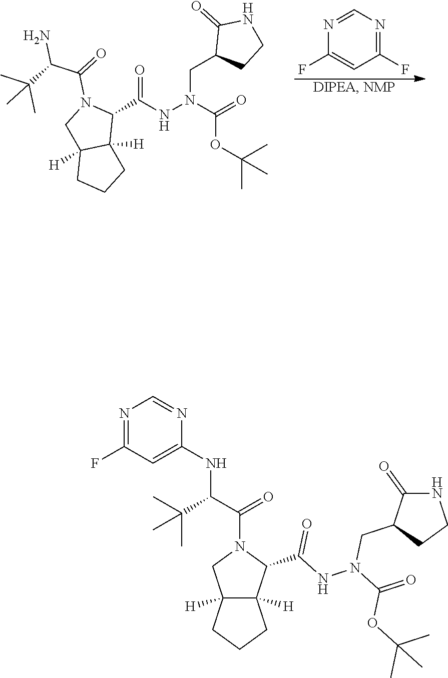

To a solution of tert-butyl N-[[(3S,3aS,6aR)-2-[(2S)-2-amino-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate (1.0 g, 2.09 mmol, from Example 29, step 4) in NMP (10 mL) was added DIPEA (539 mg, 4.17 mmol) and 4,6-difluoropyrimidine (242 mg, 2.09 mmol). The mixture was stirred at 50° C. for 2 h. The mixture was purified by prep-HPLC (Instrument GX-R, Method Column: Waters Xbridge 150*25 mm*5 um; Condition water (0.1% TFA)-CAN, Begin B 34, End B 64; Gradient Time(min) 8; 100% B Hold Time(min) 2; FlowRate (mL/min) 25; Injections 1) to afford tert-butyl N-[[(3S,3aS,6aR)-2-[(2S)-2-[(6-fluoropyrimidin-4-yl)amino]-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate; 2,2,2-trifluoroacetic acid (1.1 g) as a white solid.

MS obsd. (ESI⁺) [(M+H)⁺]: 576.4.

### Step 2: Preparation of (3S,3aS,6aR)-2-[(2S)-2-[(6-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide

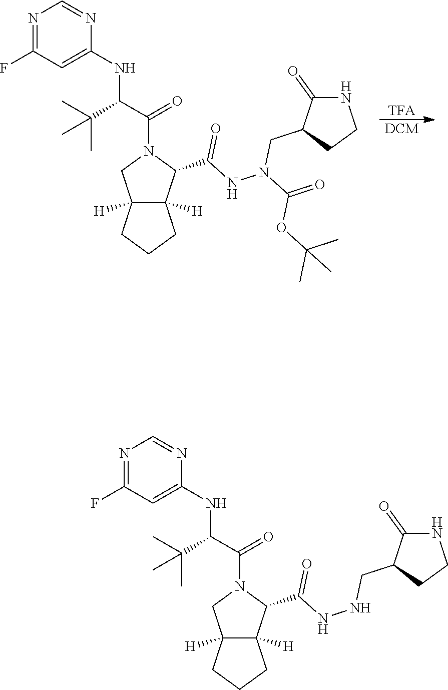

To a solution of tert-butyl N-[[(3S,3aS,6aR)-2-[(2S)-2-[(6-fluoropyrimidin-4-yl)amino]-3,3-dimethyl-butanoyl]-3,3a,4,5,6,6a-hexahydro-1H-cyclopenta[c]pyrrole-3-carbonyl]amino]-N-[[(3S)-2-oxopyrrolidin-3-yl]methyl]carbamate; 2,2,2-trifluoroacetic acid (500 mg, 0.72 mmol) in DCM (5 mL) was added TFA (2.0 mL). The mixture was stirred at 25° C. for 2 h. The mixture was concentrated in vacuum and the residue was purified by prep-HPLC (Instrument GX-R, Method Column: Waters Xbridge 150*25 mm*5 um; Condition: water (0.1% HCl)-ACN, Begin B: 34, End 64. Gradient Time (min) 8; 100% B Hold Time (min) 2; Flowrate (mL/min) 25; Injections: 25) to afford (3S,3aS,6aR)-2-[(2S)-2-[(6-fluoropyrimidin-4-yl)amino]-3,3-dimethyl-butanoyl]-N-[[(3S)-2-oxopyrrolidin-3-yl]methyl]-3,3a,4,5,6,6a-hexahydro-1H-cyclopenta[c]pyrrole-3-carbohydrazide; hydrochloride (350 mg) as yellow oil.

MS obsd. (ESI⁺) [(M+H)⁺]: 476.3.

### Step 3: Preparation of (3S,3aS,6aR)—N′-[(2R)-2-chloro-2-fluoro-acetyl]-2-[(2S)-2-[(6-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide

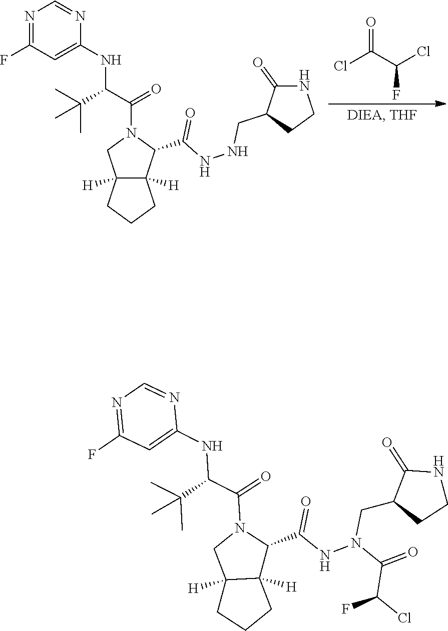

To a solution of (3S,3aS,6aR)-2-[(2S)-2-[(6-fluoropyrimidin-4-yl)amino]-3,3-dimethyl-butanoyl]-N-[[(3S)-2-oxopyrrolidin-3-yl]methyl]-3,3a,4,5,6,6a-hexahydro-1H-cyclopenta[c]pyrrole-3-carbohydrazide; hydrochloride (280 mg, 0.55 mmol) in THF (150 mL) was added DIPEA (711 mg, 5.5 mmol). After cooling to −5° C., to the mixture was added (2R)-2-chloro-2-fluoro-acetyl chloride (537.0 mg, 1.64 mmol, 40% purity, Intermediate 10) in THF (10 mL). The reaction mixture was stirred at −5° C. for 30 min. The reaction was quenched with MeOH (10 mL) and acidified by 4 N HCl/dioxane to pH=5 at −10° C. The resulting mixture was concentrated in vacuum at 30° C. The residue was purified by reversed flash chromatography (condition: 120 g Flash Column Welch Ultimate XB_C18 20-40 μm; 120 A, water (0.1% FA)-ACN, 0˜58%, 70 mL/min) to afford (3S,3aS,6aR)—N′-[(2R)-2-chloro-2-fluoro-acetyl]-2-[(2S)-2-[(6-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide (Example 182) (134 mg) as a white solid.

¹H NMR (400 MHz, DMSO-d₆) δ: ppm 11.06 (s, 1H), 8.28 (d, J=2.4 Hz, 1H), 8.23 (d, J=4.0 Hz, 1H), 7.77-7.70 (m, 1H), 7.20 (d, J=8.4 Hz, 1H), 7.03 (d, J=50.4 Hz, 1H), 4.66 (d, J=8.0 Hz, 1H), 4.08-4.02 (m, 1H), 3.93-3.85 (m, 2H), 3.72 (dd, J=13.6 Hz, 10.0 Hz, 1H), 3.42-3.37 (m, 2H), 3.21-3.10 (m, 2H), 2.81-2.69 (m, 1H), 2.61-2.52 (m, 1H), 2.19-2.09 (m, 1H), 1.93-1.89 (m, 1H), 1.80-1.58 (m, 5H), 1.49-1.32 (m, 1H), 1.04 (s, 9H).

MS obsd. (ESI⁺) [(M+H)⁺]: 570.4.

### Example 183

### (3S,3aS,6aR)—N′-[(2R)-2-chloro-2-fluoro-acetyl]-2-[(2S)-2-[(5-fluoropyrimidin-4-yl)amino]-3,3-dimethyl-butanoyl]-N-[[(3S)-2-oxopyrrolidin-3-yl]methyl]-3,3a,4,5,6,6a-hexahydro-1H-cyclopenta[c]pyrrole-3-carbohydrazide

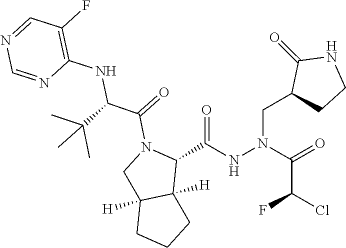 [SMILES: CC(C)(C)[C@H](Nc1ncncc1F)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 182, by using 4-chloro-5-fluoropyrimidine instead of 4,6-difluoropyrimidine to afford (3S,3aS,6aR)—N′-[(2R)-2-chloro-2-fluoro-acetyl]-2-[(2S)-2-[(5-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide (Example 183) as a white solid.

¹H NMR (400 MHz, DMSO-d₆) δ: ppm 11.06 (s, 1H), 8.28 (d, J=2.4 Hz, 1H), 8.23 (d, J=4.0 Hz, 1H), 7.77-7.70 (m, 1H), 7.20 (d, J=8.4 Hz, 1H), 7.03 (d, J=50.4 Hz, 1H), 4.66 (d, J=8.0 Hz, 1H), 4.08-4.02 (m, 1H), 3.93-3.85 (m, 2H), 3.72 (dd, J=13.6 Hz, 10.0 Hz, 1H), 3.42-3.37 (m, 2H), 3.21-3.10 (m, 2H), 2.81-2.69 (m, 1H), 2.61-2.52 (m, 1H), 2.19-2.09 (m, 1H), 1.93-1.89 (m, 1H), 1.80-1.58 (m, 5H), 1.49-1.32 (m, 1H), 1.04 (s, 9H).

MS obsd. (ESI⁺) [(M+H)⁺]: 570.4.

### Example 184

### (3S,3aS,6aR)—N-[(2R)-2-chloro-2-fluoro-acetyl]-2-[(2S)-3,3-dimethyl-2-(2-pyridylamino)butanoyl]-N′—[[(3S)-2-oxopyrrolidin-3-yl]methyl]-3,3a,4,5,6,6a-hexahydro-1H-cyclopenta[c]pyrrole-3-carbohydrazide

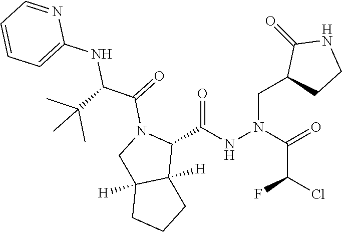 [SMILES: CC(C)(C)[C@H](Nc1ccccn1)C(=O)N1C[C@@H]2CCC[C@@H]2[C@H]1C(=O)NN(C[C@@H]1CCNC1=O)C(=O)[C@H](F)Cl]

The title compound was prepared in analogy to the procedure described for the preparation of Example 182, by using pyridine-N-oxide instead of 4,6-difluoropyrimidine in the presence of PyBop to afford (3S,3aS,6aR)—N-[(2R)-2-chloro-2-fluoro-acetyl]-2-[(2S)-3,3-dimethyl-2-(2-pyridylamino)butanoyl]-N′—[[(3S)-2-oxopyrrolidin-3-yl]methyl]-3,3a,4,5,6,6a-hexahydro-1H-cyclopenta[c]pyrrole-3-carbohydrazide (Example 184) as a white solid.

¹H NMR (400 MHz, DMSO-d₆) δ: ppm 11.01 (s, 1H) 7.88 (d, J=4.0 Hz, 1H), 7.77 (s, 1H), 7.38-7.32 (m, 1H), 7.07 (d, J=50.4 Hz, 1H), 6.77 (d, J=8.0 Hz, 1H), 6.58 (d, J=9.2 Hz, 1H), 6.48 (t, J=6.0 Hz, 1H), 4.49 (d, J=8.8 Hz, 1H), 4.24 (d, J=10.0 Hz, 1H), 3.88-3.84 (m, 2H), 3.70 (dd, J=10.4 Hz, 9.6 Hz, 1H), 3.40 (dd, J=13.2 Hz, 3.6 Hz, 1H), 3.21-3.10 (m, 2H), 2.77-2.70 (m, 1H), 2.60-2.54 (m, 2H), 2.20-2.08 (m, 1H), 1.97-1.87 (m, 1H), 1.81-1.62 (m, 5H), 1.45-1.37 (m, 1H), 1.01 (s, 9H).

MS obsd. (ESI⁺) [(M+H)⁺]: 551.3.

### Example 185

### (2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide

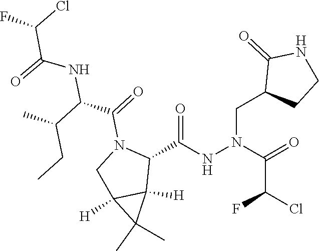 [SMILES: CC[C@H](C)[C@H](NC(=O)[C@H](F)Cl)C(=O)N1C[C@H]2[C@@H]([C@H]1C(=O)NN(C[C@@H]1CCNC1=O)C(=O)[C@H](F)Cl)C2(C)C]

### Step 1: Preparation of (1R,2S,5S)-3-[(2S,3S)-2-amino-3-methyl-pentanoyl]-6,6-dimethyl-N′—[[(3S)-2-oxopyrrolidin-3-yl]methyl]-3-azabicyclo[3.1.0]hexane-2-carbohydrazide

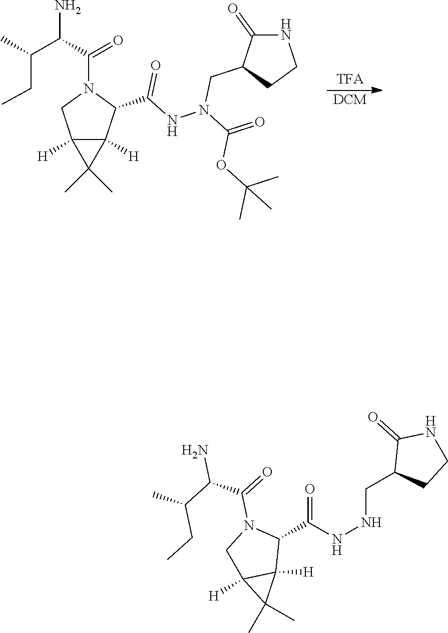

To a solution of tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[[(3S)-2-oxopyrrolidin-3-yl]methyl]carbamate (450 mg, 0.94 mmol, from Example 105, Step 4) in DCM (30 mL) was added TFA (2.0 mL). The mixture was stirred at 25° C. for 1 h and concentrated in vacuum. The residue was purified by reverse flash chromatography (condition: 120 g Flash Column Welch Ultimate XB_C18 20-40 μm; 120 A, water (0.1% HCl)-ACN, 0˜40%, 70 mL/min) to afford (1R,2S,5S)-3-[(2S,3S)-2-amino-3-methyl-pentanoyl]-6,6-dimethyl-N′—[[(3S)-2-oxopyrrolidin-3-yl]methyl]-3-azabicyclo[3.1.0]hexane-2-carbohydrazide; dihydrochloride (420 mg) as a white solid.

MS obsd. (ESI⁺) [(M+H)⁺]: 380.3.

### Step 2: Preparation of (2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide

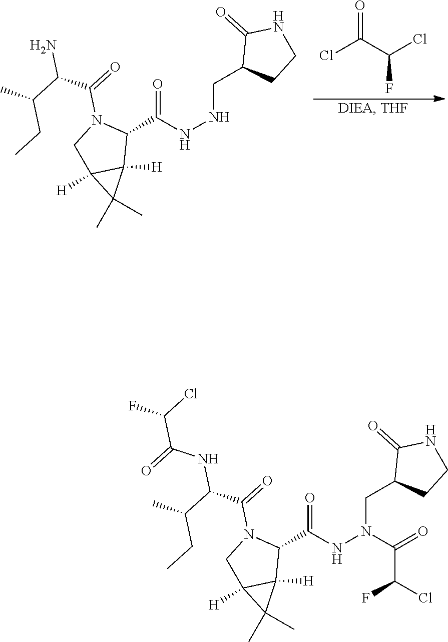

To a solution of (1R,2S,5S)-3-[(2S,3S)-2-amino-3-methyl-pentanoyl]-6,6-dimethyl-N-[[(3S)-2-oxopyrrolidin-3-yl]methyl]-3-azabicyclo[3.1.0]hexane-2-carbohydrazide; dihydrochloride (350 mg, 0.77 mmol) in THF (100 mL) was added DIPEA (1500 mg, 11.6 mmol). After cooling to −5° C., to the mixture was added (2R)-2-chloro-2-fluoro-acetyl chloride (933 mg, 2.71 mmol). The reaction mixture was stirred at −5° C. for 30 min. The reaction was quenched with 2 mL MeOH at 0° C., then acidified by 4 N HCl/dioxane until pH=4. The mixture was concentrated in vacuum and the residue was purified by reversed flash chromatography (condition: 40 g Flash Column Welch Ultimate XB_C18 20-40 μm; 120 A, water (0.1% FA)-ACN, 0˜45%, 70 mL/min) to afford (2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide (16.8 mg, Example 185) as a white solid.

¹H NMR (400 MHz, DMSO-d₆) δ: ppm 11.16 (s, 1H), 9.11-9.09 (m, 1H), 7.76 (s, 1H), 6.78 (m, 2H), 4.24-4.18 (m, 1H), 4.15 (s, 1H), 3.94-3.85 (m, 2H), 3.80 (dd, J=14.4 Hz, 9.6 Hz, 1H), 3.45 (dd, J=14.0 Hz, 4.4 Hz, 1H), 3.21-3.13 (m, 2H), 2.63-2.56 (m, 1H), 2.20-2.11 (m, 1H), 1.87-1.73 (m, 2H), 1.68-1.61 (m, 1H), 1.56-1.53 (m, 1H), 1.51-1.41 (m, 1H), 1.16-1.08 (m, 1H), 1.06 (s, 3H), 0.92 (s, 3H), 0.86 (d, J=6.4 Hz, 3H), 0.81 (t, J=7.2 Hz, 3H).

MS obsd. (ESI⁺) [(M+H)⁺]: 568.2.

### Example 186

### (2S)-2-chloro-N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide

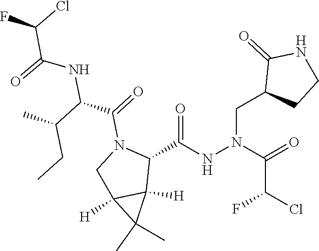 [SMILES: CC[C@H](C)[C@H](NC(=O)[C@@H](F)Cl)C(=O)N1C[C@H]2[C@@H]([C@H]1C(=O)NN(C[C@@H]1CCNC1=O)C(=O)[C@@H](F)Cl)C2(C)C]

The title compound was prepared in analogy to the procedure described for the preparation of Example 185, by using (2S)-2-chloro-2-fluoro-acetyl chloride (Intermediate 28) instead of (2R)-2-chloro-2-fluoro-acetyl chloride (Intermediate 10) to afford (2S)-2-chloro-N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide (Example 186) as a white solid.

¹H NMR (400 MHz, DMSO-d₆) δ: ppm 11.13-10.83 (m, 1H), 9.09-9.04 (m, 1H), 7.75-7.33 (m, 1H), 7.17-6.57 (m, 2H), 4.26-4.13 (m, 2H), 3.90-3.82 (m, 2H), 3.77-3.51 (m, 2H), 3.20-2.96 (m, 2H), 2.64-2.53 (m, 1H), 2.21-2.12 (m, 1H), 1.88-1.73 (m, 2H), 1.66-1.45 (m, 3H), 1.14-1.05 (m, 4H), 0.90-0.80 (m, 9H).

MS obsd. (ESI⁺) [(M+H)⁺]: 568.3.

### Example 187

### (2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide

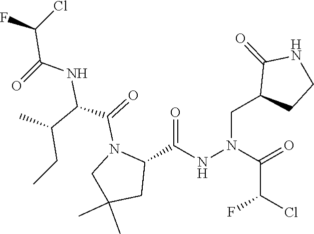 [SMILES: CC[C@H](C)[C@H](NC(=O)[C@@H](F)Cl)C(=O)N1CC(C)(C)C[C@H]1C(=O)NN(C[C@@H]1CCNC1=O)C(=O)[C@@H](F)Cl]

### Step 1: Preparation of methyl (2S)-1-[(2S,3S)-2-(tert-butoxycarbonylamino)-3-methyl-pentanoyl]-4,4-dimethyl-pyrrolidine-2-carboxylate

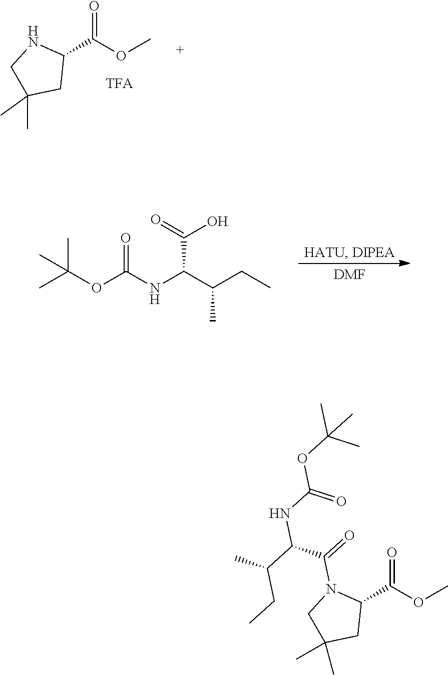

To a solution of Boc-Ile-OH (474 mg, 2.05 mmol) in DMF (10 mL) was added DIPEA (1589 mg, 12.3 mmol), HATU (974 mg, 2.56 mmol) and methyl (2S)-4,4-dimethylpyrrolidine-2-carboxylate; 2,2,2-trifluoroacetic acid (556 mg, 2.05 mmol, Intermediate 43) at 0° C. The mixture was stirred at 25° C. for 12 h. The reaction was diluted with H₂O (50 mL) and extracted with EtOAc (50 mL×3). The combined organic layers were washed with brine (50 mL), dried over Na₂SO₄, filtered and concentrated in vacuum. The residue was purified by silica gel column eluted with PE/EtOAc=4/1 to afford methyl (2S)-1-[(2S,3S)-2-(tert-butoxycarbonylamino)-3-methyl-pentanoyl]-4,4-dimethyl-pyrrolidine-2-carboxylate (680 mg) as yellow oil.

MS obsd. (ESI⁺) [(M+H)⁺]: 371.3.

### Step 2: Preparation of (2S)-1-[(2S,3S)-2-(tert-butoxycarbonylamino)-3-methyl-pentanoyl]-4,4-dimethyl-pyrrolidine-2-carboxylic acid

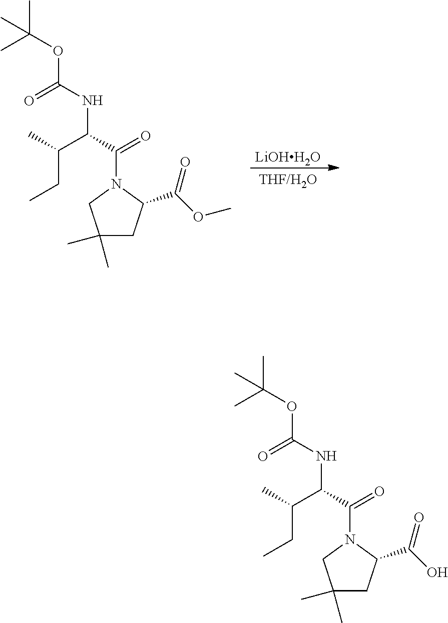 [SMILES: CC[C@H](C)[C@H](NC(=O)OC(C)(C)C)C(=O)N1CC(C)(C)C[C@H]1C(=O)OC ; "LiOH·H2O" ; "THF/H2O" ; CC[C@H](C)[C@H](NC(=O)OC(C)(C)C)C(=O)N1CC(C)(C)C[C@H]1C(=O)O]

To a solution of methyl (2S)-1-[(2S,3S)-2-(tert-butoxycarbonylamino)-3-methyl-pentanoyl]-4,4-dimethyl-pyrrolidine-2-carboxylate (680 mg, 1.84 mmol) in THF (6 mL) and water (6 mL) was added LiOH·H₂O (154 mg, 3.67 mmol) at 0° C. The mixture was stirred at 25° C. for 2 h. The reaction was diluted with 40 mL water and acidified with 1 N HCl to pH=4. The resulting mixture was extracted with EtOAc (50 mL×2). The organic layers were washed with brine (60 mL), dried over Na₂SO₄, filtered and concentrated in vacuum to afford (2S)-1-[(2S,3S)-2-(tert-butoxycarbonylamino)-3-methyl-pentanoyl]-4,4-dimethyl-pyrrolidine-2-carboxylic acid (650 mg) as a white solid.

MS obsd. (ESI⁺) [(M+H)⁺]: 357.2.

### Step 3: Preparation of tert-butyl N-[[(2S)-1-[(2S,3S)-2-(tert-butoxycarbonylamino)-3-methyl-pentanoyl]-4,4-dimethyl-pyrrolidine-2-carbonyl]amino]-N-[[(3S)-2-oxopyrrolidin-3-yl]methyl]carbamate

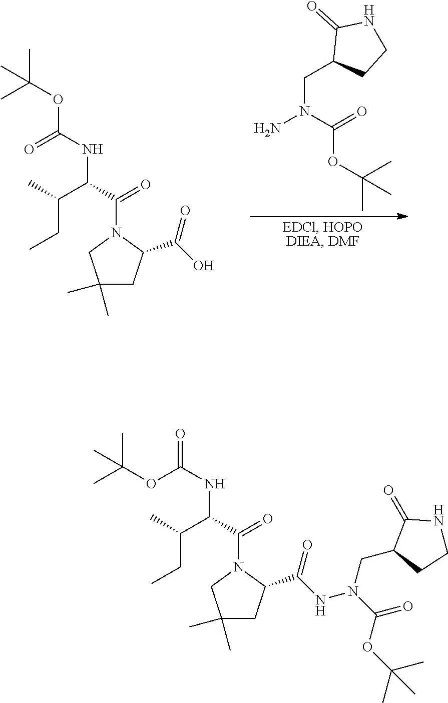

To a solution of (2S)-1-[(2S,3S)-2-(tert-butoxycarbonylamino)-3-methyl-pentanoyl]-4,4-dimethyl-pyrrolidine-2-carboxylic acid (620 mg, 1.74 mmol) in DMF (10 mL) was added DIPEA (562 mg, 4.35 mmol), EDCI (433 mg, 2.26 mmol), HOPO (251 mg, 2.26 mmol) and tert-butyl N-amino-N-[[(3S)-2-oxopyrrolidin-3-yl]methyl]carbamate (399 mg, 1.74 mmol). The mixture was stirred at 25° C. for 12 h. The reaction mixture was diluted with EtOAc (100 mL), washed with 1 N HCl (30 mL), brine (40 mL), dried over Na₂SO₄ and concentrated in vacuum to afford tert-butyl N-[[(2S)-1-[(2S,3S)-2-(tert-butoxycarbonylamino)-3-methyl-pentanoyl]-4,4-dimethyl-pyrrolidine-2-carbonyl]amino]-N-[[(3S)-2-oxopyrrolidin-3-yl]methyl]carbamate (913 mg) was obtained as a white solid.

MS obsd. (ESI⁺) [(M+H)⁺]: 568.4.

### Step 4: Preparation of (2S)-1-[(2S,3S)-2-amino-3-methyl-pentanoyl]-4,4-dimethyl-N′—[[(3S)-2-oxopyrrolidin-3-yl]methyl]pyrrolidine-2-carbohydrazide

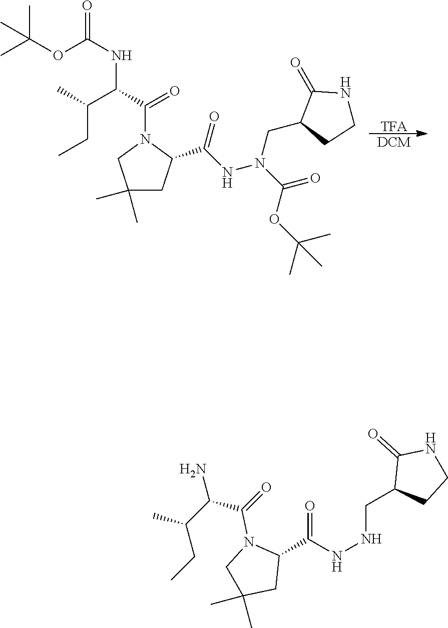

To a solution of tert-butyl N-[[(2S)-1-[(2S,3S)-2-(tert-butoxycarbonylamino)-3-methyl-pentanoyl]-4,4-dimethyl-pyrrolidine-2-carbonyl]amino]-N-[[(3S)-2-oxopyrrolidin-3-yl]methyl]carbamate (913 mg, 1.61 mmol) in DCM (10 mL) was added TFA (5.0 mL). The mixture was stirred at 25° C. for 1 h and concentrated in vacuum. The residue was purified by reverse flash chromatography (condition: 120 g Flash Column Welch Ultimate XB_C18 20-40 μm; 120 A, water (0.1% NH₃)-ACN, 45%, 45 mL/min) to afford (2S)-1-[(2S,3S)-2-amino-3-methyl-pentanoyl]-4,4-dimethyl-N-[[(3S)-2-oxopyrrolidin-3-yl]methyl]pyrrolidine-2-carbohydrazide (640 mg) as a white solid.

MS obsd. (ESI⁺) [(M+H)⁺]: 368.4.

### Step 5: Preparation of (2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide

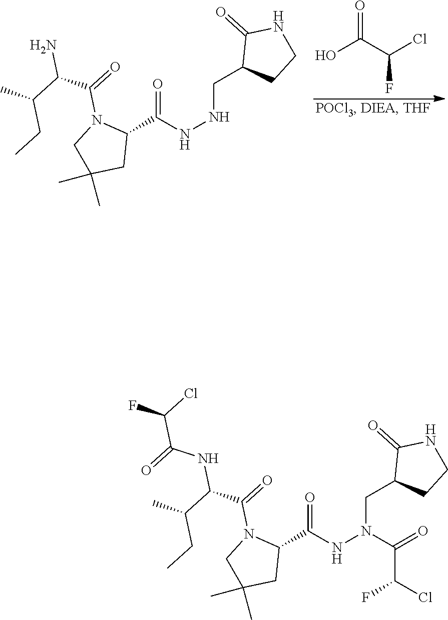

To a solution of (2S)-2-chloro-2-fluoro-acetic acid (1266 mg, 7.31 mmol, 62% purity) in DCM (40 mL) was added POCl₃ (1041.35 mg, 6.79 mmol). The mixture was stirred at 25° C. for 1 h. The mixture was added to a solution of (2S)-1-[(2S,3S)-2-amino-3-methyl-pentanoyl]-4,4-dimethyl-N-[[(3S)-2-oxopyrrolidin-3-yl]methyl]pyrrolidine-2-carbohydrazide (640 mg, 1.74 mmol) and DIPEA (4502 mg, 34.83 mmol) in DCM (40 mL) at −5° C. dropwise. The resulting mixture was stirred for 1 h at −5° C. The reaction mixture was quenched with MeOH (10 mL) and acidified with 4 N HCl/dioxane to pH=5 at −10° C. The resulting mixture was concentrated in vacuum at 30° C. The residue was purified by reversed flash chromatography (condition: 40 g Flash Column Welch Ultimate XB_C18 20-40 μm; 120 A, water (0.1% FA)-ACN, 59%, 45 mL/min) to afford (2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide (60 mg, Example 187) as a white solid.

¹H NMR (400 MHz, DMSO-d₆) δ: ppm 11.02-10.73 (m, 1H), 9.07 (dd, J=23.2 Hz, 8.4 Hz, 1H), 7.74-7.73 (m, 1H), 7.30-6.42 (m, 2H), 4.37-4.25 (m, 2H), 3.78-3.65 (m, 2H), 3.58-3.51 (m, 1H), 3.32-3.10 (m, 1H), 3.20-2.98 (m, 2H), 2.82-2.62 (m, 1H), 2.26-2.08 (m, 1H), 1.99-1.92 (m, 1H), 1.87-1.67 (m, 3H), 1.54-1.45 (m, 1H), 1.16 (s, 3H), 1.13-1.07 (m, 1H), 1.00 (s, 3H), 0.89 (d, J=6.4 Hz, 3H), 0.82 (t, J=7.6 Hz, 3H).

MS obsd. (ESI⁺) [(M+H)⁺]: 556.1.

### Example 188

### (2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide

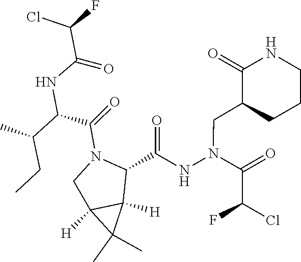 [SMILES: CC[C@H](C)[C@H](NC(=O)[C@H](F)Cl)C(=O)N1C[C@H]2[C@@H]([C@H]1C(=O)NN(C[C@@H]1CCCNC1=O)C(=O)[C@H](F)Cl)C2(C)C]

### Step 1: Preparation of methyl (2S)-1-[(2S,3S)-2-(tert-butoxycarbonylamino)-3-methyl-pentanoyl]-4,4-dimethyl-pyrrolidine-2-carboxylate

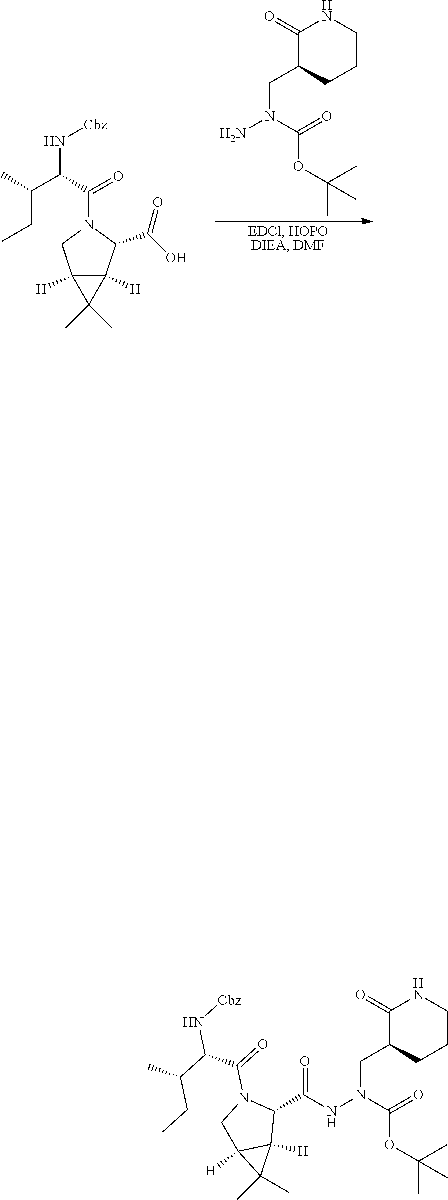

To a solution of (1R,2S,5S)-3-[(2S,3S)-2-(benzyloxycarbonylamino)-3-methyl-pentanoyl]-6,6-dimethyl-3-azabicyclo[3.1.0]hexane-2-carboxylic acid (413 mg, 1.03 mmol, from Example 105, step 2) in DMF (5 mL) was added DIEA (398 mg, 3.08 mmol), EDCI (236 mg, 1.23 mmol), HOPO (137 mg, 1.23 mmol) and tert-butyl N-amino-N-[[(3S)-2-oxo-3-piperidyl]methyl]carbamate (250 mg, 1.03 mmol, Intermediate 39a). The mixture was stirred at 25° C. and 16 h. Then the reaction mixture was poured into water (100 mL) and extracted with EtOAc (60 mL×2). The organic layers were washed with 1N HCl(60 mL), 1N NaOH (50 mL) and brine (60 mL×2), dried over Na₂SO₄ and concentrated in vacuum to afford tert-butyl N-[[(1R,2S,5S)-3-[(2S,3S)-2-(benzyloxycarbonylamino)-3-methyl-pentanoyl]-6,6-dimethyl-3-azabicyclo[3.1.0]hexane-2-carbonyl]amino]-N-[[(3S)-2-oxo-3-piperidyl]methyl]carbamate (510 mg) as a yellow foam.

MS obsd. (ESI⁺) [(M+H)⁺]: 628.4.

### Step 2: Preparation of (1R,2S,5S)-3-[(2S,3S)-2-amino-3-methyl-pentanoyl]-6,6-dimethyl-N′—[[(3S)-2-oxo-3-piperidyl]methyl]-3-azabicyclo[3.1.0]hexane-2-carbohydrazide

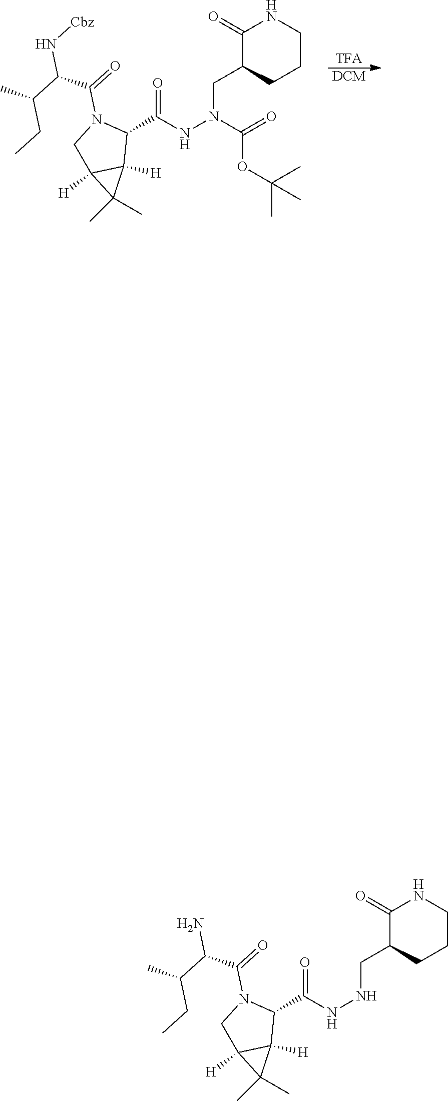

To a solution of tert-butyl N-[[(1R,2S,5S)-3-[(2S,3S)-2-amino-3-methyl-pentanoyl]-6,6-dimethyl-3-azabicyclo[3.1.0]hexane-2-carbonyl]amino]-N-[[(3S)-2-oxo-3-piperidyl]methyl]carbamate (280 mg, 0.57 mmol) in DCM (10 mL) was added TFA (5.0 mL). The mixture was stirred at 25° C. for 1 h. The resulting mixture was concentrated in vacuum and the residue was purified by reverse flash (120 g Flash Column; Welch Ultimate XB_C18 20-40 μm; 35 min; 75 mL/min, ACN-Water, 0.1% NH₃·H₂O) to afford (1R,2S,5S)-3-[(2S,3S)-2-amino-3-methyl-pentanoyl]-6,6-dimethyl-N-[[(3S)-2-oxo-3-piperidyl]methyl]-3-azabicyclo[3.1.0]hexane-2-carbohydrazide (135 mg) as a white solid.

MS obsd. (ESI⁺) [(M+H)⁺]: 394.3.

### Step 3: Preparation of (2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide

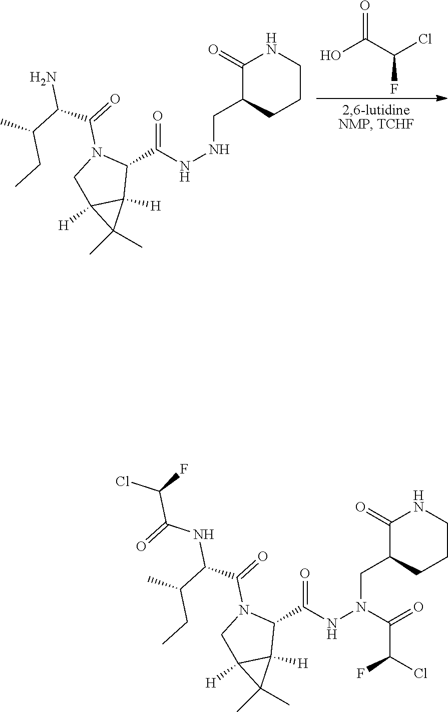

To a solution of (1R,2S,5S)-3-[(2S,3S)-2-amino-3-methyl-pentanoyl]-6,6-dimethyl-NV-[[(3S)-2-oxo-3-piperidyl]methyl]-3-azabicyclo[3.1.0]hexane-2-carbohydrazide (590 mg, 1.5 mmol), (2R)-2-chloro-2-fluoro-acetic acid (816 mg, 4.5 mmol,) and 2,6-lutidine (1.4 mL, 11.99 mmol) in NMP (20 mL) was added TCFH (1052 mg, 3.75 mmol). The suspension was stirred at 25° C. for 30 min. The resulting mixture was poured into 1N HCl (100 mL) and extracted with EtOAc (50 mL×2). The organic layers were washed with brine (100 mL), dried over Na₂SO₄ and concentrated in vacuum. The residue was purified by reverse flash (120 g Flash Column; Welch Ultimate XB_C18 20-40 μm; 55 min; 0˜60%, 65 mL/min, ACN-Water, 0.1% FA) to afford (2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide (30 mg, Example 188) as a white solid.

¹H NMR (400 MHz, DMSO-d6) δ: ppm 11.14 (s, 1H), 9.10 (d, J=8.0 Hz, 1H), 7.52 (s, 1H), 6.93-6.61 (m, 2H), 4.22 (t, J=8.4 Hz, 1H), 4.14 (s, 1H), 4.04-3.93 (m, 1H), 3.87 (s, 2H), 3.44 (dd, J=14.0 Hz, 4.0 Hz, 1H), 3.11 (s, 2H), 2.48-2.40 (m, 1H), 1.89-1.73 (m, 3H), 1.70-1.55 (m, 2H), 1.54-1.48 (m, 1H), 1.45 (d, J=7.2 Hz, 2H), 1.15-1.07 (m, 1H), 1.05 (s, 3H), 0.89 (s, 3H), 0.85 (d, J=6.8 Hz, 3H), 0.80 (t, J=7.6 Hz, 3H).

MS obsd. (ESI⁺) [(M+H)⁺]: 582.3.

### Example 189

### (2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide

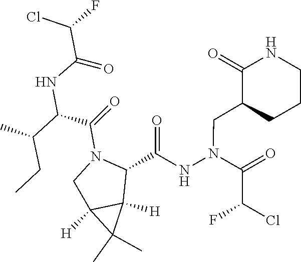 [SMILES: CC[C@H](C)[C@H](NC(=O)[C@@H](F)Cl)C(=O)N1C[C@H]2[C@@H]([C@H]1C(=O)NN(C[C@@H]1CCCNC1=O)C(=O)[C@@H](F)Cl)C2(C)C]

The title compound was prepared in analogy to the procedure described for the preparation of Example 188, by using (2S)-2-chloro-2-fluoro-acetic acid instead of (2R)-2-chloro-2-fluoro-acetic acid to afford (2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide (Example 189) as a white solid.

¹H NMR (400 MHz, DMSO-d6) δ: ppm 10.95 (d, J=99.2 Hz, 1H), 9.12-9.06 (m, 1H), 7.51 (d, J=31.2 Hz, 1H), 7.20-6.45 (m, 2H), 4.26-4.11 (m, 2H), 3.95-3.83 (m, 2H), 3.79-3.69 (m, 1H), 3.62-3.48 (m, 1H), 3.15-3.03 (m, 2H), 2.61-2.56 (m, 1H), 2.05-1.65 (m, 3H), 1.63-1.44 (m, 4H), 1.43-1.23 (m, 1H), 1.17-1.09 (m, 1H), 1.06 (s, 3H), 0.90 (s, 3H), 0.87 (d, J=6.8 Hz, 3H), 0.82 (d, J=7.2 Hz, 3H).

MS obsd. (ESI⁺) [(M+H)⁺]: 582.1.

### Example 190

### (2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide

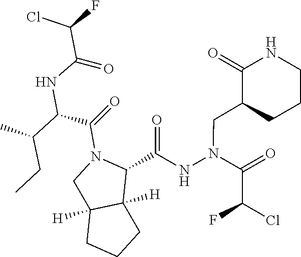 [SMILES: CC[C@H](C)[C@H](NC(=O)[C@H](F)Cl)C(=O)N1C[C@@H]2CCC[C@@H]2[C@H]1C(=O)NN(C[C@@H]1CCCNC1=O)C(=O)[C@H](F)Cl]

The title compound was prepared in analogy to the procedure described for the preparation of Example 187, by using ethyl (3S,3aS,6aR)-1,2,3,3a,4,5,6,6a-octahydrocyclopenta[c]pyrrole-3-carboxylate; hydrochloride instead of methyl (2S)-4,4-dimethylpyrrolidine-2-carboxylate; 2,2,2-trifluoroacetic acid (Intermediate 43), (2R)-2-chloro-2-fluoro-acetic acid instead of (2S)-2-chloro-2-fluoro-acetic acid, and tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9) to afford (2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide (Example 190) as a white solid.

¹H NMR (400 MHz, DMSO-d6) δ: ppm 11.07 (s, 1H), 9.08 (d, J=8.0 Hz, 1H), 7.57 (s, 1H), 6.95 (d, J=50.8 Hz, 1H), 6.73 (d, J=49.6 Hz, 1H), 4.32 (t, J=8.4 Hz, 1H), 4.02-3.92 (m, 2H), 3.90-3.74 (m, 2H), 3.40-3.35 (m, 1H), 3.15-3.07 (m, 2H), 2.82-2.71 (m, 1H), 2.58-2.54 (m, 1H), 2.43-2.36 (m, 1H), 1.93-1.71 (m, 7H), 1.67-1.54 (m, 2H), 1.52-1.36 (m, 3H), 1.17-1.04 (m, 1H), 0.87 (d, J=6.8 Hz, 3H), 0.81 (t, J=7.2 Hz, 3H).

MS obsd. (ESI⁺) [(M+H)⁺]:582.0.

### Example 191

### (2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide

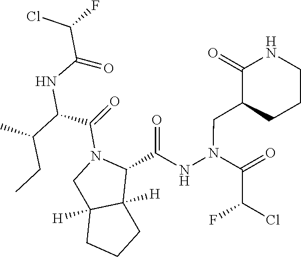 [SMILES: CC[C@H](C)[C@H](NC(=O)[C@@H](F)Cl)C(=O)N1C[C@@H]2CCC[C@@H]2[C@H]1C(=O)NN(C[C@@H]1CCCNC1=O)C(=O)[C@@H](F)Cl]

The title compound was prepared in analogy to the procedure described for the preparation of Example 187, by using ethyl (3S,3aS,6aR)-1,2,3,3a,4,5,6,6a-octahydrocyclopenta[c]pyrrole-3-carboxylate; hydrochloride instead of methyl (2S)-4,4-dimethylpyrrolidine-2-carboxylate; 2,2,2-trifluoroacetic acid (Intermediate 43), and tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9) to afford (2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide (Example 191) as a white solid.

¹H NMR (400 MHz, DMSO-d₆) δ: ppm 10.9 (d, J=119.2 Hz, 1H), 9.09-9.00 (m, 1H), 7.53 (d, J=28.8 Hz, 1H), 7.20-6.35 (m, 2H), 4.38-4.28 (m, 1H), 4.02 (s, 1H), 3.95-3.45 (m, 4H), 3.10 (s, 2H), 2.78-2.71 (m, 1H), 2.64-2.55 (m, 1H), 2.47-2.36 (m, 1H), 2.10-1.80 (m, 4H), 1.79-1.61 (m, 4H), 1.59-1.32 (m, 4H), 1.17-1.03 (m, 1H), 0.90-0.85 (m, 3H), 0.82 (t, J=7.2 Hz, 3H).

MS obsd. (ESI⁺) [(M+H)⁺]:582.1.

### Example 192

### (2R)—N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide

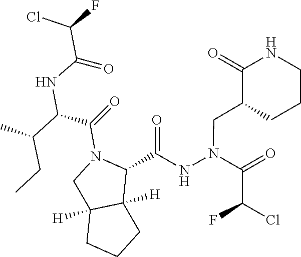 [SMILES: CC[C@H](C)[C@H](NC(=O)[C@H](F)Cl)C(=O)N1C[C@@H]2CCC[C@@H]2[C@H]1C(=O)NN(C[C@H]1CCCNC1=O)C(=O)[C@H](F)Cl]

The title compound was prepared in analogy to the procedure described for the preparation of Example 187, by using ethyl (3S,3aS,6aR)-1,2,3,3a,4,5,6,6a-octahydrocyclopenta[c]pyrrole-3-carboxylate; hydrochloride instead of methyl (2S)-4,4-dimethylpyrrolidine-2-carboxylate; 2,2,2-trifluoroacetic acid (Intermediate 43), (2R)-2-chloro-2-fluoro-acetic acid instead of (2S)-2-chloro-2-fluoro-acetic acid, and tert-butyl N-amino-N-[[(3R)-2-oxo-3-piperidyl]methyl]carbamate (Intermediate 39b) instead of tert-butyl N-amino-N-[[(3S)-2-oxopyrrolidin-3-yl]methyl]carbamate (Intermediate 9) to afford (2R)—N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide (Example 192) as a white solid.

¹H NMR (400 MHz, DMSO-d6) δ: ppm 10.98 (s, 1H), 9.08 (d, J=8.4 Hz, 1H), 7.49 (s, 1H), 6.93 (d, J=50.4 Hz, 1H), 6.73 (d, J=49.6 Hz, 1H), 4.31 (d, J=8.8 Hz, 1H), 4.00 (d, J=5.6 Hz, 1H), 3.95-3.73 (m, 3H), 3.58 (dd, J=13.6 Hz, 8.4 Hz, 1H), 3.21-3.06 (m, 2H), 2.80-2.70 (m, 1H), 2.65-2.56 (m, 1H), 2.36-2.23 (m, 1H), 2.00-1.69 (m, 7H), 1.66-1.35 (m, 5H), 1.18-1.02 (m, 1H), 0.87 (d, J=6.8 Hz, 3H), 0.81 (t, J=7.2 Hz, 3H).

MS obsd. (ESI⁺) [(M+H)⁺]:582.0.

### Example 193

### (2S)—N-[(1S,2S)-1-[(3S,3aS,6aR)-3-[[[(2S)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide

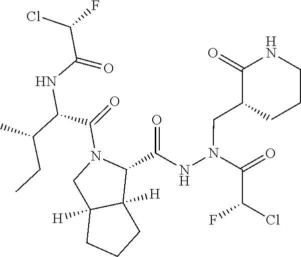 [SMILES: CC[C@H](C)[C@H](NC(=O)[C@@H](F)Cl)C(=O)N1C[C@@H]2CCC[C@@H]2[C@H]1C(=O)NN(C[C@H]1CCCNC1=O)C(=O)[C@@H](F)Cl]

The title compound was prepared in analogy to the procedure described for the preparation of Example 187, by using ethyl (3S,3aS,6aR)-1,2,3,3a,4,5,6,6a-octahydrocyclopenta[c]pyrrole-3-carboxylate; hydrochloride instead of methyl (2S)-4,4-dimethylpyrrolidine-2-carboxylate; 2,2,2-trifluoroacetic acid (Intermediate 43), and tert-butyl N-amino-N-[[(3R)-2-oxo-3-piperidyl]methyl]carbamate (Intermediate 39b) instead of tert-butyl N-amino-N-[[(3S)-2-oxopyrrolidin-3-yl]methyl]carbamate (Intermediate 9) to afford (2S)—N-[(1S,2S)-1-[(3S,3aS,6aR)-3-[[[(2S)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide (Example 193) as a white solid.

¹H NMR (400 MHz, DMSO-d6) δ: ppm 10.08 (d, J=132.0 Hz, 1H), 9.10-8.89 (m, 1H), 7.53 (d, J=22.8 Hz, 1H), 7.20-6.50 (m, 2H), 4.40-4.25 (m, 1H), 4.10-3.40 (m, 5H), 3.12 (s, 2H), 2.76 (s, 1H), 2.66-2.55 (m, 1H), 2.41-2.25 (m, 1H), 1.94-1.81 (m, 4H), 1.80-1.60 (m, 4H), 1.58-1.32 (m, 4H), 1.18-1.01 (m, 1H), 0.93-0.86 (m, 3H), 0.82 (t, J=7.2 Hz, 3H).

MS obsd. (ESI⁺) [(M+H)⁺]:582.1.

### Example 194

### (2R)-2-chloro-N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide

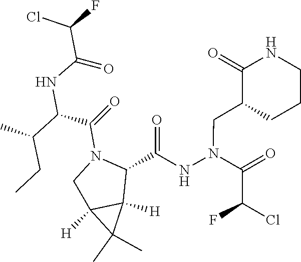 [SMILES: CC[C@H](C)[C@H](NC(=O)[C@H](F)Cl)C(=O)N1C[C@H]2[C@@H]([C@H]1C(=O)NN(C[C@H]1CCCNC1=O)C(=O)[C@H](F)Cl)C2(C)C]

The title compound was prepared in analogy to the procedure described for the preparation of Example 188, by using tert-butyl N-amino-N-[[(3R)-2-oxo-3-piperidyl]methyl]carbamate (Intermediate 39b) instead of tert-butyl N-amino-N-[[(3S)-2-oxo-3-piperidyl]methyl]carbamate (Intermediate 39a) to afford (2R)-2-chloro-N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide (Example 194) as a white solid.

¹H NMR (400 MHz, DMSO-d₆) δ: ppm 11.08 (s, 1H), 9.10 (d, J=8.0 Hz, 1H), 7.51 (s, 1H), 6.93-6.60 (m, 2H), 4.19 (d, J=8.8 Hz, 1H), 4.16 (s, 1H), 3.94-3.82 (m, 3H), 3.62 (dd, J=13.6, 8.4 Hz, 1H), 3.17-3.06 (m, 2H), 2.41-2.30 (m, 1H), 1.95-1.85 (m, 1H), 1.84-1.74 (m, 2H), 1.67-1.60 (m, 1H), 1.60-1.55 (m, 1H), 1.55-1.40 (m, 3H), 1.14-1.06 (m, 1H), 1.04 (s, 3H), 0.91 (s, 3H), 0.84 (d, J=6.8 Hz, 3H), 0.80 (t, J=7.6 Hz, 3H).

MS obsd. (ESI⁺) [(M+H)⁺]: 582.2.

### Example 195

### (2S)-2-chloro-N-[(1S,2S)-1-[(1R,2S,5S)-2-[[[(2S)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide

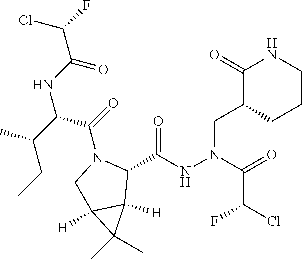 [SMILES: CC[C@H](C)[C@H](NC(=O)[C@@H](F)Cl)C(=O)N1C[C@H]2[C@@H]([C@H]1C(=O)NN(C[C@H]1CCCNC1=O)C(=O)[C@@H](F)Cl)C2(C)C]

The title compound was prepared in analogy to the procedure described for the preparation of Example 188, by using tert-butyl N-amino-N-[[(3R)-2-oxo-3-piperidyl]methyl]carbamate (Intermediate 39b) instead of tert-butyl N-amino-N-[[(3S)-2-oxo-3-piperidyl]methyl]carbamate (Intermediate 39a), and (2S)-2-chloro-2-fluoro-acetic acid instead of (2R)-2-chloro-2-fluoro-acetic acid to afford (2S)-2-chloro-N-[(1S,2S)-1-[(1R,2S,5S)-2-[[[(2S)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide (Example 195) as a white solid.

¹H NMR (400 MHz, DMSO-d6) δ: ppm 10.93 (d, J=104.8 Hz, 1H), 9.06 (dd, J=33.6 Hz, 7.2 Hz, 1H), 7.51 (s, 1H), 7.17-6.58 (m, 2H), 4.27-4.12 (m, 2H), 3.96-3.77 (m, 3H), 3.11 (s, 2H), 2.68-2.53 (m, 1H), 2.45-2.29 (m, 1H), 1.93-1.73 (m, 3H), 1.71-1.44 (m, 5H), 1.18-1.08 (m, 1H), 1.05 (s, 3H), 0.89 (s, 3H), 0.86 (d, J=6.8 Hz, 3H), 0.81 (t, J=7.2 Hz, 3H).

MS obsd. (ESI⁺) [(M+H)⁺]: 582.3.

### Example 196

### (1S)—N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

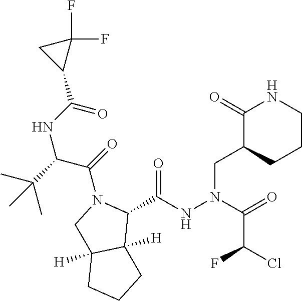 [SMILES: CC(C)(C)[C@H](NC(=O)[C@@H]1CC1(F)F)C(=O)N1C[C@@H]2CCC[C@@H]2[C@H]1C(=O)NN(C[C@@H]1CCCNC1=O)C(=O)[C@H](F)Cl]

The title compound was prepared in analogy to the procedure described for the preparation of Example 29, by using tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9), and (1S)-2,2-difluorocyclopropanecarboxylic acid and T₃P instead of acetic anhydride to afford (1S)—N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (Example 196) as a white solid.

¹H NMR (400 MHz, DMSO-d6) δ: ppm 11.07 (s, 1H), 8.48 (d, J=8.4 Hz, 1H), 7.56 (s, 1H), 7.00 (d, J=50.4 Hz, 1H), 4.49 (d, J=8.8 Hz, 1H), 4.01-3.93 (m, 2H), 3.89 (dd, J=10.4, 7.2 Hz, 1H), 3.67 (dd, J=10.4, 2.8 Hz, 1H), 3.38-3.33 (m, 1H), 3.14-3.07 (m, 2H), 2.95-32.84 (m, 1H), 2.78-2.69 (m, 1H), 2.56-2.51 (m, 1H), 2.44-2.36 (m, 1H), 1.90-1.71 (m, 8H), 1.68-1.54 (m, 2H), 1.50-1.41 (m, 1H), 1.39-1.31 (m, 1H), 0.95 (s, 9H).

MS obsd. (ESI⁺) [(M+H)⁺]: 592.4.

### Example 197

### (1S)—N-[(1S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide

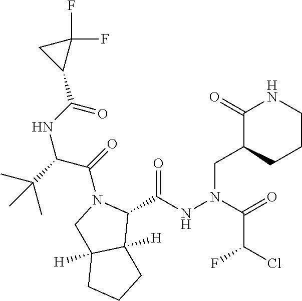 [SMILES: CC(C)(C)[C@H](NC(=O)[C@@H]1CC1(F)F)C(=O)N1C[C@@H]2CCC[C@@H]2[C@H]1C(=O)NN(C[C@@H]1CCCNC1=O)C(=O)[C@@H](F)Cl]

The title compound was prepared in analogy to the procedure described for the preparation of Example 29, by using tert-butyl N-amino-N-[[(3S)-2-oxo-3-piperidyl]methyl]carbamate (Intermediate 39a) instead of tert-butyl N-amino-N-[[(3S)-2-oxopyrrolidin-3-yl]methyl]carbamate (Intermediate 9), (2S)-2-chloro-2-fluoro-acetic acid instead of (2R)-2-chloro-2-fluoro-acetic acid, and (1S)-2,2-difluorocyclopropanecarboxylic acid and T₃P instead of acetic anhydride to afford (1S)—N-[(1S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide (Example 197) as a white solid.

¹H NMR (400 MHz, DMSO-d6) δ: ppm 10.86 (d, J=130.4 Hz, 1H), 8.50-8.43 (m, 1H), 7.50 (d, J=36.4 Hz, 1H), 6.79 (dd, J=278.8, 48.8 Hz, 1H), 4.48 (d, J=8.4 Hz, 1H), 3.99-3.94 (m, 1H), 3.89-3.63 (m, 3H), 3.15-3.03 (m, 2H), 2.94-2.80 (m, 1H), 2.78-2.52 (m, 3H), 2.42-1.98 (m, 1H), 1.95-1.63 (m, 8H), 1.63-1.37 (m, 3H), 1.37-1.27 (m, 1H), 0.96 (s, 9H).

MS obsd. (ESI⁺) [(M+H)⁺]: 592.4.

### BIOLOGICAL EXAMPLE

### Example 198

### SARS-CoV-2 3CL^{pro} Inhibition Assay

The full-length gene encoding SARS-CoV-2 3CL^{pro} was optimized and synthesized for Escherichia coli (E. coli) expression. The method of cloning and producing authentic SARS-CoV-2 3CL^{pro} was followed by the protocol published for SARS-CoV 3CL^{pro} previously (Grum-Tokars V. et al. “Evaluating the 3C-like protease activity of SARS-Coronavirus: Recommendations for standardized assays for drug discovery.” Virus Res. 2008 April; 133(1):63-73). The protein sequence as follows

SGFRKMAFPSGKVEGCMVQVTCGTTTLNGLWLDDVVYCPRHVICT
SEDMLNPNYEDLLIRKSNHNFLVQAGNVQLRVIGHSMQNCVLKLK
VDTANPKTPKYKFVRIQPGQTFSVLACYNGSPSGVYQCAMRPNFT
IKGSFLNGSCGSVGFNIDYDCVSFCYMHHMELPTGVHAGTDLEGN
FYGPFVDRQTAQAAGTDTTITVNVLAWLYAAVINGDRWFLNRFTT
TLNDFNLVAMKYNYEPLTQDHVDILGPLSAQTGIAVLDMCASLKE
LLQNGMNGRTILGSALLEDEFTPFDVVRQCSGVTFQ

For the SARS-CoV-2 3CL^{pro} assay, 2 μL of 0.02 μM recombinant SARS-CoV-2 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the SARS-CoV 3CL^{pro} was calculated by four parameters equation analysis.

### Example 199

### SARS-CoV 3CL^{pro} Inhibition Assay

The SARS-CoV 3CL^{pro} was expressed in E. coil
BL21 (DE3)with the protein sequence of
SGFRKMAFPSGKVEGCMVQVTCGTTTLNGLWLDDTVYCPRHVICT
AEDMLNPNYEDLLIRKSNHSFLVQAGNVQLRVIGHSMQNCLLRLK
VDTSNPKTPKYKFVRIQPGQTFSVLACYNGSPSGVYQCAMRPNHT
IKGSFLNGSCGSVGFNIDYDCVSFCYMHHMELPTGVHAGTDLEGK
FYGPFVDRQTAQAAGTDTTITLNVLAWLYAAVINGDRWFLNRFTT
TLNDFNLVAMKYNYEPLTQDHVDILGPLSAQTGIAVLDMCAALKE
LLQNGMNGRTILGSTILEDEFTPFDVVRQCSGVTFQ.

For the SARS-CoV 3CL^{pro} assay, 2 μL of 0.02 μM recombinant SARS-CoV 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA.

This FRET-based substrate contains a FAM fluorophore attached at the N-terminus of a canonical 3CL^{p}r° peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the SARS-CoV 3CL^{p}r° was calculated by four parameters equation analysis.

### Example 200

### MERS-CoV 3CL^{pro} Inhibition Assay

The MERS-CoV 3CL^{pro} was expressed in E. coli BL21 (DE3) with the protein sequence of

SGLVKMSHPSGDVEACMVQVTCGSMTLNGLWLDNTVWCPRHVMCP
ADQLSDPNYDALLISMTNHSFSVQKHIGAPANLRVVGHAMQGTLL
KLTVDVANPSTPAYTFTTVKPGAAFSVLACYNGRPTGTFTVVMRP
NYTIKGSFLCGSCGSVGYTKEGSVINFCYMHQMELANGTHTGSAF
DGTMYGAFMDKQVHQVQLTDKYCSVNVVAWLYAAILNGCAWFVKP
NRTSVVSFNEWALANQFTEFVGTQSVDMLAVKTGVAIEQLLYAIQ
QLYTGFQGKQILGSTMLEDEFTPEDVNMQIMGVVMQ

For the MERS-CoV 3CL^{pro} assay, 2 μL of 0.02 μM recombinant MERS-CoV 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was 25 nM and each compound was at a final concentration range of 100-0.0016 μM. The IC₅₀ value which is the value causing 50% inhibition of the catalytic activity of the MERS-CoV 3CL^{pro} was calculated by four parameters equation analysis.

### Example 201

### HCoV-229E 3CL^{pro} Inhibition Assay

The HCoV-229E 3CL^{pro} was expressed in E. coli BL21 (DE3) with the protein sequence of

AGLRKMAQPSGFVEKCVVRVCYGNTVLNGLWLGDIVYCPRHVIAS
NTTSAIDYDHEYSIMRLHNFSIISGTAFLGVVGATMHGVTLKIKV
SQTNMHTPRHSFRTLKSGEGFNILACYDGCAQGVFGVNMRTNWTI
RGSFINGACGSPGYNLKNGEVEFVYMHQIELGSGSHVGSSFDGVM
YGGFEDQPNLQVESANQMLTVNVVAFLYAAILNGCTWWLKGEKLF
VEHYNEWAQANGFTAMNGEDAFSILAAKTGVCVERLLHAIQVLNN
GFGGKQILGYSSLNDEFSINEVVKQMFGVNLQ

For the HCoV-229E 3CL^{pro} assay, 2 μL of 0.02 μM recombinant HCoV-229E 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{pro} peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was nM and each compound was at a final concentration range of 100-0.0016 μM. The IC₅₀ value which is the value causing 50% inhibition of the catalytic activity of the HCoV-229E 3CL^{pro} was calculated by four parameters equation analysis.

### Example 202

### HCoV—OC43 3CL^{pro} Inhibition Assay

The HCoV—OC43 3CL^{pro} was expressed in E. coli BL21 (DE3) with the protein sequence of

SGIVKMVNPTSKVEPCVVSVTYHNMTLNGLWLDDKVYCPRHVICS
ASDMTNPDYTNLLCVTSSDFTVLFDRLSLTVMSYQMRGCMLVLTV
TLQNSRTPKYTFGVVKPGETFTVLAAYNGKPQGAFHVTMRSSYTI
KGSFLCGSCGSVGYVIMGDCVKFVYMHQLELSTGCHTGTDFNGDF
YGPYKDAQVVQLPIQDYIQSVNFLAWLYAAILNNCNWFIQSDKCS
VEDFNVMALSNGFSQVKSDLVIDALASMTGVSLETLLAAIKRLKN
GFQGRQIMGSCSFEDELTPSDVYQQLAGIKLQ

For the HCoV—OC43 3CL^{pro} assay, 2 μL of 0.02 μM recombinant HCoV—OC43 3CL protease were mixed with serial dilutions of each compound in 4 μL assay buffer containing 40 mM HEPES, pH=8.0, 1 mM CHAPS, 150 mM NaCl, 1 mM EDTA, 1 mM TCEP in a well of a 384-well plate and pre-incubated at rt for 1 hr. The custom-synthesized fluorogenic 3CL^{pro} peptide substrate used in the assay is as follows: FAM-KTSAVLQSGFRKMEK-TAMRA. This FRET-based substrate contains a FAM fluorophore attached at the N-terminus of a canonical 3CL^{p}r° peptide substrate. The fluorophore is internally quenched by the TAMRA. The reaction was initiated by the addition of 10 μL of the substrate with a final concentration of 15 μM and each well was incubated at RT for 30 mins. The final concentration of the protease used at the assay was nM and each compound was at a final concentration range of 100-0.0016 μM. The IC₅₀ value which is the value causing 50% inhibition of the catalytic activity of the HCoV—OC43 3CL^{pro} was calculated by four parameters equation analysis.

TABLE 1
Activity of Examples and Compounds of present
invention in SARS-CoV-2 3CL^{pro} assay

| Example | IC₅₀ (μM) |
|---|---|
| 1 | 0.010 |
| 2 | 0.019 |
| 3 | 0.014 |
| 4 | 0.006 |
| 5 | 0.010 |
| 7 | 0.008 |
| 8 | 0.010 |
| 9 | 0.012 |
| 10 | 0.021 |
| 11 | 0.218 |
| 12 | 0.015 |
| 13 | 0.009 |
| 14 | 0.007 |
| 15 | 0.008 |
| 16 | 0.020 |
| 17 | 0.121 |
| 18 | 0.006 |
| 19 | 0.013 |
| 20 | 0.014 |
| 21 | 0.025 |
| 22 | 0.008 |
| 23 | 0.009 |
| 24 | 0.018 |
| 25 | 0.015 |
| 26 | 0.012 |
| 27 | 0.029 |
| 28 | 0.008 |
| 29 | 0.010 |
| 30 | 0.008 |
| 31 | 0.008 |
| 32 | 0.007 |
| 33 | 0.007 |
| 34 | 0.008 |
| 35 | 0.008 |
| 36 | 0.007 |
| 37 | 0.009 |
| 38 | 0.008 |
| 39 | 0.009 |
| 40 | 0.021 |
| 41 | 0.007 |
| 42 | 0.007 |
| 43 | 0.008 |
| 44 | 0.008 |
| 45 | 0.019 |
| 46 | 0.008 |
| 47 | 0.007 |
| 48 | 0.012 |
| 49 | 0.007 |
| 50 | 0.006 |
| 51 | 0.007 |
| 52 | 0.01 |
| 53 | 0.008 |
| 54 | 0.009 |
| 55 | 0.008 |
| 56 | 0.008 |
| 57 | 0.011 |
| 58 | 0.011 |
| 59 | 0.007 |
| 60 | 0.010 |
| 61 | 0.008 |
| 62 | 0.008 |
| 63 | 0.009 |
| 64 | 0.010 |
| 65 | 0.017 |
| 66 | 0.010 |
| 67 | 0.010 |
| 68 | 0.018 |
| 69 | 0.011 |
| 70 | 0.007 |
| 71 | 0.007 |
| 72 | 0.008 |
| 73 | 0.033 |
| 74 | 0.007 |
| 75 | 0.012 |
| 76 | 0.008 |
| 77 | 0.007 |
| 78 | 0.009 |
| 79 | 0.0071 |
| 80 | 0.010 |
| 81 | 0.009 |
| 82 | 0.008 |
| 83 | 0.009 |
| 84 | 0.007 |
| 85 | 0.012 |
| 86 | 0.007 |
| 87 | 0.014 |
| 88a | 0.042 |
| 88b | >10 |
| 89a | 0.051 |
| 89b | >10 |
| 90 | 0.010 |
| 91 | 0.012 |
| 92 | 0.008 |
| 93 | 0.031 |
| 94 | 0.021 |
| 95 | 0.010 |
| 96 | 0.011 |
| 97 | 0.011 |
| 98 | 0.009 |
| 99 | 0.008 |
| 100 | 0.008 |
| 101 | 0.009 |
| 102 | 0.012 |
| 103 | 0.013 |
| 104 | 0.012 |
| 105 | 0.008 |
| 106 | 0.033 |
| 107 | 0.018 |
| 108 | 0.043 |
| 109 | 0.014 |
| 110 | 0.024 |
| 111 | 0.016 |
| 112 | 0.008 |
| 113 | 0.010 |
| 114 | 0.017 |
| 115 | 0.009 |
| 116 | 0.014 |
| 117 | 0.011 |
| 118 | 0.013 |
| 119 | 0.015 |
| 120 | 0.019 |
| 121 | 0.026 |
| 122 | 0.026 |
| 123 | 0.037 |
| 124 | 0.026 |
| 125 | 0.010 |
| 126 | 0.010 |
| 127 | 0.010 |
| 128 | 0.011 |
| 129 | 0.011 |
| 130 | 0.012 |
| 131 | 0.010 |
| 132 | 0.010 |
| 133 | 0.020 |
| 134 | 0.010 |
| 135 | 0.011 |
| 136 | 0.016 |
| 137 | 0.018 |
| 138 | 0.016 |
| 139 | 0.014 |
| 140 | 0.011 |
| 141 | 0.010 |
| 142 | 0.010 |
| 143 | 0.008 |
| 144 | 0.011 |
| 145 | 0.018 |
| 146 | 0.016 |
| 147 | 0.012 |
| 148 | 0.013 |
| 149 | 0.006 |
| 150 | 0.011 |
| 151 | 0.013 |
| 152 | 0.015 |
| 153 | 0.012 |
| 154 | 0.017 |
| 155a | 0.024 |
| 155b | 1.569 |
| 156 | 0.014 |
| 157 | 0.044 |
| 158 | 0.011 |
| 159 | 0.013 |
| 160 | 0.013 |
| 161 | 0.016 |
| 162 | 0.012 |
| 163a | 0.009 |
| 163b | 0.017 |
| 164a | 0.008 |
| 164b | NA |
| 165a | 0.011 |
| 165b | 0.020 |
| 166a | 0.013 |
| 166b | 0.019 |
| 167a | 0.010 |
| 167b | 0.019 |
| 168a | 0.011 |
| 168b | 0.100 |
| 169a | 0.009 |
| 169b | NA |
| 170a | 0.016 |
| 170b | NA |
| 171a | 0.008 |
| 171b | NA |
| 172 | 0.017 |
| 173 | 0.013 |
| 174 | 0.010 |
| 175 | 0.010 |
| 176 | 0.011 |
| 177 | 0.147 |
| 178 | 0.148 |
| 179 | 0.201 |
| 180 | 0.491 |
| 181 | 0.208 |
| 182 | 0.011 |
| 183 | 0.011 |
| 184 | 0.016 |
| 185 | 0.011 |
| 186 | 0.013 |
| 187 | 0.024 |
| 188 | 0.008 |
| 189 | 0.008 |
| 190 | 0.008 |
| 191 | 0.007 |
| 192 | 0.010 |
| 193 | 0.017 |
| 194 | 0.015 |
| 195 | 0.028 |
| 196 | 0.011 |
| 197 | 0.011 |

NA: not available

TABLE 2
Activity of Examples and Compounds of present
invention in different coronaviruses

| Example | 229E | MERS | OC43 | SARS |
|---|---|---|---|---|
| 1 | 0.035 | 0.115 | 0.023 | 0.015 |
| 2 | 0.041 | 0.177 | 0.023 | 0.030 |
| 3 | 0.038 | 0.174 | 0.020 | 0.020 |
| 4 | 0.039 | 0.07 | 0.022 | 0.016 |
| 5 | 0.104 | 0.303 | 0.031 | 0.019 |
| 6 | 0.042 | 0.109 | 0.027 | 0.014 |
| 7 | 0.193 | 0.379 | 0.017 | 0.017 |
| 8 | 0.042 | 0.136 | 0.028 | 0.025 |
| 9 | 0.939 | 2.785 | 0.034 | 0.038 |
| 10 | 1.236 | 3.040 | 0.104 | 0.373 |
| 11 | 0.056 | 0.260 | 0.036 | 0.034 |
| 12 | 0.026 | 0.106 | 0.02 | 0.017 |
| 13 | 0.024 | 0.094 | 0.025 | 0.014 |
| 14 | 0.036 | 0.121 | 0.020 | 0.018 |
| 15 | 0.055 | 0.204 | 0.038 | 0.026 |
| 16 | 0.159 | 0.43 | 0.085 | 0.357 |
| 17 | 0.074 | 0.408 | 0.027 | 0.429 |
| 18 | 0.074 | 0.408 | 0.027 | 0.429 |
| 19 | 0.027 | 0.092 | 0.02 | 0.015 |
| 20 | 0.047 | 0.106 | 0.017 | 0.016 |
| 21 | 0.034 | 0.109 | 0.022 | 0.017 |
| 22 | 0.076 | 0.183 | 0.024 | 0.031 |
| 23 | 0.021 | 0.065 | 0.016 | 0.011 |
| 24 | 0.031 | 0.114 | 0.019 | 0.013 |
| 25 | 0.127 | 0.408 | 0.019 | 0.027 |
| 26 | 0.028 | 0.098 | 0.018 | 0.021 |
| 27 | 0.029 | 0.136 | 0.020 | 0.020 |
| 28 | 0.396 | 1.019 | 0.025 | 0.038 |
| 29 | 0.028 | 0.068 | 0.017 | 0.011 |
| 30 | 0.049 | 0.143 | 0.020 | 0.015 |
| 31 | 0.028 | 0.076 | 0.018 | 0.012 |
| 32 | 0.023 | 0.080 | 0.019 | 0.010 |
| 33 | 0.027 | 0.079 | 0.018 | 0.008 |
| 34 | 0.023 | 0.066 | 0.018 | 0.012 |
| 35 | 0.023 | 0.060 | 0.019 | 0.008 |
| 36 | 0.034 | 0.093 | 0.017 | 0.012 |
| 37 | 0.029 | 0.076 | 0.015 | 0.011 |
| 38 | 0.029 | 0.072 | 0.015 | 0.010 |
| 39 | 0.029 | 0.071 | 0.015 | 0.011 |
| 40 | 0.027 | 0.050 | 0.022 | 0.013 |
| 41 | 0.068 | 0.221 | 0.051 | 0.033 |
| 42 | 0.024 | 0.082 | 0.018 | 0.009 |
| 43 | 0.022 | 0.058 | 0.017 | 0.011 |
| 44 | 0.023 | 0.055 | 0.018 | 0.011 |
| 45 | 0.035 | 0.104 | 0.019 | 0.010 |
| 46 | 0.049 | 0.152 | 0.045 | 0.029 |
| 47 | 0.026 | 0.076 | 0.018 | 0.012 |
| 48 | 0.022 | 0.056 | 0.018 | 0.012 |
| 49 | 0.033 | 0.096 | 0.027 | 0.013 |
| 50 | 0.017 | 0.042 | 0.016 | 0.008 |
| 51 | 0.021 | 0.059 | 0.017 | 0.010 |
| 52 | 0.022 | 0.057 | 0.017 | 0.015 |
| 53 | 0.032 | 0.087 | 0.019 | 0.013 |
| 54 | 0.024 | 0.056 | 0.016 | 0.012 |
| 55 | 0.021 | 0.056 | 0.017 | 0.014 |
| 56 | 0.018 | 0.052 | 0.016 | 0.011 |
| 57 | 0.02 | 0.062 | 0.016 | 0.013 |
| 58 | 0.026 | 0.073 | 0.016 | 0.015 |
| 59 | 0.021 | 0.062 | 0.017 | 0.017 |
| 60 | 0.019 | 0.047 | 0.017 | 0.009 |
| 61 | 0.030 | 0.121 | 0.021 | 0.011 |
| 62 | 0.017 | 0.043 | 0.016 | 0.010 |
| 63 | 0.018 | 0.06 | 0.018 | 0.011 |
| 64 | 0.020 | 0.071 | 0.016 | 0.012 |
| 65 | 0.025 | 0.086 | 0.016 | 0.012 |
| 66 | 0.035 | 0.141 | 0.027 | 0.022 |
| 67 | 0.028 | 0.084 | 0.020 | 0.014 |
| 68 | 0.023 | 0.081 | 0.017 | 0.014 |
| 69 | 0.053 | 0.249 | 0.023 | 0.028 |
| 70 | 0.030 | 0.101 | 0.019 | 0.016 |
| 71 | 0.019 | 0.041 | 0.017 | 0.010 |
| 72 | 0.023 | 0.058 | 0.015 | 0.011 |
| 73 | 0.040 | 0.110 | 0.019 | 0.012 |
| 74 | 0.117 | 0.358 | 0.073 | 0.045 |
| 75 | 0.033 | 0.083 | 0.017 | 0.012 |
| 76 | 0.088 | 0.194 | 0.020 | 0.015 |
| 77 | 0.028 | 0.069 | 0.017 | 0.012 |
| 78 | 0.023 | 0.057 | 0.017 | 0.01 |
| 79 | 0.039 | 0.114 | 0.017 | 0.011 |
| 80 | 0.02 | 0.058 | 0.014 | 0.011 |
| 81 | 0.045 | 0.093 | 0.018 | 0.015 |
| 82 | 0.033 | 0.084 | 0.017 | 0.012 |
| 83 | 0.031 | 0.097 | 0.013 | 0.010 |
| 84 | 0.03 | 0.087 | 0.018 | 0.014 |
| 85 | 0.021 | 0.069 | 0.015 | 0.012 |
| 86 | 0.035 | 0.086 | 0.017 | 0.019 |
| 87 | 0.019 | 0.073 | 0.015 | 0.0093 |
| 88a | 0.105 | 0.249 | 0.017 | 0.028 |
| 88b | 0.053 | 0.155 | 0.015 | 0.076 |
| 89a | >10 | >10 | 3.429 | >10 |
| 89b | 0.047 | 0.193 | 0.017 | 0.101 |
| 90 | 0.022 | 0.055 | 0.016 | 0.012 |
| 91 | 0.039 | 0.115 | 0.018 | 0.015 |
| 92 | 0.023 | 0.057 | 0.02 | 0.018 |
| 93 | 0.042 | 0.094 | 0.019 | 0.054 |
| 94 | 0.059 | 0.136 | 0.021 | 0.029 |
| 95 | 0.028 | 0.071 | 0.019 | 0.014 |
| 96 | 0.025 | 0.063 | 0.01 | 0.014 |
| 97 | 0.074 | 0.208 | 0.018 | 0.013 |
| 98 | 0.022 | 0.059 | 0.015 | 0.011 |
| 99 | 0.030 | 0.081 | 0.021 | 0.012 |
| 100 | 0.032 | 0.077 | 0.021 | 0.014 |
| 101 | 0.040 | 0.094 | 0.018 | 0.013 |
| 102 | 0.112 | 0.207 | 0.018 | 0.016 |
| 103 | 0.167 | 0.372 | 0.019 | 0.025 |
| 104 | 0.055 | 0.128 | 0.018 | 0.020 |
| 105 | 0.027 | 0.071 | 0.016 | 0.011 |
| 106 | 0.066 | 0.179 | 0.030 | 0.057 |
| 107 | 0.040 | 0.111 | 0.020 | 0.025 |
| 108 | 0.050 | 0.127 | 0.024 | 0.074 |
| 109 | 0.029 | 0.086 | 0.018 | 0.020 |
| 110 | 0.037 | 0.096 | 0.021 | 0.033 |
| 111 | 0.037 | 0.096 | 0.022 | 0.028 |
| 112 | 0.022 | 0.071 | 0.017 | 0.014 |
| 113 | 0.023 | 0.079 | 0.020 | 0.015 |
| 114 | 0.029 | 0.113 | 0.022 | 0.025 |
| 115 | 0.030 | 0.102 | 0.019 | 0.015 |
| 116 | 0.036 | 0.098 | 0.022 | 0.018 |
| 117 | 0.025 | 0.086 | 0.014 | 0.011 |
| 118 | 0.027 | 0.109 | 0.021 | 0.018 |
| 119 | 0.056 | 0.206 | 0.023 | 0.020 |
| 120 | 0.038 | 0.098 | 0.017 | 0.034 |
| 121 | 0.043 | 0.131 | 0.020 | 0.050 |
| 122 | 0.046 | 0.146 | 0.019 | 0.036 |
| 123 | 0.051 | 0.146 | 0.027 | 0.062 |
| 124 | 0.041 | 0.115 | 0.021 | 0.045 |
| 125 | 0.042 | 0.114 | 0.018 | 0.012 |
| 126 | 0.028 | 0.068 | 0.016 | 0.011 |
| 127 | 0.035 | 0.068 | 0.018 | 0.012 |
| 128 | 0.036 | 0.097 | 0.019 | 0.018 |
| 129 | 0.029 | 0.104 | 0.020 | 0.016 |
| 130 | 0.032 | 0.087 | 0.020 | 0.019 |
| 131 | 0.026 | 0.158 | 0.020 | 0.014 |
| 132 | 0.025 | 0.106 | 0.023 | 0.014 |
| 133 | 0.040 | 0.106 | 0.024 | 0.032 |
| 134 | 0.036 | 0.097 | 0.019 | 0.014 |
| 135 | 0.039 | 0.131 | 0.021 | 0.015 |
| 136 | 0.055 | 0.074 | 0.035 | 0.015 |
| 137 | 0.046 | 0.192 | 0.025 | 0.027 |
| 138 | 0.054 | 0.283 | 0.023 | 0.025 |
| 139 | 0.035 | 0.139 | 0.020 | 0.018 |
| 140 | 0.031 | 0.094 | 0.015 | 0.025 |
| 141 | 0.035 | 0.117 | 0.020 | 0.015 |
| 142 | 0.031 | 0.083 | 0.020 | 0.013 |
| 143 | 0.028 | 0.086 | 0.020 | 0.013 |
| 144 | 0.033 | 0.112 | 0.016 | 0.015 |
| 145 | 0.077 | 0.152 | 0.017 | 0.027 |
| 146 | 0.069 | 0.151 | 0.015 | 0.023 |
| 147 | 0.040 | 0.089 | 0.014 | 0.016 |
| 148 | 0.03 | 0.13 | 0.021 | 0.02 |
| 149 | 0.045 | 0.139 | 0.023 | 0.015 |
| 150 | 0.033 | 0.063 | 0.025 | 0.016 |
| 151 | 0.031 | 0.067 | 0.024 | 0.014 |
| 152 | 0.028 | 0.099 | 0.021 | 0.02 |
| 153 | 0.043 | 0.25 | 0.02 | 0.015 |
| 154 | 0.052 | 0.305 | 0.033 | 0.021 |
| 155a | 0.739 | 1.328 | 0.037 | 0.028 |
| 155b | >10.000 | >10.000 | 1.828 | 2.442 |
| 156 | 0.032 | 0.106 | 0.024 | 0.028 |
| 157 | 0.060 | 0.149 | 0.022 | 0.058 |
| 158 | 0.034 | 0.08 | 0.025 | 0.017 |
| 159 | 0.040 | 0.103 | 0.019 | 0.013 |
| 160 | 0.024 | 0.105 | 0.023 | 0.019 |
| 161 | 0.099 | 0.336 | 0.024 | 0.027 |
| 162 | 0.033 | 0.094 | 0.019 | 0.015 |
| 163a | 0.052 | 0.226 | 0.028 | 0.018 |
| 163b | 0.104 | 0.469 | 0.042 | 0.033 |
| 164a | 0.098 | 0.243 | 0.030 | 0.016 |
| 164b | NA | NA | NA | NA |
| 165a | 0.038 | 0.185 | 0.026 | 0.012 |
| 165b | 0.057 | 0.291 | 0.037 | 0.036 |
| 166a | 0.047 | 0.229 | 0.027 | 0.018 |
| 166b | 0.051 | 0.239 | 0.040 | 0.030 |
| 167a | 0.053 | 0.169 | 0.018 | 0.015 |
| 167b | 0.210 | 1.630 | 0.070 | 0.041 |
| 168a | 0.104 | 0.290 | 0.031 | 0.016 |
| 168b | 0.219 | 2.109 | 0.149 | 0.185 |
| 169a | 0.056 | 0.118 | 0.027 | 0.020 |
| 169b | NA | NA | NA | NA |
| 170a | 0.107 | 0.276 | 0.035 | 0.025 |
| 170b | NA | NA | NA | NA |
| 171a | 0.094 | 0.130 | 0.027 | 0.017 |
| 171b | NA | NA | NA | NA |
| 172 | 0.071 | 0.187 | 0.031 | 0.026 |
| 173 | 0.027 | 0.175 | 0.027 | 0.018 |
| 174 | 0.029 | 0.123 | 0.023 | 0.012 |
| 175 | 0.037 | 0.105 | 0.023 | 0.015 |
| 176 | 0.028 | 0.096 | 0.020 | 0.016 |
| 177 | 0.138 | 0.278 | 0.041 | 0.232 |
| 178 | 0.065 | 0.264 | 0.024 | 0.259 |
| 179 | 0.093 | 0.315 | 0.025 | 0.222 |
| 180 | 0.241 | 0.657 | 0.035 | 0.786 |
| 181 | 0.048 | 0.363 | 0.032 | 0.252 |
| 182 | 0.037 | 0.089 | 0.014 | 0.012 |
| 183 | 0.059 | 0.174 | 0.015 | 0.016 |
| 184 | 0.086 | 0.275 | 0.017 | 0.041 |
| 185 | 0.040 | 0.099 | 0.017 | 0.021 |
| 186 | 0.049 | 0.069 | 0.019 | 0.023 |
| 187 | 0.036 | 0.167 | 0.020 | 0.043 |
| 188 | 0.026 | 0.068 | 0.016 | 0.012 |
| 189 | 0.024 | 0.05 | 0.015 | 0.010 |
| 190 | 0.029 | 0.059 | 0.017 | 0.011 |
| 191 | 0.027 | 0.049 | 0.018 | 0.011 |
| 192 | 0.052 | 0.148 | 0.019 | 0.017 |
| 193 | 0.049 | 0.11 | 0.015 | 0.033 |
| 194 | 0.029 | 0.112 | 0.026 | 0.025 |
| 195 | 0.029 | 0.112 | 0.023 | 0.051 |
| 196 | 0.038 | 0.089 | 0.02 | 0.014 |
| 197 | 0.065 | 0.094 | 0.016 | 0.013 |

NA: not available

### Example 203

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 204

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1-31. (canceled)

32. A compound of formula (I) 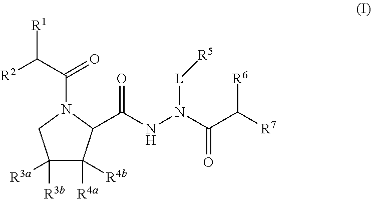
or a pharmaceutically acceptable salt thereof, wherein:
L is C₁-C₆-alkyl;
L¹ is selected from a covalent bond, O, NH, and 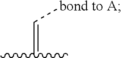
A is selected from C₆-C₁₄-aryl and 3- to 14-membered heteroaryl;
R¹ is selected from a group 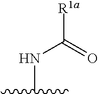
and a group 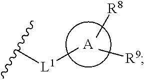
and
R² is selected from hydrogen, C₃-C₁₀-cycloalkyl, C₃-C₁₀-cycloalkyl-C₁-C₆-alkyl, C₁-C₆-alkyl-C₃-C₁₀-cycloalkyl, C₁-C₆-alkyl, C₁-C₆-alkyl-S—C₁-C₆-alkyl, 3- to 14-membered heteroaryl, (3- to 14-membered heteroaryl)-C₁-C₆-alkyl, C₆-C₁₄-aryl, and C₆-C₁₄-aryl-C₁-C₆-alkyl, wherein said C₃-C₁₀-cycloalkyl is optionally substituted with 1 C₁-C₆-alkyl substituent; or
R¹ and R², taken together with the carbon atom to which they are attached, form a 3- to 14-membered heteroaryl or a C₆-C₁₄-aryl, wherein said 3- to 14-membered heteroaryl or C₆-C₁₄-aryl is optionally substituted with 1 to 2 substituents selected from halogen and halo-C₁-C₆-alkyl;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl and halo-C₁-C₆-alkyl;
R^{3a}, R^{3b}, R^{4a}, and R^{4b} are each independently selected from hydrogen, halogen, C₁-C₆-alkyl, and halo-C₁-C₆-alkyl; or
R^{3a} and R^{3b}, taken together with the carbon atom to which they are attached, form a C₃-C₁₀-cycloalkyl; and R^{4a} and R^{4b} are each independently selected from hydrogen and C₁-C₆-alkyl; wherein said C₃-C₁₀-cycloalkyl is optionally substituted with 1 to 2 C₁-C₆-alkyl substituents; or
R^{4a} and R^{4b}, taken together with the carbon atom to which they are attached, form a C₃-C₁₀-cycloalkyl; and R^{3a} and R^{3b} are each independently selected from hydrogen and C₁-C₆-alkyl; wherein said C₃-C₁₀-cycloalkyl is optionally substituted with 1 to 2 C₁-C₆-alkyl substituents; or
R^{3a} and R^{4a}, taken together with the carbon atoms to which they are attached, form a C₃-C₁₀-cycloalkyl; and R^{3b} and R^{4b} are each independently selected from hydrogen and C₁-C₆-alkyl; wherein said C₃-C₁₀-cycloalkyl is optionally substituted with 1 to 2 C₁-C₆-alkyl substituents;
R⁵ is selected from 3- to 14-membered heterocycloalkyl, 3- to 14-membered heteroaryl, carbamoyl, and C₁-C₆-alkyl-NH—C(O)—; wherein said 3- to 14-membered heterocycloalkyl is optionally substituted with 1 oxo substituent;
R⁶ is selected from fluoro and chloro;
R⁷ is selected from hydrogen, chloro, and acyl; and
R⁸ and R⁹ are each independently selected from hydrogen, halogen and halo-C₁-C₆-alkyl.

33. The compound of formula (I) according to claim 32, or a pharmaceutically acceptable salt thereof, wherein:
L¹ is selected from a covalent bond, O, NH, and 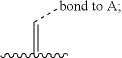
A is selected from C₆-C₁₄-aryl and 3- to 14-membered heteroaryl;
R¹ is selected from a group 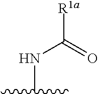
and a group 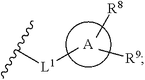
and
R² is selected from hydrogen, C₃-C₁₀-cycloalkyl, C₃-C₁₀-cycloalkyl-C₁-C₆-alkyl, (3- to 14-membered heteroaryl)-C₁-C₆-alkyl, C₁-C₆-alkyl, C₆-C₁₄-aryl, C₆-C₁₄-aryl-C₁-C₆-alkyl, and C₁-C₆-alkyl-S—C₁-C₆-alkyl, wherein said C₃-C₁₀-cycloalkyl is optionally substituted with 1 C₁-C₆-alkyl substituent; or
R¹ and R², taken together with the carbon atom to which they are attached, form a C₆-C₁₄-aryl which is substituted with 2 substituents selected from halogen and halo-C₁-C₆-alkyl;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl and halo-C₁-C₆-alkyl; and
R⁸ and R⁹ are each independently selected from hydrogen, halogen and halo-C₁-C₆-alkyl.

34. The compound of formula (I) according to claim 32, or a pharmaceutically acceptable salt thereof, wherein:
L¹ is selected from a covalent bond, O, NH, and 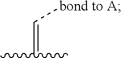
A is selected from phenyl, pyridyl, pyrimidinyl;
R¹ is selected from a group 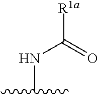
and a group 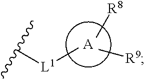
and
R² is selected from hydrogen, cyclopropyl, 1-methylcyclopropyl, cyclobutyl, 1-bicyclo[1.1.1]pentanylmethyl, methyl, ethyl, propyl, sec-butyl, tert-butyl, isobutyl, 1-ethylpropyl, 1,1-dimethylpropyl, 2,2-dimethylpropyl, 2-methylsulfanylethyl, phenyl, benzyl, and pyridylmethyl; or
R¹ and R², taken together with the carbon atom to which they are attached, form a phenyl, which is substituted with 2 substituents selected from fluoro and CF₃;
R^{1a} is selected from bicyclo[1.1.1]pentanyl, cyclopropyl, 1-fluorocyclopropyl, 2,2-difluorocyclopropyl, 1-methylclopropyl, 1-fluorocyclobutyl, 3,3-difluorocyclobutyl, cyclobutyl, phenyl, isoxazolyl, methyl, ethyl, propyl, isopropyl, isobutyl, sec-butyl, tert-butyl, CHFCl, CHCl₂, CHF₂, CF₃, 1-fluoro-1-methyl-ethyl, 2,2-difluoroethyl, 2,2,2-trifluoroethyl, 1-fluoroethyl, and 1,1,2,2,2-pentachloroethyl; and
R⁸ and R⁹ are each independently selected from hydrogen, fluoro and CF₃.

35. The compound of formula (I) according to claim 33, or a pharmaceutically acceptable salt thereof, wherein:
R¹ is a group 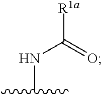
R² is selected from C₃-C₁₀-cycloalkyl and C₁-C₆-alkyl; and
R^{1a} is selected from C₃-C₁₀-cycloalkyl, C₁-C₆-alkyl and halo-C₁-C₆-alkyl, wherein said C₃-C₁₀-cycloalkyl is optionally substituted with 1 to 2 halogen substituents.

36. The compound of formula (I) according to claim 35, or a pharmaceutically acceptable salt thereof, wherein:
R¹ is a group 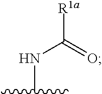
R² is selected from cyclopropyl, 1,1-dimethylpropyl, 1-ethylpropyl, sec-butyl and tert-butyl; and
R^{1a} is selected from cyclopropyl, 1-fluorocyclopropyl, 2,2-difluorocyclopropyl, isopropyl, tert-butyl, 1-fluoroethyl, CHFCl, CF₃, and CHF₂.

37. The compound of formula (I) according to claim 32, or a pharmaceutically acceptable salt thereof, wherein
R^{3a} is C₁-C₆-alkyl; R^{3b} is hydrogen or C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 1 to 2 C₁-C₆-alkyl substituents.

38. The compound of formula (I) according to claim 37, or a pharmaceutically acceptable salt thereof, wherein
R^{3a} and R^{3b} are both C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 2 C₁-C₆-alkyl substituents.

39. The compound of formula (I) according to claim 38, or a pharmaceutically acceptable salt thereof, wherein
R^{3a} and R^{3b} are both methyl; and R^{4a} and R^{4b} are both hydrogen; or
R^{3a} and R^{3b}, taken together with the carbon atom to which they are attached, form a cyclopropyl; and R^{4a} and R^{4b} are both hydrogen; or
R^{3a} and R^{4a}, taken together with the carbon atoms to which they are attached, form a cyclopropyl or a cyclopentyl; and R^{3b} and R^{4b} are both hydrogen; wherein said cyclopropyl is substituted with 2 methyl substituents.

40. The compound of formula (I) according to claim 32, or a pharmaceutically acceptable salt thereof, wherein:
L is C₁-C₆-alkyl; and
R⁵ is selected from 3- to 14-membered heterocycloalkyl and 3- to 14-membered heteroaryl; wherein said 3- to 14-membered heterocycloalkyl is substituted with 1 oxo substituent.

41. The compound of formula (I) according to claim 39, or a pharmaceutically acceptable salt thereof, wherein:
L is C₁-C₆-alkyl; and
R⁵ is 3- to 14-membered heterocycloalkyl substituted with 1 oxo substituent.

42. The compound of formula (I) according to claim 41, or a pharmaceutically acceptable salt thereof, wherein:
L is CH₂; and
R⁵ is

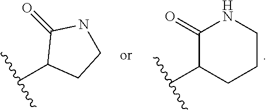 [SMILES: *C1CCNC1=O ; *C1CCCNC1=O]

43. The compound of formula (I) according to claim 32, or a pharmaceutically acceptable salt thereof, wherein:
R⁶ is selected from fluoro and chloro; and
R⁷ is selected from hydrogen and chloro.

44. The compound of formula (I) according to claim 43, or a pharmaceutically acceptable salt thereof, wherein:
R⁶ is fluoro; and
R⁷ is chloro.

45. The compound of formula (I) according to claim 32, or a pharmaceutically acceptable salt thereof, wherein:
L is C₁-C₆-alkyl;
L¹ is selected from a covalent bond, O, NH, and 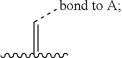
A is selected from C₆-C₁₄-aryl and 3- to 14-membered heteroaryl;
R¹ is selected from a group 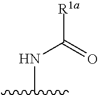
and a group 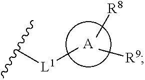
and
R² is selected from hydrogen, C₃-C₁₀-cycloalkyl, C₃-C₁₀-cycloalkyl-C₁-C₆-alkyl, (3- to 14-membered heteroaryl)-C₁-C₆-alkyl, C₁-C₆-alkyl, C₆-C₁₄-aryl, C₆-C₁₄-aryl-C₁-C₆-alkyl, and C₁-C₆-alkyl-S—C₁-C₆-alkyl, wherein said C₃-C₁₀-cycloalkyl is optionally substituted with 1 C₁-C₆-alkyl substituent; or
R¹ and R², taken together with the carbon atom to which they are attached, form a C₆-C₁₄-aryl which is substituted with 2 substituents selected from halogen and halo-C₁-C₆-alkyl;
R^{1a} is selected from C₃-C₁₀-cycloalkyl, C₆-C₁₄-aryl, 3- to 14-membered heteroaryl, C₁-C₆-alkyl, and halo-C₁-C₆-alkyl, wherein said C₃-C₁₀-cycloalkyl and 3- to 14-membered heteroaryl are optionally substituted with 1 to 2 substituents selected from halogen, C₁-C₆-alkyl halo-C₁-C₆-alkyl;
R² is selected from hydrogen, C₃-C₁₀-cycloalkyl, C₃-C₁₀-cycloalkyl-C₁-C₆-alkyl, (3- to 14-membered heteroaryl)-C₁-C₆-alkyl and C₁-C₆-alkyl, wherein said C₃-C₁₀-cycloalkyl is optionally substituted with 1 C₁-C₆-alkyl substituent;
R^{3a} is C₁-C₆-alkyl; R^{3b} is hydrogen or C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 1 to 2 C₁-C₆-alkyl substituents;
R⁵ is selected from 3- to 14-membered heterocycloalkyl and 3- to 14-membered heteroaryl; wherein said 3- to 14-membered heterocycloalkyl is substituted with 1 oxo substituent;
R⁶ is selected from fluoro and chloro;
R⁷ is selected from hydrogen and chloro; and
R⁸ and R⁹ are each independently selected from hydrogen, halogen and halo-C₁-C₆-alkyl.

46. The compound of formula (I) according to claim 45, or a pharmaceutically acceptable salt thereof, wherein:
L is C₁-C₆-alkyl;
R¹ is a group 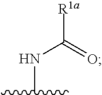
R^{1a} is selected from C₃-C₁₀-cycloalkyl, C₁-C₆-alkyl and halo-C₁-C₆-alkyl, wherein said C₃-C₁₀-cycloalkyl is optionally substituted with 1 to 2 halogen substituents;
R² is selected from C₃-C₁₀-cycloalkyl and C₁-C₆-alkyl;
R^{3a} and R^{3b} are both C₁-C₆-alkyl; and R^{4a} and R^{4b} are both hydrogen; or
R^{3a} and R^{3b}, taken together with the carbon atom to which they are attached, form a C₃-C₁₀-cycloalkyl; and R^{4a} and R^{4b} are both hydrogen; or
R^{3a} and R^{4a}, taken together with the carbon atoms to which they are attached, form a C₃-C₁₀-cycloalkyl; and R^{3b} and R^{4b} are both hydrogen; wherein said C₃-C₁₀-cycloalkyl is optionally substituted with 2 C₁-C₆-alkyl substituents;
R⁵ is 3- to 14-membered heterocycloalkyl substituted with 1 oxo substituent;
R⁶ is fluoro; and
R⁷ is chloro.

47. The compound of formula (I) according to claim 45, or a pharmaceutically acceptable salt thereof, wherein:
L is CH₂;
R¹ is a group 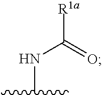
R^{1a} is selected from cyclopropyl, 1-fluorocyclopropyl, 2,2-difluorocyclopropyl, isopropyl, tert-butyl, 1-fluoroethyl, CHFCl, CF₃, and CHF₂;
R² is selected from cyclopropyl, 1,1-dimethylpropyl, 1-ethylpropyl, sec-butyl and tert-butyl;
R^{3a} and R^{3b} are both methyl; and R^{4a} and R^{4b} are both hydrogen; or
R^{3a} and R^{3b}, taken together with the carbon atom to which they are attached, form a cyclopropyl; and R^{4a} and R^{4b} are both hydrogen; or
R^{3a} and R^{4a}, taken together with the carbon atoms to which they are attached, form a cyclopropyl or a cyclopentyl; and R^{3b} and R^{4b} are both hydrogen; wherein said cyclopropyl is substituted with 2 methyl substituents;
R⁵ is 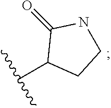 [SMILES: *C1CCNC1=O]
R⁶ is fluoro; and
R⁷ is chloro.

48. The compound of formula (I) according to claim 32, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methyl-butyl]-2,2,2-trifluoro-acetamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]bicyclo[1.1.1]pentane-1-carboxamide;
N-[(1S)-1-[(1R,2S,5S)-2-[[but-2-ynoyl-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
(1R,2S,5S)—N′-[(2R)-2-chloro-2-fluoro-acetyl]-3-[2-(3,5-difluorophenoxy)acetyl]-6,6-dimethyl-N′—[[(3S)-2-oxopyrrolidin-3-yl]methyl]-3-azabicyclo[3.1.0]hexane-2-carbohydrazide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1S,2S,5R)-2-[[[(2R)-2-chloro-2-fluoro-acetyl]-[[(3S)-2-oxopyrrolidin-3-yl]methyl]amino]carbamoyl]-3-azabicyclo[3.2.0]heptane-3-carbonyl]-2,2-dimethyl-propyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-(1H-pyrazol-3-ylmethyl)amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-5-(trifluoromethyl)isoxazole-3-carboxamide;
N-[(1S)-1-[(1R,2S,5S)-2-[[(2-chloroacetyl)-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-phenyl-ethyl]-2,2,2-trifluoro-acetamide;
N-[(1S)-1-benzyl-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
3-[[(2R)-2-chloro-2-fluoro-acetyl]-[[(1R,2S,5S)-3-[(2S)-3,3-dimethyl-2-[(2,2,2-trifluoroacetyl)amino]butanoyl]-6,6-dimethyl-3-azabicyclo[3.1.0]hexane-2-carbonyl]amino]amino]propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3-methylsulfanyl-propyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2-methyl-propanamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-(1-methylcyclopropyl)-2-oxo-ethyl]-2,2,2-trifluoro-acetamide;
N-[(1S)-2-[(6S)-6-[[[(2R)-2-chloro-2-fluoro-acetyl]-[[(3S)-2-oxopyrrolidin-3-yl]methyl]amino]carbamoyl]-5-azaspiro[2.4]heptan-5-yl]-1-(1-methylcyclopropyl)-2-oxo-ethyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-1-(4-pyridylmethyl)ethyl]-2,2,2-trifluoro-acetamide;
N-[(1S)-1-(1-bicyclo[1.1.1]pentanylmethyl)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-acetamide;
(3S,3aS,6aR)—N′-[(2R)-2-chloro-2-fluoro-acetyl]-2-[2-(3-fluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide;
(3R,3aR,6aS)—N′-[(2R)-2-chloro-2-fluoro-acetyl]-2-[2-(3,5-difluorophenoxy)acetyl]-N′—[[(3S)-2-oxopyrrolidin-3-yl]methyl]-3,3a,4,5,6,6a-hexahydro-1H-cyclopenta[c]pyrrole-3-carbohydrazide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide;
N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-propanamide;
N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide;
N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-cyclobutyl-2-oxo-ethyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide;
N-[(1S)-1-[(2S,4S)-2-[[[(2R)-2-chloro-2-fluoro-acetyl]-[[(3S)-2-oxopyrrolidin-3-yl]methyl]amino]carbamoyl]-4-methyl-pyrrolidine-1-carbonyl]-2,2-dimethyl-propyl]-2,2,2-trifluoro-acetamide;
N-[(1S)-2-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrol-2-yl]-1-methyl-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]propanamide;
Rac-2,2-difluoro-N-[(1S,2S)-2-methyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-methyl-cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-methyl-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
Rac-2,2-difluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-methyl-propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dimethyl-propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3,3-trifluoro-propanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]butanamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2-fluoro-2-methyl-propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2-fluoro-2-methyl-propanamide;
Rac-2-fluoro-N-[(1S)-2,2-dimethyl-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]propyl]propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-dichloro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-dichloro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2,3,3,3-pentafluoro-propanamide;
Rac-2-fluoro-N-[(1S,2S)-2-methyl-1-[rac-(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]propanamide;
N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,3,3,3-pentafluoro-propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,3,3,3-pentafluoro-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-propanamide;
Rac-2-fluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,3,3,3-pentafluoro-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
2,2-dichloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]acetamide;
Rac-2,2-difluoro-N-[(1S,2S)-2-methyl-1-[(1R,2S,5S)-6,6-dimethyl-2-[[[(2R)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-dimethyl-propanamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2,2-trifluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]benzamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclobutanecarboxamide;
(1S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-3,3-difluoro-cyclobutanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-1-fluoro-cyclopropanecarboxamide;
(1S)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1R)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclobutanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-3,3-difluoro-cyclobutanecarboxamide;
(1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1R)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
(1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-1-fluoro-cyclopropanecarboxamide;
(1R)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-methyl-propanamide;
2,2-difluoro-N-[rac-(1S,2S)-2-methyl-1-[rac-(1R,2S,5S)-6,6-dimethyl-2-[[[rac-(2S)-2-chloro-2-fluoro-acetyl]-[[rac-(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
2,2-difluoro-N-[rac-(1S,2R)-2-methyl-1-[rac-(1R,2S,5S)-6,6-dimethyl-2-[[[rac-(2R)-2-chloro-2-fluoro-acetyl]-[[rac-(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-3,3-difluoro-cyclobutanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2R)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
(2S)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]cyclopropanecarboxamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclobutanecarboxamide;
N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2S)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
(2R)—N-[(1S,2R)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
rac-2,2-difluoro-N-[(1S,2R)-2-methyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]cyclopropanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-1-fluoro-cyclobutanecarboxamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide;
N-[(1S,2S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2R)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-3,3-difluoro-cyclobutanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-3,3-difluoro-cyclobutanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2-difluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-3,3-difluoro-cyclobutanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
rac-2-fluoro-N-[(1S)-2-ethyl-1-[(2S)-4,4-dimethyl-2-[[[(2R)-2-chloro-2-fluoro-acetyl]-[[(3S)-2-oxopyrrolidin-3-yl]methyl]amino]carbamoyl]pyrrolidine-1-carbonyl]butyl]propanamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-1-fluoro-cyclopropanecarboxamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2-ethyl-butyl]-2,2-difluoro-acetamide;
(2S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide;
(2R)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide;
rac-2-fluoro-N-[(1S)-2,2-dimethyl-1-[(1R,2S,5S)-6,6-dimethyl-2-[[[(2S)-2-chloro-2-fluoro-acetyl]-[[(3S)-2-oxopyrrolidin-3-yl]methyl]amino]carbamoyl]-3-azabicyclo[3.1.0]hexane-3-carbonyl]butyl]propanamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-1-fluoro-cyclopropanecarboxamide;
(1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
(1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-butyl]-1-fluoro-cyclopropanecarboxamide;
N-[(1S)-1-[(2S)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide;
N-[(1S)-1-[(2R)-2-[[[(2R)-2-chloro-2-fluoro-acetyl]-[[(3S)-2-oxopyrrolidin-3-yl]methyl]amino]carbamoyl]-3,3-dimethyl-pyrrolidine-1-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide;
N-[(1S)-2-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclobutyl-2-oxo-ethyl]-1-fluoro-cyclopropanecarboxamide;
(1S)—N-[(1S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide;
(1S)—N-[(1S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2,2-dimethyl-propyl]-2,2-difluoro-cyclopropanecarboxamide;
N-[(1S,2S)-1-[(2S,4R)-2-[[[(2R)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide;
N-[(1S,2S)-1-[(2S,4R)-2-[[[(2S)-2-chloro-2-fluoro-acetyl]-[[(3S)-2-oxopyrrolidin-3-yl]methyl]amino]carbamoyl]-4-(trifluoromethyl)pyrrolidine-1-carbonyl]-2-methyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-3,3-dimethyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide;
N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]cyclopropanecarboxamide;
N-[(1S)-1-[(2S)-2-[[[(2R)-2-chloro-2-fluoro-acetyl]-[[(3S)-2-oxo-3-piperidyl]methyl]amino]carbamoyl]-4,4-dimethyl-pyrrolidine-1-carbonyl]-2,2-dimethyl-butyl]cyclopropanecarboxamide;
N-[(1S)-1-[(2S)-2-[[[(2R)-2-chloro-2-fluoro-acetyl]-[[(3R)-2-oxo-3-piperidyl]methyl]amino]carbamoyl]-4,4-dimethyl-pyrrolidine-1-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2,2-dimethyl-butyl]cyclopropanecarboxamide;
N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide;
N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide;
N-[(1S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-ethyl-butyl]cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]cyclopropanecarboxamide;
N-[(1S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2,2-difluoro-acetamide;
(1R,2S,5S)-3-[3,5-bis(trifluoromethyl)benzoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide;
(1R,2S,5S)-3-[2-[3,5-bis(trifluoromethyl)phenyl]acetyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide;
(1R,2S,5S)—N′-[(2R)-2-chloro-2-fluoro-acetyl]-3-[3-fluoro-5-(trifluoromethyl)benzoyl]-6,6-dimethyl-N′—[[(3S)-2-oxopyrrolidin-3-yl]methyl]-3-azabicyclo[3.1.0]hexane-2-carbohydrazide;
(1R,2S,5S)-3-[(E)-3-[3,5-bis(trifluoromethyl)phenyl]prop-2-enoyl]-N′-[(2R)-2-chloro-2-fluoro-acetyl]-6,6-dimethyl-N′—[[(3S)-2-oxopyrrolidin-3-yl]methyl]-3-azabicyclo[3.1.0]hexane-2-carbohydrazide;
(1R,2S,5S)—N′-[(2R)-2-chloro-2-fluoro-acetyl]-3-[(E)-3-(3,5-difluorophenyl)prop-2-enoyl]-6,6-dimethyl-N′—[[(3S)-2-oxopyrrolidin-3-yl]methyl]-3-azabicyclo[3.1.0]hexane-2-carbohydrazide;
(3S,3aS,6aR)—N′-[(2R)-2-chloro-2-fluoro-acetyl]-2-[(2S)-2-[(6-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide;
(3S,3aS,6aR)—N′-[(2R)-2-chloro-2-fluoro-acetyl]-2-[(2S)-2-[(5-fluoropyrimidin-4-yl)amino]-3,3-dimethyl-butanoyl]-N′—[[(3S)-2-oxopyrrolidin-3-yl]methyl]-3,3a,4,5,6,6a-hexahydro-1H-cyclopenta[c]pyrrole-3-carbohydrazide;
(3S,3aS,6aR)—N′-[(2R)-2-chloro-2-fluoro-acetyl]-2-[(2S)-3,3-dimethyl-2-(2-pyridylamino)butanoyl]-N′—[[(3S)-2-oxopyrrolidin-3-yl]methyl]-3,3a,4,5,6,6a-hexahydro-1H-cyclopenta[c]pyrrole-3-carbohydrazide;
(2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide;
(2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2R)—N-[(1S,2S)-1-[(3S,3aS,6aR)-3-[[[(2R)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2S)—N-[(1S,2S)-1-[(3S,3aS,6aR)-3-[[[(2S)-2-chloro-2-fluoro-acetyl]-[[(3R)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide;
(2R)-2-chloro-N-[(1S,2S)-1-[(1R,2S,5S)-2-[[[(2R)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3R)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(1S)—N-[(1S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide; and
(1S)—N-[(1S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2,2-dimethyl-propyl]-2,2-difluoro-cyclopropanecarboxamide.

49. The compound of formula (I) according to claim 48, or a pharmaceutically acceptable salt thereof, selected from:
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2,2-trifluoro-acetamide;
N-[(1S)-2-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexan-3-yl]-1-cyclopropyl-2-oxo-ethyl]-2,2,2-trifluoro-acetamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-propyl]-2,2,2-trifluoro-acetamide;
N-[(1S)-1-[(6S)-6-[[[(2R)-2-chloro-2-fluoro-acetyl]-[[(3S)-2-oxopyrrolidin-3-yl]methyl]amino]carbamoyl]-5-azaspiro[2.4]heptane-5-carbonyl]-2,2-dimethyl-propyl]-2,2,2-trifluoro-acetamide;
N-[(1S)-1-[(2S)-2-[[[(2R)-2-chloro-2-fluoro-acetyl]-[[(3S)-2-oxopyrrolidin-3-yl]methyl]amino]carbamoyl]-4,4-dimethyl-pyrrolidine-1-carbonyl]-2,2-dimethyl-propyl]-2,2,2-trifluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-dimethyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2-methyl-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-acetamide;
N-[(1S,2S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-methyl-butyl]-2,2-difluoro-acetamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2,2-trifluoro-acetamide;
(1S)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
(1R)—N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-propyl]-2,2-difluoro-cyclopropanecarboxamide;
N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2,2-difluoro-acetamide;
N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-1-fluoro-cyclopropanecarboxamide;
(2S)—N-[(1S,2R)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-2,2-difluoro-acetamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-ethyl-butyl]-1-fluoro-cyclopropanecarboxamide;
N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2-ethyl-butyl]-2,2-difluoro-acetamide;
(2S)—N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2-fluoro-propanamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-1-fluoro-cyclopropanecarboxamide;
N-[(1S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
N-[(1S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2,2-dimethyl-butyl]-2,2-difluoro-acetamide;
(1S)—N-[(1S)-1-[(3S,3aS,6aR)-3-[[[(2R)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
(1S)—N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-cyclopropanecarboxamide;
N-[(1S)-1-[(3S,3aS,6aR)-3-[[[(2S)-2-chloro-2-fluoro-acetyl]-[[(3S)-2-oxopyrrolidin-3-yl]methyl]amino]carbamoyl]-3,3a,4,5,6,6a-hexahydro-1H-cyclopenta[c]pyrrole-2-carbonyl]-2,2-dimethyl-butyl]-2,2-difluoro-acetamide;
(2R)-2-chloro-N-[(1S,2S)-1-[(1R,2S,5S)-2-[[[(2R)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxopyrrolidin-3-yl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(2S)-2-[[[(2S)-2-chloro-2-fluoro-acetyl]-[[(3S)-2-oxopyrrolidin-3-yl]methyl]amino]carbamoyl]-4,4-dimethyl-pyrrolidine-1-carbonyl]-2-methyl-butyl]-2-fluoro-acetamide;
(2R)-2-chloro-N-[(1S,2S)-1-[(1R,2S,5S)-2-[[[(2R)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2S)-2-chloro-N-[(1S,2S)-1-[(1R,2S,5S)-2-[[[(2S)-2-chloro-2-fluoro-acetyl]-[[(3S)-2-oxo-3-piperidyl]methyl]amino]carbamoyl]-6,6-dimethyl-3-azabicyclo[3.1.0]hexane-3-carbonyl]-2-methyl-butyl]-2-fluoro-acetamide;
(2R)—N-[(1S,2S)-1-[(3S,3aS,6aR)-3-[[[(2R)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide; and
(2S)—N-[(1S,2S)-1-[(3S,3aS,6aR)-3-[[[(2S)-2-chloro-2-fluoro-acetyl]-[[(3S)-2-oxo-3-piperidyl]methyl]amino]carbamoyl]-3,3a,4,5,6,6a-hexahydro-1H-cyclopenta[c]pyrrole-2-carbonyl]-2-methyl-butyl]-2-chloro-2-fluoro-acetamide.

50. A process of manufacturing a compound of formula (I) according to claim 32, or a pharmaceutically acceptable salt thereof, comprising:
(a) reacting a compound of formula (XI) 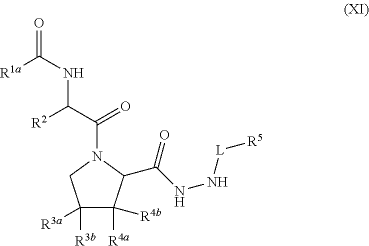
wherein R^{1a}, R², R^{3a}, R^{3b}, R^{4a}, R^{4b}, R⁵, and L are as defined in claim 1;
with a compound of formula (XI-1), 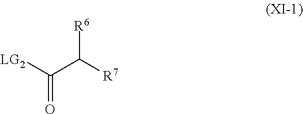
wherein R⁶ and R⁷ are as defined in claim 1 and LG₂ is a leaving group;
in the presence of a coupling reagent and a base;
to form a compound of formula (I-a) 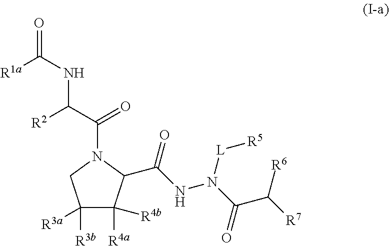
wherein R^{1a}, R², R^{3a}, R^{3b}, R^{4a}, R^{4b}, R⁵, R⁶, R⁷, and L are as defined in claim 1; or
(b) reacting a compound of formula (XXIII) 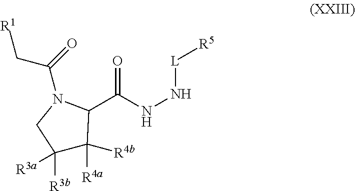
wherein R¹, R^{3a}, R^{3b}, R^{4a}, R^{4b}, R⁵, and L are as defined in claim 1;
with a compound of formula (XI-1), 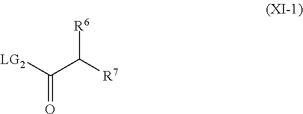
wherein R⁶ and R⁷ are as defined in claim 1 and LG₂ is a leaving group;
in the presence of a coupling reagent and a base;
to form a compound of formula (I-b) 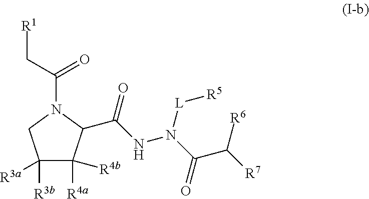
wherein R¹, R^{3a}, R^{3b}, R^{4a}, R^{4b}, R⁵, R⁶, R⁷, and L are as defined in claim 1.

51. A compound of formula (XI), or a salt thereof, 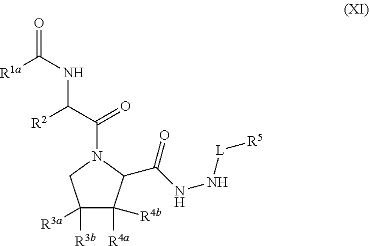
wherein R^{1a}, R², R^{3a}, R^{3b}, R^{4a}, R^{4b}, R⁵, and L are as defined in claim 1.

52. A compound of formula (XXIII), or a salt thereof, 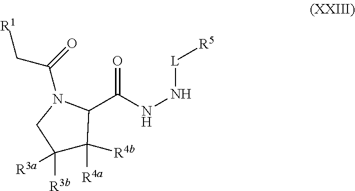
wherein R¹, R^{3a}, R^{3b}, R^{4a}, R^{4b}, R⁵, and L are as defined in claim 1.

53. A pharmaceutical composition comprising a compound of formula (I) according to claim 32, or a pharmaceutically acceptable salt thereof, and a therapeutically inert carrier.

54. A method of treatment or prophylaxis of coronavirus infections, said method comprising administering a therapeutically active amount of a compound of formula (I) according to claim 32 or a pharmaceutically acceptable salt thereof, to a subject in need thereof.

55. A method of inhibiting the enzymatic activity of 3C-like proteases, said method comprising contacting a 3C-like protease with a compound of formula (I) according to claim 32, or a pharmaceutically acceptable salt thereof.
